SEQUENCE LISTING (135 sequences)

SEQ ID NO: 1 - DNA, 822 bp, Homo sapiens
  misc_feature (327)..(327): unknown nucleotide
gaggcgccttgggaccgcgtgggagccgcagccgaaccgagtagggaccgggaccgcgcggcgccgccgtccccggccgggcccggcccccgcgagccgagcgcgcgcccccgtcgcccacccgggcgcggctggatgcggcggggtccccgcggcggcgacccccggccccgagcgcccggagcgcccagaggcggcgtgcggggcccggggacgccgcgccctstbgtgcgccgaggcgcgccccgagacagccgggggcccgcgccgcagccgccgcccgcgctgagccccggcccggcccgcggcccgcgcccggcggcagcntgagccaggccgagctgtccacctgctccgcgccgcagacgcagcgcatcttccaggaggctgtgcgcnagggcaacacgcaggagctgcagtygctgctgcagaacatgaccaactgcgagttcaacgtgaactcgttcgggcccgagggccagacggcgctgcaccagtcggtcatcgtcggcaacctggtgctcgtgaagctgctggtcaagttcggcgccgacatccgcctggccaaccgcgacggctggagcgcgctgcamatcgccgcgttcggtggccaccaggacatcgtgctctatctcatcaccaaggcgaagtacgcggccagcgcsggtgtatgcccgccgggaccccggaccccggccctgcgcccgcgtcgtctctgctgtaccttcccgccaactacctcggtgcgcgcmcggctcgcaggccccgccagaaggcccgtggcaacggcgaatacggcgcgtgcgtcmcggccccagggtc

SEQ ID NO: 2 - DNA, 9046 bp, Homo sapiens
ccttgtttgtgtgctaggctgggggggagagagggcgagagagagcgggcgagagtgggcaagcaggacgccgggctgagtgctaactgcgggacgcagagagtgcggaggggagtcgggtcggagagaggcggcaggggccagaacagtggcagggggcccggggcgcacgggctgaggcgacccccagccccctcccgtccgcacacacccccaccgcggtccagcagccgggccggcgtcgacgctaggggggaccattacataacccgcgccccggccgtcttctcccgccgccgcggcgcccgaactgagcccggggcgggcgctccagcactggccgccggcgtggggcgtagcagcggccgtattattatttcgcggaaaggaaggcgaaggaggggagcgccggcgcgaggaggggccgcctgcgcccgccgccggagcggggcctcctcggtgggctccgcgtcggcgcgggcgtgcgggcggcgctgctcggcccggccccctcggccctctggtccggccagctccgctcccggcgtccttgccgcgcctccgccggccgccgcgcgatgtgaggcggcggcgccagcctggctctcggctcgggcgagttctctgcggccattaggggccggtgcggcggcggcgcggagcgcggcggcaggaggagggttcggagggtgggggcgcaggcccgggagggggcaccgggaggaggtgagtgtctcttgtcgcctcctcctctccccccttttcgcccccgcctccttgtggcgatgagaaggaggaggacagcgccgaggaggaagaggttgatggcggcggcggagctccgagagacctcggctgggcaggggccggccgtggcgggccggggactgcgcctctagagccgcgagttctcgggaattcgccgcagcggaccggcctcggcgaatttgtgctcttgtgccctcctccgggcttgggccaggccggcccctcgcacttgcccttaccttttctatcgagtccgcatccctctccagccactgcgacccggcgaagagaaaaaggaacttcccccaccccctcgggtgccgtcggagccccccagcccacccctgggtgcggcgcggggaccccgggccgaagaagagatttcctgaggattctggttttcctcgcttgtatctccgaaagaattaaaaatggccgagaatgtggtggaaccggggccgccttcagccaagcggcctaaactctcatctccggccctctcggcgtccgccagcgatggcacagattttggctctctatttgacttggagcacgacttaccagatgaattaatcaactctacagaattgggactaaccaatggtggtgatattaatcagcttcagacaagtcttggcatggtacaagatgcagcttctaaacataaacagctgtcagaattgctgcgatctggtagttcccctaacctcaatatgggagttggtggcccaggtcaagtcatggccagccaggcccaacagagcagtcctggattaggtttgataaatagcatggtcaaaagcccaatgacacaggcaggcttgacttctcccaacatggggatgggcactagtggaccaaatcagggtcctacgcagtcaacaggtatgatgaacagtccagtaaatcagcctgccatgggaatgaacacagggacgaatgcgggcatgaatcctggaatgttggctgcaggcaatggacaagggataatgcctaatcaagtcatgaacggttcaattggagcaggccgagggcgacaggatatgcagtacccaaacccaggcatgggaagtgctggcaacttactgactgagcctcttcagcagggctctccccagatgggaggacaaacaggattgagaggcccccagcctcttaagatgggaatgatgaacaaccccaatccttatggttcaccatatactcagaatcctggacagcagattggagccagtggccttggtctccagattcagacaaaaactgtactatcaaataacttatctccatttgctatggacaaaaaggcagttcctggtggaggaatgcccaacatgggtcaacagccagccccgcaggtccagcagccaggtctggtgactccagttgcccaagggatgggttctggagcacatacagctgatccagagaagcgcaagctcatccagcagcagcttgttctccttttgcatgctcacaagtgccagcgccgggaacaggccaatggggaagtgaggcagtgcaaccttccccactgtcgcacaatgaagaatgtcctaaaccacatgacacactgccagtcaggcaagtcttgccaagtggcacactgtgcatcttctcgacaaatcatttcacactggaagaattgtacaagacatgattgtcctgtgtgtctccccctcaaaaatgctggtgataagagaaatcaacagccaattttgactggagcacccgttggacttggaaatcctagctctctaggggtgggtcaacagtctgcccccaacctaagcactgttagtcagattgatcccagctccatagaaagagcctatgcagctcttggactaccctatcaagtaaatcagatgccgacacaaccccaggtgcaagcaaagaaccagcagaatcagcagcctgggcagtctccccaaggcatgcggcccatgagcaacatgagtgctagtcctatgggagtaaatggaggtgtaggagttcaaacgccgagtcttctttctgactcaatgttgcattcagccataaattctcaaaacccaatgatgagtgaaaatgccagtgtgccctccctgggtcctatgccaacagcagctcaaccatccactactggaattcggaaacagtggcacgaagatattactcaggatcttcgaaatcatcttgttcacaaactcgtccaagccatatttcctacgccggatcctgctgctttaaaagacagacggatggaaaacctagttgcatatgctcggaaagttgaaggggacatgtatgaatctgcaaacaatcgagcggaatactaccaccttctagctgagaaaatctataagatccagaaagaactagaagaaaaacgaaggaccagactacagaagcagaacatgctaccaaatgctgcaggcatggttccagtttccatgaatccagggcctaacatgggacagccgcaaccaggaatgacttctaatggccctctacctgacccaagtatgatccgtggcagtgtgccaaaccagatgatgcctcgaataactccacaatctggtttgaatcaatttggccagatgagcatggcccagccccctattgtaccccggcaaacccctcctcttcagcaccatggacagttggctcaacctggagctctcaacccgcctatgggctatgggcctcgtatgcaacagccttccaaccagggccagttccttcctcagactcagttcccatcacagggaatgaatgtaacaaatatccctttggctccgtccagcggtcaagctccagtgtctcaagcacaaatgtctagttcttcctgcccggtgaactctcctataatgcctccagggtctcaggggagccacattcactgtccccagcttcctcaaccagctcttcatcagaattcaccctcgcctgtacctagtcgtacccccacccctcaccatactcccccaagcataggggctcagcagccaccagcaacaacaattccagcccctgttcctacaccaccagccatgccacctgggccacagtcccaggctctacatccccctccaaggcagacacctacaccaccaacaacacaacttccccaacaagtgcagccttcacttcctgctgcaccttctgctgaccagccccagcagcagcctcgctcacagcagagcacagcagcgtctgttcctaccccaaacgcaccgctgcttcctccgcagcctgcaactccactttcccagccagctgtaagcattgaaggacaggtatcaaatcctccatctactagtagcacagaagtgaattctcaggccattgctgagaagcagccttcccaggaagtgaagatggaggccaaaatggaagtggatcaaccagaaccagcagatacgcagccggaggatatttcagagtctaaagtggaagactgtaaaatggaatctaccgaaacagaagagagaagcactgagttaaaaactgaaataaaagaggaggaagaccagccaagtacttcagctacccagtcatctccggctccaggacagtcaaagaaaaagattttcaaaccagaagaactacgacaggcactgatgccaacattggaggcactttaccgtcaggatccagaatcccttccctttcgtcaacctgtggaccctcagcttttaggaatccctgattactttgatattgtgaagagccccatggatctttctaccattaagaggaagttagacactggacagtatcaggagccctggcagtatgtcgatgatatttggcttatgttcaataatgcctggttatataaccggaaaacatcacgggtatacaaatactgctccaagctctctgaggtctttgaacaagaaattgacccagtgatgcaaagccttggatactgttgtggcagaaagttggagttctctccacagacactgtgttgctacggcaaacagttgtgcacaatacctcgtgatgccacttattacagttaccagaacaggtatcatttctgtgagaagtgtttcaatgagatccaaggggagagcgtttctttgggggatgacccttcccagcctcaaactacaataaataaagaacaattttccaagagaaaaaatgacacactggatcctgaactgtttgttgaatgtacagagtgcggaagaaagatgcatcagatctgtgtccttcaccatgagatcatctggcctgctggattcgtctgtgatggctgtttaaagaaaagtgcacgaactaggaaagaaaataagttttctgctaaaaggttgccatctaccagacttggcacctttctagagaatcgtgtgaatgactttctgaggcgacagaatcaccctgagtcaggagaggtcactgttagagtagttcatgcttctgacaaaaccgtggaagtaaaaccaggcatgaaagcaaggtttgtggacagtggagagatggcagaatcctttccataccgaaccaaagccctctttgcctttgaagaaattgatggtgttgacctgtgcttctttggcatgcatgttcaagagtatggctctgactgccctccacccaaccagaggagagtatacatatcttacctcgatagtgttcatttcttccgtcctaaatgcttgaggactgcagtctatcatgaaatcctaattggatatttagaatatgtcaagaaattaggttacacaacagggcatatttgggcatgtccaccaagtgagggagatgattatatcttccattgccatcctcctgaccagaagatacccaagcccaagcgactgcaggaatggtacaaaaaaatgcttgacaaggctgtatcagagcgtattgtccatgactacaaggatatttttaaacaagctactgaagatagattaacaagtgcaaaggaattgccttatttcgagggtgatttctggcccaatgttctggaagaaagcattaaggaactggaacaggaggaagaagagagaaaacgagaggaaaacaccagcaatgaaagcacagatgtgaccaagggagacagcaaaaatgctaaaaagaagaataataagaaaaccagcaaaaataagagcagcctgagtaggggcaacaagaagaaacccgggatgcccaatgtatctaacgacctctcacagaaactatatgccaccatggagaagcataaagaggtcttctttgtgatccgcctcattgctggccctgctgccaactccctgcctcccattgttgatcctgatcctctcatcccctgcgatctgatggatggtcgggatgcgtttctcacgctggcaagggacaagcacctggagttctcttcactccgaagagcccagtggtccaccatgtgcatgctggtggagctgcacacgcagagccaggaccgctttgtctacacctgcaatgaatgcaagcaccatgtggagacacgctggcactgtactgtctgtgaggattatgacttgtgtatcacctgctataacactaaaaaccatgaccacaaaatggagaaactaggccttggcttagatgatgagagcaacaaccagcaggctgcagccacccagagcccaggcgattctcgccgcctgagtatccagcgctgcatccagtctctggtccatgcttgccagtgtcggaatgccaattgctcactgccatcctgccagaagatgaagcgggttgtgcagcataccaagggttgcaaacggaaaaccaatggcgggtgccccatctgcaagcagctcattgccctctgctgctaccatgccaagcactgccaggagaacaaatgcccggtgccgttctgcctaaacatcaagcagaagctccggcagcaacagctgcagcaccgactacagcaggcccaaatgcttcgcaggaggatggccagcatgcagcggactggtgtggttgggcagcaacagggcctcccttcccccactcctgccactccaacgacaccaactggccaacagccaaccaccccgcagacgccccagcccacttctcagcctcagcctacccctcccaatagcatgccaccctacttgcccaggactcaagctgctggccctgtgtcccagggtaaggcagcaggccaggtgacccctccaacccctcctcagactgctcagccaccccttccagggcccccacctacagcagtggaaatggcaatgcagattcagagagcagcggagacgcagcgccagatggcccacgtgcaaatttttcaaaggccaatccaacaccagatgcccccgatgactcccatggcccccatgggtatgaacccacctcccatgaccagaggtcccagtgggcatttggagccagggatgggaccgacagggatgcagcaacagccaccctggagccaaggaggattgcctcagccccagcaactacagtctgggatgccaaggccagccatgatgtcagtggcccagcatggtcaacctttgaacatggctccacaaccaggattgggccaggtaggtatcagcccactcaaaccaggcactgtgtctcaacaagccttacaaaaccttttgcggactctcaggtctcccagctctcccctgcagcagcaacaggtgcttagtatccttcacgccaacccccagctgttggctgcattcatcaagcagcgggctgccaagtatgccaactctaatccacaacccatccctgggcagcctggcatgccccaggggcagccagggctacagccacctaccatgccaggtcagcagggggtccactccaatccagccatgcagaacatgaatccaatgcaggcgggcgttcagagggctggcctgccccagcagcaaccacagcagcaactccagccacccatgggagggatgagcccccaggctcagcagatgaacatgaaccacaacaccatgccttcacaattccgagacatcttgagacgacagcaaatgatgcaacagcagcagcaacagggagcagggccaggaataggccctggaatggccaaccataaccagttccagcaaccccaaggagttggctacccaccacagccgcagcagcggatgcagcatcacatgcaacagatgcaacaaggaaatatgggacagataggccagcttccccaggccttgggagcagaggcaggtgccagtctacaggcctatcagcagcgactccttcagcaacagatggggtcccctgttcagcccaaccccatgagcccccagcagcatatgctcccaaatcaggcccagtccccacacctacaaggccagcagatccctaattctctctccaatcaagtgcgctctccccagcctgtcccttctccacggccacagtcccagcccccccactccagtccttccccaaggatgcagcctcagccttctccacaccacgtttccccacagacaagttccccacatcctggactggtagctgcccaggccaaccccatggaacaagggcattttgccagcccggaccagaattcaatgctttctcagcttgctagcaatccaggcatggcaaacctccatggtgcaagcgccacggacctgggactcagcaccgataactcagacttgaattcaaacctctcacagagtacactagacatacactagagacaccttgtattttgggagcaaaaaaattattttctcttaacaagactttttgtactgaaaacaatttttttgaatctttcgtagcctaaaagacaattttccttggaacacataagaactgtgcagtagccgtttgtggtttaaagcaaacatgcaagatgaacctgagggatgatagaatacaaagaatatatttttgttatgggctggttaccaccagcctttcttcccctttgtgtgtgtggttcaagtgtgcactgggaggaggctgaggcctgtgaagccaaacaatatgctcctgccttgcacctccaataggttttattattttttttaaattaatgaacatatgtaatattaatgaacatatgtaatattaatagttattatttactggtgcagatggttgacatttttccctattttcctcactttatggaagagttaaaacatttctaaaccagaggacaaaaggggttaatgttactttgaaattacattctatatatatataaatatatataaatatatattaaaataccagttttttttctctgggtgcaaagatgttcattcttttaaaaaatgtttaaaaaaaa

SEQ ID NO: 3 - DNA, 1918 bp, Homo sapiens
cggtgccgcggggatggcgggagccggagctggagccggagctcgcggcggagcggcggcgggggtcgaggctcgagctcgcgatccaccgcccgcgcaccgcgcacatcctcgccaccctcggcctgcggctcagccctcggcccgcaggatggatggcgggtcagggggcctggggtctggggacaacgccccgaccactgaggctcttttcgtggcactgggcgcgggcgtgacggcgctcagccatcccctgctctacgtgaagctgctcatccaggtgggtcatgagccgatgccccccacccttgggaccaatgtgctggggaggaaggtcctctatctgccgagcttcttcacctacgccaagtacatcgtgcaagtggatggtaagatagggctgttccgaggcctgagtccccggctgatgtccaacgccctctctactgtgactcggggtagcatgaagaaggttttccctccagatgagattgagcaggtttccaacaaggatgatatgaagacttccctgaagaaagttgtgaaggagacctcctacgagatgatgatgcagtgtgtgtcccgcatgttggcccaccccctgcatgtcatctcaatgcgctgcatggtccagtttgtgggacgggaggccaagtacagtggtgtgctgagctccattgggaagattttcaaagaggaagggctgctgggattcttcgttggattaatccctcacctcctgggcgatgtggttttcttgtggggctgtaacctgctggcccacttcatcaatgcctacctggtggatgacagcgtgagtgacaccccaggggggctgggaaacgaccagaatccaggttcccagttcagccaggccctggccatccggagctataccaagttcgtgatggggattgcagtgagcatgctgacctaccccttcctgctagttggcgacctcatggctgtgaacaactgcgggctgcaagctgggctccccccttactccccagtgttcaaatcctggattcactgctggaagtacctgagtgtgcagggccagctcttccgaggctccagcctgcttttccgccgggtgtcatcaggatcatgctttgccctggagtaacctgaatcatctaaaaaacacggtctcaacctggccaccgtgggtgaggcctgaccaccttgggacacctgcaagacgactccaacccaacaacaaccagatgtgctccagcccagccgggcttcagttccatatttgccatgtgtctgtccagatgtggggttgagcgggggtggggctgcacccagtggattgggtcacccggcagacctagggaaggtgaggcgaggtggggagttggcagaatccccatacctcgcagatttgctgagtctgtcttgtgcagagggccagagaatggcttatgggggcccaggttggatggggaaaggctaatggggtcagaccccaccccgtctacccctccagtcagcccagcgcccatcctgcagctcagctgggagcatcattctcctgctttgtacatagggtgtggtcccctggcacgtggccaccatcatgtctaggcctatgctaggaggcaaatggccagctctgcctgtgtttttctcaacactacttttctgatatgagggcagcacctgcctctgaatgggaaatcatgcaactactcagaatgtgtcctcctcatctaatgctcatctgtttaatggtgatgcctcgcgtacaggatctggttacctgtgcagttgtgaatacccagaggttgggcagatcagtgtctctagtcctacccagttttaaagttcatggtaagatttgacctcatctcccgcaaataaatgtattggtgatttggaaaaaaaaaaaaaaaaaaaaaaaaaaaaa

SEQ ID NO: 4 - DNA, 1254 bp, Homo sapiens
atgacagagttacctgcaccgttgtcctacttccagaatgcacagatgtctgaggacaaccacctgagcaatactgtacgtagccagaatgacaatagagaacggcaggagcacaacgacagacggagccttggccaccctgagccattatctaatggacgaccccagggtaactcccggcaggtggtggagcaagatgaggaagaagatgaggagctgacattgaaatatggcgccaagcatgtgatcatgctctttgtccctgtgactctctgcatggtggtggtcgtggctaccattaagtcagtcagcttttatacccggaaggatgggcagctaatctataccccattcacagaagataccgagactgtgggccagggagccctgcactcaattctgaatgctgccatcatgatcagtgtcattgttgtcatgactatcctcctggtggttctgtataaatacaggtgctataaggtcatccatgcctggcttattatatcatctctattgttgctgttctttttttcattcatttacttgggggaagtgtttaaaacctataacgttgctgtggactacattactgttgcactcctgatctggaattttggtgtggtgggaatgatttccattcactggaaaggtccacttcgactccagcaggcatatctcattatgattagtgccctcatggccctggtgtttatcaagtacctccctgaatggactgcgtggctcatcttggctgtgatttcggtatatgatttagtggctgttttgtgtccgaaaggtccacttcgtatgctggttgaaacagctcaggagagaaatgaaacgctttttccagctctcatttactcctcaacaatggtgtggttggtgaatatggcagaaggagacccggaagctcaaaggagagtatccaaaaattccaagtataatgcagaaagagcctgtctgcctcctgctgccatcaacctgctgtctatagctcccatggcacccaggctgttcatgccaaagggtgcctgcaggcccacggcacagaaagggagtcacaagacactgttgcagagaatgatgatggcgggttcagtgaggaatgggaagcccagagggacagtcatctagggcctcatcgctctacacctgagtcacgagctgctgtccaggaactttccagcagtatcctcgctggtgaagacccagaggaaaggggagtaaaacttggattgggagatttcattt

SEQ ID NO: 5 - DNA, 1380 bp, Homo sapiens
cgagcggcggcggagcaggcatttccagcagtgaggagacagccagaagcaagctattggagctgaaggaacctgagacagaagctagtcccccctctgaattttactgatgaagaaactgaggccacagagctaaagtgacttttcccaaggtcgcccagcgaggacgtgggacttctcagacgtcaggagagtgatgtgagggagctgtgtgaccatagaaagtgacgtgttaaaaaccagcgctgccctctttgaaagccagggagcatcattcatttagcctgctgagaagaagaaaccaagtgtccgggattcagacctctctgcggccccaagtgttcgtggtgcttccagaggcagggctatgctcacattcatggcctctgacagcgaggaagaagtgtgtgatgagcggacgtccctaatgtcggccgagagccccacgccgcgctcctgccaggagggcaggcagggcccagaggatggagagaacactgcccagtggagaagccaggagaacgaggaggacggtgaggaggaccctgaccgctatgtctgtagtggggttcccgggcggccgccaggcctggaggaagagctgaccctcaaatacggagcgaagcacgtgatcatgctgtttgtgcctgtcactctgtgcatgatcgtggtggtagccaccatcaagtctgtgcgcttctacacagagaagaatggacagctcatctacacgacattcactgaggacacaccctcggtgggccagcgcctcctcaactccgtgctgaacaccctcatcatgatcagcgtcatcgtggttatgaccatcttcttggtggtgctctacaagtaccgctgctacaagttcatccatggctggttgatcatgtcttcactgatgctgctgttcctcttcacctatatctaccttggggaagtgctcaagacctacaatgtggccatggactaccccaccctcttgctgactgtctggaacttcggggcagtgggcatggtgtgcatccactggaagggccctctggtgctgcagcaggcctacctcatcatgatcagtgcgctcatggccctagtgttcatcaagtacctcccagagtggtccgcgtgggtcatcctgggcgccatctctgtgtatgatctcgtggctgtgctgtgtcccaaagggcctctgagaatgctggtagaaactgcccaggagagaaatgagcccatattccctgccctgatatactcatctgccatggtgtggacggttggcatggcgaagctggacccctcctctcagggtgccctccagctcccctacgacccggagatggaagaagactcctatgacagttttggggagccttcataccccga

SEQ ID NO: 6 - DNA, 3571 bp, Homo sapiens
  misc_feature (2506)..(2506): unknown nucleotide
ctcagcaggtcaggacatttggtaggggaaggttgaaagacaaaagcagcaggccttgggttctcagccttttaaaaactattattaaatatatatttttaaaatttagtggttagagcttttagtaatgtgcctgtattacatgtagagagtattcgtcaaccaagaggagttttaaaatgtcaaaaccgggaaaacctactctaaaccatggcttggttcctgttgatcttaaaagtgcaaaagagcctctaccacatcaaaccgtgatgaagatatttagcattagcatcattgcccaaggcctccctttttgtcgaagacggatgaaaagaaagttggaccatggttctgaggtccgctctttttctttgggaaagaaaccatgcaaagtctcagaatatacaagtaccactgggcttgtaccatgttcagcaacaccaacaacttttggggacctcagagcagccaatggccaagggcaacaacgacgccgaattacatctgtccagccacctacaggcctccaggaatggctaaaaatgtttcagagctggagtggaccagagaaattgcttgctttagatgaactcattgatagttgtgaaccaacacaagtaaaacatatgatgcaagtgatagaaccccagtttcaacgagacttcatttcattgctccctaaagagttggcactctatgtgctttcattcctggaacccaaagacctgctacaagcagctcagacatgtcgctactggagaattttggctgaagacaaccttctctggagagagaaatgcaaagaagaggggattgatgaaccattgcacatcaagagaagaaaagtaataaaaccaggtttcatacacagtccatggaaaagtgcatacatcagacagcacagaattgatactaactggaggcgaggagaactcaaatctcctaaggtgctgaaaggacatgatgatcatgtgatcacatgcttacagttttgtggtaaccgaatagttagtggttctgatgacaacactttaaaagtttggtcagcagtcacaggcaaatgtctgagaacattagtgggacatacaggtggagtatggtcatcacaaatgagagacaacatcatcattagtggatctacagatcggacactcaaagtgtggaatgcagagactggagaatgtatacacaccttatatgggcatacttccactgtgcgttgtatgcatcttcatgaaaaaagagttgttagcggttctcgagatgccactcttagggtttgggatattgagacaggccagtgtttacatgttttgatgggtcatgttgcagcagtccgctgtgttcaatatgatggcaggagggttgttagtggagcatatgattttatggtaaaggtgtgggatccagagactgaaacctgtctacacacgttgcaggggcatactaatagagtctattcattacagtttgatggtatccatgtggtgagtggatctcttgatacatcaatccgtgtttgggatgtggagacagggaattgcattcacacgttaacagggcaccagtcgttaacaagtggaatggaactcaaagacaatattcttgtctctgggaatgcagattctacagttaaaatctgggatatcaaaacaggacagtgtttacaaacattgcaaggtcccaacaagcatcagagtgctgtgacctgtttacagttcaacaagaactttgtaattaccagctcagatgatggaactgtaaaactatgggacttgaaaacgggtgaatttattcgaaacctagtcacattggagagtggggggagtgggggagttgtgtggcggatcagagcctcaaacacaaagctggtgtgtgcagttgggagtcggaatgggactgaagaaaccaagctgctggtgctggactttgatgtggacatgaagtgaagagcagaaaagatgaatttgtccaattgtgtagacgatatactccctgcccttccccctgcaaaaagaaaaaaagaaaagaaaaagaaaaaaatcccttgttctcagtggtgcaggatgttggcttggggcaacagattgaaaagacctacagactaagaaggaaaagaagaagagatgacaaaccataactgacaagagaggcgtctgctgtctcatcacataaaaggcttcacttttgactgagggcagctttgcaaaatgagactttctaaatcaaaccaggtgcaattatttctttattttcttctccagtggtcattggggcagtgttaatgctgaaacatcattacagattctgctagcctgttcttttaccactgacagctagacacctagaaaggaactgcaataatatcaaaacaagtactggttgactttctaattagagagcatctgcaacaaaaagtcatttttctggagtggaaaagcttaaaaaaattactgtgaattgtttttgtacagttatcatgaaaagcttttttttttattttttngccaaccattgccaatgtcaatcaatcacagtattagcctctgttaatctatttactgttgcttccatatacattcttcaatgcatatgttgctcaaaggtggcaagttgtcctgggttctgtgagtcctgagatggatttaattcttgatgctggtgctagaagtaggtcttcaaatatgggattgttgtcccaaccctgtactgtactcccagtggccaaacttatttatgctgctaaatgaaagaaagaaaaaagcaaattattttttttattttttttctgctgtgacgttttagtcccagactgaattccaaatttgctctagtttggttatggaaaaaagactttttgccactgaaacttgagccatctgtgcctctaagaggctgagaatggaagagtttcagataataaagagtgaagtttgcctgcaagtaaagaattgagagtgtgtgcaaagcttattttcttttatctgggcaaaaattaaaacacattccttggaacagagctattacttgcctgttctgtggagaaacttttctttttgagggctgtggtgaatggatgaacgtacatcgtaaaactgacaaaatattttaaaaatatataaaacacaaaattaaaataaagttgctggtcagtcttagtgttttacagtatttgggaaaacaactgttacagttttattgctctgagtaactgacaaagcagaaactattcagtttttgtagtaaaggcgtcacatgcaaacaaacaaaatgaatgaaacagtcaaatggtttgcctcattctccaagagccacaactcaagctgaactgtgaaagtggtttaacactgtatcctaggcgatcttttttcctccttctgtttatttttttgnttgttttatttatagtctgatttaaaacaatcagattcaagttggttaattttagttatgtaacaacctgacatgatggaggaaaacaacctttaaagggattgtgtctatggtttgattcacttagaaattttattttcttataacttaagtgcaataaaatgtgttttttcatgtt

SEQ ID NO: 7 - DNA, 1257 bp, Homo sapiens
gtcgaccgcctgcccaggcccggggagggaggaggcgggcgtcagggtgctgcgccccgctcggcgtccgagcttccggccgggctgtgccccgcgcggtcttcgccgggatgaagcgcccctgcgaggagacgacctccgagagcgacatggacgagaccatcgacgtggggagcgagaacaattactcggggcaaagtactagctctgtgattagattgaattctccaacaacaacatctcagattatggcaagaaagaaaaggagagggattatagagaaaaggcgtcgggatcggataaataacagtttatctgagttgagaagacttgtgccaactgcttttgaaaaacaaggatctgcaaagttagaaaaagctgaaatattgcaaatgacagtggatcatttgaagatgcttcaggcaacagggggtaaaggctactttgacgcacacgctcttgccatggacttcatgagcataggattccgagagtgcctaacagaagttgcgcggtacctgagctccgtggaaggcctggactcctcggatccgctgcgggtgcggcttgtgtctcatctcagcacttgcgccacccagcgggaggcggcggccatgacatcctccatggcccaccaccatcatccgctccacccgcatcactgggccgccgccttccaccacctgcccgcagccctgctccagcccaacggcctccatgcctcagagtcaaccccttgtcgcctctccacaacttcagaagtgcctcctgcccacggctctgctctcctcacggccacgtttgcccatgcggattcagccctccgaatgccatccacgggcagcgtcgccccctgcgtgccacctctctccacctctctcttgtccctctctgccaccgtccacgccgcagccgcagcagccaccgcggctgcacacagcttccctctgtccttcgcgggggcattccccatgcttcccccaaacgcagcagcagcagtggccgcggccacagccatcagcccgcccttgtcagtatcagccacgtccagtcctcagcagaccagcagtggaacaaacaataaaccttaccgaccctgggggacagaagttggagctttttaaatttttcttgaacttcttgcaatagtaactgaatgtcctccatttcagagtcagcttaaaacctctgcaccctgaaggtagccatacagatgccgacagatccacaaaggaacaataaagctatttgagacac

SEQ ID NO: 8 - DNA, 2223 bp, Homo sapiens
tcagtgtgtgcggaacgcaagcagccgagagcggagaggcgccgctgtagttaactcctccctgcccgccgcgccgaccctccccaggaacccccagggagccagcatgaagcgagctcaccccgagtacagctcctcggacagcgagctggacgagaccatcgaggtggagaaggagagtgcggacgagaatggaaacttgagttcggctctaggttccatgtccccaactacatcttcccagattttggccagaaaaagacggagaggaataattgagaagcgccgacgagaccggatcaataacagtttgtctgagctgagaaggctggtacccagtgcttttgagaagcagggatctgctaagctagaaaaagccgagatcctgcagatgaccgtggatcacctgaaaatgctgcatacggcaggagggaaaggttactttgacgcgcacgcccttgctatggactatcggagtttgggatttcgggaatgcctggcagaagttgcgcgttatctgagcatcattgaaggactagatgcctctgacccgcttcgagttcgactggtttcgcatctcaacaactacgcttcccagcgggaagccgcgagcggcgcccacgcgggcctcggacacattccctgggggaccgtcttcggacatcacccgcacatcgcgcacccgctgttgctgccccagaacggccacgggaacgcgggcaccacggcctcacccacggaaccgcaccaccagggcaggctgggctcggcacatccggaggcgcctgctttgcgagcgccccctagcggcagcttcggaccggtgctccctgtggtcacctccgcctccaaactgtcgctgcctctgctctcctcagtggcctccctgtcggccttccccttctctttcggctccttccacttactgtctcccaatgcactgagcccttcagcacccacgcaggctgcaaaccttggcaagccctatagaccttgggggacggagatcggagctttttaaagaactgatgtagaatgagggaggggaaagtttaaaatcccagctgggctggactgttgccaacatcaccttaaagtcgtcagtaaaagtaaaaaggaaaaaggtacactttcagataattttttttttaaagactaaaggtttgttggtttacttttatcttttttaatgtttttttcatcatgtcatgtattagcagtttttaaaaactagttgttaaattttgttcaagacattaaattgaaatagtgagtataagccaacactttgtgataggtttgtactgtgcctaatttactttgtaaaccagaatgattccgtttttgcctcaaaatttggggaatcttaacatttaggtatttttggtctgtttttctccttgtatagttatggtctgtttttagaattaattttccaaaccactatgcttaatgttaacatgattctgtttgttaatattttgacagattaaggtgttgtataaataatattcttttggggggaggggaactatattgaattttatatttctgagcaaagcgttgacaaatcagatgatcagctttatccaagaaagaagactagtaaattgtctgcctcctatagcagaaaggtgaatgtacaaactgttggtggcctgaatccatctgaccagctgctggtatctgccaggactggcagttctgatttagttaggaggaccgctgataggttaggtctcatttggagtgttggtggaaaggaaactgaaggtaattgaatagaatacgcctgcatttaccagccccagcaacacaaagaatttttaatcacacggatctcaaattcacaaatgttaacatggataagtgatcatggtgtgcgagtggtcaattgagtagtacagtggaaactgttaaatgcataacctaattttcctgggactgccatattttcttttaactggaaatttttatgtgagttttccttttggtgcatggaactgtggttgccaaggtatttaaaagggctttcctgcctccttctctttgatttatttaatttgatttgggctataaaatatcatttttcaggtttattcttttagcaggtgtagttaaacgacctccactgaactgggtttgacctctgttgtactgatgtgttgtgactaaataaaaaagaaagaacaaagtaaaaaaaaaaaaaaaaaaaaaaaaaaa

SEQ ID NO: 9 - DNA, 1471 bp, Homo sapiens
atcacacaggatccggagctggtgctgataacagcggaatcccccgtctacctctctccttggtcctggaacagcgctactgatcaccaagtagccacaaaatataataaaccctcagcacttgctcagtagttttgtgaaagtctcaagtaaaagagacacaaacaaaaaattctttttcgtgaagaactccaaaaataaaattctctagagataaaaaaaaaaaaaaaaggaaaatgccagctgatataatggagaaaaattcctcgtccccggtggctgctaccccagccagtgtcaacacgacaccggataaaccaaagacagcatctgagcacagaaagtcatcaaagcctattatggagaaaagacgaagagcaagaataaatgaaagtctgagccagctgaaaacactgattttggatgctctgaagaaagatagctcgcggcattccaagctggagaaggcggacattctggaaatgacagtgaagcacctccggaacctgcagcgggcgcagatgacggctgcgctgagcacagacccaagtgtgctggggaagtaccgagccggcttcagcgagtgcatgaacgaggtgacccgcttcctgtccacgtgcgagggcgttaataccgaggtgcgcactcggctgctcggccacctggccaactgcatgacccagatcaatgccatgacctaccccgggcagccgcaccccgccttgcaggcgccgccaccgcccccaccgggacccggcggcccccagcacgcgccgttcgcgccgccgccgccactcgtgcccatccccgggggcgcggcgccccctcccggcggcgccccctgcaagctgggcagccaggctggagaggcggctaaggtgtttggaggcttccaggtggtaccggctcccgatggccagtttgctttcctcattcccaacggggccttcgcgcacagcggccctgtcatccccgtctacaccagcaacagcggcacctccgtgggccccaacgcagtgtcaccttccagcggcccctcgcttacggcggactccatgtggaggccgtggcggaactgagggggctcaggccacccctcctcctaaactccccaacccacctctcttccctccggactctaaacaggaacttgaatactgggagagaagaggacttttttgattaagtggttactttgtgtttttttaatttctaagaagttactttttgtagagagagctgtattaagtgactgaccatgcactatatttgtatatattttatatgttcatattggattgcgcctttgtattataaaagctcagatgacatttcgttttttacacgagatttcttttttatgtgatgccaaagatgtttgaaaatgctcttaaaatatcttcctttggggaagtttatttgagaaaatataataaaagaaaaaagtaaaggcaaaaaaaaaaaaaaaaaa

SEQ ID NO: 10 - DNA, 642 bp, Homo sapiens
  misc_feature (400)..(400): unknown nucleotide
gcgcggggacactcgtgcgactggggcaaggtgcccacgggcttctctcctaatttgcctcacgtaacagttgagaccccagagggcagcaaaactgggttcgaattggagagccgtccaggcacagacaaattcattcatctgcccagtgcccgctaactgcggttccaggcgccggcgacgcaattccagagctgccaccgcttccccgcggagcatggggctgcctcgccgggcaggggacgcggcggagctgcgcaagagcctgaagccgctgctggagaagcgccggcgcgcgcgcatcaaccagagcctgagccagcttaaggggctcatcctgccgctgctgggccgggaggatgcttctggctggcacacctggcttcccctccatgctcangaactgcttcctactctacatccaggctcctgagcagcccccagcttaagcttcagcagctccagggtaaatccatctccgcccagagctgactccggatctgccccagtgacaataacaagataaatgccttccattctcttcgtacttacaggatgtaatccacatatttacacagtgtttcatttgcggtaatgtttgtatagtttcttgttccttcgtacctatctgggaagcctgtg

SEQ ID NO: 11 - DNA, 666 bp, Homo sapiens
atggccgcagacacgccggggaaaccgagcgcctcgccgatggcaggagcgccggccagcgccagccggaccccagacaagccccggagcgcggccgagcaccgcaagtcctccaagccggtcatggagaagcggcgccgagcgcgtattaacgagagcctcgctcagctcaaaaccctcatcctggacgccctcagaaaagagagctcccgccactcgaagctggagaaggcggacatcctggagatgaccgtgagacacctgcggagcctgcgtcgcgtgcaggtgacggccgcgctcagcgccgacccggccgttctgggcaagtaccgcgccggcttccacgagtgtctggcggaggtgaaccgcttcctggccggctgcgagggcgtcccggccgacgtgcgctcccgcctgctgggccacctggcagcctgcctgcgccagctgggaccctcccgccgcccggcctcgctgtccccggctgcccccgcagaggccccagcgcccgaggtctacgcgggccgcccgctgctgccatcgctcggcggccccttccctctgctcgcgccgccgctgctgccgggtctgacccgggcgctgcccgccgcccccagggcggggccgcagggcccgggtgggccctggaggccgtggctgcgctga

SEQ ID NO: 12 - DNA, 2103 bp, Homo sapiens
cccaggaacccccagggagccagcatgaagcgagctcaccccgagtacagctcctcggacagcgagctggacgagaccatcgaggtggagaaggagagtgcggacgagaatggaaacttgagttcggctctaggttccatgtccccaactacatcttcccagattttggccagaaaaagacggagaggaataattgagaagcgccgacgagaccggatcaataacagtttgtctgagctgagaaggctggtacccagtgcttttgagaagcagggatctgctaagctagaaaaagccgagatcctgcagatgaccgtggatcacctgaaaatgctgcatacggcaggagggaaaggttactttgacgcgcacgcccttgctatggactatcggagtttgggatttcgggaatgcctggcagaagttgcgcgttatctgagcatcattgaaggactagatgcctctgacccgcttcgagttcgactggtttcgcatctcaacaactacgcttcccagcgggaagccgcgagcggcgcccacgcgggcctcggacacattccctgggggaccgtcttcggacatcacccgcacatcgcgcacccgctgttgctgccccagaacggccacgggaacgcgggcaccacggcctcacccacggaaccgcaccaccagggcaggctgggctcggcacatccggaggcgcctgctttgcgagcgccccctagcggcagcctcggaccggtgctccctgtggtcacctccgcctccaaactgtcgccgcctctgctctcctcagtggcctccctgtcggccttccccttctctttcggctccttccacttactgtctcccaatgcactgagcccttcagcacccacgcaggctgcaaaccttggcaagccctatagaccttgggggacggagatcggagctttttaaagaactgatgtagaatgagggaggggaaagtttaaaatcccagctgggctggactgttgccaacatcaccttaaagtcgtcagtaaaagtaaaaaggaaaaaggtacactttcagataattttttttttaaagactaaaggtttgttggtttacttttatcttttttaatgtttttttcatcatgtcatgtattagcagtttttaaaaactagttgttaaattttgttcaagacattaaattgaaatagtgagtataagccaacactttgtgataggtttgtactgtgcctaatttactttgtaaaccagaatgattccgtttttgcctcaaaatttggggaatcttaacatttagtatttttggtctgtttttctccttgtatagttatggtctgtttttagaattaattttccaaaccactatgcttaatgttaacatgattctgtttgttaatattttgacagattaaggtgttgtataaataatattcttttggggggaggggaactatattgaattttatatttctgagcaaagcgttgacaaatcagatgatcagctttatccaagaaagaagactagtaaattgtctgcctcctatagcagaaaggtgaatgtacaaactgttggtggccctgaatccatctgaccagctgctggtatctgccaggactggcagttctgatttagttaggagagagccgctgataggttaggtctcatttggagtgttggtggaaaggaaactgaaggtaattgaatagaatacgcctgcatttaccagccccagcaacacaaagaatttttaatcacacggatctcaaattcacaaatgttaacatggataagtgatcatggtgtgcgagtggtcaattgagtagtacagtggaaactgttaaatgcataacctaattttcctgggactgccatattttcttttaactggaaatttttatgtgagttttccttttggtgcatggaactgtggttgccaaggtatttaaaagggctttcctgcctccttctctttgatttatttaatttgatttgggctataaaatatcatttttcaggtttattcttttagcaggtgtagttaaacgacctccactgaactgggtttgacctctgttgtactgatgtgttgtgactaaataaaaaa

SEQ ID NO: 13 - DNA, 2223 bp, Homo sapiens; identical to SEQ ID NO: 8

SEQ ID NO: 14 - DNA, 2533 bp, Homo sapiens
tcggcgtccgagcttccggccgggctgtgccccgcgcggtcttcgccgggatgaagcgcccctgcgaggagacgacctccgagagcgacatggacgagaccatcgacgtggggagcgagaacaattactcggggcaaagtactagctctgtgattagattgaattctccaacaacaacatctcagattatggcaagaaagaaaaggagagggattatagagaaaaggcgtcgggatcggataaataacagtttatctgagttgagaagacttgtgccaactgcttttgaaaaacaaggatctgcaaagttagaaaaagctgaaatattgcaaatgacagtggatcatttgaagatgcttcaggcaacagggggtaaaggctactttgacgcacacgctcttgccatggacttcatgagcataggattccgagagtgcctaacagaagttgcgcggtacctgagctccgtggaaggcctggactcctcggatccgctgcgggtgcggcttgtgtctcatctcagcacttgcgccacccagcgggaggcggcggccatgacatcctccatggcccaccaccatcatccgctccacccgcatcactgggccgccgccttccaccacctgcccgcagccctgctccagcccaacggcctccatgcctcagagtcaaccccttgtcgcctctccacaacttcagaagtgcctcctgcccacggctctgctctcctcacggccacgtttgcccatgcggattcagccctccgaatgccatccacgggcagcgtcgccccctgcgtgccacctctctccacctctctcttgtccctctctgccaccgtccacgccgcagccgcagcagccaccgcggctgcacacagcttccctctgtccttcgcgggggcattccccatgcttcccccaaacgcagcagcagcagtggccgcggccacagccatcagcccgcccttgtcagtatcagccacgtccagtcctcagcagaccagcagtggaacaaacaataaaccttaccgaccctgggggacagaagttggagctttttaaatttttcttgaacttcttgcaatagtaactgaatgtcctccatttcagagtcagcttaaaacctctgcaccctgaaggtagccatacagatgccgacagatccacaaaggaacaataaagctatttgagacacaaacctcacgagtggaaatgtggtattctcttttttttctctcccttttttgtttggttcaaggcagctcggtaactgacatcagcaacttttgaaaacttcacacttgttaccatttagaagtttcctggaaaatatatggaccgtaccatccagcagtgcatcagtatgtctgaattggggaagtaaaatgccctgactgaattctcttgagactagatgggacatacatatatagagagagagtgagagagtcgtgtttcgtaagtgcctgagcttaggaagttttcttctggatatataacattgcacaagggaagacgagtgtggaggataggttaagaaaggaaagggacagaagtcttgcaataggctgcagacattttaataccatgccagagaagagtattctgctgaaaccaacaggttttactggtcaaaatgactgctgaaaataattttcaagttgaaagatctagttttatcttagtttgccttctttgtacagacatgccaagaggtgacatttagcagtgcattggtataagcaattatttcatcagttctcagattaacaagcatttctgctctgcctgcaggcccccaggcacttttttttttggatggctcaaaatatggtgctgctttatataaaccttacatttatatagtgcacctatgagcagttgcctaccatgtgtccaccagaggctatttaattcatgccaacttgaaaactctccagtttgtaggagtttggtttaatttattcagtttcattaggactatttttatatatttatcctcttcattttctcctaatgatgcaacatctattcttgtcaccctttgggagaagttacatttctggaggtgatgaagcaaggagggagcactaggaagagaaaagctacaatttttaaagctctttgtcaagttagtgattgcatttgatcccaaaacaagatgaatgtatgcaatgggatgtacataagttatttttgcccatgcctaaactagtgctatgtaatggggttgtggttttgtttttttcgatttcgtttaatgacaaaataatctcttaatatgctgaaatcaagcacgtgagagtttttgtttaaaagataagagacacagcatgtattatgcacttcatttctctactgtgtggagaaagcaataaacattatgagaatgttaaacgttatgcaaaattatacttttaaatatttgttttgaaattactgtacctagtcttttttgcattactttgtaacctttttctatgcaagagtctttacataccactaattaaatgaagtcctttttgactatt

SEQ ID NO: 15 - DNA, 3416 bp, Homo sapiens
accaggcagcctgcgttcgccatgaagcgacccaaggagccgagcggctccgacggggagtccgacggacccatcgacgtgggccaagagggccagctgagccagatggccaggccgctgtccacccccagctcttcgcagatgcaagccaggaagaaacgcagagggatcatagagaaacggcgtcgagaccgcatcaacagtagcctttctgaattgcgacgcttggtccccactgcctttgagaaacagggctcttccaagctggagaaagccgaggtcttgcagatgacggtggatcacttgaaaatgctccatgccactggtgggacaggattctttgatgcccgagccctggcagttgacttccggagcattggttttcgggagtgcctcactgaggtcatcaggtacctgggggtccttgaagggcccagcagccgtgcagaccccgtccggattcgccttctctcccacctcaacagctacgcagccgagatggagccttcgcccacgcccactggccctttggccttccctgcctggccctggtctttcttccatagctgtccagggctgccagccctgagcaaccagctcgccatcctgggaagagtgcccagccctgtcctccccggtgtctcctctcctgcttaccccatcccagccctccgaaccgctccccttcgcagagccacaggcatcatcctgccagcccggaggaatgtgctgcccagtcgaggggcatcttccacccggagggcccgccccctagagaggccagcgacccctgtgcctgtcgcccccagcagcagggctgccaggagcagccacatcgctcccctcctgcagtcttcctccccaacaccccctggtcctacagggtcggctgcttacgtggctgttcccacccccaactcatcctccccagggccagctgggaggccagcgggagccatgctctaccactcctgggtctctgaaatcactgaaatcggggctttctgagctgccccttcaccaccccgccccaaggaataaggaaggttcttttaccaggagcccaaaaaagggcactgccttttctgctttgcttcgtggactggctcatatgtgaaggcacgttctccagccatcagaggccccctcctcctccaacccatctctccttctcactgttatcccagcttatccacccagctctcctggagctgttctggtctcagaggcttggttccatttctcacctgaacagatgagtcctgggagagaccctcagagatccgcccagacccctctcctgccctctgcacaccagcagcaggcatgaaccttgggtctgggaaaaagctttaacctgcagggcaccaggacccaaggcaggctgttccttggggcggtcagaccccagtcaggagcaatgactgactggctgcagccttcccacgccaagaggctggaacatagtgtctgcctcgcttcctggagatagtaactgagcaggggctacaaagaggtctcctgggaaccctgtctgccccttcccacctgtccttgggccacaccatcacactgaaccacaggacagaccctttctccaccacagccaaggcctggagactgggggcccagcagagcctgctcccaccctcctcccagcagcagacacccaccctctcactgactaacaggtccctgcacacagctggcctggtaaacccagctgggaggtttctaggcagcagcaaaactctgtgacagggtgtcctcacaccaggccttggacagctctcccagacaggagccagggttgagcaatggagagcccagcccccacgtcttacagtcgccatcctccaggcgtgtggtccctccccattgggtgcacagtgcagaggggccgtggccccatgtgatggtgcgcagagaggaacctcttgggattcagcaccagacgtctgtgctgcctggtttgcatccggctcacagagcccagactgctggaacagccaaggactgtcaggctggacaaaaataactgcaaggaggggcaagagaaaggatgattcgaggcaccttggcccttcaaggtcatgcagtgggtcgagcgcctgagatcctgttcaccaggactccacagagctggctctgctcagaagccatttcattccccggctccaccctaggccactttttctaacagaggaaacaaatggtccagcagtcgttcccagcagaacagcggagcctggactgacacccagtgggaccagtgttgccacaccagttgataaaatgcagaaacccttctgtactcgttggtaaatatctactcccccaagtgactccaggtgccccccaccgcctggcacttcccccaggactcctacgatctggttactgcctggccgatccaaggctgtggagtcccagagccagcagttcactggtgctcattccacactggttagatacttcagttgtcacccctgggaagattctcccacctcctccctttgatggaaccaccctccccagaggctgcattgaggagactccacagactgaaaagtgagtttgcagaaaccttggggaaaagggccctttcaaagaagtggataagagggaggagatcattgagtgacccagaaagctcttttgaaaagacagactcctcaaggagagataaagaggaaagcacctctttcattttttagtgtgagctaattccatcagactgctgtcctcctggacccatctgagatgtgcagtagcaaggagaggggggatcattttagagagtgggtcattggcagggagtgctccggagggaggcagaggggagactgtggtagaaggaagacagaactcacacatgctcccaggattggggacagggacagaggaggtaacagaaggcaaaggccagtttccccgttatcatgaaggggcccactcaggacaggaacaaggacaactcctcctcctcctcctcctctcctgctgctcctgggataccaggtcagtgatgtagtcttgcagtttggcaacttcctagcctgagaatccctagtggggctgtgggaaacacatttccacgttgcaagcatgcaactccaaagaatctgtgatgccactgaaatgagatgggaatgatccagctctttcagcatcttggttgaacttgctttcattgtccctgggatattgtggaaggaaaggtgactgtgtgatctgattctgtggtcaaggacttgcatcttgtgtttctatccccaagccttcctggtgtctccaactcctaccccattgcatgggttgttgcggacatccaataaagatttttttagtgcttctggaaaaaaaaaaaaaaaaaaaaaaaaaaaaaaaaaaaaaaaaaaaaaaaaaaaaaaacc

SEQ ID NO: 16 - DNA, 2533 bp, Homo sapiens; identical to SEQ ID NO: 14

SEQ ID NO: 17 - DNA, 3416 bp, Homo sapiens; identical to SEQ ID NO: 15

SEQ ID NO: 18 - DNA, 915 bp, Homo sapiens
atgaagcgagctcaccccgagtacagctcctcggacagcgagctggacgagaccatcgaggtggagaaggagagtgcggacgagaatggaaacttgagttcggctctaggttccatgtccccaactacatcttcccagattttggccagaaaaagacggagaggaataattgagaagcgccgacgagaccggatcaataacagtttgtctgagctgagaaggctggtacccagtgcttttgagaagcagggatctgctaagctagaaaaagccgagatcctgcagatgaccgtggatcacctgaaaatgctgcatacggcaggagggaaaggttactttgacgcgcacgcccttgctatggactatcggagtttgggatttcgggaatgcctggcagaagttgcgcgttatctgagcatcattgaaggactagatgcctctgacccgcttcgagttcgactggtttcgcatctcaacaactacgcttcccagcgggaagccgcgagcggcgcccacgcgggcctcggacacattccctgggggaccgtcttcggacatcacccgcacatcgcgcacccgctgttgctgccccagaacggccacgggaacgcgggcaccacggcctcacccacggaaccgcaccaccagggcaggctgggctcggcacatccggaggcgcctgctttgcgagcgccccctagcggcagcctcggaccggtgctccctgtggtcacctccgcctccaaactgtcgccgcctctgctctcctcagtggcctccctgtcggccttccccttctctttcggctccttccacttactgtctcccaatgcactgagcccttcagcacccacgcaggctgcaaaccttggcaagccctatagaccttgggggacggagatcggagctttttaa

SEQ ID NO: 19 - DNA, 1014 bp, Homo sapiens
atgaagcgcccctgcgaggagacgacctccgagagcgacatggacgagaccatcgacgtggggagcgagaacaattactcggggcaaagtactagctctgtgattagattgaattctccaacaacaacatctcagattatggcaagaaagaaaaggagagggattatagagaaaaggcgtcgggatcggataaataacagtttatctgagttgagaagacttgtgccaactgcttttgaaaaacaaggatctgcaaagttagaaaaagctgaaatattgcaaatgacagtggatcatttgaagatgcttcaggcaacagggggtaaaggctactttgacgcacacgctcttgccatggacttcatgagcataggattccgagagtgcctaacagaagttgcgcggtacctgagctccgtggaaggcctggactcctcggatccgctgcgggtgcggcttgtgtctcatctcagcacttgcgccacccagcgggaggcggcggccatgacatcctccatggcccaccaccatcatccgctccacccgcatcactgggccgccgccttccaccacctgcccgcagccctgctccagcccaacggcctccatgcctcagagtcaaccccttgtcgcctctccacaacttcagaagtgcctcctgcccacggctctgctctcctcacggccacgtttgcccatgcggattcagccctccgaatgccatccacgggcagcgtcgccccctgcgtgccacctctctccacctctctcttgtccctctctgccaccgtccacgccgcagccgcagcagccaccgcggctgcacacagcttccctctgtccttcgcgggggcattccccatgcttcccccaaacgcagcagcagcagtggccgcggccacagccatcagcccgcccttgtcagtatcagccacgtccagtcctcagcagaccagcagtggaacaaacaataaaccttaccgaccctgggggacagaagttggagctttttaa

SEQ ID NO: 20 - DNA, 987 bp, Homo sapiens
atgaagcgacccaaggagccgagcggctccgacggggagtccgacggacccatcgacgtgggccaagagggccagctgagccagatggccaggccgctgtccacccccagctcttcgcagatgcaagccaggaagaaacgcagagggatcatagagaaacggcgtcgagaccgcatcaacagtagcctttctgaattgcgacgcttggtccccactgcctttgagaaacagggctcttccaagctggagaaagccgaggtcttgcagatgacggtggatcacttgaaaatgctccatgccactggtgggacaggattctttgatgcccgagccctggcagttgacttccggagcattggttttcgggagtgcctcactgaggtcatcaggtacctgggggtccttgaagggcccagcagccgtgcagaccccgtccggattcgccttctctcccacctcaacagctacgcagccgagatggagccttcgcccacgcccactggccctttggccttccctgcctggccctggtctttcttccatagctgtccagggctgccagccctgagcaaccagctcgccatcctgggaagagtgcccagccctgtcctccccggtgtctcctctcctgcttaccccatcccagccctccgaaccgctccccttcgcagagccacaggcatcatcctgccagcccggaggaatgtgctgcccagtcgaggggcatcttccacccggagggcccgccccctagagaggccagcgacccctgtgcctgtcgcccccagcagcagggctgccaggagcagccacatcgctcccctcctgcagtcttcctccccaacaccccctggtcctacagggtcggctgcttacgtggctgttcccacccccaactcatcctccccagggccagctgggaggccagcgggagccatgctctaccactcctgggtctctgaaatcactgaaatcggggctttctga

SEQ ID NO: 21 - DNA, 1331 bp, Homo sapiens
cggccgcgtcgacgggaaagagccgctagagcagaccgcgccgccgccggagccgcgcctgcccaggcccggggagggaggaggcgggcgtcagggtgctgcgccccgctcggcgtccgagcttccggccgggctgtgccccgcgcggtcttcgccgggatgaagcgcccctgcgaggagacgacctccgagagcgacatggacgagaccatcgacgtggggagcgagaacaattactcggggcaaagtactagctctgtgattagattgaattctccaacaacaacatctcagattatggcaagaaagaaaaggagagggattatagagaaaaggcgtcgggatcggataaataacagtttatctgagttgagaagacttgtgccaactgcttttgaaaaacaaggatctgcaaagttagaaaaagctgaaatattgcaaatgacagtggatcatttgaagatgcttcaggcaacagggggtaaaggctactttgacgcacacgctcttgccatggacttcatgagcataggattccgagagtgcctaacagaagttgcgcggtacctgagctccgtggaaggcctggactcctcggatccgctgcgggtgcggcttgtgtctcatctcagcacttgcgccacccagcgggaggcggcggccatgacatcctccatggcccaccaccatcatccgctccacccgcatcactgggccgccgccttccaccacctgcccgcagccctgctccagcccaacggcctccatgcctcagagtcaaccccttgtcgcctctccacaacttcagaagtgcctcctgcccacggctctgctctcctcacggccacgtttgcccatgcggattcagccctccgaatgccatccacgggcagcgtcgccccctgcgtgccacctctctccacctctctcttgtccctctctgccaccgtccacgccgcagccgcagcagccaccgcggctgcacacagcttccctctgtccttcgcgggggcattccccatgcttcccccaaacgcagcagcagcagtggccgcggccacagccatcagcccgcccttgtcagtatcagccacgtccagtcctcagcagaccagcagtggaacaaacaataaaccttaccgaccctgggggacagaagttggagctttttaaatttttcttgaacttcttgcaatagtaactgaatgtcctccatttcagagtcagcttaaaacctctgcaccctgaaggtagccatacagatgccgacagatccacaaaggaacaataaagctatttgagacacaaaaagtcgacgcggccgcgaattc

SEQ ID NO: 22 - DNA, 2234 bp, Homo sapiens
gaattcgcggccgcgtcgaccgagagcggagaggcgccgctgtagttaactcctccctgcccgccgcgccgaccctccccaggaacccccagggagccagcatgaagcgagctcaccccgagtacagctcctcggacagcgagctggacgagaccatcgaggtggagaaggagagtgcggacgagaatggaaacttgagttcggctctaggttccatgtccccaactacatcttcccagattttggccagaaaaagacggagaggaataattgagaagcgccgacgagaccggatcaataacagtttgtctgagctgagaaggctggtacccagtgcttttgagaagcagggatctgctaagctagaaaaagccgagatcctgcagatgaccgtggatcacctgaaaatgctgcatacggcaggagggaaaggttactttgacgcgcacgcccttgctatggactatcggagtttgggatttcgggaatgcctggcagaagttgcgcgttatctgagcatcattgaaggactagatgcctctgacccgcttcgagttcgactggtttcgcatctcaacaactacgcttcccagcgggaagccgcgagcggcgcccacgcgggcctcggacacattccctgggggaccgtcttcggacatcacccgcacatcgcgcacccgctgttgctgccccagaacggccacgggaacgcgggcaccacggcctcacccacggaaccgcaccaccagggcaggctgggctcggcacatccggaggcgcctgctttgcgagcgccccctagcggcagcctcggaccggtgctccctgtggtcacctccgcctccaaactgtcgccgcctctgctctcctcagtggcctccctgtcggccttccccttctctttcggctccttccacttactgtctcccaatgcactgagcccttcagcacccacgcaggctgcaaaccttggcaagccctatagaccttgggggacggagatcggagctttttaaagaactgatgtagaatgagggaggggaaagtttaaaatccagctgggctggactgttgccaacatcaccttaaagtcgtcagtaaaagtaaaaaggaaaaaggtacactttcagataattttttttttaaagactaaaggtttgttggtttacttttttcttttttaatgtttttttcatcatgtcatgtattagcagtttttaaaaaactagttgttaaattttgttcaagacattaaattgaaatagtgagtataagccaacactttgtgataggtttgtactgtgcctaatttactttgtaaaccagaatgattccgtttttgcctcaaaatttggggaatcttaacatttagtatttttggtctgtttttctccttgtatagttatggtctgtttttagaattaattttccaaaccactatgcttaatgttaacatgattctgtttgttaatattttgacagattaaggtgttgtataaataatattcttttggggggaggggaactatattgaattttatatttctgagcaaagcgttgacaaatcagatgatcagctttatccaagaaagaagactagtaaattgtctgcctcctatagcagaaaggtgaatgtacaaactgttggtggccctgaatccatctgaccagctgctggtatctgccaggactggcagttctgatttagttaggagagagccgctgataggttaggtctcatttggagtgttggtggaaaggaaactgaaggtaattgaatagaatacgcctgcatttaccagccccagcaacacaaagaatttttaatcacacggatctcaaattcacaaatgttaacatggataagtgatcatggtgtgcgagtggtcaattgagtagtacagtggaacctgttaaatgcataacctaattttcctgggactgccatattttcttttaactggaaatttttatgtgagttttccttttggtgcatggaactgtggttgccaaggtatttaaaagggctttcctgcctccttctctttgatttatttaatttgatttgggctataaaatatcatttttcaggtttattcttttagcaggtgtagttaaacgacctccactgaactgggtttgacctctgttgtactgatgtgttgtgactaaataaaaaagaaagaacaaagaaaaaaaaaaaaaaaaaaaaaaaaaaaaaaaaaaaaaaaaaa

SEQ ID NO: 23 - DNA, 1596 bp, Homo sapiens
agggtgctgcgccccgctcggcgtccgagcttccggccgggctgtgccccgcgcggtcttcgccgggatgaagcgcccctgcgaggagacgacctccgagagcgacatggacgagaccatcgacgtggggagcgagaacaattactcggggcaaagtactagctctgtgattagattgaattctccaacaacaacatctcagattatggcaagaaagaaaaggagagggattatagagaaaaggcgtcgggatcggataaataacagtttatctgagttgagaagacttgtgccaactgcttttgaaaaacaaggatctgcaaagttagaaaaagctgaaatattgcaaatgacagtggatcatttgaagatgcttcaggcaacagggggtaaaggctactttgacgcacacgctcttgccatggacttcatgagcataggattccgagagtgcctaacagaagttgcgcggtacctgagctccgtggaaggcctggactcctcggatccgctgcgggtgcggcttgtgtctcatctcagcacttgcgccacccagcgggaggcggcggccatgacatcctccatggcccaccaccatcatccgctccacccgcatcactgggccgccgccttccaccacctgcccgcagccctgctccagcccaacggcctccatgcctcagagtcaaccccttgtcgcctctccacaacttcagaagtgcctcctgcccacggctctgctctcctcacggccacgtttgcccatgcggattcagccctccgaatgccatccacgggcagcgtcgccccctgcgtgccacctctctccacctctctcttgtccctctctgccaccgtccacgccgcagccgcagcagccaccgcggctgcacacagcttccctctgtccttcgcgggggcattccccatgcttcccccaaacgcagcagcagcagtggccgcggccacagccatcagcccgcccttgtcagtatcagccacgtccagtcctcagcagaccagcagtggaacaaacaataaaccttaccgaccctgggggacagaagttggagctttttaaatttttcttgaacttcttgcaatagtaactgaatgtcctccatttcagagtcagcttaaaacctctgcaccctgaaggtagccatacagatgccgacagatccacaaaggaacaataaagctatttgagacacaaacctcacgagtggaaatgtggtattctcttttttttctctcccttttttgtttggttcaaggcagctcggtaactgacatcagcaacttttgaaaacttcacacttgttaccatttagaagtttcctggaaaatatatggaccgtaccatccagcagtgcatcagtatgtctgaattggggaagtaaaatgccctgactgaattctcttgagactagatgggacatacatatatagagagagagtgagagagtcgtgtttcgtaagtgcctgagcttaggaagttttcttctggatatataacattgcacaagggaagacgagtgtggaggataggttaagaaaggaaagggacagaagtcttgcaataggctgcagaca

SEQ ID NO: 24 - DNA, 1286 bp, Homo sapiens
ctccggtccccgccgctccccgtccccgctgctcctagcccctgccgcgtccccggcggagcgggcatggcgccacccgcggcgcctggccgggaccgtgtgggccgtgaggatgaggacggctgggagacgcgaggggaccgcaaggcccggaagcccctggtggagaagaagcggcgcgcgcggatcaacgagagcctgcaggagctgcggctgctgctggcgggcgccgaggccaagctggagaacgccgaagtgctggagctgacggtgcggcgggtccagggtgtgctgcggggccgggcgcgcgagcgcgagcagctgcaggcggaagcgagcgagcgcttcgctgccggctacatccagtgcatgcacgaggtgcacacgttcgtgtccacgtgccaggccatcgacgctaccgtcgctgccgagctcctgaaccatctgctcgagtccatgccgctgcgtgagggcagcagcttccaggatctgctgggggacgccctggcggggccacctagagcccctggacggagtggctggcctgcggggggcgctccgggatccccaatacccagccccccgggtcctggggacgacctgtgctccgacctggaggaggcccctgaggctgaactgagtcaggctcctgctgaggggcccgacttggtgcccgcagccctgggcagcctgaccacagcccaaattgcccggagtgtctggaggccttggtgaccaatgccagccagagtcctgcgggggtgggcccggccctccctggatctcctccctcctcccaggggttcagatgtggtggggtagggccctggaagtctcccaggtcttccctccctcctctgatggatggcttgcagggcagcccctggtaaccagcccagtcaggccccagccccgtttcttaagaaacttttagggaccctgcagctctggagtgggtggagggagggagctacgggcaggaggaagaattttgtagagctgccagcgctctcccaggttcacccacccaggcttcaccagccctgtgcgggctctgggggcagaggtggcagaaatggtgctgggcactagtgttccaggcagccctgggctaaacaaaagcttgaacttgccacttcagcggggagatgagaggcaggtgcactgagctgcactgcccagagctgtgatgctctgtacatcttgtttgtagcacacttgagtttgtgtattccattgacatcaaatgtgacaattttactaaataaagaattttggagttagtt

SEQ ID NO: 25 - DNA, 678 bp, Homo sapiens
atggtcacccgggatcgagctgagaatagggacggccccaagatgctcaagccgcttgtggagaagcggcgccgggaccgcatcaaccgcagcctggaagagctgaggctgctgctgctggagcggacccgggaccagaacctccggaacccgaagctggagaaagcggagatattggagttcgccgtgggctacttgagggagcgaagccgggtggagcccccgggggttccccggtccccagtccaggacgccaaggcgctcgccagctgctacttgtccggtttccgcgagtgcctgcttcgcttggcggccatcgcgcacgacgccagcccgaccgcccgcgcccagctcttctccgcgctgcacggctatctgcgccccaaaccgccccggcccaagccggtagatccgaggcctccagcgccgcgcccatccctggaccccgccgcaccggcccttggccctgcgctgcaccagcgccccccagtgcaccagggccaccctagcccgcgctgcgcatggtccccatccctctgctccccgcgcgccggggattctggcgcgccggcgcccctcaccggactgctgccgccgccaccgccgcctcacagacaagacggggcgcccaaggccccgctgcccccgccgcccgctttctggagaccttggccctga

SEQ ID NO: 26 - DNA, 1307 bp, Homo sapiens
cagcggtgcgagctccaggcccatgcactgaggaggcggaaacaaggggagcccccagagctccatcaagccccctccaaaggctcccctacccggtccacgccccccaccccccctccccgcctcctcccaattgtgcatttttgcagccggaggcggctccgagatggggctgtgagcttcgcccggggagggggaaagagcagcgaggagtgaagcgggggggtggggtgaagggtttggatttcggggcagggggcgcacccccgtcagcaggccctccccaaggggctcggaactctacctcttcacccacgcccctggtgcgctttgccgaaggaaagaataagaacagagaaggaggagggggaaaggaggaaaagggggaccccccaactggggggggtgaaggagagaagtagcaggaccagaggggaaggggctgctgcttgcatcagcccacaccatgctgaccccgccgttgctcctgctgctgcccctgctctcagctctggtcgcggcggctatcgacgcccctaagacttgcagccccaagcagtttgcctgcagagatcaaataacctgtatctcaaagggctggcggtgcgacggtgagagggactgcccagacggatctgacgaggcccctgagatttgtccacagagtaaggcccagcgatgccagccaaacgagcataactgcctgggtactgagctgtgtgttcccatgtcccgcctctgcaatggggtccaggactgcatggacggctcagatgaggggccccactgccgagagctccaaggcaactgctctcgcctgggctgccagcaccattgtgtccccacactcgatgggcccacctgctactgcaacagcagctttcagcttcaggcagatggcaagacctgcaaagattttgatgagtgctcagtgtacggcacctgcagccagctatgcaccaacacagacggctccttcatatgtggctgtgttgaaggatacctcctgcagccggataaccgctcctgcaaggccaagaacgagccagtagaccggccccctgtgctgttgatagccaactcccagaacatcttggccacgtacctgagtggggcccaggtgtctaccatcacacctacgagcacgcggcagaccacagccatggacttcagctatgccaacgagaccgtatgctgggtgcatgttggggacagtgctgctcagacgcagctcaagtgtgcccgcatgcctggcctaaagggcttcgtggatgagcacaccatcaacatctccctcagtctgcacc

SEQ ID NO: 27 - DNA, 1307 bp, Homo sapiens; identical to SEQ ID NO: 26

SEQ ID NO: 28 - DNA, 1307 bp, Homo sapiens; identical to SEQ ID NO: 26

SEQ ID NO: 29 - DNA, 4700 bp, Homo sapiens
caggcatgaacagtttcctcatcttcgccaggaggatagacattcgcatggtctccctggacatcccttattttgctgatgtggtggtaccaatcaacattaccatgaagaacaccattgccattggagtagacccccaggaaggaaaggtgtactggtctgacagcacactgcacaggatcagtcgtgccaatctggatggctcacagcatgaggacatcatcaccacagggctacagaccacagatgggctcgcggttgatgccattggccggaaagtatactggacagacacgggaacaaaccggattgaagtgggcaacctggacgggtccatgcggaaagtgttggtgtggcagaaccttgacagtccccgggccatcgtactgtaccatgagatggggtttatgtactggacagactggggggagaatgccaagttagagcggtccggaatggatggctcagaccgcgcggtgctcatcaacaacaacctaggatggcccaatggactgactgtggacaaggccagctcccaactgctatgggccgatgcccacaccgagcgaattgaggctgctgacctgaatggtgccaatcggcatacattggtgtcaccggtgcagcacccatatggcctcaccctgctcgactcctatatctactggactgactggcagactcggagcatccaccgtgctgacaagggtactggcagcaatgtcatcctcgtgaggtccaacctgccaggcctcatggacatgcaggctgtggaccgggcacagccactaggttttaacaagtgcggctcgagaaatggcggctgctcccacctctgcttgcctcggccttctggcttctcctgtgcctgccccactggcatccagctgaagggagatgggaagacctgtgatccctctcctgagacctacctgctcttctccagccgtggctccatccggcgtatctcactggacaccagtgaccacaccgatgtgcatgtccctgttcctgagctcaacaatgtcatctccctggactatgacagcgtggatggaaaggtctattacacagatgtgttcctggatgttatcaggcgagcagacctgaacggcagcaacatggagacagtgatcgggcgagggctgaagaccactgacgggctggcagtggactgggtggccaggaacctgtactggacagacacaggtcgaaataccattgaggcgtccaggctggatggttcctgccgcaaagtactgatcaacaatagcctggatgagccccgggccattgctgttttccccaggaaggggtacctcttctggacagactggggccacattgccaagatcgaacgggcaaacttggatggttctgagcggaaggtcctcatcaacacagacctgggttggcccaatggccttaccctggactatgatacccgcaggatctactgggtggatgcgcatctggaccggatcgagagtgctgacctcaatgggaaactgcggcaggtcttggtcagccatgtgtcccacccctttgccctcacacagcaagacaggtggatctactggacagactggcagaccaagtcaatccagcgtgttgacaaatactcaggccggaacaaggagacagtgctggcaaatgtggaaggactcatggatatcatcgtggtttcccctcagcggcagacagggaccaatgcctgtggtgtgaacaatggtggctgcacccacctctgctttgccagagcctcggacttcgtatgtgcctgtcctgacgaacctgatagccggccctgctcccttgtgcctggcctggtaccaccagctcctagggctactggcatgagtgaaaagagcccagtgctacccaacacaccacctaccaccttgtattcttcaaccacccggacccgcacgtctctggaggaggtggaaggaagatgctctgaaagggatgccaggctgggcctctgtgcacgttccaatgacgctgttcctgctgctccaggggaaggacttcatatcagctacgccattggtggactcctcagtattctgctgattttggtggtgattgcagctttgatgctgtacagacacaaaaaatccaagttcactgatcctggaatggggaacctcacctacagcaacccctcctaccgaacatccacacaggaagtgaagattgaagcaatccccaaaccagccatgtacaaccagctgtgctataagaaagagggagggcctgaccataactacaccaaggagaagatcaagatcgtagagggaatctgcctcctgtctggggatgatgctgagtgggatgacctcaagcaactgcgaagctcacgggggggcctcctccgggatcatgtatgcatgaagacagacacggtgtccatccaggccagctctggctccctggatgacacagagacggagcagctgttacaggaagagcagtctgagtgtagcagcgtccatactgcagccactccagaaagacgaggctctctgccagacacgggctggaaacatgaacgcaagctctcctcagagagccaggtctaaatgcccacattctcttccctgcctgcctgttccttctcctttatggacgtctagtccttgtgctcgcttacaccgcaggccccgcttctgtgtgcttgtcctcctcctcctcccaccccataactgttcctaagccttcaccggagctgtttaccacgtgagtccataactacctgtgcacaagaaatgatggcacatcacgagaatttagacctggattttaccatgaacctcacatcttgtactccatcctgggccccctgaaactgcttattcgtgattcctcaccagcgtagagctccacctcccctttccccagtaccctcagtgcctgcttctcagtgctgatgcagctgatgacccaggactgcgctctgccccatcacagccagcatgactgcttctctgagagaacttgcccatcaggggctgggacatgggggtgtgggtaaagacagggatgaaggatagaggctgagagaagaaggaagaatcagcccagcaggtatgggcatctgggaaacctccagcctcaagtgtgttagtaacatgaaaaagctttggggggtagttggatctgggtgtctggtccattgctggcagtggacattattcttgccctaagagacactgccttttcagcagcagatactggtgagatgggggtggctcaggctgttcttcctcctcctagaatgtctggagctgtttctacattcagataactgggtcccctatcacaaggctactggctaataggaattccctcctggtgccaccactggccagtacctttcctaagtctttgctcaaattaaccaggttgtgagccagtggcttgagtgaatgttaggccttgggggctgagtctctgaaaagtctaagaagctctgcctagaccaaatatggtatacctcctgacccctctctccctcatgtcctgggattctggggaagagacctagaaacaagctttcaaagaaaaaccagaagttgtcataaatggtcagaaagaacgatcaggttggagacttgggaaacccagggcctaaagagaagtatccatgagggtcaaacttcctgttgaacttcctatgttctttctcaagtgctcagggatctaagttagtggacagcaagcctgtggctacggggtggtgatgttcctcttccagctgtcccctcagctaaggggcttagtttccatgtgggatgccatcacttggttcatgctcattcacacaaagggcacgtgtctcagcctggtatcagggaaattgagacttatttttgccctaaaacgtctccctagctgttcttcgtggggtttttttgtttgtttttttgcctaatttgctttttctgaccaagccttgtggcaccagcaatctccaaagtcctgtggtgggagggctgaataaataaaaatacaaagaggtgggtaaggagtaggaaggtagagagcaccactgatgaggccctcctagcccatggcagacccagacctcttctcccccaggaattagaagtggcaggagagaacaacaggggctgggaatggaggggagaatttctaggggaagtttcctgagttgaaacttctcctgtggttactggtattgagaaatcagctaccaaagtgaaaaaggacaagatcaattcttttctagtcagttctaagactgctagagagagataccaggcccttagccttgctctcagtagcgtcagccccagttctgagcctccccacattacacttaacaagcagtaaaggagtgagcactttgggtccttagactcacgtctggggaggaagagcaagtagaaaagtggcattttcttgattggaaagggggaaggatcttattgcacttgggctgttcagaatgtagaaaggacatatttgaggaagtatctatttgagcactgatttactctgtaaaaagcaaaatctctctgtcctaaactaatggaagcgattctcccatgctcatgtgtaatggttttaacgttactcactggagagattggactttctggagttatttaaccactatgttcagtattttaggactttatgataatttaatataaatttagcttttcttaatc

SEQ ID NO: 30 - DNA, 5100 bp, Homo sapiens
atggagcccgagtgagcgcggcgcgggcccgtccggccgccggacaacatggaggcagcgccgcccgggccgccgtggccgctgctgctgctgctgctgctgctgctggcgctgtgcggctgcccggcccccgccgcggcctcgccgctcctgctatttgccaaccgccgggacgtacggctggtggacgccggcggagtcaagctggagtccaccatcgtggtcagcggcctggaggatgcggccgcagtggacttccagttttccaagggagccgtgtactggacagacgtgagcgaggaggccatcaagcagacctacctgaaccagacgggggccgccgtgcagaacgtggtcatctccggcctggtctctcccgacggcctcgcctgcgactgggtgggcaagaagctgtactggacggactcagagaccaaccgcatcgaggtggccaacctcaatggcacatcccggaaggtgctcttctggcaggaccttgaccagccgagggccatcgccttggaccccgctcacgggtacatgtactggacagactggggtgagacgccccggattgagcgggcagggatggatggcagcacccggaagatcattgtggactcggacatttactggcccaatggactgaccatcgacctggaggagcagaagctctactgggctgacgccaagctcagcttcatccaccgtgccaacctggacggctcgttccggcagaaggtggtggagggcagcctgacgcaccccttcgccctgacgctctccggggacactctgtactggacagactggcagacccgctccatccatgcctgcaacaagcgcactggggggaagaggaaggagatcctgagtgccctctactcacccatggacatccaggtgctgagccaggagcggcagcctttcttccacactcgctgtgaggaggacaatggcggctgctcccacctgtgcctgctgtccccaagcgagcctttctacacatgcgcctgccccacgggtgtgcagctgcaggacaacggcaggacgtgtaaggcaggagccgaggaggtgctgctgctggcccggcggacggacctacggaggatctcgctggacacgccggactttaccgacatcgtgctgcaggtggacgacatccggcacgccattgccatcgactacgacccgctagagggctatgtctactggacagatgacgaggtgcgggccatccgcagggcgtacctggacgggtctggggcgcagacgctggtcaacaccgagatcaacgaccccgatggcatcgcggtcgactgggtggcccgaaacctctactggaccgacacgggcacggaccgcatcgaggtgacgcgcctcaacggcacctcccgcaagatcctggtgtcggaggacctggacgagccccgagccatcgcactgcaccccgtgatgggcctcatgtactggacagactggggagagaaccctaaaatcgagtgtgccaacttggatgggcaggagcggcgtgtgctggtcaatgcctccctcgggtggcccaacggcctggccctggacctgcaggaggggaagctctactggggagacgccaagacagacaagatcgaggtgatcaatgttgatgggacgaagaggcggaccctcctggaggacaagctcccgcacattttcgggttcacgctgctgggggacttcatctactggactgactggcagcgccgcagcatcgagcgggtgcacaaggtcaaggccagccgggacgtcatcattgaccagctgcccgacctgatggggctcaaagctgtgaatgtggccaaggtcgtcggaaccaacccgtgtgcggacaggaacggggggtgcagccacctgtgcttcttcacaccccacgcaacccggtgtggctgccccatcggcctggagctgctgagtgacatgaagacctgcatcgtgcctgaggccttcttggtcttcaccagcagagccgccatccacaggatctccctcgagaccaataacaacgacgtggccatcccgctcacgggcgtcaaggaggcctcagccctggactttgatgtgtccaacaaccacatctactggacagacgtcagcctgaagaccatcagccgcgccttcatgaacgggagctcggtggagcacgtggtggagtttggccttgactaccccgagggcatggccgttgactggatgggcaagaacctctactgggccgacactgggaccaacagaatcgaagtggcgcggctggacgggcagttccggcaagtcctcgtgtggagggacttggacaacccgaggtcgctggccctggatcccaccaagggctacatctactggaccgagtggggcggcaagccgaggatcgtgcgggccttcatggacgggaccaactgcatgacgctggtggacaaggtgggccgggccaacgacctcaccattgactacgctgaccagcgcctctactggaccgacctggacaccaacatgatcgagtcgtccaacatgctgggtcaggagcgggtcgtgattgccgacgatctcccgcacccgttcggtctgacgcagtacagcgattatatctactggacagactggaatctgcacagcattgagcgggccgacaagactagcggccggaaccgcaccctcatccagggccacctggacttcgtgatggacatcctggtgttccactcctcccgccaggatggcctcaatgactgtatgcacaacaacgggcagtgtgggcagctgtgccttgccatccccggcggccaccgctgcggctgcgcctcacactacaccctggaccccagcagccgcaactgcagcccgcccaccaccttcttgctgttcagccagaaatctgccatcagtcggatgatcccggacgaccagcacagcccggatctcatcctgcccctgcatggactgaggaacgtcaaagccatcgactatgacccactggacaagttcatctactgggtggatgggcgccagaacatcaagcgagccaaggacgacgggacccagccctttgttttgacctctctgagccaaggccaaaacccagacaggcagccccacgacctcagcatcgacatctacagccggacactgttctggacgtgcgaggccaccaataccatcaacgtccacaggctgagcggggaagccatgggggtggtgctgcgtggggaccgcgacaagcccagggccatcgtcgtcaacgcggagcgagggtacctgtacttcaccaacatgcaggaccgggcagccaagatcgaacgcgcagccctggacggcaccgagcgcgaggtcctcttcaccaccggcctcatccgccctgtggccctggtggtagacaacacactgggcaagctgttctgggtggacgcggacctgaagcgcattgagagctgtgacctgtcaggggccaaccgcctgaccctggaggacgccaacatcgtgcagcctctgggcctgaccatccttggcaagcatctctactggatcgaccgccagcagcagatgatcgagcgtgtggagaagaccaccggggacaagcggactcgcatccagggccgtgtcgcccacctcactggcatccatgcagtggaggaagtcagcctggaggagttctcagcccacccatgtgcccgtgacaatggtggctgctcccacatctgtattgccaagggtgatgggacaccacggtgctcatgcccagtccacctcgtgctcctgcagaacctgctgacctgtggagagccgcccacctgctccccggaccagtttgcatgtgccacaggggagatcgactgtatccccggggcctggcgctgtgacggctttcccgagtgcgatgaccagagcgacgaggagggctgccccgtgtgctccgccgcccagttcccctgcgcgcggggtcagtgtgtggacctgcgcctgcgctgcgacggcgaggcagactgtcaggaccgctcagacgaggcggactgtgacgccatctgcctgcccaaccagttccggtgtgcgagcggccagtgtgtcctcatcaaacagcagtgcgactccttccccgactgtatcgacggctccgacgagctcatgtgtgaaatcaccaagccgccctcagacgacagcccggcccacagcagtgccatcgggcccgtcattggcatcatcctctctctcttcgtcatgggtggtgtctattttgtgtgccagcgcgtggtgtgccagcgctatgcgggggccaacgggcccttcccgcacgagtatgtcagcgggaccccgcacgtgcccctcaatttcatagccccgggcggttcccagcatggccccttcacaggcatcgcatgcggaaagtccatgatgagctccgtgagcctgatggggggccggggcggggtgcccctctacgaccggaaccacgtcacaggggcctcgtccagcagctcgtccagcacgaaggccacgctgtacccgccgatcctgaacccgccgccctccccggccacggacccctccctgtacaacatggacatgttctactcttcaaacattccggccactgtgagaccgtacaggccctacatcattcgaggaatggcgcccccgacgacgccctgcagcaccgacgtgtgtgacagcgactacagcgccagccgctggaaggccagcaagtactacctggatttgaactcggactcagacccctatccacccccacccacgccccacagccagtacctgtcggcggaggacagctgcccgccctcgcccgccaccgagaggagctacttccatctcttcccgccccctccgtccccctgcacggactcatcctgacctcggccgggccactctggcttctctgtgcccctgtaaatagttttaaatatgaacaaagaaaaaaatatattttatgatttaaaaaataaatataattgggattttaaaaacatgagaaatgtgaactgtgatggggtgggcagggctgggagaactttgtacagtggaacaaatatttataaacttaattttgtaaaacag

SEQ ID NO: 31 - DNA, 5301 bp, Homo sapiens
gcggccgccccggctcctcgcctcccccacttctggccacccctcgccggtgagagaagagaacgcgagaagggaagatgggggccgtcctgaggagcctcctggcctgcagcttctgtgtgctcctgagagcggcccctttgttgctttatgcaaacagacgggacttgcgattggttgatgctacaaatggcaaagagaatgctacgattgtagttggaggcttggaggatgcagctgcggtggactttgtgtttagtcatggcttgatatactggagtgatgtcagcgaagaagccattaaacgaacagaatttaacaaaactgagagtgtgcagaatgttgttgtttctggattattgtcccccgatgggctggcatgtgattggcttggagaaaaattgtactggacagattctgaaactaatcggattgaagtttctaatttagatggatctttacgaaaagttttattttggcaagagttggatcaacccagagctattgccttagatccttcaagtgggttcatgtactggacagactggggagaagtgccaaagatagaacgtgctggaatggatggttcaagtcgcttcattataataaacagtgaaatttactggccaaatggactgactttggattatgaagaacaaaagctttattgggcagatgcaaaacttaatttcatccacaaatcaaatctggatggaacaaatcggcaggcagtggttaaaggttcccttccacatccttttgccttgacgttatttgaggacatattgtactggactgactggagcacacactccattttggcttgcaacaagtatactggtgagggtctgcgtgaaatccattctgacatcttctctcccatggatatacatgccttcagccaacagaggcagccaaatgccacaaatccatgtggaattgacaatgggggttgttcccatttgtgtttgatgtctccagtcaagcctttttatcagtgtgcttgccccactggggtcaaactcctggagaatggaaaaacctgcaaagatggtgccacagaattattgcttttagctcgaaggacagacttgagacgcatttctttggatacaccagattttacagacattgttctgcagttagaagacatccgtcatgccattgccatagattacgatcctgtggaaggctacatctactggactgatgatgaagtgagggccatacgccgttcatttatagatggatctggcagtcagtttgtggtcactgctcaaattgcccatcctgatggtattgctgtggactgggttgcacgaaatctttattggacagacactggcactgatcgaatagaagtgacaaggctcaatgggaccatgaggaagatcttgatttcagaggacttagaggaaccccgggctattgtgttagatcccatggttgggtacatgtattggactgactggggagaaattccgaaaattgagcgagcagctctggatggttctgaccgtgtagtattggttaacacttctcttggttggccaaatggtttagccttggattatgatgaaggcaaaatatactggggagatgccaaaacagacaagattgaggttatgaatactgatggcactgggagacgagtactagtggaagacaaaattcctcacatatttggatttactttgttgggtgactatgtttactggactgactggcagaggcgtagcattgaaagagttcataaacgaagtgcagagagggaagtgatcatagatcagctgcctgacctcatgggcctaaaggctacaaatgttcatcgagtgattggttccaacccctgtgctgaggaaaacgggggatgtagccatctctgcctctatagacctcagggccttcgctgtgcttgccctattggctttgaactcatcagtgacatgaagacctgcattgtcccagaggctttccttttgttttcacggagagcagatatcagacgaatttctctggaaacaaacaataataatgtggctattccactcactggtgtcaaagaagcttctgctttggattttgatgtgacagacaaccgaatttattggactgatatatcactcaagaccatcagcagagcctttatgaatggcagtgcactggaacatgtggtagaattcggcttagattatccagaaggcatggcagtagactggcttgggaagaacttgtactgggcagacacaggaacgaatcgaattgaggtgtcaaagttggatgggcagcaccgacaagttttggtgtggaaagacctagatagtcccagagctctcgcgttggaccctgccgaaggatttatgtattggactgaatggggtggaaaacctaagatagacagagctgcaatggatggaagtgaacgtactaccttagttccaaatgtggggcgggcaaacggcctaactattgattatgctaaaaggaggctttattggacagacctggacaccaacttaatagaatcttcaaatatgcttgggctcaaccgtgaagttatagcagatgacttgcctcatccttttggcttaactcagtaccaagattatatctactggacggactggagccgacgcagcattgagcgtgccaacaaaaccagtggccaaaaccgcaccatcattcagggccatttggattatgtgatggacatcctcgtctttcactcatctcgacagtcagggtggaatgaatgtgcttccagcaatgggcactgctcccacctctgcttggctgtgccagttgggggttttgtttgtggatgccctgcccactactctcttaatgctgacaacaggacttgtagtgctcctacgactttcctgctcttcagtcaaaagagtgccatcaaccgcatggtgattgatgaacaacagagccccgacatcatccttcccatccacagccttcggaatgtccgggccattgactatgacccactggacaagcaactctattggattgactcacgacaaaacatgatccgaaaggcacaagaagatggcagccagggctttactgtggttgtgagctcagttccgagtcagaacctggaaatacaaccctatgacctcagcattgatatttacagccgctacatctactggacttgtgaggctaccaatgtcattaatgtgacaagattagatgggagatcagttggagtggtgctgaaaggcgagcaggacagacctcgagccattgtggtaaacccagagaaagggtatatgtattttaccaatcttcaggaaaggtctcctaaaattgaacgggctgctttggatgggacagaacgggaggtcctctttttcagtggcttaagtaaaccaattgctttagcccttgatagcaggctgggcaagctcttttgggctgattcagatctccggcgaattgaaagcagtgatctctcaggtgctaaccggatagtattagaagactccaatatcttgcagcctgtgggacttactgtgtttgaaaactggctctattggattgataaacagcagcaaatgattgaaaaaattgacatgacaggtcgagagggtagaaccaaagtccaagctcgaattgcccagcttagtgacattcatgcagtaaaggagctgaaccttcaagaatacagacagcacccttgtgctcaggataatggtggctgttcacatatttgtcttgtaaagggggatggtactacaaggtgttcttgccccatgcacctggttctacttcaagatgagctatcatgtggagaacctccaacatgttctcctcagcagtttacttgtttcacgggggaaattgactgtatccctgtggcttggcggtgcgatgggtttactgaatgtgaagaccacagtgatgaactcaattgtcctgtatgctcagagtcccagttccagtgtgccagtgggcagtgtattgatggtgccctccgatgcaatggagatgcaaactgccaggacaaatcagatgagaagaactgtgaagtgctttgtttaattgatcagttccgctgtgccaatggtcagtgcattggaaagcacaagaagtgtgatcataatgtggattgcagtgacaagtcagatgaactggattgttatccgactgaagaaccagcaccacaggccaccaatacagttggttctgttattggcgtaattgtcaccatttttgtgtctggaactgtatactttatctgccagaggatgttgtgtccacgtatgaagggagatggggaaactatgactaatgactatgtagttcatggaccagcttctgtgcctcttggttatgtgccacacccaagttctttgtcaggatctcttccaggaatgtctcgaggtaaatcaatgatcagctccctcagtatcatggggggaagcagtggacccccctatgaccgagcccatgttacaggagcatcatcaagtagttcttcaagcaccaaaggcacttacttccctgcaattttgaaccctccaccatccccagccacagagcgatcacattacactatggaatttggatattcttcaaacagtccttccactcataggtcatacagctacaggccatatagctaccggcactttgcaccccccaccacaccctgcagcacagatgtttgtgacagtgactatgctcctagtcggagaatgacctcagtggcaacagccaagggctataccagtgacttgaactatgattcagaacctgtgcccccacctcccacaccccgaagccaatacttgtcagcagaggagaactatgaaagctgcccaccttctccatacacagagaggagctattctcatcacctctacccaccgccaccctctccctgtacagactcctcctgaggaggggccctcctcctctgactgcctccaacgtaaaaatgtaaatataaatttggttgagatctggagggggggagggagctattagagaaggatgaggcagaccatgtacagttaaaattataaaatggggtagggaatactggagatatttgtacagaagaaaaggatatttatatattttcttaaaacagcagatttgctgcttgtgccataaaagtttgtataaaaaaaatttgtactaaaagttttatttttgcaaactaaatacacaaagcatgccttaaacccagtgaagcaactgagtacaaaggaaacaggaataataaaggcatcactgaccaggaatatctgggctttattgataccaaaaaaaaaaaaa

SEQ ID NO: 32 - DNA, 3941 bp, Homo sapiens
tgctgctgctgctgctgctgctgctgctgcagctccagcatcttgcggcggcagcggctgatccgctgctcggcggccaagggccggccaaggattgcgaaaaggaccaattccagtgccggaacgagcgctgcatcccctctgtgtggagatgcgacgaggacgatgactgcttagaccacagcgacgaggacgactgccccaagaagacctgtgcagacagtgacttcacctgtgacaacggccactgcatccacgaacggtggaagtgtgacggcgaggaggagtgtcctgatggctccgatgagtccgaggccacttgcaccaagcaggtgtgtcctgcagagaagctgagctgtggacccaccagccacaagtgtgtacctgcctcgtggcgctgcgacggggagaaggactgcgagggtggagccggatgaggccggctgtgctacctcactgggcacctgccgtggggacgagttccagtgtggggatgggacatgtgtccttgcaatcaagcactgcaaccaggagcaggactgtccagatgggagtgatgaagctggctgcctacaggggctgaacgagtgtctgcacaacaatggcggctgctcacacatctgcactgacctcaagattggctttgaatgcacgtgcccagcaggcttccagctcctggaccagaagacctgtggcgacattgatgagtgcaaggacccagatgcctgcagccagatctgtgtcaattacaagggctattttaagtgtgagtgctaccctggctacgagatggacctactgaccaagaactgcaaggctgctggtggcaagagcccatccctaatcttcaccaaccggcacgaggtgcggaggatcgacctggtgaagcggaactattcacgcctcatccccatgctcaagaatgtcgtggcactagatgtggaagttgccaccaatcgcatctactggtgtgacctctcctaccgtaagatctatagcgcctacatggacaaggccagtgacccgaaagagcaggaggtcctcattgacgagcagttgcactctccagagggcctggcagtggactgggtccacaagcacatctactggactgactcgggcaataagaccatctcagtggccacagttgatggtggccgccgacgcactctcttcagccgtaacctcagtgaaccccgggccatcgctgttgaccccctgcgagggttcatgtattggtctgactggggggaccaggccaagattgagaaatctgggctcaacggtgtggaccggcaaacactggtgtcagacaatattgaatggcccaacggaatcaccctggatctgctgagccagcgcttgtactgggtagactccaagctacaccaactgtccagcattgacttcagtggaggcaacagaaagacgctgatctcctccactgacttcctgagccacccttttgggatagctgtgtttgaggacaaggtgttctggacagacctggagaacgaggccattttcagtgcaaatcggctcaatggcctggaaatctccatcctggctgagaacctcaacaacccacatgacattgtcatcttccatgagctgaagcagccaagagctccagatgcctgtgagctgagtgtccagcctaatggaggctgtgaatacctgtgccttcctgctcctcagatctccagccactctcccaagtacacatgtgcctgtcctgacacaatgtggctgggtccagacatgaagaggtgctaccgagcacctcaatctacctcaactacgacgttagcttctaccatgacgaggacagtacctgccaccacaagagcccccgggaccaccgtccacagatccacctaccagaaccacagcacagagacaccaagcctgacagctgcagtcccaagctcagttagtgtccccagggctcccagcatcagcccgtctaccctaagccctgcaaccagcaaccactcccagcactatgcaaatgaagacagtaagatgggctcaacagtcactgccgctgttatcgggatcatcgtgcccatagtggtgatagccctcctgtgcatgagtggatacctgatctggagaaactggaagcggaagaacaccaaaagcatgaattttgacaacccagtctacaggaaaacaacagaagaagaagacgaagatgagctccatatagggagaactgctcagattggccatgtctatcctgcagcaatcagcagctttgatcgcccactgtgggcagagccctgtcttggggagaccagagaaccggaagacccagcccctgccctcaaggagctttttgtcttgccgggggaaccaaggtcacagctgcaccaactcccgaagaaccctctttccgagctgcctgtcgtcaaatccaagcgagtggcattaagccttgaagatgatggactaccctgaggatgggatcacccccttcgtgcctcatggaattcagtcccatgcactacactctggatggtgtatgactggatgaatgggtttctatatatgggtctgtgtgagtgtatgtgtgtgtgtgattttttttttaaatttatgttgcggaaaggtaaccacaaagttatgatgaactgcaaacatccaaaggatgtgagagtttttctatgtataatgttttatacactttttaactggttgcactacccatgaggaattcgtggaatggctactgctgactaacatgatgcacataaccaaatgggggccaatggcacagtaccttactcatcatttaaaaactatatttacagaagatgtttggttgctgggggggcttttttaggttttggggcatttgttttttgtaaataagatgattatgctttgtggctatccatcaacataagtaaaaaaaaaaaaaaaacacttcaactccctcccccatttagattatttattaacatattttaaaaatcagatgagttctataaataatttagagaagtgagagtatttatttttggcatgtttggcccaccacacagactctgtgtgtgtatgtgtgtgtttatatgtgtatgtgtgtgacagaaaaatctgtagagaagaggcacatctatggctactgttcaaatacataaagataaatttattttcacacagtccacaaggggtatatcttgtagttttcagaaaagcctttggaaatctggatcagaaaatagataccatggtttgtgcaattatgtagtaaaaaaggcaaatcttttcacctctggctattcctgagaccccaggaagtcaggaaaagcctttcagctcacccatggctgctgtgactcctaccagggctttcttggctttggcgaaggtcagtgtacagacattccatggtaccagagtgctcagaaactcaagataggatatgcctcaccctcagctactccttgttttaaagttcagctctttgagtaacttcttcaatttctttcaggacacttgggttgaattcagtaagtttcctctgaagcaccctgaagggtgccatccttacagagctaagtggagacgtttccagatcagcccaagtttactatagagactggcccaggcactgaatgtctaggacatgctgtggatgaagataaagatggtggaataggttttatcacatctcttatttctcttttccccttactctctaccatttcctttatgtggggaaacattttaaggtaataaataggttacttaccatcatatgttcatatagatgaaactaatttttggcttaagtcagaacaactggccaaaattgaagtcatatttgaggggggaaatggcatacgcaatattatattatattggatatttatgttcacacaggaatttggtttactgctttgtaaataaaaggaaaaactccgggtatatgt

SEQ ID NO: 33 - DNA, 1727 bp, Homo sapiens
gcaatggccacctttagccgccaggaatttttccagcagctactgcaaggctgtctcctgcctactgcccagcagggccttgaccagatctggctgctccttgccatctgcctcgcctgccgcctcctctggaggctcgggttgccatcctacctgaagcatgcaagcaccgtggcaggcgggttcttcagcctctaccacttcttccagctgcacatggtttgggtcgtgctgctcagcctcctgtgctacctcgtgctgttcctctgccgacattcctcccatcgaggcgtcttcctatccgtcaccatcctcatctacctactcatgggtgagatgcacatggtagacaccgtgacatggcacaagatgcgaggggcacagatgattgtggccatgaaggcagtgtctctgggcttcgacctggaccggggcgaggtgggtacggtgccctcgccagtggagttcatgggctacctctacttcgtgggcaccatcgtcttcgggccctggatatccttccacagctacctacaaactgtccaaggccgcccactgagctgccggtggctgcagaaggtggcccggagcctggcactggccctgctgtgccttgtgctgtccacttgcgtgggcccctacctcttcccgtacttcatccccctcaacggtgaccgcctccttcgcaacaagaaacgcaaagccaggggcaccatggtaaggtggctgcgagcctacgagagtgctgtctccttccacttcagcaactattttgtgggctttctttccgaggccacggccacgttggcgggggctggctttaccgaggagaaggatcacctggaatgggacctgacggtgtccaagccactgaatgtggagctgcctcggtcaatggtggaagttgtcacaagctggaacctgcccatgtcttattggctaaataactatgttttcaagaatgctctccgcctggggaccttctcggctgtgctggtcacctatgcagccagcgccctcctacatggcttcagtttccacctggctgcggtcctgctgtccctggcttttatcacttacgtggagcatgtcctccggaagcgcctggctcggatcctcagtgcctgtgtcttgtcaaagcggtgcccgccagactgttcgcaccagcatcgcttgggcctgggggtgcgagccttaaacttgctctttggagctctggccatcttccacctggcctacctgggctccctgtttgatgtcgatgtggatgacaccacagaggagcagggctacggcatggcatacactgtccacaagtggtcagagctcagctgggccagtcactgggtcacttttggatgctggatcttctaccgtctcataggctgaggcacatctgtggaccctcataaccctcttaagacccctctcagggtgccactgatgggggatgagggaaggccctctctctactccttgaccccctccatccttgacccccaacacctcaacacacacacacacacacacacacaaaatcacaccattttcatgcctgtcaatccccaccccaccacaaggcaggaagggggtggtgcctgctggggcctagagggggatgcttggggaaacagagaaggggagatccagggcctccccgccttccttcttcctttttatatacaatttgttattgtcaaataaaagtaggaaatattcaataggct

SEQ ID NO: 34 - DNA, 1475 bp, Homo sapiens
cggcacggtttcgtggggacccaggcttgcaaagtgacggtcattttctctttctttctccctcttgagtccttctgagatgatggctctgggcgcagcgggagctacccgggtctttgtcgcgatggtagcggcggctctcggcggccaccctctgctgggagtgagcgccaccttgaactcggttctcaattccaacgctatcaagaacctgcccccaccgctgggcggcgctgcggggcacccaggctctgcagtcagcgccgcgccgggaatcctgtacccgggcgggaataagtaccagaccattgacaactaccagccgtacccgtgcgcagaggacgaggagtgcggcactgatgagtactgcgctagtcccacccgcggaggggacgcaggcgtgcaaatctgtctcgcctgcaggaagcgccgaaaacgctgcatgcgtcacgctatgtgctgccccgggaattactgcaaaaatggaatatgtgtgtcttctgatcaaaatcatttccgaggagaaattgaggaaaccatcactgaaagctttggtaatgatcatagcaccttggatgggtattccagaagaaccaccttgtcttcaaaaatgtatcacaccaaaggacaagaaggttctgtttgtctccggtcatcagactgtgcctcaggattgtgttgtgctagacacttctggtccaagatctgtaaacctgtcctgaaagaaggtcaagtgtgtaccaagcataggagaaaaggctctcatggactagaaatattccagcgttgttactgtggagaaggtctgtcttgccggatacagaaagatcaccatcaagccagtaattcttctaggcttcacacttgtcagagacactaaaccagctatccaaatgcagtgaactccttttatataatagatgctatgaaaaccttttatgaccttcatcaactcaatcctaaggatatacaagttctgtggtttcagttaagcattccaataacaccttccaaaaacctggagtgtaagagctttgtttctttatggaactcccctgtgattgcagtaaattactgtattgtaaattctcagtgtggcacttacctgtaaatgcaatgaaacttttaattatttttctaaaggtgctgcactgcctatttttcctcttgttatgtaaatttttgtacacattgattgttatcttgactgacaaatattctatattgaactgaagtaaatcatttcagcttatagttcttaaaagcataaccctttaccccatttaattctagagtctagaacgcaaggatctcttggaatgacaaatgataggtacctaaaatgtaacatgaaaatactagcttattttctgaaatgtactatcttaatgcttaaattatatttccctttaggctgtgatagtttttgaaataaaatttaacatttaatatcatgaaatgttataagtagacat

SEQ ID NO: 35 - DNA, 1122 bp, Homo sapiens
gcgggtctcgcttgggttccgctaatttctgtcctgaggcgtgagactgagttcatagggtcctgggtccccgaaccaggaagggttgagggaacacaatctgcaagcccccgcgacccaagtgaggggccccgtgttggggtcctccctccctttgcattcccacccctccgggctttgcgtcttcctggggaccccctcgccgggagatggccgcgttgatgcggagcaaggattcgtcctgctgcctgctcctactggccgcggtgctgatggtggagagctcacagatcggcagttcgcgggccaaactcaactccatcaagtcctctctgggcggggagacgcctggtcaggccgccaatcgatctgcgggcatgtaccaaggactggcattcggcggcagtaagaagggcaaaaacctggggcaggcctacccttgtagcagtgataaggagtgtgaagttgggaggtattgccacagtccccaccaaggatcatcggcctgcatggtgtgtcggagaaaaaagaagcgctgccaccgagatggcatgtgctgccccagtacccgctgcaataatggcatctgtatcccagttactgaaagcatcttaacccctcacatcccggctctggatggtactcggcacagagatcgaaaccacggtcattactcaaaccatgacttgggatggcagaatctaggaagaccacacactaagatgtcacatataaaagggcatgaaggagacccctgcctacgatcatcagactgcattgaagggttttgctgtgctcgtcatttctggaccaaaatctgcaaaccagtgctccatcagggggaagtctgtaccaaacaacgcaagaagggttctcatgggctggaaattttccagcgttgcgactgtgcgaagggcctgtcttgcaaagtatggaaagatgccacctactcctccaaagccagactccatgtgtgtcagaaaatttgatcaccattgaggaacatcatcaattgcagactgtgaagttgtgtatttaatgcattatagcatggtggaaaataaggttcagatgcagaagaatggctaaaataagaaacgtgataagaatatagatgatcac

SEQ ID NO: 36 - DNA, 2124 bp, Homo sapiens
  misc_feature (1432)..(1432): unknown nucleotide
ctatcacaatgagaccaacacagacacgaaggttggaaataataccatccatgtgcaccgagaaattcacaagataaccaacaaccagactggacaaatggtcttttcagagacagttatcacatctgtgggagacgaagaaggcagaaggagccacgagtgcatcatcgacgaggactgtgggcccagcatgtactgccagtttgccagcttccagtacacctgccagccatgccggggccagaggatgctctgcacccgggacagtgagtgctgtggagaccagctgtgtgtctggggtcactgcaccaaaatggccaccaggggcagcaatgggaccatctgtgacaaccagagggactgccagccggggctgtgctgtgccttccagagaggcctgctgttccctgtgtgcacacccctgcccgtggagggcgagctttgccatgaccccgccagccggcttctggacctcatcacctgggagctagagcctgatggagccttggaccgatgcccttgtgccagtggcctcctctgccagccccacagccacagcctggtgtatgtgtgcaagccgaccttcgtggggagccgtgaccaagatggggagatcctgctgcccagagaggtccccgatgagtatgaagttggcagcttcatggaggaggtgcgccaggagctggaggacctggagaggagcctgactgaagagatggcgctgggggagcctgcggctgccgccgctgcactgctgggaggggaagagatttagatctggaccaggctgtgggtagatgtgcaatagaaatagctaatttatttccccaggtgtgtgctttaggcgtgggctgaccaggcttcttcctacatcttcttcccagtaagtttcccctctggcttgacagcatgaggtgttgtgcatttgttcagctcccccaggctgttctccaggcttcacagtctggtgcttgggagagtcaggcagggttaaactgcaggagcagtttgccacccctgtccagattattggctgctttgcctctaccagttggcagacagccgtttgttctacatggctttgataattgtttgaggggaggagatggaaacaatgtggagtctccctctgattggttttggggaaatgtggagaagagtgccctgctttgcaaacatcaacctggcaaaaatgcaacaaatgaattttccacgcagttctttccatgggcataggtaagctgtgccttcagctgttgcagatgaaatgttctgttcaccctgcattacatgtgtttattcatccagcagtgttgctcagctcctacctctgtgccagggcagcattttcatatccaagatcaattccctctctcagcacagcctggggagggggtcattgttctcctcgtccatcagggatctcagaggnctcagagactgcaagctgcttgcccaagtcacacagctagtgaagaccagagcagtttcatctggttgtgactctaagctcagtgctctctccactaccccacaccagccttggtgccaccaaaagtgctccccaaaaggaaggagaatgggatttttcttttgaggcatgcacatctggaattaaggtcaaactaattctcacatccctctaaaagtaaactactgttaggaacagcagtgttctcacagtgtggggcagccgtccttctaatgaagacaatgatattgacactgtccctctttggcagttgcattagtaactttgaaaggtatatgactgagcgtagcatacaggttaacctgcagaaacagtacttaggtaattgtagggcgaggattataaatgaaatttgcaaaatcacttagcagcaactgaagacaattatcaaccacgtggagaaaatcaaaccgagcagggctgtgtgaaacatggttgtaatatgcgactgcgaacactgaactctacgccactccacaaatgatgttttcaggtgtcatggactgttgccaccatgtattcatccagagttcttaaagtttaaagttgcacatgattgtataagcatgctttctttgagttttaaattatgtataaacataagttgcatttagaaatcaagcataaatcac

SEQ ID NO: 37 - DNA, 816 bp, Homo sapiens
agacgacgtgctgagctgccagcttagtggaagctctgctctgggtggagagcagcctcgctttggtgacgcacagtgctgggaccctccaggagccccgggattgaaggatggtggcggccgtcctgctggggctgagctggctctgctctcccctgggagctctggtcctggacttcaacaacatcaggagctctgctgacctgcatggggcccggaagggctcacagtgcctgtctgacacggactgcaataccagaaagttctgcctccagccccgcgatgagaagccgttctgtgctacatgtcgtgggttgcggaggaggtgccagcgagatgccatgtgctgccctgggacactctgtgtgaacgatgtttgtactacgatggaagatgcaaccccaatattagaaaggcagcttgatgagcaagatggcacacatgcagaaggaacaactgggcacccagtccaggaaaaccaacccaaaaggaagccaagtattaagaaatcacaaggcaggaagggacaagagggagaaagttgtctgagaacttttgactgtggccctggactttgctgtgctcgtcatttttggacgaaaatttgtaagccagtccttttggagggacaggtctgctccagaagagggcataaagacactgctcaagctccagaaatcttccagcgttgcgactgtggccctggactactgtgtcgaagccaattgaccagcaatcggcagcatgctcgattaagagtatgccaaaaaatagaaaagctataaatatttcaaaataaagaagaatccacattgc

SEQ ID NO: 38 - DNA, 1476 bp, Homo sapiens
acggggcctgggcggsaggggcggtggctggagctcggtaaagctcgtgggaccccattgggggaatttgatccaaggaagcggtgattgccgggggaggagaagctcccagatccttgtgtccacttgcagcgggggaggcggagacgcggagcgggccttttggcgtccactgcgcggctgcaccctgccccatcctgccgggatcatggtctgcggcagcccgggagggatgctgctgctgcgggccgggctgcttgccctggctgctctctgcctgctccgggtgcccggggctcgggctgcagcctgtgagcccgtccgcatccccctgtgcaagtccctgccctggaacatgactaagatgcccaaccacctgcaccacagcactcaggccaacgccatcctggccatcgagcagttcgaaggtctgctgggcacccactgcagccccgatctgctcttcttcctctgtgccatgtacgcgcccatctgcaccattgacttccagcacgagcccatcaacccctgtaagtctgtgtgcgagcgggcccggcagggctgtgagcccatactcatcaagtaccgccactcgtggccggagaacctggcctgcgaggagctgccagtgtacgacaggggcgtgtgcatctctcccgaggccatcgttactgcggacggagctgattttcctatggattctagtaacggaaactgtagaggggcaagcagtgaacgctgtaaatgtaagcctattagagctacacagaagacctatttccggaacaattacaactatgtcattcgggctaaagttaaagagataaagactaagtgccatgatgtgactgcagtagtggaggtgaaggagattctaaagtcctctctggtaaacattccacgggacactgtcaacctctataccagctctggctgcctctgccctccacttaatgttaatgaggaatatatcatcatgggctatgaagatgaggaacgttccagattactcttggtggaaggctctatagctgagaagtggaaggatcgactcggtaaaaaagttaagcgctgggatatgaagcttcgtcatcttggactcagtaaaagtgattctagcaatagtgattccactcagagtcagaagtctggcaggaactcgaacccccggcaagcacgcaactaaatcccgaaatacaaaaagtaacacagtggacttcctattaagacttacttgcattgctggactagcaaaggaaaattgcactattgcacatcatattctattgtttactataaaaatcatgtgataactgattattacttctgtttctcttttggtttctgcttctctcttctctcaacccctttgtaatggtttgggggcagactcttaagtatattgtgagttttctatttcactaatcatgagaaaaactgttcttttgcaataataataaattaaacatgctgtta

SEQ ID NO: 39 - DNA, 882 bp, Homo sapiens
  misc_feature (615)..(615): unknown nucleotide
gaattcgttcagcctggttaagtccaagctggctcattctgctcccccgggtcggagccccccggagctgcgcgcgggcttgcagcgcctcgcccgcgctgtcctcccggtgtcccgcttctccgcgccccagccgccggctgccagcttttcggggccccgagtcgcacccagcgaagagagcgggcccgggacaagctcgaactccggccgcctcgcccttaaccagctccgtccctctaccccctaggggtcgcgcccacgatgctgcagggccctggctcgctgctgctgctcttcctcgcctcgcactgctgcctgggctcggcgcgcgggctcttcctctttggccagcccgacttctcctacaagcgcagcaattgcaagcccatcccggccaacctgcagctgtgccacggcatcgaataccagaacatgcggctgcccaacctgctgggccacgagaccatgaaggaggtgctggagcaggccggcgcttggatcccgctggtcatgaagcagtgccacccggacaccaagaagttcctgtgctcgctcttcgcccccgtctgcctcgatgacctagacgagaccatccagccatgccactctcgntgcgtgcaggtgaaggatcgctgcgccccggtcatgtccgccttcccctggcccgacatgcttgagtgcgaccgtttcccccaggacaacgacctttgcatccccctcgctagcagcgaccacctcctgccagccaccgaggaagctccaaaggtatgtgaagcctgcaaaaataaaaatgatgatgacaacgacataatggaaacgctttgtaaaaatgattttgcactgaaaataaaagtgaaggagataacctacatcaaccgt

SEQ ID NO: 40 - DNA, 10363 bp, Homo sapiens
aggtccaagggtagccaaggatggctgcagcttcatatgatcagttgttaaagcaagttgaggcactgaagatggagaactcaaatcttcgacaagagctagaagataattccaatcatcttacaaaactggaaactgaggcatctaatatgaaggaagtacttaaacaactacaaggaagtattgaagatgaagctatggcttcttctggacagattgatttattagagcgtcttaaagagcttaacttagatagcagtaatttccctggagtaaaactgcggtcaaaaatgtccctccgttcttatggaagccgggaaggatctgtatcaagccgttctggagagtgcagtcctgttcctatgggttcatttccaagaagagggtttgtaaatggaagcagagaaagtactggatatttagaagaacttgagaaagagaggtcattgcttcttgctgatcttgacaaagaagaaaaggaaaaagactggtattacgctcaacttcagaatctcactaaaagaatagatagtcttcctttaactgaaaatttttccttacaaacagatatgaccagaaggcaattggaatatgaagcaaggcaaatcagagttgcgatggaagaacaactaggtacctgccaggatatggaaaaacgagcacagcgaagaatagccagaattcagcaaatcgaaaaggacatacttcgtatacgacagcttttacagtcccaagcaacagaagcagagaggtcatctcagaacaagcatgaaaccggctcacatgatgctgagcggcagaatgaaggtcaaggagtgggagaaatcaacatggcaacttctggtaatggtcagggttcaactacacgaatggaccatgaaacagccagtgttttgagttctagtagcacacactctgcacctcgaaggctgacaagtcatctgggaaccaaggtggaaatggtgtattcattgttgtcaatgcttggtactcatgataaggatgatatgtcgcgaactttgctagctatgtctagctcccaagacagctgtatatccatgcgacagtctggatgtcttcctctcctcatccagcttttacatggcaatgacaaagactctgtattgttgggaaattcccggggcagtaaagaggctcgggccagggccagtgcagcactccacaacatcattcactcacagcctgatgacaagagaggcaggcgtgaaatccgagtccttcatcttttggaacagatacgcgcttactgtgaaacctgttgggagtggcaggaagctcatgaaccaggcatggaccaggacaaaaatccaatgccagctcctgttgaacatcagatctgtcctgctgtgtgtgttctaatgaaactttcatttgatgaagagcatagacatgcaatgaatgaactagggggactacaggccattgcagaattattgcaagtggactgtgaaatgtatgggcttactaatgaccactacagtattacactaagacgatatgctggaatggctttgacaaacttgacttttggagatgtagccaacaaggctacgctatgctctatgaaaggctgcatgagagcacttgtggcccaactaaaatctgaaagtgaagacttacagcaggttattgcgagtgttttgaggaatttgtcttggcgagcagatgtaaatagtaaaaagacgttgcgagaagttggaagtgtgaaagcattgatggaatgtgctttagaagttaaaaaggaatcaaccctcaaaagcgtattgagtgccttatggaatttgtcagcacattgcactgagaataaagctgatatatgtgctgtagatggtgcacttgcatttttggttggcactcttacttaccggagccagacaaacactttagccattattgaaagtggaggtgggatattacggaatgtgtccagcttgatagctacaaatgaggaccacaggcaaatcctaagagagaacaactgtctacaaactttattacaacacttaaaatctcatagtttgacaatagtcagtaatgcatgtggaactttgtggaatctctcagcaagaaatcctaaagaccaggaagcattatgggacatgggggcagttagcatgctcaagaacctcattcattcaaagcacaaaatgattgctatgggaagtgctgcagctttaaggaatctcatggcaaataggcctgcgaagtacaaggatgccaatattatgtctcctggctcaagcttgccatctcttcatgttaggaaacaaaaagccctagaagcagaattagatgctcagcacttatcagaaacttttgacaatatagacaatttaagtcccaaggcatctcatcgtagtaagcagagacacaagcaaagtctctatggtgattatgtttttgacaccaatcgacatgatgataataggtcagacaattttaatactggcaacatgactgtcctttcaccatatttgaatactacagtgttacccagctcctcttcatcaagaggaagcttagatagttctcgttctgaaaaagatagaagtttggagagagaacgcggaattggtctaggcaactaccatccagcaacagaaaatccaggaacttcttcaaagcgaggtttgcagatctccaccactgcagcccagattgccaaagtcatggaagaagtgtcagccattcatacctctcaggaagacagaagttctgggtctaccactgaattacattgtgtgacagatgagagaaatgcacttagaagaagctctgctgcccatacacattcaaacacttacaatttcactaagtcggaaaattcaaataggacatgttctatgccttatgccaaattagaatacaagagatcttcaaatgatagtttaaatagtgtcagtagtagtgatggttatggtaaaagaggtcaaatgaaaccctcgattgaatcctattctgaagatgatgaaagtaagttttgcagttatggtcaatacccagccgacctagcccataaaatacatagtgcaaatcatatggatgataatgatggagaactagatacaccaataaattatagtcttaaatattcagatgagcagttgaactctggaaggcaaagtccttcacagaatgaaagatgggcaagacccaaacacataatagaagatgaaataaaacaaagtgagcaaagacaatcaaggaatcaaagtacaacttatcctgtttatactgagagcactgatgataaacacctcaagttccaaccacattttggacagcaggaatgtgtttctccatacaggtcacggggagccaatggttcagaaacaaatcgagtgggttctaatcatggaattaatcaaaatgtaagccagtctttgtgtcaagaagatgactatgaagatgataagcctaccaattatagtgaacgttactctgaagaagaacagcatgaagaagaagagagaccaacaaattatagcataaaatataatgaagagaaacgtcatgtggatcagcctattgattatagtttaaaatatgccacagatattccttcatcacagaaacagtcattttcattctcaaagagttcatctggacaaagcagtaaaaccgaacatatgtcttcaagcagtgagaatacgtccacaccttcatctaatgccaagaggcagaatcagctccatccaagttctgcacagagtagaagtggtcagcctcaaaaggctgccacttgcaaagtttcttctattaaccaagaaacaatacagacttattgtgtagaagatactccaatatgtttttcaagatgtagttcattatcatctttgtcatcagctgaagatgaaataggatgtaatcagacgacacaggaagcagattctgctaataccctgcaaatagcagaaataaaagaaaagattggaactaggtcagctgaagatcctgtgagcgaagttccagcagtgtcacagcaccctagaaccaaatccagcagactgcagggttctagtttatcttcagaatcagccaggcacaaagctgttgaattttcttcaggagcgaaatctccctccaaaagtggtgctcagacacccaaaagtccacctgaacactatgttcaggagaccccactcatgtttagcagatgtacttctgtcagttcacttgatagttttgagagtcgttcgattgccagctccgttcagagtgaaccatgcagtggaatggtaagtggcattataagccccagtgatcttccagatagccctggacaaaccatgccaccaagcagaagtaaaacacctccaccacctcctcaaacagctcaaaccaagcgagaagtacctaaaaataaagcacctactgctgaaaagagagagagtggacctaagcaagctgcagtaaatgctgcagttcagagggtccaggttcttccagatgctgatactttattacattttgccacggaaagtactccagatggattttcttgttcatccagcctgagtgctctgagcctcgatgagccatttatacagaaagatgtggaattaagaataatgcctccagttcaggaaaatgacaatgggaatgaaacagaatcagagcagcctaaagaatcaaatgaaaaccaagagaaagaggcagaaaaaactattgattctgaaaaggacctattagatgattcagatgatgatgatattgaaatactagaagaatgtattatttctgccatgccaacaaagtcatcacgtaaagcaaaaaagccagcccagactgcttcaaaattacctccacctgtggcaaggaaaccaagtcagctgcctgtgtacaaacttctaccatcacaaaacaggttgcaaccccaaaagcatgttagttttacaccgggggatgatatgccacgggtgtattgtgttgaagggacacctataaacttttccacagctacatctctaagtgatctaacaatcgaatcccctccaaatgagttagctgctggagaaggagttagaggaggggcacagtcaggtgaatttgaaaaacgagataccattcctacagaaggcagaagtacagatgaggctcaaggaggaaaaacctcatctgtaaccatacctgaattggatgacaataaagcagaggaaggtgatattcttgcagaatgcattaattctgctatgcccaaagggaaaagtcacaagcctttccgtgtgaaaaagataatggaccaggtccagcaagcatctgcgtcttcttctgcacccaacaaaaatcagttagatggtaagaaaaagaaaccaacttcaccagtaaaacctataccacaaaatactgaatataggacacgtgtaagaaaaaatgcagactcaaaaaataatttaaatgctgagagagttttctcagacaacaaagattcaaagaaacagaatttgaaaaataattccaaggtcttcaatgataagctcccaaataatgaagatagagtcagaggaagttttgcttttgattcacctcatcattacacgcctattgaaggaactccttactgtttttcacgaaatgattctttgagttctctagattttgatgatgatgatgttgacctttccagggaaaaggctgaattaagaaaggcaaaagaaaataaggaatcagaggctaaagttaccagccacacagaactaacctccaaccaacaatcagctaataagacacaagctattgcaaagcagccaataaatcgaggtcagcctaaacccatacttcagaaacaatccacttttccccagtcatccaaagacataccagacagaggggcagcaactgatgaaaagttacagaattttgctattgaaaatactccggtttgcttttctcataattcctctctgagttctctcagtgacattgaccaagaaaacaacaataaagaaaatgaacctatcaaagagactgagccccctgactcacagggagaaccaagtaaacctcaagcatcaggctatgctcctaaatcatttcatgttgaagataccccagtttgtttctcaagaaacagttctctcagttctcttagtattgactctgaagatgacctgttgcaggaatgtataagctccgcaatgccaaaaaagaaaaagccttcaagactcaagggtgataatgaaaaacatagtcccagaaatatgggtggcatattaggtgaagatctgacacttgatttgaaagatatacagagaccagattcagaacatggtctatcccctgattcagaaaattttgattggaaagctattcaggaaggtgcaaattccatagtaagtagtttacatcaagctgctgctgctgcatgtttatctagacaagcttcgtctgattcagattccatcctttccctgaaatcaggaatctctctgggatcaccatttcatcttacacctgatcaagaagaaaaaccctttacaagtaataaaggcccacgaattctaaaaccaggggagaaaagtacattggaaactaaaaagatagaatctgaaagtaaaggaatcaaaggaggaaaaaaagtttataaaagtttgattactggaaaagttcgatctaattcagaaatttcaggccaaatgaaacagccccttcaagcaaacatgccttcaatctctcgaggcaggacaatgattcatattccaggagttcgaaatagctcctcaagtacaagtcctgtttctaaaaaaggcccaccccttaagactccagcctccaaaagccctagtgaaggtcaaacagccaccacttctcctagaggagccaagccatctgtgaaatcagaattaagccctgttgccaggcagacatcccaaataggtgggtcaagtaaagcaccttctagatcaggatctagagattcgaccccttcaagacctgcccagcaaccattaagtagacctatacagtctcctggccgaaactcaatttcccctggtagaaatggaataagtcctcctaacaaattatctcaacttccaaggacatcatcccctagtactgcttcaactaagtcctcaggttctggaaaaatgtcatatacatctccaggtagacagatgagccaacagaaccttaccaaacaaacaggtttatccaagaatgccagtagtattccaagaagtgagtctgcctccaaaggactaaatcagatgaataatggtaatggagccaataaaaaggtagaactttctagaatgtcttcaactaaatcaagtggaagtgaatctgatagatcagaaagacctgtattagtacgccagtcaactttcatcaaagaagctccaagcccaaccttaagaagaaaattggaggaatctgcttcatttgaatctctttctccatcatctagaccagcttctcccactaggtcccaggcacaaactccagttttaagtccttcccttcctgatatgtctctatccacacattcgtctgttcaggctggtggatggcgaaaactcccacctaatctcagtcccactatagagtataatgatggaagaccagcaaagcgccatgatattgcacggtctcattctgaaagtccttctagacttccaatcaataggtcaggaacctggaaacgtgagcacagcaaacattcatcatcccttcctcgagtaagcacttggagaagaactggaagttcatcttcaattctttctgcttcatcagaatccagtgaaaaagcaaaaagtgaggatgaaaaacatgtgaactctatttcaggaaccaaacaaagtaaagaaaaccaagtatccgcaaaaggaacatggagaaaaataaaagaaaatgaattttctcccacaaatagtacttctcagaccgtttcctcaggtgctacaaatggtgctgaatcaaagactctaatttatcaaatggcacctgctgtttctaaaacagaggatgtttgggtgagaattgaggactgtcccattaacaatcctagatctggaagatctcccacaggtaatactcccccggtgattgacagtgtttcagaaaaggcaaatccaaacattaaagattcaaaagataatcaggcaaaacaaaatgtgggtaatggcagtgttcccatgcgtaccgtgggtttggaaaatcgcctgaactcctttattcaggtggatgcccctgaccaaaaaggaactgagataaaaccaggacaaaataatcctgtccctgtatcagagactaatgaaagttctatagtggaacgtaccccattcagttctagcagctcaagcaaacacagttcacctagtgggactgttgctgccagagtgactccttttaattacaacccaagccctaggaaaagcagcgcagatagcacttcagctcggccatctcagatcccaactccagtgaataacaacacaaagaagcgagattccaaaactgacagcacagaatccagtggaacccaaagtcctaagcgccattctgggtcttaccttgtgacatctgtttaaaagagaggaagaatgaaactaagaaaattctatgttaattacaactgctatatagacattttgtttcaaatgaaactttaaaagactgaaaaattttgtaaataggtttgattcttgttagagggtttttgttctggaagccatatttgatagtatactttgtcttcactggtcttattttgggaggcactcttgatggttaggaaaaaaatagtaaagccaagtatgtttgtacagtatgttttacatgtatttaaagtagcatcccatcccaacttcctttaattattgcttgtcttaaaataatgaacactacagatagaaaatatgatatattgctgttatcaatcatttctagattataaactgactaaacttacatcagggaaaaattggtatttatgcaaaaaaaaatgtttttgtccttgtgagtccatctaacatcataattaatcatgtggctgtgaaattcacagtaatatggttcccgatgaacaagtttacccagcctgctttgctttactgcatgaatgaaactgatggttcaatttcagaagtaatgattaacagttatgtggtcacatgatgtgcatagagatagctacagtgtaataatttacactattttgtgctccaaacaaaacaaaaatctgtgtaactgtaaaacattgaatgaaactattttacctgaactagattttatctgaaagtaggtagaatttttgctatgctgtaatttgttgtatattctggtatttgaggtgagatggctgctcttttattaatgagacatgaattgtgtctcaacagaaactaaatgaacatttcagaataaattattgctgtatgtaaactgttactgaaattggtatttgtttgaagggtcttgtttcacatttgtattaataattgtttaaaatgcctcttttaaaagcttatataaatttttttcttcagcttctatgcattaagagtaaaattcctcttactgtaataaaaacaattgaagaagactgttgccacttaaccattccatgcgttggcacttatctattcctgaaatttcttttatgtgattagctcatcttgatttttaatatttttccacttaaacttttttttcttactccactggagctcagtaaaagtaaattcatgtaatagcaatgcaagcagcctagcacagactaagcattgagcataataggcccacataatttcctctttcttaatattatagaattctgtacttgaaattgattcttagacattgcagtctcttcgaggctttacagtgtaaactgtcttgccccttcatcttcttgttgcaactgggtctgacatgaacactttttatcaccctgtatgttagggcaagatctcagcagtgaagtataatcagcactttgccatgctcagaaaattcaaatcacatggaactttagaggtagatttaatacgattaagatattcagaagtatattttagaatccctgcctgttaaggaaactttatttgtggtaggtacagttctggggtacatgttaagtgtccccttatacagtggagggaagtcttccttcctgaaggaaaataaactgacacttattaactaagataatttacttaatatatcttccctgatttgttttaaaagatcagagggtgactgatgatacatgcatacatatttgttgaataaatgaaaatttatttttagtgataagattcatacactctgtatttggggagggaaaacctttttaagcatggtggggcactcagataggagtgaatacacctacctggtg

SEQ ID NO: 41 - DNA, 3381 bp, Homo sapiens
gggcccggggtcccgccaccaccgcgcgcgggacagattgattcactttggagctgtaagtactgatgtattagggtgcagcgctcattgttccttgacgcagagtcccaaaatgaatatccaagagcagggtttccccttggacctcggagcaagtttcaccgaagatgctccccgacccccagtgcctggtgaggagggagaactggtgtccacagacccgaggcccgccagctacagtttctgctccgggaaaggtgttggcattaaaggtgagacttcgacggccactccgaggcgctcggatctggacctggggtatgagcctgagggcagtgcctcccccaccccaccatacttgaagtgggctgagtcactgcattccctgctggatgaccaagatgggataagcctgttcaggactttcctgaagcaggagggctgtgccgacttgctggacttctggtttgcctgcactggcttcaggaagctggagccctgtgactcgaacgaggagaagaggctgaagctggcgagagccatctaccgaaagtacattcttgataacaatggcatcgtgtcccggcagaccaagccagccaccaagagcttcataaagggctgcatcatgaagcagctgatcgatcctgccatgtttgaccaggcccagaccgaaatccaggccactatggaggaaaacacctatccctccttccttaagtctgatatttatttggaatatacgaggacaggctcggagagccccaaagtctgtagtgaccagagctctgggtcagggacagggaagggcatatctggatacctgccgaccttaaatgaagatgaggaatggaagtgtgaccaggacatggatgaggacgatggcagagacgctgctccccccggaagactccctcagaagctgctcctggagacagctgccccgagggtctcctccagtagacggtacagcgaaggcagagagttcaggtatggatcctggcgggagccagtcaacccctattatgtcaatgccggctatgccctggccccagccaccagtgccaacgacagcgagcagcagagcctgtccagcgatgcagacaccctgtccctcacggacagcagcgtggatgggatccccccatacaggatccgtaagcagcaccgcagggagatgcaggagagcgtgcaggtcaatgggcgggtgcccctacctcacattcccgtaagtaccgggtgccgaaggaggtccgcgtggagcctcagaagttcgcggaggagctcatccaccgcctggaggctgtgcagcgcacgcgggaggccgaggagaagctggaggagcggctgaagcgcgtgcgcatggaggaggaaggtgaggacggcgatccatcgtcagggcccccagggccgtgtcacaagctgcctcccgcccccgcttggcaccacttcccgccccgctgtgtggacatgggctgtgccgggctccgggatgcacacgaggagaaccctgagagcatcctggacgagcacgtacagcgtgtgctgaggacacctggccgccagtcgcctgggcctggccatcgctccccggacagtgggcacgtggccaagatgccagtggcactggggggtgccgcctcggggcacgggaagcacgtacccaagtcaggggcgaagctggacgcggccggcctgcaccaccaccgacacgtccaccaccacgtccaccacagcacagcccggcccaaggagcaggtggaggccgaggccacccgcagggcccagagcagcttcgcctggggcctggaaccacacagccatggggcaaggtcccgaggctactcagagagtgttggcgctgcccccaacgccagtgatggcctcgcccacagtgggaaggtgggcgtggcgtgcaaaagaaatgccaagaaggctgagtcggggaagagcgccagcaccgaggtgccaggtgcctcggaggatgcggagaagaaccagaaaatcatgcagtggatcattgagggggaaaaggagatcagcaggcaccgcaggaccggccacgggtcttcggggacgaggaagccacagccccatgagaactccagacccttgtcccttgagcacccctgggccggccctcagctccggacctccgtgcagccctcccacctcttcatccaagaccccaccatgccaccccacccagctcccaaccccctaacccagctggaggaggcgcgccgacgtctggaggaggaagaaaagagagccagccgagcaccctccaagcagaggtatgtgcaggaggttatgcggcggggacgcgcctgcgtcaggccagcgtgcgcgccggtgctgcacgtggtaccagccgtgtcggacatggagctctccgagacagagacaagatcgcagaggaaggtgggcggcgggagtgcccagccgtgtgacagcatcgttgtggcgtactacttctgcggggaacccatcccctaccgcaccctggtgaggggccgcgctgtcaccctgggccagttcaaggagctgctgaccaaaaagggcagctacagatactacttcaagaaagtgagcgacgagtttgactgtggggtggtgtttgaggaggttcgagaggacgaggccgtcctgcccgtctttgaggagaagatcatcggcaaagtggagaaggtggactgataggctggtgggctggccgctgtgccaggcgaggcccttggcgggcacgggtgtcacggccaggcagatgacctcgtactcaggagcccgatggggaacagtgttgggtgtaccacccatccctgtggtctacccgtgtctagaggcaggtagggggtccctccaagtggtccacaagcttctgtcctgcccccaaggaggcagcctggaccactcctcatagcaatacttggagggcccagcccaagtgaggcagccgaggtccctgctgccagcttcaggtgacccccccccatcccccggcacctcccttgggcacgtgtgctgggatctactttccctctgggatttgcccacgtacccaggtctgggtggggcccaggcccggatgcagaggcctgcagggcctctgtcaattgtacgcgccaccgagtgccttcaacacagcttgtctcttgcctgccactgtgtgaatcggcgacggagcactgcacctgcctccagccgccggctgtgcagtcctgggtcctcctttctgagggcccgtgtaaatatgtacatttctcaggctaggccagcaggggctgcccgagtctgtttttcatgcgatgacacttgtacaattaattatcttttcaaaggtacttggataataatgaaataaaactgtttttgaacctg

SEQ ID NO: 42 - DNA, 2538 bp, Homo sapiens
gttcagatgagcccctgctgacttgagagagacagagagaccacgccgattgctgagaggaactggaagaagaagagattccagactcagtgggaagagctccctcaccatgagtagcgctatgttggtgacttgcctcccggaccccagcagcagcttccgtgaggatgccccgcggcccccagtgccaggggaagaaggggagaccccaccgtgtcaccatggtgggcaagggccaggtcaccaaacccatgtctgtctcttccaacaccaggcggaacgaagatgggttggggagccggaggggcgggcatctccggattcccctctgacccggtggaccaagtccttacactccttattgggcgatcaagacggtgcttacctgttccgaactttcctggagagggagaaatgcgtggataccttagacttctggtttgcctgcaatggattcaggcagatgaacctgaaggataccaaaactttacgagtagccaaagcgatctacaaaaggtacattgagaacaacagcattgtctccaagcagctgaagcctgccaccaagacctacataagagatggcatcaagaagcagcagattgattccatcatgtttgaccaggcgcagaccgagatccagtcggtgatggaggaaaatgcctaccagatgtttttgacttctgatatatacctcgaatatgtgaggagtgggggagaaaacacagcttacatgagtaatgggggactcgggagcctaaaggtcgtgtgtggctatctccccaccttgaatgaagaagaggagtggacttgtgccgacttcaagtgcaaactttcgccaaccgtggttggcttgtccagcaaaactctgagggccacggcgagtgtgaggtccacggaaactgttgacagtggatacaggtccttcaagaggagcgatcctgttaatccttatcacataggttctggctatgtctttgcaccagccaccagcgccaacgacagtgagatatccagtgatgcgctgacggatgattccatgtccatgacggacagcagtgtagatggaattcctccttatcgtgtgggcagtaagaaacagctccagagagaaatgcatcgcagtgtgaaggccaatggcagagtgtctctacctcatttcccgagaacccaccgcctgcccaaggagatgacccccgtggaacccgccacctttgcagctgagctgatctcgaggctggaaaagctgaagctggagttggagagccgccacagcctggaggagcgcctgcagcagatccgagaggatgaagagagagagggctccgagctcacactcaattcgcgggagggggcgcccacgcagcaccccctctccctactgccctccggcagctacgaggaagacccgcagacgatactggacgatcacctgtccagggtcctcaagacccctggctgccagtctccgggcgtaggccgctatagccctcgctcccgctccccggaccaccaccaccaccaccattcgcagtaccactccctgctcccgcccggtggcaagctgcctcccgcggccgcctcgccgggcgcctgccccctcctcgggggcaaaggctttgtgaccaagcagacgacgaagcatgtccaccaccactacatccaccaccatgccgtccccaagaccaaggaggagatcgaggcggaggccacgcagcgggtgcactgcttctgccctgggggcagcgagtattactgctactcgaaatgcaaaagccactccaaggctccggaaaccatgcccagcgagcagtttggtgcccaaagcacaaaaaaggcctaccccttggagtctgcccgctcgtctccaggcgaacgagccagccggcaccatctgtgggggggcaacagcgggcacccccgcaccaccccccgtgcccacctgttcacccaggaccctgcgatgccttccctgaccccacccaacacgctggctcatctggaggaggcctgtcgcaggctagctgaggtgtcgaagcccccaaagcagcggtgctgtgtggccagtcagcagagggacaggaatcattcggccactgttcagacgggagccacacccttctccaatccaagcctggctccagaagatcacaaagagccaaagaaactggcaggtgtccacgcgctccaggccagtgagttggttgtcacttactttttctgtggggaagaaattccataccggaggatgctgaaggctcagagcttgaccctgggccactttaaagagcagctcagcaaaaagggaaattataggtattacttcaaaaaagcaagcgatgagtttgcctgtggagcggtgtttgaggagatctgggaggatgagacggtgctcccgatgtatgaaggccggattctgggcaaagtggagcggatcgattgagccctggggtctggctttggtgaactgttggagcccgaagctcttgtgaactgtcttggctgtgagcaactgcgacaaaacattttgaaggaaaa

SEQ ID NO: 43 - DNA, 2557 bp, Homo sapiens
attccggctcccgcggctgcaggcgcgcggctagagtgcctggcgggctccggcttccgcgtccgccccggccccggtccagacttagtcttcagctccgcgcccgctccgccgcggcccaccgcgcccgccggcagccgagcccccagcgacgcccgcacagctccgggtgcccagacagggggccatgccgtgccggagggaggaggaagaggaagccggcgaggaggcggagggggaggaagaggaggaggacagcttcctcctactgcagcagtcagtggcgctgggcagctcgggcgaggtggaccggctggtggcccagatcggcgagacgctgcagctggacgcggcgcagcacagcccggcctcgccgtgcgggcccccgggggcgccgctgcgggccccggggcccctggctgcggcggtgccggcggacaaggccaggtccccggcggtgccgctgctgctgccgcccgcgttggcggagactgtgggcccggcgccccctggggtcctgcgctgcgccctgggggaccgcggccgcgtgcggggccgcgctgcgccctactgcgtggccgagctcgccacaggccccagcgcgctgtccccactgccccctcaggccgaccttgatgggcctccgggagctggcaagcagggcatcccgcagccgctgtcgggtccgtgccggcgaggatggctccggggcgccgccgcctcccgccgcctgcagcagcgacgcgggtcccaaccagaaacccgcacaggcgacgacgacccgcaccggcttctgcagcagctagtgctctctggaaacctcatcaaggaggccgtgcgaaggcttcattcgcgacggctgcagttacgtgcaaagcttccccaacgcccgctcctgggacctctgtcggccccggtgcatgaacccccttcgcctcgcagccctcgcgcggcctgcagtgaccctggcgcctccgggagggcgcagctcagaactggcgacggcgttcttgtgcctggcagctaacacgcccggggtggccacagcgccagcctcagactggagggcaaggggttcccttgagggctgcagttctactcaggctggtggagaactctggcttttggaagcgagagtaaaaagctaatgacgaggaaccgaaaaatcgcgagtgtttcgcgggtaactggggttgagggccaaaatatttggaatgaaggactttggccctatttaaggcagattttacagagcgcacctcaaacgtacaagtcagtaggactccttatttggcgtgacccgacctggccgcggagcctgcatttcctcgcagcctctcagtgccctccagccccgcgaccatgtggccacaatccacgcttctccggatcgcggtgcgccggaaccacggaggatgatgccagttacttgctttaccttttcagggctggctcctgatccactttgggggaggagaacatgagtagataatttcagggtgcagcccaatctgccagacttaaaaaaaccatcttgtgtctttggaggtgctgcttaataccaaacatgcggtgccatgaagggaccctttgggggttgaataggagttaacccctgcgctctctttgcaactgtctctcttctcagagtggtgggggaaggctgtacgacacgggtggggaaaggaggtgggggcggggagtattgaatggtggtggaagggtagagaggcgcggagtgaaccccacgccctgtctaaagtgtattttcagagccggcccgcctctcctcggttcaaggtcactgtttcctgggcacgcactgggttgcgggacagagtagccaggttctgccggtgctcggagaagagcgcagtgttttgcaagtgctggagtctcctgaggacacgcgcgtcgccgccaccgcgggtgtgggaaagcgcggacgtgctgggcggctgtgcttcggtaggcgaccaccgcccctggccgcgctccgggctttcacggaaactcccgagaccgggccctgggttcctcctctcctactcggctctgcagtcctactcaagcgggtggctctgggatcctgggggcctgggttgggggctagggagacgccatgtgatggacactccagggacacacagcctagcacagcagcttataatgggctctccggggccatttgcaataacagctgcaattccctggatagacgagttgatttcctccctctgcccctcccccagccatgccagctggcctttgtaagtgcaggaaaccgagtagaaaatgtgaccctccaaatggagaagctgcaggctttgccattgtgaaccatggtgaagtgcttggaacatactgttcactcactctaaaggcgctgagactgtgctgttgttctcgtttttatagtcaatggcttgttcatcatccagatgtggctactgacatatctacacttcgcaccggagtgtctggaattgtggctatcctgattataggattttaacttaactgaaatgcctgctttgaataaatgtgttgggttttt

SEQ ID NO: 44 - DNA, 1231 bp, Homo sapiens
gaaggaaggaaaaggtgattcgcgaagagagtgatcatgtcagggcggcccagaaccacctcctttgcggagagctgcaagccggtgcagcagccttcagcttttggcagcatgaaagttagcagagacaaggacggcagcaaggtgacaacagtggtggcaactcctgggcagggtccagacaggccacaagaagtcagctatacagacactaaagtgattggaaatggatcatttggtgtggtatatcaagccaaactttgtgattcaggagaactggtcgccatcaagaaagtattgcaggacaagagatttaagaatcgagagctccagatcatgagaaagctagatcactgtaacatagtccgattgcgttatttcttctactccagtggtgagaagaaagatgaggtctatcttaatctggtgctggactatgttccggaaacagtatacagagttgccagacactatagtcgagccaaacagacgctccctgtgatttatgtcaagttgtatatgtatcagctgttccgaagtttagcctatatccattcctttggaatctgccatcgggatattaaaccgcagaacctcttgttggatcctgatactgctgtattaaaactctgtgactttggaagtgcaaagcagctggtccgaggagaacccaatgtttcgtatatctgttctcggtactatagggcaccagagttgatctttggagccactgattatacctctagtatagatgtatggtctgctggctgtgtgttggctgagctgttactaggacaaccaatatttccaggggatagtggtgtggatcagttggtagaaataatcaaggtcctgggaactccaacaagggagcaaatcagagaaatgaacccaaactacacagaatttaaattccctcaaattaaggcacatccttggactaaggtcttccgaccccgaactccaccggaggcaattgcactgtgtagccgtctgctggagtatacaccaactgcccgactaacaccactggaagcttgtgcacattcattttttgatgaattacgggacccaaatgtcaaactaccaaatgggcgagacacacctgcactcttcaacttcaccactcaagaactgtcaagtaatccacctctggctaccatccttattcctcctcatgctcggattcaagcagctgcttcaacccccacaaatgccacagcagcgtcag

SEQ ID NO: 45 - DNA, 2980 bp, Homo sapiens
  misc_feature (2974)..(2974): unknown nucleotide
ggccgctcctgctgctgctactgccgccgccgcagcggctgctcgggctgagcacgccccggaacaggccgccgcgcgctgcgcgccggacccgctgcccctgccggcccggccgggtcgggcggcccagggaccgacagacttgacaacggtgacagcactggggcggcaccttcctacttctgcccagccacagccctcccctcacagttgagcacctgtttgcctgaagttaatttccagaagcaggagtccccagagccaggcagggggatgaaccgcgagggagctcccgggaagagtccggaggagatgtacattcagcagaaggtccgagtgctgctcatgctgcggaagatgggatcaaacctgacagccagcgaggaggagttcctgcgcacctatgcaggggtggtcaacagccagctcagccagctgcctccgcactccatcgaccagggtgcagaggacgtggtgatggcgttttccaggtcggagacggaagaccggaggcagtagctgcaaagcccttggaacaccctggatgctgttgaagggccaagagatctgtgtggctcctgggccggctgaatggcagcagccccccttgccccacctcccccttccctacccaaccctgccctgccccaccccacctcacagctactcagtggggctggcatcaagggagacaccagtggtgcgtttataattggcttaaagggatggacttgtgattggctgcaggaagaaacttttttattttttaaatcttgaccaacagaaaccttttatttttatttctgactcttattttttaaaaaatttgcgcctcggtatctggcttccctggaagctctccgagctctggtgctttagttaggtcatttttttagaaatgtgaagaggtctgattggctgcttaaactggaaagggactgtgattggctggttaatgggaaacggtttttttctttggctgcaggtgttctgctgatatcaacagcttccctattttgaatgcagaaaacagggtctgggacattagtcgttatatttgacttgaaaagaaagaaaccaagtgcgctttgcaatatttattacacaaagaacttgctgctgccttcacatttggggtttgtgtttgattggctttcgatgcgtgtgtttggtttcccattggttcacctgtgactcctgttgccatggattcacccccctctgctgccggctctgggcctgagggtccacctggagagtacatttgctttaatgagtgcacctgcctccaccagcaaggggaccccgagaaccctgagcagggtccacagctggaaagttgggcccctgaggagctttgtgtcgtcttgaacgagcagcccagggcctagaggtaaccgttaggcgggatttatgtgcactgcctgcatgagctggcaaccagccagcgtcccttggtgagaaagggattgctgaggcaccgtccaggccccaccggccaggccgcgcccagcagaggcgtactacccagctctgtcctcttggccatccttctgtgtaccacttcctgaggcctcattttgggggtcatcttggaaaggggaggagcttctcccagtgtgagaccccaaagactctggaggtcatctggcggaggtctctgggagcccagaacccacataaaagccccagcttggctcacaaggcccaggagacctccagctaaacaccaacccctgacctaccccagccaggctcctacctgtctgctgccagcacagtaggtcccggccagctctggagttctctcatcggaggcccatgccctccactccactgcctttggaagggtctctctccaggtcagcctggaagggacagtatcgtttgtttatgaaatgccactgggacagctggctgggccttcaccaagcaagtcccttcagactggcccttaagccaaactcaggcccagaattgcagttcagaatggcagtcctggaggcagggggtgaggggcaggtctagtgttcctgcaccaaacctaagtccttccacctgccacccccttccctgggagggaggtggtcctcctatctccctggctcactggcaggtgtgggatctggggagagcggctggagaaagatgcagtcctcaggaagggggccgccaccctcccctatgctggtagatgctgaggcccctaggtgcccagggccagtgggaccctctcagaaccaaatctttcccctttctcggggcttggggctcgggccgtaggggctcctgagtgtcatgaagtgcacaggagccaaatgaccgagccctggagagccccatggtgggtaggtggttcgtgctgtgctctggcaccatcagcctgttccagaaggaggattcgagcatcaggctaagaccctgtgtcctccaccatgcactcacccctagccctggttagctgacagtcagctgtggggaacacagctacaaccctaccctggcagggacctgagagcatctcaggaggggcagcgcatgtgtgcatgtgctgtgtgagtgagcacacccgtgtgcacactcatacacatgtgcacacacacgcactctccccgctcaggggcctggaggtctggctgagcccctggggaaaggtgagttctttcatctccctcctccaggtcggagtgcctggagtcaggtgtcgaggccacattgctggctgccccctctttgtagctcctataaagggcccacacctggtggatacctggttgagcgtgtggtctctgccccagcctgtccttgtcacgatcacaggccttgcttttgtaacaatgatgaccccggcctgtctcatcttctgaagaggaaaagtcaaagtgttgctgtggctccatatttcaactaaaaatatatctgttggagaaagaaattaacaataaagaattttcataggttaaaaaaaaaaaanaaaaag

SEQ ID NO: 46 - DNA, 761 bp, Homo sapiens
ggcggcaggaccagcatgcaccaccgaaacgactcccagaggctggggaaagctggctgcccgccagagccgtcgttgcaaatggcaaatactaatttcctctccaccttatcccctgaacactgcagacctttggcgggggaatgcatgaacaagctcaaatgcggcgctgctgaagcagagataatgaatctccccgagcgcgtggggactttttccgctatcccggctttagggggcatctcattacctccaggggtcatcgtcatgacagcccttcactcccccgcagcagcctcagcagccgtcacagacagtgcgtttcaaattgccaatctggcagactgcccgcagaatcattcctcctcctcctcgtcctcctcagggggagctggcggagccaacccagccaagaagaagaggaaaaggtgtggggtctgcgtgccctgcaagaggctcatcaactgtggcgtctgcagcagttgcaggaaccgcaaaacgggacaccagatctgcaaatttagaaaatgtgaagagctaaagaaaaaacctggcacttcactagagagaacacctgttcccagcgctgaagcattccgatggttcttttaaagcagtagtatatcttattttcaaggcatttggaaatgaagggcaaactaatgtcttgttttaagaaactgcttagtccaccactgaagaaaatatccagaaattattttcattttatgtatagggatttcttcaaaaaaaaaaaaaaa

SEQ ID NO: 47 - DNA, 2905 bp, Homo sapiens
cctctgaaggttctagaatcgatagtgaattcgtgggacgggaagaggaagcactgcctttacttcagtgggaatctcggcctcagcctgcaagccaagtgttcacagtgaaaaaagcaagagaataagctaatactcctgtcctgaacaaggcagcggctccttggtaaagctactccttgatcgatcctttgcaccggattgttcaaagtggaccccaggggagaagtcggagcaaagaacttaccaccaagcagtccaagaggcccagaagcaaacctggaggtgagacccaaagaaagctggaaccatgctgactttgtacactgtgaggacacagagtctgttcctggaaagcccagtgtcaacgcagatgaggaagtcggaggtccccaaatctgccgtgtatgtggggacaaggccactggctatcacttcaatgtcatgacatgtgaaggatgcaagggctttttcaggagggccatgaaacgcaacgcccggctgaggtgccccttccggaagggcgcctgcgagatcacccggaagacccggcgacagtgccaggcctgccgcctgcgcaagtgcctggagagcggcatgaagaaggagatgatcatgtccgacgaggccgtggaggagaggcgggccttgatcaagcggaagaaaagtgaacggacagggactcagccactgggagtgcaggggctgacagaggagcagcggatgatgatcagggagctgatggacgctcagatgaaaacctttgacactaccttctcccatttcaagaatttccggctgccaggggtgcttagcagtggctgcgagttgccagagtctctgcaggccccatcgagggaagaagctgccaagtggagccaggtccggaaagatctgtgctctttgaaggtctctctgcagctgcggggggaggatggcagtgtctggaactacaaacccccagccgacagtggcgggaaagagatcttctccctgctgccccacatggctgacatgtcaacctacatgttcaaaggcatcatcagctttgccaaagtcatctcctacttcagggacttgcccatcgaggaccagatctccctgctgaagggggccgctttcgagctgtgtcaactgagattcaacacagtgttcaacgcggagactggaacctgggagtgtggccggctgtcctactgcttggaagacactgcaggtggcttccagcaacttctactggagcccatgctgaaattccactacatgctgaagaagctgcagctgcatgaggaggagtatgtgctgatgcaggccatctccctcttctccccagaccgcccaggtgtgctgcagcaccgcgtggtggaccagctgcaggagcaattcgccattactctgaagtcctacattgaatgcaatcggccccagcctgctcataggttcttgttcctgaagatcatggctatgctcaccgagctccgcagcatcaatgctcagcacacccagcggctgctgcgcatccaggacatacacccctttgctacgcccctcatgcaggagttgttcggcatcacaggtagctgagcggctgcccttgggtgacacctccgagaggcagccagacccagagccctctgagccgccactcccgggccaagacagatggacactgccaagagccgacaatgccctgctggcctgtctccctagggaattcctgctatgacagctggctagcattcctcaggaaggacatgggtgccccccacccccagttcagtctgtagggagtgaagccacagactcttacgtggagagtgcactgacctgtaggtcaggaccatcagagaggcaaggttgccctttccttttaaaaggccctgtggtctggggagaaatccctcagatcccactaaagtgtcaaggtgtggaagggaccaagcgaccaaggataggccatctggggtctatgcccacatacccacgtttgttcgcttcctgagtcttttcattgctacctctaatagtcctgtctcccacttcccactcgttcccctcctcttccgagctgctttgtgggctcaaggcctgtactcatcggcaggtgcatgagtatctgtgggagtcctctagagagatgagaagccaggaggcctgcaccaaatgtcagaagcttggcatgacctcattccggccacatcattctgtgtctctgcatccatttgaacacattattaagcactgataataggtagcctgctgtggggtatacagcattgactcagatatagatcctgagctcacagagtttatagttaaaaaaacaaacagaaacacaaacaatttggatcaaaaggagaaaatgataagtgacaaaagcagcacaaggaatttccctgtgtggatgctgagctgtgatggcaggcactgggtacccaagtgaaggttcccgaggacatgagtctgtaggagcaagggcacaaactgcagctgtgagtgcgtgtgtgtgatttggtgtaggtaggtctgtttgccacttgatggggcctgggtttgttcctggggctggaatgctgggtatgctctgtgacaaggctacgctgacaatcagttaaacacaccggagaagaaccatttacatgcaccttatatttctgtgtacacatctattctcaaagctaaagggtatgaaagtgcctgccttgtttatagccacttgtgagtaaaaatttttttgcattttcacaaattatactttatataaggcattccacacctaagaactagttttgggaaatgtagccctgggtttaatgtcaaatcaaggcaaaaggaattaaataatgtacttttggctaaaaaaaaaaaaaaaaaaaaaaaaaaaaaaaaaaaaaaaaaaaaaaaa

SEQ ID NO: 48 - DNA, 3089 bp, Homo sapiens
cccgcaggggttcctccaagatggcggcgcagaggaggagcttgctgcagagtgagcagcagccaagctggacagatgacctgcctctctgccacctctctggggttggctcagcctccaaccgcagctactctgctgatggcaagggcactgagagccacccgccagaggacagctggctcaagttcaggagtgagaacaactgcttcctgtatggggtcttcaacggctatgatggcaaccgagtgaccaacttcgtggcccagcggctgtccgcagagctcctgctgggccagctgaatgccgagcacgccgaggccgatgtgcggcgtgtgctgctgcaggccttcgatgtggtggagaggagcttcctggagtccattgacgacgccttggctgagaaggcaagcctccagtcgcaattgccagagggagtccctcagcaccagctgcctcctcagtatcagaagatccttgagagactcaagacgttagagagggaaatttcgggaggggccatggccgttgtggcggtccttctcaacaacaagctctacgtcgccaatgtcggtacaaaccgtgcacttttatgcaaatcgacagtggatgggttgcaggtgacacagctgaacgtggaccacaccacagagaacgaggatgagctcttccgtctttcgcagctgggcttggatgctggaaagatcaagcaggtggggatcatctgtgggcaggagagcacccggcggatcggggattacaaggttaaatatggctacacggacattgaccttctcagcgctgccaagtccaaaccaatcatcgcagagccagaaatccatggggcacagccgctggatggggtgacgggcttcttggtgctgatgtcggaggggttgtacaaggccctagaggcagcccatgggcctgggcaggccaaccaggagattgctgcgatgattgacactgagtttgccaagcagacctccctggacgcagtggcccaggccgtcgtggaccgggtgaagcgcatccacagcgacaccttcgccagtggtggggagcgtgccaggttctgcccccggcacgaggacatgaccctgctagtgaggaactttggctacccgctgggcgaaatgagccagcccacaccgagcccagccccagctgcaggaggacgagtgtaccctgtgtctgtgccatactccagcgcccagagcaccagcaagaccagcgtgaccctctcccttgtcatgccctcccagggccagatggtcaacggggctcacagtgcttccaccctggacgaagccacccccaccctcaccaaccaaagcccgaccttaaccctgcagtccaccaacacgcacacgcagagcagcagctccagctctgacggaggcctcttccgctcccggcccgcccactcgctcccgcctggcgaggacggtcgtgttgagccctatgtggactttgctgagttttaccgcctctggagcgtggaccatggcgagcagagcgtggtgacagcaccgtagggcagccggagaatgcagcccaagcagggcctggcatggggcaggacagggtccagccttttcctaacatctgcctgtgccacaacggccagcaggtgccccatcctctgcccacagcagactctgtcccatggctctccgggcagtagagtgtgtgagtgcagactggacctgtggttcataccttgtcaccacccgggaagctgaaggccacttcctcccagatggcctcagccaggaccatcgccctttctcagagcagagggccaggtatagaaaccgcagtgggcctgcaagccgcccgagcctccccagcagcctcctacagagcaggaagaggcgccctgtgaaccctgagtgttgcaggcccagcagaccctgctgtcccaagcccacccctcctcccaccatcacctccctcacctcgggacagtagccctccacttctccagcctctcagccctgtgctcctgcatccagagtggaacccaggctggtgtccgcatctgtccctgggccccacccctggacctgccttggttgtgtcatctgttgtaaacgttcaggaggaccagggcagcatctggggcctgggatggccacagaaggggcaggccaggtggaaaggagccagggggaagtggtctaagagacctggaactgccagaggatggcggcctgggcttccccagagccaggcgtgcgggagaggtgaggactggccccggtgggctgaggcaggggccgctgtcgtcaggcctgagccagggtgagctggtgcctgccttgcattttccttctggtgctgtgaagaccataggctggcaggcagctgagatgaactgtctttaccactgatgaggggcctctgccggctgagggtagcaagcaggggttgtgagtcaggctgggggacttgtttgaaagaaagaggagtggaaaatggttccaggagggaagaggttctttgagacacagtaccctgggaggcataggagaagggtcgggccagcccagcccagggcctgagttagactatttcccacatgttctctgccttcagtggggagggggtgccaccagggctgtcggccaggattgccactcctgtttcagaggaagcaggccgagagacttgcaccttggccaagccacacaatcagtggggcagccagagctcagacctgagccattttgtcagtatccaggaccccccggattctccacgccctccccatctcccagtctccctgccccccatgccccagaccggcccaccagggactagccgctgtcgcacagcctctggggtgcttggtctctgcgaagtcaaaggcctgacagctctgtggcctgggaatccattttcctgcggcagagcagggcctggtgtggaaccagggagctgtgggaagccacagcagaaatggaagaaaaacaggtctcagcccagggtcctcgctcactccctcactccccactttgaagccatctctgttctgcaggtgagaggatttaaagtcagtcacaaaggcttgggaacaaaaggaattc

SEQ ID NO: 49 - DNA, 3362 bp, Homo sapiens
aagcctctcggtctgtggcagcagcgttggcccggccccgggagcggagagcgaggggaggcggagacggaggaaggtctgaggagcagcttcagtccccgccgagccgccaccgcaggtcgaggacggtcggactcccgcggcgggaggagcctgttcccctgagggtatttgaagtataccatacaactgttttgaaaatccagcgtggacaatggctactcaagctgatttgatggagttggacatggccatggaaccagacagaaaagcggctgttagtcactggcagcaacagtcttacctggactctggaatccattctggtgccactaccacagctccttctctgagtggtaaaggcaatcctgaggaagaggatgtggatacctcccaagtcctgtatgagtgggaacagggattttctcagtccttcactcaagaacaagtagctgatattgatggacagtatgcaatgactcgagctcagagggtacgagctgctatgttccctgagacattagatgagggcatgcagatcccatctacacagtttgatgctgctcatcccactaatgtccagcgtttggctgaaccatcacagatgctgaaacatgcagttgtaaacttgattaactatcaagatgatgcagaacttgccacacgtgcaatccctgaactgacaaaactgctaaatgacgaggaccaggtggtggttaataaggctgcagttatggtccatcagctttctaaaaaggaagcttccagacacgctatcatgcgttctcctcagatggtgtctgctattgtacgtaccatgcagaatacaaatgatgtagaaacagctcgttgtaccgctgggaccttgcataacctttcccatcatcgtgagggcttactggccatctttaagtctggaggcattcctgccctggtgaaaatgcttggttcaccagtggattctgtgttgttttatgccattacaactctccacaaccttttattacatcaagaaggagctaaaatggcagtgcgtttagctggtgggctgcagaaaatggttgccttgctcaacaaaacaaatgttaaattcttggctattacgacagactgccttcaaattttagcttatggcaaccaagaaagcaagctcatcatactggctagtggtggaccccaagctttagtaaatataatgaggacctatacttacgaaaaactactgtggaccacaagcagagtgctgaaggtgctatctgtctgctctagtaataagccggctattgtagaagctggtggaatgcaagctttaggacttcacctgacagatccaagtcaacgtcttgttcagaactgtctttggactctcaggaatctttcagatgctgcaactaaacaggaagggatggaaggtctccttgggactcttgttcagcttctgggttcagatgatataaatgtggtcacctgtgcagctggaattctttctaacctcacttgcaataattataagaacaagatgatggtctgccaagtgggtggtatagaggctcttgtgcgtactgtccttcgggctggtgacagggaagacatcactgagcctgccatctgtgctcttcgtcatctgaccagccgacaccaagaagcagagatggcccagaatgcagttcgccttcactatggactaccagttgtggttaagctcttacacccaccatcccactggcctctgataaaggctactgttggattgattcgaaatcttgccctttgtcccgcaaatcatgcacctttgcgtgagcagggtgccattccacgactagttcagttgcttgttcgtgcacatcaggatacccagcgccgtacgtccatgggtgggacacagcagcaatttgtggagggggtccgcatggaagaaatagttgaaggttgtaccggagcccttcacatcctagctcgggatgttcacaaccgaattgttatcagaggactaaataccattccattgtttgtgcagctgctttattctcccattgaaaacatccaaagagtagctgcaggggtcctctgtgaacttgctcaggacaaggaagctgcagaagctattgaagctgagggagccacagctcctctgacagagttacttcactctaggaatgaaggtgtggcgacatatgcagctgctgttttgttccgaatgtctgaggacaagccacaagattacaagaaacggctttcagttgagctgaccagctctctcttcagaacagagccaatggcttggaatgagactgctgatcttggacttgatattggtgcccagggagaaccccttggatatcgccaggatgatcctagctatcgttcttttcactctggtggatatggccaggatgccttgggtatggaccccatgatggaacatgagatgggtggccaccaccctggtgctgactatccagttgatgggctgccagatctggggcatgcccaggacctcatggatgggctgcctccaggtgacagcaatcagctggcctggtttgatactgacctgtaaatcatcctttagctgtattgtctgaacttgcattgtgattggcctgtagagttgctgagagggctcgaggggtgggctggtatctcagaaagtgcctgacacactaaccaagctgagtttcctatgggaacaattgaagtaaactttttgttctggtcctttttggtcgaggagtaacaatacaaatggattttgggagtgactcaagaagtgaagaatgcacaagaatggatcacaagatggaatttagcaaaccctagccttgcttgttaaaatttttttttttttttttttaagaatatctgtaatggtactgactttgcttgctttgaagtagctctttttttttttttttttttttttttttgcagtaactgttttttaagtctctcgtagtgttaagttatagtgaatactgctacagcaatttctaatttttaagaattgagtaatggtgtagaacactaattaattcataatcactctaattaattgtaatctgaataaagtgtaacaattgtgtagcctttttgtataaaatagacaaatagaaaatggtccaattagtttcctttttaatatgcttaaaataagcaggtggatctatttcatgtttttgatcaaaaactatttgggatatgtatgggtagggtaaatcagtaagaggtgttatttggaaccttgttttggacagtttaccagttgccttttatcccaaagttgttgtaacctgctgtgatacgatgcttcaagagaaaatgcggttataaaaaatggttcagaattaaacttttaattcatt

SEQ ID NO: 50 - DNA, 1818 bp, Homo sapiens
atggacccggccgaggcggtgctgcaagagaaggcactcaagtttatgtgctctatgcccaggtctctgtggctgggctgctccagcctggcggacagcatgccttcgctgcgatgcctgtataacccagggactggcgcactcacagctttccagaattcctcagagagagaagactgtaataatggcgaaccccctaggaagataataccagagaagaattcacttagacagacatacaacagctgtgccagactctgcttaaaccaagaaacagtatgtttagcaagcactgctatgaagactgagaattgtgtggccaaaacaaaacttgccaatggcacttccagtatgattgtgcccaagcaacggaaactctcagcaagctatgaaaaggaaaaggaactgtgtgtcaaatactttgagcagtggtcagagtcagatcaagtggaatttgtggaacatcttatatcccaaatgtgtcattaccaacatgggcacataaactcgtatcttaaacctatgttgcagagagatttcataactgctctgccagctcggggattggatcatatcgctgagaacattctgtcatacctggatgccaaatcactatgtgctgctgaacttgtgtgcaaggaatggtaccgagtgacctctgatggcatgctgtggaagaagcttatcgagagaatggtcaggacagattctctgtggagaggcctggcagaacgaagaggatggggacagtatttattcaaaaacaaacctcctgacgggaatgctcctcccaactctttttatagagcactttatcctaaaattatacaagacattgagacaatagaatctaattggagatgtggaagacatagtttacagagaattcactgccgaagtgaaacaagcaaaggagtttactgtttacagtatgatgatcagaaaatagtaagcggccttcgagacaacacaatcaagatctgggataaaaacacattggaatgcaagcgaattctcacaggccatacaggttcagtcctctgtctccagtatgatgagagagtgatcataacaggatcatcggattccacggtcagagtgtgggatgtaaatacaggtgaaatgctaaacacgttgattcaccattgtgaagcagttctgcacttgcgtttcaataatggcatgatggtgacctgctccaaagatcgttccattgctgtatgggatatggcctccccaactgacattaccctccggagggtgctggtcggacaccgagctgctgtcaatgttgtagactttgatgacaagtacattgtttctgcatctggggatagaactataaaggtatggaacacaagtacttgtgaatttgtaaggaccttaaatggacacaaacgaggcattgcctgtttgcagtacagggacaggctggtagtgagtggctcatctgacaacactatcagattatgggacatagaatgtggtgcatgtttacgagtgttagaaggccatgaggaattggtgcgttgtattcgatttgataacaagaggatagtcagtggggcctatgatggaaaaattaaagtgtgggatcttgtggctgctttggacccccgtgctcctgcagggacactctgtctacggacccttgtggagcattccggaagagtttttcgactacagtttgatgaattccagattgtcagtagttcacatgatgacacaatcctcatctgggacttcctaaatgatccagctgcccaagctgaacccccccgttccccttctcgaacatacacctacatctccagataa

SEQ ID NO: 51 - DNA, 8694 bp, Homo sapiens
tgaggaatcaacagccgccatcttgtcgcggacccgaccggggcttcgagcgcgatctactcggccccgccggtcccgggccccacaaccgcccgcgctcgctcctctccctcgcagccggcagggcccccgacccccgtccgggccctcgccggcccggccgcccgtgcccggggctgttttcgcgagcaggtgaaaatggctgagaacttgctggacggaccgcccaaccccaaaagagccaaactcagctcgcccggtttctcggcgaatgacagcacagattttggatcattgtttgacttggaaaatgatcttcctgatgagctgatacccaatggaggagaattaggccttttaaacagtgggaaccttgttccagatgctgcttccaaacataaacaactgtcggagcttctacgaggaggcagcggctctagtatcaacccaggaataggaaatgtgagcgccagcagccccgtgcagcagggcctgggtggccaggctcaagggcagccgaacagtgctaacatggccagcctcagtgccatgggcaagagccctctgagccagggagattcttcagcccccagcctgcctaaacaggcagccagcacctctgggcccacccccgctgcctcccaagcactgaatccgcaagcacaaaagcaagtggggctggcgactagcagccctgccacgtcacagactggacctggtatctgcatgaatgctaactttaaccagacccacccaggcctcctcaatagtaactctggccatagcttaattaatcaggcttcacaagggcaggcgcaagtcatgaatggatctcttggggctgctggcagaggaaggggagctggaatgccgtaccctactccagccatgcagggcgcctcgagcagcgtgctggctgagaccctaacgcaggtttccccgcaaatgactggtcacgcgggactgaacaccgcacaggcaggaggcatggccaagatgggaataactgggaacacaagtccatttggacagccctttagtcaagctggagggcagccaatgggagccactggagtgaacccccagttagccagcaaacagagcatggtcaacagtttgcccaccttccctacagatatcaagaatacttcagtcaccaacgtgccaaatatgtctcagatgcaaacatcagtgggaattgtacccacacaagcaattgcaacaggccccactgcagatcctgaaaaacgcaaactgatacagcagcagctggttctactgcttcatgctcataagtgtcagagacgagagcaagcaaacggagaggttcgggcctgctcgctcccgcattgtcgaaccatgaaaaacgttttgaatcacatgacgcattgtcaggctgggaaagcctgccaagttgcccattgtgcatcttcacgacaaatcatctctcattggaagaactgcacacgacatgactgtcctgtttgcctccctttgaaaaatgccagtgacaagcgaaaccaacaaaccatcctggggtctccagctagtggaattcaaaacacaattggttctgttggcacagggcaacagaatgccacttctttaagtaacccaaatcccatagaccccagctccatgcagcgagcctatgctgctctcggactcccctacatgaaccagccccagacgcagctgcagcctcaggttcctggccagcaaccagcacagcctcaaacccaccagcagatgaggactctcaaccccctgggaaataatccaatgaacattccagcaggaggaataacaacagatcagcagcccccaaacttgatttcagaatcagctcttccgacttccctgggggccacaaacccactgatgaacgatggctccaactctggtaacattggaaccctcagcactataccaacagcagctcctccttctagcaccggtgtaaggaaaggctggcacgaacatgtcactcaggacctgcggagccatctagtgcataaactcgtccaagccatcttcccaacacctgatcccgcagctctaaaggatcgccgcatggaaaacctggtagcctatgctaagaaagtggaaggggacatgtacgagtctgccaacagcagggatgaatattatcacttattagcagagaaaatctacaagatacaaaaagaactagaagaaaaacggaggtcgcgtttacataaacaaggcatcttggggaaccagccagccttaccagccccgggggctcagccccctgtgattccacaggcacaacctgtgagacctccaaatggacccctgtccctgccagtgaatcgcatgcaagtttctcaagggatgaattcatttaaccccatgtccttggggaacgtccagttgccacaagcacccatgggacctcgtgcagcctccccaatgaaccactctgtccagatgaacagcatgggctcagtgccagggatggccatttctccttcccgaatgcctcagcctccgaacatgatgggtgcacacaccaacaacatgatggcccaggcgcccgctcagagccagtttctgccacagaaccagttcccgtcatccagcggggcgatgagtgtgggcatggggcagccgccagcccaaacaggcgtgtcacagggacaggtgcctggtgctgctcttcctaaccctctcaacatgctggggcctcaggccagccagctaccttgccctccagtgacacagtcaccactgcacccaacaccgcctcctgcttccacggctgctggcatgccatctctccagcacacgacaccacctgggatgactcctccccagccagcagctcccactcagccatcaactcctgtgtcgtcttccgggcagactcccaccccgactcctggctcagtgcccagtgctacccaaacccagagcacccctacagtccaggcagcagcccaggcccaggtgaccccgcagcctcaaaccccagttcagcccccgtctgtggctacccctcagtcatcgcagcaacagccgacgcctgtgcacgcccagcctcctggcacaccgctttcccaggcagcagccagcattgataacagagtccctaccccctcctcggtggccagcgcagaaaccaattcccagcagccaggacctgacgtacctgtgctggaaatgaagacggagacccaagcagaggacactgagcccgatcctggtgaatccaaaggggagcccaggtctgagatgatggaggaggatttgcaaggagcttcccaagttaaagaagaaacagacatagcagagcagaaatcagaaccaatggaagtggatgaaaagaaacctgaagtgaaagtagaagttaaagaggaagaagagagtagcagtaacggcacagcctctcagtcaacatctccttcgcagccgcgcaaaaaaatctttaaaccagaggagttacgccaggccctcatgccaaccctagaagcactgtatcgacaggacccagagtcattacctttccggcagcctgtagatccccagctcctcggaattccagactattttgacatcgtaaagaatcccatggacctctccaccatcaagcggaagctggacacagggcaataccaagagccctggcagtacgtggacgacgtctggctcatgttcaacaatgcctggctctataatcgcaagacatcccgagtctataagttttgcagtaagcttgcagaggtctttgagcaggaaattgaccctgtcatgcagtcccttggatattgctgtggacgcaagtatgagttttccccacagactttgtgctgctatgggaagcagctgtgtaccattcctcgcgatgctgcctactacagctatcagaataggtatcatttctgtgagaagtgtttcacagagatccagggcgagaatgtgaccctgggtgacgacccttcacagccccagacgacaatttcaaaggatcagtttgaaaagaagaaaaatgataccttagaccccgaacctttcgttgattgcaaggagtgtggccggaagatgcatcagatttgcgttctgcactatgacatcatttggccttcaggttttgtgtgcgacaactgcttgaagaaaactggcagacctcgaaaagaaaacaaattcagtgctaagaggctgcagaccacaagactgggaaaccacttggaagaccgagtgaacaaatttttgcggcgccagaatcaccctgaagccggggaggtttttgtccgagtggtggccagctcagacaagacggtggaggtcaagcccgggatgaagtcacggtttgtggattctggggaaatgtctgaatctttcccatatcgaaccaaagctctgtttgcttttgaggaaattgacggcgtggatgtctgcttttttggaatgcacgtccaagaatacggctctgattgcccccctccaaacacgaggcgtgtgtacatttcttatctggatagtattcatttcttccggccacgttgcctccgcacagccgtttaccatgagatccttattggatatttagagtatgtgaagaaattagggtatgtgacagggcacatctgggcctgtcctccaagtgaaggagatgattacatcttccattgccacccacctgatcaaaaaatacccaagccaaaacgactgcaggagtggtacaaaaagatgctggacaaggcgtttgcagagcggatcatccatgactacaaggatattttcaaacaagcaactgaagacaggctcaccagtgccaaggaactgccctattttgaaggtgatttctggcccaatgtgttagaagagagcattaaggaactagaacaagaagaagaggagaggaaaaaggaagagagcactgcagccagtgaaaccactgagggcagtcagggcgacagcaagaatgccaagaagaagaacaacaagaaaaccaacaagaacaaaagcagcatcagccgcgccaacaagaagaagcccagcatgcccaacgtgtccaatgacctgtcccagaagctgtatgccaccatggagaagcacaaggaggtcttcttcgtgatccacctgcacgctgggcctgtcatcaacaccctgccccccatcgtcgaccccgaccccctgctcagctgtgacctcatggatgggcgcgacgccttcctcaccctcgccagagacaagcactgggagttctcctccttgcgccgctccaagtggtccacgctctgcatgctggtggagctgcacacccagggccaggaccgctttgtctacacctgcaacgagtgcaagcaccacgtggagacgcgctggcactgcactgtgtgcgaggactacgacctctgcatcaactgctataacacgaagagccatgcccataagatggtgaagtgggggctgggcctggatgacgagggcagcagccagggcgagccacagtcaaagagcccccaggagtcacgccgggtgagcatccagcgctgcatccagtcgctggtgcacgcgtgccagtgccgcaacgccaactgctcgctgccatcctgccagaagatgaagcgggtggtgcagcacaccaagggctgcaaacgcaagaccaacgggggctgcccggtgtgcaagcagctcatcgccctctgctgctaccacgccaagcactgccaagaaaacaaatgccccgtgcccttctgcctcaacatcaaacacaagctccgccagcagcagatccagcaccgcctgcagcaggcccagctcatgcgccggcggatggccaccatgaacacccgcaacgtgcctcagcagagtctgccttctcctacctcagcaccgcccgggacccccacacagcagcccagcacaccccagacgccgcagccccctgcccagccccaaccctcacccgtgagcatgtcaccagctggcttccccagcgtggcccggactcagccccccaccacggtgtccacagggaagcctaccagccaggtgccggcccccccacccccggcccagccccctcctgcagcggtggaagcggctcggcagatcgagcgtgaggcccagcagcagcagcacctgtaccgggtgaacatcaacaacagcatgcccccaggacgcacgggcatggggaccccggggagccagatggcccccgtgagcctgaatgtgccccgacccaaccaggtgagcgggcccgtcatgcccagcatgcctcccgggcagtggcagcaggcgccccttccccagcagcagcccatgccaggcttgcccaggcctgtgatatccatgcaggcccaggcggccgtggctgggccccggatgcccagcgtgcagccacccaggagcatctcacccagcgctctgcaagacctgctgcggaccctgaagtcgcccagctcccctcagcagcaacagcaggtgctgaacattctcaaatcaaacccgcagctaatggcagctttcatcaaacagcgcacagccaagtacgtggccaatcagcccggcatgcagccccagcctggcctccagtcccagcccggcatgcaaccccagcctggcatgcaccagcagcccagcctgcagaacctgaatgccatgcaggctggcgtgccgcggcccggtgtgcctccacagcagcaggcgatgggaggcctgaacccccagggccaggccttgaacatcatgaacccaggacacaaccccaacatggcgagtatgaatccacagtaccgagaaatgttacggaggcagctgctgcagcagcagcagcaacagcagcagcaacaacagcagcaacagcagcagcagcaagggagtgccggcatggctgggggcatggcggggcacggccagttccagcagcctcaaggacccggaggctacccaccggccatgcagcagcagcagcgcatgcagcagcatctccccctccagggcagctccatgggccagatggcggctcagatgggacagcttggccagatggggcagccggggctgggggcagacagcacccccaacatccagcaagccctgcagcagcggattctgcagcaacagcagatgaagcagcagattgggtccccaggccagccgaaccccatgagcccccagcaacacatgctctcaggacagccacaggcctcgcatctccctggccagcagatcgccacgtcccttagtaaccaggtgcggtctccagcccctgtccagtctccacggccccagtcccagcctccacattccagcccgtcaccacggatacagccccagccttcgccacaccacgtctcaccccagactggttccccccaccccggactcgcagtcaccatggccagctccatagatcagggacacttggggaaccccgaacagagtgcaatgctcccccagctgaacacccccagcaggagtgcgctgtccagcgaactgtccctggtcggggacaccacgggggacacgctagagaagtttgtggagggcttgtagcattgtgagagcatcaccttttccctttcatgttcttggaccttttgtactgaaaatccaggcatctaggttctttttattcctagatggaactgcgacttccgagccatggaagggtggattgatgtttaaagaaacaatacaaagaatatatttttttgttaaaaaccagttgatttaaatatctggtctctctctttggtttttttttggcgggggggtggggggggttcttttttttccgttttgtttttgtttggggggaggggggttttgtttggattctttttgtcgtcattgctggtgactcatgcctttttttaacgggaaaaacaagttcattatattcatattttttatttgtattttcaagactttaaacatttatgtttaaaagtaagaagaaaaataatattcagaactgattcctgaaataatgcaagcttataatgtatcccgataactttgtgatgtttcgggaagatttttttctatagtgaactctgtgggcgtctcccagtattaccctggatgataggaattgactccggcgtgcacacacgtacacacccacacacatctatctatacataatggctgaagccaaacttgtcttgcagatgtagaaattgttgctttgtttctctgataaaactggttttagacaaaaaatagggatgatcactcttagaccatgctaatgttactagagaagaagccttcttttctttcttctatgtgaaacttgaaatgaggaaaagcaattctagtgtaaatcatgcaagcgctctaattcctataaatacgaaactcgagaagattcaatcactgtatagaatggtaaaataccaactcatttcttatatcatattgttaaataaactgtgtgcaacagacaaaaagggtggtccttcttgaattcatgtacatggtattaacacttagtgttcggggttttttgttatgaaaatgctgttttcaacattgtatttggactatgcatgtgttttttccccattgtatataaagtaccgcttaaaattgatataaattactgaggtttttaacatgtattctgttctttaagatcccctgtaagaatgtttaaggtttttatttatttatatatattttttggtctgttctttgtaaaaaaaaaaaaaaaaa

SEQ ID NO: 52 - DNA, 2109 bp, Homo sapiens
cgcgcagcgcgccggagtggtcggggcccgcggccgctcgcgcctctcgatgggcagctcgcacttgctcaacaagggcctgccgcttggcgtccgacctccgatcatgaacgggcccctgcacccgcggcccctggtggcattgctggatggccgggactgcacagtggagatgcccatcctgaaggacgtggccactgtggccttctgcgacgcgcagtccacgcaggagatccatgagaaggtcctgaacgaggctgtgggggccctgatgtaccacaccatcactctcaccagggaggacctggagaagttcaaagccctccgcatcatcgtccggattggcagtggttttgacaacatcgacatcaagtcggccggggatttaggcattgccgtctgcaacgtgcccgcggcgtctgtggaggagacggccgactcgacgctgtgccacatcctgaacctgtaccggcgggccacctggctgcaccaggcgctgcgggagggcacacgagtccagagcgtcgagcagatccgcgaggtggcgtccggcgctgccaggatccgcggggagaccttgggcatcatcggacttggtcgcgtggggcaggcagtggcgctgcgggccaaggccttcggcttcaacgtgctcttctacgacccttacttgtcggatggcgtggagcgggcgctggggctgcagcgtgtcagcaccctgcaggacctgctcttccacagcgactgcgtgaccctgcactgcggcctcaacgagcacaaccaccacctcatcaacgacttcaccgtcaagcagatgagacaaggggccttcctggtgaacacagcccggggtggcctggtggatgagaaggcgctggcccaggccctgaaggagggccggatccgcggcgcggccctggatgtgcacgagtcggaacccttcagctttagccagggccctctgaaggatgcacccaacctcatctgcaccccccatgctgcatggtacagcgagcaggcatccatcgagatgcgagaggaggcggcacgggagatccgcagagccatcacaggccggatcccagacagcctgaagaactgtgtcaacaaggaccatctgacagccgccacccactgggccagcatggaccccgccgtcgtgcaccctgagctcaatggggctgcctataggtaccctccgggcgtggtgggcgtggcccccactggcatcccagctgctgtggaaggtatcgtccccagcgccatgtccctgtcccacggcctgccccctgtggcccacccgccccacgccccttctcctggccaaaccgtcaagcccgaggcggatagagaccacgccagtgaccagttgtagcccgggaggagctctccagcctcggcgcctggggcagcgggcccggaaaccctcgaccagagtgtgtgagagcatgtgtgtggtggcccctggcactgcagagactggtccgggctgtcaggagggcgggagggcgcagcgctgggcctcgtgtcgcttgtcgtccgtcctgtgggcgctctgccctgtgtccttcgcgttcctcgttaagcagaagaagtcagtagttattctcccatgaacgttcttgtctgtgtacagtttttagaacattacaaaggatctgtttgcttagctgtcaacaaaaagaaaacctgaaggagcatttggaagtcaatttgaggtttttttttttggtttttttttttttgtattttggaacgtgccccagaatgaggcagttggcaaacttctcaggacaatgaatcttcccgtttttctttttatgccacacagtgcattgttttttctacctgcttgtcttatttttagcataatttagaaaaacaaaacaaaggctgtttttcctaattttggcatgaacccccccttgttccaaaatgaagacggcatcatcacgaagcagctccaaaaggaaaagcttggcaggtgccctcgtcctggggacgtggagggtggcacggtccccgcctgcaccagtgccgtcctgctgatgtggtaggctagcaatattttggttaaaatcatgtttgtggccgaacgggcccctgcacccg

SEQ ID NO: 53 - DNA, 2636 bp, Homo sapiens
agcaccataacatggtgtgggaagtgaagacaaatcagatgcctaatgcagtacagaaactcctgttggtgatggacaagagagcctcaggaatgaatgactcattggagttgctgcagtgtaatgagaatttgccatcttcacctggatataactcctgtgatgaacacatggagcttgatgaccttcctgaacttcaggcagttcaaagtgatcctacccaatctggcatgtaccagctgagttcagatgtttcacatcaagaatacccaagatcatcttggaaccaaaatacctcagacataccagaaactacttaccgtgaaaatgaggtggactggctaacagaattggcaaatatcgcgaccagtccacaaagtccactgatgcagtgctcattttacaatagatcatctcctgtacacatcatagccactagcaaaagtttacattcctatgcacgccctccaccagtgtcctcttcttcgaagagtgaaccagccttccctcatcaccattggaaggaggaaacaccagtaagacacgaaagggcaaatagtgagtcagaatctggcattttctgcatgtcctccctgtcagatgatgatgatttgggatggtgcaattcctggccttcaactgtctggcactgttttttgaaaggcacacgactgtgctttcataagggaagcaataaggaatggcaagatgttgaagattttgctagagctgaaggctgtgataatgaggaagatcttcaaatgggcattcacaagggctatggttctgatggtctaaagttgttatcacatgaagaaagtgtatcatttggcgagtctgtactgaagttgacttttgatcctggtacagtagaagatggtttacttaccgtagagtgtaagctggaccaccctttctatgttaaaaataaaggttggtcatcattttatccaagcttgactgtggtacagcatggcattccatgttgtgaagttcatattggcgatgtatgtctacctcctggacaccccgatgccattaattttgatgattcaggtgtttttgatacatttaaaagctatgacttcacacctatggattcttctgcagtttatgtgttaagtagtatggctcgccagcgtcgtgcatctttgtcttgtggaggacctggtggtcaagactttgcaagatctggattcagtaaaaactgtggctcacctggatcatcacagctctcttccaattctttgtatgctaaagctgtcaaaaaccacagctcagggactgtgagtgccacttctcctaataagtgcaaaagaccaatgaatgccttcatgctttttgccaaaaaatacagagttgaatatactcagatgtatccagggaaagataacagagccataagtgtgatccttggtgacaggtggaagaaaatgaagaatgaagagagaagaatgtacacattagaagcaaaggctttggctgaagaacagaaacgtttaaatcctgactgttggaagaggaaaagaaccaattcaggctcacaacaacattaaaccaggatgcttatgttcttaagtctatatttgcatatacattgactcttgatggaaagacttaagaagatcaaggtctcaccatttgtcctcaattcgtgtgaccataagatactgatagcattgagtcttgaaatgatttaataatatgagtgaggatttgctttctccattagagcattaagctaaaactatcaacattttaaaccaaattgccttatttttcttccaaacttcatatatgtctatcaggtaataataggcttgaaaattgatatcctgtggtgctaaagtacagtagaaagagaggagaagtgtatacatgttttattttaaattgtacgaaaggggaatttaaaaaatatgtaactgctgtttatacattggctccttactgcttattaatctgtattgtacacatgatgaaatgaagcagaagctgggagtcggcctttcctctagtaaccaccacatggctcagcatctgtgccaaacataggcgctcctagtctggtcagtgccaagaggctaccagaacatggggcaggtggctggtgttggtgtcccagcctaagagccacctgctgcagttaccatggcatgctgagttgatgcaccaggtggcagcagccatccgttattatttccaatggagacctagcccaggccaaggtaaagttagttaatagcattgggatatagtcactgtaatggtgctattaacaaacagtcaacaccattgtattttttaacttcgtgttctgtatctcctcagccatgtatcttaaatatattttgtcatcataatctttatggtgggggcagactttgcacttactgcagtgcaacacttgcactttaattttcctccaactgtctaaaattagagcaaatacattggcaatacagctgcttttgctctgagctacaatcatggcttttcatgttacttaccaagtggtgtttctggttaggaatcacagctgtaaaattgatttcagttcatcacacttcttcatgatgttgcccctaaattttgcacactatattcttgtatattatttcaaataaatggaaaaa

SEQ ID NO: 54 - DNA, 3084 bp, Homo sapiens
aagatctaaaaacggacatctccaccgtgggtggctcctttttctttttctttttttcccacccttcaggaagtggacgtttcgttatcttctgatccttgcaccttcttttggggaaacggggcccttctgcccagatcccctctcttttctcggaaaacaaactactaagtcggcatccggggtaactacagtggagagggtttccgcggagacgcgccgccggaccctcctctgcactttggggaggcgtgctccctccagaaccggcgttctccgcgcgcaaatcccggcgacgcggggtcgcggggtggccgccggggcagcctcgtctagcgcgcgccgcgcagacgcccccggagtcgccagctaccgcagccctcgccgcccagtgcccttcggcctcggggcgggcgcctgcgtcggtctccgcgaagcgggaaagcgcggcggccgccgggattcgggcgccgcggcagctgctccggctgccggccggcggccccgcgctcgcccgccccgcttccgcccgctgtcctgctgcacgaacccttccaactctcctttcctcccccacccttgagttacccctctgtctttcctgctgttgcgcgggtgctcccacagcggagcggagattacagagccgccgggatgccccaactctccggaggaggtggcggcggcgggggggacccggaactctgcgccacggacgagatgatccccttcaaggacgagggcgatcctcagaaggaaaagatcttcgccgagatcagtcatcccgaagaggaaggcgatttagctgacatcaagtcttccttggtgaacgagtctgaaatcatcccggccagcaacggacacgaggtggccagacaagcacaaacctctcaggagccctaccacgacaaggccagagaacaccccgatgacggaaagcatccagatggaggcctctacaacaagggaccctcctactcgagttattccgggtacataatgatgccaaatatgaataacgacccatacatgtcaaatggatctctttctccacccatcccgagaacatcaaataaagtgcccgtggtgcagccatcccatgcggtccatcctctcacccccctcatcacttacagtgacgagcacttttctccaggatcacacccgtcacacatcccatcagatgtcaactccaaacaaggcatgtccagacatcctccagctcctgatatccctactttttatcccttgtctccgggtggtgttggacagatcaccccacctcttggctggcaaggtcagcctgtatatcccatcacgggtggattcaggcaaccctacccatcctcactgtcagtcgacacttccatgtccaggttttcccatcatatgattcccggtcctcctggtccccacacaactggcatccctcatccagctattgtaacacctcaggtcaaacaggaacatccccacactgacagtgacctaatgcacgtgaagcctcagcatgaacagagaaaggagcaggagccaaaaagacctcacattaagaagcctctgaatgcttttatgttatacatgaaagaaatgagagcgaatgtcgttgctgagtgtactctaaaagaaagtgcagctatcaaccagattcttggcagaaggtggcatgccctctcccgtgaagagcaggctaaatattatgaattagcacggaaagaaagacagctacatatgcagctttatccaggctggtctgcaagagacaattatggtaagaaaaagaagaggaagagagagaaactacaggaatctgcatcaggtacaggtccaagaatgacagctgcctacatctgaaacatggtggaaaacgaagctcattcccaacgtgcaaagccaaggcagcgaccccaggacctcttctggagatggaagcttgttgaaaacccagactgtctccacggcctgcccagtcgacgccaaaggaacactgacatcaattttaccctgaggtcactgctagagacgctgatccataaagacaatcactgccaacccctctttcgtctactgcaagagccaagttccaaaataaagcataaaaaggttttttaaaaggaaatgtaaaagcacatgagaatgctagcaggctgtggggcagctgagcagcttttctccccccatatctgcgtgcacttcccagagcatcttgcatccaaacctgtaacctttcggcaaggacggtaacttggctgcatttgcctgtcatgcgcaactggagccagcaaccagctatccatcagcaccccagtggaggagttcatggaagagttccctctttgtttctgcttcatttttctttcttttcttttctcctaaagcttttatttaacagtgcaaaaggatcgtttttttttgcttttttaaacttgaatttttttaatttacactttttagttttaattttcttgtatattttgctagctatgagcttttaaataaaattgaaagttctggaaaagtttgaaataatgacataaaaagaagccttctttttctgagacagcttgtctggtaagtggcttctctgtgaattgcctgtaacacatagtggcttctccgcccttgtaaggtgttcagtagagctaaataaatgtaatagccaaaccccactctgttggtagcaattggcagccctatttcagtttattttttcttctgttttcttcttttctttttttaaacagtaaaccttaacagatgcgttcagcagactggtttgcagtgaattttcatttctttccttatcacccccttgttgtaaaaagcccagcacttgaattgttattactttaaatgttctgtatttgtatctgtttttattagccaattagtgggattttatgccagttgttaaaatgagcattgatgtacccattttttaaaaaagcaagcacagcctttgcccaaaactgtcatcctaacgtttgtcattccagtttgagttaatgtgctgagcatttttttaaaagaagctttgtaataaaacatttttaaaaattgtcatttaaaaaaaaaaaaaaaaaa

SEQ ID NO: 55 - DNA, 2727 bp, Homo sapiens
tttcccttttttttccgtttggcaacccacacccttccacacacgctcacccgaaaattaaacaccaagatcctctaacttgtttggattgactgatgaagacataaagctctatgttttttgaggtggagtgagtggtttttcttcatttttaaatggccaaatgacagcttgacccagtttgctttccaatcaaagggcatttattttgaatgtctctttgtggcgcaagagccaacgcaaaaatgatggcggcttacaatggcggtacatctgcagcagcagcaggtcaccaccaccaccatcaccaccaccttccacacctccctcctcctcacctgcaccaccaccaccaccctcaacaccatcttcatccggggtcggctgccgctgtacaccctgtacagcagcacacctcttcggcagctgcggcagccgcagcagcggctgcagctgcagccatgttaaaccctgggcaacaacagccatatttcccatcaccggcaccggggcaggctcctggaccagctgcagcagccccagctcaggtacaggctgccgcagctgctacagttaaggcgcaccatcatcagcactcgcatcatccacagcagcagctggatattgagccggatagacctattggatatggagcctttggtgttgtctggtcagtaacagatccaagagatggaaagagagtagcgctcaaaaagatgcccaacgtcttccagaatctggtctcttgcaaaagggtcttccgggaattgaagatgttgtgtttttttaagcatgataatgtactctctgcccttgacatactccaacctccacacattgactattttgaagaaatatatgttgtcacagaattgatgcagagtgacctacataaaattatcgtctctcctcaaccactcagctcagatcatgtcaaagtttttctttatcagattttgcgaggtttgaaatatctccattcagctggcattttacatcgagacattaagccagggaatctccttgtgaacagcaactgtgttctaaagatttgtgattttggattggccagagtggaagaattagatgaatcccgtcatatgactcaggaagttgttactcagtattatcgggctccagaaatcctgatgggcagccgtcattacagcaatgctattgacatctggtctgtgggatgtatctttgcagaactactaggacgaagaatattgtttcaggcacagagtcccattcagcagttggatttgatcacggatctgttgggcacaccatcactggaagcaatgaggacagcttgtgaaggcgctaaggcacatatactcaggggtcctcataaacagccatctcttcctgtactctataccctgtctagccaggctacacatgaagctgttcatctcctttgcaggatgttggtctttgatccatccaaaagaatatccgctaaggatgccttagcccacccctacctagatgaagggcgactacgatatcacacatgtatgtgtaaatgttgcttttccacctccactggaagagtttataccagtgactttgagcctgtcaccaatcccaaatttgatgacactttcgagaagaacctcagttctgtccgacaggttaaagaaattattcatcagttcattttggaacagcagaaaggaaacagagtgcctctctgcatcaaccctcagtctgctgcttttaagagctttattagttccactgttgctcagccatctgagatgcccccatctcctctggtgtgggagtgatggtggaagataatgtactactgaagatgtaatgtagctttccactggagtctgggatttgcaattctggaggttaatcatgcttgtactgtaattttactaatgaagttttaaattaacaaccactacttgtatgatatgaataatatttagaaatgttactagacttttaatcttgtaaagtggttgtgcttttagaagaaaaatattttacccagagttgcacatgttttatgaatttagtgcagctgttatggctcacctcagaacaaaagagaattgaaccaaatttgggagtttggggttttatgttttgtttttcttttctaaaatgaagtgagattgttcacacacacacacacacacacacacacacacaaacacaaaggacagtcatacattttgatatttgagccattcctaaagatttggggttttctaaaactaaagaatctaggaaccttgcctgcgaccaatcatggagccacgtgagctgatcgtggctgcacctggggggagggtagggaggaggggcatgccacctaatgatcaagccctataattagcttctcattagagccgtgatggtgatgtgtgctgtctaaaatccaatgttgtgggtagagagaatgagtttgtgactaggagagactaaacttttgttttccttacccagtataaatatatatatatatatttaatctatttttattagaagtttttctgctctttcttacataaaagaaccccaagcatgcatctttcatgtgtgtaaataattcatttctgggctaatttcaaaagaatcccaatattgctgtatagaaagagaactagcttgcacattttaggtctgtgaaattttgtgagacttttcctgcactggacagtaaaaaaataataaaagacaaaaacaaatttaaaaaaaaaa

SEQ ID NO: 56 - DNA, 1518 bp, Homo sapiens
gcggccgcgtcgacgagcatcatggcaaccgttacagccacaaccaaagtcccggagatccgtgatgtaacaaggattgagcgaatcggtgcccactcccacatccggggactggggctggacgatgccttggagcctcggcaggcttcgcaaggcatggtgggtcagctggcggcacggcgggcggctggcgtggtgctggagatgatccgggaagggaagattgccggtcgggcagtccttattgctggccagccgggcacggggaagacggccatcgccatgggcatggcgcaggccctgggccctgacacgccattcacagccatcgccggcagtgaaatcttctccctggagatgagcaagaccgaggcgctgacgcaggccttccggcggtccatcggcgttcgcatcaaggaggagacggagatcatcgaaggggaggtggtggagatccagattgatcgaccagcaacagggacgggctccaaggtgggcaaactgaccctcaagaccacagagatggagaccatctacgacctgggcaccaagatgattgagtccctgaccaaggacaaggtccaggccggggacgtgatcaccatcgacaaggcgacgggcaagatctccaagctgggccgctccttcacacgcgcccgcgactacgacgctatgggctcccagaccaagttcgtgcagtgcccagatggggagctccagaaacgcaaggaggtggtgcacaccgtgtccctgcacgagatcgacgtcatcaactctcgcacccagggcttcctggcgctcttctcaggtgacacaggggagatcaagtcagaagtccgtgagcagatcaatgccaaggtggctgagtggcgcgaggagggcaaggcggagatcatccctggagtgctgttcatcgacgaggtccacatgctggacatcgagagcttctccttcctcaaccgggccctggagagtgacatggcgcctgtcctgatcatggccaccaaccgtggcatcacgcgaatccggggcaccagctaccagagccctcacggcatccccatagacctgctggaccggctgcttatcgtctccaccaccccctacagcgagaaagacacgaagcagatcctccgcatccggtgcgaggaagaagatgtggagatgagtgaggacgcctacacggtgctgacccgcatcgggctggagacgtcactgcgctacgccatccagctcatcacagctgccagcttggtgtgccggaaacgcaagggtacagaagtgcaggtggatgacatcaagcgggtctactcactcttcctggacgagtcccgctccacgcagtacatgaaggagtaccaggacgccttcctcttcaacgaactcaaaggcgagaccatggacacctcctgagttggatgtcatcccccgaccccaccctgttttccaccagagttctgacactgtgactctgtataaaatggttgggaagctgcaaaaaaaaaaaaaaaaaaaaaa

SEQ ID NO: 57 - DNA, 932 bp, Homo sapiens
gggcgtgagaaaggcgacggcggcggcgcggaggagggttatctatacatttaaaaaccagccgcctgcgccgcgcctgcggagacctgggagagtccggccgcacgcgcgggacacgagcgtcccacgctccctggcgcgtacggcctgccaccactaggcctcctatccccgggctccagacgacctaggacgcgtgccctggggagttgcctggcggcgccgtgccagaagcccccttggggcgccacagttttccccgtcgcctccggttcctctgcctgcaccttcctgcggcgcgccgggacctggagcgggcgggtggatgcaggcgcgatggacggcggcacactgcccaggtccgcgccccctgcgccccccgtccctgtcggctgcgctgcccggcggagacccgcgtccccggaactgttgcgctgcagccggcggcggcgaccggccaccgcagagaccggaggcggcgcagcggccgtagcgcggcgcaatgagcgcgagcgcaaccgcgtgaagctggtgaacttgggcttccaggcgctgcggcagcacgtgccgcacggcggcgccagcaagaagctgagcaaggtggagacgctgcgctcagccgtggagtacatccgcgcgctgcagcgcctgctggccgagcacgacgccgtgcgcaacgcgctggcgggagggctgaggccgcaggccgtgcggccgtctgcgccccgcgggccgccagggaccaccccggtcgccgcctcgccctcccgcgcttcttcgtccccgggccgcgggggcagctcggagcccggctccccgcgttccgcctactcgtcggacgacagcggctgcgaaggcgcgctgagtcctgcggagcgcgagctactcgacttctccagctggttagggggctactgagcgccctcgaccta

SEQ ID NO: 58 - DNA, 5182 bp, Homo sapiens
tctcttcccctctccccttctcttccccgaggctatgtccacccggtgcggcgaggcgggcagagccagaggcacgcagccgcacaggggctacagagcccagaatcagccctacaagatgcacttaggacccccgcggctggaagaatgagcttgtccttcctcctcctcctcttcttcagccacctgatcctcagcgcctgggctcacggggagaagcgtctcgcccccaaagggcaacccggacccgctgccactgataggaaccctagaggctccagcagcagacagagcagcagtagcgctatgtcttcctcttctgcctcctcctcccccgcagcttctctgggcagccaaggaagtggcttggagcagagcagtttccagtggagcccctcggggcgccggaccggcagcctctactgcagagtgggcatcggtttccatctgcagatctacccggatggcaaagtcaatggatcccacgaagccaatatgttaagccaagttcacagatgactgcaagttcagggagcgttttcaagaaaatagctataatacctatgcctcagcaatacatagaactgaaaaaacagggcgggagtggtatgtggccctgaataaaagaggaaaagccaaacgagggtgcagcccccgggttaaaccccagcatatctctacccattttctgccaagattcaagcagtcggagcagccagaactttctttcacggttactgttcctgaaaagaaaaagccacctagccctatcaagccaaagattcccctttctgcacctcggaaaaataccaactcagtgaaatacagactcaagtttcgctttggataatattcctcttggccttgtgagaaaccattctttcccctcaggagtttctataggtgtcttcagagttctgaagaaaaattactggacacagcttcagctatacttacactgtattgaagtcacgtcatttgtttcaatgtgactgaaacaaaatgttttttgataggaaggaaactggaattctttgtactaatacagggagcacactccttcagttcagcaagacataaagccttttgctttatgcttgagggatatttagaactttgtattttcggaaagttaaataacagggactacgtatttttctgacttttacagattaacctgaaagaacatacatgatacatttttatttttggtttccaaagaatattttgatgcagataaaatattttgttaacttttgtttttttttgtttgttttcttaaaagtacctctgcattgagcatattttcttacttttattattttaattaatatgacataagcaatcattttatgctgtttatgaattataaatgtgtttatagctcatttgtaatatggaaatcttttacatttttcctattcactgcacttttttattgtttttatttctagccatacctcagataatatgtttagttttacattttaaaatgtttaaattctctttcacagcaccaaaggctcagcttggatttgtgtgtatgtgtatgtcaattcatgacattatgtggaatcctaaacctttggtggctgggatatgatgggttagaagcaaggagaaaatataaggactttttgatggaattaaatgtgggaggtaaggaaaaggatttagaggtaaaagtacactaagtttgcaacatttattgagatctaagtctgtcttgccttcatttctctttttatctcccccttgccctcattcttgaacagctggaggaatacattttattctgtccatgaagcatacactatgaaattcaagtgcttaaaaatacttctatgactctctgctatcccactgtatagatccacagggagcaaacacttagaaatgatagagaactgaaggagatcaatggtttaacagttatccatgccaagtcccattgtcagaaatattcttattactcagtcaaacactctttgagcttcccttcctaaaggtaaccaatccagtgaatagatgtgcccttttataaggaaacttctgatgtttattaaaaaaactggccttttgatagaggtaacttaatttgggaatttgttgtgttgaaatggcatttaatttcaacctaaatactgactgctggacataaatcacagaaaatttaacttaagaaaatttacaaaatttattctcaggtaatcattttaataaagttctgcaaaatacacgtttatcttacattcagaaatgtggcaaaaaaggcatagctaaaggctaaacatatggctttagtagtaacaaaagggttcatagaaacttcatggtttgcatttaaacatgtttaaagtgtacttataaactatttttttcttaaagcaaactatgatttattttggtgcacaaatacaaagtggaaacttaccaaaattgaactagctaccatataagcagattgctttaatttgatgggaaaatagtacacacatatatataacaaataatatattaaaaaacccatccatcaactaaaacattatatgtatacatcagtatagtgttttattataaagccaattatctgattaagcattctttccactgaatgcataatgtttaaatagcataaaatgaaatgctacaaaaattgaactaatttatactttaaagtatttctgggttaaatgaaacaatgaaattttttagtatgttcaactctcatccaaatggcatatgaccctgtttacacagcctaaagctaaaaatattactctagtttattctaatctattgttaagtattgtgcactgtataccaagttcttagggcacatgaaaaattttagctgccaaacaggaactagtaaacatatgttcctaataagtgaagggaaagataataatgatggtcaacaataagccacgtcaatgcataagttgtataggctaaatgttgcttgtaggctacattaaactcaaatgtaatagtttatcttatactcctggtttgatttgattagcatattaacgtgaaagtaggatagctactaaatatatattatgcaagtcaggaatcattaatttcaaaatttaaagccatgctaaaattaaaaagaaaatattaaattacacaattacacttgtctttactggccatacaaaatgattttttttttttttttgagacagagtcttgctctgtcaccaggctggagtgcagtggcatgatctcggctcactgcaacctccaactccctggtttaagggattctcctgcctcagcctcccaagtagctgggattacagactcatgccaccacgccagctaatttttgtatttttagtagagacggggtttcaccatgttggtcaggatggtctcaatcctggcctcttgatagtcctgacctcatgatctgcccacctcggcctccccaaagtgctgggattacaggtacaatgatgtataattaatgcttagtgaagcataaagttacctacatcaattaattaaatgaacttatgtacagaaaacatgtataaatataagtctatactaatgcttacaactttctaagagggttcttgcttatgtagctttttattattttaagtaactagaaccaccaaatatcaaataaaattatttggttatggttatgttcatctaaacacaacaataacttttatattaatatttaggagtctattttgtctataggtgacaaacatctccagactaacatgtcagttttatcaattatattatgtttaattatttaagatttctttatgtggaacatctatagagataaatagaaattttcaataagatgtagtaacactgtgatttatctttcaagagtctctcttcacttccttctaaagagactaatttgagagtacaggtgcatattaattttcttggttctttcagctgaattatattggtccagaagttcaaaatcatgtgacaataataagggatactgacagaagttatttccaagtttgtgtatatattataaaaattacatatataaaactaaggcttttatttctgttatttttaagcttttatttcttgtagctaaaaataaaacatcataaatctggtaggtaaatttcttattaaatcaatcttgaaatagaaaatgtaataactttcttaccattaacattttttacccttccatagaagggagggaataaatcatgacttatcccattttcaataacaaaacgaaactatggcactaaccaaaaacttgcattctggcataatttttacagttgcagagaattgtttctgggctcattaaaaaaagtagtattgcagacattgctgcaatgggaagcagacaataacttcttaaaggaattctacacctcctttaagatttacttaattgctacatctaaattctgataatttaaaatccattttaggtgataaaattttttaaaagttttgaaggaaacctctggataaatggacaaggcctaatttttttttgtagtcaatccaactgtactggccaatttttgaaataagattatatgattaggtattagcagagacaaagagttacctcctccatcttactctgccctatttgaaagtctcaggggagaaaagggaacaagatgctgatccaacctgagtggagtcaggtgaggcatctttacatctaagaatttttttttaaattttattattattatacttcaagttctagggtacatgtccacaatgcacatgtctgtcacacatgcacacatgtgccatgctggtgtgctgcacccaccaacctgtcatccagcattaggtatatctcctaatgctatccctcccctctccacccaccccacagcaggccccggtatgtgatgttccccttcgtgtgtccatgtgttcttattgttcaattcccacctatgagtgagaatatgtggtgtttggtttttggtccttgcaatagtttgctgagaatgatggtttccagcttcatccatgtccctacaaagaacatgaactcatcatttttgatggctgcatagtattccatggtgtatatgtgccacattttcttaatccagtctatcattgttggacatttgggttggttccagtctttgctattgtgaatagtgctgcaataaacatatgtgtg

SEQ ID NO: 59 - DNA, 1873 bp, Homo sapiens
tgcgtgcccccaggcccagcgcgcctccgggcgagtccccaggagcgcggcccaatggatcgctccgggcccgccccctcgcgcgctgattggccgccgccccgctggcctcgccttattagcaagttctctggggagccgcggtagggcccggagccggcgagtgctcccgggaactctgcctgcgcggcggcagcgaccggaggccaggcccagcacgccggagctggcctgctggggaggggcgggaggcgcgcgcgggagggtccgcccggccagggccccgggcgctcgcagaggccggccgcgctcccagcccgcccggagcccatgcccggcggctggccagtgctgcggcagaagggggggcccggctctgcatggccccggctgctgacatgacttctttgccactcggtgtcaaagtggaggactccgccttcggcaagccggcggggggaggcgcgggccaggcccccagcgccgccgcggccacggcagccgccatgggcgcggacgaggagggggccaagcccaaagtgtccccttcgctcctgcccttcagcgtggaggcgctcatggccgaccacaggaagccgggggccaaggagagcgccctggcgccctccgagggcgtgcaggcggcgggtggctcggcgcagccactgggcgtcccgccggggtcgctgggagccccggacgcgccctcttcgccgcggccgctcggccatttctcggtggggggactcctcaagctgccagaagatgcgctcgtcaaagccgagagccccgagaagcccgagaggaccccgtggatgcagagcccccgcttctccccgccgccggccaggcggctgagccccccagcctgcaccctccgcaaacacaagacgaaccgtaagccgcggacgcccttcaccaccgcgcagctgctggcgctggagcgcaagttccgccagaagcagtacctgtccatcgccgagcgcgcggagttctccagctcgctcagcctcactgagacgcaggtgaagatatggttccagaaccgccgcgccaaggcaaagagactacaagaggcagagctggagaagctgaagatggccgccaagcccatgctgccaccggctgccttcggcctctccttccctctcggcggccccgcagctgtagcggccgcggcgggtgcctcgctctacggtgcctctggccccttccagcgcgccgcgctgcctgtggcgcccgtgggactctacacggcccatgtgggctacagcatgtaccacctgacatagagggtcccaggtcgcccacctgtgggccagccgattcctccagccctggtgctgtacccccggacgtgctcccctgctcggcaccgccagccgccttccctttaaccctcacactgctccagtttcacctctttgctccctgagttcactctccgaagtctgatccctgccaaaaagtggctggaagagtcccttagtactcttctagcatttagagatctaccctctcgagttaaaagatggggaaactgagggcagagaggttaacagatttatctaaggtccccagcagaattgacagtgtgaacagagctagaggccatgtctcctgcatagtttttccctgtcctgacaccaggcaagaaagcgcagagaaatcggtgtctgacgattttggaatgagaacaatctcaaaaaaaaaaaaaaaaaaaaaaaaaagaaaagagaaaaaaaagactagccaccaggaagatgaatcctagcttcttccattggaaaatttaaagacaagttcaacaacaaaacatttgctctggggggcagggaaaacacagatgtgttgcaaaggtaggttgaaggga

SEQ ID NO: 60 - DNA, 714 bp, Homo sapiens
atgccagcccgccttgagacctgcatctccgacctcgactgcgccagcagcagcggcagtgacctatccggcttcctcaccgacgaggaagactgtgccagactccaacaggcagcctccgcttcggggccgcccgcgccggcccgcaggagcgcgcccaatatctcccgggcgtctgaggttccaggggcacaggacgacgagcaggagaggcggcggcgccgcggccggacgcgggtccgctccgaggcgctgctgcactcgctgcgcaggagccggcgcgtcaaggccaacgatcgcgagcgcaaccgcatgcacaacttgaacgcggccctggacgcactgcgcagcgtgctgccctcgttccccgacgacaccaagctcaccaaaatcgagacgctgcgcttcgcctacaactacatctgggctctggccgagacactgcgcctggcggatcaagggctgcccggaggcggtgcccgggagcgcctcctgccgccgcagtgcgtcccctgcctgcccggtcccccaagccccgccagcgacgcggagtcctggggctcaggtgccgccgccgcctccccgctctctgaccccagtagcccagccgcctccgaagacttcacctaccgccccggcgaccctgttttctccttcccaagcctgcccaaagacttgctccacacaacgccctgtttcattccttaccactag

SEQ ID NO: 61 - DNA, 754 bp, Homo sapiens
ggcctcccccgccttggcggccctgaccccgctgtcatccagcgccgacgaagaagaggaggaggagccgggcgcgtcaggcggggcgcgtcggcagcgcggggctgaggccgggcagggggcgcggggcggcgtggctgcgggtgcggagggctgccggcccgcacggctgctgggtctggtacacgattgcaaacggcgcccttcccgggcgcgggccgtctcccgaggcgccaagacggccgagacggtgcagcgcatcaagaagacccgtagactgaaggccaacaaccgcgagcgaaaccgcatgcacaacctcaacgcggcactggacgcgctgcgcgaggtgctccccacgttccccgaggacgccaagctcaccaagatcgagaccctgcgcttcgcccacaactacatctgggcactcaccgagaccctgcgcctggcggatcactgcgggggcggcggcgggggcctgccgggggcgctcttctccgaggcagtgttgctgagcccgggaggagccagcgccgccctgagcagcagcggagacagcccctcgcccgcctccacgtggagttgcaccaacagccccgcgccgtcctcctccgtgtcctccaattccacctccccctacagctgcactttatcgcccgccagcccggccgggtcagacatggactattggcagcccccacctcccgacaagcaccgctatgcacctcacctccccatagccagggattgtatctag

SEQ ID NO: 62 - DNA, 1167 bp, Homo sapiens
cctcggaccccattctctcttcttttctcctttggggctggggcaactcccaggcgggggcgcctgcagctcagctgaacttggcgaccagaagcccgctgagctccccacggccctcgctgctcatcgctctctattcttttgcgccggtagaaaggatgacgcctcaaccctcgggtgcgcccactgtccaagtgacccgtgagacggagcggtccttccccagagcctcggaagacgaagtgacctgccccacgtccgccccgcccagccccactcgcacacgggggaactgcgcagaggcggaagagggaggctgccgaggggccccgaggaagctccgggcacggcgcgggggacgcagccggcctaagagcgagttggcactgagcaagcagcgacggagtcggcgaaagaaggccaacgaccgcgagcgcaatcgaatgcacaacctcaactcggcactggacgccctgcgcggtgtcctgcccaccttcccagacgacgcgaagctcaccaagatcgagacgctgcgcttcgcccacaactacatctgggcgctgactcaaacgctgcgcatagcggaccacagcttgtacgcgctggagccgccggcgccgcactgcggggagctgggcagcccaggcggttcccccggggactgggggtccctctactccccagtctcccaggctggcagcctgagtcccgccgcgtcgctggaggagcgacccgggctgctgggggccaccttttccgcctgcttgagcccaggcagtctggctttctcagattttctgtgaaaggacctgtctgtcgctgggctgtgggtgctaagggtaagggagagggagggagccgggagccgtagagggtggccgacggcggcggccctcaaaagcacttgttccttctgcttctccctggctgacccctggccggcccaggcctccacgggggcggcaggctgggttcattccccggccctccgagccgcgccaacgcacgcaacccttgctgctgcccgcgcgaagtgggcattgcaaagtgcgctcattttaggcctcctctctgccaccaccccataatctcattcaaagaatactagaatggtagcactacccggccggagccgcccaccgtcttgggtcgccctaccctcactca

SEQ ID NO: 63 - DNA, 3160 bp, Homo sapiens
cctcccctcgcccggcgcggtcccgtccgcctctcgctcgcctcccgcctcccctcggtcttccgaggcgcccgggctcccggcgcggcggcggagggggcgggcaggccggcgggcggtgatgtggcaggactctttatgcgctgcggcaggatacgcgctcggcgctgggacgcgactgcgctcagttctctcctctcggaagctgcagccatgatggaagtttgagagttgagccgctgtgaggcgaggccgggctcaggcgagggagatgagagacggcggcggccgcggcccggagcccctctcagcgcctgtgagcagccgcgggggcagcgccctcggggagccggccggcctgcggcggcggcagcggcggcgtttctcgcctcctcttcgtcttttctaaccgtgcagcctcttcctcggcttctcctgaaagggaaggtggaagccgtgggctcgggcgggagccggctgaggcgcggcggcggcggcggcggcacctcccgctcctggagcgggggggagaagcggcggcggcggcggccgcggcggctgcagctccagggagggggtctgagtcgcctgtcaccatttccagggctgggaacgccggagagttggtctctccccttctactgcctccaacacggcggcggcggcggcggcacatccagggacccgggccggttttaaacctcccgtccgccgccgccgcaccccccgtggcccgggctccggaggccgccggcggaggcagccgttcggaggattattcgtcttctccccattccgctgccgccgctgccaggcctctggctgctgaggagaagcaggcccagtcgctgcaaccatccagcagccgccgcagcagccattacccggctgcggtccagagccaagcggcggcagagcgaggggcatcagctaccgccaagtccagagccatttccatcctgcagaagaagccccgccaccagcagcttctgccatctctctcctcctttttcttcagccacaggctcccagacatgacagccatcatcaaagagatcgttagcagaaacaaaaggagatatcaagaggatggattcgacttagacttgacctatatttatccaaacattattgctatgggatttcctgcagaaagacttgaaggcgtatacaggaacaatattgatgatgtagtaaggtttttggattcaaagcataaaaaccattacaagatatacaatctttgtgctgaaagacattatgacaccgccaaatttaattgcagagttgcacaatatccttttgaagaccataacccaccacagctagaacttatcaaacccttttgtgaagatcttgaccaatggctaagtgaagatgacaatcatgttgcagcaattcactgtaaagctggaaagggacgaactggtgtaatgatatgtgcatatttattacatcggggcaaatttttaaaggcacaagaggccctagatttctatggggaagtaaggaccagagacaaaaagggagtaactattcccagtcagaggcgctatgtgtattattatagctacctgttaaagaatcatctggattatagaccagtggcactgttgtttcacaagatgatgtttgaaactattccaatgttcagtggcggaacttgcaatcctcagtttgtggtctgccagctaaaggtgaagatatattcctccaattcaggacccacacgacgggaagacaagttcatgtactttgagttccctcagccgttacctgtgtgtggtgatatcaaagtagagttcttccacaaacagaacaagatgctaaaaaaggacaaaatgtttcacttttgggtaaatacattcttcataccaggaccagaggaaacctcagaaaaagtagaaaatggaagtctatgtgatcaagaaatcgatagcatttgcagtatagagcgtgcagataatgacaaggaatatctagtacttactttaacaaaaaatgatcttgacaaagcaaataaagacaaagccaaccgatacttttctccaaattttaaggtgaagctgtacttcacaaaaacagtagaggagccgtcaaatccagaggctagcagttcaacttctgtaacaccagatgttagtgacaatgaacctgatcattatagatattctgacaccactgactctgatccagagaatgaaccttttgatgaagatcagcatacacaaattacaaaagtctgaatttttttttatcaagagggataaaacaccatgaaaataaacttgaataaactgaaaatggacctttttttttttaatggcaataggacattgtgtcagattaccagttataggaacaattctcttttcctgaccaatcttgttttaccctatacatccacagggttttgacacttgttgtccagttgaaaaaaggttgtgtagctgtgtcatgtatatacctttttgtgtcaaaaggacatttaaaattcaattaggattaataaagatggcactttcccgttttattccagttttataaaaagtggagacagactgatgtgtatacgtaggaattttttccttttgtgttctgtcaccaactgaagtggctaaagagctttgtgatatactggttcacatcctacccctttgcacttgtggcaacagataagtttgcagttggctaagagaggtttccgaaaggttttgctaccattctaatgcatgtattcgggttagggcaatggaggggaatgctcagaaaggaaataattttatgctggactctggaccatataccatctccagctatttacacacacctttctttagcatgctacagttattaatctggacattcgaggaattggccgctgtcactgcttgttgtttgcgcatttttttttaaagcatattggtgctagaaaaggcagctaaaggaagtgaatctgtattggggtacaggaatgaaccttctgcaacatcttaagatccacaaatgaagggatataaaaataatgtcataggtaagaaacacagcaacaatgacttaaccatataaatgtggaggctatcaacaaagaatgggcttgaaacattataaaaattgacaatgatttattaaatatgttttctcaattgtaaaaaaaaaa

SEQ ID NO: 64 - DNA, 1528 bp, Homo sapiens
gagggaagtgtcgactgcgccggcgggaacagacatgcctgcggccaagaagcagaagctgagcagtgacgagaacagcaatccagaactctctggagacgagaatgatgacgctgtcagtatagaaagtggtacaaacactgaacgccctgatacacctacaaacacgccaaatgcacctggaaggaaaagttggggaaagggaaaatggaagtcaaagaaatgcaaatattctttcaaatgtgtaaatagtctcaaggaagatcataaccaaccattgtttggagttcagtttaactggcacagtaaagaaggagatccattagtgtttgcaactgtaggaagcaacagagttaccttgtatgaatgtcattcacaaggagaaatccggttgttgcaatcttacgtggatgctgatgctgatgaaaacttttacacttgtgcatggacctatgatagcaatacgagccatcctctgctggctgtagctggatctagaggcataattaggataataaatcctataacaatgcagtgtataaagcactatgttggccatggaaatgctatcaatgagctgaaattccatccaagagatccaaatcttctcctgtcagtaagtaaagatcatgctttacgattatggaatatccagacggacactctggtggcaatatttggaggcgtagaagggcacagagatgaagttctaagtgctgattatgatcttttgggtgaaaaaataatgtcctgtggtatggatcattctcttaaactttggaggatcaattcaaagagaatgatgaatgcaattaaggaatcttatgattataatccaaataaaactaacaggccatttatttctcagaaaatccattttcctgatttttctaccagagacatacataggaattatgttgattgtgtgcgatggttaggcgatttgatactttctaagtcttgtgaaaatgccattgtgtgctggaaacctggcaagatggaagatgatatagataaaattaaacccagtgaatctaatgtgactattcttgggcgatttgattacagccagtgtgacatttggtacatgaggttttctatggatttctggcaaaagatgcttgcattgggcaatcaagttggcaaactttatgtttgggatttagaagtagaagatcctcataaagccaaatgtacaacactgactcatcataaatgtggtgctgctattcgacaaaccagttttagcagggatagcagcattcttatagctgtttgtgatgatgccagtatttggcgctgggatcgacttcgataaaatacttttgcctaatcaaaattaaaatgtgtttgttgtctgtgtaaaataaaattaatgtatcttgctagtaagggcacgtagagcatttagagttgtctttcagcattcaatcaggctgagctgaatgtagtgatgtttacattgtttacattctttgtactgtcttcctgctcagactctactgcttttaataaaaatttatttttg

SEQ ID NO: 65 - DNA, 2587 bp, Homo sapiens
  misc_feature (1751)..(1751): unknown nucleotide
agggcgaacgggcgagtggcagcgaggcggggcgggctgaggccagcgcggaagtctcgcgaggccgggcccgagcagagtgtggcggcggcggcgagatctgggctcgggttgaggagttggtatttgtgtggaaggaggcggaggcgcaggaggaggaagggggaagcggagcgcggcccggacgggaggaggcgcggccagggcgggcggttgcggcgaggcgaggcgaggagggggagccgagacgcagcggccgaggcgggggcgcgggcgcaccgaggcgagggaggcggggaagccccgccgccgccgcggcgcccgccccttcccccgccgcccgcgccctctccccccgccgcgctcgccgccttcctccctcgcttccttccccacggccggccgcctcctcgcccgcccgcccgcagccgaggagccgaggccgccggggccgtggcggcggagccctcagccatggcctcgggcgacaccctctacatcgccacggacggctcggagatgccggccgagatcgtggagctgcacgagatcgaggtggagaccatcccggtggagaccatcgagaccacagtggtgggcgaggaggaggaggaggacgacgacgacgaggacggcggcggtggcgaccacggcggcgggggcggccacgggcacgccggccaccaccaccaccaccatcaccaccaccaccacccgcccatgatcgctctgcagccgctggtcaccgacgacccgacccaggtgcaccaccaccaggaggtgatcctggtgcagacgcgcgaggaggtggtgggcggcgacgactcggacgggctgcgcgccgaggacggcttcgaggatcagattctcatcccggtgcccgcgccggccggcggcgacgacgactacattgaacaaacgctggtcaccgtggcggcggccggcaagagcggcggcggcggctcgtcgtcgtcgggaggcggccgcgtcaagaagggcggcggcaagaagagcggcaagaagagttacctcagcggcggggccggcgcggcgggcggcggcggcgccgacccgggcaacaagaagtgggagcagaagcaggtgcagatcaagaccctggagggcgagttctcggtcaccatgtggtcctcagatgaaaaaaaagatattgaccatgagacagtggttgaagaacagatcattggagagaactcacctcctgattattcagaatatatgacaggaaagaaacttcctcctggaggaatacctggcattgacctctcagatcccaaacaactggcagaatttgctagaatgaagccaagaaaaattaaagaagatgatgctccaagaacaatagcttgccctcataaaggctgcacaaagatgttcagggataactcggccatgagaaaacatctgcacacccacggtcccagagtccacgtctgtgcagaatgtggcaaagcttttgttgagagttcaaaactaaaacgacaccaactggttcatactggagagaagccctttcagtgcacgttcgaaggctgtgggaaacgcttttcactggacttcaatttgcgcacacatgtgcgaatccataccggagacaggccctatgtgtgccccttcgatggttgtaataagaagtttgctcagtcaactaacctgaaatctcacatcttaacacatgctaaggccaaaaacaaccagtgaaaagaagagagaagacccttctcgaccacggnaagcatcttccagaagtgtgattgggaataaatatgcctctcctttgtatattatttctaggaagaattttaaaaatgaatcctacacacctaagggacatgttttgataaagtagtaaaaaaaaaaaaaaaaaaactttactaagatgacattgctaagatgctctatcttgctctgtaatctcgtttcaaaaacacagtgtttttgtaaagtgtggtcccaacaggaggacaattcatgaacttcgcatcaaaagacaattctttatacaacagtgctaaaaatgggacttcttttcacattcttataaatatgaagctcacctgttgcttacaatttttttaattttgtattttccaagtgtgcatattgtacacttttttggggatatgcttagtaatgctacgtgtgatttttctggaggttgataactttgcttgcagtagattttctttaaaagaatgggcagttacatgcatacttcaaaagtattttcctgtaaaaaaaaaaaaagttatataggttttgtttgctatcttaattttggttgtattctttgatgttaacacattttgtataattgtatcgtatagctgtattgaatcatgtagtatcaaatattagatgtgatttaatagtgttaatcaatttaaacccattttagtcactttttttttccaaaaaaatactgccagatgctgatgttcagtgtaatttctttgcctgttcagttacagaaagtggtgctcagttgtagaatgtattgtaccttttaacacctgatgtgtacatcccatgtaacagaaagggcaacaataaaatagcaatcctaaag

SEQ ID NO: 66 - DNA, 356 bp, Homo sapiens
ctgccagagatgagtgcaggtgaggagagtagcagctcggactccgaccccgactccgcctcgccgcccagcactggacagaacccatcagtgtccgttcagaccagccaggactggaagcccacccgcagcctcatcgagcacgtatttgtcaccgacgtcactgccaacctcatcaccgtcacagtgaaggagtctcccaccagcgtcggcttcttcaacctgaggcattactgaagccccggcgccaccagctgcgcgtcttactccccttccctgcctatggtgtcgcttggctaagtgactcccagcccaagccccctcaagagtctgatcgtcgggggaggaggagtggg

SEQ ID NO: 67 - DNA, 1260 bp, Homo sapiens
attatgggctgtgggtgccgctgagcaagatggagctgtctgcagtgggcgagcgggtcttcgcggccgaatccatcatcaaacggcggatccgaaagggacgcatcgagtacctggtgaaatggaaggggtgggcgatcaagtacagcacttgggagcccgaggagaacatcctggactcgcggctcattgcagccttcgaacaaaaggagagggagcgtgagctgtatgggcccaagaagaggggacccaaacccaaaactttcctcctgaaggcgcgggcccaggccgaggccctccgcatcagtgatgtgcatttctctgtcaagccgagcgccagtgcctcctcgcccaagctgcactccagcgcagccgtgcaccggctcaagaaggacatccgccgctgccaccgtatgtcccgccgtcccctgccccgcccggacccgcaggggggcagccccggactgcgcccgcccatttcgcccttctcggagacggtgcgcatcatcaaccgcaaggtgaagccgcgggagcccaagcggaaccgcatcatcctgaacctgaaggtgatcgacaagggcgctggcggcgggggcgccgggcagggggccggggcgctggcccgccccaaagtcccctcgcggaaccgcgttataggcaagagcaagaagttcagcgagagcgtcctgcgtacacagatccgccacatgaagttcggcgcctttgcgctgtacaagcctccgcccgcccccctggtagccccgtcccccggcaaggctgaggcctcagccccgggccctgggctacttctggccgcccccgccgccccctacgacgcccgcagctctggctcctccggctgcccctcgcctacaccacagtcctctgaccccgacgacacgccccccaagctcctccccgagaccgtgagcccatccgcccccagctggcgcgagccggaggtgctcgacctgtccctccctcccgagtcggcagccaccagcaagcgggcaccgcctgaggtcacagctgctgccggcccggcacctcccacggcccctgagcccgccggtgcctcctccgagcccgaggctggggactggcgccccgagatgtcaccctgctccaatgtggtcgtcaccgatgtcaccagcaacctcctgacggtcacaatcaaggaattctgcaaccctgaggatttcgagaaggtggctgctggggtagcaggcgccgctgggggcggtggcagcattggggcga

SEQ ID NO: 68 - DNA, 1647 bp, Homo sapiens
gaattctgtgataagttattcaacttatgaaattcaagttacatgtgaattctgccaggcaatacaaggacctgtggaatatgagtgatgacaaaccctttctatgtactgcgcctggatgtggccagcgttttaccaacgaggatcatttggctgtccataaacataaacatgagatgacactgaaatttggtccagcacgtaatgacagtgtcattgtggctgatcagaccccaacaccaacaagattcttgaaaaactgtgaagaagtgggtttgtttaatgagttggcgagtccatttgagaatgaattcaagaaagcttcagaagatgacattaaaaaaatgcctctagatttatcccctcttgcaacacctatcataagaagcaaaattgaggagccttctgttgtagaaacaactcaccaggatagtcctttacctcacccagagtctactaccagtgatgagaaggaagtaccattggcacaaactgcacagcccacatcagctattgttcgtccagcatcattacaggttcccaatgtgctgcttacaagttctgactcaagtgtaattattcagcaggcagtaccttcaccaacctcaagtactgtaatcacccaggcaccatcctctaacaggccaattgtccctgtaccaggcccatttcctcttctgttacatcttcctagtggacaaaccatgcctgttgctattcctgcatcaattacaagttctaatgtgcatgttccagctgcagtcccactcgttcgaccagtcaccatggtgcctagtgttccaggaatcccaggtccttcctctccccaaccagtacagtcagaagcaaaaatgagattaaaagctgctttgacccagcaacatcctccagttaccaatggtgatactgtcaaaggtcatggtagcggattggttaggactcagtcagaggaatctcgaccgcagtcattacaacagccagccacatccactacagaaactccggcttctccagctcacacaactccacagacccaaagtacaagtggtcgtcggagaagagcagctaacgaagatcctgatgaaaaaaggagaaagtttttagagcgaaatagagcagcagcttcaagatgccgacaaaaaaggaaagtctgggttcagtctttagagaagaaagctgaagacttgagttcattaaatggtcagctgcagagtgaagtcaccctgctgagaaatgaagtggcacagctgaaacagcttcttctggctcataaagattgccctgtaaccgccatgcagaagaaatctggctatcatactgctgataaagatgatagttcagaagacatttcagtgccgagtagtccacatacggaagctatacagcatagttcggtcagcacatccaatggagtcagttcaacctccaaggcagaagctgtagccacttcagtcctcacccagatggcggaccagagtacagagcctgctctttcacagatcgttatggctccttcctcccagtcacagccctcaggaagttgattaaaaacctgcagtacaacagtttagatactcattagtgacttcaaagggaaatcaaggaaagaccagtttccatttatgcgaaatctgtggttgtaaattt

SEQ ID NO: 69 - DNA, 1542 bp, Homo sapiens
ctgacgtcagcgggagagtattatggtctgtcgtgcgctggctgctgcttttctgctcctggaagcggcctaggggggaagcggcgagtcaacatggagctttcagcggtgggggagcgggtgttcgcggccgaagccctcctgaagcggcgcatacggaaaggacgcatggaatacctcgtgaaatggaagggatggtcgcagaagtacagcacatgggaaccggaggaaaacatcctggatgctcgcttgctcgcagcctttgaggaaagggaaagagagatggagctctatggccccaaaaagcgtggacccaagcccaaaaccttcctcctcaaagcgcaggccaaggcaaaggccaaaacttacgagtttcgaagtgactcagccaggggcatccggatcccctaccctggccgctcgccccaggacctggcctccacttcccgggcccgggagggccttcgaaacatgggtttgtccccgccagcgagcagcaccagcaccagcagcacctgccgcgcagaggcccctcgggaccgggaccgagaccgggatagggaccgggagcgggatcgagaaagggagagggagcgagagagggagcgggaacgtgagagggaacgagagcggggtaccagcagagtggatgacaagcccagctcaccgggggacagctcgaagaagcgaggccccaagccccggaaggagctcccggacccctcacagaggcccttaggcgaacccagcgccggcctcggagagtacctcaagggcaggaagctggacgacaccccttccggggcaggaaagtttccagccggccacagtgtgatccagctggcccgaagacaggactcggacctggtgcagtgtggtgtgaccagccctagctcagctgaggccacgggcaaactggctgtggacaccttcccggccagggtgataaagcacagggctgccttcctggaggccaaaggccagggtgccctagatcccaatggcacccgggtccgacatggctcaggcccccccagctctggggggggcctgtaccgggacatgggggcccaggggggaaggccctccctcatcgccaggatccctgtggccagaatcctgggggacccggaggaagagtcctggagcccctccctgactaacctggagaaggtggtggtcacggacgtgacctcaaactttttgaccgtcaccattaaggaaagtaacacggaccaaggcttttttaaagagaaaagatgaatgctgggtgggtgtgtccaggaggagagcaggggagagagtgagcgtgagcttggcatagtgatttttatttctgggtgggatgtggccttttggctggtcccgtccctgatgtcacccccaccccaccagcccctttcatccctccttcctccccctcagtttttgttggaaagattatctctagagttatattttctattagatgtaaatatgttatttaagaaaaatatctaaatatatatatttcaactcgaaaaaaaaaaaaaaaaactcgag

SEQ ID NO: 70 - DNA, 2260 bp, Homo sapiens
ctcctcctgccgctgccgctgctttggctgctgcgtcatacgccccagagccgccgggacggaggggctgggcctggggaccccccggcctccgcctgcacgcccccccacgcccggacgtgccctctccgcgcgggggactcgcctaggtctcctacgtctgcccctgcccggctcccggcggccccagctgtcaccggcccccccaggatgcaatggcgcagcccccccggctgagccgctctggtgcctcctcactttgggacccagcttctcctgctcccacctctggccccaggcctcggctttgggagggtcaagatgtgctggccagatggactgatgggctgctatacttgggtaccatcaaaaaggtggacagtgctagggaggtgtgtctggtccagtttgaggatgattcgcagtttctggttctatggaaagacattagccctgctgccctccctggagaggaactcctctgttgtgtctgtcgctctgagactgtggtccctgggaaccggctggtcagctgtgagaagtgtcgccatgcttatcaccaggactgccatgttcccagggctccagcccctggagagggagagggcacatcctgggtatgccgccagtgtgtctttgcgatcgccaccaagaggggaggtgccctgaagaagggcccctatgcccgggccatgctgggtatgaagctttctctgccatatggactgaaggggctggactgggatgctggacatctgagcaaccgacagcagagttactgttactgtggtggccctggggagtggaacctgaaaatgctgcagtgccggagctgcctgcagtggttccatgaggcctgcacccagtgtctgagcaagcccctcctctatggggacaggttctatgaatttgaatgctgtgtgtgtcgcgggggccctgagaaagtccggagactacagcttcgctgggtggatgtggcccatcttgtcctgtatcacctcagtgtttgctgtaagaagaaatactttgattttgatcgtgagatcctccccttcacttctgagaattgggacagtttgctcctgggggagctttcagacacccccaaaggagaacgttcttccaagctcctctctgctcttaacagccacaaggaccgtttcatttcagggagagagattaagaagaggaaatgtttgtttggtctccatgctcggatgcctccccctgtggagccccctactggagatggagcactcaccagcttcccttcagggcagggccctgggggaggggtctcacgtcccctggggaagcgccggaggccggagccagagcccctgaggaggaggcagaaggggaaagtggaggagctggggccaccctcagcagtgcgcaatcagcccgagccccaggagcagagggagcgggctcatctgcagagggcactgcaggcctcagtgtctccaccatcccccagccctaaccagagttaccagggcagcagcggctacaacttccggcccacagatgcccgctgcctgcccagcagccccatccggatgtttgcttccttccacccttctgccagcaccgcagggacctctggggacagtggacccccagacaggtcacccctggaacttcacattggtttccccacagacatccctaaaagtgccccccactcgatgactgcctcatcttcctcagtttcatccccatccccaggtcttcctagacgctcagcacccccttctcccctgtgccgtagtttgtctcctgggactgggggaggagtccgaggtggggttggttacctgtcccgaggggaccctgtccgggtccttgctcggagagtacggcctgatggctctgtgcagtacctggttgagtggggaggagggggcatcttctgaacagcctgcctctgcccagctccccattcacacacaccggcactttcataccctgacctctgacctcacctacagctgggatgtacctggagagatagggggtagttctccctactgcccaggctggaatccaagagtggggagtggggaagaggccctcttctctaccctccttcatgattcctgacccctcccatccttcccatttcctttgatgttattttgttacagctttttaaatattttttaaaattatttaacccctgggggcagagactgaggagggaggatgataagggatcccggactctgtatgattgaaataaagagaaataaacaaa

SEQ ID NO: 71 - DNA, 3787 bp, Homo sapiens
gcggccgctcggcggccgggggtcccttcggtggggccgcggctccccgcccgccgcccccgcgcgtccattcgctttgtgtcccgcgcgcggccgggccccccgcgcactctcagccctgcgccccgcggcccggcgggcggctcccggcgcggccccagcagcccgcgccggcattgtgtggacgcgcccggccgcgagcgcgcgcgcgggccctgccgagcgcccccggccccgtccgctccggccgcggcgcccgcgcccgccgcccccgccgccctcgccgcgcggcccccggcccggcccggcccgacccgggcagcgcagcggcggggcgagcggcggcgcggcaacatggcgacggtgcccgtgtactgcgtctgccggctgccctacgacgttacccgctttatgatcgagtgcgatgcctgcaaggactggttccacggcagctgtgttggggtggaagaggaagaggcaccagacatcgacatttaccactgcccgaactgcgagaaaacccatggcaagtccacactcaagaaaaagcggacttggcacaaacacggccctgggccaacaccggacgtgaaaccagtgcagaatggcagtcagctgttcatcaaggagctgcggagccgaaccttccccagtgctgaagacgtggtgtcccgtgtgccaggtagccagctcaccgtgggctacatggaggagcatggcttcactgagcccatccttgtccccaagaaagatggcctgggcttagctgtccctgccccaacattctacgtgagtgacgtcgagaactacgtggggccggaacggagtgtggatgtgacagatgtcaccaagcagaaggactgcaagatgaagctgaaggagtttgtggactattactacagcaccaaccgcaagcgggtcctcaacgtcaccaacctcgagttctctgacacccgaatgtccagcttcgtggagccacctgacattgtaaagaaactgtcatgggtagaaaactactggccagatgatgcattgctggccaagcccaaagtgaccaagtactgcctaatctgcgtgaaggacagttacaccgacttccacatcgactctgggggcgcctctgcctggtaccacgtgctcaagggggagaagaccttctatctcatcaggccggcctcggccaacatctccctgtatgagcgctggcggtctgcctctaaccacagcgagatgttctttgctgaccaggtcgacaaatgctacaagtgcatcgtcaagcagggccagaccctcttcatcccctcaggctggatctacgccacactcacccctgtggactgcctggccttcgcgggacatttcctccacagcctgagtgtggagatgcagatgagagcatacgaggtggaaaggaggttgaaacttggcagcctgactcagtttcccaactttgaaactgcgtgctggtacatgggaaagcacctattggaggcgttcaaaggttctcacaagtctgggaagcagctgcccccacatctagtccaaggagctaaaattctcaatggtgctttccgatcgtggacgaagaagcaggctttggcagagcatgaggacgagctcccggagcacttcaaaccttcacagctaatcaaggacctggccaaagagatccggctcagtgagaatgcctccaaagccgtccgaccggaagtgaatactgtcgcctcgtcagatgaggtgtgtgacggggaccgggagaaggaggagcccccgtctcccattgaggccaccccgcctcaatccctcctggagaaagtgtccaaaaaaaagactcccaaaactgtgaagatgcccaagccatccaaaatccccaagcccccgaagccccctaagcccccaaggccccccaaaacgctgaagctcaaagatggaggcaagaagaaagggaagaagtcccgggagtcagcctcacccaccatccccaacctggacctgctcgaagcccacaccaaggaggcactgaccaagatggagccgcccaagaagggcaaggccacaaagagtgtcctgagtgtgcccaacaaagatgtggttcacatgcagaatgatgtggagaggctggaaattcgagagcaaacaaagagcaagtcagaagccaagtggaaatacaagaacagcaaacctgactcgttactgaagatggaggaggagcagaggctggagaagtcgcccctggctgggaacaaggacaagttttccttttctttctccaacagaaaactcctgggctccaaggccctcaggcccccgagcagccctggtgtgttcggcgccttgcagagcttcaaggaggacaaggccaagcccgtgcgcgatgagtatgagtacgtatcagatgatggggagctgaagatagacgagtttcccatcaggaggaagaagagcgcccccaaaagggacttgtccttcttgttagacaagaaggaggctctcctcatgcccacctcgaagccaaagctggattctgcggtgtacaagagcgatgactcctctgacgagggctctctgcacatcgacacggacaccaagccaggcagaaatgccaaagtgaagaaggagagtgggagctccgcggccggcatcctggacctgctgcaggccagcgaggaggttggcgcactcgagtacaaccccaacagccagccccctgcctcccccagcacacaggaagccattcagggaatgctctccatggccaatctgcaggcctctgactcttgcctgcagaccacatggggcacggggcaggccaagggtggctcactggcagcccatggtgcccggaagattggtggtggcaacaaaggcacaggcaagcgcctgctgaagaggactgccaagaacagtgtggatctggaggactacgaggagcaggatcacctggatgcctgcttcaaggactcagactatgtttacccctcactggagtctgacgaagataaccccgtcttcaagtcccggtcaaagaagaggaaaggctcagacgatgctccgtacagccccacagccagggtcggtccatcggtgccaagacaagacaggcctgtgcgtgaggggaccagagtggcctccattgagacggggctggcagctgctgcagccaagctgtcccagcaggaggagcagaaaaacaggaagaagaagaacaccaaaaggaagccggctcctaacactgcctccccctccatctccacctctgcctccgcctccacgggtaccacctcggcctccaccaccccagcatccaccaccccggcctccaccaccccagcatccaccaccccggcctccaccagcacagccagcagccaggcctcacaggagggcagctcacctgagcccccacctgaatcacacagcagtagcctggctgaccacgaatatacagcagccggcacattctcggggtcccaggctggccgtgcctcccagcccatggcccctggagtctttctcacacagaggcggccttctgcatcatcccccaacaacactgctgccaaaggaaaacgtacaaaaaagggcatggccaccgccaagcaaaggcttggaaagatcttgaagatccatcggaatgggaaactgctcctctaaggcttggaaagccaggatccttctgatatgctaaggacccccggagccccgctacatcagcccctcccaggacggtggctgtgccgcctggcccggggagggcttgcttcattccgaccaattttccaatcaa

SEQ ID NO: 72 - DNA, 4058 bp, Homo sapiens
cccgccctcggcgcccccgcccctccagaaaggggaggaggcgaggggagcccgccgcggaggccgagcgagccggcgcccagcccagcctggcgactggggaccccggcacatgaggtggacgccccggggaagacttgggtgcacagccaggcgagaaggtcttgagtcagacagagcaccagccttggggaccctggaccactatcatggagactgagagcgagcagaactccaattccaccaatgggagttctagctcagggggcagctctcggccccagatagctcaaatgtcactttatgaacgacaagcagtgcaggctctgcaagcactgcagcggcagcccaatgcagctcagtatttccaccagttcatgctccagcagcagctcagtaatgcccagctgcatagcctggctgccgtccagcaggccacaattgctgccagtcggcaggccagctccccaaacaccagcactacacagcagcagactaccaccacccaggcctcgatcaatctggccaccacatcggccgcccagctcatcagccgatcccagagtgtgagctctcccagtgctaccaccttgacccaatctgtgctactggggaacaccacctccccacccctcaaccagtctcaggcccagatgtatctacggccacagctgggaaacctattgcaggtaaaccgaacccctgggtcgaatgtgcctctagcctcccaactcatcctgatgcctaatggggcggtggctgcagtccagcaggaggtgccatctgctcagtctcctggagttcatgcagatgcagatcaggttcagaacttggcagtaaggaatcaacaggcctcagctcaaggacctcagatgcaaggctccactcagaaggccattcctccaggagcctcccctgtctctagcctctcccaggcctctagccaggccctagcggtggcacaggcttcctctggggccacaaaccagtccctcaaccttagtcaagctggtggaggcagtgggaatagcatcccagggtccatgggtccaggtggaggtgggcaggcacatggtggtttgggtcagttgccttcctcaggaatgggtggtgggagctgtcccaggaagggtacaggagtggtgcagcccttgcctgcagcccaaacagtaactgtgagccagggcagccagacagaggcagaaagtgcagcagccaagaaggcagaagcagatgggagtggccagcagaatgtgggcatgaacctgacacggacagccacacctgcgcccagccagacacttattagctcagccacctacacacagatccagccccattcactgattcagcaacagcaacagatccacctccagcagaaacaggtggtgatccagcagcagattgccatccaccaccagcagcagttccagcaccggcagtcccagctccttcacacagctacacacctccagttggcgcagcagcagcagcagcaacaacagcaacagcagcaacagcagcagccgcaagccaccaccctcactgcccctcagccaccacaggtcccacctactcagcaggtcccaccttcccagtcccagcagcaagcccaaaccctggtcgttcagcccatgcttcagtcttcacccttgtctcttccacctgatgcagcccctaagccaccaattcccatccaatccaaaccacctgtagcacctatcaagccgcctcagttaggggccgctaagatgtcagctgcccagcaaccaccaccccatatccctgtgcaagttgtaggcactcgacagccaggtacagcccaggcacaggctttggggttggcacagctggcagctgctgtacctacttcccgggggatgccaggtacagtgcagtctggtcaggccctaaaggcttcctcgccaccttcatcccaggctcctggtgcactgcaggagtgccctcccacattggccccggggatgacccttgctcctgtgcaggggacagcacatgtggtaaagggtggggctaccacctccacacctgttgtagcccaggtccctgctgccttctatatgcagtctgtgcacttgccgggaaaaccccagacattggctgtcaaacgcaaggctgactctgaggaggagagagatgatgtctccacattgggttcaatgcttcctgccaaggcatctccagtagcagaaagcccaaaagtcatggacgagaagagcagtcttggagaaaaagctgaatcagtggctaatgtgaatgctaatactccaagcagtgaactagtagccttgacccccgccccttcagtaccgcctcctacactagccatggtgtctagacaaatgggtgactcaaaacccccacaggccatcgtgaagccccagattctcacccacatcattgaaggctttgttatccaggaaggagcagaacctttccccggtggttgttctcagttactgaaggagtctgagaagccactacagactggccttccgacaggcttgactgagaatcagtcaggtggcccctttggagtggacagcccatctgctgagttagataagaaggcgaatctcctgaagtgcgagtactgtgggaagtacgcccccgcagagcagtttcgtggctctaagaggttctgctccatgacctgcgctaagaggtacaatgtgagctgtagccatcagttccggctgaagaggaaaaaaatgaaagagtttcaagaagccaactatgctcgcgttcgcaggcgtggaccccgccgcagctcctctgacattgcccgtgccaagattcagggcaagtgccaccggggtcaagaagactctagccggggttcagataattccagttatgatgaagcactctctccaacatctcctgggcctttatcagtaagagctgggcatggagaacgtgacctggggaatcccaatacagctccacctacaccggaattacatggcatcaaccctgtgttcctgtccagtaatcccagccgttggagtgtagaggaggtgtacgagtttattgcttctctccaaggctgccaagagattgcagaggaatttcgctcacaggagattgatggacaggcctttttattacttaaagaagaacatcttatgagtgccatgaacatcaagctgggccctgccctcaagatctgtgccaagatcaatgtcctcaaggagacctaaggtggccctcttgcacaaaccagcctaaggcagacactctccactgtccaggttataacctggtaccagcagactttgcagggaagaaagagttgttccaatcatgtaaccttctgtaggggattactgagacagggaagagaagtgcaagaattggttgctggtgctacatggcggcagctttgacattttctctgggttctactttattttttaaaatctttacagttctcaccatttcacgtaccttaatccaatctttataaaagaggcagtctagagaactaggactgctcagccttatcctggagtggagcatttagcccaggtcttaattctccaagaggaggaatacatagtatggtaaggcaaggaactgggtggaatgtcaggttgcctgcccaatgggagaggtagggtttttctagcttgtgtgacagaagtagcaaaatctggtcctcccccctcccagtgtagctgtggctcagagttttttctttttgttgtcacttactcccttgtgattgaattttttctcctgcatccatggcaggatccccagccagtatagagacttggttggcatcttctgctgcagggactaaaagtatttgactggggcacatgtggctgttgtcattctttctgcatcccactgttcccctccaatttatgttattttctaccctgtttttcagttccatctctgctctgtcctatagctttataaaaccagagtgtgtggggctgaggtcaggagtataagtacctgccttaggcactattccttatataacaaaaatattaaatatttttttcctcagtaaaaggatg

SEQ ID NO: 73 - DNA, 2555 bp, Homo sapiens
ggcgccgcatgtgtctccgcggcggctgcagccctcgagcgcccgccgccgcgccccaaccccggccgccgcccgccctcccgccccggcctcgcgcccccgtcccggcctcgcgccccggccgccctttgttgacgccggccaggccgtgcggtcggatgcgccgcggcagccccgggccccggctcggaggctcccggggcgagaggaggcggcccgccggccgggaccccgcgcgagtcggccccggccaggggctgcgtaggcccgcccggccaggcccagccgcctggacagagacagggcagggcattgttcatgcactgaccgacctcagcatccccggcatgacctcagggaacggaaactctgcctccagcatcgccggcactgccccccagaatggtgagaataaaccaccacaggccattgtgaaaccccaaatcctgacgcatgttatcgaagggtttgtgatccaggagggggcggacgtttcccggtgggacgctcgtctgctggtggggaatctcaagaagaagtatgcacaggggttcctgcctgagaaacttccacagcaggatcacaccaccaccactgactcggagatggaggagccctatctgcaagaatccaaagaggagggtgctcccctcaaactcaagtgtgagctctgtggccgggtggactttgcctataagttcaagcgttccaagcgcttctgttccatggcttgtgcaaagaggtacaacgtgggatgcaccaaacgggtgggacttttccactcagaccggagcaagctgcagaaggcaggagctgcgacccacaaccgccgtcggccagcaaagccagtctgccaccacttaccaaggataccaagaagcagccaacaggcactgtgcccctttcggttactgctgctttgcgtaacacacagccaggaagactccagccgttgctcagataactcaagctatgaggaacccttgtcacccatctcagccagctcatctacttccgccggcgacaaggccagcgggacctggagctccccgacatgcatatgcgggacctggtgggcatgggacaccacttcctgccaagtgagccaccaagtgaatgtagaagacgtctacgaattcatccgctctctgccaggctgccaggagatagcagaggaattccgtgcccaggaaatcgacgggcaagccctgctgctgctcaaggaggaccacctgatgagcgttatgaacatcaagctggggcccgccctgaagatctacgcccgcatcagcatgctcaaggactcctagggctggtggcaccaggattctggcccagggcgcctcctcccgactgagcagagccagacagacattcctgaggggcccagaaatggcggcgttggagggcaggggctctccctaggggcatagctggtgaggaggtctgggcacctcctccatggctctcaggggcctttcatttctgtgggaggggcagagaggtaggtggcacagaagatggggctttatgcttgtaaatattgatagcactggcttcctccaaagtcccaatactctagccccgctctcttcccctctttctgtcccccattttccagggggtatatggtcagggctccccaacctgagttggttacttcaagggcagccagcaggcctggatggaggcctagaaagcccttgccttccttcctcccacttctttctccaggcctggttaactcttccgttgtcagcttctcccccttcagcctgtttctgcagcagccagggttctcccccctacaccctctgcaggtggagagagagaagctgggcccagccgcggtgcctgctggccaagacgccttaacgctgtgtgtatgactgtgtgactgtgtgggagcctggactgacagataggccaagggctactctctggcatctccaggtgttttgtagcaaacagccacttagtgctttgtcctggactccactcagcctcaggatggggaatagccaagaatggcagcctcagcgcagaggcaaggtcagaaagagacggcgcttcagagtttcctttccagacacccctccccgcactgtgaagttcccctgaccgccctcctggttcacaaagagcattaagaaagctgcggtggtctgagcaacatagcccagacgtggagcctcctggcctgcctgcccgcccaccctgggagtccagtggtgaggctcagagaacttctaaggggaaagaacagctggagtttctgttgatgtgaagaaggcagctcttggcctcccactcccacacttctttgcctataaatcttcctagcagcaatttgagctacctgaggaggaggcagggcagaagggcaagggcctgcctctgacctgccgtgtcctttgcaggaaggaggtaggcacctttctgagcttattctattccccacccacacccccaggcagggttggaaatgaaggacttttttaacctttgttttgttttttaaaaataaatctgtaaaatctgaaaaaaaaaaaaaa

SEQ ID NO: 74 - DNA, 766 bp, Homo sapiens
cactttgtcaccaactgctgccaactcgccaccactgctgccgcaatcgcaaccactgctttgtctctgaagtgagactgctcctggtgccatgaacggagacgacacctttgcaaagagacccagggatgatgctaaagcatcagagaagagaagcaaggcctttgatgatattgccacatacttctctaagaaagagtggaaaaagatgaaatactcggagaaaatcagctatgtgtatatgaagagaaactataaggccatgactaaactaggtttcaaagtcaccctcccacctttcatgtgtaataaacaggccacagacttccaggggaatgattttgataatgaccataaccgcaggattcaggttgaacatcctcagatgactttcggcaggctccacagaatcatcccgaagatcatgcccaagaagccagcagaggacgaaaatgattcgaagggagtgtcagaagcatctggcccacaaaacgatgggaaacaactgcaccccccaggaaaagcaaatatttctgagaagattaataagagatctggacccaaaagggggaaacatgcctggacccacagactgcgtgagagaaagcagctggtgatttatgaagagatcagtgaccctgaggaagatgacgagtaactcccctgggggatacgacacatgcccttgatgagaagcagaacgtggtgacctttcacgaacatgggcatggctgcggctccctcgtcatcaggtgcatagcaagtg

SEQ ID NO: 75 - DNA, 766 bp, Homo sapiens
ctctctttcgattcttccatactcagagtacgcacggtctgattttctctttggattcttccaaaatcagagtcagactgctcccggtgccatgaacggagacgacgcctttgcaaggagacccacggttggtgctcaaataccagagaagatccaaaaggccttcgatgatattgccaaatacttctctaaggaagagtgggaaaagatgaaagcctcggagaaaatcttctatgtgtatatgaagagaaagtatgaggctatgactaaactaggtttcaaggccaccctcccacctttcatgtgtaataaacgggccgaagacttccaggggaatgatttggataatgaccctaaccgtgggaatcaggttgaacgtcctcagatgactttcggcaggctccagggaatctccccgaagatcatgcccaagaagccagcagaggaaggaaatgattcggaggaagtgccagaagcatctggcccacaaaatgatgggaaagagctgtgccccccgggaaaaccaactacctctgagaagattcacgagagatctggacccaaaaggggggaacatgcctggacccacagactgcgtgagagaaaacagctggtgatttatgaagagatcagcgaccctgaggaagatgacgagtaactcccctcagggatacgacacatgcccatgatgagaagcagaacgtggtgacctttcacgaacatgggcatggctgcggacccctcgtcatcaggtgcatagcaagtg

SEQ ID NO: 76 - DNA, 2511 bp, Homo sapiens
atgagtaaactgtcgtttcgggcgcgggcgctagacgcctcgaagccgctgccggttttccgctgtgaggatctgcccgacctgcacgaatacgcctcgataaacagggccgtgccgcagatgcccaccggaatggagaaggaagaggagtcggaacatcatcttcagcgggctatttcagcacagcaggtgtatggcgagaagagggataatatggttataccggtcccagaggcagaaagtaatattgcttactatgagtctatatatcctggggaatttaagatgccaaagcagctcattcacatacagccttttagtttggatgctgaacagcctgattatgatttggattctgaagatgaagtatttgtgaataaactgaaaaagaaaatggacatctgcccattgcaatttgaggagatgattgaccgcctagaaaaaggcagtggtcagcagccagtcagtctgcaggaagccaaactactgctaaaagaagatgatgaactaattagagaagtttatgaatattggattaaaaagagaaaaaactgtcgagggccatctcttattccatcagtaaaacaagagaagcgagatggttccagcacaaatgatccttatgtggcttttagaaggcgtactgaaaaaatgcagactcgaaaaaatcgcaaaaatgatgaagcctcttacgaaaaaatgcttaagctgcgacgagatctaagtcgagctgttactattctagagatgataaaaagaagagaaaaaagtaaaagagagctattgcacttaacactggaaattatggaaaagaggtataatttgggcgactacaatggagagatcatgtctgaggttatggcacagagacagccaatgaaacctacttatgccatccccatcatccctattactaatagcagtcaatttaaacaccaggaagcaatggatgtgaaggagttcaaagttaataagcaagataaagccgatcttatccgaccgaaacggaaatatgaaaagaagcccaaagtcttaccatcgtctgccgctgctactccccaacagacgagtcctgctgcactgccagtcttcaatgctaaagatctgaatcagtatgactttcccagctcagacgaagaacctctctcccaggttttgtctggctcttcggaagctgaggaagacaatgatcctgatggtccttttgctttccgtaggaaagcaggctgtcagtactatgctcctcacttagaccaaactggcaactggccttggactagtcctaaagatggaggattaggggatgtgcgatatagatactgcttaactactctcaccgtaccccaaaggtgtattggatttgcacgaagacgggttgggcgcggtggaagggtcttactggacagagctcattcagactatgacagtgtgtttcaccatctggatttggaaatgctttcctcaccacaacattctccagtcaatcagtttgccaatacctcagaaacaaatacctcggacaaatctttctctaaagacctcagtcagatactagtcaatatcaaatcatgtagatggcggcattttaggcctcggacaccatccctacatgacagtgacaatgatgaactctcctgtagaaaattatataggagtataaaccgaacaggaacagcacaacctgggacccagacatgcagtacctctacgcaaagtaaaagtagcagtggttcagcacactttgcatttacagccgaacaataccagcaacatcaacagcaactggcactcatgcagaaacagcagcttgcacaaattcagcaacagcaagcaaatagtaattcctccaccaacacatcacagaaccttgcatctaaccagcagaaaagtggctttcgcctgaatatacagggtttagaaagaacactacagggttttgtttctaagactttggattctgctagtgcacagtttgctgcttctgctttggtgacatcagaacaactgatgggattcaagatgaaggatgatgtggtgcttggaatcggggtgaatggcgtccttccagcctcaggagtatacaagggcttacacctcagtagtactacaccaacagcacttgtacatacaagtccatcaacggcaggttcagctttgttacagccttcaaatattacacagacttcaagttcccacagtgcactgagtcatcaagtaactgctgccaattctgcaacaactcaggttctgattgggaacaacattcgattaactgtaccttcatcagttgccactgtaaactctattgccccaataaatgcacgacatatacctaggactttaagtgctgttccatcatctgccttaaagctggccgctgcagcaaactgtcaagtttccaaggtcccatcttcatcctctgtagattcagttccaagggaaaatcatgaatcagaaaagccagcactgaacaacatagcagacaacacagtagcgatggaggtgacgtag

SEQ ID NO: 77 - DNA, 2594 bp, Homo sapiens
gaggctggacacctgttctgctgttgtgtcctgccattctcctgaagaacagaggcacactgtaaaacccaacacttccccttgcattctataagattacagcaagatggaaataccaaatccccctacctccaaatgtatcacttactggaaaagaaaagtgaaatctgaatacatgcgacttcgacaacttaaacggcttcaggcaaatatgggtgcaaaggctttgtatgtggcaaattttgcaaaggttcaagaaaaaacccagatcctcaatgaagaatggaagaagcttcgtgtccaacctgttcagtcaatgaagcctgtgagtggacacccttttctcaaaaagtgtaccatagagagcattttcccgggatttgcaagccaacatatgttaatgaggtcactgaacacagttgcattggttcccatcatgtattcctggtcccctctccaacagaactttatggtagaagatgagacggttttgtgcaatattccctacatgggagatgaagtgaaagaagaagatgagacttttattgaggagctgatcaataactatgatgggaaagtccatggtgaagaagagatgatccctggatccgttctgattagtgatgctgtttttctggagttggtcgatgccctgaatcagtactcagatgaggaggaggaagggcacaatgacacctcagatggaaagcaggatgacagcaaagaagatctgccagtaacaagaaagagaaagcgacatgctattgaaggcaacaaaaagagttccaagaaacagttcccaaatgacatgatcttcagtgcaattgcctcaatgttccctgagaatggtgtcccagatgacatgaaggagaggtatcgagaactaacagagatgtcagaccccaatgcacttccccctcagtgcacacccaacatcgatggccccaatgccaagtctgtgcagcgggagcaatctctgcactccttccacacacttttttgccggcgctgctttaaatacgactgcttccttcacccttttcatgccacccctaatgtatataaacgcaagaataaagaaatcaagattgaaccagaaccatgtggcacagactgcttccttttgctggaaggagcaaaggagtatgccatgctccacaacccccgctccaagtgctctggtcgtcgccggagaaggcaccacatagtcagtgcttcctgctccaatgcctcagcctctgctgtggctgagactaaagaaggagacagtgacagggacacaggcaatgactgggcctccagttcttcagaggctaactctcgctgtcagactcccacaaaacagaaggctagtccagccccacctcaactctgcgtagtggaagcaccctcggagcctgtggaatggactggggctgaagaatctctttttcgagtcttccatggcacctacttcaacaacttctgttcaatagccaggcttctggggaccaagacgtgcaagcaggtctttcagtttgcagtcaaagaatcacttatcctgaagctgccaacagatgagctcatgtacccctcacagaagaagaaaagaaagcacagattgtgggctgcacactgcaggaagattcagctgaagaaagataactcttccacacaagtgtacaactaccaaccctgcgaccacccagaccgcccctgtgacagcacctgcccctgcatcatgactcagaatttctgtgagaagttctgccagtgcaacccagactgtcagaatcgtttccctggctgtcgctgtaagacccagtgcaataccaagcaatgtccttgctatctggcagtgcgagaatgtgaccctgacctgtgtctcacctgtggggcctcagagcactgggactgcaaggtggtttcctgtaaaaactgcagcatccagcgtggacttaagaagcacctgctgctggccccctctgatgtggccggatggggcaccttcataaaggagtctgtgcagaagaacgaattcatttctgaatactgtggtgagctcatctctcaggatgaggctgatcgacgcggaaaggtctatgacaaatacatgtccagcttcctcttcaacctcaataatgattttgtagtggatgctactcggaaaggaaacaaaattcgatttgcaaatcattcagtgaatcccaactgttatgccaaagtggtcatggtgaatggagaccatcggattgggatctttgccaagagggcaattcaagctggcgaagagctcttctttgattacaggtacagccaagctgatgctctcaagtacgtggggatcgagagggagaccgacgtcctttagccctcccaggccccaacggcagcacttatggtagcggcactgtcttggctttcgtgctcacaccactgctgctcgagtctcctgcactgtgtctcccacactgagaaaccccccaacccactccccctgtagtgaggcctctgccatgtccagagggcacaaaactgtctcaatgagaggggagacagaggcagctagggcttggtctcccaggacagagagttacagaaatgggagactgttt

SEQ ID NO: 78 - DNA, 2576 bp, Homo sapiens
gaattccgggcgacgcgcgggaacaacgcgagtcggcgcgcgggacgaagaataatcatgggccagactgggaagaaatctgagaagggaccagtttgttggcggaagcgtgtaaaatcagagtacatgcgactgagacagctcaagaggttcagacgagctgatgaagtaaagagtatgtttagttccaatcgtcagaaaattttggaaagaacggaaatcttaaaccaagaatggaaacagcgaaggatacagcctgtgcacatcctgacttctgtgagctcattgcgcgggactagggagtgttcggtgaccagtgacttggattttccaacacaagtcatcccattaaagactctgaatgcagttgcttcagtacccataatgtattcttggtctcccctacagcagaattttatggtggaagatgaaactgttttacataacattccttatatgggagatgaagttttagatcaggatggtactttcattgaagaactaataaaaaattatgatgggaaagtacacggggatagagaatgtgggtttataaatgatgaaatttttgtggagttggtgaatgcccttggtcaatataatgatgatgacgatgatgatgatggagacgatcctgaagaaagagaagaaaagcagaaagatctggaggatcaccgagatgataaagaaagccgcccacctcggaaatttccttctgataaaattttggaggccatttcctcaatgtttccagataagggcacagcagaagaactaaaggaaaaatataaagaactcaccgaacagcagctcccaggcgcacttcctcctgaatgtacccccaacatagatggaccaaatgctaaatctgttcagagagagcaaagcttacactcctttcatacgcttttctgtaggcgatgttttaaatatgactgcttcctacatccttttcatgcaacacccaacacttataagcggaagaacacagaaacagctctagacaacaaaccttgtggaccacagtgttaccagcatttggagggagcaaaggagtttgctgctgctctcaccgctgagcggataaagaccccaccaaaacgtccaggaggccgcagaagaggacggcttcccaataacagtagcaggcccagcacccccaccattaatgtgctggaatcaaaggatacagacagtgatagggaagcagggactgaaacggggggagagaacaatgataaagaagaagaagagaagaaagatgaaacttcgagctcctctgaagcaaattctcggtgtcaaacaccaataaagatgaagccaaatattgaacctcctgagaatgtggagtggagtggtgctgaagcctcaatgtttagagtcctcattggcacttactatgacaatttctgtgccattgctaggttaattgggaccaaaacatgtagacaggtgtatgagtttagagtcaaagaatctagcatcatagctccagctcccgctgaggatgtggatactcctccaaggaaaaagaagaggaaacaccggttgtgggctgcacactgcagaaagatacagctgaaaaaggacggctcctctaaccatgtttacaactatcaaccctgtgatcatccacggcagccttgtgacagttcgtgcccttgtgtgatagcacaaaatttttgtgaaaagttttgtcaatgtagttcagagtgtcaaaaccgctttccgggatgccgctgcaaagcacagtgcaacaccaagcagtgcccgtgctacctggctgtccgagagtgtgaccctgacctctgtcttacttgtggagccgctgaccattgggacagtaaaaatgtgtcctgcaagaactgcagtattcagcggggctccaaaaagcatctattgctggcaccatctgacgtggcaggctgggggatttttatcaaagatcctgtgcagaaaaatgaattcatctcagaatactgtggagagattatttctcaagatgaagctgacagaagagggaaagtgtatgataaatacatgtgcagctttctgttcaacttgaacaatgattttgtggtggatgcaacccgcaagggtaacaaaattcgttttgcaaatcattcggtaaatccaaactgctatgcaaaagttatgatggttaacggtgatcacaggataggtatttttgccaagagagccatccagactggcgaagagctgtttgttgattacagatacagccaggctgatgccctgaagtatgtcggcatcgaaagagaaatggaaatcccttgacatctgctacctcctccccctcctctgaaacagctgccttagcttcaggaacctcgagtactgtgggcaatttagaaaaagaacatgcagtttgaaattctgaatttgcaaagtactgtaagaataatttatagtaatgagtttaaaaatcaactttttattgccttctcaccagctgcaaagtgttttgtaccagtgaatttttgcaataatgcagtatggtacatttttcaactttgaataaagaatacttgaacttgaaaaaaaaaaaaaaaaaa

SEQ ID NO: 79 - DNA, 3203 bp, Homo sapiens
gagaggcagagatcggggcgagacaatggggatgtgggcgcgggagccccgttccggcttagcagcacctcccagccccgcagaataaaaccgatcgcgccccctccgcgcgcgccctcccccgagtgcggagcgggaggaggcggcggcggccgaggaggaggaggaggaggccccggaggaggaggcgttggaggtcgaggcggaggcggaggaggaggaggccgaggcgccggaggaggccgaggcgccggagcaggaggaggccggccggaggcggcatgagacgagcgtggcggccgcggctgctcggggccgcgctggttgcccattgacagcggcgtctgcagctcgcttcaagatggccgcttggctcgcattcattttctgctgaacgacttttaactttcattgtcttttccgcccgcttcgatcgcctcgcgccggctgctctttccgggattttttatcaagcagaaatgcatcgaacaacgagaatcaagatcactgagctaaatccccacctgatgtgtgtgctttgtggagggtacttcattgatgccacaaccataatagaatgtctacattccttctgtaaaacttgtattgttcgttacctggagaccagcaagtattgtcctatttgtgatgtccaagttcacaagaccagaccactactgaatataaggtcagataaaactctccaagatattgtatacaaattagttccagggcttttcaaaaatgaaatgaagagaagaagggatttttatgcagctcatccttctgctgatgctgccaatggctctaatgaagatagaggagaggttgcagatgaagataagagaattataactgatgatgagataataagcttatccattgaattctttgaccagaacagattggatcggaaagtaaacaaagacaaagagaaatctaaggaggaggtgaatgataaaagatacttacgatgcccagcagcaatgactgtgatgcacttaagaaagtttctcagaagtaaaatggacatacctaatactttccagattgatgtcatgtatgaggaggaacctttaaaggattattatacactaatggatattgcctacatttatacctggagaaggaatggtccacttccattgaaatacagagttcgacctacttgtaaaagaatgaagatcagtcaccagagagatggactgacaaatgctggagaactggaaagtgactctgggagtgacaaggccaacagcccagcaggaggagttccctccacctcttcttgtttgcctagccccagtactccagtgcagtctcctcatccacagtttcctcacatttccagtactatgaatggaaccagcaacagccccagcggtaaccaccaatcttcttttgccaatagacctcgaaaatcatcagtaaatgggtcatcagcaacttcttctggttgatacctgagactgttaaggaaaaaaattttaaacccctgatttatatagatatcttcagccattacgactttctagagctaatacatgtgactatcgtccaatttgctttcttttgtagtgacattaaatttggctataaaagatggactacatgtgatactcctgtccgtcttggttcaaaagaaagattgttgttataaagaattggtttcttggaaagcaggcaagactttttctctgtgttaggaaagatgggaaatggtttctgtaaccattgtttggatttggaagtactctgcagtggacataagcattgggccatagtttgttaatctcaactaacgcctacattacattctccttgatcgttcttgttattacgctgttttgtgaacctgtagaaaaacaagtgctttttatcttgaaattcaaccaacggaaagaatatgcatagaataatgcattctatgatgccatgtcactgtgaataacgatttcttgcagctatttagccattttgattgctgtttgatttatacttctctgttgctacgcaaaaccgatcaaagaaaagtgaacttcagttttacaatctgtatgcctaaaagcgggtactaccgtttattttactgacttgtttaaatgattcgcttttgtaagaatcagatggcattatgcttgttgtacaatgccatattggtatatgacataacaggaaacagtattgtatgatatatctataaatgctataaagaaatattgtgtttcatgcattcagaaatgattgttaaaattctcccaactggttcgacctttgcagatacccataacctatgttgagccttgcttaccagcaaagaatatttttaatgtggatatctaattctaaagtctgttccattagaagcaattggcacatctttctatactttatatacttttctccagtaatacatgtttactttaaaaattgttgcagtgaagaaaaacctttaactgagaaatatggaaaccgtcttaattttccattggctatgatggaattaatattgtattttaaaaatgcatattgatcactataattctaaaacaattttttaaataaaccagcaggttgctaaaagaaggcattttatctaaagttattttaataggtggtatagcagtaattttaaatttaagagttgcttttacagttaacaatggaatatgccttctctgctatgtctgaaaatagaagctatttattatgagcttctacaggtatttttaaatagagcaagcatgttgaatttaaaatatgaataaccccacccaacaattttcagtttattttttgctttggtcgaacttggtgtgtgttcatcagttatttgtgagggtgtttattctatatgaatattgtttcatgtttgtagggaaattgtagctaaacatttcattgtccccagtctgcaaaagaagcacaattctattgctttgtcttgcttatagtcattaaatcattacttttacatatattgctgttacttctgctttctttaaaaatatagtaaaggatgttttatgaagtcacaagatacatatatttttattttgacctaaatttgtacagtcccattgtaagtgttgtttctaattatagatgtaaaatgaaatttcatttgtaattggaaaaaatccaataaaaaggatattcatttagaaaatagctaagatctttaataaaaatttgatatgaaa

SEQ ID NO: 80 - DNA, 2227 bp, Homo sapiens
gagagcccgaacaggaagagggtacagctttgtgcaggtcacatgcccactgcagccctccagcctctggtccccagagcggactttggaagctgaactgcttttgttgctggaagacttatgttataatttaccctgggtggaccagggtcgtacaaaagggcaacgctccccagtccccccactcccgaccccggaatcatgcatcggactacacggatcaaaatcacagagctgaacccccacctcatgtgtgccctctgcggggggtacttcatcgacgccaccactatcgtggagtgcctgcattccttctgcaaaacctgcatcgtgcgctacctggagaccaacaaatactgccccatgtgtgacgtgcaggtccataaaacccggccgctgctgagcatcaggtctgacaaaacacttcaagacattgtctacaaattggtccctgggctttttaaagatgagatgaaacggcggcgggatttctatgcagcgtaccccctgacggaggtccccaacggctccaatgaggaccgcggcgaggtcttggagcaggagaagggggctctgagtgatgatgagattgtcagcctctccatcgaattctacgaaggtgccagggaccgggatgagaagaagggccccctggagaatggggatggggacaaagagaaaacaggggtgcgcttcctgcgatgcccagcagccatgaccgtcatgcatcttgccaagtttctccgcaacaagatggatgtgcccagcaagtacaaggtggaggttctgtacgaggacgagccactgaaggaatactacaccctcatggacatcgcctacatctacccctggcggcggaacgggcctctccccctcaagtaccgtgtccagccagcctgcaagcggctcaccctagccacggtgcccaccccctccgagggcaccaacaccagcggggcgtccgagtgtgagtcagtcagcgacaaggctcccagccctgccaccctgccagccacctcctcctccctgcccagcccagccaccccatcccatggctctcccagttcccatgggcctccagccacccaccctacctcccccactcccccttcgacagccagtggggccaccacagctgccaacgggggtagcttgaactgcctgcagacaccatcctccaccagcagggggcgcaagatgactgtcaacggcgctcccgtgccccccttaacttgaggccagggaccctctcccttcttccagccaagcctctccactccttccactttttctgggcccttttttccacttcttctactttccccagctcttcccaccttgggggtggggggcgggttttataaataaatatatatatatatgtacataggaaaaaccaaatatacatacttattttctatggaccaaccagattaatttaaatgccacaggaaacaaactttatgtgtgtgtgtatgtgtggaaaatggtgttcattttttttggggggggtcttgtgtaatttgctgtttttgggggtgcctggagatgaactggatgggccactggagtctcaataaagctctgcaccatcctcgctgtttcccaaggcaggtggtgtgttgggggccccttcagacccaaagctttaggcatgattccaactggctgcatataggagtcagttagaattgtttctttctctccccgtttctctccccatcttggctgctgtcctgcctctgaccagtggccgccccccgcgttgttgaatgtccagaaattgctaagaacagtgccttttacaaatgcagtttatccctggttctgaggagcaagtgcagggtggaggtggcacctgcatcacctcctcctcttgcagtggaaactttgtgcaaagaatagatagttctgcctcttttttttttttttcctgtgtgtgtggcctttgcatcatttatcttgtggaaaagaagattcaggccctgagaggtctcagctcttggaggagggctaaggctttagcattgtgaagcgctgcacccccaccaaccttaccctcaccggggaaccctcactagcaggactggtggtggagtctcacctggggcctagagtggaagtgggggtgggttaacctcacacaagcacagatcccagactttgccagaggcaaacagggaattccgccgatactgacgggctccaggagtcgtcgccacactcg

SEQ ID NO: 81 - DNA, 2635 bp, Homo sapiens
atcactggtctcgcgtgcgcgtgaccaggcccggtttccggtgccaggacctttccgaagcgtcgagtggcctaacggtcacagctgtcgcccatcggagaggcaggactactgcgagcagttttaccgcgacctccggaggccggcgtgacaggctctgtcactaaaataggaaccgaatattgtatctgacgcatcctgtaatactgaagagcaactgaagacagttgatgatgtccttattcattgccaggttatatatgatgctctgcaaaacctggataagaagattgatgtgattcgtagaaaggtttcaaaaatccaacgtttccatgcgagatccctgtggacaaatcataagcgatatggatataaaaagcattcttaccggcttgttaaaaagcttaaactccagaaaatgaagaaaaatgaggtttacgagacattctcctaccctgaaagttacagccccactttaccagtgtcaaggcgtgagaataattccccgagcaaccttccaaggccatccttttgcatggaagaataccagcgagctgagctggaggaggacccgatcctcagccgcactccgagtccagtgcatccctcagatttctctgagcataattgtcagccgtattatgcatctgatggtgcaacgtatggttcttcttcagggctctgccttggcaaccctcgggctgacagcatccacaacacttactcaactgaccatgcttctgcagcaccaccttcagttacaaggtcaccagttgaaaatgacggttacatagaggaaggaagcatcactaagcacccttcaacctggtcggtggaagcagtggtcctatttctaaaacaaacagatcctcttgcattatgccctcttgtcgacctcttcagaagccatgaaattgacgggaaggctctgctcctactcacgagtgacgtgttgctgaagcacttgggggtgaagctgggaacggctgtgaagctatgctactacattgaccgacttaaacaaggaaaatgctttgaaaattgaaaaaatccttgtgcaaatttagattgggccaacttctagaggcaccaatgccttcttagtgtggaatcatttttctgccctttagtcgtttttgttttgtagaaagtatctctcaaaatatattatagctagaattgtagaactatgttatagtccagtctacttctttaaaaaccatttaaactgctagatagtattagaatagtccaatagaaaattcattctttataggtctttaaaaattacttttattatattgtttacaaatatatttcatgcaagaaacagaaaaaaaaaaaaaccctttgattctggttcatctcgatacagagaaccaaaacagctaagagaggtattatcagggttgacaactcctatgattgaatctatgggaattattcctcagaagagaatttaaaggtgtacccatatatatctctttctggagtattttatctgtctgatgttgcagtattctacaagtttccagaaagagaatagccatataaattattttcctttctgctattatttctctatatgttttatttattcagatttagagtaaaaaataagcatataaacttttattatgtgctcttaacagttttaagataaactataggatagatagaatggttattttatgcaagaaatattgtaccgcaagggtggtttggatgaagtctgactactttttttcaaacaaactattatattaaaactgtcatattttggctaagtttggacctataactacactttcattgtttgcatctctctatgaagatacgtctgtccaaacttttaaaaggcataactgtattttatgtgtttattctttatatagatagtattttatattttattctcacccgaagtattcacacaatctttttaaaaaaaatttgaaatggcattttgtattgccacagaggtaggatgagccatatattagtgaaatgttttattttgtaaaatataaatggattatttgccatcattagtacctctcaacttactttttagaggacaagaaacaatctgtagattggtttccatacagggaagttctccgtcctatgcaatgtttctaattaatttgcttaattctgagccattaatcctgctacactttgaatgatacattaattcagactaatctttgggggctttattttgtaagttagaactttcaagggaaacatgttcaacactattattttgttataaatttataactttgttattacattgtgtaacaaatataaggtttacgagctatgagaattggtgctatcaccattagctatttgctgtaatgtcaagaaaatgttcaccagatgcaagaatgtaccttttctttttagaaagccaaatgtactttagacatgaatgcaactatttaaagaatagcttcatcaatgttattccttacatgtcataagattcttacttaaacttggtcttctttcaaattgtttgtatgaagatgctgtacccacttgaacagtcctcaggtgtttacataaatactatgttttacagttttcatattttaaaatattaataaagttaaatcacaatagt

SEQ ID NO: 82 - DNA, 4130 bp, Homo sapiens
gaagctcggtgccggctcgcgcgatcggtgggacagaatttcgttgttttcaccgacgagactggaggaaacaacaccaaatagggataccatgggacaaacagtgaatgaagattccatggatgtcaagaaggagaatcaagagaaaactcctcagtcaagtacatcttctgtacaaagggatgatttccactgggaggagtatttgaaagagactgggtctataagtgctccttcagagtgcttccgtcagtctcagattccacctgtgaatgatttcaaagttggtatgaaattggaagcccgtgaccctcgcaatgccacttcagtatgtattgctacggttattggaattactggggccaggttacggttacgactggatggtagtgacaacagaaatgatttttggaggcttgtcgattccccagacatacaacctgttgggacatgtgaaaaggaaggagacttacttcaacctccactagggtaccagatgaatacatcctcctggccgatgttcctcttaaagacactaaatgggtctgaaatggcatctgccacattatttaagaaggaaccaccaaagcccccactaaataattttaaagtggggatgaaactggaagctattgacaaaaagaacccgtatctcatctgtcctgcgaccattggagatgttaaaggggatgaagttcatatcacatttgatggctggagtggagcttttgattactggtgcaagtatgattctcgagatattttcccagctgggtggtgtcgcctgacaggagatgtattacaacccccaggaactagtgttcctattgtaaagaatatagcaaaaacagagtcttctccttccgaagcaagccagcattcaatgcagtctccacagaaaactactctaatattaccaacacagcaggtcaggagatcaagtcgaattaaaccacctggacctactgcagtccccaaaaggagcagttctgttaaaaatatcacaccaaggaaaaaaggtccaaactcaggaaaaaaggaaaaacctttgcccgtgatatgttctacatctgcagcttctctaaaatcgctgaccagagaccgtggcatgttatataaagatgtcgcttctgggccatgtaaaatagtgatgtctacagtctgtgtctatgtaaacaaacatggaaactttggccctcatctggatcccaagagaatccagcagctgcctgaccacttcggcccgggcccggtgaatgtggtgcttcgccggattgtgcaggcctgtgtggattgtgcccttgaaactaaaactgtttttggatacctgaagccagataatcgtggaggagaagtgataactgcctcctttgatggggaaactcattccatccagctccctccagtgaacagtgcatcatttgctcttcgctttcttgagaacttctgccacagtctgcagtgtgataaccttttgagtagccagccttttagttcttccaggggtcatactcacagctctgcagagcatgataaaaatcagtcagcaaaagaagatgtaacagaaaggcaaagcaccaaacgatctcctcagcaaactgtaccatatgttgttcctctctctcctaagctccccaaaacaaaggagtatgcgtctgaaggagaaccattgtttgctgggggaagtgccattcccaaagaggagaatctttcagaagattctaagagctcatcactaaattcaggaaattatttgaatcctgcctgtagaaatcctatgtatattcatacttcagtctcccaggatttttctcgaagtgtgccaggcaccacaagttcaccactagttggggacatatcccccaagagcagtccccatgaagttaaattccaaatgcagaggaaaagtgaagctccaagttatatagctgtacctgatcccagtgtcctgaaacaaggcttctctaaggacccttcaacctggtctgtggatgaagtgatacagtttatgaaacatacagatcctcagatatcaggccccctcgccgacctcttcaggcaacatgaaattgatgggaaggctctgttcctactcaagagtgatgtgatgatgaagtatatggggctgaagctggggccagcattaaagctgtgttactacattgaaaagcttaaagaaggaaaatacagttaaaaaatgtgtaagtttagattggacataattctcaggtgtactgttaacattttaatttaaaagtatttctcttagcagtttttgttttgtagacagttcccataaaaatattttatcagaattgcagaactgtagtaacagttcagtcaactttgtttttttcctggagtcaccaaccagctttgggagacacagccgccactcccccagtctacttctttaaaaagcatttaacaggttagtattggcatattcaaattggcagttctttatgtctttttaaattttcattgtacagtttacaaatatacttaatgtagttaacagagaaaaacctttgattttggttaacctttatatctagaaccaaaacagctaaatcccaaaggggaaaatatcagggattgacaacttctataattaaatccatgagaatttttcctcactagagaatttaaaggtgcacctgtagatatcatcttttctcagatattttgtgtgatactctgtggtgttctgttcatgttctatcagtatatctagaaagggaatagccatataaattattttccttttattatttctctgtatgttgtatttgatcatatttaaaggaaaaaagcaagcttataagctttcatgaagtgttcttaccagtttttgataaattttttaaattataggatagaattgtcattttatgcaggagatatttatactacgagggttgtttggatggagtcagattaaattttttcagtgaaattcctattattttaaaactttccatattttcactacgcttggacatttaactaggcattcttttcttacatctctatatgaagatacctgtgtccaaaatttttgaagatatatattgtatgtgtttattcttcatatggtactttaccatatttatatattgttttatacctgtaggtttacacacaagtaaatctttttttcttgaatttaatctgggcactttgcactgccacaggggtgacgatggaactatgtatatagttaggatgttttgatttcggtaaaaaatatatgtcccatcgttgctatcaccagtacctctcagcttactcttcaggggatatgaaacaatctgtagattgggtttccataacagggaagtccgtcctaatgccaagtttctaattaatttgcttagttctgagccatttattctgctacactttgaaagatatattagttctgacttattgttggggctttattttatttttatttttttgagatggagtttcactcttgtgcccaggctggagtgcaatagctcgatctcggctcactgcaacctcgcctccctggttcaagcaattctcctgcctcagcgattacaggaatgcaccaccacgcccagctaattttgtagagatggggtttctccatgttggtcaggctggtctcaaactcctgacctcaggtgatccgcccacctcggccttgcaaaatgctgggattacaggcatgagccacctgcccagccatgtttggggctttattttataagttagaactttgaagaggaaatggtgctatatgtttattgttattactttgtgtaactttgatgtaatgtttataagctatgagaatcagttataaaagtattagccatttgttgtaaatgccaataaaatattcaccaggggcaagaatgtacattttctttttagaaaaccaaatgtactttagacatgaatgcaactatttaaagaatagcttcatttatgttattccttacatgtcataagattcttacttaaacttggtcttctttcaagttgtttgtatgaagatgctgtacccacttgaacagtcctcaggtgtttacataaatactatgttttacagttttcatattttaaaatattaataaagttaatcgcaacgattc

SEQ ID NO: 83 - DNA, 1529 bp, Homo sapiens
ggctgctgtttctaaaacccctttccctctaacccacaccacctttctactcactgatgccttcaggaagccataatggatggcacagagattgctgtttcccctcggtcactgcattcagaactcatgtgccctatctgcctggacatgctgaagaatacgatgaccaccaaggagtgcctccacagattctgctctgactgcattgtcacagccctacggagcgggaacaaggagtgtcctacctgccgaaagaagctggtgtccaagcgatccctacggccagaccccaactttgatgccctgatctctaagatctatcctagccgggaggaatacgaggcccatcaagaccgagtgcttatccgcctgagccgcctgcacaaccagcaggcattgagctccagcattgaggaggggctacgcatgcaggccatgcacagggcccagcgtgtgaggcggccgataccagggtcagatcagaccacaacgatgagtgggggggaaggagagcccggggagggagaaggggatggagaagatgtgagctcagactccgcccctgactctgccccaggccctgctcccaagcgaccccgtggagggggcgcaggggggagcagtgtagggacagggggaggcggcactggtggggtgggtgggggtgccggttcggaagactctggtgaccggggagggactctgggagggggaacgctgggccccccaagccctcctggggcccccagccccccagagccaggtggagaaattgagctcgtgttccggccccaccccctgctcgtggagaagggagaatactgccagacgaggtatgtgaagacaactgggaatgccacagtggaccacctctccaagtacttggccctgcgcattgccctcgagcggaggcaacagcaggaagcaggggagccaggagggcctggagggggcgcctctgacaccggaggacctgatgggtgtggcggggagggtgggggtgccggaggaggtgacggtcctgaggagcctgctttgcccagcctggagggcgtcagtgaaaagcagtacaccatctacatcgcacctggaggcggggcgttcacgacgttgaatggctcgctgaccctggagctggtgaatgagaaattctggaaggtgtcccggccactggagctgtgctatgctcccaccaaggatccaaagtgaccccaccaggggacagccagaggaaggggaccatggggtatccctgtgtcctggtctatcaccccagcttctttgtcccccagtacccccagcccagccagccaataagaggacacaaatgaggacacgtggcttttatacaaagtatctatatgagattcttctatattgtacagagtggggcaaaacacgcccccatctgctgccttttctattgccctgcaacgtcccatctatacgaggtgttggagaaggtgaagaaccctcccattcacgcccgcctaccaacaacaaacgtgcttttttcctctttgaaaaaa

SEQ ID NO: 84 - DNA, 1036 bp, Homo sapiens
cccggacggcgtttctcctccgagcggcgccggtttcggcttggggggggcggggtacagcccatccatgaccatgggcgacaagaagagcccgaccaggccaaaaagacaagcgaaacctgccgcagacgaagggttttgggattgtagcgtctgcaccttcagaaacagtgctgaagcctttaaatgcagcatctgcgatgtgaggaaaggcacctccaccagaaaacctcggatcaattctcagctggtggcacaacaagtggcacaacagtatgccaccccaccaccccctaaaaaggagaagaaggagaaagttgaaaagcaggacaaagagaaacctgagaaagacaaggaaattagtcctagtgttaccaagaaaaataccaacaagaaaaccaaaccaaagtctgacattctgaaagatcctcctagtgaagcaaacagcatacagtctgcaaatgctacaacaaagaccagcgaaacaaatcacacctcaaggccccggctgaaaaacgtggacaggagcactgcacagcagttggcagtaactgtgggcaacgtcaccgtcattatcacagactttaaggaaaagactcgctcctcatcgacatcctcatccacagtgacctccagtgcagggtcagaacagcagaaccagagcagctcggggtcagagagcacagacaagggctcctcccgttcctccacgccaaagggcgacatgtcagcagtcaatgatgaatctttctgaaattgcacatggaattgtgaaaactatgaatcagggtatgaaattcaaaacctccacctgcccatgctgcttgcatccctggagaatcttctgtggacatcgacctcttagtgatgctgccaggataatttctgcttgccatgggcatctggccaccaaggaatttcgcaccctgacgattactcttgacacttttatgtattccattgttttatatgattttcctaacaatcatttataattggatgtgctcctgaatctacttttaataaaaaaaaaaaa

SEQ ID NO: 85 - DNA, 11910 bp, Homo sapiens
  misc_feature (89)..(89): unknown nucleotide
atggcgcacagctgtcggtggcgcttccccgcccgacccgggaccaccgggggcggcggcggcggggggcgccggggcctagggggcgncccgcggcaacgcgtcccggccctgctgcttccccccgggcccccggtcggcggtggcggccccggggcgcccccctcccccccggctgtggcggccgcggcggcggcggcgggaagcagcggggctggggttccagggggagcggccgccgcctcagcagcctcctcgtcgtccgcctcgtcttcgtcttcgtcatcgtcctcagcctcttcagggccggccctgctccgggtgggcccgggcttcgacgcggcgctgcaggtctcggccgccatcggcaccaacctgcgccggttccgggccgtgtttggggagagcggcgggggaggcggcagcggagaggatgagcaattcttaggttttggctcagatgaagaagtcagagtgcgaagtcccacaaggtctccttcagttaaaactagtcctcgaaaacctcgtgggagacctagaagtggctctgaccgaaattcagctatcctctcagatccatctgtgttttcccctctaaataaatcagagaccaaatctggagataagatcaagaagaaagattctaaaagtatagaaaagaagagaggaagacctcccaccttccctggagtaaaaatcaaaataacacatggaaaggacatttcagagttaccaaagggaaacaaagaagatagcctgaaaaaaattaaaaggacaccttctgctacgtttcagcaagccacaaagattaaaaaattaagagcaggtaaactctctcctctcaagtctaagtttaagacagggaagcttcaaataggaaggaagggggtacaaattgtacgacggagaggaaggcctccatcaacagaaaggataaagaccccttcggtctcctcattaattctgaactggaaaagccccagaaagtccggaaagacaaggaaggaacacctccacttacaaaagaagataagacagttgtcagacaaagccctcgaaggattaagccagttaggattattccttcttcaaaaaggacagatgcaaccattgctaagcaactcttacagagggcaaaaaaagggggctcaaaagaaaattgaaaaagaagcagctcagctgcagggaagaaaggtgaagacacaggtcaaaaatattcgacagttcatcatgcctgttgtcagtgctatctcctcgcggatcattaagacccctcggcggtttatagaggatgaggattatgaccctccaattaaaattgcccgattagagtctacaccgaatagtagattcagtgccccgtcctgtggatcttctgaaaaatcaagtgcagcttctcagcactcctctcaaatgtcttcagactcctctcgatctagtagccccagtgttgatacctccacagactctcaggcttctgaggagattcaggtacttcctgaggagcggagcgatacccctgaagttcatcctccactgcccatttcccagtccccagaaaatgagagtaatgataggagaagcagaaggtattcagtgtcggagagaagttttggatctagaacgacgaaaaaattatcaactctacaaagtgccccccagcagcagacctcctcgtctccacctccacctctgctgactccaccgccaccactgcagccagcctccagtatctctgaccacacaccttggcttatgcctccaacaatccccttagcatcaccatttttgcctgcttccactgctcctatgcaagggaagcgaaaatctattttgcgagaaccgacatttaggtggacttctttaaagcattctaggtcagagccacaatacttttcctcagcaaagtatgccaaagaaggtcttattcgcaaaccaatatttgataatttccgaccccctccactaactcccgaggacgttggctttgcatctggtttttctgcatctggtaccgctgcttcagcccgattgttttcgccactccattctggaacaaggtttgatatgcacaaaaggagccctcttctgagagctccaagatttactccaagtgaggctcactctagaatatttgagtctgtaaccttgcctagtaatcgaacttctgctggaacatcttcttcaggagtatccaatagaaaaaggaaaagaaaagtgtttagtcctattcgatctgaaccaagatctccttctcactccatgaggacaagaagtggaaggcttagtagttctgagctctcacctctcacccccccgtcttctgtctcttcctcgttaagcatttctgttagtcctcttgccactagtgccttaaacccaacttttacttttccttctcattccctgactcagtctggggaatctgcagagaaaaatcagagaccaaggaagcagactagtgctccggcagagccattttcatcaagtagtcctactcctctcttcccttggtttaccccaggctctcagactgaaagagggagaaataaagacaaggcccccgaggagctgtccaaagatcgagatgctgacaagagcgtggagaaggacaagagtagagagagagaccgggagagagaaaaggagaataagcgggagtcaaggaaagagaaaaggaaaaagggatcagaaattcagagtagttctgctttgtatcctgtgggtagggtttccaaagagaaggttgttggtgaagatgttgccacttcatcttctgccaaaaaagcaacagggcggaagaagtcttcatcacatgattctgggactgatattacttctgtgactcttggggatacaacagctgtcaaaaccaaaatacttataaagaaagggagaggaaatctggaaaaaaccaacttggacctcggcccaactgccccatccctggagaaggagaaaaccctctgcctttccactccttcatctagcactgttaaacattccacttcctccataggctccatgttggctcaggcagacaagcttccaatgactgacaagagggttgccagcctcctaaaaaaggccaaagctcagctctgcaagattgagaagagtaagagtcttaaacaaaccgaccagcccaaagcacagggtcaagaaagtgactcatcagagacctctgtgcgaggaccccggattaaacatgtctgcagaagagcagctgttgcccttggccgaaaacgagctgtgtttcctgatgacatgcccaccctgagtgccttaccatgggaagaacgagaaaagattttgtcttccatggggaatgatgacaagtcatcaattgctggctcagaagatgctgaacctcttgctccacccatcaaaccaattaaacctgtcactagaaacaaggcaccccaggaacctccagtaaagaaaggacgtcgatcgaggcggtgtgggcagtgtcccggctgccaggtgcctgaggactgtggtgtttgtactaattgcttagataagcccaagtttggtggtcgcaatataaagaagcagtgctgcaagatgagaaaatgtcagaatctacaatggatgccttccaaagcctacctgcagaagcaagctaaagctgtgaaaaagaaagagaaaaagtctaagaccagtgaaaagaaagacagcaaagagagcagtgttgtgaagaacgtggtggactctagtcagaaacctaccccatcagcaagagaggatcctgccccaaagaaaagcagtagtgagcctcctccacgaaagcccgtcgaggaaaagagtgaagaagggaatgtctcggcccctgggcctgaatccaaacaggccaccactccagcttccaggaagtcaagcaagcaggtctcccagccagcactggtcatcccgcctcagccacctactacaggaccgccaagaaaagaagttcccaaaaccactcctagtgagcccaagaaaaagcagcctccaccaccagaatcaggtccagagcagagcaaacagaaaaaagtggctccccgcccaagtatccctgtaaaacaaaaaccaaaagaaaaggaaaaaccacctccggtcaataagcaggagaatgcaggcactttgaacatcctcagcactctctccaatggcaatagttctaagcaaaaaattccagcagatggagtccacaggatcagagtggactttaaggaggattgtgaagcagaaaatgtgtgggagatgggaggcttaggaatcttgacttctgttcctataacacccagggtggtttgctttctctgtgccagtagtgggcatgtagagtttgtgtattgccaagtctgttgtgagcccttccacaagttttgtttagaggagaacgagcgccctctggaggaccagctggaaaattggtgttgtcgtcgttgcaaattctgtcacgtttgtggaaggcaacatcaggctacaaagcagctgctggagtgtaataagtgccgaaacagctatcaccctgagtgcctgggaccaaactaccccaccaaacccacaaagaagaagaaagtctggatctgtaccaagtgtgttcgctgtaagagctgtggatccacaactccaggcaaagggtgggatgcacagtggtctcatgatttctcactgtgtcatgattgcgccaagctctttgctaaaggaaacttctgccctctctgtgacaaatgttatgatgatgatgactatgagagtaagatgatgcaatgtggaaagtgtgatcgctgggtccattccaaatgtgagaatctttcagatgagatgtatgagattctatctaatctgccagaaagtgtggcctacacttgtgtgaactgtactgagcggcaccctgcagagtggcgactggcccttgaaaaagagctgcagatttctctgaagcaagttctgacagctttgttgaattctcggactaccagccatttgctacgctaccggcaggctgccaagcctccagacttaaatcccgagacagaggagagtataccttcccgcagctcccccgaaggacctgatccaccagttcttactgaggtcagcaaacaggatgatcagcagcctttagatctagaaggagtcaagaggaagatggaccaagggaattacacatctgtgttggagttcagtgatgatattgtgaagatcattcaagcagccattaattcagatggaggacagccagaaattaaaaaagccaacagcatggtcaagtccttcttcattcggcaaatggaacgtgtttttccatggttcagtgtcaaaaagtccaggttttgggagccaaataaagtatcaagcaacagtgggatgttaccaaacgcagtgcttccaccttcacttgaccataattatgctcagtggcaggagcgagaggaaaacagccacactgagcagcctcctttaatgaagaaaatcattccagctcccaaacccaaaggtcctggagaaccagactcaccaactcctctgcatcctcctacaccaccaattttgagtactgataggagtcgagaagacagtccagagctgaacccacccccaggcatagaagacaatagacagtgtgcgttatgtttgacttatggtgatgacagtgctaatgatgctggtcgtttactatatattggccaaaatgagtggacacatgtaaattgtgctttgtggtcagcggaagtgtttgaagatgatgacggatcactaaagaatgtgcatatggctgtgatcaggggcaagcagctgagatgtgaattctgccaaaagccaggagccaccgtgggttgctgtctcacatcctgcaccagcaactatcacttcatgtgttcccgagccaagaactgtgtctttctggatgataaaaaagtatattgccaacgacatcgggatttgatcaaaggcgaagtggttcctgagaatggatttgaagttttcagaagagtgtttgtggactttgaaggaatcagcttgagaaggaagtttctcaatggcttggaaccagaaaatatccacatgatgattgggtctatgacaatcgactgcttaggaattctaaatgatctctccgactgtgaagataagctctttcctattggatatcagtgttccagggtatactggagcaccacagatgctcgcaagcgctgtgtatatacatgcaagatagtggagtgccgtcctccagtcgtagagccggatatcaacagcactgttgaacatgatgaaaacaggaccattgcccatagtccaacatcttttacagaaagttcatcaaaagagagtcaaaacacagctgaaattataagtcctccatcaccagaccgacctcctcattcacaaacctctggctcctgttattatcatgtcatctcaaaggtccccaggattcgaacacccagttattctccaacacagagatcccctggctgtcgaccgttgccttctgcaggaagtcctaccccaaccactcatgaaatagtcacagtaggtgatcctttactctcctctggacttcgaagcattggctccaggcgtcacagtacctcttccttatcaccccagcggtccaaactccggataatgtctccaatgagaactgggaatacttactctaggaataatgtttcctcagtctccaccaccgggaccgctactgatcttgaatcaagtgccaaagtagttgatcatgtcttagggccactgaattcaagtactagtttagggcaaaacacttccacctcttcaaatttgcaaaggacagtggttactgtaggcaataaaaacagtcacttggatggatcttcatcttcagaaatgaagcagtccagtgcttcagacttggtgtccaagagctcctctttaaagggagagaagaccaaagtgctgagttccaagagctcagagggatctgcacataatgtggcttaccctggaattcctaaactggccccacaggttcataacacaacatctagagaactgaatgttagtaaaatcggctcctttgctgaaccctcttcagtgtcgttttcttctaaagaggccctctccttcccacacctccatttgagagggcaaaggaatgatcgagaccaacacacagattctacccaatcagcaaactcctctccagatgaagatactgaagtcaaaaccttgaagctatctggaatgagcaacagatcatccattatcaacgaacatatgggatctagttccagagataggagacagaaagggaaaaaatcctgtaaagaaactttcaaagaaaagcattccagtaaatcttttttggaacctggtcaggtgacaactggtgaggaaggaaacttgaagccagagtttatggatgaggttttgactcctgagtatatgggccaacgaccatgtaacaatgtttcttctgataagattggtgataaaggcctttctatgccaggagtccccaaagctccacccatgcaagtagaaggatctgccaaggaattacaggcaccacggaaacgcacagtcaaagtgacactgacacctctaaaaatggaaaatgagagtcaatccaaaaatgccctgaaagaaagtagtcctgcttcccctttgcaaatagagtcaacatctcccacagaaccaatttcagcctctgaaaatccaggagatggtccagtggcccaaccaagccccaataatacctcatgccaggattctcaaagtaacaactatcagaatcttccagtacaggacagaaacctaatgcttccagatggccccaaacctcaggaggatggctcttttaaaaggaggtatccccgtcgcagtgcccgtgcacgttctaacatgttttttgggcttaccccactctatggagtaagatcctatggtgaagaagacattccattctacagcagctcaactgggaagaagcgaggcaagagatcagctgaaggacaggtggatggggccgatgacttaagcacttcagatgaagacgacttatactattacaacttcactagaacagtgatttcttcaggtggagaggaacgactggcatcccataatttatttcgggaggaggaacagtgtgatcttccaaaaatctcacagttggatggtgttgatgatgggacagagagtgatactagtgtcacagccacaacaaggaaaagcagccagattccaaaaagaaatggtaaagaaaatggaacagagaacttaaagattgatagacctgaagatgctggggagaaagaacatgtcactaagagttctgttggccacaaaaatgagccaaagatggataactgccattctgtaagcagagttaaaacacagggacaagattccttggaagctcagctcagctcattggagtcaagccgcagagtccacacaagtaccccctccgacaaaaatttactggacacctataatactgagctcctgaaatcagattcagacaataacaacagtgatgactgtgggaatatcctgccttcagacattatggactttgtactaaagaatactccatccatgcaggctttgggtgagagcccagagtcatcttcatcagaactcctgaatcttggtgaaggattgggtcttgacagtaatcgtgaaaaagacatgggtctttttgaagtattttctcagcagctgcctacaacagaacctgtggatagtagtgtctcttcctctatctcagcagaggaacagtttgagttgcctctagagctaccatctgatctgtctgtcttgaccacccggagtcccactgtccccagccagaatcccagtagactagctgttatctcagactcaggggagaagagagtaaccatcacagaaaaatctgtagcctcctctgaaagtgacccagcactgctgagcccaggagtagatccaactcctgaaggccacatgactcctgatcattttatccaaggacacatggatgcagaccacatctctagccctccttgtggttcagtagagcaaggtcatggcaacaatcaggatttaactaggaacagtagcacccctggccttcaggtacctgtttccccaactgttcccatccagaaccagaagtatgtgcccaattctactgatagtcctggcccgtctcagatttccaatgcagctgtccagaccactccaccccacctgaagccagccactgagaaactcatagttgttaaccagaacatgcagccactttatgttctccaaactcttccaaatggagtgacccaaaaaatccaattgacctcttctgttagttctacacccagtgtgatggagacaaatacttcagtattgggacccatgggaggtggtctcacccttaccacaggactaaatccaagcttgccaacttctcaatctttgttcccttctgctagcaaaggattgctacccatgtctcatcaccagcacttacattccttccctgcagctactcaaagtagtttcccaccaaacatcagcaatcctccttcaggcctgcttattggggttcagcctcctccggatccccaacttttggtttcagaatccagccagaggacagacctcagtaccacagtagccactccatcctctggactcaagaaaagacccatatctcgtctacagacccgaaagaataaaaaacttgctccctctagtaccccttcaaacattgccccttctgatgtggtttctaatatgacattgattaacttcacaccctcccagcttcctaatcatccaagtctgttagatttggggtcacttaatacttcatctcaccgaactgtccccaacatcataaaaagatctaaatctagcatcatgtattttgaaccggcacccctgttaccacagagtgtgggaggaactgctgccacagcggcaggcacatcaacaataagccaggatactagccacctcacatcagggtctgtgtctggcttggcatccagttcctctgtcttgaatgttgtatccatgcaaactaccacaacccctacaagtagtgcgtcagttccaggacacgtcaccttaaccaacccaaggttgcttggtaccccagatattggctcaataagcaatcttttaatcaaagctagccagcagagcctggggattcaggaccagcctgtggctttaccgccaagttcaggaatgtttccacaactggggacatcacagaccccctctactgctgcaataacagcggcatctagcatctgtgtgctcccctccactcagactacgggcataacagccgcttcaccttctggggaagcagacgaacactatcagcttcagcatgtgaaccagctccttgccagcaaaactgggattcattcttcccagcgtgatcttgattctgcttcagggccccaggtatccaactttacccagacggtagacgctcctaatagcatgggactggagcagaacaaggctttatcctcagctgtgcaagccagccccacctctcctgggggttctccatcctctccatcttctggacagcggtcagcaagcccttcagtgccgggtcccactaaacccaaaccaaaaaccaaacggtttcagctgcctctagacaaagggaatggcaagaagcacaaagtttcccatttgcggaccagttcttctgaagcacacattccagaccaagaaacgacatccctgacctcaggcacagggactccaggagcagaggctgagcagcaggatacagctagcgtggagcagtcctcccagaaggagtgtgggcaacctgcagggcaagtcgctgttcttccggaagttcaggtgacccaaaatccagcaaatgaacaagaaagtgcagaacctaaaacagtggaagaagaggaaagtaatttcagctccccactgatgctttggcttcagcaagaacaaaagcggaaggaaagcattactgagaaaaaacccaagaaaggacttgtttttgaaatttccagtgatgatggctttcagatctgtgcagaaagtattgaagatgcctggaagtcattgacagataaagtccaggaagctcgatcaaatgcccgcctaaagcagctctcatttgcaggtgttaacggtttgaggatgctggggattctccatgatgcagttgtgttcctcattgagcagctgtctggtgccaagcactgtcgaaattacaaattccgtttccacaagccagaggaggccaatgaaccccccttgaaccctcacggctcagccagggctgaagtccacctcaggaagtcagcatttgacatgtttaacttcctggcttctaaacatcgtcagcctcctgaatacaaccccaatgatgaagaagaggaggaggtacagctgaagtcagctcggagggcaactagcatggatctgccaatgcccatgcgcttccggcacttaaaaaagacttctaaggaggcagttggtgtctacaggtctcccatccatggccggggtcttttctgtaagagaaacattgatgcaggtgagatggtgattgagtatgccggcaacgtcatccgctccatccagactgacaagcgggaaaagtattacgacagcaagggcattggttgctatatgttccgaattgatgactcagaggtagtggatgccaccatgcatggaaatcgtgcacgcttcatcaatcactcgtgtgagcctaactgctattctcgggtcatcaatattgatgggcagaagcacattgtcatctttgccatgcgtaagatctaccgaggagaggaactcacttacgactataagttccccattgaggatgccagcaacaagctgccctgcaactgtggcgccaagaaatgccggaagttcctaaactaa

SEQ ID NO: 86 - DNA, 1680 bp, Homo sapiens
atggacaatcagtgcaccgtccaggtgaggttagagctggggcatcgcgcccaactgcgcaagaagcccaccacggaggggttcactcacgactggatggtgtttgtccgcggccccgagcaatgtgacatccagcacttcgtggagaaggtggtcttctggctgcacgacagcttccccaagcccagacgcgtgtgcaaggagcccccctacaaagtagaggagtcggggtacgctggcttcatcatgcccatcgaggtgcacttcaaaaacaaggaggagccgaggaaggtctgcttcacctacgacctgttcctgaacctggaaggcaacccgcccgtgaaccacctgcgctgcgagaagctcaccttcaacaaccccaccacggagttccggtacaagctcctgcgggccggcggggtgatggtaatgcccgaaggagcagacacggtgtccaggcccagtcccgactaccccatgttacccacaattccactctctgccttctctgaccccaagaagaccaaaccatcccacggctccaaggacgccaacaaggagagcagcaagacctccaagccacacaaggtgaccaaggagcaccgggagcgcccccgcaaagactccgagagcaagagctcctccaaggagctggagcgtgagcaggccaaaagctccaaggacacctcgcggaagctgggcgagggccggctgcccaaggaggagaaggcgccaccgcccaaggctgccttcaaggaacccaagatggccctgaaagagaccaagctggaaagcacgtcccccaaccctgggcccccacccccacccccacccccaccccgggcttccagcaagcggccggccaccgccgactcgccaaagcccagcgccaagaagcagaagaagagcagctcgaaggggtcccggagtgctccaggcacctcgccccgcacctcctcctcctcctccttctcggacaagaagccggccaaggacaagagcagcaccagaggggagaaggtgaaggccgagagtgagccccgggaggccaaaaaggccctggaggtggaggagtccaactcagaggacgaggcctccttcaagtccgagtctgcccagtcaagcccgtccaactccagctccagctcagactccagctcagactcagacttcgagccatcccagaaccacagccaaggacccctgcgctccatggtggaggacctgcagtccgaggagtccgacgaggacgactcttcgtcaggcgaggaggctgccggcaagaccaacccggggagggactccaggttgagcttcagcgacagcgagagtgacaacagcgccgactcctccctgcccagccgtgagcccccacccccccagaagccacccccgcccaacagcaaggtgtcaggccggaggagccccgagtcctgcagcaagcctgagaagatcctcaagaagggcacctacgacaaggcctacacggatgagctggtggagctacaccggaggctgatggcgctgcgggagcgcaacgtgctgcagcagattgtgaatctgatcgaggagactggccacttcaatgtcaccaacaccaccttcgacttcgacctcttctccctggacgagaccaccgtgcgcaaactgcagagctgcctggaggccgtggccacatga

SEQ ID NO: 87 - DNA, 3171 bp, Homo sapiens
gggacagcttagggactatcgtcctgggactagggggaagttcgcgactttctgaagactggcaggaatgtgcctcctggccctcgatgcttcccccctgaggggaggcatcgtgagggactgtggcaggcttcactgaacgctgagccggggaggtccaactccacgtatggatccggggaatgagaattcagccacagaaggccgccgcgatcatagacctagatcccgacttcgaaccccagagccgtccccgctcctgcacctggccccttccccgaccagagatcgctaaccagccgtccgagccgcccgaggtggagccagatctgggggaaaaggtacacacggaggggcgctcagagccgatcctgttgccctctcggctctcagagccggccgggggcccccagcccggaatcctgggggctgtaacaggtcctcggaagggaggctcccgccggaatgcctggggaaatcagtcatatgcagaattcatcagccaggccattgaaagcgccccggagaagcgactgacacttgcccagatttacgagtggatggtccgtactgtaccctacttcaaggacaagggtgacagcaacagctcagcaggatggaagaactcgatccgccacaacctgtccctgcacagcaagttcatcaaggttcacaacgaggccaccggcaaaagctcttggtggatgctgaaccctgagggaggcaagagcggcaaagccccccgccgccgggccgcctccatggatagcagcagcaagctgctccggggccgcagtaaagcccccaagaagaaaccatctgtgctgccagctccacccgaaggtgccactccaacgagccctgtcggccactttgccaagtggtcaggcagcccttgctctcgaaaccgtgaagaagccgatatgtggaccaccttccgtccacgaagcagttcaaatgccagcagtgtcagcacccggctgtcccccttgaggccagagtctgaggtgctggcggaggaaataccagcttcagtcagcagttatgcagggggtgtccctcccaccctcaatgaaggtctagagctgttagatgggctcaatctcacctcttcccattccctgctatctcggagtggtctctctggcttctctttgcagcatcctggggttaccggccccttacacacctacagcagctcccttttcagcccagcagaggggcccctgtcagcaggagaagggtgcttctccagctcccaggctctggaggccctgctcacctctgatacgccaccaccccctgctgacgtcctcatgacccaggtagatcccattctgtcccaggctccgactcttctgttgctgggggggcttccttcctccagtaagctggccacgggcgtcggcctgtgtcccaagcccctagaggctcgaggccccagcagtctggttcccaccctttctatgatagcaccacctccagtcatggcaagtgcccccatccccaaggctctggggactcctgtgctcacaccccctactgaagctgcaagccaagacagaatgcctcaggatctagatcttgatatgtatatggagaacctggagtgtgacatggataacatcatcagtgacctcatggatgagggcgagggactggacttcaactttgagccagatccctgagtcatgcctggaagctttgtcccctgcttcagatgtggagccaggcgtgttcatatctactctttacccttgagccctccccaggaatttgggaccctgctttagagctagggtggggtctggtcacacacaggtgttgaagaaattataaagataaagctgccccatctggggacgatatggggagggagatgggaggggaaaggggagagggtttttctcactgtgccaattagggggtaaggccccctctcaggagccatcatcggctttccccattcctacccacttaggctttgtagcaagatgagcaatgctgttggaaatgtgaagtcaccagtggccttacccctgcctttgggagcaggatttttttgtagagagtcttatctgagctgagccaggctagctggagcctgggatttctatgcagtggccccttaggccagtgatgtgcggtgggtgggctgtttaggggatctggaagggccaaggtctgagcactggagtggctcgccaggccaaatcacccttagaaggctgcagataacagaaaggctttttataaacttttaaagaaatataaacacaaatatagagattttttaaccatggcagggtgctagtggtgggcagaatgcttttttttctttctgaaggctttgtgatagtgacatgatacaaacactacagacaataaatattaggagacacagggaagtggggagaggtggggagtaatagtaaacacagggaagagctcccctacggaccaggtatagagaaaggtctatgcagaaataggttagagtttccctaacaaaaaagctaacccaggtcccctcattccttcaacttgtgcctgggagtgtgtggtgttagggtgcagccacactcttctatgacccagcatgggttagtgctatggtgggagagtacattgaaggcctggaattagcttggggccagggaagggactgggaggggagagaagagaaggagggaaggatttaggatggtaaagttaggtacagagacctccctgttcaaggcccctgacagctgtccctgcccttcttccccttccctgactgcaggggttatgtggaagtgtgtgtggcagcaggcagcggggaggggaggaacagggaagggggagctggggagcttggctgagggtctgggaaatgagcagggatggggggggatgtggatcaggtttactagcacctgccagggaggccatctggggctccttctccaccccagcccccaaagcagcccttcccccagtgccctttgcatcgtcccctcccccacccctgctgtgggttcccatcatttcctgtgtcagcgcctggcctacccagattgtatcatgtgctagattggagtggggaagtgtgtcaaatcaataaatgaataaattcaataaatgcctataaccagcagaaaaaaaaaaaaaaaaaaaaaaaaaaaaaaa

SEQ ID NO: 88 - DNA, 3282 bp, Homo sapiens
atgaaggagatggtaggaggctgctgcgtatgttcggacgagaggggctgggccgagaacccgctggtctactgcgatgggcacgcgtgcagcgtggccgtccaccaagcttgctatggcatcgttcaggtgccaacgggaccctggttctgccggaaatgtgaatctcaggagcgagcagccagggtgaggtgtgagctgtgcccacacaaagacggggcattgaagaggactgataatggaggctgggcacacgtggtgtgtgccctctacatccccgaggtgcaatttgccaacgtgctcaccatggagcccatcgtgctgcagtacgtgcctcatgatcgcttcaacaagacctgttacatctgcgaggagacgggccgggagagcaaggcggcctcgggagcctgcatgacctgtaaccgccatggatgtcgacaagctttccacgtcacctgtgcccaaatggcaggcttgctgtgtgaggaagaagtgctggaggtggacaacgtcaagtactgcggctactgcaaataccacttcagcaagatgaagacatcccggcacagcagcgggggaggcggaggaggcgctggaggaggaggtggcagcatggggggaggtggcagtggtttcatctctgggaggagaagccggtcagcctcaccatccacgcagcaggagaagcaccccacccaccacgagaggggccagaagaagagtcgaaaggacaaagaacgccttaagcagaagcacaagaagcggcctgagtcgccccccagcatcctcaccccgcccgtggtccccactgctgacaaggtctcctcctcggcttcctcttcctcccaccacgaggccagcacgcaggagacctctgagagcagcagggagtcaaaggggaaaaagtcttccagccatagcctgagtcataaagggaagaaactgagcagtgggaaaggtgtgagcagttttacctccgcctcctcttcttcctcctcctcttcctcctcctctggggggcccttccagcctgcagtctcgtccctgcagagctcccctgacttctctgcattccccaagctggagcagccagaggaggacaagtactccaagcccacagcccccgccccttcagcccctccttctccctcagctcccgagccccccaaggctgacctttttgagcagaaggtggtcttctctggctttgggcccatcatgcgcttctccaccaccacctccagctcaggccgggcccgggcgccctcccctggggactataagtctccccacgtcacggggtctggggcctcggcaggcacccacaaacggatgcccgcactgagtgccacccctgtgcctgctgatgagacccctgagacaggcctgaaggagaagaagcacaaagccagcaagaggagccgccatgggccaggccgtcccaagggcagccggaacaaggagggcactgggggcccagctgccccatccttgcccagtgcccagctggctggctttaccgccactgctgcctcacccttctctggaggttccctggtcagctccggcctgggaggtctgtcctcccgaacctttgggccttctgggagcttgcccagcttgagcctggagtcccccttactaggggcaggcatctacaccagtaataaggaccccatctcccacagtggcgggatgctgcgggctgtctgcagcacccctctctcctccagcctcctggggcccccagggacctcggccctgccccgcctcagccgctccccgttcaccagcaccctcccctcctcttctgcttctatctccaccactcaggtgttttctctggctggctctacctttagcctcccttctacccacatctttggaacccccatgggtgccgttaatcccctcctctcccaagctgagagcagccacacagagccagacctggaggactgcagcttccggtgtcgggggacctcccctcaggagagtctgtcttccatgtcccccatcagcagcctccccgcactcttcgaccagacagcctctgcaccctgtgggggcggccagttagacccggcggccccagggacgactaacatggagcagcttctggagaagcagggcgacggggaggccggcgtcaacatcgtggagatgctgaaggcgctgcacgcgctgcagaaggagaaccagcggctgcaagagcagatcctgagcctgacggccaaaaaggagcggctgcagattctcaacgtgcagctctctgtgcccttccctgccctgcctgctgccctgcctgccgccaacggccctgtccctgggccctatggcctgcctccccaagccgggagcagcgactccttgagcaccagcaagagccctccgggaaagagcagcctcggcctggacaactcgctgtccacttcttctgaggacccacactcaggctgcccgagccgcagcagctcgtcgctgtccttccacagcacgcccccaccgctgcccctcctccagcagagccctgccactctgcccctggccctgcctggggcccctgccccactcccgccccagccgcagaacgggttgggccgggcacccggggcagcggggctgggggccatgcccatggctgaggggctgttgggggggctggcaggcagtgggggcctgcccctcaatgggctccttggggggttgaatggggccgctgcccccaaccccgcaagcttgagccaggctggcggggcccccacgctgcagctgccaggctgtctcaacagccttacagagcagcagagacatctccttcagcagcaagagcagcagctccagcaactccagcagctcctggcctccccgcagctgaccccggaacaccagactgttgtctaccagatgatccagcagatccagcagaaacgggagctgcagcgtctgcagatggctgggggctcccagctgcccatggccagcctgctggcaggaagctccaccccgctgctgtctgcgggtacccctggcctgctgcccacagcgtctgctccacccctgctgcccgctggagccctagtggctccctcgcttggcaacaacacaagtctcatggccgcagcagctgcagctgcagcagtagcagcagcaggcggacctccagtcctcactgcccagaccaaccccttcctcagcctgtcgggagcagagggcagtggcggtggccccaaaggagggaccgctgacaaaggagcctcagccaaccaggaaaaaggctaa

SEQ ID NO: 89 - DNA, 4839 bp, Homo sapiens
atgtcggcgggcggccgtgacgaggagcggcggaagctggccgacatcatccaccactggaacgccaaccggctggacctgttcgagatcagccagccgaccgaggatttggagttccatggagtgatgagattttattttcaagataaagctgctggaaactttgcaacaaaatgtattcgggtctctagtactgccaccactcaagatgtaatcgaaacgctcgcggagaaatttcgacctgatatgcgaatgctgtcctctcccaagtattcactctatgaagtgcatgtcagcggagaaagaagattggatatagatgagaaacctctagttgtacaactgaattggaacaaagatgatcgggaaggcagatttgttcttaagaatgagaatgacgccattcctcctaaggctcaaagtaatggacctgaaaagcaggaaaaagaaggggttatccagaacttcaagagaactctctcaaagaaagaaaagaaggaaaaaaagaagagagaaaaagaggcattgcgacaggcatctgataaagatgatagacctttccaaggggaggatgttgaaaattctcgactggctgctgaggtttacaaagacatgccggaaaccagctttactcgaaccatttctaatcctgaggtggttatgaaacgacggaggcagcaaaaattggaaaagagaatgcaggaatttcggagctcagatgggcggcctgattcaggtggaacattgagaatttatgcagatagtttaaaaccaaatattccctacaagacaatcctgctgtctactacagatcctgcagactttgctgtggctgaagctttagagaagtatggtctggaaaaagaaaaccctaaggattactgcatcgcccgggttatgcttcctcctggagcccagcattctgatgaaaagggtgctaaagaaattattcttgatgatgatgagtgtcctttacaaatcttcagggaatggccaagtgacaaagggattttagtctttcagttgaagaggaggccaccagaccacatcccaaagaaaaccaagaaacacttggaaggcaagacacccaagggaaaggagagagctgacgggtctgtctatggctccacccttcctccggagaagctgccctatttagtagagttaagcccagatggttctgactctagagataagccaaagctttaccgccttcagttaagtgttactgaagttgggacagaaaagttggatgacaactctatccagttgtttggcccaggaattcagccccatcactgtgaccttaccaacatggatggagtggtcactgtgacgcccagaagtatggacgcagaaacctacgtggaaggccagcgcatctcagaaaccaccatgctgcagagtggcatgaaagtgcagtttggggcgtcccatgtatttaagtttgtggaccccagtcaggatcatgctcttgcaaaaagatctgtggatggaggcctgatggttaagggcccaagacataaacctggaattgttcaggagacaacttttgatttgggaggagatattcatagtgggacagcattaccgacaagcaagagcaccactaggctggacagcgacagagtgtcgtctgcctctagcacagccgagcggggaatggtgaagccgatgatcagagtagaacagcagccagattatcgcaggcaagaaagcagaacacaggatgcttctgggcctgagctgatactacctgcaagcattgaattcagggaaagttctgaagattcatttttgtctgccattataaattatactaatagctctacagtccactttaagttgtcccctacatatgtattatatatggcatgccggtatgtattgtccaaccagtacagacctgacatcagccctacagagcgcacacataaagtcattgcagtcgtcaacaagatggtgagcatgatggagggtgtcatccagaaacagaagaatattgcaggggcacttgccttctggatggcaaatgcatctgaacttctcaacttcattaagcaagaccgagaccttagtcggatcacactggatgctcaagatgttttagcacatttggttcaaatggcatttaaatacttggttcactgtcttcaatcagaacttaataattacatgccagcctttctagatgaccctgaagagaacagtctgcaacgaccaaaaatagatgatgtgctgcacacgctcacaggagccatgtccttgctacgacgctgcagagtcaatgccgccctgaccatccagctcttctctcagctcttccacttcatcaatatgtggctgttcaatagattggtgaccgacccagattcggggctgtgctcccattactggggtgcgattatccgtcagcagttgggccatattgaagcctgggctgagaagcaggggctggaactggctgcggactgtcatctgagcaggatcgtgcaggcaacgactttgcttaccatggataagtatgcacctgatgacattccaaatataaacagcacctgctttaagttaaattcattacaacttcaagccttattacagaactatcactgtgcacctgatgagccttttatcccaacggatcttatagaaaatgtagtgactgtggctgaaaacactgccgatgagctggcccgcagtgatggaagggaagtgcagttggaggaggatcctgatctgcagctgccgtttcttttgccagaagatggttattcttgtgatgttgtcagaaacattccaaatggtttacaagaatttttagaccctctgtgccagagaggattttgcaggttaattcctcacacacgttcaccaggtacttggacaatatattttgaaggtgcagattatgaaagtcaccttctgcgtgagaacacagagctggctcagcctctgaggaaagaacctgaaataatcactgtgaccctaaaaaagcagaatggaatgggccttagcattgttgcagcaaagggtgctggtcaagataaactaggaatctacgtgaagtcggttgtgaaaggaggtgctgcagatgtggatggacgtctagctgcaggtgatcagctcctcagtgtggatggacgaagtctggttggactctctcaggaaagggcggcagaactcatgacaagaacaagctctgtggtgacactggaagtagcaaagcagggtgccatctaccacggtctggccacccttctcaatcagccatcccccatgatgcagagaatttcagatcgtcgtggctcaggtaaaccccgaccaaagagtgaaggctttgagctctataataattcaactcaaaatgggtctcctgagagtcctcagctgccttgggcagaatatagtgaaccaaagaaattgcctggtgatgacagactgatgaaaaatagagctgatcaccgttccagccccaacgtagcaaatcagcctcctagtcctggagggaaaagtgcatatgcctctggaacaacagcgaagataacatctgtctctactggaaacctctgcactgaggagcagacgcctccgcctagacctgaagcctaccccattcccactcagacgtacaccagagagtattttaccttcccagcttccaaatcccaggatcggatggctcctcctcagaaccagtggccaaattatgaggaaaagccacatatgcacacagatagtaatcattccagtattgcaattcagcgtgttacacgttcccaagaagaacttcgagaagataaagcttaccaacttgagcggcatcgaatagaggcagctatggaccgaaagtctgatagtgatatgtggataaatcagagctcctcactggactccagtacctctagccaggagcatctgaaccattcctctaagtcggtcacccctgcttccacactgaccaaaagtggccctggccgttggaaaacaccagcagccataccggccacccctgtggccgtctcccagccaatccgaacagacctgcctccgccacccccgccacctccagtccactatgccggtgatttcgatggaatgtccatggatttgcctctcccaccacccccttccgccaaccagatagggctgccgtctgcgcaggtggctgctgctgaacggagaaagagagaagaacatcagcgttggtatgagaaggagaaggcccccctggaggaggagcgggagaggaagcggagagagcaggagaggaagttgggccagatgcgcactcagtccttaaaccctgctccgttttctcccctgactgcacagcagatgaagcccgaaaagccttccacactccagcggccacaggaaacagtcattcgggagctgcagcctcagcagcagccccgcacgatcgagcgcagagacttgcagtacattacagtcagcaaagaggagctttcctcgggggacagtctgtcccccgacccgtggaagcgggacgccaaggagaagctggagaagcagcagcagatgcacatcgtggacatgctgagcaaggagatccaggagctccagagcaaaccggaccgcagcgccgaggagagcgaccggctgcgcaagctcatgctggagtggcagttccagaagagactccaggagtcgaagcagaaggacgaagatgacgaggaggaggaggacgatgatgtggacaccatgctgatcatgcagcgcctggaggctgaacgaagagcgagggtaaaggggggagtgctttggctgtgcccatctgtggtccctattttagcttctgcgtgtttcccatggggatag

SEQ ID NO: 90 - DNA, 3376 bp, Homo sapiens
tttggggctgagtttaataagcgagcgagcgagcaagcgagcgcggggggaaaaaggcagagaatgtccgccatctaccctccgctcctgggcgcgctctcattcatagcagcctcttcatgaattacagctgagggggggcggaggaggggggggtaccacacaacaccccagcaaacctccgggcccccaggcatggctagctcgtgttccgtgcaggtgaagctggagctggggcaccgcgcccaggtgaggaaaaaacccaccgtggagggcttcacccacgactggatggtgttcgtacgcggtccggagcacagtaacatacagcactttgtggagaaagtcgtcttccacttgcacgaaagctttcctaggccaaaaagagtgtgcaaagatccaccttacaaagtagaagaatctgggtatgctggtttcattttgccaattgaagtttattttaaaaacaaggaagaacctaggaaagtccgctttgattatgacttattcctgcatcttgaaggccatccaccagtgaatcacctccgctgtgaaaagctaactttcaacaaccccacagaggactttaggagaaagttgctgaaggcaggaggggaccctaataggagtattcataccagcagcagcagcagcagcagcagtagcagcagcagcagcagcagcagcagcagcagtagcagcagcagcagcagcagcagcagcagcagtagcagcagcagtagcagcagcagcagcagcagtagtaccagtttttcaaagcctcacaaattaatgaaggagcacaaggaaaaaccttctaaagactccagagaacataaaagtgccttcaaagaaccttccagggatcacaacaaatcttccaaagaatcctctaagaaacccaaagaaaataaaccactgaaagaagagaaaatagttcctaagatggccttcaaggaacctaaacccatgtcaaaagagccaaaaccagatagtaacttactcaccatcaccagtggacaagataagaaggctcctagtaaaaggccgcccatttcagattctgaagaactctcagccaaaaaaaggaaaaagagtagctcagaggctttatttaaaagtttttctagcgcaccaccactgatactcacttgttctgctgacaaaaaacagataaaagataaatctcatgtcaagatgggaaaggtcaaaattgaaagtgagacatcagagaagaagaaatcaacgttaccgccatttgatgatattgtggatcccaatgattcagatgtggaggagaatatatcctctaaatctgattctgaacaacccagtcctgccagctccagctccagctccagctccagcttcacaccatcccagaccaggcaacaaggtcctttgaggtctataatgaaagatctgcattctgatgacaatgaggaggaatcagatgaagtggaggataacgacaatgactctgaaatggagaggcctgtaaatagaggaggcagccgaagtcgcagagttagcttaagtgatggcagcgatagtgaaagcagttctgcttcttcacccctacatcacgaacctccaccacccttactaaaaaccaacaacaaccagattcttgaagtgaaaagtccaataaagcaaagcaaatcagataagcaaataaagaatggtgaatgtgacaaggcatacctagatgaactggtagagcttcacagaaggttaatgacattgagagaaagacacattctgcagcagatcgtgaaccttatagaagaaactggacactttcatatcacaaacacaacatttgattttgatctttgctcgctggacaaaaccacagtccgtaaactacagagttacctggaaacatctggaacatcctgaggatataacaactggatgcatcaagaactattgtgttttttttttttggttttttttttttttggttgtgattttttgttcttgttgtttatatgaaaacactcaaaatgatgcaaccaaaagggaaaaaataaaaatcaaacaaccttcagctttatttttctttaaagccagtcatcatctcttgataaaggagaggttaaagcaaaccagcctcagcggaccactcttctctccaaggaaatccccgggaagagttagcctggatagccttgaaaacaaacaaatcaaacacaacacaagaaaactcaaagaatgtgtatggtatcatgtatctctctgtggtggttcattccacaggacgaatgcatattcaacacactgccttattacataactgatctatttattatcgcatacagatattctaagtcgttgagggaatgacaccatcagacattataagtacttggtcccgtggatgctctttcaatgcagcacccttgccatcccaagcccagtgaccttactcgtataccgtgccactttccaccaactttttccaagtcctttaactcgttgcagtctgtattttccaccttttgtttttccagttccaggacacagattatcaactggggggaccaaatagccaccttgattttcttctttgtggtctttttcctgaaagttggggcccagtccttggctgtatccatgtaatgatcttggaccatggtagaaaatgcaccaaataggatcatatgaattgctgtctagccttagtcaataaacttgtaggacttttaaacaaaagtgtacctgtaaatgtcctgaatccagcattgttgagctgtcatcaacattcttgtgtctgttttactgttacaatattaggtgaatatggaagtaaaggcattccacaggatcatcatttaaaaaaaaagaattctggtcctgttttctaaaaaaaaaaaactgttgtagaaattcttaatttggatctatttattagtcagagtttcagctttcttcagctgccagtgtgttactcatctttatcctaaaaatctggaatcagagatttttgtttgttcacatatgattctcttagacacttttatatttgaaaaaattaaaatctttctttggggaaaaattcttggttattctgccataacagattatgtattaacttgtagattcagtggttcaatacctgtttagttgcttgctaatatttccagaaggatttcttgtattggtgaaagacggttggggatggggggatttttttgttcttgttgtacccttgttttgaaactagaaatctgtcctgtggcatgcaaaagaaagcaaattatttttaaaagaaaaaaaccaaagtacttttggtgtcattattccatcttctccataagtggagaaatgaaaagtaagaacagctcatcttcaaagtttttactagaaattc

SEQ ID NO: 91 - DNA, 9390 bp, Homo sapiens
ggcaatttcttttcctttctaactgtggcccgcgttgtgctgttgctgggcaggcgttgggcgccggcggtcttcgagcgtgggggcccgctggctttcccttctcagaaactgcgccgggggcgctcgcttgccccggattcggacgcggcgctccccgggctcgtctgaagtgcagatcgccgcagaggccccagtgcccggatgtccatcaggattagcgcgagccaatacgggccgagcccggggctgcgccgaggacgcccggggctcgagagcaggtagtcccgtaacatcggggcgccgcgccgggacgcgtccccgcccggctccgccaaatggtgagcgcggcgctggcagcagggcccgcggggtgaaggcgctcatggacggaagacccctggctctataagctgaattatggcagcccagtcaagtttgtacaatgacgacagaaacctgcttcgaattagagagaaggaaagacgcaaccaggaagcccaccaagagaaagaggcatttcctgaaaagattcccctttttggagagccctacaagacagcaaaaggtgatgagctgtctagtcgaatacagaacatgttgggaaactacgaagaagtgaaggagttccttagtactaagtctcacactcatcgcctggatgcttctgaaaataggttgggaaagccgaaatatcctttaattcctgacaaagggagcagcattccatccagctccttccacactagtgtccaccaccagtccattcacactcctgcgtctggaccactttctgttggcaacattagccacaatccaaagatggcgcagccaagaactgaaccaatgccaagtctccatgccaaaagctgcggcccaccggacagccagcacctgacccaggatcgccttggtcaggaggggttcggctctagtcatcacaagaaaggtgaccgaagagctgacggagaccactgtgcttcggtgacagattcggctccagagagggagctttctcccttaatctctttgccttccccagttccccctttgtcacctatacattccaaccagcaaactcttccccggacgcaaggaagcagcaaggttcatggcagcagcaataacagtaaaggctattgcccagccaaatctcccaaggacctagcagtgaaagtccatgataaagagacccctcaagacagtttggtggcccctgcccagccgccttctcagacatttccacctccctccctcccctcaaaaagtgttgcaatgcagcagaagcccacggcttatgtccggcccatggatggtcaagatcaggcccctagtgaatcccctgaactgaaaccactgccggaggactatcgacagcagacctttgaaaaaacagacttgaaagtgcctgccaaagccaagctcaccaaactgaagatgccttctcagtcagttgagcagacctactccaatgaagtccattgtgttgaagagattctgaaggaaatgacccattcatggccgcctcctttgacagcaatacatacgcctagtacagctgagccatccaagtttcctttccctacaaaggactctcagcatgtcagttctgtaacccaaaaccaaaaacaatatgatacatcttcaaaaactcactcaaattctcagcaaggaacgtcatccatgctcgaagacgaccttcagctcagtgacagtgaggacagtgacagtgaacaaaccccagagaagcctccctcctcatctgcacctccaagtgctccacagtcccttccagaaccagtggcatcagcacattccagcagtgcagagtcagaaagcaccagtgactcagacagttcctcagactcagagagcgagagcagttcaagtgacagcgaagaaaatgagcccctagaaaccccagctccggagcctgagcctccaacaacaaacaaatggcagctggacaactggctgaccaaagtcagccagccagctgcgccaccagagggccccaggagcacagagcccccacggcggcacccagagagtaagggcagcagcgacagtgccacgagtcaggagcattctgaatccaaagatcctccccctaaaagctccagcaaagccccccgggccccacccgaagccccccaccccggaaagaggagctgtcagaagtctccggcacagcaggagcccccacaaaggcaaaccgttggaaccaaacaacccaaaaaacctgtcaaggcctctgcccgggcaggttcacggaccagcctgcagggggaaagggagccagggcttcttccctatggctcccgagaccagacttccaaagacaagcccaaggtgaagacgaaaggacggccccgggccgcagcaagcaacgaacccaagccagcagtgcccccctccagtgagaagaagaagcacaagagctccctccctgccccctctaaggctctctcaggcccagaacccgcgaaggacaatgtggaggacaggacccctgagcactttgctcttgttcccctgactgagagccagggcccaccccacagtggcagcggcagcaggactagtggctgccgccaagccgtggtggtccaggaggacagccgcaaagacagactcccattgcctttgagagacaccaagctgctctcaccgctcagggacactcctcccccacaaagcttgatggtgaagatcaccctagacctgctctctcggataccccagcctcccgggaaggggagccgccagaggaaagcagaagataaacagccgcccgcagggaagaagcacagctctgagaagaggagctcagacagctcaagcaagttggccaaaaagagaaagggtgaagcagaaagagactgtgataacaagaaaatcagactggagaaggaaatcaaatcacagtcatcttcatcttcatcctcccacaaagaatcttctaaaacaaagccctccaggccctcctcacagtcctcaaagaaggaaatgctccccccgccacccgtgtcctcgtcctcccagaagccagccaagcctgcacttaagaggtcaaggcgggaagcagacacctgtggccaggaccctcccaaaagtgccagcagtaccaagagcaaccacaaagactcttccattcccaagcagagaagagtagaggggaagggctccagaagctcctcggagcacaagggttcttccggagatactgcaaatccttttccagtgccttctttgccaaatggtaactctaaaccagggaagcctcaagtgaagtttgacaaacaacaagcagaccttcacatgagggaggcaaaaaagatgaagcagaaagcagagttaatgacggacagggttggaaaggcttttaagtacctggaagccgtcttgtccttcattgagtgcggaattgccacagagtctgaaagccagtcatccaagtcagcttactctgtctactcagaaactgtagatctcattaaattcataatgtcattaaaatccttctcagatgccacagcgccaacacaagagaaaatatttgctgttttatgcatgcgttgccagtccattttgaacatggcgatgtttcgttgtaaaaaagacatagcaataaagtattctcgtactcttaataaacacttcgagagttcttccaaagtcgcccaggcaccttctccatgcattgcaagcacaggcacaccatcccctctttccccaatgccttctcctgccagctccgtagggtcccagtcaagtgctggcagtgtggggagcagtggggtggctgccactatcagcaccccagtcaccatccagaatatgacatcttcctatgtcaccatcacatcccatgttcttaccgcctttgacctttgggaacaggccgaggccctcacgaggaagaataaagaattctttgctcggctcagcacaaatgtgtgcaccttggccctcaacagcagtttggtggacctggtgcactatacacgacagggttttcagcagctacaagaattaaccaaaacaccttaatggagccccaggttgattcaatgccttgggaactatttttgcacattggaagcctcaaaaacagtccagacgtttgtttcatcaggacaccaaactctaaaaaagaagcaccacgagatggccaggacatttgtccacttaaactctcaacaacagtgtgatcattggttggacactgtggttatgcagaagcagagatgaggaggctggccccagagatgatcttgcccttcctaactaaaggacagaagtgcaatttagcttaaatgggtgtatgaatggtctagaaacatttctattttttttttaaaccagcaggatacaagttgcaaatgaaatgaggagaaacagtttcaactctgaaagtgaatttcacgtcatctcagtagccacgctagtccattcccagaaggaaatttttttttttaacaatgacttttggtaaagggttttgtggatgattttttttcttttgagttttgggagaaatatttgtttaataacttctaatggccatctgtaaaccataagtaatgaaggactccactgtgccccactttctgccaatgaacagtggcttgataataccaagtattgttgtaatttataaaattgaaggcaacccccgctcctgccgcccccaatctccccattgcctagagcgctgcacattgaccccagctctgacttctcattactgtgctgaaagtcagcccacgtcggagcggtgaggaggagccacagcacatggggtgccacctcgaggtctgcacaggaggacttggcgctgccatttcctacccctgccatttcccacccctgcttcagcgaaagggactctctaacagggcagtcactgttgactctattctgaatttcctcccttggggaagaagggaaccaacatttatacctgaccagatggctaaagtgcttttaaagttttgtttaagtagagctggaatttgaggtgctgatctgtggtctacagttatgtggtaactcatgttgtccagccaactcagagtttcgtcagtgaacaagaaacatgaaatctgcttcttagagaggctatatttttctgctacaaatattttatatttatagcaaaactagactttcagagtccttgattgtctaggggaagttaactccctgagaggatgtagagatttggggtggttgattagacttttgaaaaactcatcaccacatgccttcactccagagtgttctcagctagatttgatttggttgaggaggaactgtggccctccgtaagttattgccatagtgtatgcattaaaccaagtccattttgaatgacctaaaatgaagtaacacaatcagaaatcccatgtgcccataagcacagatttttctttttcattgaaactttaaaggttattattggaaacattactttgagtgcagtgtttttaaaagccaattcttttttatcccttttagaagtagaatttgcacacttactacaattgaggagtgtcatctctataactttttctccgcctttgtcccattctgcccctggacatgtttcctaccaagcatgtttcacattttcctattagtggaggagggagaaccatatttatttataatgaagacatctaagatccctatgatgaatgcaggaactctcttggtagtttgtaaatacacaaagggatgtgtcgagggatgggagcgatgcttatctctcacagtgtgagtggtctgtgtgaggctgttccttcagttcttctccagactgttctttggttgtcacttaagtcagaggtctggtccctcatgtttaggtgaaagccagagaatgacagctgtagtcatatctgagcataagaccttgatgtgtgattcctgatgaccggtttcatttattcatgtaataaagcaaaggccctggtcctttttaaactactagttttaaaaacctgtgttaaatgaacagtaattgcctggtaggtttggtgtgtgtgtagcattgtgtgtccatctgttatatgtaaaggacaaggcaccagaatcaggctttatttcgatattgaagatgttatttaacatctttcttttttccttactcccttagccatcccctccccttttgtcctatcattccctagaacaagccacctgtcaattgtgaagggttgtgttctttatggcaggttctatgcagattgtgccagagcatgtgcgtgttctgttggcaagccacagtgctcccttgactgaagacatttccaggtagatttctcagccagctctaaaacagattgctttttcagtggccttactctttgtgggtttttttttttctctgaacttgatataaagattttatttgtcccttgaaaaagtaacaaatgtgcatagatcaatttgtactactttggtcattggatatttctgatccttattgcattgtacctaaaggagagtaactaatggtaacctttttaatagagtatgtgaaaggtagtggctgatgaatccttaacgttcatagggtctttttgctgttacggttgtatatagaggtctgaaggatttttaaaatgatttgcactttttcactgcatgcttacaattcccaaaggcaaaatctgtactgaggtagatcatttgaaagggctagattataaaattaagccttagagtatggaaagttcttataacaataatagtacacacttcagagtaagacaaatgcaaagcatcttaaggagtgaaaatagagtctaaatcttgcctttggcactacaaggtgtgtgtgtgtgtgtgtgttgtgtgtctttagtaggaaatggaagaacactgttttattttttaaagtgtttaatgtttctgtcctttctgtgaattattgaatttaagagccctgctaaataatgaaaaaacactttactaaaatttatcaaattatactgggttcggattgtgaaaacattggccacctagtagcagtggtgaggagtgggagggcccagcaagcatttatcagaaatagaatcacaataggaggagaatttggctgtctgatattatgatttgattacaatactgaatgggaaaagtatctaatattttgtaacaaaaagaccttcatattatctgttttgaccaaaatatgtagctatttcccttacacagattggaccgcacttatctcccttgtcctgtatcctttaatttcaggtctcaggatgtttagaaagctaaaaccccctacccctttctggctgaaaacttgccttatttggtatcttacacattaatgttactagcatcaggagcttactgttttattatgattcatcttcagtaatttttagaagcaagaagaaagccattgtgtcctctacaattaacaaaacttatctctgatatacaaagggatataaatatatacacttaaatagagaaaaagaggttgattgaattgtgcctttgagtgaacccagtttttaaataccgctgtgtttgtttcgccatggcttcagggatgctacatggctcttgcaccttttactcctctgctttatgaagtttgagttgtatttgtgcatcttaaagtaggttgaggcttgaggctgggctttcgggtttttttgttttttgttttgttttgttttgttttgttttcttgtacttaaacctgcttgcttcctaccacagattctttattttcccaaacactacaaaaaaacttttaaaactttgccatttcatctgtttacactctttgccactgattagcagtatttaaatcttgcaagaatattttgtgctttctttagaaacacaagagtatagatttttctcactgaaaagtgagagttacgcattgcagccatgaagggatgctaggatcaattatggcagtaccttttttcccctcctgttcttgagccagttgtctcttttgttttgggtcccacttaggattaacggatgtaaggtattttcctgtgcctttattttgtgtcattctattggaaggaggtgtaacggcagaatagcatcgtgttgggggttttccttcaaacactgcaagtgatattgccaccatgtgaacctcaaatatgcaatccagttgtgttggtttctcggtgacttggagtgttcatctcttcatgaattgtgagcactgaccatgttcttcagttcttaattatggtgagttgacaaataccaactactgcttttctttaggtggctataaatttcttactgtcaggaggaaatgacattatattctgttccactgaacgtcagagatcagcaggcactgtactgggtagagaagtgcctatacttctctacctaagagggcaggagggaaaccctacagctccttgtgagcctatatattagtatatcggcctggagaggacaagggaataagaccactcatagtgaggctggccaagctgcactggtcggaccaggcagtggctgacctaaggaaggcaacttgctttgcttaaaagtagattttttaagcaatgcttaacacaggcagcattcacctttgttcaggccatcgacatgtattgttaaaattactgcatatccccctcagatatcaagtatacactgttcatgttggggttgtgtgtgtgtatgtgtgtatgtacgcacgcatgtgtcccaaatcttgttttaattttttttttctgaatgtgatcatgttttggataatacctgagcagggttgccttttttttatttattaccattatatattatattatattatatattttttgctttcttataactttggaggaaagtcaaatcttggtattattaaaattgttttaaaaaggagtaaattttccagttgataaatgaaaatcactggcctatgtttaataagtttttctttaattactgtggaataacgtgccagctatcatcaacacaatgattttgtacatagggtagggaagcagtgatgctctcaatgggaagatgtgcaacacaaattaaggggaactccatgtattttacctacttcagcaatggaactgcaacttggggctttgtgaataaaatttagctgccttgtatagtcgtttgaaagaatatgtgatctgtgagagaattatagtttttttttagaagaaaaatctgcaaaagatctttccaaagacaatgtgccacagatcttttgttctctgtaatgaggattaattgctgtttaaacaaaaatgtaattgttcatctttaaattctttccttttcataagaggatcaagctgtaaaaaaacaaaaaaattaataaaaatttcgagaaatcaaaaaaaaaaa

SEQ ID NO: 92 - DNA, 3842 bp, Homo sapiens
gccctcttgattatgtgtgccctctccgggcgcccgcgttagcggccgggtggaggtggggagggaagacgctgaggaggaggaggaggcggaggaggcggtggaggggaggtggggggaatcagcaaggacatggctcctgactcctgtgcggaacgtgagtgactgagcggcaaagcccgaatggtctctagcgaccggcccgtgtcactggaggacgaggtctcccatagtatgaaggagatgattggaggctgttgcgtttgctcagacgagagaggctgggccgagaacccgctggtttattgcgacgggcacggctgcagcgtcgcggtgcatcaagcttgctatggcattgttcaagtacccactggaccgtggttttgcaggaaatgtgaatctcaggagagagcagccagagtgagatgtgaactttgtccccataaggatggagctttaaaaagaacagataatgggggttgggcccatgtggtttgtgccctgtatattccagaggtacaatttgccaatgtttccacaatggaaccaattgttttacagtctgttccgcatgatcgttataataagacttgctacatttgtgatgaacaaggaagagaaagcaaagcagccactggtgcttgcatgacatgtaataaacatggatgtcgacaggctttccatgtaacatgcgctcagtttgccggactgctttgtgaagaagaaggtaatggtgccgataatgtccaatactgtggctactgtaaataccattttagtaagctgaaaaagagcaaacggggatctaataggtcatatgatcaaagtttaagtgattcttcctctcactctcaggataaacatcatgagaaagagaaaaaaaaatataaagagaaggacaaacacaaacagaaacacaagaagcagccagaaccatcacctgcattggttccatccttgactgttactacagaaaaaacttatacaagcactagcaacaactctatatctggatcattgaagcgcttggaagatactactgcacgatttacaaatgcaaatttccaggaagtctctgcacacacctctagtggaaaagatgtttcagagactagagggtcagagggcaaagggaagaaatcttcagctcacagctcaggtcaaaggggaagaaagcctggtggtggaagaaatccaggaacaactgtgtcagcagctagcccttttcctcaaggcagtttttcaggaactccaggcagtgtaaagtcatcttctggaagttcagtgcagtctccccaggatttcctgagctttacagactcagatctgcgtaatgacagttactctcactcccaacagtcatcagcaaccaaagatgtacataaaggagagtctggaagccaggaagggggggtaaatagttttagtaccttaattggcctcccttcaacctcagctgttacttcacagcctaaaagctttgaaaattcacctggagatttgggtaattccagccttcctacagcaggatataagcgggctcaaacttctggcatagaagaagaaactgtaaaggaaaagaaaaggaaaggaaataaacaaagtaagcatgggcctggcagacccaaaggaaacaaaaatcaagagaatgtttctcatctctcagtttcttctgcttcaccaacatcatctgtagcatcagctgcaggaagcataacaagctctagtctgcagaaatctcctacattgctcaggaatggaagtttacagagcctcagtgttggctcatctccagttggttcagaaatttccatgcagtatcggcatgatggagcttgcccaacaactacgttctcagagttgctgaatgcaatacacaacgacagaggtgacagttctacactaacaaagcaagaacttaaattcataggtatttataacagcaatgatgtagcagtatcgtttccaaatgtagtatctggctcgggatctagtactcctgtctccagctctcacttacctcagcagtcttctgggcatttgcaacaagtaggagcgctctctccctcagctgtgtcatctgcagcccctgctgttgctacaactcaggcaaatactctatctggatcttctctcagtcaggcaccatctcatatgtatggcaatagatcaaattcatcaatggcagctcttatagctcagtctgaaaacaatcaaacagatcaagatcttggagacaatagccgcaacctagttggcagaggaagctcaccccgaggaagtctctcgccacgatcccctgtaagcagcttacagattcgctatgatcaaccaggcaacagcagtttggaaaatctgcctccagtagcagccagcatagaacagcttttggagaggcagtggagtgaaggacagcaatttttactagaacagggtactcctagtgacattttaggaatgctgaagtcattacaccaacttcaagttgaaaaccgaagattagaggaacaaattaaaaacttgactgccaaaaaggaacggcttcagttattgaatgcacagctttcagtgccttttccaacaataacagcaaatcctagtccgtctcatcaaatacacacattttcagcacagactgctcctactactgattccttgaacagcagtaagagccctcatataggaaacagctttttacctgataattctcttcctgtattaaatcaggacttaacctccagtggacaaagtaccagcagctcatcagctctttctaccccacctcctgctgggcagagtccggctcaacaaggctcaggagtgagtggagttcagcaggtcaatggcgtgacagtgggggcactagctagtggaatgcagcctgtaacttccaccattcctgccgtgtctgcagtgggtggaataattggagctttgccaggtaaccaactggcaattaatggcattgtaggagctttaaatggggttatgcagactcctgtcacaatgtcccagaaccctacccctctcacccacacaaccgtaccacctaatgcaacacatccaatgccagctacactgactaacagtgcctcaggactaggattactttctgaccagcaacgacaaatacttattcatcaacagcagtttcagcagttgttaaattctcaacagctcacaccagtacacaggcacccccacttcacacagctaccaccaacccatttctcaccatccatggagataatgcaagtcagaaagtagcaagacttagtgataaaactgggcctgtagctcaagagaaaagttgacacctgagaaacatctagaaattgcctatcctgctgttctagcacttcatctggctgcctttgcagtccttttactacagctatgaagaaacgcaacaagaaactcaatgcacaacaaaggattaattgctgcaaggacattcttgtaaggctttgattagttttcttgttgctttgttgcactgaaatggaattcccatgcccctaccccttaccccagttttttgaacatggaaagaaaatttaataactttttaaagtgacataatttacatgcaatatgtttatcaactcaagaatttaatatagttggtacacaactagttttgtttataaattggagatgcaaatagcaaaactaaatacttgctccatttacaaactacttgattttattgtacaagttgaaatatgctcttttgtttgggttacagtatgcttgctctaagtcaaattccaaggaactaatttcttctcctggagttgcattgattcagtattacaaatatatagcacatcacctgggac

SEQ ID NO: 93 - DNA, 15750 bp, Homo sapiens
atggacagccagaacctggctggtgaggataaagattcacaaccggcagctgatggacctgcagcctctgaggacccaagtgccactgagtcagacctgcccaacccacatgtgggagaggtctctgtccttagttctgggagtcccaggcttcaggagactcctcaggactgcagtgggggtccggtgcggcgttgtgctctctgtaactgcggggagcccgctctacacgggcagcgggagctacggcgctttgagttgccatttgattggccccggtgtccagtggtgtcccctggggggagcccagggcccaatgaggcagtgctgcccagtgaggacctatcacagattggtttccctgagggccttacacctgcccacctaggagaacctggagggtcctgctgggctcaccattggtgtgctgcatggtcggcaggcgtatgggggcaggagggcccacaactatgtggtgtggacaaggccatcttctcagggatctcacagcgctgctcccactgcaccaggctcggtgcctccatcccttgccgctcacctggatgtccacggctttaccacttcccctgcgcgactgccagcggttccttcctatccatgaaaacactgcagctgctatgcccagagcacagtgagggggctgcatatctggaggaggctcgctgtgcagtgtgtgaggggccaggggagctgtgtgacctgttcttctgtaccagctgtgggcatcactatcacggggcctgcctggacactgctctgactgcccgcaaacgtgctggctggcagtgccctgaatgcaaagtgtgccaagcctgcaggaaacctgggaatgactctaagatgttggtttgcgagacgtgtgacaaaggataccatactttctgcctaaaaccacccatggaggaactgcctgctcactcttggaagtgcaaggcgtgccgggtgtgccgggcctgtggggcgggctcagcagaactgaatcccaactcggagtggtttgagaactactctctctgtcaccgctgtcacaaagcccagggaggtcagactatccgctccgttgctgagcagcataccccggtgtgtagcagattttcacccccagagcctggcgatacccccactgacgagcccgatgctctgtacgttgcatgccaagggcagccaaagggtgggcacgtgacctctatgcaacccaaggaaccagggcccctgcaatgtgaagccaaaccactagggaaagcaggggtccaacttgagccccagttggaggcccccctaaacgaggagatgccactgctgcccccacctgaggagtcacccctgtccccaccacctgaggaatcacccacgtccccaccacctgaggcatcacgcctgtcgccaccacctgaggaattgcccgcatccccacttcctgaggcattgcacctgtcccggccgctggaggaatcgcccctctctccgccgcctgaggagtctcctctgtctcccccacctgaatcatcacctttttctccactggaggagtcgcccttgtctccaccggaagagtcacccccatctcctgcacttgagacgcctctatccccaccacctgaagcatcgcccctgtccccaccatttgaagaatctcctttgtccccgccacctgaggaattgcccacttccccgccacctgaagcatctcgcctgtctccaccacctgaggagtcacccatgtcccctccacctgaagagtcacccatgtctccaccaccggaggcatctcgtctgttcccaccatttgaagagtctcctctgtcccctccacctgaggagtctcccctttccccaccacctgaggcatcacgcctgtccccaccacctgaggactcgcctatgtccccaccacctgaagaatcacctatgtcccccccacctgaggtatcgcgcctatcccccctgcctgtggtgtcacgcctgtctccaccgcctgaggaatctcccttgtccccaccggccctgtctcctttgggggagttagagtacccctttggtgccaaaggggacagtgaccctgagtcaccgttggctgcccccatcctggagacacccatcagccctccaccagaagctaactgcactgaccctgagcctgtcccccctatgatccttcccccatctccaggctccccagtggggccggcttctcccatcctgatggagccccttcctcctcagtgttcgccactccttcagcattccctggttccccaaaactcccctccttcccagtgctctcctcctgccctaccactgtccgttccctccccgttgagtcccatagggaaggtagtgggggtctcagatgaggctgagctgcacgagatggagactgagaaagtttcagaacctgaatgcccagccttggaacccagtgccaccagtcctctcccttccccaatgggggacctttcctgccccgcccccagccctgccccagccctggatgacttctctggcctaggggaagacacagcccctctggatgggattgatgctccgggttcacagccagagcctggacagacccctggcagtttggctagtgaacttaaaggctcccctgtgctcctggaccccgaggagctggcccctgtgacccctatggaggtctaccccgaatgcaagcagacagcagggcggggctcaccatgtgaagaacaggaagagccacgtgcaccggtggcccccacaccacccactctcatcaaatccgacatcgttaacgagatctctaatctgagccagggtgatgccagtgccagttttcctggctcagagcccctcctgggctctccagacccggaggggggtggctccctgtccatggagttgggggtctctacggatgttagtccagcccgagatgagggctccctacggctctgtactgactcactgccagagactgatgactcactattgtgcgatgctgggacagctatcagcggaggcaaagctgagggggagaaggggcggcggcgcagctccccagcccgttcccgcatcaaacagggtcgcagcagcagtttcccaggaagacgccggcctcgtggaggagcccatggagggcgtggtagaggacgggcccggctaaagtcaactgcttcttccattgagactctggtagttgctgacattgatagctctcccagtaaggaggaggaggaagaagatgatgacaccatgcagaataccgtggttctcttctccaacacagacaaatttgtcctaatgcaggacatgtgtgtggtatgtggcagctttggccggggggcagagggccacctccttgcctgttcgcagtgctctcagtgctatcacccttactgtgtcaacagcaagatcaccaaggtgatgctgctcaagggctggcgttgtgtggagtgtattgtgtgtgaggtgtgtggccaggcctccgacccctcacgcctgctgctctgtgatgactgtgatattagctaccacacatactgcctggaccccccactgctcaccgtccccaagggcggctggaagtgcaagtggtgtgtgtcctgtatgcagtgtggggctgcttcccctggcttccactgtgaatggcagaatagttacacacactgtgggccctgtgccagcctggtgacctgccctatctgtcatgctccttacgtagaagaggacctactaatccagtgccgccactgtgaacggtggatgcatgcaggctgtgagagcctcttcacagaggacgatgtggaccacgcacccgatgaaggctttgactgtgtctcctgccagccctacgtggtaaagcctgtggcgcctgttgcacctccagagctggtgcccatgaaggtgaaagagccagagccccagtactttcgcttcgaaggcgtgtggctgacagaaactggcatggccttgctgcgtaacctgaccatgtcaccactgcacaagcggcgccaacggcgaggacggcttggcctcccaggcgaggcaggattggagggttctgagccctcagatgcccttggccctgatgacaagaaggatggggacctggacaccgatgagctgctcaagggtgaaggtggtgtggagcacatggagtgcgaaattaaactggagggccccgtcagccctgatgtggagcctggcaaagaggagaccgaggaaagcaaaaaacgcaagcgtaaaccatatcggcctggcattggtggtttcatggtgcgacagcggaaatcccacacacgcacgaaaaaggggcctgctgcacaggcggaggtgttgagtggggatgggcagcccgacgaggtgatacctgctgacctgcctgcagagggcgccgtggagcagagcttagctgaaggggatgagaagaagaagcaacagcggcgagggcgcaagaggagcaaactggagggcatgttccctgcttacttgcaggaagccttctttgggaaggagctgctggacctgagccgtaaggccctttttgcagttggggtgggccggccaagctttggactagggaccccaaaagccaagggagatggaggctcagaaaggaaggaactccccacatcgcagaaaggagatgatggtccagatattgcagatgaagaatcccgtggcctcgagggcaaagccgatacaccaggacctgaggatgggggcgtgaaggcatccccagtgcccagtgaccctgagaagccaggcaccccaggtgaagggatgcttagctctgacttagacaggatttccacagaagaactgcccaagatggaatccaaggacctgcagcagctcttcaaggatgttctgggctctgaacgagaacagcatctgggttgtggaacccctggcctagaaggcagccgtacgccactgcagaggccctttcttcaaggtggactccctttgggcaatctgccctccagcagcccaatggactcctacccaggcctctgccagtccccgttcctggattctagggagcgcgggggcttctttagcccggaacccggtgagcccgacagcccctggacgggctcaggtggcaccacgccctccacccccacaacccccaccacggagggtgagggcgacggactctcctataaccagcggagtcttcagcgctgggagaaggatgaggagttgggccagctgtccaccatctcgcctgtgctctatgccaacattaattttcctaatctcaagcaagactacccagactggtcaagccgttgcaaacaaatcatgaagctctggagaaaggttccagcagctgacaaagccccctacctgcaaaaggccaaagataaccgggcagctcaccgcatcaacaaggtgcagaagcaggctgagagccagatcaacaagcagaccaaggtgggcgacatagcccgtaagactgaccgaccggccctacatctccgcattcccccgcagccaggggcactgggcagcccgccccccgctgctgcccccaccattttcattggcagccccactacccccgccggcttgtctacctctgcggacgggttcctgaagccgccggcgggctcggtgcctggccctgactcgcctggtgagctcttcctcaagctcccaccccaggtgcccgcccaagcgccttcgcaggacccctttggactggcccctgcctatcccctggagccccgcttccccacggcaccgcccacctatcccccctatcctagtcctacgggggcccctgcgcagcccccgatgctgggcgcctcatctcgtcctggggctggccagccaggggaattccacactaccccacctggcacccccagacaccagccctccacacctgacccgttcctcaaaccccgctgcccctcgctggataacttggctgtgcctgagagccctggggtagggggaggcaaagcttccgagcccctgctctcgcccccaccttttggggagtcccggaaggccctagaggtgaagaaggaagagcttggggcatcctctcctagctatgggcccccaaacctgggctttgttgactcaccctcctcaggcacccacctgggtggcctggagttaaagacacctgatgtcttcaaagcccccctgacccctcgggcatctcaggtagagccccagagcccgggcttgggcctaaggccccaggagccaccccctgcccaggctttggcaccttctcctccaagtcacccagacatctttcgccctggctcctacactgacccatatgctcagcccccattgactcctcggccccaacctccgccccctgagagctgctgtgctctgccccctcgctcactgccctccgaccctttctcccgagtgcctgtcagtcctcagtcccagtccagctcccagtctccactgacaccccggcctctgtctgctgaagctttttgcccatcacccgttacccctcgcttccagtcccctgacccttattctcgcccaccctcacgccctcagtcccgtgacccatttgccccattgcataagccaccccgaccccagccccctgaagttgcctttaaggctgggtctctagcccacacttcgctgggggctggggggttcccagcagccctgcccgcggggccagcaggtgagctccatgccaaggtcccaagtgggcagccccccaattttgtccggtcccctgggacgggtgcatttgtgggcaccccctctcccatgcgtttcactttccctcaggcagtaggggagccttccctaaagccccctgtccctcagcctggtctcccgccaccccatgggatcaacagccattttgggcccggccccaccttgggcaagcctcaaagcacaaactacacagtagccacagggaacttccacccatcgggcagccccctggggcccagcagcgggtccacaggggagagctatgggctgtccccactacgccctccgtcggttctgccaccacctgcacccgacggatccctcccctacctgtcccatggagcctcacagcgatcaggcatcacctctcctgtcgaaaagcgagaagacccagggactggaatgggtagctctttggcgacagctgaactcccaggtacccaggacccaggcatgtccggccttagccaaacagagctggagaagcaacggcagcgccagcggctacgagagctgctgattcggcagcagatccagcgcaacaccctgcggcaggagaaggaaacagctgcagcagctgcgggagcagtggggcctccaggcagctggggtgctgagcccagcagccctgcctttgagcagctgagtcgaggccagaccccctttgctgggacacaggacaagagcagccttgtggggttgcccccaagcaagctgagtggccccatcctggggccagggtccttccctagcgatgaccgactctcccggccacctccaccagccacgccttcctctatggatgtgaacagccggcaactggtaggaggctcccaagctttctatcagcgagcaccctatcctgggtccctgcccttacagcagcaacagcaacaactgtggcagcaacaacaggcaacagcagcaacctccatgcgatttgccatgtcagctcgctttccatcaactcctggacctgaacttggccgccaagccctaggttccccgttggcgggaatttccacccgtctgccaggccctggtgagccagtgcctggtccagctggtcctgcccagttcattgagctgcggcacaatgtacagaaaggactgggacctgggggcactccgtttcctggtcagggcccacctcagagaccccgtttttaccctgtaagtgaggacccccaccgactggctcctgaagggcttcggggcctggcggtatcaggtcttcccccacagaaaccctcagccccaccggcccctgaattgaacaacagtcttcatccaacaccccacaccaagggtcctaccctgccaactggtttggagctggtcaaccggcccccgtcgagcactgagcttggccgccccaatcctctggccctggaagctgggaagttgccctgtgaggatcccgagctggatgacgattttgatgcccacaaggccctagaggatgatgaagagcttgctcacctgggtctgggtgtggatgtggccaagggtgatgatgaacttggcaccttagaaaacctggagaccaatgacccccacttggatgacctgctcaatggagacgagtttgacctgctggcatatactgatcctgagctggacactggggacaagaaggatatcttcaatgagcacctgaggctggtagaatcggctaatgaggaggctgaacgggaggccctgctgcggggggtggagccaggacccttgggccctgaggagcgccctccccctgctgctgatgcctctgaaccccgcctggcatctgtgctccctgaggtgaagcccaaggtggaggagggtggacgccacccttctccttgccaattcaccattgctacccccaaggtagagcccgcacctgctgccaattcccttggcctggggctaaagccaggacagagcatgatgggcagccgggatacccggatgggcacagggccattttctagcagtgggcacacagctgagaaggcctcctttggggccacgggagggccaccagctcacctgctgacccccagcccactgagtggcccaggaggatcctccctgctggaaaagtttgagctcgagagtggggctttgaccttgcctggtggacctgcagcatctggggatgagctagacaagatggagagctcactggtagccagcgagttacccctgctcattgaggacctgttggagcatgagaagaaggagctgcagaagaagcagcagctttcagcacagttgcagcctgcccagcagcagcagcaacagcagcagcagcattccctactgcctgcaccaggccctgcccaggccatgtctttgccacatgagggctcttctcccagtttggctgggtcccaacagcagctttccctgggtcttgcagttgcccgacagccaggtttgccccagccactgatgcccacccagccaccagctcatgccctccagcaacgcctggctccatccatggctatggtgtccaatcaagggcatatgctaagtgggcagcatggagggcaggcaggcttggtaccccagcagagctcacagccagtgctatcacagaagcccatgggcaccatgccaccttccatgtgcatgaagccgcagcaattggcaatgcagcagcagctggcaaacagcttcttcccagatacagacctggacaaatttgctgcagaagatatcattggtcccattgcaaaggccaagatggtggctttgaaaggcatcaagaaagtgatggctcagggcagcattggggtggcacctggtatgaacagacagcaagtgtctctgctagcccagaggctctcggggggacctagcagtgatctgcagaaccatgtggcagctgggagtggccaggagcggagtgctggtgatccctcccagcctcgtcccaacccgcccacttttgctcagggagtgatcaatgaagctgaccagcggcagtatgaggagtggctgttccatacccagcagctcctacagatgcagctgaaggtgctagaggagcagattggtgtacaccgcaagtcccggaaggctctgtgtgccaagcagcgcactgccaaaaaagctggccgtgagttcccagaagctgatgctgagaagctcaagctggttacagagcagcagagcaagatccagaaacaactggatcaggtccggaaacagcagaaggagcacactaatctcatggcagaatatcggaacaagcagcagcaacaacagcagcagcagcagcaacaacagcaacagcactcagctgtgctggctctcagcccttcccagagtccccggctgctcaccaagctccctggtcagctgctccctggccatgggctgcagccaccacaggggcctccgggtgggcaagccggaggtcttcgcctgacccctgggggtatggcactacctggacagcctggtggccccttccttaatacagctctggcccaacagcagcaacagcaacattctggtggggctggatccctggctggcccttcagggggcttcttccctggcaaccttgctcttcgaagcctcggacctgattcaaggcttttacaggaaaggcagctgcagctgcagcagcaacgtatgcagctggcccagaaactgcagcagcagcagcagcagcaacagcagcagcagcaccttctaggacaggtggcaatccagcagcaacagcagcagggtcctggagtacagacaaaccaagctctgggtcccaagccccagggccttatgcctcccagcagccaccaaggcctcctggtccagcagctgtcccctcaaccaccccaggggccccagggcatgctgggccctgcccaggtggctgtgttgcagcagcagcaccctggagctttgggcccccagggccctcacagacaggtgcttatgacccagtcccgggtgctcagttccccccagctggcacagcagggtcagggccttatgggacacaggctggtcacagcccagcagcagcagcagcaacaacagcaccaacagcaagggtccatggcagggctgtcccatcttcagcaaagtctgatgtcacacagtgggcagcccaaactgagcgctcagcccatgggctctttacagcagcttcagcagcagcagcagctgcaacagcaacagcaacttcagcagcagcagcagcagcagctacaacagcaacagcaacttcagcagcaacagcttcaacagcagcaacagcagcagcagcttcaacaacagcagcagcaacagcttcaacagcagcaacagcagctacaacagcaacagcaacaacaacagcagcagtttcaacagcagcagcaacagcagcagatgggccttttaaaccagagtcgaactttactgtcccctcagcaacaacagcagcagcaagtggcacttggccctggcatgccagcaaagcctcttcaacacttttctagccctggagccctgggtccaaccctcctcctgacgggcaaggaacaaaacaccgtagacccagccgtttcttcagaggccactgaggggccctctacacatcagggagggccgttagcaataggaactacccctgagtcaatggccactgaaccaggagaggtaaagccctcactctctggggactcacaactcctgcttgtccaaccccagccccagcctcagcccagctctctgcagctgcagccacctctgaggcttccaggacaacagcagcagcaagttagcctgctccacacagcaggtggaggaagccatgggcagctaggcagtggatcatcttctgaggcctcatctgtgccccacctgctggctcagccctctgtttccttaggggatcagcctgggtccatgacccagaaccttctgggcccccaacagcccatgctagagcggcccatgcaaaataatacagggccacaacctcccaaaccaggacctgtcctccagtctgggcagggtctgcctggggttggaatcatgcctacggtgggtcagcttcgagcacagctccaaggagtcctggccaaaaacccacagctgcggcacttaagtcctcagcagcagcagcagctacaggcactcctcatgcagcggcagctgcagcagagtcaggcagtacgccagaccccaccctaccaggagcctgggacccagacctctcccctccagggcctcctgggctgccaacctcaacttgggggcttccctggaccacagacaggccccctccaggagctaggggcagggcctcgacctcagggcccaccccggctccctgccccaccaggagccttatctacaggaccagtccttggccctgtccatcccacacctccaccatccagccctcaagagccaaagagaccttcacaattaccttcccccagctcccagcttcccactgaggcccagctccctcccacccatccagggacccccaaacctcaggggccaaccttggagccgcctcctgggagggtctcacctgctgctgcccagcttgcagataccttgtttagcaagggtctgggaccttgggatcccccagacaacctagcagaaacccagaagccagagcagagcagcctggtacctgggcatctggaccaggtgaatggacaggtggtgcctgaggcatcccaactcagcatcaagcaggaacctcgggaagagccatgtgccctgggagcccagtcagtgaagagggaggccaatggggagccaataggggcaccaggaaccagcaaccacctcctgctggcaggccctcgctcagaagctgggcatctgctcttgcagaagctactccgggcaaagaatgtgcaactcagcactgggcaggggtccgaggggctgcgagctgagatcaacgggcacattgacagcaagctggctgggctggagcagaaactacagggtacccccagcaacaaggaggatgcagcagcaaggaagcctttgacaccgaagcccaagcgggtacagaaggcaagcgacaggttggtgagctcccgaaagaagctgcggaaggaggacggcgtcagggccagcgaggccttgctgaaacagctgaaacaggagctgtccctgctgcccctaacggagcctgctatcaccgccaattttagcctctttgccccctttggcagtggctgcccagtcaatgggcagagccagctgaggggggcctttggaagtggggcgctgcccactggccctgactactattcccagctgcttaccaagaataacctgagtaacccgccgacaccaccctcgtcgctgccccccaccccacccccatcggtgcagcagaagatggtgaatggcgtcaccccatctgaagagctgggggagcaccccaaggatgctgcctctgcccgggatagtgaaagggcactgagggatacttcagaggtgaagagtctagacctgctggctgccttgcctacaccccctcacaatcagactgaggatgtcaggatggagagtgatgaggatagcgattctcctgacagcattgtgccagcttcatcccctgagagcatcttgggggaggaggcccctcgtttccctcatctgggctcaggccggtgggagcaagaggaccgggccctctcccctgtcatccccctcattcctcgggacagcatcccagtcttcccagataccaaaccttatggggcccttggcctggaggtccctggaaagctgcctgtcacaacttgggaaaagggcaaaggaagtgaggtgtcagtcatgctcacagtctctgctgctgcagacaagaacctgaatggcgtgatggtggcagtggcggagctgctgagcatgaagatccccaactcctatgaggtgctgttcccagagagccccgcccggggaggcactgagccaaagaagggggaagctgagggtcctggtgggaaggaaaagggtctggaaggcaagagcccagacactggccctgattggctgaagcagtttgatgcagtgttggctggctataccctgaagaggcaactagacatcttgagcctcctgaaacaggagagccccgccccagagccacccactcagcacaggtatacctacaatgtctccaatctggatgtgcgacagctctcggccccacctcctgaagaaccctccccgcccccttcccccttggcaccttctcctgccagtccccctactgagcccttggttgaacttcccaccgaacccttggctgagccacccgtcccctcacctctgccactggcctcatcccctgaatcagcccgacccaagccccgtgcccggccccctgaagaaggtgaagatacccgtcctcctcgcctcaagaaatggaaaggagtgcgctggaagcggcttcggctgctgctgaccatccagaagggcagtggacggcaggaggatgagcgggaagtggcagagtttatggagcagcttggcacagccttgcgacctgacaaggtaccgcgagacatgcgtcgctgctgtttctgtcatgaggagggtgacggggccactgatgggcctgcccgtctgctgaacctggacctggacctgtgggtgcacctcaactgtgccctttggtccacggaggtgtatgagacccagggcggagcactgatgaatgtggaggttgccctgcaccgaggactgctaaccaagtgctccctgtgccagcgaactggtgccaccagcagctgcaatcgcatgcgttgccccaatgtctaccattttggttgtgccatccgcgccaagtgcatgttcttcaaggacaagaccatgctgtgtccaatgcataagatcaaggggccctgtgagcaagagctgagctcttttgctgtcttccggcgggtctacattgagcgggacgaggtgaagcaaatcgctagcatcattcagcggggagaacggctgcacatgttccgtgtgggggggcttgtgttccacgccatcggacagctgctgcctcaccagatggctgactttcatagtgccactgccctctatcccgtgggctacgaggccacgcgcatctattggagcctccgcaccaacaatcgtcgctgctgctatcgctgttctattggtgagaacaacgggcggccggagtttgtaatcaaagtcatcgagcagggcctggaggacctggtcttcactgacgcctctccccaggccgtgtggaatcgcatcattgagcctgtggctgccatgagaaaagaggctgacatgctgcgactcttccctgagtatctgaagggcgaggagctctttgggctgacggtgcatgccgtgcttcgcatagctgaatcactgcccggggtggagagctgtcaaaactatttattccgctatgggcgccacccccttatggagctgccactcatgatcaaccccactggctgtgcccgatcagagcctaaaatcctcacacactacaaacggccccataccctgaacagcaccagcatgtctaaggcatatcagagcaccttcacaggcgagaccaacaccccctacagcaagcagtttgtgcactccaagtcatctcagtaccggcggctgcgcaccgaatggaagaacaacgtgtacctggctcgctcccgtatccagggcctggggctctatgcagccaaggacctagaaaagcacacaatggttatcgagtacattggcaccatcattcggaacgaggtggccaaccggcgggagaaaatctacgaagagcagaatcgaggcatctacatgttccgaataaacaatgaacatgtgattgatgctacgttgaccggcggccctgccaggtacattaaccattcctgtgcccctaactgtgtggccgaagtcgtgacatttgacaaagaggacaaaatcatcatcatctccagccggcgaatccccaaaggagaggagctaacctatgactatcagtttgattttgaggacgatcagcacgagatcccctgc

SEQ ID NO: 94 - DNA, 2091 bp, Homo sapiens
gagcggagccgcgggcgggagggcggacggaccgactgacggtagggacgggaggcgagcaagatggcgcagacgcagggcacccggaggaaagtctgttactactacgacggggatgttggaaattactattatggacaaggccacccaatgaagcctcaccgaatccgcatgactcataatttgctgctcaactatggtctctaccgaaaaatggaaatctatcgccctcacaaagccaatgctgaggagatgaccaagtaccacagcgatgactacattaaattcttgcgctccatccgtccagataacatgtcggagtacagcaagcagatgcagagattcaacgttggtgaggactgtccagtattcgatggcctgtttgagttctgtcagttgtctactggtggttctgtggcaagtgctgtgaaacttaataagcagcagacggacatcgctgtgaattgggctgggggcctgcaccatgcaaagaagtccgaggcatctggcttctgttacgtcaatgatatcgtcttggccatcctggaactgctaaagtatcaccagagggtgctgtacattgacattgatattcaccatggtgacggcgtggaagaggccttctacaccacggaccgggtcatgactgtgtcctttcataagtatggagagtacttcccaggaactggggacctacgggatatcggggctggcaaaggcaagtattatgctgttaactacccgctccgagacgggattgatgacgagtcctatgaggccattttcaagccggtcatgtccaaagtaatggagatgttccagcctagtgcggtggtcttacagtgtggctcagactccctatctggggatcggttaggttgcttcaatctaactatcaaaggacacgccaagtgtgtggaatttgtcaagagctttaacctgcctatgctgatgctgggaggcggtggttacaccattcgtaacgttgcccggtgctggacatatgagacagctgtggccctggatacggagatccctaatgagcttccatacaatgactactttgaatactttggaccagatttcaagctccacatcagtccttccaatatgactaaccagaacacgaatgagtacctggagaagatcaaacagcgactgtttgagaaccttagaatgctgccgcacgcacctggggtccaaatgcaggcgattcctgaggacgccatccctgaggagagtggcgatgaggacgaagacgaccctgacaagcgcatctcgatctgctcctctgacaaacgaattgcctgtgaggaagagttctccgattctgaagaggagggagaggggggccgcaagaactcttccaacttcaaaaaagccaagagagtcaaaacagaggatgaaaaagagaaagacccagaggagaagaaagaagtcaccgaagaggagaaaaccaaggaggagaagccagaagccaaaggggtcaaggaggaggtcaagttggcctgaatggacctctccagctctggcttcctgctgagtccctcacgtttcttccccaacccctcagattttatattttctatttctctgtgtatttatataaaaatttattaaatataaatatccccagggacagaaaccaaggccccgagctcagggcagctgtgctgggtgagctcttccaggagccaccttgccacccattcttcccgttcttaactttgaaccataaagggtgccaggtctgggtgaaagggatacttttatgcaaccataagacaaactcctgaaatgccaagtgcctgcttagtagctttggaaaggtgcccttattgaacattctagaaggggtggctgggtcttcaaggatctcctgtttttttcaggctcctaaagtaacatcagccatttttagattggttctgttttcgtaccttcccactggcctcaagtgagccaagaaacactgcctgccctctgtctgtcttctcctaattctgcaggtggaggttgctagtctagtttcctttttgagatactattttcatttttgtgagcctctttgtaataaaatggtacatttct

SEQ ID NO: 95 - DNA, 1985 bp, Homo sapiens
cgccgagctttcggcacctctgccgggtggtaccgagccttcccggcgccccctcctctcctcccaccggcctgcccttccccgcgggactatcgcccccacgtttccctcagcccttttctctcccggccgagccgcggcggcagcagcagcagcagcagcagcaggaggaggagcccggtggcggcggtggccggggagcccatggcgtacagtcaaggaggcggcaaaaaaaaagtctgctactactacgacggtgatattggaaattattattatggacagggtcatcccatgaagcctcatagaatccgcatgacccataacttgctgttaaattatggcttatacagaaaaatggaaatatataggccccataaagccactgccgaagaaatgacaaaatatcacagtgatgagtatatcaaatttctacggtcaataagaccagataacatgtctgagtatagtaagcagatgcatatatttaatgttggagaagattgtccagcgtttgatggactctttgagttttgtcagctctcaactggcggttcagttgctggagctgtgaagttaaaccgacaacagactgatatggctgttaattgggctggaggattacatcatgctaagaaatacgaagcatcaggattctgttacgttaatgatattgtgcttgccatccttgaattactaaagtatcatcagagagtcttatatattgatatagatattcatcatggtgatggtgttgaagaagctttttatacaacagatcgtgtaatgacggtatcattccataaatatggggaatactttcctggcacaggagacttgagggatattggtgctggaaaaggcaaatactatgctgtcaattttccaatgtgtgatggtatagatgatgagtcatatgggcagatatttaagcctattatctcaaaggtgatggagatgtatcaacctagtgctgtggtattacagtgtggtgcagactcattatctggtgatagactgggttgtttcaatctaacagtcaaaggtcatgctaaatgtgtagaagttgtaaaaacttttaacttaccattactgatgcttggaggaggtggctacacaatccgtaatgttgctcgatgttggacatatgagactgcagttgcccttgattgtgagattcccaatgagttgccatataatgattactttgagtattttggaccagacttcaaactgcatattagtccttcaaacatgacaaaccagaacactccagaatatatggaaaagataaaacagcgtttgtttgaaaatttgcgcatgttacctcatgcacctggtgtccagatgcaagctattccagaagatgctgttcatgaagacagtggagatgaagatggagaagatccagacaagagaatttctattcgagcatcagacaagcggatagcttgtgatgaagaattctcagattctgaggatgaaggagaaggaggtcgaagaaatgtggctgatcataagaaaggagcaaagaaagctagaattgaagaagataagaaagaaacagaggacaaaaaaacagacgttaaggaagaagataaatccaaggacaacagtggtgaaaaaacagataccaaaggaaccaaatcagaacagctcagcaacccctgaatttgacagtctcaccaatttcagaaaatcattaaaaagaaaatattgaaaggaaaatgttttctttttgaagacttctggcttcattttatactactttggcatggactgtatttattttcaaatgggactttttcgtttttgtttttctgggcaagttttattgtgagattttctaattatgaagcaaaatttcttttctccaccatgctttatgtgatagtatttaaaattgatgtgagttattatgtcaaaaaaactgatctattaaagaagtaattggcctttctgagctgaaaaaaaaaaaaaaaaag

SEQ ID NO: 96 - DNA, 1955 bp, Homo sapiens
gcggcggccgcgggcggcgggcggcggaggtgcggggcctgctcccgccggcaccatggccaagaccgtggcctatttctacgaccccgacgtgggcaacttccactacggagctggacaccctatgaagccccatcgcctggcattgacccatagcctggtcctgcattacggtctctataagaagatgatcgtcttcaagccataccaggcctcccagcatgacatgtgccgcttccactccgaggactacattgacttcctgcagagagtcagccccaccaatatgcaaggcttcaccaagagtcttaatgccttcaacgtaggcgatgactgcccagtgtttcccgggctctttgagttctgctcgcgttacacaggcgcatctctgcaaggagcaacccagctgaacaacaagatctgtgatattgccattaactgggctggtggtctgcaccatgccaagaagtttgaggcctctggcttctgctatgtcaacgacattgtgattggcatcctggagctgctcaagtaccaccctcgggtgctctacattgacattgacatccaccatggtgacggggttcaagaagctttctacctcactgaccgggtcatgacggtgtccttccacaaatacggaaattacttcttccctggcacaggtgacatgtatgaagtcggggcagagagtggccgctactactgtctgaacgtgcccctgcgggatggcattgatgaccagagttacaagcaccttttccagccggttatcaaccaggtagtggacttctaccaacccacgtgcattgtgctccagtgtggagctgactctctgggctgtgatcgattgggctgctttaacctcagcatccgagggcatggggaatgcgttgaatatgtcaagagcttcaatatccctctactcgtgctgggtggtggtggttatactgtccgaaatgttgcccgctgctggacatatgagacatcgctgctggtagaagaggccattagtgaggagcttccctatagtgaatacttcgagtactttgccccagacttcacacttcatccagatgtcagcacccgcatcgagaatcagaactcacgccagtatctggaccagatccgccagacaatctttgaaaacctgaagatgctgaaccatgcacctagtgtccagattcatgacgtgcctgcagacctcctgacctatgacaggactgatgaggctgatgcagaggagaggggtcctgaggagaactatagcaggccagaggcacccaatgagttctatgatggagaccatgacaatgacaaggaaagcgatgtggagatttaagagtggcttgggatgctgtgtcccaaggaatttcttttcacctcttggttgggctggagggaaaaggagtggctcctagagtcctgggggtcaccccagggcttttgctgactctgggaaagagtctggagaccacatttggttctcgaaccatctacctgcttttcctctctctcccaaggcctgacaatggtacctattagggatggagatacagacaaggatagctatctgggacattattggcagtgggccctggaggccagtccctagccccccttgccccttatttcttccctgcttccctcgaacccagagatttttgagggatgaacgggtagacaaggactgagattgcctctgacttcctcctcccctgggttctgacttcttcctccccttgcttccagggaagatgaagagagagagatttggaaggggctctggctccctaacacctgaatcccagatgatgggaagtatgttttcaagtgtggggaggatatgaaaatgttctgttctcacttttggctttatgtccattttaccactgtttttatccaataaactaagtcggtattttttgtacctttaaaaaaaaaaaaaaaaaaaa

SEQ ID NO: 97 - DNA, 8459 bp, Homo sapiens
ggaggttgtggggccgccgccgcggagcaccgtccccgccgccgcccgagcccgagcccgagcccgcgcacccgcccgcgccgccgccgccgccgcccgaacagcctcccagcctgggcccccggcggcgccgtggccgcgtcccggctgtcgccgcccgagcccgagcccgcgcgccggcgggtggcggcgcaggctgaggagatgcggcgcggagcgccggagcagggctagagccggccgccgccgcccgccgcggtaagcgcagccccggcccggcgcccgcgggccattgtccgccgcccgccccgcgccccgcgcagcctgcaggccttggagcccgcggcaggtggacgccgccggtccacacccgccccgcgcgcggccgtgggaggcgggggccagcgctggccgcgcgccgtgggacccgccggtccccagggccgcccggccccttctggacctttccacccgcgccgcgaggcggcttcgcccgccggggcgggggcgcgggggtgggcacggcaggcagcggcgccgtctcccggtgcggggcccgcgccccccgagcaggttcatctgcagaagccagcggacgcctctgttcaacttgtgggttacctggctcatgagaccttgccggcgaggctcggcgcttgaacgtctgtgacccagccctcaccgtcccggtacttgtatgtgttggtgggagtttggagctcgttggagctatcgtttccgtggaaattttgagccatttcgaatcacttaaaggagtggacattgctagcaatgagctcccaaagccatccagatggactttctggccgagaccagccagtggagctgctgaatcctgcccgcgtgaaccacatgcccagcacggtggatgtggccacggcgctgcctctgcaagtggccccctcggcagtgcccatggacctgcgcctggaccaccagttctcactgcctgtggcagagccggccctgcgggagcagcagctgcagcaggagctcctggcgctcaagcagaagcagcagatccagaggcagatcctcatcgctgagttccagaggcagcacgagcagctctcccggcagcacgaggcgcagctccacgagcacatcaagcaacaacaggagatgctggccatgaagcaccagcaggagctgctggaacaccagcggaagctggagaggcaccgccaggagcaggagctggagaagcagcaccgggagcagaagctgcagcagctcaagaacaaggagaagggcaaagagagtgccgtggccagcacagaagtgaagatgaagttacaagaatttgtcctcaataaaaagaaggcgctggcccaccggaatctgaaccactgcatttccagcgaccctcgctactggtacgggaaaacgcagcacagttcccttgaccagagttctccaccccagagcggagtgtcgacctcctataaccacccggtcctgggaatgtacgacgccaaagatgacttccctcttaggaaaacagcttctgaaccgaatctgaaattacggtccaggctaaagcagaaagtggccgaaagacggagcagccccctgttacgcaggaaagacgggccagtggtcactgctctaaaaaagcgtccgttggatgtcacagactccgcgtgcagcagcgccccaggctccggacccagctcacccaacaacagctccgggagcgtcagcgcggagaacggtatcgcgcccgccgtccccagcatcccggcggagacgagtttggcgcacagacttgtggcacgagaaggctcggccgctccacttcccctctacacatcgccatccttgcccaacatcacgctgggcctgcctgccaccggcccctctgcgggcacggcgggccagcaggacaccgagagactcacccttcccgccctccagcagaggctctcccttttccccggcacccacctcactccctacctgagcacctcgcccttggagcgggacggaggggcagcgcacagccctcttctgcagcacatggtcttactggagcagccaccggcacaagcacccctcgtcacaggcctgggagcactgcccctccacgcacagtccttggttggtgcagaccgggtgtccccctccatccacaagctgcggcagcaccgcccactggggcggacccagtcggccccgctgccccagaacgcccaggctctgcagcacctggtcatccagcagcagcatcagcagtttctggagaaacacaagcagcagttccagcagcagcaactgcagatgaacaagatcatccccaagccaagcgagccagcccggcagccggagagccacccggaggagacggaggaggagctccgtgagcaccaggctctgctggacgagccctacctggaccggctgccggggcagaaggaggcgcacgcacaggccggcgtgcaggtgaagcaggagcccattgagagcgatgaggaagaggcagagcccccacgggaggtggagccgggccagcgccagcccagtgagcaggagctgctcttcagacagcaagccctcctgctggagcagcagcggatccaccagctgaggaactaccaggcgtccatggaggccgccggcatccccgtgtccttcggcggccacaggcctctgtcccgggcgcagtcctcacccgcgtctgccaccttccccgtgtctgtgcaggagccccccaccaagccgaggttcacgacaggcctcgtgtatgacacgctgatgctgaagcaccagtgcacctgcgggagtagcagcagccaccccgagcacgccgggaggatccagagcatctggtcccgcctgcaggagacgggcctccggggcaaatgcgagtgcatccgcggacgcaaggccaccctggaggagctacagacggtgcactcggaagcccacaccctcctgtatggcacgaaccccctcaaccggcagaaactggacagtaagaaacttctaggctcgctcgcctccgtgttcgtccggctcccttgcggtggtgttggggtggacagtgacaccatatggaacgaggtgcactcggcgggggcagcccgcctggctgtgggctgcgtggtagagctggtcttcaaggtggccacaggggagctgaagaatggctttgctgtggtccgcccccctggacaccatgcggaggagagcacgcccatgggcttttgctacttcaactccgtggccgtggcagccaagcttctgcagcagaggttgagcgtgagcaagatcctcatcgtggactgggacgtgcaccatggaaacgggacccagcaggctttctacagcgaccctagcgtcctgtacatgtccctccaccgctacgacgatgggaacttcttcccaggcagcggggctcctgatgaggtgggcacagggcccggcgtgggtttcaacgtcaacatggctttcaccggcggcctggacccccccatgggagacgctgagtacttggcggccttcagaacggtggtcatgccgatcgccagcgagtttgccccggatgtggtgctggtgtcatcaggcttcgatgccgtggagggccaccccacccctcttgggggctacaacctctccgccagatgcttcgggtacctgacgaagcagctgatgggcctggctggcggccggattgtcctggccctcgagggaggccacgacctgaccgccatttgcgacgcctcggaagcatgtgtttctgccttgctgggaaacgagcttgatcctctcccagaaaaggttttacagcaaagacccaatgcaaacgctgtccgttccatggagaaagtcatggagatccacagcaagtactggcgctgcctgcagcgcacaacctccacagcggggcgttctctgatcgaggctcagacttgcgagaacgaagaagccgagacggtcaccgccatggcctcgctgtccgtgggcgtgaagcccgccgaaaagagaccagatgaggagcccatggaagaggagccgcccctgtagcactccctcgaagctgctgttctcttgtctgtctgtctctgtcttgaagctcagccaagaaactttcccgtgtcacgcctgcgtcccaccgtggggctctcttggagcacccagggacacccagcgtgcaacagccacgggaagcctttctgccgcccaggcccacaggtctcgagacgcacatgcacgcctgggcgtggcagcctcacagggaacacgggacagacgccggcgacgcgcagacacacggacacgcggaagccaagcacactctggcgggtcccgcaagggacgccgtggaagaaaggagcctgtggcaacaggcggccgagctgccgaattcagttgacacgaggcacagaaaacaaatatcaaagatctaataatacaaaacaaacttgattaaaactggtgcttaaagtttattacccacaactccacagtctctgtgtaaaccactcgactcatcttgtagcttattttttttttaaagaggacgttttctacggctgtggcccgcctctgtgaaccatagcggtgtgcggcggggggtctgcacccgggtgggggacagagggacctttaaagaaaacaaaactggacagaaacaggaatgtgagctgggggagctggcttgagtttctcaaaagccatcggaagatgcgagtttgtgcctttttttttattgctctggtggatttttgtggctgggttttctgaagtctgaggaacaatgccttaagaaaaaacaaacagcaggaatcggtgggacagtttcctgtggccagccgagcctggcagtgctggcaccgcgagctggcctgacgcctcaagcacgggcaccagccgtcatctccggggccaggggctgcagcccggcggtccctgttttgctttattgctgtttaagaaaaatggaggtagttccaaaaaagtggcaaatcccgttggaggttttgaagtccaacaaattttaaacgaatccaaagtgttctcacacgtcacatacgattgagcatctccatctggtcgtgaagcatgtggtaggcacacttgcagtgttacgatcggaatgctttttattaaaagcaagtagcatgaagtattgcttaaattttaggtataaataaatatatatatgtataatatatattccaatgtattccaagctaagaaacttacttgattcttatgaaatcttgataaaatatttataatgcatttatagaaaaagtatatatatatatataaaatgaatgcagattgcgaaggtccctgcaaatggatggcttgtgaatttgctctcaaggtgcttatggaaagggatcctgattgattgaaattcatgttttctcaagctccagattggctagatttcagatcgccaacacattcgccactgggcaactaccctacaagtttgtactttcattttaattattttctaacagaaccgctcccgtctccaagccttcatgcacatatgtacctaatgagtttttatagcaaagaatataaatttgctgttgatttttgtatgaattttttcacaaaaagatcctgaataagcattgttttatgaattttacatttttcctcaccatttagcaattttctgaatggtaataatgtctaaatctttttcctttctgaattcttgcttgtacatttttttttacctttcaaaggtttttaattatttttgtttttatttttgtacgatgagttttctgcagcgtacagaattgttgctgtcagattctattttcagaaagtgagaggagggaccgtaggtcttttcggagtgacaccaacgattgtgtctttcctggtctgtcctaggagctgtataaagaagcccaggggctctttttaactttcaacactagtagtattacgaggggtggtgtgtttttcccctccgtggcaagggcagggagggttgcttaggatgcccggccaccctgggaggcttgccagatgccgggggcagtcagcattaatgaaactcatgtttaaacttctctgaccacatcgtcaggatagaattctaacttgagttttccaaagaccttttgagcatgtcagcaatgcatggggcacacgtggggctctttacccacttgggtttttccactgcagccacgtggccagccctggattttggagcctgtggctgcaaggaacccagggacccttgttgcctggtgaacctgcagggagggtatgattgcctgaccaggacagccagtctttactctttttctcttcaacagtaactgacagtcacgttttactggtaacttattttccagcacatgaagccaccagtttcattccaaagtgtatattgggttcagacttgggggcagaagttcagacacaccgtgctcaggagggacccagagccgagtttcggagtttggtaaagtttacagggtagcttctgaaattaactcaaacttttgaccaaatgagtgcagattcttggattcacttggtcactgggctgctgatggtcagctctgagacagtggtttgagagcaggcagaacggtcttgggacttgtttgactttcccctccctggtggccactctttgctctgaagcccagattggcaagaggagctggtccattccccattcatggcacagagcagtggcagggcccagctagcaggctcttctggcctccttggcctcattctctgcatagccctctggggatcctgccacctgccctcttaccccgccgtggcttatggggaggaatgcatcatctcactttttttttttaagcagatgatgggataacatggactgctcagtggccaggttatcagtggggggacttaattctaatctcattcaaatggagacgccctctgcaaaggcctggcagggggaggcacgtttcatctgtcagctcactccagcttcacaaatgtgctgagagcattactgtgtagccttttctttgaagacacactcggctcttctccacagcaagcgtccagggcagatggcagaggatctgcctcggcgtctgcaggcgggaccacgtcagggagggttccttcatgtgttctccctgtgggtccttggacctttagcctttttcttcctttgcaaaggccttgggggcactggctgggagtcagcaagcgagcactttatatccctttgagggaaaccctgatgacgccactgggcctcttggcgtctgccctgccctcgcggcttcccgccgtgccgcagcgtgcccacgtgcccacgccccaccagcaggcggctgtcccggaggccgtggcccgctgggactggccgcccctccccagcgtcccagggctctggttctggagggccactttgtcaaggtgtttcagtttttctttacttcttttgaaaatctgtttgcaaggggaaggaccatttcgtaatggtctgacacaaaagcaagtttgatttttgcagcactagcaatggactttgttgtttttctttttgatcagaacattccttctttactggtcacagccacgtgctcattccattcttctttttgtagactttgggcccacgtgttttatgggcattgatacatatataaatatatagatataaatatatatgaatatatttttttaagtttcctacacctggaggttgcatggactgtacgaccggcatgactttatattgtatacagattttgcacgccaaactcggcagctttggggaagaagaaaaatgcctttctgttcccctctcatgacatttgcagatacaaaagatggaaatttttctgtaaaacaaaaccttgaaggagaggagggcggggaagtttgcgtcttattgaacttattcttaagaaattgtactttttattgtaagaaaaataaaaaggactacttaaacatttgtcatattaagaaaaaaagtttatctagcacttgtgacataccaataatagagtttattgtatttatgtggaaacagtgttttagggaaactactcagaattcacagtgaactgcctgtctctctcgagttgatttggaggaattttgttttgttttgttttgtttgtttccttttatctccttccacgggccaggcgagcgccgcccgccctcactggccttgtgacggtttattctgattgagaactgggcggactcgaaagagtccccttttccgcacagctgtgttgactttttaattacttttaggtgatgtatggctaagatttcactttaagcagtcgtgaactgtgcgagcactgtggtttacaattatactttgcatcgaaaggaaaccatttcttcattgtaacgaagctgagcgtgttcttagctcggcctcactttgtctctggcattgattaaaagtctgctattgaaagaaaaag

SEQ ID NO: 98 - DNA, 3449 bp, Homo sapiens
atgaactctcccaacgagtcggatgggatgtcaggtcgggaaccatccttggaaatcctgccgcggacttctctgcacagcatccctgtgacagtggaggtgaagccggtgctgccaagagccatgcccagttccatggggggtgggggtggaggcagccccagccctgtggagctacggggggctctggtgggctctgtggaccccacactgcgggagcagcaactgcagcaggagctcctggcgctcaagcagcagcagcagctgcagaagcagctcctgttcgctgagttccagaaacagcatgaccacctgacaaggcagcatgaggtccagctgcagaagcacctcaagcagcagcaggagatgctggcagccaagcagcagcaggagatgctggcagccaagcggcagcaggagctggagcagcagcggcagcgggagcagcagcggcaggaagagctggagaagcagcggctggagcagcagctgctcatcctgcggaacaaggagaagagcaaagagagtgccattgccagcactgaggtaaagctgaggctccaggaattcctcttgtcgaagtcaaaggagcccacaccaggcggcctcaaccattccctcccacagcaccccaaatgctggggagcccaccatgcttctttggaccagagttcccctccccagagcggcccccctgggacgcctccctcctacaaactgcctttgcctgggccctacgacagtcgagacgacttccccctccgcaaaacagcctctgaacccaacttgaaagtgcgttcaaggctaaaacagaaggtggctgagcggagaagcagtcccctcctgcgtcgcaaggatgggactgttattagcacctttaagaagagagctgttgagatcacaggtgccgggcctggggcgtcgtccgtgtgtaacagcgcacccggctccggccccagctctcccaacagctcccacagcaccatcgctgagaatggctttactggctcagtccccaacatccccactgagatgctccctcagcaccgagccctccctctggacagctcccccaaccagttcagcctctacacgtctccttctctgcccaacatctccctagggctgcaggccacggtcactgtcaccaactcacacctcactgcctccccgaagctgtcgacacagcaggaggccgagaggcaggccctccagtccctgcggcagggtggcacgctgaccggcaagttcatgagcacatcctctattcctggctgcctgctgggcgtggcactggagggcgacgggagcccccacgggcatgcctccctgctgcagcatgtgctgttgctggagcaggcccggcagcagagcaccctcattgctgtgccactccacgggcagtccccactagtgacgggtgaacgtgtggccaccagcatgcggacggtaggcaagctcccgcggcatcggcccctgagccgcactcagtcctcaccgctgccgcagagtccccaggccctgcagcagctggtcatgcaacaacagcaccagcagttcctggagaagcagaagcagcagcagctacagctgggcaagatcctcaccaagacaggggagctgcccaggcagcccaccacccaccctgaggagacagaggaggagctgacggagcagcaggaggtcttgctgggggagggagccctgaccatgccccgggagggctccacagagagtgagagcacacaggaagacctggaggaggaggacgaggaagaggatggggaggaggaggaggattgcatccaggttaaggacgaggagggcgagagtggtgctgaggaggggcccgacttggaggagcctggtgctggatacaaaaaactgttctcagatgcccagccgctgcagcctttgcaggtgtaccaggcgcccctcagcctggccactgtgccccaccaggccctgggccgtacccagtcctcccctgctgcccctgggggcatgaagagccccccagaccagcccgtcaagcacctcttcaccacaggtgtggtctacgacacgttcatgctaaagcaccagtgcatgtgcgggaacacacacgtgcaccctgagcatgctggccggatccagagcatctggtcccggctgcaggagacaggcctgcttagcaagtgcgagcggatccgaggtcgcaaagccacgctagatgagatccagacagtgcactctgaataccacaccctgctctatgggaccagtcccctcaaccggcagaagctagacagcaagaagttgctcggccccatcagccagaagatgtatgctgtgctgccttgtgggggcatcggggtggacagtgacaccgtgtggaatgagatgcactcctccagtgctgtgcgcatggcagtgggctgcctgctggagctggccttcaaggtggctgcaggagagctcaagaatggatttgccatcatccggcccccaggacaccacgccgaggaatccacagccatgggattctgcttcttcaactctgtagccatcaccgcaaaactcctacagcagaagttgaacgtgggcaaggtcctcatcgtggactgggacattcaccatggcaatggcacccagcaggcgttctataatgacccctctgtgctctacatctctctgcatcgctatgacaacgggaacttctttccaggctctggggctcctgaagaggttggtggaggaccaggcgtggggtacaatgtgaacgtggcatggacaggaggtgtggacccccccattggagacgtggagtaccttacagccttcaggacagtggtgatgcccattgcccacgagttctcacctgatgtggtcctagtctccgccgggtttgatgctgttgaaggacatctgtctcctctgggtggctactctgtcaccgccagatgttttggccacttgaccaggcagctgatgaccctggcagggggccgggtggtgctggccctggagggaggccatgacttgaccgccatctgtgatgcctctgaggcttgtgtctcggctctgctcagtgtagagctgcagcccttggatgaggcagtcttgcagcaaaagcccaacatcaacgcagtggccacgctagagaaagtcatcgagatccagagcaaacactggagctgtgtgcagaagttcgccgctggtctgggccggtccctgcgagaggcccaagcaggtgagaccgaggaggccgagactgtgagcgccatggccttgctgtcggtgggggccgagcaggcccaggctgcggcagcccgggaacacagccccaggccggcagaggagcccatggagcaggagcctgccctgtgacgccccggcccccatccctctgggcttcaccattgtgattttgtttattttttctattaaaaacaaaaagtcacacattc

SEQ ID NO: 99 - DNA, 4099 bp, Homo sapiens
gggcagtcccctgaggagcggggctggttgaaacgctaggggcgggatctggcggagtggaagaaccgcggcaggggccaagcctcctcaactatgacctcaaccggccaggattccaccacaaccaggcagcgaagaagtaggcagaacccccagtcgccccctcaggactccagtgtcacttcgaagcgaaatattaaaaagggagccgttccccgctctatccccaatctagcggaggtaaagaagaaaggcaaaatgaagaagctcggccaagcaatggaagaagacctaatcgtgggactgcaagggatggatctgaaccttgaggctgaagcactggctggcactggcttggtgttggatgagcagttaaatgaattccattgcctctgggatgacagcttcccggaaggccctgagcggctccatgccatcaaggagcaactgatccaggagggcctcctagatcgctgcgtgtcctttcaggcccggtttgctgaaaaggaagagctgatgttggttcacagcctagaatatattgatctgatggaaacaacccagtacatgaatgagggagaactccgtgtcctagcagacacctacgactcagtttatctgcatccgaactcatactcctgtgcctgcctggcctcaggctctgtcctcaggctggtggatgcggtcctgggggctgagatccggaatggcatggccatcattaggcctcctggacatcacgcccagcacagtcttatggatggctattgcatgttcaaccacgtggctgtggcagcccgctatgctcaacagaaacaccgcatccggagggtccttatcgtagattgggatgtgcaccacggtcaaggaacacagttcaccttcgaccaggaccccagtgtcctctatttctccatccaccgctacgagcagggtaggttctggccccacctgaaggcctctaactggtccaccacaggtttcggccaaggccaaggatataccatcaatgtgccttggaaccaggtggggatgcgggatgctgactacattgctgctttcctgcacgtcctgctgccagtcgccctcgagttccagcctcagctggtcctggtggctgctggatttgatgccctgcaaggggaccccaagggtgagatggccgccactccggcagggttcgcccagctaacccacctgctcatgggtctggcaggaggcaagctgatcctgtctctggagggtggctacaacctccgcgccctggctgaaggcgtcagtgcttcgctccacacccttctgggagacccttgccccatgctggagtcacctggtgccccctgccggagtgcccaggcttcagtttcctgtgctctggaagcccttgagcccttctgggaggttcttgtgagatcaactgagaccgtggagagggacaacatggaggaggacaatgtagaggagagcgaggaggaaggaccctgggagccccctgtgctcccaatcctgacatggccagtgctacagtctcgcacagggctggtctatgaccaaaatatgatgaatcactgcaacttgtgggacagccaccaccctgaggtaccccagcgcatcttgcggatcatgtgccgtctggaggagctgggccttgccgggcgctgcctcaccctgacaccgcgccctgccacagaggctgagctgctcacctgtcacagtgctgagtacgtgggtcatctccgggccacagagaaaatgaaaacccgggagctgcaccgtgagagttccaactttgactccatctatatctgccccagtaccttcgcctgtgcacagcttgccactggcgctgcctgccgcctggtggaggctgtgctctcaggagaggttctgaatggtgctgctgtggtgcgtcccccaggacaccacgcagagcaggatgcagcttgcggtttttgctttttcaactctgtggctgtggctgctcgccatgcccagactatcagtgggcatgccctacggatcctgattgtggattgggatgtccaccacggtaatggaactcagcacatgtttgaggatgaccccagtgtgctatatgtgtccctgcaccgctatgatcatggcaccttcttccccatgggggatgagggtgccagcagccagatcggccgggctgcgggcacaggcttcaccgtcaacgtggcatggaacgggccccgcatgggtgatgctgactacctagctgcctggcatcgcctggtgcttcccattgcctacgagtttaacccagaactggtgctggtctcagctggctttgatgctgcacggggggatccgctggggggctgccaggtgtcacctgagggttatgcccacctcacccacctgctgatgggccttgccagtggccgcattatccttatcctagagggtggctataacctgacatccatctcagagtccatggctgcctgcactcgctccctccttggagacccaccacccctgctgaccctgccacggcccccactatcaggggccctggcctcaatcactgagaccatccaagtccatcgcagatactggcgcagcttacgggtcatgaaggtagaagacagagaaggaccctccagttctaagttggtcaccaagaaggcaccccaaccagccaaacctaggttagctgagcggatgaccacacgagaaaagaaggttctggaagcaggcatggggaaagtcacctcggcatcatttggggaagagtccactccaggccagactaactcagagacagctgtggtggccctcactcaggaccagccctcagaggcagccacagggggagccactctggcccagaccatttctgaggcagccattgggggagccatgctgggccagaccacctcagaggaggctgtcgggggagccactccggaccagaccacctcagaggagactgtgggaggagccattctggaccagaccacctcagaggatgctgttgggggagccacgctgggccagactacctcagaggaggctgtaggaggagctacactggcccagaccacctcggaggcagccatggagggagccacactggaccagactacgtcagaggaggctccagggggcaccgagctgatccaaactcctctagcctcgagcacagaccaccagacccccccaacctcacctgtgcagggaactacaccccagatatctcccagtacactgattgggagtctcaggaccttggagctaggcagcgaatctcagggggcctcagaatctcaggccccaggagaggagaacctactaggagaggcagctggaggtcaggacatggctgattcgatgctgatgcagggatctaggggcctcactgatcaggccatattttatgctgtgacaccactgccctggtgtccccatttggtggcagtatgccccatacctgcagcaggcctagacgtgacccaaccttgtggggactgtggaacaatccaagagaattgggtgtgtctctcttgctatcaggtctactgtggtcgttacatcaatggccacatgctccaacaccatggaaattctggacacccgctggtcctcagctacatcgacctgtcagcctggtgttactactgtcaggcctatgtccaccaccaggctctcctagatgtgaagaacatcgcccaccagaacaagtttggggaggatatgccccacccacactaagccccagaatacggtccctcttcaccttctgaggcccacgatagaccagctgtagctcattccagcctgtaccttggatgaggggtagcctcccactgcatcccatcctgaatatcctttgcaactccccaagagtgcttatttaagtgttaatacttttaagagaactgcgacgattaattgtggatctccccctgcccattgcctgcttgaggggcaccactactccagcccagaaggaaaggggggcagctcagtggccccaagagggagctgatatcatgaggataacattggcgggaggggagttaactggcaggcatggcaaggttgcatatgtaataaagtacaagctgtt

SEQ ID NO: 100 - DNA, 4206 bp, Homo sapiens
ataatacctaccttgcaggaccacgacaggattaagtgaggaaaaacccccatgagagtgttttgccattgtcaagtgagcctgagggaggctgaggggggatcaggctgtatcatgcccccgaggacaaactttccagtttaccctgctccctctctctgtccctaggctgccccaggccctgtgcagacacaccaggccctcagccgcagcccatggacctgcgggtgggccagcggcccccagtggagcccccaccagagcccacattgctggccctgcagcgtccccagcgcctgcaccaccacctcttcctagcaggcctgcagcagcagcgctcggtggagcccatgaggctctccatggacacgccgatgcccgagttgcaggtgggaccccaggaacaagagctgcggcagcttctccacaaggacaagagcaagcgaagtgctgtagccagcagcgtggtcaagcagaagctagcggaggtgattctgaaaaaacagcaggcggccctagaaagaacagtccatcccaacagccccggcattccctacagaaccctggagcccctggagacggaaggagccacccgctccatgctcagcagctttttgcctcctgttcccagcctgcccagtgaccccccagagcacttccctctgcgcaagacagtctctgagcccaacctgaagctgcgctataagcccaagaagtccctggagcggaggaagaatccactgctccgaaaggagagtgcgccccccagcctccggcggcggcccgcagagaccctcggagactcctccccaagtagtagcagcacgcccgcatcagggtgcagctcccccaatgacagcgagcacggccccaatcccatcctgggctcggaggcgctcttgggccagcggctgcggctgcaggagacttctgtggccccgttcgccttgccgacagtgtccttgctgcccgcaatcactctggggctgcccgcccctgccagggctgacagtgaccgcaggacccatccgactctgggccctcgggggccaatcctggggagcccccacactcccctcttcctgccccatggcttggagcccgaggctgggggcaccttgccctctcgcctgcagcccattctcctcctggacccctcaggctctcatgccccgctgctgactgtgcccgggcttgggcccttgcccttccactttgcccagtccttaatgaccaccgagcggctctctgggtcaggcctccactggccactgagccggactcgctcagagcccctgccccccagtgccaccgctcccccaccgccgggccccatgcagccccgcctggagcagctcaaaactcacgtccaggtgatcaagaggtcagccaagccgagtgagaagccccggctgcggcagataccctcggctgaagacctggagacagatggcgggggaccgggccaggtggtggacgatggcctggagcacagggagctgggccatgggcagcctgaggccagaggccccgctcctctccagcagcaccctcaggtgttgctctgggaacagcagcgactggctgggcggctcccccggggcagcaccggggacactgtgctgcttcctctggcccagggtgggcaccggcctctgtcccgggctcagtcttccccagccgcacctgcctcactgtcagccccagagcctgccagccaggcccgagtcctctccagctcagagacccctgccaggaccctgcccttcaccacagggctgatctatgactcggtcatgctgaagcaccagtgctcctgcggtgacaacagcaggcacccggagcacgccggccgcatccagagcatctggtcccggctgcaggagcgggggctccggagccagtgtgagtgtctccgaggccggaaggcctccctggaagagctgcagtcggtccactctgagcggcacgtgctcctctacggcaccaacccgctcagccgcctcaaactggacaacgggaagctggcagggctcctggcacagcggatgtttgtgatgctgccctgtggtggggttggggtggacactgacaccatctggaatgagcttcattcctccaatgcagcccgctgggccgctggcagtgtcactgacctcgccttcaaagtggcttctcgtgagctaaagaatggtttcgctgtggtgcggcccccaggacaccatgcagatcattcaacagccatgggcttctgcttcttcaactcagtggccatcgcctgccggcagctgcaacagcagagcaaggccagcaagatcctcattgtagactgggacgtgcaccatggcaacggcacccagcaaaccttctaccaagaccccagtgtgctctacatctccctgcatcgccatgacgacggcaacttcttcccggggagtggggctgtggatgaggtaggggctggcagcggtgagggcttcaatgtcaatgtggcctgggctggaggtctggacccccccatgggggatcctgagtacctggctgctttcaggatagtcgtgatgcccatcgcccgagagttctctccagacctagtcctggtgtctgctggatttgatgctgctgagggtcacccggccccactgggtggctaccatgtttctgccaaatgttttggatacatgacgcagcaactgatgaacctggcaggaggcgcagtggtgctggccttggagggtggccatgacctcacagccatctgtgacgcctctgaggcctgtgtggctgctcttctgggtaacagggtggatcccctttcagaagaaggctggaaacagaaacccaacctcaatgccatccgctctctggaggccgtgatccgggtgcacagtaaatactggggctgcatgcagcgcctggcctcctgtccagactcctgggtgcctagagtgccaggggctgacaaagaagaagtggaggcagtgaccgcactggcgtccctctctgtgggcatcctggctgaagataggccctcggagcagctggtggaggaggaagaacctatgaatctctaaggctctggaaccatctgcccgcccaccatgcccttgggacctggttctcttctaacccctggcaatagcccccattcctgggtctttagagatcctgtgggcaagtagttggaaccagagaacagcctgcctgctttgacagttatcccagggagcgtgagaaaatccctgggtctagaatgggaactggagaggaccctgagaggagacgggctgggcggcgacccccacagggctctcgagaacagattctcccctccagtatgggccctggctgtggcccccattcctcaggactgcacagaggaggactggctccggctccgtcgggctcacccttaaccactattcctggctctgcaaaccccagactttgcacacagccccaggctccacacagaaatgtgaacttggcctcagacaggctggcccttcctaggctctaggggctaggggggagtggggagccaagaggtcccatattcctgagtgcaggggtagtccctctcacctgcttcctcagacgactctggaagcttccctctaccaccgggcactgagacgaagctccctgacagccgagactggcagccctccatctggtccgtaccctcgccagaggcccccctacatcaacctcctggcgatgccctggtggagcagatgggtgctctgggagtcctgtgcttcctgatccaatggtgccaaacccttcatctcccccagaagcgcagcatacccctgggacccctcggccactgcccactcggggagccttctctgtttctggggcctcccccaccatagctctgattcccaccccacataggaatagcctgactgagggggaaggggtgggagagaagatacagacatggaggaggggaggctgctctggcaaagtcttcaaggcttttgggggtccaggcctggggtcaagaaggaaaatgtgtgtgagcatgtgtgtgagtgaggcgtgtgtgtgagcgtgtgtgtgagtgaggcgtgtgtgtgtgtctttcctaggacccaccataccctgtgtatgtatgcatgtttttgtaaaaaggaagaaaatggaaaaaaatctgaacaataaatgttttatttgctttaaaaaaaaaaaaaaaaa

SEQ ID NO: 101 - DNA, 4086 bp, Homo sapiens
ataatacctaccttgcaggaccacgacaggattaagtgaggaaaaacccccatgagagtgttttgccattgtcaagtgagcctgagggaggctgaggggggatcaggctgtatcatgcccccgaggacaaactttccagtttaccctgctccctctctctgtccctaggctgccccaggccctgtgcagacacaccaggccctcagccgcagcccatggacctgcgggtgggccagcggcccccagtggagcccccaccagagcccacattgctggccctgcagcgtccccagcgcctgcaccaccacctcttcctagcaggcctgcagcagcagcgctcggtggagcccatgaggctctccatggacacgccgatgcccgagttgcaggtgggaccccaggaacaagagctgcggcagcttctccacaaggacaagagcaagcgaagtgctgtagccagcagcgtggtcaagcagaagctagcggaggtgattctgaaaaaacagcaggcggccctagaaagaacagtccatcccaacagccccggcattccctacagaaccctggagcccctggagacggaaggagccacccgctccatgctcagcagctttttgcctcctgttcccagcctgcccagtgaccccccagagcacttccctctgcgcaagacagtctctgagcccaacctgaagctgcgctataagcccaagaagtccctggagcggaggaagaatccactgctccgaaaggagagtgcgccccccagcctccggcggcggcccgcagagaccctcggagactcctccccaagtagtagcagcacgcccgcatcagggtgcagctcccccaatgacagcgagcacggccccaatcccatcctgggcgacagtgaccgcaggacccatccgactctgggccctcgggggccaatcctggggagcccccacactcccctcttcctgccccatggcttggagcccgaggctgggggcaccttgccctctcgcctgcagcccattctcctcctggacccctcaggctctcatgccccgctgctgactgtgcccgggcttgggcccttgcccttccactttgcccagtccttaatgaccaccgagcggctctctgggtcaggcctccactggccactgagccggactcgctcagagcccctgccccccagtgccaccgctcccccaccgccgggccccatgcagccccgcctggagcagctcaaaactcacgtccaggtgatcaagaggtcagccaagccgagtgagaagccccggctgcggcagataccctcggctgaagacctggagacagatggcgggggaccgggccaggtggtggacgatggcctggagcacagggagctgggccatgggcagcctgaggccagaggccccgctcctctccagcagcaccctcaggtgttgctctgggaacagcagcgactggctgggcggctcccccggggcagcaccggggacactgtgctgcttcctctggcccagggtgggcaccggcctctgtcccgggctcagtcttccccagccgcacctgcctcactgtcagccccagagcctgccagccaggcccgagtcctctccagctcagagacccctgccaggaccctgcccttcaccacagggctgatctatgactcggtcatgctgaagcaccagtgctcctgcggtgacaacagcaggcacccggagcacgccggccgcatccagagcatctggtcccggctgcaggagcgggggctccggagccagtgtgagtgtctccgaggccggaaggcctccctggaagagctgcagtcggtccactctgagcggcacgtgctcctctacggcaccaacccgctcagccgcctcaaactggacaacgggaagctggcagggctcctggcacagcggatgtttgtgatgctgccctgtggtggggttggggtggacactgacaccatctggaatgagcttcattcctccaatgcagcccgctgggccgctggcagtgtcactgacctcgccttcaaagtggcttctcgtgagctaaagaatggtttcgctgtggtgcggcccccaggacaccatgcagatcattcaacagccatgggcttctgcttcttcaactcagtggccatcgcctgccggcagctgcaacagcagagcaaggccagcaagatcctcattgtagactgggacgtgcaccatggcaacggcacccagcaaaccttctaccaagaccccagtgtgctctacatctccctgcatcgccatgacgacggcaacttcttcccggggagtggggctgtggatgaggtaggggctggcagcggtgagggcttcaatgtcaatgtggcctgggctggaggtctggacccccccatgggggatcctgagtacctggctgctttcaggatagtcgtgatgcccatcgcccgagagttctctccagacctagtcctggtgtctgctggatttgatgctgctgagggtcacccggccccactgggtggctaccatgtttctgccaaatgttttggatacatgacgcagcaactgatgaacctggcaggaggcgcagtggtgctggccttggagggtggccatgacctcacagccatctgtgacgcctctgaggcctgtgtggctgctcttctgggtaacagggtggatcccctttcagaagaaggctggaaacagaaacccaacctcaatgccatccgctctctggaggccgtgatccgggtgcacagtaaatactggggctgcatgcagcgcctggcctcctgtccagactcctgggtgcctagagtgccaggggctgacaaagaagaagtggaggcagtgaccgcactggcgtccctctctgtgggcatcctggctgaagataggccctcggagcagctggtggaggaggaagaacctatgaatctctaaggctctggaaccatctgcccgcccaccatgcccttgggacctggttctcttctaacccctggcaatagcccccattcctgggtctttagagatcctgtgggcaagtagttggaaccagagaacagcctgcctgctttgacagttatcccagggagcgtgagaaaatccctgggtctagaatgggaactggagaggaccctgagaggagacgggctgggcggcgacccccacagggctctcgagaacagattctcccctccagtatgggccctggctgtggcccccattcctcaggactgcacagaggaggactggctccggctccgtcgggctcacccttaaccactattcctggctctgcaaaccccagactttgcacacagccccaggctccacacagaaatgtgaacttggcctcagacaggctggcccttcctaggctctaggggctaggggggagtggggagccaagaggtcccatattcctgagtgcaggggtagtccctctcacctgcttcctcagacgactctggaagcttccctctaccaccgggcactgagacgaagctccctgacagccgagactggcagccctccatctggtccgtaccctcgccagaggcccccctacatcaacctcctggcgatgccctggtggagcagatgggtgctctgggagtcctgtgcttcctgatccaatggtgccaaacccttcatctcccccagaagcgcagcatacccctgggacccctcggccactgcccactcggggagccttctctgtttctggggcctcccccaccatagctctgattcccaccccacataggaatagcctgactgagggggaaggggtgggagagaagatacagacatggaggaggggaggctgctctggcaaagtcttcaaggcttttgggggtccaggcctggggtcaagaaggaaaatgtgtgtgagcatgtgtgtgagtgaggcgtgtgtgtgagcgtgtgtgtgagtgaggcgtgtgtgtgtgtctttcctaggacccaccataccctgtgtatgtatgcatgtttttgtaaaaaggaagaaaatggaaaaaaatctgaacaataaatgttttatttgctttaaaaaaaaaaaaaaaaa

SEQ ID NO: 102 - DNA, 4238 bp, Homo sapiens
ggggaagagaggcacagacacagataggagaagggcaccggctggagccacttgcaggactgagggtttttgcaacaaaaccctagcagcctgaagaactctaagccagatggggtggctggacgagagcagctcttggctcagcaaagaatgcacagtatgatcagctcagtggatgtgaagtcagaagttcctgtgggcctggagcccatctcacctttagacctaaggacagacctcaggatgatgatgcccgtggtggaccctgttgtccgtgagaagcaattgcagcaggaattacttcttatccagcagcagcaacaaatccagaagcagcttctgatagcagagtttcagaaacagcatgagaacttgacacggcagcaccaggctcagcttcaggagcatatcaaggaacttctagccataaaacagcaacaagaactcctagaaaaggagcagaaactggagcagcagaggcaagaacaggaagtagagaggcatcgcagagaacagcagcttcctcctctcagaggcaaagatagaggacgagaaagggcagtggcaagtacagaagtaaagcagaagcttcaagagttcctactgagtaaatcagcaacgaaagacactccaactaatggaaaaaatcattccgtgagccgccatcccaagctctggtacacggctgcccaccacacatcattggatcaaagctctccaccccttagtggaacatctccatcctacaagtacacattaccaggagcacaagatgcaaaggatgatttcccccttcgaaaaactgcctctgagcccaacttgaaggtgcggtccaggttaaaacagaaagtggcagagaggagaagcagccccttactcaggcggaaggatggaaatgttgtcacttcattcaagaagcgaatgtttgaggtgacagaatcctcagtcagtagcagttctccaggctctggtcccagttcaccaaacaatgggccaactggaagtgttactgaaaatgagacttcggttttgccccctacccctcatgccgagcaaatggtttcacagcaacgcattctaattcatgaagattccatgaacctgctaagtctttatacctctccttctttgcccaacattaccttggggcttcccgcagtgccatcccagctcaatgcttcgaattcactcaaagaaaagcagaagtgtgagacgcagacgcttaggcaaggtgttcctctgcctgggcagtatggaggcagcatcccggcatcttccagccaccctcatgttactttagagggaaagccacccaacagcagccaccaggctctcctgcagcatttattattgaaagaacaaatgcgacagcaaaagcttcttgtagctggtggagttcccttacatcctcagtctcccttggcaacaaaagagagaatttcacctggcattagaggtacccacaaattgccccgtcacagacccctgaaccgaacccagtctgcacctttgcctcagagcacgttggctcagctggtcattcaacagcaacaccagcaattcttggagaagcagaagcaataccagcagcagatccacatgaacaaactgctttcgaaatctattgaacaactgaagcaaccaggcagtcaccttgaggaagcagaggaagagcttcagggggaccaggcgatgcaggaagacagagcgccctctagtggcaacagcactaggagcgacagcagtgcttgtgtggatgacacactgggacaagttggggctgtgaaggtcaaggaggaaccagtggacagtgatgaagatgctcagatccaggaaatggaatctggggagcaggctgcttttatgcaacaggtaataggcaaagatttagctccaggatttgtaattaaagtcattatctgaacatgaaatgcattgcaggtttggtaaatggatatgatttcctatcagtttatatttctctatgatttgagttcagtgtttaaggattctacctaatgcagatatatgtatatatctatatagaggtctttctatatactgatctctatatagatatcaatgtttcattgaaaatccactggtaaggaaatacctgttatactaaaattatgatacataatatctgagcagttaataggctttaaatttatcccaaagcctgctacaccaattacttctaaagaaaacaaattcactgttattttgagtttatgtgttgagatcagtgactgctggatagtctcccagtctgatcaatgaagcattcgattagtttttgattttttgcaacatctagaatttaattttcacatcactgtacataatgtatcatactatagtcttgaacactgttaaaggtagtctgccccttccttcctctctctttttttagttaagtagaaatgttctggtcaccatgccagtagtcctaggttattgtgtaggttgcaattgaacatattaggaatacaggtggttttaaatatatagatgcaaattgcagcactactttaaatattagattatgtctcacatagcactgctcattttacttttattttgtgtaatttgatgacactgtctatcaaaaaagagcaaatgaagcagatgcaaatgttagtgagaagtaatgtgcagcattatggtccaatcagatacaatattgtgtctacaattgcaaaaaacacagtaacaggatgaatattatctgatatcaagtcaaaatcagtttgaaaagaaggtgtatcatattttatattgtcactagaatctcttaagtataattccataatgacatgggcatataccgtaacattctggcaaataacaattagaaaagataggtttaacaaaaaaatttacttgtatataatgcaccttcaggaggactatgtcctttgatgctataaaatacaaacaactttgaaggcaacagaagacactgtttattcaagtcagttctttgtcaggttcctgctgttctcctacagaaaagtgattctgtgagggtgaacaggaaatgccttgtggaaacaggaagtccaagtgattcatgtactgaggaatgtaggaaaaaaaatctgaggatagtgctttactctttctgtttttaaagggcactctatgaattgatttattgtctaagaaaataacaccacaagtagggaaattgttacggaagcttttcactggaacatttccttcatattcccttttgatatgtttaccttgttttataggtttacttttgttaagctagttaaaggttcgttgtattaagacccctttaatatggataatccaaattgacctagaatctttgtgaggttttttctattaaaatatttatatttctaaatccgaggtatttcaaggtgtagtatcctatttcaaaggagatatagcagttttgccaaatgtagacattgttcaactgtatgttattggcacgtgttgtttacattttgctgtgacatttaaaaatatttctttaaaaatgttactgctaaagatacattatccttttttaaaaagtctccattcaaattaaattaacataactagaagttagaaagtttaaaagttttccacataatgaaagtccttctgataatttgacaaatagctataataggaacactccctatcaccaacatattttggttagtatattccttcatattaaaatgactttttgtcagttgttttgcattaaaaatatggcatgcctaagataaaattgtatattttttccatctcataaatattcattttcttcaaagtcttttttcaatctcataaaaaagggatagtgcatcttttaaaatacattttatttggggaggaacatgtggctgagcagacttttgtataatattacttcaaagatatgtaatcacaaacaaaaaaaactattttttataatgtcatttgagagagtttcatcagtacagttggtggacgttaattgtttgaatttgatagtctttgaatttaatcaagaaactacctggaaccagtgaaaaggaaagctggacttaaataatcttagaattaattgataaatgtctcttttaaaatctactgtatttattataatttacacccttgaaggtgatctcttgttttgtgttgtaaatatattgtttgtatgtttcccttcttgccttctgttataagtctcttcctttctcaaataaagttttttttaaaag

SEQ ID NO: 103 - DNA, 1743 bp, Homo sapiens
gccagatctggaaggtggctgcggaacggttttaagcggaagatggaggagccggaggaaccggcggacagtgggcagtcgctggtcccggtttatatctatagtcccgagtatgtcagtatgtgtgactccctggccaagatccccaaacgggccagtatggtgcattctttgattgaagcatatgcactgcataagcagatgaggatagttaagcctaaagtggcctccatggaggagatggccaccttccacactgatgcttatctgcagcatctccagaaggtcagccaagagggcgatgatgatcatccggactccatagaatatgggctaggttatgactgcccagccactgaagggatatttgactatgcagcagctataggaggggctacgatcacagctgcccaatgcctgattgacggaatgtgcaaagtagcaattaactggtctggagggtggcatcatgcaaagaaagatgaagcatctggtttttgttatctcaatgatgctgtcctgggaatattacgattgcgacggaaatttgagcgtattctctacgtggatttggatctgcaccatggagatggtgtagaagacgcattcagtttcacctccaaagtcatgaccgtgtccctgcacaaattctccccaggatttttcccaggaacaggtgacgtgtctgatgttggcctagggaagggacggtactacagtgtaaatgtgcccattcaggatggcatacaagatgaaaaatattaccagatctgtgaaagtgtactaaaggaagtataccaagcctttaatcccaaagcagtggtcttacagctgggagctgacacaatagctggggatcccatgtgctcctttaacatgactccagtgggaattggcaagtgtcttaagtacatccttcaatggcagttggcaacactcattttgggaggaggaggctataaccttgccaacacggctcgatgctggacatacttgaccggggtcatcctagggaaaacactatcctctgagatcccagatcatgagtttttcacagcatatggtcctgattatgtgctggaaatcacgccaagctgccggccagaccgcaatgagccccaccgaatccaacaaatcctcaactacatcaaagggaatctgaagcatgtggtctagttgacagaaagagatcaggtttccagagctgaggagtggtgcctataatgaagacagcgtgtttatgcaagcagtttgtggaatttgtgactgcagggaaaatttgaaagaaattacttcctgaaaatttccaaggggcatcaagtggcagctggcttcctggggtgaagaggcaggcaccccagagtcctcaactggacctaggggaagaaggagatatcccacatttaaagttcttatttaaaaaaacacacacacacaaatgaaatttttaatctttgaaaattatttttaagcgaattggggaggggagtattttaatcatcttaaatgaaacagatcagaagctggatgagagcagtcaccagtttgtagggcaggaggcagctgagaggcagggtttgggcctcaggaccatccaggtggagccctgggagagagggtactgatcagcagactgggaggtggggagaagtccgctggtgttgttttagtgttatatatctttggtttttttaataaaatctttgaaaacctaaaaaaaaaaaaaaaaaaaaaaaaaaaaaa

SEQ ID NO: 104 - DNA, 1963 bp, Homo sapiens
gtgttgtacgaaagcgcgtctgcggccgcaatgtctgctgagagttgtagttctgtgccctatcacggccactcccatttctggtgccgtcacgggacagagcagtcggtgacaggacagagcagtcggtgacgggacacagtggttggtgacgggacagagcggtcggtgacagcctcaagggcttcagcaccgcgcccatggcagagccagacccctctcaccctctggagacccaggcagggaaggtgcaggaggctcaggactcagattcagactctgagggaggagccgctggtggagaagcagacatggacttcctgcggaacttattctcccagacgctcagcctgggcagccagaaggagcgtctgctggacgagctgaccttggaaggggtggcccggtacatgcagagcgaacgctgtcgcagagtcatctgtttggtgggagctggaatctccacatccgcaggcatccccgactttcgctctccatccaccggcctctatgacaacctagagaagtaccatcttccctacccagaggccatctttgagatcagctatttcaagaaacatccggaacccttcttcgccctcgccaaggaactctatcctgggcagttcaagccaaccatctgtcactacttcatgcgcctgctgaaggacaaggggctactcctgcgctgctacacgcagaacatagataccctggagcgaatagccgggctggaacaggaggacttggtggaggcgcacggcaccttctacacatcacactgcgtcagcgccagctgccggcacgaatacccgctaagctggatgaaagagaagatcttctctgaggtgacgcccaagtgtgaagactgtcagagcctggtgaagcctgatatcgtcttttttggtgagagcctcccagcgcgtttcttctcctgtatgcagtcagacttcctgaaggtggacctcctcctggtcatgggtacctccttgcaggtgcagccctttgcctccctcatcagcaaggcacccctctccacccctcgcctgctcatcaacaaggagaaagctggccagtcggaccctttcctggggatgattatgggcctcggaggaggcatggactttgactccaagaaggcctacagggacgtggcctggctgggtgaatgcgaccagggctgcctggcccttgctgagctccttggatggaagaaggagctggaggaccttgtccggagggagcacgccagcatagatgcccagtcgggggcgggggtccccaaccccagcacttcagcttcccccaagaagtccccgccacctgccaaggacgaggccaggacaacagagagggagaaaccccagtgacagctgcatctcccaggcgggatgccgagctcctcagggacagctgagccccaaccgggcctggccccctcttaaccagcagttcttgtctggggagctcagaacatcccccaatctcttacagctccctccccaaaactggggtcccagcaaccctggcccccaaccccagcaaatctctaacacctcctagaggccaaggcttaaacaggcatctctaccagccccactgtctctaaccactcctgggctaaggagtaacctccctcatctctaactgcccccacggggccagggctaccccagaacttttaactcttccaggacagggagcttcgggcccccactctgtctcctgcccccgggggcctgtggctaagtaaaccatacctaacctaccccagtgtgggtgtgggcctctgaatataacccacacccagcgtagggggagtctgagccgggagggctcccgagtctctgccttcagctcccaaagtgggtggtgggcccccttcacgtgggacccacttcccatgctggatgggcagaagacattgcttattggagacaaattaaaaacaaaaacaactaac

SEQ ID NO: 105 - DNA, 1931 bp, Homo sapiens
cgaaagcgcgtctgcggccgcaatgtctgctgagagttgtagttctgtgccctatcacggccactcccatttctggtgccgtcacgggacagagcagtcggtgacaggacagagcagtcggtgacgggacacagtggttggtgacgggacagagcggtcggtgacagcctcaagggcttcagcaccgcgcccatggcagagccagaccgactcagattcagactctgagggaggagccgctggtggagaagcagacatggacttcctgcggaacttattctcccagacgctcagcctgggcagccagaaggagcgtctgctggacgagctgaccttggaaggggtggcccggtacatgcagagcgaacgctgtcgcagagtcatctgtttggtgggagctggaatctccacatccgcaggcatccccgactttcgctctccatccaccggcctctatgacaacctagagaagtaccatcttccctacccagaggccatctttgagatcagctatttcaagaaacatccggaacccttcttcgccctcgccaaggaactctatcctgggcagttcaagccaaccatctgtcactacttcatgcgcctgctgaaggacaaggggctactcctgcgctgctacacgcagaacatagataccctggagcgaatagccgggctggaacaggaggacttggtggaggcgcacggcaccttctacacatcacactgcgtcagcgccagctgccggcacgaatacccgctaagctggatgaaagagaagatcttctctgaggtgacgcccaagtgtgaagactgtcagagcctggtgaagcctgatatcgtcttttttggtgagagcctcccagcgcgtttcttctcctgtatgcagtcagacttcctgaaggtggacctcctcctggtcatgggtacctccttgcaggtgcagccctttgcctccctcatcagcaaggcacccctctccacccctcgcctgctcatcaacaaggagaaagctggccagtcggaccctttcctggggatgattatgggcctcggaggaggcatggactttgactccaagaaggcctacagggacgtggcctggctgggtgaatgcgaccagggctgcctggcccttgctgagctccttggatggaagaaggagctggaggaccttgtccggagggagcacgccagcatagatgcccagtcgggggcgggggtccccaaccccagcacttcagcttcccccaagaagtccccgccacctgccaaggacgaggccaggacaacagagagggagaaaccccagtgacagctgcatctcccaggcgggatgccgagctcctcagggacagctgagccccaaccgggcctggccccctcttaaccagcagttcttgtctggggagctcagaacatcccccaatctcttacagctccctccccaaaactggggtcccagcaaccctggcccccaaccccagcaaatctctaacacctcctagaggccaaggcttaaacaggcatctctaccagccccactgtctctaaccactcctgggctaaggagtaacctccctcatctctaactgcccccacggggccagggctaccccagaacttttaactcttccaggacagggagcttcgggcccccactctgtctcctgcccccgggggcctgtggctaagtaaaccatacctaacctaccccagtgtgggtgtgggcctctgaatataacccacacccagcgtagggggagtctgagccgggagggctcccgagtctctgccttcagctcccaaagtgggtggtgggcccccttcacgtgggacccacttcccatgctggatgggcagaagacattgcttattggagacaaattaaaaacaaaaacaactaacaaaaaaaaaaaaaaaaaaaaaaa

SEQ ID NO: 106 - DNA, 1927 bp, Homo sapiens
ggcgccgggggcgggggtgggaggcggaggcggggccggggcgccgcgggcggggcgccgggggcggggcgagtccggaggactcctcggactgcgcggaacatggcgttctggggttggcgcgccgcggcagccctccggctgtggggccgggtagttgaacgggtcgaggccgggggaggcgtggggccgtttcaggcctgcggctgtcggctggtgcttggcggcagggacgatgtgagtgcggggctgagaggcagccatggggcccgcggtgagcccttggacccggcgcgccccttgcagaggcctcccagacccgaggtgcccagggcattccggaggcagccgagggcagcagctcccagtttcttcttttcgagtattaaaggtggaagaaggtccatatctttttctgtgggtgcttcaagtgttgttggaagtggaggcagcagtgacaaggggaagctttccctgcaggatgtagctgagctgattcgggccagagcctgccagagggtggtggtcatggtgggggccggcatcagcacacccagtggcattccagacttcagatcgccggggagtggcctgtacagcaacctccagcagtacgatctcccgtaccccgaggccatttttgaactcccattcttctttcacaaccccaagccctttttcactttggccaaggagctgtaccctggaaactacaagcccaacgtcactcactactttctccggctgcttcatgacaaggggctgcttctgcggctctacacgcagaacatcgatgggcttgagagagtgtcgggcatccctgcctcaaagctggttgaagctcatggaacctttgcctctgccacctgcacagtctgccaaagacccttcccaggggaggacattcgggctgacgtgatggcagacagggttccccgctgcccggtctgcaccggcgttgtgaagcccgacattgtgttctttggggagccgctgccccagaggttcttgctgcatgtggttgatttccccatggcagatctgctgctcatccttgggacctccctggaggtggagccttttgccagcttgaccgaggccgtgcggagctcagttccccgactgctcatcaaccgggacttggtggggcccttggcttggcatcctcgcagcagggacgtggcccagctgggggacgtggttcacggcgtggaaagcctagtggagcttctgggctggacagaagagatgcgggaccttgtgcagcgggaaactgggaagcttgatggaccagacaaataggatgatggctgcccccacacaataaatggtaacataggagacatccacatcccaattctgacaagacctcatgcctgaagacagcttgggcaggtgaaaccagaatatgtgaactgagtggacacccgaggctgccactggaatgtcttctcaggccatgagctgcagtgactggtagggctgtgtttacagtcagggccaccccgtcacatatacaaaggagctgcctgcctgtttgctgtgttgaactcttcactctgctgaagctcctaatggaaaaagctttcttctgactgtgaccctcttgaactgaatcagaccaactggaatcccagaccgagtctgctttctgtgcctagttgaacggcaagctcggcatctgttggttacaagatccagacttgggccgagcggtccccagccctcttcatgttccgaagtgtagtcttgaggccctggtgccgcacttctagcatgttggtctcctttagtggggctatttttaatgagagaaaatctgttctttccagcatgaaatacatttagtctcctcaaagggaaaaaaaaaaaaaaaaaaaaaaaaaaaaaaaaaaaaaaaaaaaaaaaaaaaaaaaaaaaaaaaaaa

SEQ ID NO: 107 - DNA, 1174 bp, Homo sapiens
gtccgtagagctgtgagagaatgaagatgagctttgcgttgactttcaggtcagcaaaaggccgttggatcgcaaaccccagccagccgtgctcgaaagcctccattgggttatttgtgccagcaagtcctcctctggaccctgagaaggtcaaagagttacagcgcttcatcaccctttccaagagactccttgtgatgactggggcaggaatctccaccgaatcggggataccagactacaggtcagaaaaagtggggctttatgcccgcactgaccgcaggcccatccagcatggtgattttgtccggagtgccccaatccgccagcggtactgggcgagaaacttcgtaggctggcctcaattctcctcccaccagcctaaccctgcacactgggctttgagcacctgggagaaactcggaaagctgtactggttggtgacccaaaatgtggatgctttgcacaccaaggcggggagtcggcgcctgacagagctccacggatgcatggacagggtcctgtgcttggattgtggggaacagactccccggggggtgctgcaagagcgtttccaagtcctgaaccccacctggagtgctgaggcccatggcctggctcctgatggtgacgtctttctctcagaggagcaagtccggagctttcaggtcccaacctgcgttcaatgtggaggccatctgaaaccagatgtcgttttcttcggggacacagtgaaccctgacaaggttgattttgtgcacaagcgtgtaaaagaagccgactccctcttggtggtgggatcatccttgcaggtatactctggttacaggtttatcctcactgcctgggagaagaagctcccgattgcaatactgaacattgggcccacacggtcggatgacttggcgtgtctgaaactgaattctcgttgtggagagttgctgcctttgatagacccatgctgaccacagcctgatattccagaacctggaacagggactttcacttgaatcttgctgctaaatgtaaatgccttctcaaatgacagattccagttcccattcaacagagtagggtgcactgacaaagtatagaaggttctaggtatcttaatgtgtggatattcttaattaaaactcattttttttaaataaaaaattgttcagctttaaaa

SEQ ID NO: 108 - DNA, 1670 bp, Homo sapiens
ccggagcgcggtcgggacacagcgcctctaggagaaagcctggaaggcgctccgggggtacccagagctcttagcgggccggcagcatgtgcggggcccaagtaaatggaaatgttttctaacatataaaaacctacagaagaagaaaataattttctggatcaaattagaagtctgtattatattgatgtctccagattcaaatatattagaaagcagccgtggagacaaccatcttcattttgggagaaataactaaagcccgcctcaagcattagaactacagacaaaccctgatgcgacctctccagattgtcccaagtcgattgatttcccagctatattgtggcctgaagcctccagcgtccacacgaaaccagatttgcctgaaaatggctcggccaagttcaagtatggcagattttcgaaagttttttgcaaaagcaaagcacatagtcatcatctcaggagctggtgttagtgcagaaagtggtgttccgaccttcagaggagctggaggttattggagaaaatggcaagcccaggacctggcgactcccctggcctttgcccacaacccgtcccgggtgtgggagttctaccactaccggcgggaggtcatggggagcaaggagcccaacgccgggcaccgcgccatagccgagtgtgagacccggctgggcaagcagggccggcgagtcgtggtcatcacccagaacatcgatgagctgcaccgcaaggctggcaccaagaaccttctggagatccatggtagcttatttaaaactcgatgtacctcttgtggagttgtggctgagaattacaagagtccaatttgtccagctttatcaggaaaaggtgctccagaacctggaactcaagatgccagcatcccagttgagaaacttccccggtgtgaagaggcaggctgcgggggcttgctgcgacctcacgtcgtgtggtttggagaaaacctggatcctgccattctggaggaggttgacagagagctcgcccactgtgatttatgtctagtggtgggcacttcctctgtggtgtacccagcagccatgtttgccccccaggtggctgccaggggcgtgccagtggctgaatttaacacggagaccaccccagctacgaacagattcaggtttcatttccagggaccctgtggaacgactcttcctgaagcccttgcctgtcatgaaaatgaaactgtttcttaagtgtcctggggaagaaagaaattacagtatatctaagaactaggccacacgcagaggagaaatggtcttatgggtggtgagctgagtactgaacaatctaaaaatagcctctgattccctcgctggaatccaacctgttgataagtgatgggggtttagaagtagcaaagagcacccacattcaaaagtcacagaactggaaagttaattcatattatttggtttgaactgaaacgtgaggtatctttgatgtgtatggttggttattgggagggaaaaattttgtaaattagattgtctaaaaaaaatagttattctgattatatttttgttatctgggcaaagtagaagtcaaggggtaaaaaccctactattctgatttttgcacaagttttagtggaaaataaaatcacactctacagtaaaaaaaaaaaaaaaaaa

SEQ ID NO: 109 - DNA, 2350 bp, Homo sapiens
attcgggggcgcgagctgccccagtaaatggaaatgttttctaacatataaaaacctacagaagaagaaaataattttctggatcaaattagaagtctgtattatattgatgtctccagattcaaatatattagaaagcagccgtggagacaaccatcttcattttgggcgaaataactaaagcccgcctcaagcattagaactacagacaaaccctgatgcgacctctccagattgtcccaagtcgattgatttcccagctatattgtggcctgaagcctccagcgtccacacgaaaccagatttgcctgaaaatggctcggccaagttcaagtatggcagattttcgaaagttttttgcaaaagcaaagcacatagtcatcatctcaggagctggtgttagtgcagaaagtggtgttccgaccttcagaggagctggaggttattggagaaaatggcaagcccaggacctggcgactcccctggcctttgcccacaacccgtcccgggtgtgggagttctaccactaccggcgggaggtcatggggagcaaggagcccaacgccgggcaccgcgccatagccgagtgtgagacccggctgggcaagcagggccggcgagtcgtggtcatcacccagaacatcgatgagctgcaccgcaaggctggcaccaagaaccttctggagatccatggtagcttatttaaaactcgatgtacctcttgtggagttgtggctgagaattacaagagtccaatttgtccagctttatcaggaaaaggtgctccagaacctggaactcaagatgccagcatcccagttgagaaacttccccggtgtgaagaggcaggctgcgggggcttgctgcgacctcacgtcgtgtggtttggagaaaacctggatcctgccattctggaggaggttgacagagagctcgcccactgtgatttatgtctagtggtgggcacttcctctgtggtgtacccagcagccatgtttgccccccaggtggctgccaggggcgtgccagtggctgaatttaacacggagaccaccccagctacgaacagattcagtcatttgatctccatctcatctctaattattataaagaattaaaacaagtcatcattgtagaaaagcaagaaaatgcagatagagaaaaagaagaaaataaaactggagtatttccacaacccaagtttagagttggcccccacctcccatgccatggactgagcagcaggggcccagcatcccttggatatggtggctgtgtcttcatgtgaaagaaactgaacttggtggtttttcctgccagttcaggagagattcttggcatgtaatatatatcactgctcaagtcaagcctcctaaaaccacagacctgtttcagctgctacttcagccaaaattcttcagcttcatattgtcttgaaaacctatgattgtctctaacaaacaggctacttgctagttagaaattcttatcaatttggcaagctacttatcaaccagactgaccacaagaactgtcatctcatcaatgaaggagtaactgatcaatgaagccagcaatgcttttttcttggcatcatcaaagctgacatttagaagagatgctggtgatagtcatctcatcctactcaatttttcaaaggcagaaaccaaccctggagcaattgagaggactgtttaaacacagagcttaacaatggcagaattgtatatctcgtgcttaacagattttggttgaactttaccctaggtcaggggtcagcaaactactgcctgtgggccaaatttgcccaccacctgtatctgtaaataaggtttcattggaacacagctgtggccatatgtttgtatattgtgtgtggctgcttttgcattaggatgacagaggtgaatagttgcaacagagactggctggtctgcaaagcctaaaatatgtcctgtgtggccctttacagaaaaagttttctaacccctgctctaggttacggagaaaaaaaaatggaataatgttctctgctacttttaacctgattttctttgtacctaaataggcagctagaatgctgcctatattttaataaggatttggatctcacaagacaccttaggcctacacaagttgttcagattctttgccccagttctaatctagtgacaaaggcatagaattctcctcccacaggaatgtatttctattttcaaggtgttaattagttccagttttggttttgtcgttttccccatgtccgatgcttatattggatgatttctgataaacctgactattccaataaaccctaggcatttttgaatttaaaaaaaaaaaaaaaaaa

SEQ ID NO: 110 - DNA, 1718 bp, Homo sapiens
gcggaagcggaagagcaggtctccaggggagcgatggcagccgggggtctgagccgctccgagcgcaaagcggcggagcgggtccggaggttgcgggaggagcagcagagggagcgcctccgccaggtgtcgcgcatcctgaggaaggcggcggcggagcgcagcgccgaggagggccggctgctggccgagagcgcggacctggtaacggagctgcagggccggagccggcggcgcgagggcctgaagcggcggcaggaggaggtgtgcgacgacccggaggagctgcgggggaaggtccgggagctggccagcgccgtccggaacgccaaatacttggtcgtctacacaggcgcgggaatcagcacggcagcgtctatcccagactaccggggccctaatggagtgtggacactgcttcagaaagggagaagcgttagtgctgccgacctgagcgaggccgagccaaccctcacccacatgagcatcacccgtctgcatgagcagaagctggtgcagcatgtggtgtctcagaactgtgacgggctccacctgaggagtgggctgccgcgcacggccatctccgagctccacgggaacatgtacattgaagtctgtacctcctgcgttcccaacagggagtacgtgcgggtgttcgatgtgacggagcgcactgccctccacagacaccagacaggccggacctgccacaagtgtgggacccagctgcgggacaccattgtgcactttggggagagggggacgttggggcagcctctgaactgggaagcggcgaccgaggctgccagcagagcagacaccatcctgtgtctagggtccagcctgaaggttctaaagaagtacccacgcctctggtgcatgaccaagccccctagccggcggccgaagctttacatcgtgaacctgcagtggaccccgaaggatgactgggctgccctgaagctacatgggaagtgtgatgacgtcatgcggctcctcatggccgagctgggcttggagatccccgcctatagcaggtggcaggatcccattttctcactggcgactcccctgcgtgctggtgaagaaggcagccacagtcggaagtcgctgtgcagaagcagagaggaggccccgcctggggaccggggtgcaccgcttagctcggcccccatcctagggggctggtttggcaggggctgcacaaaacgcacaaaaaggaagaaagtgacgtaatcacgtgctcgatgaagaacagttggcactttgcagatggccagtgtcacggtgaaggctgggttgcccccacgggtctagggagaacgaactctttggggatgacattttcaccgtgacatttttagccatttgtccttgaggaagccccttgcactgctgcggttgtaccctgatacggcctggccatcgaggacacctgcccatccggcctctgtgtcaagaggtggcagccgcacctttctgtgagaacggaactcgggttatttcagccccggcctgcagagtggaagcgcccagcggcctttcctcgctcaccaggccagtctcagggcctcaccgtatttctactactacttaatgaaaaagtgtgaactttatagaatcctctctgtactggatgtgcggcagaggggtggctccgagcctcggctctatgcagacctttttatttctattaaacgtttctgcactggcaaaaa

SEQ ID NO: 111 - DNA, 10182 bp, Homo sapiens
ccggaaaatggccgccgccgccgccgccgcgccgagcggaggaggaggaggaggcgaggaggagagactgctccataaaaatacagactcaccagttcctgctttgatgtgacatgtgactccccagaatacaccttgcttctgtagaccagctccaacaggattccatggtagctgggatgttagggctcagggaagaaaagtcagaagaccaggacctccagggcctcaaggacaaacccctcaagtttaaaaaggtgaagaaagataagaaagaagagaaagagggcaagcatgagcccgtgcagccatcagcccaccactctgctgagcccgcagaggcaggcaaagcagagacatcagaagggtcaggctccgccccggctgtgccggaagcttctgcctcccccaaacagcggcgctccatcatccgtgaccggggacccatgtatgatgaccccaccctgcctgaaggctggacacggaagcttaagcaaaggaaatctggccgctctgctgggaagtatgatgtgtatttgatcaatccccagggaaaagcctttcgctctaaagtggagttgattgcgtacttcgaaaaggtaggcgacacatccctggaccctaatgattttgacttcacggtaactgggagagggagcccctcccggcgagagcagaaaccacctaagaagcccaaatctcccaaagctccaggaactggcagaggccggggacgccccaaagggagcggcaccacgagacccaaggcggccacgtcagagggtgtgcaggtgaaaagggtcctggagaaaagtcctgggaagctccttgtcaagatgccttttcaaacttcgccagggggcaaggctgaggggggtggggccaccacatccacccaggtcatggtgatcaaacgccccggcaggaagcgaaaagctgaggccgaccctcaggccattcccaagaaacggggccgaaagccggggagtgtggtggcagccgctgccgccgaggccaaaaagaaagccgtgaaggagtcttctatccgatctgtgcaggagaccgtactccccatcaagaagcgcaagacccgggagacggtcagcatcgaggtcaaggaagtggtgaagcccctgctggtgtccaccctcggtgagaagagcgggaaaggactgaagacctgtaagagccctgggcggaaaagcaaggagagcagccccaaggggcgcagcagcagcgcctcctcaccccccaagaaggagcaccaccaccatcaccaccactcagagtccccaaaggcccccgtgccactgctcccacccctgcccccacctccacctgagcccgagagctccgaggaccccaccagcccccctgagccccaggacttgagcagcagcgtctgcaaagaggagaagatgcccagaggaggctcactggagagcgacggctgccccaaggagccagctaagactcagcccgcggttgccaccgccgccacggccgcagaaaagtacaaacaccgaggggagggagagcgcaaagacattgtttcatcctccatgccaaggccaaacagagaggagcctgtggacagccggacgcccgtgaccgagagagttagctgactttacacggagcggattgcaaagcaaaccaacaagaataaaggcagctgttgtctcttctccttatgggtagggctctgacaaagcttcccgattaactgaaataaaaaatatttttttttctttcagtaaacttagagtttcgtggcttcagggtgggagtagttggagcattggggatgtttttcttaccgacaagcacagtcaggttgaagacctaaccagggccagaagtagctttgcacttttctaaactaggctccttcaacaaggcttgctgcagatactactgaccagacaagctgttgaccaggcacctcccctcccgcccaaacctttcccccatgtggtcgttagagacagagcgacagagcagttgagaggacactcccgttttcggtgccatcagtgccccgtctacagctcccccagctccccccacctcccccactcccaaccacgttgggacagggaggtgtgaggcaggagagacagttggattctttagagaagatggatatgaccagtggctatggcctgtgcgatcccacccgtggtggctcaagtctggccccacaccagccccaatccaaaactggcaaggacgcttcacaggacaggaaagtggcacctgtctgctccagctctggcatggctaggaggggggagtcccttgaactactgggtgtagactggcctgaaccacaggagaggatggcccagggtgaggtggcatggtccattctcaagggacgtcctccaacgggtggcgctagaggccatggaggcagtaggacaaggtgcaggcaggctggcctggggtcaggccgggcagagcacagcggggtgagagggattcctaatcactcagagcagtctgtgacttagtggacaggggagggggcaaagggggaggagaagaaaatgttcttccagttactttccaattctcctttagggacagcttagaattatttgcactattgagtcttcatgttcccacttcaaaacaaacagatgctctgagagcaaactggcttgaattggtgacatttagtccctcaagccaccagatgtgacagtgttgagaactacctggatttgtatatatacctgcgcttgttttaaagtgggctcagcacatagggttcccacgaagctccgaaactctaagtgtttgctgcaattttataaggacttcctgattggtttctcttctccccttccatttctgccttttgttcatttcatcctttcacttctttcccttcctccgtcctcctccttcctagttcatcccttctcttccaggcagccgcggtgcccaaccacacttgtcggctccagtccccagaactctgcctgccctttgtcctcctgctgccagtaccagccccaccctgttttgagccctgaggaggccttgggctctgctgagtccgacctggcctgtctgtgaagagcaagagagcagcaaggtcttgctctcctaggtagccccctcttccctggtaagaaaaagcaaaaggcatttcccaccctgaacaacgagccttttcacccttctactctagagaagtggactggaggagctgggcccgatttggtagttgaggaaagcacagaggcctcctgtggcctgccagtcatcgagtggcccaacaggggctccatgccagccgaccttgacctcactcagaagtccagagtctagcgtagtgcagcagggcagtagcggtaccaatgcagaactcccaagacccgagctgggaccagtacctgggtccccagcccttcctctgctcccccttttccctcggagttcttcttgaatggcaatgttttgcttttgctcgatgcagacagggggccagaacaccacacatttcactgtctgtctggtccatagctgtggtgtaggggcttagaggcatgggcttgctgtgggtttttaattgatcagttttcatgtgggatcccatctttttaacctctgttcaggaagtccttatctagctgcatatcttcatcatattggtatatccttttctgtgtttacagagatgtctcttatatctaaatctgtccaactgagaagtaccttatcaaagtagcaaatgagacagcagtcttatgcttccagaaacacccacaggcatgtcccatgtgagctgctgccatgaactgtcaagtgtgtgttgtcttgtgtatttcagttattgtccctggcttccttactatggtgtaatcatgaaggagtgaaacatcatagaaactgtctagcacttccttgccagtctttagtgatcaggaaccatagttgacagttccaatcagtagcttaagaaaaaaccgtgtttgtctcttctggaatggttagaagtgagggagtttgccccgttctgtttgtagagtctcatagttggactttctagcatatatgtgtccatttccttatgctgtaaaagcaagtcctgcaaccaaactcccatcagcccaatccctgatccctgatcccttccacctgctctgctgatgacccccccagcttcacttctgactcttccccaggaagggaaggggggtcagaagagagggtgagtcctccagaactcttcctccaaggacagaaggctcctgcccccatagtggcctcgaactcctggcactaccaaaggacacttatccacgagagcgcagcatccgaccaggttgtcactgagaagatgtttattttggtcagttgggtttttatgtattatacttagtcaaatgtaatgtggcttctggaatcattgtccagagctgcttccccgtcacctgggcgtcatctggtcctggtaagaggagtgcgtggcccaccaggcccccctgtcacccatgacagttcattcagggccgatggggcagtcgtggttgggaacacagcatttcaagcgtcactttatttcattcgggccccacctgcagctccctcaaagaggcagttgcccagcctctttcccttccagtttattccagagctgccagtggggcctgaggctccttagggttttctctctatttccccctttcttcctcattccctcgtctttcccaaaggcatcacgagtcagtcgcctttcagcaggcagccttggcggtttatcgccctggcaggcaggggccctgcagctctcatgctgcccctgccttggggtcaggttgacaggaggttggagggaaagccttaagctgcaggattctcaccagctgtgtccggcccagttttggggtgtgacctcaatttcaattttgtctgtacttgaacattatgaagatgggggcctctttcagtgaatttgtgaacagcagaattgaccgacagctttccagtacccatggggctaggtcattaaggccacatccacagtctcccccacccttgttccagttgttagttactacctcctctcctgacaatactgtatgtcgtcgagctccccccaggtctacccctcccggccctgcctgctggtgggcttgtcatagccagtgggattgccggtcttgacagctcagtgagctggagatacttggtcacagccaggcgctagcacagctcccttctgttgatgctgtattcccatatcaaaagacacaggggacacccagaaacgccacatcccccaatccatcagtgccaaactagccaacggccccagcttctcagctcgctggatggcggaagctgctactcgtgagcgccagtgcgggtgcagacaatcttctgttgggtggcatcattccaggcccgaagcatgaacagtgcacctgggacagggagcagccccaaattgtcacctgcttctctgcccagcttttcattgctgtgacagtgatggcgaaagagggtaataaccagacacaaactgccaagttgggtggagaaaggagtttctttagctgacagaatctctgaattttaaatcacttagtaagcggctcaagcccaggagggagcagagggatacgagcggagtcccctgcgcgggaccatctggaattggtttagcccaagtggagcctgacagccagaactctgtgtcccccgtctaaccacagctccttttccagagcattccagtcaggctctctgggctgactgggccaggggaggttacaggtaccagttctttaagaagatctttgggcatatacatttttagcctgtgtcattgccccaaatggattcctgtttcaagttcacacctgcagattctaggacctgtgtcctagacttcagggagtcagctgtttctagagttcctaccatggagtgggtctggaggacctgcccggtgggggggcagagccctgctccctccgggtcttcctactcttctctctgctctgacgggatttgttgattctctccattttggtgtctttctcttttagatattgtatcaatctttagaaaaggcatagtctacttgttataaatcgttaggatactgcctcccccagggtctaaaattacatattagaggggaaaagctgaacactgaagtcagttctcaacaatttagaaggaaaacctagaaaacatttggcagaaaattacatttcgatgtttttgaatgaatacgagcaagcttttacaacagtgctgatctaaaaatacttagcacttggcctgagatgcctggtgagcattacaggcaaggggaatctggaggtagccgacctgaggacatggcttctgaacctgtcttttgggagtggtatggaaggtggagcgttcaccagtgacctggaaggcccagcaccaccctccttcccactcttctcatcttgacagagcctgccccagcgctgacgtgtcaggaaaacacccagggaactaggaaggcacttctgcctgaggggcagcctgccttgcccactcctgctctgctcgcctcggatcagctgagccttctgagctggcctctcactgcctccccaaggccccctgcctgccctgtcaggaggcagaaggaagcaggtgtgagggcagtgcaaggagggagcacaacccccagctcccgctccgggctccgacttgtgcacaggcagagcccagaccctggaggaaatcctacctttgaattcaagaacatttggggaatttggaaatctctttgcccccaaacccccattctgtcctacctttaatcaggtcctgctcagcagtgagagcagatgaggtgaaaaggccaagaggtttggctcctgcccactgatagcccctctccccgcagtgtttgtgtgtcaagtggcaaagctgttcttcctggtgaccctgattatatccagtaacacatagactgtgcgcataggcctgctttgtctcctctatcctgggcttttgttttgctttttagttttgcttttagtttttctgtcccttttatttaacgcaccgactagacacacaaagcagttgaatttttatatatatatctgtatattgcacaattataaactcattttgcttgtggctccacacacacaaaaaaagacctgttaaaattatacctgttgcttaattacaatatttctgataaccatagcataggacaagggaaaataaaaaaagaaaaaaaagaaaaaaaaacgacaaatctgtctgctggtcacttcttctgtccaagcagattcgtggtcttttcctcgcttctttcaagggctttcctgtgccaggtgaaggaggctccaggcagcacccaggttttgcactcttgtttctcccgtgcttgtgaaagaggtcccaaggttctgggtgcaggagcgctcccttgacctgctgaagtccggaacgtagtcggcacagcctggtcgccttccacctctgggagctggagtccactggggtggcctgactcccccagtccccttcccgtgacctggtcagggtgagcccatgtggagtcagcctcgcaggcctccctgccagtagggtccgagtgtgtttcatccttcccactctgtcgagcctgggggctggagcggagacgggaggcctggcctgtctcggaacctgtgagctgcaccaggtagaacgccagggaccccagaatcatgtgcgtcagtccaaggggtcccctccaggagtagtgaagactccagaaatgtccctttcttctcccccatcctacgagtaattgcatttgcttttgtaattcttaatgagcaatatctgctagagagtttagctgtaacagttctttttgatcatctttttttaataattagaaacaccaaaaaaatccagaaacttgttcttccaaagcagagagcattataatcaccagggccaaaagcttccctccctgctgtcattgcttcttctgaggcctgaatccaaaagaaaaacagccataggccctttcagtggccgggctacccgtgagcccttcggaggaccagggctggggcagcctctgggcccacatccggggccagctccggcgtgtgttcagtgttagcagtgggtcatgatgctctttcccacccagcctgggataggggcagaggaggcgaggaggccgttgccgctgatgtttggccgtgaacaggtgggtgtctgcgtgcgtccacgtgcgtgttttctgactgacatgaaatcgacgcccgagttagcctcacccggtgacctctagccctgcccggatggagcggggcccacccggttcagtgtttctggggagctggacagtggagtgcaaaaggcttgcagaacttgaagcctgctccttcccttgctaccacggcctcctttccgtttgatttgtcactgcttcaatcaataacagccgctccagagtcagtagtcaatgaatatatgaccaaatatcaccaggactgttactcaatgtgtgccgagcccttgcccatgctgggctcccgtgtatctggacactgtaacgtgtgctgtgtttgctccccttccccttccttctttgccctttacttgtctttctggggtttttctgtttgggtttggtttggtttttatttctccttttgtgttccaaacatgaggttctctctactggtcctcttaactgtggtgttgaggcttatatttgtgtaatttttggtgggtgaaaggaattttgctaagtaaatctcttctgtgtttgaactgaagtctgtattgtaactatgtttaaagtaattgttccagagacaaatatttctagacactttttctttacaaacaaaagcattcggagggagggggatggtgactgagatgagaggggagagctgaacagatgacccctgcccagatcagccagaagccacccaaagcagtggagcccaggagtcccactccaagccagcaagccgaatagctgatgtgttgccactttccaagtcactgcaaaaccaggttttgttccgcccagtggattcttgttttgcttcccctccccccgagattattaccaccatcccgtgcttttaaggaaaggcaagattgatgtttccttgaggggagccaggaggggatgtgtgtgtgcagagctgaagagctggggagaatggggctgggcccacccaagcaggaggctgggacgctctgctgtgggcacaggtcaggctaatgttggcagatgcagctcttcctggacaggccaggtggtgggcattctctctccaaggtgtgccccgtgggcattactgtttaagacacttccgtcacatcccaccccatcctccagggctcaacactgtgacatctctattccccaccctccccttcccagggcaataaaatgaccatggagggggcttgcactctcttggctgtcacccgatcgccagcaaaacttagatgtgagaaaaccccttcccattccatggcgaaaacatctccttagaaaagccattaccctcattaggcatggttttgggctcccaaaacacctgacagcccctccctcctctgagaggcggagagtgctgactgtagtgaccattgcatgccgggtgcagcatctggaagagctaggcagggtgtctgccccctcctgagttgaagtcatgctcccctgtgccagcccagaggccgagagctatggacagcattgccagtaacacaggccaccctgtgcagaagggagctggctccagcctggaaacctgtctgaggttgggagaggtgcacttggggcacagggagaggccgggacacacttagctggagatgtctctaaaagccctgtatcgtattcaccttcagtttttgtgttttgggacaattactttagaaaataagtaggtcgttttaaaaacaaaaattattgattgcttttttgtagtgttcagaaaaaaggttctttgtgtatagccaaatgactgaaagcactgatatatttaaaaacaaaaggcaatttattaaggaaatttgtaccatttcagtaaacctgtctgaatgtacctgtatacgtttcaaaaacaccccccccccactgaatccctgtaacctatttattatataaagagtttgccttataaattt

SEQ ID NO: 112 - DNA, 2170 bp, Homo sapiens
caggaagcccacccagccccgccacgcagagcccagaaggaaagaaagcctcatgcctgagccgaggggagcaccatggatctgacaaaaatgggcatgatccagctgcagaaccctagccaccccacggggctactgtgcaaggccaaccagatgcggctggccgggactttgtgcgatgtggtcatcatggtggacagccaggagttccacgcccaccggacggtgctggcctgcaccagcaagatgtttgagatcctcttccaccgcaatagtcaacactatactttggacttcctctcgccaaagaccttccagcagattctggagtatgcatatacagccacgctgcaagccaaggcggaggacctggatgacctgctgtatgcggccgagatcctggagatcgagtacctggaggaacagtgcctgaagatgctggagaccatccaggcctcagacgacaatgacacggaggccaccatggccgatggcggggccgaggaagaagaggaccgcaaggctcggtacctcaagaacatcttcatctcgaagcattccagcgaggagagtgggtatgccagtgtggctggacagagcctccctgggcccatggtggaccagagcccttcagtctccacttcatttggtctttcagccatgagtcccaccaaggctgcagtggacagtttgatgaccataggacagtctctcctgcagggaactcttcagccacctgcagggcccgaggagccaactctggctgggggtgggcggcaccctggggtggctgaggtgaagacggagatgatgcaggtggatgaggtgcccagccaggacagccctggggcagccgagtccagcatctcaggagggatgggggacaaggttgaggaaagaggcaaagaggggcctgggaccccgactcgaagcagcgtcatcaccagtgctagggagctacactatgggcgagaggagagtgccgagcaggtgccacccccagctgaggctggccaggcccccactggccgacctgagcacccagcacccccgcctgagaagcatctgggcatctactccgtgttgcccaaccacaaggctgacgctgtattgagcatgccgtcttccgtgacctctggcctccacgtgcagcctgccctggctgtctccatggacttcagcacctatggggggctgctgccccagggcttcatccagagggagctgttcagcaagctgggggagctggctgtgggcatgaagtcagagagccggaccatcggagagcagtgcagcgtgtgtggggtcgagcttcctgataacgaggctgtggagcagcacaggaagctgcacagtgggatgaagacgtacgggtgcgagctctgcgggaagcggttcctggatagtttgcggctgagaatgcacttactggctcattcagcgggtgccaaagcctttgtctgtgatcagtgcggtgcacagttttcgaaggaggatgccctggagacacacaggcagacccatactggcactgacatggccgtcttctgtctgctgtgtgggaagcgcttccaggcgcagagcgcactgcagcagcacatggaggtccacgcgggcgtgcgcagctacatctgcagtgagtgcaaccgcaccttccccagccacacggctctcaaacgccacctgcgctcacatacaggcgaccacccctacgagtgtgagttctgtggcagctgcttccgggatgagagcacactcaagagccacaaacgcatccacacgggtgagaaaccctacgagtgcaatggctgtgacaagaagttcagcctcaagcatcagctggagacgcactatagggtgcacacaggtgagaagccctttgagtgtaagctctgccaccagcgctcccgggactactcggccatgatcaagcacctgagaacgcacaacggcgcctcgccctaccagtgcaccatctgcacagagtactgccccagcctctcctccatgcagaagcacatgaagggccacaagcccgaggagatcccgcccgactggaggatagagaagacgtacctctacctgtgctatgtgtgaagggaggcccgcggcggtggagccgagcggggagccaggaaagaagagttggagtgagatgaaggaaggacta

SEQ ID NO: 113 - DNA, 1440 bp, Homo sapiens
ggaattccgtagctattgatttcccggatctggtaacatggcaaaagatgccggtctaattgaagccaacggagaactcaaggtcttcatagaccagaaccttagtcccgggaaaggcgtggtgtccctcgtggccgttcacccctccaccgtcaacccgctcgggaagcagctcttgccaaaaacctttggacagtccaatgtcaacattgcccagcaagtggtaattggtacgcctcagagaccggcagcgtcaaacaccctggtggtaggaagcccacacacccccagcactcactttgcctctcagaaccagccttccgactcctcaccttggtctgccgggaagcgcaacaggaaaggagagaagaatggcaagggcctacggcatttctccatgaaggtctgcgagaaggtgcagaggaaagggaccacttcctacaacgaagtggcagacgagctggttgcggagttcagtgctgccgacaaccacatcttaccaaacgagtcagcttatgaccagaaaaacataagacggcgcgtctacgatgccttaaacgtgctaatggccatgaacatcatctccaaggagaagaaggagatcaagtggattggtctgcccaccaactcggctcaggaatgtcagaacttagaggtggaaagacagaggagacttgaaagaataaaacagaaacagtctcaacttcaagaacttattctacagcaaattgccttcaagaacctggtgcagagaaaccggcatgcggagcagcaggccagccggccaccgccacccaactcagtcatccacctgcccttcatcatcgtcaacaccagcaagaagacggtcatcgactgcagcatctccaatgacaaatttgagtatctgtttaattttgacaacacatttgaaatccacgatgacatagaagtgctgaagcggatgggcatggcttgcgggctggagtcggggagctgctctgccgaagaccttaaaatggccagaagtctggtccccaaggctctggagccatacgtgacagaaatggctcagggaactgttggaggcgtgttcatcacgacggcaggttccacgtctaacggcacaaggttctctgccagtgacctgaccaacggtgcagatgggatgctggccacaagctccaatgggtctcagtacagcggctccagggtggagactccggtgtcctacgtcggggaggacgacgaggaggacgatgacttcaacgagaatgacgaggacgactgacgtcctccccacttcagattcggcttcaggaaaacgtttagcgaaaagaaactttttttttaatgtgggttttctgtttccttttggcctagtcccaagaagatattggtaagctattgaatttagatatgcacctctgataagcaaggattgtttcccgtagattagga

SEQ ID NO: 114 - DNA, 1125 bp, Homo sapiens
cccccatgtgacagtgacggggtccccgctccaggagacgctcgagtctgcgtcccggccctcagcactgtccactgtttcggtgccagcagagaccagcaggcccgggacagttggtgtttggccgtgccgctgtctaacttggtgtgcagagtgaattgccgctgccggagcggagagaggcggagcggccaggagagaggggatttctgtcagcgccggcctcgggagctcggagacatgaacggcttcacgcctgacgagatgagccgcggcggggatgcggccgccgcagtggccgcagtggtcgctgccgcggccgccgccgcctcggcggggaacgggaccggcgcgggcaccggggctgaggtgccgggcgcgggggcggtctcagcggctgggcccccgggggcggccgggccgggccccgggcaactgtgctgcctgcgggaggatggtgagcggtgcggccgggcggcaggcaacgccagcttcagcaagaggatccagaagagcatctcccagaagaaggtgaagatcgagctggataagagcgcaaggcatctttacatatgtgattatcataaaaacttaattcagagtgttcgaaacagaagaaagagaaaagggagtgatgatgatggaggtgattcacctgttcaagatattgataccccagaggttgatttataccaattacaagtaaatacacttaggagatacaaaagacacttcaagctaccaaccagaccaggacttaataaagcacaacttgttgagatagttggttgccactttaggtctattccagtgaatgaaaaagacaccttaacatatttcatctactcagtgaagaatgacaagaacaaatcagatctcaaggttgatagtggtgttcactaggagacgtggaattgagactaataacttggatgttaacactgtttactgttttttcacatgtagaaatgttctttgtgtattttttctacagaggattttctctgattttattttctttgtttctgactctaataattagttggaaactcatataaaatgagctttcctaaattaaatctattttaaataaaggttattactattaaaaaaaaaaaaaaaaaa

SEQ ID NO: 115 - DNA, 1293 bp, Homo sapiens
aggtttgaactgactttattattttgtaaatgtgaattttacaaagcgctttacaattaatgatcacatccttttgtttgtcatggatttccactgtctgaaacggctctgagcacgcttgaagccctcggtttccctgttcgcttttgaatgtttcagttttagttattgatacaatgtcagccatggctaaaaagtaacagtcttgactctaccgagtaacagcacaaaaacagagtgagggctcaggaaaacaaaacaaaggcttcctccttaaaaaaaagacaaaaaaaaaaagctaagcatctgtggctgaaatctaactcagtggtactgtgaaaccttcctttacagcacaggaaaatttattttttaacagtcgtgagttacagtactttaacccctaaacagactctttaaaacaaccgtctcctttttaaaagtctcttttttccaaacattccatccgaaggatggatgctctacttgcacccagtgccatccaaatgttcaagtcaaaaatatttatacattttatacttaggcgagcgtctcgcaggccgtaggaggaagatggcggtggagtcgcgcgttacccaggaggaaattaagaaggagccagagaaaccgatcgaccgcgagaagacatgcccactgttgctacgggtcttcaccaccaataacggccgccaccaccgaatggacgagttctcccggggaaatgtaccgtccagcgagttgcagatctacacttggatggatgcaactttgaaagaactgacaagcttagtaaaagaagtctacccagaagctagaaagaagggcactcacttcaattttgcaatcgtttttacagatgttaaaagacctggctatcgagttaaggagattggcagcaccatgtctggcagaaaggggactgatgattccatgaccctgcagtcgcagaagttccagataggagattacttggacatagcaattacccctccaaatcgggcaccacctccttcagggcgcatgagaccatattaaattctatttactatttgttgaatttatttttccgtcagttatgtaaaataaacatactcttcttcctcccctgattattgccattaagcctttaaattctaaacaaattataatgcatcatctatttaggagttagatttggatgtgctattgtatgattacgaatagtctgtatgtttcaagcccttctgtaaaatatgaagaaaagtctcttagcattctgtgtaaaactgtactgttaaatatatgtgtgtaatc

SEQ ID NO: 116 - DNA, 1946 bp, Homo sapiens
gcctcgtcagctgcctgggcgggctgggaggcgcgggttgaaaagtctcgttccaagtttggagagagagagaagagcgcctcagacctcggtacccgcgagcggggaggaggcaggaaagaaggacgcggcgtctggggagcacccaggcagcaagacggggcccgggctttcgacagtggggagtgtgacgcgcttgggaaaggcaggagcgccagcggtcgggctgctcttggctaacgagaggagtccgaggcggcggcgaggggcgaacgacccgacgcaagatggcgagtaaagagatgtttgaagatactgtggaggagcgtgtcatcaatgaagaatataaaatctggaagaagaatacaccgtttctatatgacctggttatgacccatgctcttcagtggcccagtcttaccgttcagtggcttcctgaagtgactaaacctgaaggaaaagattatgcccttcattggctagtgctggggactcatacgtctgatgagcagaatcatctggtggttgctcgagtacatattcccaatgatgatgcacagtttgatgcttcccattgtgacagtgacaagggtgaatttggtggctttggttctgtaacaggaaaaattgaatgtgaaattaaaatcaatcacgaaggagaagtaaaccgtgctcgttacatgccgcagaatcctcacatcattgctacaaaaacaccatcttctgatgtgttggtttttgactatacaaaacaccctgctaaaccagacccaagtggagaatgtaatcctgatctcagattaagaggtcaccagaaggaaggctatggtctctcctggaattcaaatttgagtggacatctcctaagtgcatctgatgaccatactgtttgtctgtgggatataaacgcaggaccaaaagaaggcaaaattgtggatgctaaagccatctttactggccactcagctgttgtagaggatgtggcctggcacctgctgcacgagtcattgtttggatctgttgctgatgatcagaaacttatgatatgggacaccaggtccaataccacctccaagccgagtcacttggtggatgcgcacactgccgaagtcaactgcctctcattcaatccctacagcgaatttattctagccaccggctctgcggataagaccgtagctttatgggatctgcgtaacttaaaattaaaactccataccttcgaatctcataaagatgaaattttccaggtccactggtctccacataatgaaactattctggcttcaagtggtactgaccgccgcctgaatgtgtgggatttaagtaaaattggggaagaacaatcagcagaagatgcagaagatgggcctccagaactcctgtttattcatggaggacacactgctaagatttcagattttagctggaaccccaatgagccttgggtcatttgctcagtgtctgaggataacatcatgcagatatggcaaatggctgaaaatatttacaatgatgaagagtcagatgtcacgacatccgaactggagggacaaggatcttaaacccaaagtacgagaaatgtttctgttgaatgtaatgctacatgaatgcttgatttatcaagcgccaaaaaggcattgtatagtaggaaatgtaagtggggtggcttatggcttctttatcctctgattctagcactttcaagtgagctgttgcgtactgtatcatattgtagctattagggaagagaagaatgttgcttaagaaagaacatcaccattgattttaaatacaagtagcagggtattgcctttgattcaactgttttaagtcctcattttctcaaactaagtgcttgctgttcccaaatatgcaagaataacttttacactttttccttccaacacttcttgattggctttgcagaaataaagttttaaaat

SEQ ID NO: 117 - DNA, 2314 bp, Homo sapiens
cgcgcgcacagagcgagctcttgcagcctccccgcccctcccgcaacgctcgaccccaggattcccccggctcgcctgcccgccatggccgacaaggaagcagccttcgacgacgcagtggaagaacgagtgatcaacgaggaatacaaaatatggaaaaagaacaccccttttctttatgatttggtgatgacccatgctctggagtggcccagcctaactgcccagtggcttccagatgtaaccagaccagaagggaaagatttcagcattcatcgacttgtcctggggacacacacatcggatgaacaaaaccatcttgttatagccagtgtgcagctccctaatgatgatgctcagtttgatgcgtcacactacgacagtgagaaaggagaatttggaggttttggttcagttagtggaaaaattgaaatagaaatcaagatcaaccatgaaggagaagtaaacagggcccgttatatgccccagaacccttgtatcatcgcaacaaagactccttccagtgatgttcttgtctttgactatacaaaacatccttctaaaccagatccttctggagagtgcaacccagacttgcgtctccgtggacatcagaaggaaggctatgggctttcttggaacccaaatctcagtgggcacttacttagtgcttcagatgaccataccatctgcctgtgggacatcagtgccgttccaaaggagggaaaagtggtagatgcgaagaccatctttacagggcatacggcagtagtagaagatgtttcctggcatctactccatgagtctctgtttgggtcagttgctgatgatcagaaacttatgatttgggatactcgttcaaacaatacttccaaaccaagccactcagttgatgctcacactgctgaagtgaactgcctttctttcaatccttatagtgagttcattcttgccacaggatcagctgacaagactgttgccttgtgggatctgagaaatctgaaacttaagttgcattcctttgagtcacataaggatgaaatattccaggttcagtggtcacctcacaatgagactattttagcttccagtggtactgatcgcagactgaatgtctgggatttaagtaaaattggagaggaacaatccccagaagatgcagaagacgggccaccagagttgttgtttattcatggtggtcatactgccaagatatctgatttctcctggaatcccaatgaaccttgggtgatttgttctgtatcagaagacaatatcatgcaagtgtggcaaatggcagagaacatttataatgatgaagaccctgaaggaagcgtggatccagaaggacaagggtcctagatatgtctttacttgttgtgattttagactccccttttttcttctcaaccctgagagtgatttaacactggttttgagacagactttattcagctatccctctatataataggtaccaccgataatgctattagcccaaaccgtgggtttttctaaatattaataggggggcttgattcaacaaagccacagacttaacgttgaaattttcttcaggaattttctagtaacccaggtctaaagtagctacagaaaggggaatattatgtgtgattatttttcttcttatgctatatccccaagtttttcagactcatttaagtaaaggctagagtgagtaaggaatagagccaaatgaggtaggtgtctgagccatgaagtataaatactgaaagatgtcacttttattcaggaaatagggggagttcaagtcgtatagattcctactcgaaaatcttgacacctgactttccaggatgcacattttcatacgtagaccagtttcctcttggtttcttcagttaagtcaaaacaacacgttcctctttccccatatattcatatatttttgctcgttagtgtatttcttgagctgttttcatgttgtttatttcctgtctgtgaaatggtgtttttttttttgttgttggttttttttttttttttttaacttgggaccaccaagttgtaaagatgtatgtttttacctgacagttataccacaggtagactgtcaagttgagaagagtgaatcaataacttgtatttgttttaaaaattaaattaatccttgataagagttgctttttttttttaggagttagtccttgaccactagtttgatgccatctccattttgggtgacctgtttcaccagcaggcctgttactctccatgactaactgtgtaagtgcttaaaatggaataaattgcttttctacataaaaaaaaa

SEQ ID NO: 118 - DNA, 4740 bp, Homo sapiens
ttccggtttttctcaggggacgttgaaattatttttgtaacgggagtcgggagaggacggggcgtgccccgcgtgcgcgcgcgtcgtcctccccggcgctcctccacagctcgctggctcccgccgcggaaaggcgtcatgccgcccaaaaccccccgaaaaacggccgccaccgccgccgctgccgccgcggaacccccggcaccgccgccgccgccccctcctgaggaggacccagagcaggacagcggcccggaggacctgcctctcgtcaggcttgagtttgaagaaacagaagaacctgattttactgcattatgtcagaaattaaagataccagatcatgtcagagagagagcttggttaacttgggagaaagtttcatctgtggatggagtattgggaggttatattcaaaagaaaaaggaactgtggggaatctgtatctttattgcacgagttgacctagatgagatgtcgttcactttactgagctacagaaaaacatacgaaatcagtgtccataaattctttaacttactaaaagaaattgataccagtaccaaagttgataatgctatgtcaagactgttgaagaagtatgatgtattgtttgcactcttcagcaaattggaaaggacatgtgaacttatatatttgacacaacccagcagttcgatatctactgaaataaattctgcattggtgctaaaagtttcttggatcacatttttattagctaaaggggaagtattacaaatggaagatgatctggtgatttcatttcagttaatgctatgtgtccttgactattttattaaactctcacctcccatgttgctcaaagaaccatataaaacagctgttatacccattaatggttcacctcgaacacccaggcgaggtcagaacaggagtgcacggatagcaaaacaactagaaaatgatacaagaattattgaagttctctgtaaagaacatgaatgtaatatagatgaggtgaaaaatgtttatttcaaaaattttataccttttatgaattctcttggacttgtaacatctaatggacttccagaggttgaaaatctttctaaacgatacgaagaaatttatcttaaaaataaagatctagatcgaagattatttttggatcatgataaaactcttcagactgattctatagacagttttgaaacacagagaacaccacgaaaaagtaaccttgatgaagaggtgaatataattcctccacacactccagttaggactgttatgaacactatccaacaattaatgatgattttaaattctgcaagtgatcaaccttcagaaaatctgatttcctattttaacaactgcacagtgaatccaaaagaaagtatactgaaaagagtgaaggatataggatacatctttaaagagaaatttgctaaagctgtgggacagggttgtgtcgaaattggatcacagcgatacaaacttggagttcgcttgtattaccgagtaatggaatccatgcttaaatcagaagaagaacgattatccattcaaaattttagcaaacttctgaatgacaacatttttcatatgtctttattggcgtgcgctcttgaggttgtaatggccacatatagcagaagtacatctcagaatcttgattctggaacagatttgtctttcccatggattctgaatgtgcttaatttaaaagcctttgatttttacaaagtgatcgaaagttttatcaaagcagaaggcaacttgacaagagaaatgataaaacatttagaacgatgtgaacatcgaatcatggaatcccttgcatggctctcagattcacctttatttgatcttattaaacaatcaaaggaccgagaaggaccaactgatcaccttgaatctgcttgtcctcttaatcttcctctccagaataatcacactgcagcagatatgtatctttctcctgtaagatctccaaagaaaaaaggttcaactacgcgtgtaaattctactgcaaatgcagagacacaagcaacctcagccttccagacccagaagccattgaaatctacctctctttcactgttttataaaaaagtgtatcggctagcctatctccggctaaatacactttgtgaacgccttctgtctgagcacccagaattagaacatatcatctggacccttttccagcacaccctgcagaatgagtatgaactcatgagagacaggcatttggaccaaattatgatgtgttccatgtatggcatatgcaaagtgaagaatatagaccttaaattcaaaatcattgtaacagcatacaaggatcttcctcatgctgttcaggagacattcaaacgtgttttgatcaaagaagaggagtatgattctattatagtattctataactcggtcttcatgcagagactgaaaacaaatattttgcagtatgcttccaccaggccccctaccttgtcaccaatacctcacattcctcgaagcccttacaagtttcctagttcacccttacggattcctggagggaacatctatatttcacccctgaagagtccatataaaatttcagaaggtctgccaacaccaacaaaaatgactccaagatcaagaatcttagtatcaattggtgaatcattcgggacttctgagaagttccagaaaataaatcagatggtatgtaacagcgaccgtgtgctcaaaagaagtgctgaaggaagcaaccctcctaaaccactgaaaaaactacgctttgatattgaaggatcagatgaagcagatggaagtaaacatctcccaggagagtccaaatttcagcagaaactggcagaaatgacttctactcgaacacgaatgcaaaagcagaaaatgaatgatagcatggatacctcaaacaaggaagagaaatgaggatctcaggaccttggtggacactgtgtacacctctggattcattgtctctcacagatgtgactgtataactttcccaggttctgtttatggccacatttaatatcttcagctctttttgtggatataaaatgtgcagatgcaattgtttgggtgagtcctaagccacttgaaatgttagtcattgttatttatacaagattgaaaatcttgtgtaaatcctgccatttaaaaagttgtagcagattgtttcctcttccaaagtaaaattgctgtgctttatggatagtaagaatggccctagagtgggagtcctgataacccaggcctgtctgactactttgccttcttttgtagcatataggtgatgtttgctcttgtttttattaatttatatgtatatttttttaatttaacatgaacacccttagaaaatgtgtcctatctatcttccaaatgcaatttgattgactgcccattcaccaaaattatcctgaactcttctgcaaaaatggatattattagaaattagaaaaaaattactaattttacacattagattttattttactattggaatctgatatactgtgtgcttgttttataaaattttgcttttaattaaataaaagctggaagcaaagtataaccatatgatactatcatactactgaaacagatttcatacctcagaatgtaaaagaacttactgattattttcttcatccaacttatgtttttaaatgaggattattgatagtactcttggtttttataccattcagatcactgaatttataaagtacccatctagtacttgaaaaagtaaagtgttctgccagatcttaggtatagaggaccctaacacagtatatcccaagtgcactttctaatgtttctgggtcctgaagaattaagatacaaattaattttactccataaacagactgttaattataggagccttaatttttttttcatagagatttgtctaattgcatctcaaaattattctgccctccttaatttgggaaggtttgtgttttctctggaatggtacatgtcttccatgtatcttttgaactggcaattgtctatttatcttttatttttttaagtcagtatggtctaacactggcatgttcaaagccacattatttctagtccaaaattacaagtaatcaagggtcattatgggttaggcattaatgtttctatctgattttgtgcaaaagcttcaaattaaaacagctgcattagaaaaagaggcgcttctcccctcccctacacctaaaggtgtatttaaactatcttgtgtgattaacttatttagagatgctgtaacttaaaataggggatatttaaggtagcttcagctagcttttaggaaaatcactttgtctaactcagaattatttttaaaaagaaatctggtcttgttagaaaacaaaattttattttgtgctcatttaagtttcaaacttactattttgacagttattttgataacaatgacactagaaaacttgactccatttcatcattgtttctgcatgaatatcatacaaatcagttagtttttaggtcaagggcttactatttctgggtcttttgctactaagttcacattagaattagtgccagaattttaggaacttcagagatcgtgtattgagatttcttaaataatgcttcagatattattgctttattgcttttttgtattggttaaaactgtacatttaaaattgctatgttactattttctacaattaatagtttgtctattttaaaataaattagttgttaagagtc

SEQ ID NO: 119 - DNA, 2772 bp, Homo sapiens
ggtgtccggagccgcggacctagagatcccagaagccacagcgcagcggcccggcccgccactatttccaggctctgcggggcaggggccgccgcccaccgcccgccgccatggggctgaaggccgcccagaagacgctgttcccgctgcgctccatcgacgacgtggtgcgcctgtttgctgccgagctgggccgagaggagccggacctggtgctcctttccttggtgctgggcttcgtggagcattttctggctgtcaaccgcgtcatccctaccaacgttcccgagctcaccttccagcccagccccgcccccgacccgcctggcggcctcacctactttcccgtggccgacctgtctatcatcgccgccctctatgcccgcttcaccgcccagatccgaggcgccgtcgacctgtccctctatcctcgagaagggggtgtctccagccgtgagctggtgaagaaggtctccgatgtcatatggaacagcctcagccgctcctacttcaaggatcgggcccacatccagtccctcttcagcttcatcacaggcaccaaattggacagctccggtgtggcctttgctgtggttggggcctgccaggccctgggtctccgggatgtccacctcgccctgtctgaggatcatgcctgggtagtgtttgggcccaatggggagcagacagctgaggtcacctggcacggcaagggcaacgaggaccgcaggggccagacagtcaatgccggtgtggctgagcggagctggctgtacctgaaaggatcatacatgcgctgtgaccgcaagatggaggtggcgttcatggtgtgtgccatcaacccttccattgacctgcacaccgactcgctggagcttctgcagctgcagcagaagctgctctggctgctctatgacctgggacatctggaaaggtaccccatggccttagggaacctggcagatctagaggagctggagcccacccctggccggccagacccactcaccctctaccacaagggcattgcctcagccaagacctactatcgggatgaacacatctacccctacatgtacctggctggctaccactgtcgcaaccgcaatgtgcgggaagccctgcaggcctgggcggacacggccactgtcatccaggactacaactactgccgggaagacgaggagatctacaaggagttctttgaagtagccaatgatgtcatccccaacctgctgaaggaggcagccagcttgctggaggcgggcgaggagcggccgggggagcaaagccagggcacccagagccaaggttccgccctccaggaccctgagtgcttcgcccacctgctgcgattctacgacggcatctgcaaatgggaggagggcagtcccacgcctgtgctgcacgtgggctgggccacctttcttgtgcagtccctaggccgttttgagggacaggtgcggcagaaggtgcgcatagtgagccgagaggccgaggcggccgaggccgaggagccgtggggcgaggaagcccgggaaggccggcggcggggcccacggcgggagtccaagccagaggagcccccgccgcccaagaagccagcactggacaagggcctgggcaccggccagggtgcagtgtcaggacccccccggaagcctcctgggactgtcgctggcacagcccgaggccctgaaggtggcagcacggctcaggtgccagcacccgcagcatcaccaccgccggagggtccagtgctcactttccagagtgagaagatgaagggcatgaaggagctgctggtggccaccaagatcaactcgagcgccatcaagctgcaactcacggcacagtcgcaagtgcagatgaagaagcagaaagtgtccacccctagtgactacactctgtctttcctcaagcggcagcgcaaaggcctctgaactactggggacttcggaccgcttgtggggacccaggctccgccttagtcccccaactctgagcccatgttctgcccccagcccaaaggggacaggcctcacctctacccaaaccctaggttcccggtcccgagtacagtctgtatcaaacccacgattttctccagctcagaacccagggctctgccccagtcgttagaatataggtctcttctcccagaatcccagccggccaatggaaacctcacgctgggtcctaattaccagtctttaaaggcccagcccctagaaacccaagctcctcctcggaaccgctcacctagagccagaccaacgttactcagggctcctcccagcttgtaggagctgaggtttcacccttaacccaagggagcacaggtcccacctccagcccggggagcctaggaccactcagcccctaggagtatatttccgcacttcagaattccatatcttgcgaatccaagctccctgccccaaataacttcagtcctgcttccagaatttggaaatcctagtttcctctccttcgtatcccgagtctgggacacaaaactccgcccccagcctatgagcatcctgagccccgccctcttcctgacgaaactggccccggatcagagcaggacctcccttccgaccctctgggaacctcccagaggtccagcccatctcggagcatcccggaggaaatctgcagaggggttaggagtgggtgacaagagcctgatctcttcctgttttgtacatagatttatttttcagttccaagaaagatgaatacattttgttaaaaaaaaaaaaaaaa

SEQ ID NO: 120 - DNA, 567 bp, Homo sapiens
cagcgggcacgtgacatggccccggggagccgaggtgagcgtccagcttccggagccggagggggcccggcgtacccagcccccagcccgacgtgaccatgctgtcccgcctcctaaaagaacaccaggccaagcagaatgaacgcaaggagctgcaggaaaagaggaggcgagaggctatcactgcagcgacctgcctgacagaagctttggtggatcacctcaatgtgggtgtggcccaggcctacatgaaccagagaaagctggaccatgaggtgaagaccctacaggtccaggctgcccaatttgccaagcagacaggccagtggatcggaatggtggagaacttcaaccaggcactcaaggaaattggggatgtggagaactgggctcggagcatcgagctggacatgcgcaccattgccactgcactggaatatgtctacaaagggcagctgcagtctgccccttcctagcccctgttccctccccaaaccctatccctcctacctcacccgcagggggaaggagggaggctgacaagccttgaataaaacacaagcctcc

SEQ ID NO: 121 - DNA, 3074 bp, Homo sapiens
ccgctctccgctgcgggggaggccatggcggaaccttcccaggccccgaccccggccccggctgcgcagccccggccccttcagtccccagcccctgccccaactccgactcctgcacccagcccggcttcagccccgattccgactcccaccccggcaccagcccctgccccagctgcagccccagccggcagcacagggactggggggcccggggtaggaagtgggggggccgggagcgggggggatccggctcggcctggcctgagccagcagcagcgcgccagtcagaggaaggcgcaagtccgggggctgccccgcgccaagaagcttgagaagctaggggtcttctcggcttgcaaggccaatggaacctgtaagtgtaatggctggaaaaaccccaagccccccactgcaccccgcatagatctgcagcagccagctgccaacctgagtgagctgtgccgcagttgtgagcaccccttggctgaccacgtatcccacttggagaatgtgtcagaggatgagataaaccgactgctggggatggtggtggatgtggagaatctcttcatgtctgttcacaaggaagaggacacagacaccaagcaggtctatttctacctcttcaagctactgcggaaatgcatcctgcagatgacccggcctgtggtggaggggtccctgggcagccctccatttgagaaacctaatattgagcagggtgtgctgaactttgtgcagtacaagtttagtcacctggctccccgggagcggcagacgatgttcgagctctcaaagatgttcttgctctgccttaactactgggagcttgagacacctgcccagtttcggcagaggtctcaggctgaggacgtggctacctacaaggtcaattacaccagatggctctgttactgccacgtgccccagagctgtgatagcctcccccgctacgaaaccactcatgtctttgggcgaagccttctccggtccattttcaccgttacccgccggcagctgctggaaaagttccgagtggagaaggacaaattggtgcccgagaagaggaccctcatcctcactcacttccccaaattcctgtccatgctggaggaggagatctatggggcaaactctccaatctgggagtcaggcttcaccatgccaccctcagaggggacacagctggttccccggccagcttcagtcagtgcagcggttgttcccagcacccccatcttcagccccagcatgggtgggggcagcaacagctccctgagtctggattctgcaggggccgagcctatgccaggcgagaagaggacgctcccagagaacctgaccctggaggatgccaagcggctccgtgtgatgggtgacatccccatggagctggtcaatgaggtcatgctgaccatcactgaccctgctgccatgctggggcctgagacgagcctgctttcggccaatgcggcccgggatgagacagcccgcctggaggagcgccgcggcatcatcgagttccatgtcatcggcaactcactgacgcccaaggccaaccggcgggtgttgctgtggctcgtggggctgcagaatgtcttttcccaccagctgccgcgcatgcctaaggagtatatcgcccgcctcgtctttgacccgaagcacaagactctggccttgatcaaggatgggcgggtcatcggtggcatctgcttccgcatgtttcccacccagggcttcacggagattgtcttctgtgctgtcacctcgaatgagcaggtcaagggttatgggacccacctgatgaaccacctgaaggagtatcacatcaagcacaacattctctacttcctcacctacgccgacgagtacgccatcggctacttcaaaaagcagggtttctccaaggacatcaaggtgcccaagagccgctacctgggctacatcaaggactacgagggagcgacgctgatggagtgtgagctgaatccccgcatcccctacacggagctgtcccacatcatcaagaagcagaaagagatcatcaagaagctgattgagcgcaaacaggcccagatccgcaaggtctacccggggctcagctgcttcaaggagggcgtgaggcagatccctgtggagagcgttcctggcattcgagagacaggctggaagccattggggaaggagaaggggaaggagctgaaggaccccgaccagctctacacaaccctcaaaaacctgctggcccaaatcaagtctcaccccagtgcctggcccttcatggagcctgtgaagaagtcggaggcccctgactactacgaggtcatccgcttccccattgacctgaagaccatgactgagcggctgcgaagccgctactacgtgacccggaagctctttgtggccgacctgcagcgggtcatcgccaactgtcgcgagtacaaccccccggacagcgagtactgccgctgtgccagcgccctggagaagttcttctacttcaagctcaaggagggaggcctcattgacaagtaggcccatctttgggccgcagccctgacctggaatgtctccacctcggattctgatctgatccttagggggtgccctggccccacggacccgactcagcttgagacactccagccaagggtcctccggacccgatcctgcagctctttctggaccttcaggcacccccaagcgtgcagctctgtcccagccttcactgtgtgtgagaggtctcctgggttggggcccagcccctctagagtagctggtggccagggatgaaccttgcccagccgtggtggcccccaggcctggtccccaagagctttggaggcttggattcctgggcctggcccaggtggctgtttccctgaggaccagaactgctcattttagcttgagtgatggcttcaggggttggaagttcagcccaaactgaagggggccatgccttgtccagcactgttctgtcagtctcccccaggggtggggggtatggggaccattcattccctggcattaatcccttagagggaataataaagctttttatttctctg

SEQ ID NO: 122 - DNA, 3014 bp, Homo sapiens
ggggccgcgtcgacgcggaaaagaggccgtggggggcctcccagcgctggcagacaccgtgaggctggcagccgccggcacgcacacctagtccgcagtcccgaggaacatgtccgcagccagggcgcggagcagagtcccgggcaggagaaccaagggagggcgtgtgctgtggcggcggcggcagcggcagcggagccgctagtcccctccctcctgggggagcagctgccgccgctgccgccgccgccaccaccatcagcgcgcggggcccggccagagcgagccgggcgagcggcgcgctagggggagggcgggggcggggaggggggtgggcgaagggggcgggagggcgtggggggagggtctcgctctcccgactaccagagcccgagggagaccctggcggcggcggcggcgcctgacactcggcgcctcctgccgtgctccggggcggcatgtccgaggctggcggggccgggccgggcggctgcggggcaggagccggggcaggggccgggcccggggcgctgcccccgcagcctgcggcgcttccgcccgcgcccccgcagggctccccctgcgccgctgccgccgggggctcgggcgcctgcggtccggcgacggcagtggctgcagcgggcacggccgaaggaccgggaggcggtggctcggcccgaatcgccgtgaagaaagcgcaactacgctccgctccgcgggccaagaaactggagaaactcggagtgtactccgcctgcaaggccgaggagtcttgtaaatgtaatggctggaaaaaccctaacccctcacccactccccccagagccgacctgcagcaaataattgtcagtctaacagaatcctgtcggagttgtagccatgccctagctgctcatgtttcccacctggagaatgtgtcagaggaagaaatgaacagactcctgggaatagtattggatgtggaatatctctttacctgtgtccacaaggaagaagatgcagataccaaacaagtttatttctatctatttaagctcttgagaaagtctattttacaaagaggaaaacctgtggttgaaggctctttggaaaagaaacccccatttgaaaaacctagcattgaacagggtgtgaataactttgtgcagtacaaatttagtcacctgccagcaaaagaaaggcaaacaatagttgagttggcaaaaatgttcctaaaccgcatcaactattggcatctggaggcaccatctcaacgaagactgcgatctcccaatgatgatatttctggatacaaagagaactacacaaggtggctgtgttactgcaacgtgccacagttctgcgacagtctacctcggtacgaaaccacacaggtgtttgggagaacattgcttcgctcggtcttcactgttatgaggcgacaactcctggaacaagcaagacaggaaaaagataaactgcctcttgaaaaacgaactctaatcctcactcatttcccaaaatttctgtccatgctagaagaagaagtatatagtcaaaactctcccatctgggatcaggattttctctcagcctcttccagaaccagccagctaggcatccaaacagttatcaatccacctcctgtggctgggacaatttcatacaattcaacctcatcttcccttgagcagccaaacgcagggagcagcagtcctgcctgcaaagcctcttctggacttgaggcaaacccaggagaaaagaggaaaatgactgattctcatgttctggaggaggccaagaaaccccgagttatgggggatattccgatggaattaatcaacgaggttatgtctaccatcacggaccctgcagcaatgcttggaccagagaccaattttctgtcagcacactcggccagggatgaggcggcaaggttggaagagcgcaggggtgtaattgaatttcacgtggttggcaattccctcaaccagaaaccaaacaagaagatcctgatgtggctggttggcctacagaacgttttctcccaccagctgccccgaatgccaaaagaatacatcacacggctcgtctttgacccgaaacacaaaacccttgctttaattaaagatggccgtgttattggtggtatctgtttccgtatgttcccatctcaaggattcacagagattgtcttctgtgctgtaacctcaaatgagcaagtcaagggctatggaacacacctgatgaatcatttgaaagaatatcacataaagcatgacatcctgaacttcctcacatatgcagatgaatatgcaattggatactttaagaaacagggtttctccaaagaaattaaaatacctaaaaccaaatatgttggctatatcaaggattatgaaggagccactttaatgggatgtgagctaaatccacggatcccgtacacagaattttctgtcatcattaaaaagcagaaggagataattaaaaaactgattgaaagaaaacaggcacaaattcgaaaagtttaccctggactttcatgttttaaagatggagttcgacagattcctatagaaagcattcctggaattagagagacaggctggaaaccgagtggaaaagagaaaagtaaagagcccagagaccctgaccagctttacagcacgctcaagagcatcctccagcaggtgaagagccatcaaagcgcttggcccttcatggaacctgtgaagagaacagaagctccaggatattatgaagttataaggttccccatggatctgaaaaccatgagtgaacgcctcaagaataggtactacgtgtctaagaaattattcatggcagacttacagcgagtctttaccaattgcaaagagtacaacgccgctgagagtgaatactacaaatgtgccaatatcctggagaaattcttcttcagtaaaattaaggaagctggattaattgacaagtgattttttttccccctctgcttcttagaaactcaccaagcagtgtgcctaaagcaaggt

SEQ ID NO: 123 - DNA, 7869 bp, Homo sapiens
ggcacgaggtttggggcatctccgcggtccggcccggggccccgggatctcggctgtccttcctcccggcataagatgcacatttttctgctctggagccgggaatgaaatattcttgagttcttacaactttatgacgagacccatgtgtggtgctattgagaaattcattgggaagttggaagacatttcaaacaacaggttgttttggtttctatagtacaattggggtggcattctgttttgtgaaaggaggaaggacttaggccagaaaactcatatgctatggttaactggttcccagcctccgagaatcttgttttccatggtgtaaaacttactcagcatcaggataagggataacgactctatggatatacagaatccttcaccatggtaaaactcgcaaacccgctttatactgagtggattttggaggccatcaaaaaagtgaaaaagcagaaacagcgtccttcagaagaaaggatatgcaatgctgtgtcttcatcccatggcttggatcgtaaaactgttttagaacaattggagttgagtgttaaagatggaacaattttaaaagtctcaaataaaggactcaattcctataaagatcctgataatcctgggcgaatagcacttcctaagcctcggaaccatggaaaattggataataaacaaaatgtggattggaataaactgataaagcgggcagttgagggcttggcagagtctggtggctcaactttgaaaagcattgaacgttttttgaaaggtcagaaggatgtgtctgcattattcggaggcagtgctgcctctggctttcaccagcagttacgattggctatcaaacgtgccattggccacggcagactccttaaagatggacctctttatcggctcaacactaaagcaaccaacgtggatgggaaagagagttgtgagtctctttcctgtttacctccagtgtcccttcttccacatgaaaaggataagccggttgctgaaccaatccccatctgtagtttctgtcttggtacaaaagaacaaaaccgagaaaagaagccagaggaactcatctcctgtgccgactgtggcaacagtggccatccatcctgtttaaagttttcccctgaactaacggttcgagtgaaggccttacggtggcagtgcatcgagtgtaaaacatgcagctcctgtcgagatcaaggcaaaaatgcggataacatgctcttttgtgattcatgtgaccgaggttttcacatggagtgttgtgatccgccactcacccgtatgccaaaaggcatgtggatatgtcaaatatgtcgacctaggaaaaaaggacgaaaacttctacaaaagaaggcagcacagataaaacggcgctatactaatccaataggacgtccaaaaaacaggttaaagaaacaaaacacggtatcaaaaggtcccttcagcaaagttcgaactggccctggaaggggtaggaaacgaaaaatcactctttccagccaatcagcatcatcatcatcagaagaaggatatttagagcggatagatggcttggacttctgcagagatagcaatgtctccttgaggttcaacaagaaaaccaaagggctcattgatggccttaccaaattttttaccccttcccctgatgggcggaaagctcggggggaagtggtggactactctgagcaatatcgaatcagaaagaggggcaacaggaaatcaagcacttcagattggcccacagacaatcaggatggctgggatggcaaacaagaaaatgaggagcgactttttgggagccaggaaatcatgactgagaaagatatggaattatttcgtgatatccaagaacaagcactgcagaaagttggagtgactggtccccctgatccacaagtccgctgtccctctgtcattgagtttgggaagtatgaaattcacacctggtactcctccccatatcctcaagaatactcaaggctgcccaaattgtatctttgtgaattttgtctaaaatatatgaaaagtagaactattctgcagcagcacatgaagaaatgtggttggttccatcctcctgccaatgagatttacagaaagaataatatttctgtctttgaggttgatgggaatgtgagtaccatttattgtcaaaacctgtgtcttttggcaaagttgtttcttgaccacaaaaccctctattacgatgtggagccatttcttttttatgtactaacacagaatgatgtcaagggctgccaccttgttggctacttttctaaggaaaagcactgccaacagaagtacaatgtttcctgtataatgattcttcctcaataccagcgtaagggctatggcaggtttctcatcgatttcagttatttgttatcaaagcgtgaaggccaagcagggtctccagagaaaccgttatctgatctgggtcgtctttcctacatggcatattggaaaagtgtaatattggagtgcctttatcaccaaaatgacaagcagatcagcattaagaagttaagcaagttgactggaatctgccctcaagacatcacttccacactccaccacctacgaatgctggacttccgtagtgaccaatttgtgattatccgccgggaaaaacttatccaggatcacatggcaaagcttcagctgaatttgcgacctgtagatgtagatccagaatgtttgcgctggactccagtcatagtgtccaactctgtggtctcagaggaggaagaagaggaggctgaggaaggagaaaacgaagagccacagtgccaggaaagagaattagagatcagtgtgggaaagtctgtgtctcatgagaacaaagaacaagattcttattcagtagaaagtgaaaagaaaccagaagttatggctccagtcagttctacacgtttgagcaaacaagtccttcctcatgatagtcttcctgcaaatagccagccatctcggaggggccgctgggggaggaagaacagaaaaacccaggaacgttttggtgataaagattctaaactgctcttggaagagacgtcttcagctcctcaggaacaatatggagaatgtggggagaaatcagaagccacccaggaacaatacactgaaagtgaagaacagctggtggcttctgaggagcagccaagccaggacgggaaacctgaccttcccaagagaagactcagtgagggggttgagccctggcgaggacagctcaagaaaagccctgaggctctgaagtgcagattaacagaaggaagtgagaggctgccccgtcgctacagtgagggtgacagggctgtcctcaggggcttcagtgagagcagcgaggaggaggaggagccggaaagccctcggtcaagctcgccaccaattctcacaaagcccacgctgaagcgaaagaaaccatttctccaccgaaggaggagagtccgaaagcgcaaacaccacaatagcagtgtagtcacagaaactatttctgagaccactgaagtgttagatgaaccttttgaagattctgactccgagaggccaatgccaagattagaacccacatttgagatcgatgaagaagaggaggaagaggatgaaaatgaacttttccctagagaatacttccgtcgtttgtcttcgcaggatgtactcaggtgtcagtcctcttctaagaggaagtctaaagatgaagaagaagatgaagagtcagatgatgctgatgacactcctatcttaaagccagtatctcttttgcgaaaacgtgatgtgaagaattctcctcttgagccagatacatccacacctttgaaaaagaaaaagggatggcccaaaggcaagagccgcaaaccaatccactggaagaaaagacctggtcgaaaaccaggatttaagttgagtcgggaaatcatgccagtttctactcaagcatgcgtcattgagcccatcgtttccattcctaaagctggacgtaaacccaagatccaggagagtgaagaaactgttgagccaaaagaagacatgcccctacccgaggagaggaaggaggaggaggagatgcaagcagaggcagaagaggctgaagagggtgaggaagaggatgcagccagcagtgaagtcccagcagcctctccagcagacagcagcaatagtcctgagaccgaaaccaaggagcctgaggtggaggaggaagaagagaagccccgtgtctcagaggagcagaggcagtcagaggaggagcagcaggaattagaggagccagagccagaggaggaggaagatgcagctgcagagactgcccagaatgacgaccacgacgctgatgatgaggatgatggccacctggagtccacaaagaaaaaggagctagaggaacagcccacgagggaagatgtcaaggaggagcctggtgttcaagagtcttttttagatgctaatatgcagaagagtagggaaaagataaaggataaagaggaaaccgagctggattccgaagaggagcagccttcccatgacacgtccgtggtgtcagagcagatggctgggtctgaggacgaccacgaagaagactcccacactaaggaagagttaatcgaattaaaagaggaggaagagattcctcatagtgagctggatctggaaactgtacaggcagtgcagtctttgactcaagaagaaagcagtgagcatgagggcgcctaccaggactgtgaggaaactcttgcggcgtgtcagaccctgcagagttacacccaggctgacgaggaccctcagatgtccatggttgaagactgtcatgcgtcagaacataatagccctatctcctccgttcagtctcaccccagccagtcagtccgttcggtcagcagtcccaacgtgcctgcccttgagagtggctacacccagatcagcccagaacaaggatccctgtccgcaccctctatgcagaacatggagaccagccccatgatggatgtgccttccgtatcagaccactctcagcaggtggtggacagcggcttcagtgacctgggcagcattgagagcaccactgaaaactatgagaacccaagcagttacgactccacgatgggcggcagcatctgtgggaacagctcttcccagagcagctgctcctacggtgggctgtcgtcctccagcagcctcacccagagcagctgtgtggtcactcagcagatggccagcatgggcagcagctgcagcatgatgcagcagagcagcgtccagcctgctgccaactgcagcatcaagtcacctcagagctgcgtggtggagaggcctcccagtaaccagcagcagcagccgccaccaccgcctccacagcagccacagccgccgccgccacaaccacaaccagcaccacagcctccaccaccccagcagcagccgcaacagcagccgcagcctcagccccagcagcctccacccccaccccctccccagcagcagcccccgctgtcacagtgtagtatgaataacagtttcaccccagctcctatgatcatggagataccagaatctggaagcactgggaacataagtatctatgagaggattccaggggattttggtgccggcagctactctcaaccatcagccaccttcagcctagccaagctgcagcagctgaccaacaccattatggaccctcatgccatgccttatagccattctcctgctgtgacttcctatgcaaccagtgtttctctgtccaatacaggactggctcagctggctccatctcatcccttagctgggactcctcaagcacaagccaccatgacgccacccccaaacttggcatccactaccatgaacctcacatctcctctgcttcagtgcaacatgtctgccaccaacattggcattcctcacacgcagagattgcaagggcaaatgccagtgaaggggcacatttccatccgctccaagtctgcgccactgccctctgcggctgctcaccagcagcagctgtatggccgtagcccatcggcagttgccatgcaggctggccctcgcgcactggctgttcagcgtggcatgaacatgggggttaatctgatgcctactcccgcctataatgtcaattccatgaatatgaacaccttgaatgccatgaacagctatcgaatgacacagcccatgatgaacagcagttaccatagtaaccctgcctacatgaaccagacagcacagtatcctatgcagatgcagatgggaatgatggggagccaggcctatacccagcagcctatgcagcctaaccctcatgggaacatgatgtacacaggcccctcccatcacagctacatgaacgctgctggcgtgcccaagcagtcactcaacggaccttacatgagaagatgagcaagatgaacttgcaatcaaaaacttaaatatatataaataaaggaaccttttatactgacaaaccagagaaaaatggacctttttccagttaaaatattgctgtagatttagaggaatttttctttggtttattttattttttagaaaacctgatcttctcttttttttgggttcattttgttgtgggttttggttttcttcacaatcttgaacattttacagtagaactcatctaaaaatggatttggggatggggaaacatgcacaaaatcttttcataattaaaaagagccttactttctttacataccacatggacagaatttgtgtaaaagtgaattatctttattttaaaatgtatgtttcccctcactgtttgcagctcccaatgttgtcatttttaaatgttatatacatctcaagggttaaccagaccctttcctccaaacccaacctttcatttcctacttcattccagcaggaggcacttaggggagactcggatggggacatggagaacaacccaagctccttaaacttattattattgttaatattattattattattattaataaagtgaggcaggaaaatgcttctccttttaaaatcccctccactcctcacacacacacacctcttgaaacccttccccaagaatgtttctttatagacggacttcattgaaatctttgttgttcttgaatcaagtgtaatataatttttttcttcttttttaaaatattcccactcagcactcagagacacaaaaatactgtaagtctcaattaacagcagaatctcagagaaaagctgtttgcaatccaaatccagcctttggaggaatagagatggtcaattaacaatcaaaaagaggagattaacctcttgtttttttaccacctggtgaatcagccataacgcacacacacgccacccagcctcttgtttctagtatgtactttgaaatgctaactgagggtcttgatgcttgagcctttgactgataaaactcaaatagcagtccccagtgatttgcctcttaggttctttcttaaattgttggtggatgactgtacattttagtgatttgaaaaataactgacaaaccattgaaacagtttattttatgttggaagagatggcgcagatgtgtgtcagaagggagatcacggtgtgagtttcgtagctatttaagtgatacatacctctagtttttgtatgtcttttgagatcctgagttcatcccctgtgaatcagagtgcacaagcacctctcctgtgagtggctaatgagaagagggacagaccgaccaccagcacagtagggcagatctggacagcagaatgttataacgcaagttcatgtgttgctcccaactccattctcttttctctcgtgcaaccagtttgcccattctcttcctattacttgctccagggataggtaaaaaaaaaa

SEQ ID NO: 124 - DNA, 3504 bp, Homo sapiens
gccgctgcccgaatcggaaccgtcgggccgcagccgccggcaatgccgcgaaggaagaggaatgcaggcagtagttcagatggaaccgaagattccgatttttctacagatctcgagcacacagacagttcagaaagtgatggcacatcccgacgatctgctcgagtcacccgctcctcagccaggctaagccagagttctcaagattccagtcctgttcgaaatctgcagtcttttggcactgaggagcctgcttactctaccagaagagtgacccgtagtcagcagcagcctaccccagtgacaccgaaaaaataccctcttcggcagactcgttcatctggttcagaaactgagcaagtggttgatttttcagatagagaaactaaaaatacagctgatcatgatgagtcaccgcctcgaactccaactggaaatgcgccttcttctgagtctgacatagatatctccagccccaatgtatctcacgatgagagcattgccaaggacatgtccctgaaggactcaggcagtgatctctctcatcgccccaagcgccgtcgcttccatgaaagctacaacttcaatatgaagtgtcctacaccaggctgtaactctctaggacaccttacaggaaaacatgagagacatttctccatctcaggatgcccactgtatcataacctctcagctgacgaatgcaaggtgagagcacagagccgggataagcagatagaagaaaggatgctgtctcacaggcaagatgacaacaacaggcatgcaaccaggcaccaggcaccaacggagaggcagcttcgatataaggaaaaagtggctgaactcaggaagaaaagaaattctggactgagcaaagaacagaaagagaaatatatggaacacagacagacctatgggaacacacgggaacctcttttagaaaacctgacaagcgagtatgacttggatcttttccgaagagcacaagcccgggcttcagaggatttggagaagttaaggctgcaaggccaaatcacagagggaagcaacatgattaaaacaattgcttttggccgctatgagcttgatacctggtatcattctccatatcctgaagaatatgcacggctgggacgtctctatatgtgtgaattctgtttaaaatatatgaagagccaaacgatactccgccggcacatggccaaatgtgtgtggaaacacccacctggtgatgagatatatcgcaaaggttcaatctctgtgtttgaagtggatggcaagaaaaacaagatctactgccaaaacctgtgcctgttggccaaactttttctggaccacaagacattatattatgatgtggagcccttcctgttctatgttatgacagaggcggacaacactggctgtcacctgattggatatttttctaaggaaaagaattcattcctcaactacaacgtctcctgtatccttactatgcctcagtacatgagacagggctatggcaagatgcttattgatttcagttatttgctttccaaagtcgaagaaaaagttggctccccagaacgtccactctcagatctggggcttataagctatcgcagttactggaaagaagtacttctccgctacctgcataattttcaaggcaaagagatttctatcaaagaaatcagtcaggagacggctgtgaatcctgtggacattgtcagcactctgcaagcccttcagatgctcaaatactggaagggaaaacacctagttttaaagagacaggacctgattgatgagtggatagccaaagaggccaaaaggtccaactccaataaaaccatggatcccagctgcttaaaatggacccctcccaagggcacttaaagtgacctgtcattccgagccagcgaaccccagcagtaggaatccgtaccctagggatctgtctgtcatttctctgttgctcttgtgattggcaagtacagtatcctttgggaaggccatccccctcaggactgtcctggctccgacctttgtgtacactgcagacgctggttctgaggaactgttgtttcggcctcagtgaggttgcctggatgggatctgtattagacttgagtgcaggtctctcagcactgacccaaggagttctgttatggtactgtacctgtccagtcactggttctctcctcatgtcctctcgccccatgaggttgtgttgtgtcttctaagcgtggtactagtgcttgccacctggtcaccagacctccaaatatggctgccaccaccaggacctttccagttactccttatatgtgtgttctatggaggggcagggaaaaggtggcacttgtgagtgtgtgtggattggcagggggtccattcactttgggttccatcttgctttaaatttcttcattttgattaagagacctctttttgatctgtattgggctaaccagagccaaatacttttgaagagtttcccagggactagtcatggtaatagcatataattgatctgaatgagatggagagaagaatgaaggggtggtggttctgggtttgatttgagttcacctgtgggcagtgggcagtgggcagtgtcttggtgaaagggaacggatactactttttgcctcaccgtaaagtactcactagtaaatatttccttctctctttactcccactttttacgtttgcaggtgccaaagtaatgtccacttttccctttcatgctgcatattaactggttaattatactgcagaaaccttttcacctccactagtctgatacagtacatctgtacttccatataccttgcactgattttgtctgagtgccctgggagaagtagaaaatgattgaaagtgacttccgtatctcagcccatgactcagcaaggcagaatggccacccctgccaaagtttgcttctcttttcaacagtgcctcaccctccctctaggattaaagtgcttctgcccttccacgaactcctcctccatttcctttttgggatttgtcaccatccttctattctctggtcttctatttttggtgttgttcaagtgaaggaagagatgttccctctaatttctctctagcccattataacctgctatcttggggcaacttttgatgtatgacatgtcacccttcccaacttggtctcctccaacatgctgtcttcatgtggagccctcaccacaatccctgactccggtcatttgtgcctttctcttgtcatctctgtacactacttatattcactgtgggttgggggagctaattttaagcatgttcagtggcagctcccctccagtttcagtgtcactgttaaaatttatcaaaaagcaacttcactaggggttttcttaagggataaaggccttttacagaagctaaacccttccccacatgtggtagaatgtgctcttctatatctactcctcaataaagcatgttctctgctcaaaaaaaaaaaaaaaaaaaaaaaaaaaaaaaaaa

SEQ ID NO: 125 - DNA, 3423 bp, Homo sapiens
atatcatcgacagggagcacagtgggctttctcctcaagatgacactaattctggaatgtcaattccccgagtaaatccctcggtcaatcctagtatctctccagctcatggtgtggctcgttcatccacattgccaccatccaacagcaacatggtatccaccagaataaaccgccagcagagctcagaccttcatagcagcagtcatagtaattctagcaacagccaaggaagtttcggatgctcacccggaagtcagattgtagccaatgttgccttaaacaaaggacaggccagttcacagagcagtaaaccctctttaaacctcaataatcctcctatggaaggtacaggaatatccctagcacagttcatgtctccaaggagacaggttacttctggattggcaacaaggcccaggatgccaaacaattcctttcctcctaatatttcgacattaagctctcccgttggcatgacaagtagtgcctgtaataataataaccgatcttattcaaacatcccagtaacatctttacagggtatgaatgaaggacccaataactccgttggcttctctgccagttctccagtcctcaggcagatgagctcacagaattcacctagcagattaaatatacaaccagcaaaagctgagtccaaagataacaaagagattgcctcaactttaaatgaaatgattcaatctgacaacagctctagtgatggcaaacctctggattcagggcttctgcataacaatgacagactttcagatggagacagtaaatactctcaaaccagtcacaaactagtgcagcttttgacaacaactgccgaacagcagttacggcatgctgatatagacacaagctgcaaagatgtcctgtcttgcacaggcacttccaactctgcctctgctaactcttcaggaggttcttgtccctcttctcatagctcattgacagcacggcataaaattctacaccggctcttacaggagggtagcccctcagatatcaccactttgtctgtcgagcctgataaaaaggacagtgcatctacttctgtgtcagtgactggacaggtacaaggaaactccagtataaaactagaactggatgcttcaaagaaaaaagaatcaaaagaccatcagctcctacgctatcttttagataaagatgagaaagatttaagatcaactccaaacctgagcctggatgatgtaaaggtgaaagtggaaaagaaagaacagatggatccatgtaatacaaacccaaccccaatgacgaaggccactcctgaggaaataaaactggaggcccagagccagtttacagctgaccttgaccagtttgatcagttactgcccacgctggagaaggcagcacagttgccaggcttatgtgagacagacaggatggatggtgcggtcaccagtgtaaccatcaaatcggagatcctgccagcttcacttcagtccgccactgccagacccacttccaggctgaatagattacctgagctggaattggaagcaattgataaccaatttggacaaccaggaacaggcgatcagattccatggacaaataatacagtgacagctataaatcagagtaaatcagaagaccagtgtattagctcacaattagatgagcttctctgtccacccacaacagtagaagggagaaatgatgagaaggctcttcttgaacagctggtatccttccttagtggcaaagatgaaactgagctagctgaactagacagagctctgggaattgacaaacttgttcaggggggtggattagatgtattatcagagagatttccaccacaacaagcaacgccacctttgatcatggaagaaagacccaacctttattcccagccttactcttctccttttcctactgccaatctccctagccctttccaaggcatggtcaggcaaaaaccttcactggggacgatgcctgttcaagtaacacctccccgaggtgctttttcacctggcatgggcatgcagcccaggcaaactctaaacagacctccggctgcacctaaccagcttcgacttcaactacagcagcgattacagggacaacagcagttgatacaccaaaatcggcaagctatcttaaaccagtttgcagcaactgctcctgttggcatcaatatgagatcaggcatgcaacagcaaattacacctcagccacccctgaatgctcaaatgttggcacaacgtcagcgggaactgtacagtcaacagcaccgacagaggcagctaatacagcagcaaagagccatgcttatgaggcagcaaagctttgggaacaacctccctccctcatctggactaccagttcaaacggggaacccccgtcttcctcagggtgctccacagcaattcccctatccaccaaactatggtacaaatccaggaaccccacctgcttctaccagcccgttttcacaactagcagcaaatcctgaagcatccttggccaaccgcaacagcatggtgagcagaggcatgacaggaaacataggaggacagtttggcactggaatcaatcctcagatgcagcagaatgtcttccagtatccaggagcaggaatggttccccaaggtgaggccaactttgctccatctctaagccctgggagctccatggtgccgatgccaatccctcctcctcagagttctctgctccagcaaactccacctgcctccgggtatcagtcaccagacatgaaggcctggcagcaaggagcgataggaaacaacaatgtgttcagtcaagctgtccagaaccagcccacgcctgcacagccaggagtatacaacaacatgagcatcaccgtttccatggcaggtggaaatacgaatgttcagaacatgaacccaatgatggcccagatgcagatgagctctttgcagatgccaggaatgaacactgtgtgccctgagcagataaatgatcccgcactgagacacacaggcctctactgcaaccagctctcatccactgaccttctcaaaacagaagcagatggaacccagcaggtgcaacaggttcaggtgtttgctgacgtccagtgtacagtgaatctggtaggcggggacccttacctgaaccagcctggtccactgggaactcaaaagcccacgtcaggaccacagaccccccaggcccagcagaagagcctccttcagcagctactgactgaataaccacttttaaaggaatgtgaaatttaaataatagacatacagagatatacaaatatattatatatttttctgagatttttgatatctcaatctgcagccattcttcaggtcgtagcatttggagcaaaaaaaaaaaaaaaaaatcgatgtcgactcgacgacactacggcgacagcaacgg

SEQ ID NO: 126 - DNA, 6754 bp, Homo sapiens
gtcgacgtggcggccggcggcggctgcgggctgagcggcgagtttccgatttaaagctgagctgcgaggaaaatggcggcgggaggatcaaaatacttgctggatggtggactcagagaccaataaaaataaactgcttgaacatcctttgactggttagccagttgctgatgtatattcaagatgagtggattaggagaaaacttggatccactggccagtgattcacgaaaacgcaaattgccatgtgatactccaggacaaggtcttacctgcagtggtgaaaaacggagacgggagcaggaaagtaaatatattgaagaattggctgagctgatatctgccaatcttagtgatattgacaatttcaatgtcaaaccagataaatgtgcgattttaaaggaaacagtaagacagatacgtcaaataaaagagcaaggaaaaactatttccaatgatgatgatgttcaaaaagccgatgtatcttctacagggcagggagttattgataaagactccttaggaccgcttttacttcaggcattggatggtttcctatttgtggtgaatcgagacggaaacattgtatttgtatcagaaaatgtcacacaatacctgcaatataagcaagaggacctggttaacacaagtgtttacaatatcttacatgaagaagacagaaaggattttcttaagaatttaccaaaatctacagttaatggagtttcctggacaaatgagacccaaagacaaaaaagccatacatttaattgccgtatgttgatgaaaacaccacatgatattctggaagacataaacgccagtcctgaaatgcgccagagatatgaaacaatgcagtgctttgccctgtctcagccacgagctatgatggaggaaggggaagatttgcaatcttgtatgatctgtgtggcacgccgcattactacaggagaaagaacatttccatcaaaccctgagagctttattaccagacatgatctttcaggaaaggttgtcaatatagatacaaattcactgagatcctccatgaggcctggctttgaagatataatccgaaggtgtattcagagattttttagtctaaatgatgggcagtcatggtcccagaaacgtcactatcaagaagttaccagtgatgggatattttccccaacagcttatcttaatggccatgcagaaaccccagtatatcgattctcgttggctgatggaactatagtgactgcacagacaaaaagcaaactcttccgaaatcctgtaacaaatgatcgacatggctttgtctcaacccacttccttcagagagaacagaatggatatagaccaaacccaaatcctgttggacaagggattagaccacctatggctggatgcaacagttcggtaggcggcatgagtatgtcgccaaaccaaggcttacagatgccgagcagcagggcctatggcttggcagaccctagcaccacagggcagatgagtggagctaggtatgggggttccagtaacatagcttcattgacccctgggccaggcatgcaatcaccatcttcctaccagaacaacaactataggctcaacatgagtagccccccacatgggagtcctggtcttgccccaaaccagcagaatatcatgatttctcctcgtaatcgtgggagtccaaagatagcctcacatcagttttctcctgttgcaggtgtgcactctcccatggcatcttctggcaatactgggaaccacagcttttccagcagctctctcagtgccctgcaagccatcagtgaaggtgtggggacttcccttttatctactctgtcatcaccaggccccaaattggataactctcccaatatgaatattacccaaccaagtaaagtaagcaatcaggattccaagagtcctctgggcttttattgcgaccaaaatccagtggagagttcaatgtgtcagtcaaatagcagagatcacctcagtgacaaagaaagtaaggagagcagtgttgagggggcagagaatcaaaggggtcctttggaaagcaaaggtcataaaaaattactgcagttacttacctgttcttctgatgaccggggtcattcctccttgaccaactcccccctagattcaagttgtaaagaatcttctgttagtgtcaccagcccctctggagtctcctcctctacatctggaggagtatcctctacatccaatatgcatgggtcactgttacaagagaagcaccggattttgcacaagttgctgcagaatgggaattcaccagctgaggtagccaagattactgcagaagccactgggaaagacaccagcagtataacttcttgtggggacggaaatgttgtcaagcaggagcagctaagtcctaagaagaaggagaataatgcacttcttagatacctgctggacagggatgatcctagtgatgcactctctaaagaactacagccccaagtggaaggagtggataataaaatgagtcagtgcaccagctccaccattcctagctcaagtcaagagaaagaccctaaaattaagacagagacaagtgaagagggatctggagacttggataatctagatgctattcttggtgatctgactagttctgacttttacaataattccatatcctcaaatggtagtcatctggggactaagcaacaggtgtttcaaggaactaattctctgggtttgaaaagttcacagtctgtgcagtctattcgtcctccatataaccgagcagtgtctctggatagccctgtttctgttggctcaagtcctccagtaaaaaatatcagtgctttccccatgttaccaaagcaacccatgttgggtgggaatccaagaatgatggatagtcaggaaaattatggctcaagtatgggagactggggcttaccaaactcaaaggccggcagaatggaacctatgaattcaaactccatgggaagaccaggaggagattataatacttctttacccagacctgcactgggtggctctattcccacattgcctcttcggtctaatagcataccaggtgcgagaccagtattgcaacagcagcagcagatgcttcaaatgaggcctggtgaaatccccatgggaatgggggctaatccctatggccaagcagcagcatctaaccaactgggttcctggcccgatggcatgttgtccatggaacaagtttctcatggcactcaaaataggcctcttcttaggaattccctggatgatcttgttgggccaccttccaacctggaaggccagagtgacgaaagagcattattggaccagctgcacactcttctcagcaacacagatgccacaggcctggaagaaattgacagagctttgggcattcctgaacttgtcaatcagggacaggcattagagcccaaacaggatgctttccaaggccaagaagcagcagtaatgatggatcagaaggcaggattatatggacagacatacccagcacaggggcctccaatgcaaggaggctttcatcttcagggacaatcaccatcttttaactctatgatgaatcagatgaaccagcaaggcaattttcctctccaaggaatgcacccacgagccaacatcatgagaccccggacaaacacccccaagcaacttagaatgcagcttcagcagaggctgcagggccagcagtttttgaatcagagccgacaggcacttgaattgaaaatggaaaaccctactgctggtggtgctgcggtgatgaggcctatgatgcagccccagcagggttttcttaatgctcaaatggtcgcccaacgcagcagagagctgctaagtcatcacttccgacaacagagggtggctatgatgatgcagcagcagcaacagcagcagcagcagcagcagcagcagcaacagcaacagcaacagcaacagcagcaacagcagcaaacccaggccttcagcccacctcctaatgtgactgcttcccccagcatggatgggcttttggcaggacccacaatgccacaagctcctccgcaacagtttccatatcaaccaaattatggaatgggacaacaaccagatccagcctttggtcgagtgtctagtcctcccaatgcaatgatgtcgtcaagaatgggtccctcccagaatcccatgatgcaacacccgcaggctgcatccatctatcagtcctcagaaatgaagggctggccatcaggaaatttggccaggaacagctccttttcccagcagcagtttgcccaccaggggaatcctgcagtgtatagtatggtgcacatgaatggcagcagtggtcacatgggacagatgaacatgaaccccatgcccatgtctggcatgcctatgggtcctgatcagaaatactgctgacatctctgcaccaggacctcttaaggaaaccactgtacaaatgacactgcactaggattattgggaaggaatcattgttccaggcatccatcttggaagaaaggaccagctttgagctccatcaagggtattttaagtgatgtcatttgagcaggactggattttaagccgaagggcaatatctacgtgtttttcccccctccttctgctgtgtatcatggtgttcaaaacagaaatgttttttggcattccacctcctagggatataattctggagacatggagtgttactgatcataaaacttttgtgtcacttttttctgccttgctagccaaaatctcttaaatacacgtaggtgggccagagaacattggaagaatcaagagagattagaatatctggtttctctagttgcagtattggacaaagagcatagtcccagccttcaggtgtagtagttctgtgttgaccctttgtccagtggaattggtgattctgaattgtcctttactaatggtgttgagttgctctgtccctattatttgccctaggctttctcctaatgaaggttttcatttgccattcatgtcctgtaatacttcacctccaggaactgtcatggatgtccaaatggctttgcagaaaggaaatgagatgacagtatttaatcgcagcagtagcaaacttttcacatgctaatgtgcagctgagtgcactttatttaaaaagaatggataaatgcaatattcttgaggtcttgagggaatagtgaaacacattcctggtttttgcctacacttacgtgttagacaagaactatgatttttttttttaaagtactggtgtcaccctttgcctatatggtagagcaataatgctttttaaaaataaacttctgaaaacccaaggccaggtactgcattctgaatcagaatctcgcagtgtttctgtgaatagatttttttgtaaatatgacctttaagatattgtattatgtaaaatatgtatatacctttttttgtaggtcacaacaactcatttttacagagtttgtgaagctaaatatttaacattgttgatttcagtaagctgtgtggtgaggctaccagtggaagagacatcccttgacttttgtggcctgggggaggggtagtgcaccacagcttttccttccccaccccccagccttagatgcctcgctcttttcaatctcttaatctaaatgctttttaaagagattatttgtttagatgtaggcattttaattttttaaaaattcctctaccagaactaagcactttgttaatttggggggaaagaatagatatggggaaataaacttaaaaaaaaatcaggaatttaaaaaaaacgagcaatttgaagagaatcttttggattttaagcagtccgaaataatagcaattcatgggctgtgtgtgtgtgtgtatgtgtgtgtgtgtgtgtgtatgtttaattatgttaccttttcatcccctttaggagcgttttcagattttggttcgtaagacctgaatcccatattgagatctcgagtagaatccttggtgtggtttctggtgtctgctcagctgtcccctcattctactaatgtgatgctttcattatgtccctgtggattagaatagtgtcagttatttcttaagtaactcagtacccagaacagccagttttactgtgattcagagccacagtctaactgagcaccttttaaacccctccctcttctgccccctaccacttttctgctgttgcctctctttgacacctgttttagtcagttgggaggaagggaaaaatcaagtttaattccctttatctgggttaattcatttggttcaaatagttgacggaattgggtttctgaatgtctgtgaatttcagaggtctctgctagccttggtatcattttctagcaataactgagagccagttaattttaagaatttcacacatttagccaatctttctagatgtctctgaaggtaagatcatttaatatctttgatatgcttacgagtaagtgaatcctgattatttccagacccaccaccagagtggatcttattttcaaagcagtatagacaattatgagtttgccctctttcccctaccaagttcaaaatatatctaagaaagattgtaaatccgaaaacttccattgtagtggcctgtgcttttcagatagtatactctcctgtttggagacagaggaagaaccaggtcagtctgtctctttttcagctcaattgtatctgacccttctttaagttatgtgtgtggggagaaatagaatggtgctcttatgtcgac

SEQ ID NO: 127 - DNA, 6156 bp, Homo sapiens
ggcggccgcagcctcggctacagcttcggcggcgaaggtcagcgccgacggcagccggcacctgacggcgtgaccgacccgagccgatttctcttggatttggctacacacttatagatcttctgcactgtttacaggcacagttgctgatatgtgttcaagatgagtgggatgggagaaaatacctctgacccctccagggcagagacaagaaagcgcaaggaatgtcctgaccaacttggacccagccccaaaaggaacactgaaaaacgtaatcgtgaacaggaaaataaatatatagaagaacttgcagagttgatttttgcaaattttaatgatatagacaactttaacttcaaacctgacaaatgtgcaatcttaaaagaaactgtgaagcaaattcgtcagatcaaagaacaagagaaagcagcagctgccaacatagatgaagtgcagaagtcagatgtatcctctacagggcagggtgtcatcgacaaggatgcgctggggcctatgatgcttgaggcccttgatgggttcttctttgtagtgaacctggaaggcaacgttgtgtttgtgtcagagaatgtgacacagtatctaaggtataaccaagaagagctgatgaacaaaagtgtatatagcatcttgcatgttggggaccacacggaatttgtcaaaaacctgctgccaaagtctatagtaaatgggggatcttggtctggcgaacctccgaggcggaacagccataccttcaattgtcggatgctggtaaaacctttacctgattcagaagaggagggtcatgataaccaggaagctcatcagaaatatgaaactatgcagtgcttcgctgtctctcaaccaaagtccatcaaagaagaaggagaagatttgcagtcctgcttgatttgcgtggcaagaagagttcccatgaaggaaagaccagttcttccctcatcagaaagttttactactcgccaggatctccaaggcaagatcacgtctctggataccagcaccatgagagcagccatgaaaccaggctgggaggacctggtaagaaggtgtattcagaagttccatgcgcagcatgaaggagaatctgtgtcctatgctaagaggcatcatcatgaagtactgagacaaggattggcattcagtcaaatctatcgtttttccttgtctgatggcactcttgttgctgcacaaacgaagagcaaactcatccgttctcagactactaatgaacctcaacttgtaatatctttacatatgcttcacagagagcagaatgtgtgtgtgatgaatccggatctgactggacaaacgatggggaagccactgaatccaattagctctaacagccctgcccatcaggccctgtgcagtgggaacccaggtcaggacatgaccctcagtagcaatataaattttcccataaatggcccaaaggaacaaatgggcatgcccatgggcaggtttggtggttctgggggaatgaaccatgtgtcaggcatgcaagcaaccactcctcagggtagtaactatgcactcaaaatgaacagcccctcacaaagcagccctggcatgaatccaggacagcccacctccatgctttcaccaaggcatcgcatgagccctggagtggctggcagccctcgaatcccacccagtcagttttcccctgcaggaagcttgcattcccctgtgggagtttgcagcagcacaggaaatagccatagttataccaacagctccctcaatgcacttcaggccctcagcgaggggcacggggtctcattagggtcatcgttggcttcaccagacctaaaaatgggcaatttgcaaaactccccagttaatatgaatcctcccccactcagcaagatgggaagcttggactcaaaagactgttttggactatatggggagccctctgaaggtacaactggacaagcagagagcagctgccatcctggagagcaaaaggaaacaaatgaccccaacctgcccccggccgtgagcagtgagagagctgacgggcagagcagactgcatgacagcaaagggcagaccaaactcctgcagctgctgaccaccaaatctgatcagatggagccctcgcccttagccagctctttgtcggatacaaacaaagactccacaggtagcttgcctggttctgggtctacacatggaacctcgctcaaggagaagcataaaattttgcacagactcttgcaggacagcagttcccctgtggacttggccaagttaacagcagaagccacaggcaaagacctgagccaggagtccagcagcacagctcctggatcagaagtgactattaaacaagagccggtgagccccaagaagaaagagaatgcactacttcgctatttgctagataaagatgatactaaagatattggtttaccagaaataacccccaaacttgagagactggacagtaagacagatcctgccagtaacacaaaattaatagcaatgaaaactgagaaggaggagatgagctttgagcctggtgaccagcctggcagtgagctggacaacttggaggagattttggatgatttgcagaatagtcaattaccacagcttttcccagacacgaggccaggcgcccctgctggatcagttgacaagcaagccatcatcaatgacctcatgcaactcacagctgaaaacagccctgtcacacctgttggagcccagaaaacagcactgcgaatttcacagagcacttttaataacccacgaccagggcaactgggcaggttattgccaaaccagaatttaccacttgacatcacattgcaaagcccaactggtgctggacctttcccaccaatcagaaacagtagtccctactcagtgatacctcagccaggaatgatgggtaatcaagggatgataggaaaccaaggaaatttagggaacagtagcacaggaatgattggtaacagtgcttctcggcctactatgccatctggagaatgggcaccgcagagttcggctgtgagagtcacctgtgctgctaccaccagtgccatgaaccggccagtccaaggaggtatgattcggaacccagcagccagcatccccatgaggcccagcagccagcctggccaaagacagacgcttcagtctcaggtcatgaatatagggccatctgaattagagatgaacatggggggacctcagtatagccaacaacaagctcctccaaatcagactgccccatggcctgaaagcatcctgcctatagaccaggcgtcttttgccagccaaaacaggcagccatttggcagttctccagatgacttgctatgtccacatcctgcagctgagtctccgagtgatgagggagctctcctggaccagctgtatctggccttgcggaattttgatggcctggaggagattgatagagccttaggaatacccgaactggtcagccagagccaagcagtagatccagaacagttctcaagtcaggattccaacatcatgctggagcagaaggcgcccgttttcccacagcagtatgcatctcaggcacaaatggcccagggtagctattctcccatgcaagatccaaactttcacaccatgggacagcggcctagttatgccacactccgtatgcagcccagaccgggcctcaggcccacgggcctagtgcagaaccagccaaatcaactaagacttcaacttcagcatcgcctccaagcacagcagaatcgccagccacttatgaatcaaatcagcaatgtttccaatgtgaacttgactctgaggcctggagtaccaacacaggcacctattaatgcacagatgctggcccagagacagagggaaatcctgaaccagcatcttcgacagagacaaatgcatcagcaacagcaagttcagcaacgaactttgatgatgagaggacaagggttgaatatgacaccaagcatggtggctcctagtggtatgccagcaactatgagcaaccctcggattccccaggcaaatgcacagcagtttccatttcctccaaactacggaataagtcagcaacctgatccaggctttactggggctacgactccccagagcccacttatgtcaccccgaatggcacatacacagagtcccatgatgcaacagtctcaggccaacccagcctatcaggccccctccgacataaatggatgggcgcaggggaacatgggcggaaacagcatgttttcccagcagtccccaccacactttgggcagcaagcaaacaccagcatgtacagtaacaacatgaacatcaatgtgtccatggcgaccaacacaggtggcatgagcagcatgaaccagatgacaggacagatcagcatgacctcagtgacctccgtgcctacgtcagggctgtcctccatgggtcccgagcaggttaatgatcctgctctgaggggaggcaacctgttcccaaaccagctgcctggaatggatatgattaagcaggagggagacacaacacggaaatattgctgacactgctgaagccagttgcttcttcagctgaccgggctcacttgctcaaaacacttccagtctggagagctgtgtctatttgtttcaacccaactgacctgccagccggttctgctagagcagacaggcctggccctggttcccagggtggcgtccactcggctgtggcaggaggagctgcctcttctcttgacagtctgaagctcgcatccagacagtcgctcagtctgttccctgcattcaccttagtgcaacttagatctctcctccccaagtaaatgttgacaggccaatttcatacccatgtcagattgaatgtatttaaatgtatgtatttaaggagaaccatgctcttgttctgttcctgttcggttccagacactggtttcttgctttgttttccctggctaacagtctagtgccaaagattaagattttatctgggggaaagaaaagaattttttaaaaaattaaactaaagatgttttaagctaaagcctgaatttgggatggaagcaggacagacaccgtggacagcgctgtatttacagacacacccagtgcgtgaagaccaacaaagtcacagtcgtatctctagaaagctctaaagaccatgttggaaagagtctccagttactgaacagatgaaaaggagcctgtgagagggctgttaacattagcaaatattttttccttgttttttctttgttaaaaccaaactggttcacctgaatcatgaattgagaagaaataattttcatttctaaattaagtcccttttagtttgatcagacagcttgaatcagcatctcttcttccctgtcagcctgactcttcccttcccctctctcattccccatactccctattttcattccttttttaaaaaataatataagctacagaaaccaggtaagccctttatttccttaaatgttttgccagccacttaccaattgctaagtattgaatttcagaaaaaaaaaatgcatttactggcaaggagaagagcaaagttaaggcttgataccaatcgagctaaggatacctgctttggaagcatgtttattctgttccccagcaactctggcctccaaaatgggagaaacgccagtgtgtttaaattgatagcagatatcacgacagatttaacctctgccatgtgttttttattttgttttttagcagtgctgactaagccgaagttttgtaaggtacataaaatccaatttatatgtaaacaagcaataatttaagttgagaacttatgtgttttaattgtataatttttgtgaggtatacatattgtggaattgactcaaaaatgaggtacttcagtattaaattagatatcttcatagcaatgtctcctaaaggtgttttgtaaaggatatcaatgccttgattagacctaatttgtagacttaagactttttattttctaaaccttgtgattctgcttataagtcatttatctaatctatatgatatgcagccgctgtaggaaccaattcttgatttttatatgtttatattctttcttaatgaaccttagaaagactacatgttactaagcaggccacttttatggttgttttt

SEQ ID NO: 128 - DNA, 1647 bp, Homo sapiens; identical to SEQ ID NO: 68

SEQ ID NO: 129 - DNA, 3606 bp, Homo sapiens
gcggaagagcagaacgtttgggagtgtgcagctcctgggcccggctcaggcccgtcgcggaggcgcggcgcaggggagcgctcgggtgggagtctcgctcctccaccagtttattgcgacgtagcatccaggcctaggcctccccgtccatccccgccggactcgggcctctggcagcagcgggtgacgcagacggaacatcatgtcgtccgcggccgagcctccgccacccccgcctcccgagagcgcgccttccaagcccgcagcctcgatcgccagcggcgggagcaacagcagcaacaaaggcggccccgaaggcgtcgcggcgcaggcggttgcgtctgcggccagcgctggtcccgcagacgccgagatggaggaaatatttgatgatgcgtcacctggaaagcaaaaggaaatccaagaaccagatcctacctatgaagaaaaaatgcaaactgaccgggcaaatagattcgagtatttattaaagcagacagaactttttgcacatttcattcaacctgctgctcagaagactccaacttcacctttgaagatgaaaccagggcgcccacgaataaaaaaagatgagaagcagaacttactatccgttggcgattaccgacaccgtagaacagagcaagaggaggatgaagagctattaacagaaagctccaaagcaaccaatgtttgcactcgatttgaagactctccatcgtatgtaaaatggggtaaactgagagattatcaggtccgaggattaaactggctcatttctttgtatgagaatggcatcaatggtatccttgcagatgaaatgggcctaggaaagactcttcaaacaatttctcttcttgggtacatgaaacattatagaaacattcctgggcctcatatggttttggttcctaagtctacattacacaactggatgagtgaattcaagagatgggtaccaacacttagatctgtttgtttgataggagataaagaacaaagagctgcttttgtcagagacgttttattaccgggagaatgggatgtatgtgtaacatcttatgaaatgcttattaaagagaagtctgtgttcaaaaaatttaattggagatacttagtaatagatgaagctcacaggatcaaaaatgaaaaatctaagttgtcagaaatagtgagggaattcaagactacaaatagactattattaactggaacacctcttcagaacaacttgcatgagctgtggtcacttcttaactttctgttgccagatgtgtttaattcagcagatgactttgattcctggtttgatacaaacaactgccttggggatcaaaaactagttgagaggcttcatatggttttgcgtccattcctccttcgtcgaattaaggctgatgttgaaaagagtttgcctccaaagaaggaagtaaaaatctatgtgggcctcagcaaaatgcaaagggaatggtatactcggatattaatgaaggatatagatatactcaactcagcaggcaagatggacaaaatgaggttattgaacatcctaatgcagttgagaaaatgttgtaatcatccatatctctttgatggagcagaacctggtccaccttatacaacagatatgcatctagtaaccaacagtggcaaaatggtggttttagacaagctgctccctaagttaaaagaacaaggttcacgagtactaatcttcagtcaaatgacaagggtattggacattttggaagattattgcatgtggagaaattatgagtactgcaggttggatggtcagacaccccatgatgagagacaagactccatcaatgcatacaatgaaccaaacagcacaaagtttgttttcatgttaagcacgcgtgctggtggtcttggcatcaatcttgcgactgctgatgtagtaattttgtatgattctgattggaatccccaagtagatcttcaggctatggaccgagcacatagaattgggcagactaagacagtcagagtgttccgctttataactgataacactgtagaagaaagaatagtagaacgtgctgagatgaaactcagactggattcaatagtcattcaacaagggaggcttgtggatcagaatctgaacaaaattgggaaagatgaaatgcttcaaatgattagacatggagcaacacatgtgtttgcttcaaaggaaagtgagatcactgatgaagatatcgatggtattttggaaagaggtgcaaagaagactgcagagatgaatgaaaagctctccaagatgggcgaaagttcacttagaaactttacaatggatacagagtcaagtgtttataacttcgaaggagaagactatagagaaaaacaaaagattgcattcacagagtggattgaaccacctaaacgagaaagaaaagccaactatgccgttgatgcatatttcagggaagctcttcgtgttagtgaacctaaagcacccaaggctcctcgacctccaaaacaacccaatgttcaggatttccagttctttcctccacgtttatttgaattactggaaaaagaaattctgttttacagaaaaactattgggtacaaggtacctcgaaatcctgagctgcctaacgcagcacaggcacaaaaagaagaacagcttaaaattgatgaagctgaatcccttaatgatgaagagttagaggaaaaagagaagcttctaacacagggatttaccaattggaataagagagattttaaccagtttatcaaagctaatgagaagtggggtcgtgatgatattgaaaatatagcaagagaagtagaaggcaaaactccagaagaagtcattgaatattcagctgtgttttgggaaaggtgcaacgagctccaggacatagagaagattatggctcagattgaaaggggagaggcgagaattcaaagaagaataagcatcaagaaagcacttgacacaaagattggacggtacaaagcaccttttcatcagctgagaatatcatatggtactaacaaaggaaaaaactatactgaagaagaagatcgttttctgatttgtatgcttcacaaacttggatttgacaaagaaaatgtttatgatgaattgcgacagtgtattcgcaactctcctcagttcagatttgactggtttcttaagtccagaactgcaatggagctccagaggagatgtaataccttaattactttgattgaaagagaaaacatggaactagaagaaaaggagaaggcagagaaaaagaaacgaggaccaaagccttcaacacagaaacgtaaaatggatggcgcacctgatggtcgaggaagaaaaaagaagctgaaactatgaatatgtttttgtttcataatcactaactttaaaccagtagttctttaatttacgggtcttcataagatgtactgtacaatgctcaattgttatgtcatttaaagacatcaggttcatctgtttactgagctagaaacatagtatgtagtttcacttttttaaatgcaacagctgtgctgaaatttttttatcattaacacttgaagtaataaaataggcttcatttattaaaaaaaaaaaaaaaa

SEQ ID NO: 130 - DNA, 5257 bp, Homo sapiens
aaaaattttctgttaccaaattttacaacttctaataagactactataactttatgtaaactgatgaagatgtgctgattaacatattctgtgatatggtttacaacttttaatcataattgtccatgattttggaatgctgttatttatcagtaaatgtaaaatatttgaggcatttagccatacacacactagaactttttaaaactttgtcctatagtgtaattataaactgatgactattatcttcatacattgagtcttcatgcatcaatgaaatgaaaaatataggagtagatgtccacgcccacagaccctggtgcgatgccccacccagggccttcgccggggcctgggccttcccctgggccaattcttgggcctagtccaggaccaggaccatccccaggttccgtccacagcatgatggggccaagtcctggacctccaagtgtctcccatcctatgccgacgatggggtccacagacttcccacaggaaggcatgcatcaaatgcataagcccatcgatggtatacatgacaaggggattgtagaagacatccattgtggatccatgaagggcactggtatgcgaccacctcacccaggcatgggccctccccagagtccaatggatcaacacagccaaggttatatgtcaccacacccatctccattaggagccccagagcacgtctccagccctatgtctggaggaggcccaactccacctcagatgccaccaagccagccgggggccctcatcccaggtgatccgcaggccatgagccagcccaacagaggtccctcacctttcagtcctgtccagctgcatcagcttcgagctcagattttagcttataaaatgctggcccgaggccagcccctccccgaaacgctgcagcttgcagtccaggggaaaaggacgttgcctggcttgcagcaacaacagcagcagcaacagcagcagcagcagcagcagcagcagcagcagcagcagcaacagcagccgcagcagcagccgccgcaaccacagacgcagcaacaacagcagccggcccttgttaactacaacagaccatctggcccggggccggagctgagcggcccgagcaccccgcagaagctgccggtgcccgcgcccggcggccggccctcgcccgcgccccccgcagccgcgcagccgcccgcggccgcagtgcccgggccctcagtgccgcagccggccccggggcagccctcgcccgtcctccagctgcagcagaagcagagccgcatcagccccatccagaaaccgcaaggcctggaccccgtggaaattctgcaagagcgggaatacagacttcaggcccgcatagctcataggatacaagaactggaaaatctgcctggctctttgccaccagatttaagaaccaaagcaaccgtggaactaaaagcacttcggttactcaatttccagcgtcagctgagagaggaggtggtggcctgcatgcgcagggacacgaccctggagacggctctcaactccaaagcatacaaacggagcaagcgccagactctgagagaagctcgcatgaccgagaagctggagaagcagcagaagattgagcaggagaggaaacgccgtcagaaacaccaggaatacctgaacagtattttgcaacatgcaaaagattttaaggaatatcatcggtctgtggccggaaagatccagaagctctccaaagcagtggcaacttggcatgccaacactgaaagagagcagaagaaggagacagagcggattgaaaaggagagaatgcggcgactgatggctgaagatgaggagagttatagaaaactgattgatcaaaagaaagacaggcgtttagcttaccttttgcagcagaccgatgagtatgtagccaatctgaccaatctggtttgggagcacaagcaagcccaggcagccaaagagaagaagaagaggaggaggaggaagaagaaggctgaggagaatgcagagggtggggagtctgccctgggaccggatggagagcccatagatgagagcagccagatgagtgacctccctgtcaaagtgactcacacagaaaccggcaaggttctgttcggaccagaagcacccaaagcaagtcagctggacgcctggctggaaatgaatcctggttatgaagttgcccctagatctgacagtgaagagagtgattctgattatgaggaagaggatgaggaagaagagtccagtaggcaggaaaccgaagagaaaatactcctggatccaaatagcgaagaagtttctgagaaggatgctaagcagatcattgagacagctaagcaagacgtggatgatgaatacagcatgcagtacagtgccaggggctcccagtcctactacaccgtggctcatgccatctcggagagggtggagaaacagtctgccctcctaattaatgggaccctaaagcattaccagctccagggcctggaatggatggtttccctgtataataacaacttgaacggaatcttagccgatgaaatggggcttggaaagaccatacagaccattgcactcatcacttatctgatggagcacaaaagactcaatggcccctatctcatcattgttcccctttcgactctatctaactggacatatgaatttgacaaatgggctccttctgtggtgaagatttcttacaagggtactcctgccatgcgtcgctcccttgtcccccagctacggagtggcaaattcaatgtcctcttgactacttatgagtatattataaaagacaagcacattcttgcaaagattcggtggaaatacatgatagtggacgaaggccaccgaatgaagaatcaccactgcaagctgactcaggtcttgaacactcactatgtggcccccagaaggatcctcttgactgggaccccgctgcagaataagctccctgaactctgggccctcctcaacttcctcctcccaacaatttttaagagctgcagcacatttgaacaatggttcaatgctccatttgccatgactggtgaaagggtggacttaaatgaagaagaaactatattgatcatcaggcgtctacataaggtgttaagaccatttttactaaggagactgaagaaagaagttgaatcccagcttcccgaaaaagtggaatatgtgatcaagtgtgacatgtcagctctgcagaagattctgtatcgccatatgcaagccaaggggatccttctcacagatggttctgagaaagataagaaggggaaaggaggtgctaagacacttatgaacactattatgcagttgagaaaaatctgcaaccacccatatatgtttcagcacattgaggaatcctttgctgaacacctaggctattcaaatggggtcatcaatggggctgaactgtatcgggcctcagggaagtttgagctgcttgatcgtattctgccaaaattgagagcgactaatcaccgagtgctgcttttctgccagatgacatctctcatgaccatcatggaggattattttgcttttcggaacttcctttacctacgccttgatggcaccaccaagtctgaagatcgtgctgctttgctgaagaaattcaatgaacctggatcccagtatttcattttcttgctgagcacaagagctggtggcctgggcttaaatcttcaggcagctcatacagtggtcatctttgacagcgactggaatcctcatcaggatctgcaggcccaagaccgagctcaccgcatcgggcagcagaacgaggtccgggtactgaggctctgtaccgtgaacagcgtggaggaaaagatcctcgcggccgcaaaatacaagctgaacgtggatcagaaagtgatccaggcgggcatgtttgaccaaaagtcttcaagccacgagcggagggcattcctgcaggccatcttggagcatgaagaggaaaatgaggaagaagatgaagtaccggacgatgagactctgaaccaaatgattgctcgacgagaagaagaatttgacctttttatgcggatggacatggaccggcggagggaagatgcccggaacccgaaacggaagccccgtttaatggaggaggatgagctgccctcctggatcattaaggatgacgctgaagtagaaaggctcacctgtgaagaagaggaggagaaaatatttgggagggggtcccgccagcgccgtgacgtggactacagtgacgccctcacggagaagcagtggctaagggccatcgaagacggcaatttggaggaaatggaagaggaagtacggcttaagaagcgaaaaagacgaagaaatgtggataaagatcctgcaaaagaagatgtggaaaaagctaagaagagaagaggccgccctcccgctgagaaactgtcaccaaatccccccaaactgacaaagcagatgaacgctatcatcgatactgtgataaactacaaagatagttcagggcgacagctcagtgaagtcttcattcagttaccttcaaggaaagaattaccagaatactatgaattaattaggaagccagtggatttcaaaaaaataaaggaaaggattcgtaatcataagtaccggagcctaggcgacctggagaaggatgtcatgcttctctgtcacaacgctcagacgttcaacctggagggatcccagatctatgaagactccatcgtcttacagtcagtgtttaagagtgcccggcagaaaattgccaaagaggaagagagtgaggatgaaagcaatgaagaggaggaagaggaagatgaagaagagtcagagtccgaggcaaaatcagtcaaggtgaaaattaagctcaataaaaaagatgacaaaggccgggacaaagggaaaggcaagaaaaggccaaatcgaggaaaagccaaacctgtagtgagcgattttgacagcgatgaggagcaggatgaacgtgaacagtcagaaggaagtgggacggatgatgagtgatcagtatggacctttttccttggtagaactgaattccttcctcccctgtctcatttctacccagtgagttcatttgtcatataggcactgggttgtttctatatcatcatcgtctataaactagctttaggatagtgccagacaaacatatgatatcatggtgtaaaaaacacacacatacacaaatatttgtgaccaaatgggcctcaaagattcagattgaaacaaacaaaaagctttt

SEQ ID NO: 131 - DNA, 5247 bp, Homo sapiens
ttccggcgggggaggcgccgggaagtcgatggcgccggcggctcctgcaggaggccactgtctgcagctcccgtgaagatgtccactccagacccacccctgggcggaactcctcggccaggtccttccccgggccctggcccttcccctggagccatgctgggccctagcccgggtccctcgccgggctccgcccacagcatgatggggcccagcccagggccgccctcagcaggacaccccatccccacccaggggcctggagggtaccctcaggacaacatgcaccagatgcacaagcccatggagtccatgcatgagaagggcatgtcggacgacccgcgctacaaccagatgaaaggaatggggatgcggtcagggggccatgctgggatggggcccccgcccagccccatggaccagcactcccaaggttacccctcgcccctgggtggctctgagcatgcctctagtccagttccagccagtggcccgtcttcggggccccagatgtcttccgggccaggaggtgccccgctggatggtgctgacccccaggccttggggcagcagaaccggggcccaaccccatttaaccagaaccagctgcaccagctcagagctcagatcatggcctacaagatgctggccagggggcagcccctccccgaccacctgcagatggcggtgcagggcaagcggccgatgcccgggatgcagcagcagatgccaacgctacctccaccctcggtgtccgcaacaggacccggccctggccctggccctggccccggcccgggtcccggcccggcacctccaaattacagcaggcctcatggtatgggagggcccaacatgcctcccccaggaccctcgggcgtgccccccgggatgccaggccagcctcctggagggcctcccaagccctggcctgaaggacccatggcgaatgctgctgcccccacgagcacccctcagaagctgattcccccgcagccaacgggccgcccttcccccgcgccccctgccgtcccacccgccgcctcgcccgtgatgccaccgcagacccagtcccccgggcagccggcccagcccgcgcccatggtgccactgcaccagaagcagagccgcatcacccccatccagaagccgcggggcctcgaccctgtggagatcctgcaggagcgcgagtacaggctgcaggctcgcatcgcacaccgaattcaggaacttgaaaaccttcccgggtccctggccggggatttgcgaaccaaagcgaccattgagctcaaggccctcaggctgctgaacttccagaggcagctgcgccaggaggtggtggtgtgcatgcggagggacacagcgctggagacagccctcaatgctaaggcctacaagcgcagcaagcgccagtccctgcgcgaggcccgcatcactgagaagctggagaagcagcagaagatcgagcaggagcgcaagcgccggcagaagcaccaggaatacctcaatagcattctccagcatgccaaggatttcaaggaatatcacagatccgtcacaggcaaaatccagaagctgaccaaggcagtggccacgtaccatgccaacacggagcgggagcagaagaaagagaacgagcggatcgagaaggagcgcatgcggaggctcatggctgaagatgaggaggggtaccgcaagctcatcgaccagaagaaggacaagcgcctggcctacctcttgcagcagacagacgagtacgtggctaacctcacggagctggtgccgcagcacaaggctgcccaggtcgccaaggagaaaaagaagaaaaagaaaaagaagaaggcagaaaatgcagaaggacagacgcctgccattgggccggatggcgagcctctagacgagaccagccagatgagcgacctcccggtgaaggtgatccacgtggagagtgggaagatcctcacaggcacagatgcccccaaagccgggcagctggaggcctggctcgagatgaacccggggtatgaagtagctccgaggtctgatagtgaagaaagtggctcagaagaagaggaagaggaggaggaggaagagcagccgcaggcagcacagcctcccaccctgcccgtggaggagaagaagaagattccagatccagacagcgatgacgtctctgaggtggacgcgcggcacatcattgagaatgccaagcaagatgtcgatgatgaatatggcgtgtcccaggcccttgcacgtggcctgcagtcctactatgccgtggcccatgctgtcactgagagagtggacaagcagtcagcgcttatggtcaatggtgtcctcaaacagtaccagatcaaaggtttggagtggctggtgtccctgtacaacaacaacctgaacggcatcctggccgacgagatgggcctggggaagaccatccagaccatcgcgctcatcacgtacctcatggagcacaaacgcatcaatgggcccttcctcatcatcgtgcctctctcaacgctgtccaactgggcgtacgagtttgacaagtgggccccctccgtggtgaaggtgtcttacaagggatccccagcagcaagacgggcctttgtcccccagctccggagtgggaagttcaacgtcttgctgacgacgtacgagtacatcatcaaagacaagcacatcctcgccaagatccgttggaagtacatgattgtggacgaaggtcaccgcatgaagaaccaccactgcaagctgacgcaggtgctcaacacgcactatgtggcaccccgccgcctgctgctgacgggcacaccgctgcagaacaagcttcccgagctctgggcgctgctcaacttcctgctgcccaccatcttcaagagctgcagcaccttcgagcagtggtttaacgcaccctttgccatgaccggggaaaaggtggacctgaatgaggaggaaaccattctcatcatccggcgtctccacaaagtgctgcggcccttcttgctccgacgactcaagaaggaagtcgaggcccagttgcccgaaaaggtggagtacgtcatcaagtgcgacatgtctgcgctgcagcgagtgctctaccgccacatgcaggccaagggcgtgctgctgactgatggctccgagaaggacaagaagggcaaaggcggcaccaagaccctgatgaacaccatcatgcagctgcggaagatctgcaaccacccctacatgttccagcacatcgaggagtccttttccgagcacttggggttcactggcggcattgtccaagggctggacctgtaccgagcctcgggtaaatttgagcttcttgatagaattcttcccaaactccgagcaaccaaccacaaagtgctgctgttctgccaaatgacctccctcatgaccatcatggaagattactttgcgtatcgcggctttaaatacctcaggcttgatggaaccacgaaggcggaggaccggggcatgctgctgaaaaccttcaacgagcccggctctgagtacttcatcttcctgctcagcacccgggctggggggctcggcctgaacctccagtcggcagacactgtgatcatttttgacagcgactggaatcctcaccaggacctgcaagcgcaggaccgagcccaccgcatcgggcagcagaacgaggtgcgtgtgctccgcctctgcaccgtcaacagcgtggaggagaagatcctagctgcagccaagtacaagctcaacgtggaccagaaggtgatccaggccggcatgttcgaccagaagtcctccagccatgagcggcgcgccttcctgcaggccatcctggagcacgaggagcaggatgagagcagacactgcagcacgggcagcggcagtgccagcttcgcccacactgcccctccgccagcgggcgtcaaccccgacttggaggagccacctctaaaggaggaagacgaggtgcccgacgacgagaccgtcaaccagatgatcgcccggcacgaggaggagtttgatctgttcatgcgcatggacctggaccgcaggcgcgaggaggcccgcaaccccaagcggaagccgcgcctcatggaggaggacgagctcccctcgtggatcatcaaggacgacgcggaggtggagcggctgacctgtgaggaggaggaggagaagatgttcggccgtggctcccgccaccgcaaggaggtggactacagcgactcactgacggagaagcagtggctcaaggccatcgaggagggcacgctggaggagatcgaagaggaggtccggcagaagaaatcatcacggaagcgcaagcgagacagcgacgccggctcctccaccccgaccaccagcacccgcagccgcgacaaggacgacgagagcaagaagcagaagaagcgcgggcggccgcctgccgagaaactctcccctaacccacccaacctcaccaagaagatgaagaagattgtggatgccgtgatcaagtacaaggacagcagcagtggacgtcagctcagcgaggtcttcatccagctgccctcgcgaaaggagctgcccgagtactacgagctcatccgcaagcccgtggacttcaagaagataaaggagcgcattcgcaaccacaagtaccgcagcctcaacgacctagagaaggacgtcatgctcctgtgccagaacgcacagaccttcaacctggagggctccctgatctatgaagactccatcgtcttgcagtcggtcttcaccagcgtgcggcagaaaatcgagaaggaggatgacagtgaaggcgaggagagtgaggaggaggaagagggcgaggaggaaggctccgaatccgaatctcggtccgtcaaagtgaagatcaagcttggccggaaggagaaggcacaggaccggctgaagggcggccggcggcggccgagccgagggtcccgagccaagccggtcgtgagtgacgatgacagtgaggaggaacaagaggaggaccgctcaggaagtggcagcgaagaagactgagccccgacattccagtctcgaccccgagcccctcgttccagagctgagatggcataggccttagcagtaacgggtagcagcagatgtagtttcagacttggagtaaaactgtataaacaaaagaatcttccatatttatacagcagagaagctgtaggactgtttgtgactggccctgtcctggcatcagtagcatctgtaacagcattaactgtcttaaagaga

SEQ ID NO: 132 - DNA, 3418 bp, Homo sapiens
attcccggggtctgactggactcgcggcgacttacctttcagtcgtgcgctcctgatccggcgctcggaatttgtccccggcttcagggctgcggggcctggaaggaggcgtatcgaggcggctcgaaaacgatccaggggagccgaggcgctcctcttgtcatcccactcagcgccatgtcctggatgttcaagagggatccagtttggaagtacttgcagactgtccagtatggagttcatggaaattttccacgcctctcatatccaactttctttccacgttttgaattccaagatgttatccctccagatgactttctaactagtgatgaagaagtagattccgttttatttggaagtttgagaggtcatgtggttggactacgctattacacgggagtagttaataataatgaaatggttgcattacaacgagatcctaataacccttatgataagaatgcaattaaagtaaacaatgtgaatggaaatcaagttggccatttaaagaaagagcttgcaggtgctttggcctatatcatggacaacaaattggcacaaattgaaggggtagttccttttggtgcaaacaatgcttttaccatgcctctgcatatgactttttggggaaaagaagaaaatagaaaagcggtttcagatcagttgaagaaacatggatttaaattgggtcctgcaccaaaaactttaggattcaatttggaaagtggttggggctctggaagagctggaccaagctatagtatgccagtgcatgctgcagtacagatgacaactgaacagcttaaaacagaatttgacaaattgtttgaagatttaaaagaagatgataaaacccatgaaatggaaccagctgaggctattgaaacaccactgcttccacatcaaaaacaagctctagcttggatggtgtcacgggaaaatagcaaagaacttccaccattctgggaacagcgaaatgacttatactataacacaataacaaatttttctgagaaggaccgaccagaaaatgtccatggaggaattttagctgatgatatgggtttgggtaaaactcttacagccattgcagtaatccttaccaacttccatgatggcagacctcttcctattgaaagagttaaaaagaatctactgaagaaggaatataatgttaacgatgactctatgaaacttggaggaaacaataccagtgaaaaggcagatggactaagcaaagacgcatctagatgtagtgaacaacccagtatttcagatatcaaggagaagagtaagtttcgcatgtcagaattgtctacgtcccgccccaaaagaagaaaaactgctgtccagtacatagaaagcagtgattcagaggaaattgaaacaagtgaattgccgcagaaaatgaaaggcaaactgaaaaatgtacagtctgaaactaaaggcagggcgaaagcaggatcttctaaggttatagaagatgtggcatttgcatgtgcattaacttcatctgttcctacaacaaaaaagaaaatgttgaaaaagggagcttgtgcagtggaggggtcaaagaaaactgatgttgaggagagaccaagaacaacactgatcatctgtccgctttctgtgttaagcaactggattgaccagtttggacaacatataaaatcagatgtacacttgaatttttatgtttattatggtcctgatcgtattagagaaccggccttactttcaaaacaggatattgttttgactacgtataatattttaactcatgactatggaactaaaggagatagtccattacatagcataaggtggctaagagtgatcctggatgaaggacatgccatacgaaatccaaatgctcagcagacaaaagctgtacttgacttagaatcagaaagaagatgggttttgacaggtactccaatccagaattctttaaaggacttgtggtctcttctttcctttttaaaacttaaaccatttattgatagagaatggtggcatagaacaatacagcgtcctgtcacaatgggagatgaaggaggacttaggcgtttacagtccctaattaaaaatattacacttagaagaacaaagacaagcaaaattaaaggaaaacctgttttggagttaccagaacgtaaagtatttattcagcacattacactttcagatgaagagagaaagatttatcagtctgtgaaaaatgaaggcagagccactattggaaggtattttaatgaagggactgtcctggcacattatgcagatgtcctgggtcttttgcttagactgcggcaaatttgttgccatacttaccttcttacaaatgcagtgtcttccaatggcccctcaggaaatgatacacctgaagaactgagaaagaagttaataaggaagatgaagttaattctgagctcaggttcagatgaggaatgtgcaatttgcctggattctttaacagttcctgtgataacacattgtgcacatgtattttgtaaaccctgtatttgccaagtcattcagaatgagcagccacatgctaaatgccctttatgcagaaatgatatacatgaagataatttattagaatgtcctccagaagaattagcacgtgacagtgagaaaaagtctgatatggaatggacatccagttcaaagattaatgcgctaatgcacgcattgactgacttaagaaagaagaatcccaacataaaaagtttggttgtttctcagtttacaacattcctgtctttaatagaaataccacttaaagcctctggatttgtgtttactcgtttggatggttccatggcccaaaagaaaagagttgaatcaattcagtgttttcaaaacactgaagcaggatctccaactataatgcttctgtccttaaaagcaggtggagttggtttgaatctgtctgcagcttctcgagtgtttttaatggatccagcctggaatcctgctgctgaagatcagtgctttgacagatgccatagacttggtcagaagcaagaagttatcatcacaaaattcattgtaaaggactctgttgaagaaaatatgctgaaaatacaaaacaaaaagagagaacttgcagcaggagcctttggaactaaaaaaccaaatgctgacgaaatgaaacaagccaaaattaatgaaatcagaacattaattgacttataatttgtgggattttagtaagaagactactatatgtgagaggcgtgatatctggatggaagttgggctggatgatctccaaagtcgtttcaactcttaaagacatcttaatcctgaatgtaaacaattgttatgtgtttagaatcagaatttgattttgaacttgagtaattcatccttacagctatctgtagaattagtcatcttttttctt

SEQ ID NO: 133 - DNA, 3022 bp, Homo sapiens
gctgggttggaaaaagactatgttagcaagtgtcacgccatgcttttgccaactttccaattaaaggttgacattcctgcataaacatttctctgtgaaaatgtccttgcctcttacagaggagcagaggaaaaagattgaagagaatcgacaaaaggctctggcccgcagagctgagaagttattggcagaacagcatcagaggactagctcgggcacctccattgctggcaacccattccaggccaagcaaggcccatcccaaaatttcccaagggagtcttgtaagccagtgagccatggtgtcattttcaagcaacagaatctcagtagctcatctaatgctgaccaaagacctcatgattcccacagttttcaggcaaagggaatatggaaaaagccagaagaaatgcccacagcctgcccaggccacagtccacgtagtcaaatgactctcactggaatctctcctcccttggcacaaagtcctccagaggtccctaaacaacagctcttgagttatgagttaggtcaaggtcatgctcaggcttcacctgagatcaggttcacaccctttgctaacccaactcataagcctctggccaaaccaaagagttcccaagagacaccagctcattcctctggacagcctcccagggatgctaagttagaggccaagacagcaaaagcctccccttcggggcagaacatttcttacatccattctagctcagagagtgtaacgcccaggacagaaggaagactccagcagaagtcagggtcctcagtccaaaaaggagtgaactctcagaagggaaagtgcgtaaggaacggcgatcgtttccaggtgttgattgggtacaatgcggaactcattgcagtgtttaagaccctgcccagcaagaattatgatcctgacaccaagacgtggaacttcagcatgaatgactatagtgccctgatgaaagcagcccagagcctccccacggtcaacctgcagcctctggaatgggcctatgggagcagcgagtcaccctccaccagcagtgagggacaggccggccttccatcagctccatccctttcatttgtcaaagggcgaggcatgctcatctccagggcctacttcgaggcagacatcagttattcacaggaccttattgcgctttttaaacagatggattccagaagatatgatgtcaagaccaggaagtggagctttctcttggaagagcacagtaaactaattgcaaaggtgcgctgcctcccacaagttcagctggaccctctgcccacgactctcaccctggcgtttgcttctcagctcaagaagacatctctcagtctcacgccagatgtcccagaggcagacctttctgaagtggaccccaagctcgtgtctaatctgatgccctttcagagagctggagtcaattttgccatagccaaaggaggccgcctgctgctcgctgacgacatgggcctggggaagaccatccaagccatctgcatcgcagccttttaccggaaggagtggccgctcctggtggtggtgccatcctccgtgcgcttcacctgggagcaggccttccttcggtggctgccatctctgagcccagattgcatcaacgtcgtggtgactgggaaggaccgcctgacagctggcctgatcaacattgtcagctttgaccttcttagcaagttggaaaaacagctaaaaaccccttttaaagttgtcatcattgatgaatctcacttcctcaaaaacagtaggactgcccgctgtcgagcagctatgccggtcctaaaggtcgccaagagggtgatcctgttgtcgggcacaccagccatgtcccggcccgcagagctctacacgcagatcatcgcagtcaagccaactttcttcccccagtttcatgcctttggacttcgctactgtgatgccaaacggatgccttgggggtgggactactcaggttcctccaacctgggagagctgaagctcctgctggaggaagcagtcatgctgcggcgcctcaagtccgacgtcctttcccagctgcctgccaagcagcgcaagatagtggtgattgccccaggacggatcaatgccaggaccagagctgccctggatgctgcagccaaggaaatgaccaccaaggacaaaactaaacagcagcagaaagatgccctcattctcttcttcaacagaacagctgaagctaaaatcccatctgtcattgaatatatcttggacctactggaaagtggaagagagaagtttttagtatttgcacaccataaggtggtcctggacgcaattacgcaagagcttgagagaaagcacgtgcagcacatccgcatcgatggctccacctcatcagctgagcgggaggacctgtgccagcagttccaactgtcggagaggcatgctgtggccgtgctgtccatcaccgctgccaatatgggcctcaccttctcctcggctgacctggtggtgtttgctgagctgttttggaacccaggggtgctgatccaggctgaggaccgcgtgcaccgcattggacagaccagctccgtgggcattcactacctcgtggcaaagggcacagctgatgactacctttggcccctgattcaagagaagattaaagttctggcagaagccgggctttctgagaccaatttttcagaaatgacagaatccactgattacctctacaaggacccaaagcagcagaagatgtacgacctattccagaagtcctttgagaaagaaggaagtgatatggagctcgtggaagcagcagagtcctttgacccaggaagtggttcaggaacatctggaagtagttcccagaacatgggagacaccctggatgaaagctcattgacagccagtccacagaagaaaaggagatttgaattttttgataactgggacagctttacgtctcccctgtaaaaggggcaaaaaaaaaaaaaaaaaaagcattttaaaatcaaaaagaaaaaaataaaa

SEQ ID NO: 134 - DNA, 3634 bp, Homo sapiens
caagactggaagcagagagagagagcaagagtgagagagagcgagcgagcgtagtcaggagatggtgttgtattccaggagaaaaacgtttcttcatttcaactcaaacttgctgctaaagcgcctaaatctgaaaaggaaatggacccagaatatgaagagaaaatgaaagccgaccgagcaaagagatttgaatttttactgaagcagacagaactttttgcacatttcattcagccttcagcacagaaatctccaacatctccactgaacatgaaattgggacgtccccgaataaagaaagatgaaaagcagagcttaatttctgctggagactaccgccataggcgcacagagcaagaagaagatgaagagctactgtctgagagtcggaaaacatctaatgtgtgtattagatttgaggtgtcaccttcatatgtgaaaggggggccactgagagattatcagattcgaggactgaattggttgatctctttatatgaaaatggagtcaatggcattttggctgatgaaatgggccttgggaaaactttacaaacaattgctttgcttggttacctgaaacactaccgaaatattcctggacctcacatggttttagttccaaagtctactttacacaactggatgaatgaatttaaacgatgggtcccatctctccgtgtcatttgttttgtcggagacaaggatgccagagctgcttttattcgtgatgaaatgatgccaggagagtgggatgtttgcgttacttcttatgagatggtaattaaagaaaaatctgtattcaaaaagtttcactggcgatacctggtcattgatgaagctcacagaataaagaatgaaaaatctaagctttcagagattgttcgtgagttcaagtcgactaaccgcttgctcctaactggaacacctttgcagaataacctgcatgaactgtgggccttactcaactttttattgcctgatgtctttaattctgcagatgactttgattcttggtttgacactaaaaattgtcttggtgatcaaaaactcgtggaaagacttcatgcagttttaaaaccatttttgttacgccgtataaaaactgatgtagagaagagtctgccacctaaaaaggaaataaagatttacttggggctgagtaagatgcaacgagaatggtatacaaaaatcctgatgaaagatattgatgttttaaactcttctggcaagatggacaagatgcgactcttaaacattctgatgcagcttcgaaagtgttgtaatcatccatatctgtttgatggtgctgaacctggtccaccttataccactgatgagcatattgtcagcaacagtggtaaaatggtagttctggataaactattggccaaactcaaagaacagggttcaagggttctcattttcagccagatgactcgcttgctggatattttggaagattattgcatgtggcgtggttatgagtattgtcgactggatggacaaaccccgcatgaagaaagagaggataaattcctagaagtggaatttctgggtcaaagggaagcaatagaggcttttaatgctcctaatagtagcaaattcatctttatgctaagtaccagggctggaggtctcggaattaacctggcaagtgctgatgtggttatactatatgattcagactggaacccacaggttgatctacaagctatggatcgagcacatcgtattggtcagaagaaaccagtacgtgtattccgtctcatcactgacaacactgttgaagagaggattgtagaaagagctgagataaaactgagactcgattcaattgttatacaacaaggaagactcattgaccaacggtctaacaagctggcaaaagaggaaatgttacaaatgatacggcatggagccacccatgtttttgcttctaaagagagtgagttgacagatgaagacattacaactattctggaaagaggggaaaagaagactgcagagatgaatgaacgcctgcaaaaaatgggagagtcttctctaagaaattttagaatggacattgaacaaagtttatacaaatttgagggagaagattatagagaaaaacagaagcttggcatggtggaatggattgaacctcctaaacgagaacgcaaagcaaactacgcagtggatgcctactttagagaggctttgcgtgtcagcgagccaaagattccaaaggctccacggcctccaaaacagccaaatgttcaggattttcaatttttcccaccacgcttatttgagctcctggaaaaggaaattctttattatcggaagacaataggctataaggttccaaggaatcctgatatcccaaatccagctctggctcaaagagaagagcaaaaaaagattgatggagctgaacctcttacaccagaagagactgaagaaaaggaaaaacttctcacacaaggtttcacaaactggactaaacgagattttaaccagtttattaaagctaatgagaaatatggaagagatgacattgataacatagctcgagaggtagagggcaaatcccctgaggaggtcatggagtattcagctgtattttgggaacgttgcaatgaattacaggacattgagaaaattatggctcaaattgaacgtggagaagcaagaattcaacgaaggatcagtatcaagaaagccctggatgccaaaattgcaagatacaaggctccatttcatcagttgcgcattcagtatggaaccagcaaaggaaagaactatactgaggaagaagatagattcttgatttgtatgttacacaaaatgggctttgatagagaaaatgtatatgaagaattaagacagtgtgtacgaaatgctccccagtttagatttgactggtttatcaagtctaggactgccatggaattccagagacgctgtaacactctgatttcattgattgagaaagaaaatatggaaattgaggaaagagagagagcagaaaagaagaaacgggcaactaaaactccaatggtaaaattttcagcattttcctaacttttagatttaacattgttgggccatttaaaatgtgcatattggagcagaacattaaatctgtttccattttagtcacagaaaagaaaagcagagtcagctactgagagctctggaaagaaggatgtcaagaaggtgaaatcctaaagcctagaaataaagttttaaatgggaaactgctattttcttgttcccatcttcaaatgctaattgccagttccagtgtattcatggtactctaagaaaaatctctttggttttgatttcttgcatattttatatattttacaatgctttctacctgaaatgtgtagctttatattttatggcattctagtatttttgtgtactgtattttgtgcatttcatgtcttcatcaaaatcctctcagtccttgttcttttgaagcttgtgctgaggttttagcttttctatgttttatatgccgctgctttgaaagagaacctagattctatagttgtattattgttgtttcatactttaaatttatatggctgtggaaaaacgaattaaaatgttttgaggagaaagaaaaaaaaaa

SEQ ID NO: 135 - DNA, 396 bp, Homo sapiens
atggcggacgtggtcgtgggtaaagacaagggcggggagcagcggctcatctcgctgcctctatcccgcatccgggtcatcatgaagagctcccccgaggtgtccagcatcaaccaggaggcgttggtgctcacggccaaggccacggagctctttgttcaatgcctagccacctattcctacagacacggcagtggaaaggaaaagaaagtactgacttacagtgatttagcaaacactgcacagcaatcagaaacttttcagtttcttgcagatatattaccaaagaagattttagctagtaaatacctgaaaatgcttaaagaggaaaagagggaagaagatgaggagaatgacaatgataatgaaagtgaccatgatgaagctgactcctaa

(19) United States
(12) Patent Application Publication
(10) Pub. No.: US 2005/0202428 A1
(43) Pub. Date: Sep. 15, 2005

(54) Pluripotential stem cells

(75) Inventors: Peter Andrews, Sheffield (GB); James Walsh, Sheffield (GB); Paul Gokhale, Sheffield (GB)

Correspondence Address: CROWELL & MORING LLP;INTELLECTUAL PROPERTY GROUP, P.O. BOX 14300, WASHINGTON, DC, 20044-4300, US

(73) Assignee: AXORDIA LIMITED, SHEFFIELD (GB)

(21) Appl. No.: 10/504,173

(22) PCT Filed: Feb. 12, 2003
(86) PCT No.: PCT/GB03/00579

§ 371 Date: Apr. 22, 2005

(30) Foreign Application Priority Data
Feb. 13, 2002 (GB) 0203359.5
Feb. 13, 2002 (GB) 0203387.6

(51) Int. Cl. C12Q 1/68; C07H 21/02; C12N 5/08; C12N 15/85

(52) U.S. Cl. 435/6; 435/455; 514/44; 435/366; 536/23.1

(57) ABSTRACT

We describe a method to manipulate the phenotype of stem cells, preferably pluripotential stem cells including nucleic acids and vectors used in said methods.

The invention relates to a method to manipulate the phenotype of stem cells, preferably pluripotential stem cells and including nucleic acids and vectors used in said methods.

A number of techniques have been developed in recent years which purport to specifically ablate genes and/or gene products. For example, the use of anti-sense nucleic acid molecules to bind to and thereby block or inactivate target mRNA molecules is an effective means to inhibit the production of gene products. This is typically very effective in plants where anti-sense technology produces a number of striking phenotypic characteristics. However, antisense is variable leading to the need to screen many, sometimes hundreds of, transgenic organisms carrying one or more copies of an antisense transgene to ensure that the phenotype is indeed truly linked to the antisense transgene expression. Antisense techniques, not necessarily involving the production of stable transfectants, have been applied to cells in culture, with variable results.

In addition, the ability to be able to disrupt genes via homologous recombination has provided biologists with a crucial tool in defining developmental pathways in higher organisms. The use of mouse gene “knock out” strains has allowed the dissection of gene function and the probable function of human homologues to the deleted mouse genes, (Jordan and Zant, 1998).

A much more recent technique to specifically ablate gene function is through the introduction of double stranded RNA, also referred to as inhibitory RNA (RNAi), into a cell which results in the destruction of mRNA complementary to the sequence included in the RNAi molecule. The RNAi molecule comprises two complementary strands of RNA (a sense strand and an antisense strand) annealed to each other to form a double stranded RNA molecule. The RNAi molecule is typically derived from exonic or coding sequence of the gene which is to be ablated.

Recent studies suggest that RNAi molecules ranging from 100-1000 bp derived from coding sequence are effective inhibitors of gene expression. Surprisingly, only a few molecules of RNAi are required to block gene expression which implies the mechanism is catalytic. The site of action appears to be nuclear as little if any RNAi is detectable in the cytoplasm of cells indicating that RNAi exerts its effect during mRNA synthesis or processing.

The exact mechanism of RNAi action is unknown although there are theories to explain this phenomenon. For example, all organisms have evolved protective mechanisms to limit the effects of exogenous gene expression. For example, a virus often causes deleterious effects on the organism it infects. Viral gene expression and/or replication therefore needs to be repressed. In addition, the rapid development of genetic transformation and the provision of transgenic plants and animals has led to the realisation that transgenes are also recognised as foreign nucleic acid and subjected to phenomena variously called quelling (Singer and Selker, 1995), gene silencing (Natzke and Matzke, 1998), and co-suppression (Stam et. al., 2000).

Initial studies using RNAi used the nematode Caenorhabditis elegans. RNAi injected into the worm resulted in the disappearance of polypeptides corresponding to the gene sequences comprising the RNAi molecule(Montgomery et. al., 1998; Fire et. al., 1998). More recently the phenomenon of RNAi inhibition has been shown in a number of eukaryotes including, by example and not by way of limitation, plants, trypanosomes (Shi et. al., 2000) Drosophila spp. (Kennerdell and Carthew, 2000). Recent experiments have shown that RNAi may also function in higher eukaryotes. For example, it has been shown that RNAi can ablate c-mos in a mouse ooctye and also E-cadherin in a mouse preimplanation embryo (Wianny and Zernicka-Goetz, 2000).

During mammalian development those cells that form part of the embryo up until the formation of the blastocyst are said to be totipotent (e.g. each cell has the developmental potential to form a complete embryo and all the cells required to support the growth and development of said embryo). During the formation of the blastocyst, the cells that comprise the inner cell mass are said to be pluripotential (e.g. each cell has the developmental potential to form a variety of tissues).

Embryonic stem cells (ES cells, those with pluripotentiality) may be principally derived from two embryonic sources. Cells isolated from the inner cell mass are termed embryonic stem (ES) cells. In the laboratory mouse, similar cells can be derived from the culture of primordial germ cells isolated from the mesenteries or genital ridges of days 8.5-12.5 post coitum embryos. These would ultimately differentiate into germ cells and are referred to as embryonic germ cells (EG cells). Each of these types of pluripotential cell has a similar developmental potential with respect to differentiation into alternate cell types, but possible differences in behaviour (eg with respect to imprinting) have led to these cells to be distinguished from one another.

Typically ES/EG cell cultures have well defined characteristics. These include, but are not limited to;
i) maintenance in culture for at least 20 passages when maintained on fibroblast feeder layers;
ii) produce clusters of cells in culture referred to as embryoid bodies;
iii) ability to differentiate into multiple cell types in monolayer culture;
iv) can form embryo chimeras when mixed with an embryo host;
v) express ES/EG cell specific markers.

Until very recently, in vitro culture of human ES/EG cells was not possible. The first indication that conditions may be determined which could allow the establishment of human ES/EG cells in culture is described in WO96/22362. The application describes cell lines and growth conditions which allow the continuous proliferation of primate ES cells which exhibit a range of characteristics or markers which are associated with stem cells having pluripotent characteristics.

More recently Thomson et al (1998) have published conditions in which human ES cells can be established in culture. The above characteristics shown by primate ES cells are also shown by the human ES cell lines. In addition the human cell lines show high levels of telomerase activity, a characteristic of cells which have the ability to divide continuously in culture in an undifferentiated state. Another group (Reubinoff et. al., 2000) have also reported the derivation of human ES cells from human blastocyts. A third group (Shamblott et. al., 1998) have described EG cell derivation.

A feature of ES/EG cells is that, in the presence of fibroblast feeder layers, they retain the ability to divide in an undifferentiated state for several generations. If the feeder layers are removed then the cells differentiate. The differentiation is often to neurones or muscle cells but the exact mechanism by which this occurs and its control remain unsolved.

In addition to ES/EG cells a number of adult tissues contain cells with stem cell characteristics. Typically these cells, although retaining the ability to differentiate into different cell types, do not have the pluripotential characteristics of ES/EG cells. For example haemopoietic stem cells have the potential to form all the cells of the haemopoietic system (red blood cells, macrophages, basophils, eosinophils etc). All of nerve tissue, skin and muscle retain pools of cells with stem cell potential. Therefore, in addition to the use of embryonic stem cells in developmental biology, there are also adult stem cells which may also have utility with respect to determining the factors which govern cell differentiation. Further recent studies have suggested that some stem cells previously thought to be committed to a single fate, (e.g neurons) may indeed possess considerable pluripotentcy in certain situations. Neural stem cells have recently been shown to chimerise a mouse embryo and form a wide range of non-neural tissue (Clark et. al., 2000).

A further group of cells which have relevance to developmental biology are teratocarcinoma cells (EC cells). These cells form tumours referred to as teratomas and have many features in common with ES/EG cells. The most important of these features is the characteristic of pluripotentiality.

Teratomas contain a wide range of differentiated tissues, and have been known in humans for many hundreds of years. They typically occur as gonadal tumours of both men and women. The gonadal forms of these tumours are generally believed to originate from germ cells, and the extra gonadal forms, which typically have the same range of tissues, are thought to arise from germ cells that have migrated incorrectly during embryogenesis. Teratomas are therefore generally classed as germ cell tumours which encompasses a number of different types of cancer. These include seminoma, embryonal carcinoma, yolk sac carcinoma and choriocarcinoma

The similar biology of EC cells with ES/EG cells has been exploited to study the developmental fates of cells and to identify cell markers commonly expressed in EC cells and ES/EG cells. For example, and not by way of limitation, the expression of specific cell surface markers SSEA-3 (+), SSEAM (+), TRA-1-60 (+), TRA-1-81 (+) (Shevinsky et al 1982; Kannagi et al 1983; Andrews et al 1984a; Thomson et al 1995); alkaline phosphatase (+) (Andrews et. al., 1996); and Oct 4 (Scholer et. al., 1989; Kraft et. al., 1996; Reubinoff et. al., 2000; Yeom et. al., 1996).

It is well known that gene expression can be affected at many levels. For example, at the level of transcription, translation or post-translationally by modifications to proteins which confer an altered biological activity to the modified protein. It is also known that the way in which DNA is packaged as chromatin can influence the expression of genes.

There are several levels of structural packaging of DNA leading from a double stranded helix to a mitotic chromosome, after which the DNA is some 50,000 times shorter than its extended length (Alberts et al., 1998). Double-stranded helical DNA is wound around the structural unit of a nucleosome, comprising an octamer core composed of 4 types of histones: two each of the H2A, H2B, H3, and H4 proteins. Approximately 166 base pairs are bound to the nucleosome through electrostatic forces between the negatively charged phosphate groups in the DNA backbone and positively charged amino acids (e.g., lysine and arginine) in the histone proteins (Wolfe, 1993). Whilst the majority of the base pairs are tightly bound to the octamer core, the remaining linker DNA (80-100 bp) hat separates adjacent core particles is associated with the H1 histone or a related “linker” histone (Finch and Klug, 1976; Thoma et al., 1979; Wolfe, 1993).

Nucleosomes are organised into the next structural level of the chromatin fibre, also referred to as a solenoid. Chromatin structure is not static and the regulated alteration in structure is termed ‘chromatin remodelling’. This process has been defined as any event that alters the nuclease sensitivity of a region of chromatin, and can occur independently or in concert with processes such as transcription (Aalfs & Kingston, 2000). For a comprehensive review of chromatin remodelling see Aalfs & Kingston, 2000.

Reversible acetylation of evolutionary conserved lysine residues in core histone proteins plays a critical role in transcriptional regulation, cell cycle progression, and developmental events. The steady state of histone acetylation is controlled by the enzymatic activities of multiple histone acetyltransferases (HATs) and histone deacetylases (HDACs). Histone hyperacetylation is associated with transcriptional activity while histone hypoacetylation correlates with transcriptional quiescence and so histone deacetylases can be considered as enzymatic transcriptional repressors. Histone deacetylases were first described by Inove & Fujimoto, 1969.

In general, histone deacetylases do not target genes directly through specific DNA-binding sites. Rather, deacetylases are localized to genes targeted for repression as part of a protein complex. Other proteins that are part of this complex, termed co-repressors, are responsible for targeting the genes to be repressed. A large number of such co-repressors have been identified to date, including the thyroid hormone receptor, Sin3, SMRT, mYY1, and MeCP2, for a comprehensive review see Pazin & Kadonaga, 1997.

In humans, four highly homologous class I HDAC enzymes (HDAC1, HDAC2, HDAC3, and HDAC8) have been identified to date, with HDAC1, HDAC2 and HDAC3 being ubiquitously expressed in many different cell types (Yang et al., 1997 and 2002). HDAC1 and HDAC2 are the human orthologues of the yeast transcriptional regulator RPD3. Analysis of the predicted amino acid sequence of HDAC3 revealed an open reading frame of 428 amino acids with a predicted molecular mass of 49 kDa.

The HDAC3 protein is 50% identical in DNA sequence and 53% identical in protein sequence compared with the previously cloned human HDAC1. Comparison of the HDAC3 sequence with human HDAC2 also yielded similar results, with 51% identity in DNA sequence and 52% identity in protein sequence (Yang et al., 1997). The expressed HDAC3 protein is functionally active because it possesses histone deacetylase activity, represses transcription when tethered to a promoter, and binds transcription factor YY1. Although HDAC3 shares some structural and functional similarities with other class I HDACs, it exists in multi-subunit complexes separate and different from other known HDAC complexes, implying that individual HDACs might function in a distinct manner (Yang et al., 2002). Within the HDACs there are three regions of highly conserved amino acid residues; histidines, aspartates and glycines, irrespective of the highly divergent nature of the C-terminal regions (Hassig et al., 1998). It is presumed that these regions form part of the active site and are also involved in maintaining interactions between [DACs and members of the co-repressor complex.

In Drosophila, active and silent states of developmentally regulated loci are maintained by trithorax and Polycomb group of proteins. Proteins of the polycomb and trithorax groups act to remodel chromatin by altering the accessibility of DNA to factors required for gene transcription. The PcG proteins are required to maintain the transcriptionally inactive state, whereas the trxG proteins are necessary to counteract silencing and maintain the transcriptionally active state. Both PcG and trxG proteins are thought to function by establishing closed or open chromatin configurations at their target genes

In humans, homologues of the members of the Drosopila polycomb group (Pc-G) proteins include; the YY1 transciption factor (YY1), the chromobox 2 gene (CBX1) and the PHD finger protein 1, transcript variant 2 (BF1) gene. Pc-G proteins are usually considered to be inhibitors of homeotic genes. Pc-G mutants were originally identified on the basis of their causing expression of homeotic genes in unusual (ectopic) locations. This ectopic expression of genes was attributed to the failure of proper gene silencing. Pc-G proteins themselves are unable to bind to DNA, their action is dependent on their association with other chromosomal proteins, especially histones. It is suggested that the initial repression of a gene is carried out by transcription factors which have the ability to recognize DNA and that Pc-G proteins then provide a mechanism where this initial repression becomes permanent by assembling at this site and forming a multiprotein complex involved in modifying chromatin.

In humans, homologues of the members of the Drosophila Trithorax (IRX) proteins, for example an enhancer of polycomb 1 (EPC1), a zinc finger protein 144 (WEL18) and a myeloid/lymphoid or mixed lineage leukemia 1 (ULLT1) are considered to be activators of homeobox genes. Mutations within trx genes result in transformations of body structures reminiscent of loss-of-function mutations in homeotic genes. For example, in the Drospholia, after the disappearance of the transiently acting patterning factors such as those encoded by the segmentation genes, maintenance of the initial transcriptional patterns of homeotic genes requires the expression of the trx gene. (Orlando et al., 1998). Genetic analyses indicate that trx expression is required continuously throughout Drosophila development, consistent with its maintenance function, but there also appears to be a critical early requirement, which if compromised cannot be compensated by subsequent continuous expression (Ingham and Whittle, 1980). Comparatively little is known about the molecular environment in which the TRX protein is integrated. However, an elucidation of this issue could be particularly rewarding as chromosomal aberrations involving the human homologues of TRX (L, ALL-1, HRX) is one of the most frequent genetic changes in infant leukemias of myeloid and lymphoid lineage and in treatment-induced secondary leukemias (Orlando et al., 1998).

Whereas some multi-protein complexes which alter transcriptional regulation and chromatin remodelling are based on the covalent modification of the histones (e.g. histone acetyltransferases (HATs) and histone deacetylases, others appear to function by altering chromatin structure in an ATP dependent fashion (e.g. the yeast SWI/SNF complex). A group of enzymes referred to as ATP-dependent chromatin remodellers, use the energy of ATP hydrolysis to alter interactions between DNA and histone proteins. The protein complexes that mediate ATP-dependent nucleosome remodelling and histone acetylation/deacetylation in the regulation of transcription were initially considered to be different, although it has recently been suggested that their activities might be coupled. Examples of human ATP-dependent chromatin remodellers include, SMARCA5, a human SWI/SNF related, matrix associated and actin dependent regulator of chromatin, identified as member 5 of subfamily ‘a’ of the SMARC family.

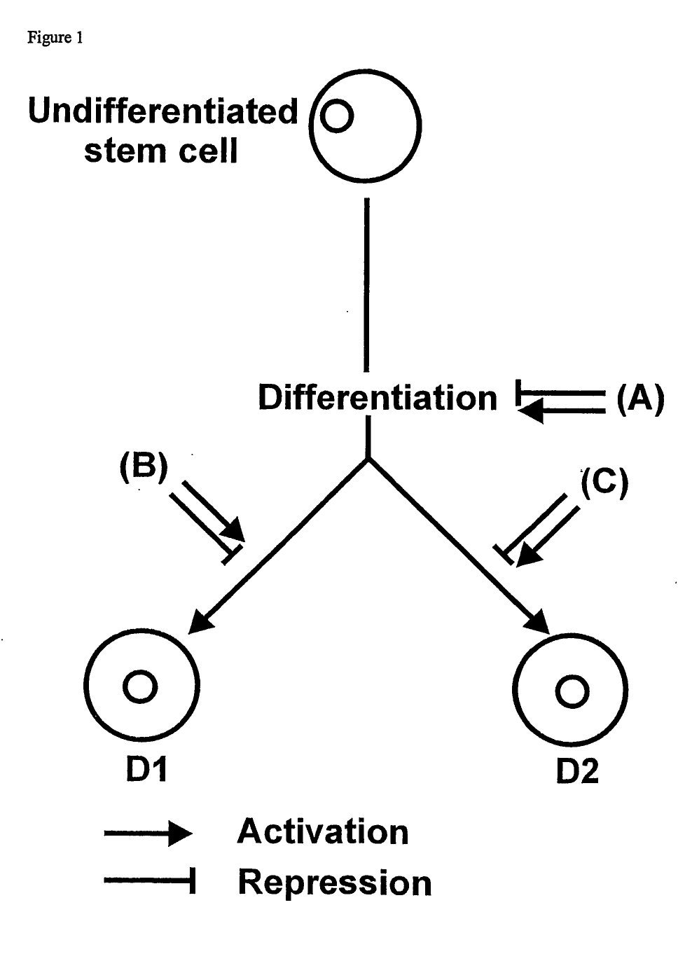

There are further examples of regulators of gene expression. We have accumulated expression studies which identify a number of genes thought to be involved in determining the developmental fate of stem cells, particularly embryonic stem cells. By northern blotting we have identified the expression of human homologs of two signalling pathways believed to be critical in cell fate determination. Expression of ligands, receptors and downstream components of the Notch and Wingless signalling cascades have been elucidated. Using the model system NTERA2/D1 embryonal carcinoma cells we have recorded changes in the expression of some of these components as the cells differentiate. Bearing in mind the role these cascades play in embryonic development throughout the animal kingdom, these changes suggest a significant role for both the wingless and Notch signalling pathways in differentiation of stem cells. Furthermore the activity of some genes are required for differentiation to occur along specific pathways e.g. the myogenic gene MyoD1. Other genes have activity which inhibits cellular differentiation along particular pathways. We envisage regulation of stem cell differentiation to yield a specific cell type could be achieved by.
(i) inhibition of certain genes that normally promote differentiation along particular pathways; therefore promoting differentiation to alternate cell phenotypes;
(ii) inhibition of gene activity that prevents differentiation into particular cell types; and
(iii) a combination of (i) and (ii), see FIG. 1

The differentiation of stem cells during embryogenesis, during tissue renewal in the adult and wound repair is under very stringent regulation: aberrations in this regulation underlie the formation of birth defects during development and are thought to underlie cancer formation in adults. Generally, it is envisaged that such stem cells are under both positive and negative regulation which allows a fine degree of control over the process of cell proliferation and cell differentiation: excess proliferation at the expense of cell differentiation can lead to the formation of an expanding mass of tissue—a cancer—whereas express differentiation at the expense of proliferation can lead to the loss of stem cells and production of too little differentiated tissue in the long term, and especially the loss of regenerative potential. Certain genes have already been identified to have a negative role in preventing stem cell differentiation. Such genes, like those of the Notch family, when mutated to acquire activity can inhibit differentiation; such mutant genes act as oncogenes. On the contrary, loss of function of such genes on their inhibition results in stem cell differentiation. We propose to use EC cells as our model cell system to follow the effects of RNAi on cell fate.

In our co-pending application, WO02/16620, discloses RNAi molecules derived from the following nucleic acid sequences which encode the following polypeptides; human Notch 1(hNotch); hNotch 2; hNotch 3; hNotch 4; TLE-1; TLE-2; TLE-3; TLE-4; TCF7; TCF7L1; TCFFL2; TCF3; TCF19; TCF1; mfringe; lFringe; rFringe; sel 1; Numb; Numblike; LNX; FZD1; FZD2; FZD3; FZD4; FZD5; FZD6; FZD7; FZD8; FZD9; FZD10; FRZB, D11-1; D113; D114; Dlk-1; Jagged 1; Jagged 2; Wnt 1; Wnt 2; Wnt 2b; Wnt 3; Wnt 3a; Wnt5a; Wnt6; Wnt7a; Wnt7b; Wnt8a; Wnt8b; Wnt10b; Wnt11; Wnt14; Wnt15, SFRP1; SFRP2; SFRP4; SFRP5; SK; DKK3; CER1; WIF-1; DVL1; DVL2; DVL3; DVL1L1;mFringe; lFringe; rFringe; selll; Numb; INX Oct4; NeuroD1; NeuroD2; NeuroD3; Brachyury; MDFI, CIR, DLK1; Oct 4; RBPJk. The present application disclaims these genes the sequences of which are disclosed in WO02/16620.

One further family of genes are the BES and related genes which are direct targets of Notch signaling. Binding of Notch ligands to Notch receptor causes proteolytic cleavage of the receptor (Mumm and Kopan, 2000). The cleaved receptor known as Notch-intracellular domain (NICD) translocates to the nucleus and binds to RBP-Jr,. This binding changes RBP-Jκ from a repressor to an activator of its target genes. The target genes are homologs of the genes found at the Drosophila Enhancer of Split complex (E(spl)). These basic helix-loop-helix (bHLH) transcription factors act as repressors of downstream tissue specific transcription factors and as such act as notch effectors. The Notch signaling through E(spl) complex genes represses certain tissue specific transcription factors.

The E(spl) family of proteins are class three bHLH factors. These include: HES1, BES2, HES4, HES6, HES7, HERP1, BERP2, BESR1, HBY1, BEY2, HEYL, HRT1, HRT2, HRT3 CBF1, CHF2 GRIDLOCK.

The various members of the HES related genes encode proteins that are homologous in key motif regions. They all contain Basic helix-loop-helix and a so called orange domain. HES family members contain a terminal WRPW domain and HEY family proteins contain YRPW or closely related residues. FIG. 1 shows an alignment of human BES related proteins illustrating the major domains contained in the IES related proteins. ES/EC differentiation go through a precursor stage for example neural differentiation (rzyborski et. al., 2001) during differentiation to the numerous lineages that can form in vitro.

Notch signaling through E(spl) homologs possibly allows precursor cells to remain as precursors. In addition Notch may also play an instructive role in specifying cell types, for example (Hojo et. al., 2000). Manipulation of the E(spl) homologs and other downstream targets which directly affect these processes would alter the notch signaling in target cells. This in turn would alter the balance between cells types. This could be manipulated to for example block a particular cell type forming by stopping the instructive signaling or by increasing or removing the precursor cells from the cultures. The E(spl) complex genes are potential targets which would allow cell type specific disruption of Notch signaling in differentiating cultures of stem cells.

According to an aspect of the invention there is provided a method to modulate the differentiation state of a stem cell comprising the steps of:
i) contacting a stem cell with at least one inhibitory RNA molecule (RNAi) comprising a sequence of a gene which mediates at least one step in the differentiation of said cell;
(ii) providing conditions conducive to the proliferation of the cell treated in (i) above; and optionally
(iii) maintaining and/or storing said cell.

The term modulate includes both promoting or inducing the differentiation of a stem cell into a lineage restricted stem cell or a differentiated cell or to maintain a stem cell as a stem cell with characteristics which are typical of stem cells, particularly embryonic stem cells. For example, maintenance in culture for at least 20 passages when maintained on fibroblast feeder layers; production of embryoid bodies in culture; the ability to differentiate into multiple cell types in monolayer culture; can form embryo chimeras when mixed with an embryo host; and express ES/EG cell specific markers.

In a preferred method of the invention said method is an in vitro method.

In an alternative preferred method said method is an in vivo method.

In a further preferred method of the invention said stem cell is selected from the group consisting of: haemopoietic stem cells; neural stem cells; bone stem cells; muscle stem cells; mesenchymal stem cells; trophoblastic stem cells; epithelial stem cells (derived from organs such as the skin, gastrointestinal mucosa, kidney, bladder, mammary glands, uterus, prostate and endocrine glands such as the pituitary); endodermal stem cells (derived from organs such as the liver, pancreas, lung and blood vessels); embryonic stem (ES) cells; embryonal germ (EG) cells.

In a further preferred method of the invention said stem cells are embryonal carcinoma cells. Preferably said embryonal carcinoma cells are TERA2 cells. Ideally said embyonal carcinoma cells are NTERA 2 cells.

In a further preferred method of the invention said stem cell is an embryonic stem cell or embryonal germ cell or an embryonal carcinoma cell.

In a preferred method of the invention said gene is involved in Notch/Wnt signalling.

In a preferred method of the invention said RNAi molecule is derived from a nucleic acid molecule comprising a nucleic acid sequence selected from the group consisting of:
i) a nucleic acid sequence as represented by table 1, or fragment thereof;
ii) a nucleic acid sequence which hybridises to the nucleic acid sequence of table 1 and which modulates stem cell differentiation;
iii) a nucleic acid sequence which comprise sequences which are degenerate as a result of the genetic code to the nucleic acid sequences defined in (i) and (ii).

In a preferred method of the invention said hybridisation conditions are stringent hybridisation conditions.

Typically, hybridisation conditions uses 4-6×SSPE (20×SSPE contains 175.3 g NaCl, 88.2 g NaH₂PO₄ H₂O and 7.4 g EDTA dissolved to 1 litre and the pH adjusted to 7.4); 5-10× Denhardts solution (50× Denhardts solution contains 5 g Ficoll (type 400, Pharmacia), 5 g polyvinylpyrrolidone abd 5 g bovine serum albumen; 100 μg-1.0 mg/ml sonicated salmon/herring DNA; 0.1-1.0% sodium dodecyl sulphate; optionally 40-60% deionised formamide. Hybridisation temperature will vary depending on the GC content of the nucleic acid target sequence but will typically be between 42°-65°. It is well known in the art that optimal hybridisation conditions can be calculated if the sequences of the nucleic acid is known. For example, hybridisation conditions can be determined by the GC content of the nucleic acid subject to hybridisation. Please see Sambrook et al (1989) Molecular Cloning; A Laboratory Approach. A common formula for calculating the stringency conditions required to achieve hybridisation between nucleic acid molecules of a specified homology is:
Tₘ=81.5° C.+16.6 Log[Na⁺]+0.41[% G+C]−0.63 (% formamide).

In a preferred method of the invention said RNAi molecule is derived from a nucleic acid sequence encoding a Notch receptor processing factor polypeptide selected from the group consisting of: Nrarp; P300; presenilin associated protein; presenilin 1; presenilin 2; or Sel-1.

In an alternative preferred method of the invention said RNAi molecule is derived from a nucleic acid molecule encoding a Notch target gene selected from the group consisting of: HERP1; HERP2; HES1; HES 2; HES 4; HES6, HES7, HERP1, HERP2, HESR1, HEY1, HEY2, HEYL HRT1, HRT2, HRT3 CBF1, CHF2 GRIDLOCK.

In a preferred method of the invention said RNAi molecule is derived from a nucleic acid molecule comprising a nucleic acid sequence selected from the group consisting of:
i) a nucleic acid sequence as represented by the sequences in SEQ ID NO: 7-23, or fragment thereof;
ii) a nucleic acid sequence which hybridises to the nucleic acid sequences of SEQ ID NO: 7-23 and is a Notch-signalling target gene;
iii) a nucleic acid sequence which comprise sequences which are degenerate as a result of the genetic code to the nucleic acid sequences defined in (i) and (ii).

In a further alternative preferred method of the invention said RNAi molecule is derived from a nucleic acid molecule encoding a Wnt ligand processing factor selected from the group consisting of: LRP1; LRP2; LRP3; LRP4; LRP5; LRP6; LRP8; or Porcupine.

Alternatively said RNAi molecule is derived from a nucleic acid molecule encoding an extracellular Wnt antagonist selected from the group consisting of: Dkl1; Dkk2; Dkk3; Dkk4; Frzb; or SARP1.

In a further preferred method of the invention said RNAi molecule is derived from a nucleic acid molecule encoding a Wnt cytoplasmic acting component selected from the group consisting of: APC; Axin1; Axin2; FRAT1; GSK3; ICAT; IDAX; Par 1; or TAB1.

Alternatively, said RNAi molecule is derived from a nucleic acid molecule encoding a Wnt nuclear acting component selected from the group consisting of: β-catenin; β-TRCP; CBP; CTBP1; HBP-1; Lef1; NIX; Pontin 52; Reptin 52.

In a yet further alternative method of the invention said RNAi molecule is derived from a nucleic acid molecule which encodes a Wnt target gene selected from ASCL 1 or ASCL 2.

In a yet still further preferred method of the invention said RNAi molecule is derived from a nucleic acid molecule selected from the group consisting of: FGF 5; msx 1; neurogenin 1; neurogenin 2; neurogenin 3; or PTEN.

In an alternative preferred method of the invention said RNAi molecule is derived from a gene which encodes a polypeptide involved in modifying chromatin conformation.

In a preferred method of the invention said RNAi molecule is derived from a nucleic acid sequence which encodes a polypeptide which modifies a histone polypeptide. Preferably said histone modifying polypeptide is a histone deacetylase. Preferably said RNAi is derived from a mammalian class I histone deacetylase.

In a preferred method of the invention said nucleic acid molecule comprises a nucleic acid sequence selected from the group consisting of:
i) a nucleic acid sequence as represented by the sequences in Table 4, or fragment thereof;
ii) a nucleic acid sequence which hybridises to the nucleic acid sequences of Table 4 and which has histone deacetylase activity;
iii) a nucleic acid sequence which comprise sequences which are degenerate as a result of the genetic code to the nucleic acid sequences defined in (i) and (ii).

Preferably said histone deacetylase is selected from the group consisting of: HDAC1; HDAC2; HDAC3; HDAC 4; HDAC5; HDAC6; HDAC7; IDAC8; hSIRT2; hSIRT3; hSIRT4; hSIRT5; hSIRT6; hSIRT7; MECP2; ZNF145; TPDP1; SAP30; SAP18; RBBP7; RBBP4; RB1;MEN1.

Alternatively, said histone modifing polypeptide is a histone acetyltransferase selected from the group consisting of:
i) a nucleic acid sequence as represented by the sequences in Table 5, or fragment thereof;
ii) a nucleic acid sequence which hybridises to the nucleic acid sequences of Table 5 and which has histone acetyltransferase activity,
iii) a nucleic acid sequence which comprise sequences which are degenerate as a result of the genetic code to the nucleic acid sequences defined in (i) and (ii).

In a preferred method of the invention said histone acetyltransferase is selected from the group consisting of: Gcn 5; Gcn5L2; PCAF; MOZ; HBO; CBP; SCR-1; pGRIP; ATF-2; and HAT1.

In a further preferred method of the invention said RNAi molecule comprises a nucleic acid sequence derived from a gene selected from the group consisting of:
i) a nucleic acid sequence as represented by the sequences in Table 2, or fragment thereof;
ii) a nucleic acid sequence which hybridises to the nucleic acid sequences of Table 2 and which mediates chromatin conformation;
iii) a nucleic acid sequence which comprise sequences which are degenerate as a result of the genetic code to the nucleic acid sequences defined in (i) and (ii).

In a preferred method of the invention said nucleic acid encodes a polypeptide which mediates chromatin conformation selected from the group consisting of: EED; YY1; C13X1; CBX6; HPC2(CBX4); BPC3(CBX8); PHF1; PHM2; HPH1; HPH2; SSX1; and SSX2.

In a further preferred method of the invention said RNAi molecule comprises a nucleic acid sequence derived from a gene selected from the group consisting of:
i) a nucleic acid sequence as represented by the sequences in Table 3, or fragment thereof;
ii) a nucleic acid sequence which hybridises to the nucleic acid sequences of Table 3 and which mediates chromatin conformation;
iii) a nucleic acid sequence which comprise sequences which are degenerate as a result of the genetic code to the nucleic acid sequences defined in (i) and (id).

In a preferred method of the invention said nucleic acid encodes a polypeptide which mediates chromatin conformation selected from the group consisting of: EPC1; EZH1; EZH2; BMI1; MEL18; SCML1; SCML2; RING1; RYBP; MLL; MLLT1; MLLT7; MLLT6; MLLT4; MLLT3; MLLT2; MLLT10; and MLL2.

In a further preferred method of the invention said RNAi molecule comprises a nucleic acid sequence derived from a gene selected from the group consisting of:
i) a nucleic acid sequence as represented by the sequences in Table 6, or fragment thereof;
ii) a nucleic acid sequence which hybridises to the nucleic acid sequences of Table 6 and which mediates chromatin conformation;
iii) a nucleic acid sequence which comprise sequences which are degenerate as a result of the genetic code to the nucleic acid sequences defined in (i) and (ii).

In a preferred method of the invention said nucleic acid encodes a polypeptide which mediates chromatin conformation selected from the group consisting of: SMARCA 5; SMARCA 2; SMARCA 4; SMARCA 3; SMARCAL1; SMARCA 1; and CHRAC1.

In a preferred method of the invention said RNAi molecule comprises a first part linked to a second part wherein said first and second parts are complementary over at least part of their length and further wherein said first and second parts form a double stranded region by complementary base pairing over at least part of their length.

The provision of first and second sequences which are complementary to one another and which comprise at least part of the coding sequence of a gene involved in stem cell differentiation means that when the sequence is transcribed into RNA the complementarity between first and second sequences allows base pairing between first and second sequences to form a double stranded RNA structure. The optional provision of a linking region bewteen first and second parts results in the formation of a so called “hair-pin” loop structure. The transcription of the nucleic acid provides many copies of the hair-pin loop RNA which effectively functions as a RNAi molecule. The hair-pin loop RNA can be transcribed in vitro using, for example commercially available transcription kits which utilise phage RNA polymerase or in vivo using vectors adapted for expression by a cell, typically a eukaryotic cell, preferably a lineage restricted stem cell or embryonic stem cell.

According to a further aspect of the invention there is provided an RNAi molecule which comprises a sequence of a gene wherein said gene mediates stem cell differentiation.

In a preferred embodiment of the invention said RNAi molecule comprises a first part linked to a second part wherein said first and second parts are complementary over at least part of their length and further wherein said first and second parts form a double stranded region by complementary base pairing over at least part of their length.

In a further preferred embodiment of the invention said first and second parts are linked by at least one nucleotide base. In a further preferred embodiment of the invention said first and second parts are linked by 2, 3, 4, 5, 6, 7, 8, 9, or 10 nucleotide bases. In a yet further preferred embodiment of the invention said linker is at least 10 nucleotide bases.

In a further preferred embodiment said coding sequence is an exon.

Alternatively said RNA molecule is derived from intronic sequences or the 5′ and/or 3′ non-coding sequences which flank coding/exon sequences of genes which modulate stem cell differentiation.

In a further preferred embodiment of the invention the length of the RNAi molecule is between 10 nucleotide bases (nb)-1000nb. More preferably still the length of the RNA molecule is selected from 10nb; 20nb; 30nb; 40nb; 50nb; 60nb; 70nb; 80nb; 90nb. More preferably still said RNA molecule is 21nb in length. Preferably said RNAi molecule comprises 19 complementary bases with a 3′ 2nb overhang at either end.

In a further preferred embodiment of the invention said RNA molecule is 100nb; 200nb; 300nb; 400nb; 500nb; 600nb; 700nb; 800nb; 900nb; or 1000nb. More preferably still said RNA molecule is at least 1000nb.

In yet a further preferred embodiment of the invention said RNAi molecules comprise modified nucleotide bases.

It will be apparent to one skilled in the art that the inclusion of modified bases, as well as the naturally occuring bases cytosine, uracil, adenosine and guanosine, may confer advantageous properties on RNAi molecules containing said modified bases. For example, modified bases may increase the stability of the RNAi molecule thereby reducing the amount required to produce a desired effect. The provision of modified bases may also provide RNAi molecules which are more or less stable.

The term “modified nucleotide base” encompasses nucleotides with a covalently modified base and/or sugar. For example, modified nucleotides include nucleotides having sugars which are covalently attached to low molecular weight organic groups other than a hydroxyl group at the 3′ position and other than a phosphate group at the 5′ position. Thus modified nucleotides may also include 2′ substituted sugars such as 2′-O-methyl-; 2-O-alkyl; 2-O-allyl; 2′-S-alkyl; 2′-S-allyl; 2′- fluoro-; 2′-halo or 2;azido-ribose, carbocyclic sugar analogues a-anomeric sugars; epimeric sugars such as arabinose, xyloses or lyxoses, pyranose sugars, furanose sugars, and sedoheptulose.

Modified nucleotides are known in the art and include by example and not by way of limitation; alkylated purines and/or pyrimidines; acylated purines and/or pyrimidines; or other heterocycles. These classes of pyrimidines and purines are known in the art and include, pseudoisocytosine; N4, N4-ethanocytosine; 8-hydroxy-N6-methyladenine; 4-acetylcytosine, 5-(carboxyhydroxylmethyl) uracil; 5-fluorouracil; 5-bromouracil; 5-carboxymethylaminomethyl-2-thiouracil; 5-carboxymethylaminomethyl uracil; dihydrouracil; inosine; N6-isopentyl-adenine; 1-methyladenine; 1-methylpseudouracil; 1-methylguanine; 2,2-dimethylguanine; 2-methyladenine; 2-methylguanine; 3-methylcytosine; 5-methylcytosine; N6-methyladenine; 7-methylguanine; 5-methylaminomethyl uracil; 5-methoxy amino methyl-2-thiouracil; β-D-mannosylqueosine; 5-methoxycarbonylmethyluracil; 5-methoxyuracil; 2 methylthio-N6-isopentenyladenine; uracil-5-oxyacetic acid methyl ester; psueouracil; 2-thiocytosine; 5-methyl-2 thiouracil, 2-thiouracil; 4-thiouracil; 5-methyluracil; N-uracil-5-oxyacetic acid methylester; uracil 5-oxyacetic acid; queosine; 2-thiocytosine; 5-propyluracil; 5-propylcytosine; 5-ethyluracil; 5-ethylcytosine; 5-butyluracil; 5-pentyluracil; 5-pentylcytosine; and 2,6,-diaminopurine; methylpsuedouracil; 1-methylguanine; 1-methylcytosine;

The RNAi molecules of the invention can be synthesized using conventional phosphodiester linked nucleotides and synthesized using standard solid or solution phase synthesis techniques which are known in the art. Linkages between nucleotides may use alternative linking molecules. For example, linking groups of the formula P(O)S, (thioate); P(S)S, (dithioate); P(O)NR′2; P(O)R′; P(O)OR6; CO; or CONR′2 wherein R is H (or a salt) or alkyl (1-12C) and R6 is alkyl (1-9C) is joined to adjacent nucleotides through —O— or —S—.

According to a further aspect of the invention there is provided a nucleic acid molecule encoding at least part of a gene which modulates stem cell differentiation comprising a first part linked to a second part which first and second parts are complementary over at least part of their length, wherein said nucleic acid molecule is operably linked to at least one further nucleic acid molecule capable of promoting transcription of said nucleic acid linked thereto and further wherein said first and second parts form a double stranded region by complementary base pairing over at least part of their length as or when said nucleic acid molecule is transcribed.

In a preferred embodiment of the invention said first and second parts are linked by linking nucleotides as hereinbefore described.

It will be apparent to one skilled in the art that the synthesis of RNA molecules which form RNA stem loops can be achieved by providing vectors which include target genes, or fragments of target genes, operably linked to promoter sequences. Typically, promoter sequences are phage RNA polymerase promoters (eg T7, T3, SP6). Advantageously vectors are provided with multiple cloning sites into which genes or gene fragments can be subcloned. Typically, vectors are engineered so that phage promoters flank multiple cloning sites containing the gene of interest.

Alternatively target genes or fragments of target genes can be fused directly to phage promoters by creating chimeric promoter/gene fusions via oligo synthesising technology. Constructs thus created can be easily amplified by polymerase chain reaction to provide templates for the manufacture of RNA molecules comprising stem loop RNA's.

According to a further aspect of the invention there is provided an expression vector including an expression cassette comprising at least one nucleic acid molecule encoding an RNAi molecule according to the invention.

Vectors including expression cassettes encoding stem-loop RNA's are adapted for eukaryotic gene expression. Typically said adaptation includes, by example and not by way of limitation, the provision of transcription control sequences (promoter sequences) which mediate cell/tissue specific expression. These promoter sequences may be cell/tissue specific, inducible or constitutive.

Promoter elements typically also include so called TATA box and RNA polymerase initiation selection sequences which function to select a site of transcription initiation. These sequences also bind polypeptides which function, inter alia, to facilitate transcription initiation selection by RNA polymerase.

Adaptations also include the provision of selectable markers and autonomous replication sequences which both facilitate the maintenance of said vector in either the eukaryotic cell or prokaryotic host. Vectors which are maintained autonomously are referred to as episomal vectors. Further adaptations which facilitate the expression of vector encoded genes include the provision of transcription termination sequences.

These adaptations are well known in the art. There is a significant amount of published literature with respect to expression vector construction and recombinant DNA techniques in general. Please see, Sambrook et al (1989) Molecular Cloning: A Laboratory Manual, Cold Spring Harbour Laboratory, Cold Spring Harbour, N.Y. and references therein; Marston, F (1987) DNA Cloning Techniques: A Practical Approach Vol III IRL Press, Oxford UK; DNA Cloning: F M Ausubel et al, Current Protocols in Molecular Biology, John Wiley & Sons, Inc.(1994).

In a preferred embodiment of the invention said RNAi molecule is derived from a nucleic acid molecule encoding a notch receptor processing factor polypeptide selected from the group consisting of: Nrarp; P300; presenilin associated protein; presenilin 1; presenilin 2; or Sel-1.

In an alternative preferred embodiment said RNAi molecule is derived from a nucleic acid molecule encoding a Notch target gene selected from the group consisting of: HBRP1; HBERP2; HES1; HES 2; HES 4; HES6, HES7, HERP1, HERP2, HESR1, HEY1, HEY2, HEYL HRT1, HRT2, HRT3 CHF1, CHF2 GRIDLOCK.

In a preferred embodiment of the invention said RNAi molecule is derived from a nucleic acid molecule comprising a nucleic acid sequence selected from the group consisting of:
i) a nucleic acid sequence as represented by the sequences in SEQ ID NO: 7-23, or fragment thereof;
ii) a nucleic acid sequence which hybridises to the nucleic acid sequences of SEQ ID NO: 7-23 and is a Notch signalling target gene;
iii) a nucleic acid sequence which comprise sequences which are degenerate as a result of the genetic code to the nucleic acid sequences defined in (i) and (ii).

In a further alternative method of the invention said RNAi molecule is derived from a nucleic acid molecule encoding a Wnt ligand processing factor selected from the group consisting of: LRP1; LRP2; LRP3; LRP4; LRP5; LRP6; LRP8; or Porcupine.

In a yet further alternative method said RNAi molecule is derived from a nucleic acid molecule encoding an extracellular Wnt antagonist selected from the group consisting of: Dkk1; Dkk2; Dkk3; Dkk4; Frzb; or SARP1.

In a further preferred method of the invention said RNAi molecule is derived from a nucleic acid molecule encoding a Wnt cytoplasmic acting component selected from the group consisting of: APC; Axin1; Axin2; FRAT1; GSK3; ICAT; IDAX; Par 1; or TAB1.

Alternatively, said RNAi molecule is derived from a nucleic acid molecule encoding a Wnt nuclear acting component selected from the group consisting of: β-catenin; β-TRCP; CBP; CTBP1; HBP-1; Lef1; NLK; Pontin 52; or Reptin 52.

In a yet further preferred method of the invention said RNAi molecule is derived from a nucleic acid molecule which encodes a Wnt target gene selected from ASCL 1 or ASCL 2.

In a yet still further preferred method of the invention said RNAi molecule is derived from the group consisting of: FGF 5; msx 1; neurogenin 1; neurogenin 2; neurogenin 3 ; or PTEN.

According to a further aspect of the invention there is provided a method of treatment of an animal, preferably a human, comprising administering an effective amount of at least one RNAi molecule according to the invention, to a subject to be treated.

According to a yet further aspect of the invention there is provided a method of treatment of an animal, preferably a human, comprising administering an effective amount of at least one vector which includes an RNAi molecule according to the invention, to a subject to be treated.

An effective amount is an amount sufficient to induce the differentiation of at least one stem cell into at least one lineage restricted stem cell or differentiated stem cell.

According to a further aspect of the invention there is provided a lineage restricted stem cell or a differentiated stem cell obtainable by the method according to the invention.

In a preferred embodiment of the invention said lineage restricted stem cell is selected from the group consisting of: haemopoietic stem cell; neural stem cell; bone stem cell; muscle stem cell; mesenchymal stem cell; trophoblastic stem cell; epithelial stem cell (derived from organs such as the skin, gastrointestinal mucosa, kidney, bladder, mammary glands, uterus, prostate and endocrine glands such as the pituitary); endodermal stem cell (derived from organs such as the liver, pancreas, lung and blood vessels).

In a further preferred embodiment of the invention said cell is selected from the group consisting of: a nerve cell; a mesenchymal cell; a muscle cell (cardiomyocyte); a liver cell; a kidney cell; a blood cell (eg erythrocyte, CD4+ lymphocyte, CD8+ lymphocyte; panceatic β cell; epithelial cell (eg lung, gastric,); an endothelial cell.

According to a yet further aspect of the invention there is provided a cell culture comprising at least one lineage restricted stem cell or differentiated cell according to the invention.

According to a further aspect of the invention there is provided an organ comprising a lineage restricted stem cell or a differentiated stem cell according to the invention.

According to a yet further aspect of the invention there is provided a method of treatment of an animal, preferably a human, comprising administering a cell or organ according to the invention.

An embodiment of the invention will now be described by example only and with reference to the following figures and tables wherein:

Table 1 represents the nucleic acid sequences of Notch/Wnt target genes molecules from which RNAi molecules are derived;

Table 2 represents nucleic acid sequences of polycomb target genes from which RNAi molecules are derived;

Table 3 represents nucleic acid sequences of enhancers of trithorax and polycomb target genes from which RNAi molecules are derived;

Table 4 represents nucleic acid sequences of histone deacetylase target genes from which RNAi molecules are derived;

Table 5 represents nucleic acid sequences of histone acetylase target genes from which RNAi molecules are derived;

Table 6 represents nucleic acid sequences of ATP dependent chromatin modification target genes from which RNAi molecules are derived;

Table 7 represents a selection of antibodies used to monitor stem cell differentiation;

Table 8 represents nucleic acid probes used to assess mRNA markers of stem differentiation;

Table 9 represents protein markers of stem cell differentiation;

FIG. 1 illustrates stem cell differentiation is controlled by positive and negative regulators (A). The specific cell phenotypes that are derived are a direct result of positive and negative regulators which activate or suppress particular differentiation events. RNAi can be used to control both the initial differentiation of stem cells (A) and the ultimate fate of the differentiated cells D1 and D2 by repression of positive activators which would normally promote a particular cell fate;

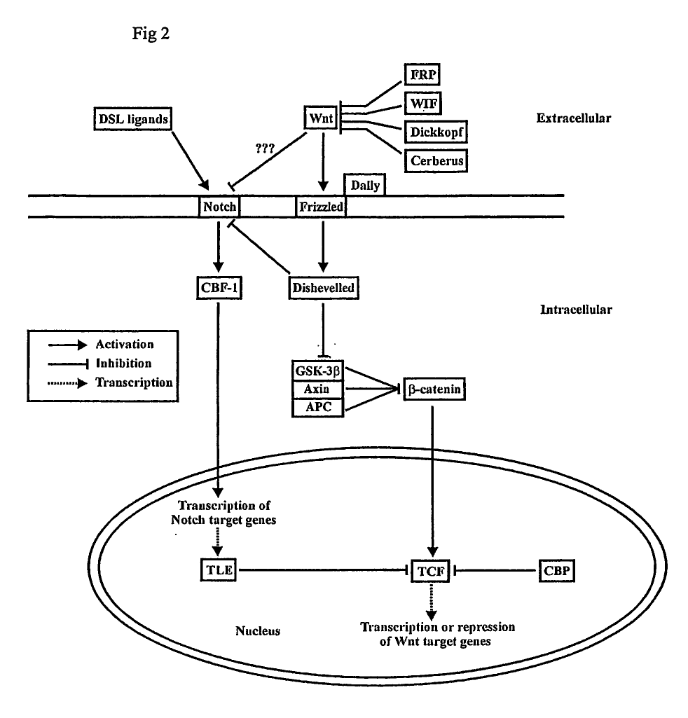

FIG. 2 represents (A) a schematic diagram illustrating the Notch and Wnt signalling pathways. The Notch and Wnt signaling pathways are shown.

### MATERIALS AND METHODS

Cell Culture

NTERA2 and 2102Ep human EC cell lines were maintained at high cell density as previously described (Andrews et al 1982, 1984b), in DMEM (high glucose formulation) (NMEM)(GIBCO BRL), supplemented with 10% v/v bovine foetal calf serum (GIBCO BRL), under a humidified atmosphere with 10% CO₂ in air.

Double Stranded RNA Synthesis

PCR primers were designed against the mRNA sequence of interest to give a product size of around 500bp. At the 5′ end of each primer was added a T7 RNA polymerase promoter, comprising one or other of the following sequences: TAATACGACTCACTATAGGG; AATTATAATACGACTCACTATA. PCR was performed using these primers on an appropriate cDNA source (e.g. derived from the cell type to be targeted) and the product cloned and sequenced to confirm its identity. Using the sequenced clone as a template, further PCRs were performed as required to generate template DNA for RNA synthesis. In each case, a quantity of the PCR was electrophoresed through agarose to verify product size and abundance, whilst the remainder was purified by alkaline phenol/chloroform extraction. RNA was synthesized using the Megascript kit (Ambion Inc.) according to the manufacturer's protocol and acid phenolchloroform extracted. The simultaneous synthesis of complementary strands of RNA in a single reaction circumvents the requirement for an annealing step. However, the quality and duplexing of the synthesized RNA was confirmed by agarose gel electrophoresis, with the desired products migrating as expected for double stranded DNA of the same length.

Treatment of Human Cells with dsRNA to Produce RNAi

The following method describes RNAi of cells cultured in 6 well plates. Volumes and cell numbers should be scaled appropriately for larger or smaller culture vessels. Cells were seeded at 500,000 per well on the day prior to treatment and grown in their normal medium. For each well to be treated, 9.5 μg of the double stranded RNA of interest was diluted in 300 μl of 150 mM NaCl. 21 μl of ExGen 500 (MBI Fermentas) was added to the diluted RNA solution and mixed by vortexing. The dsRNA/ExGen 500 mixture was incubated at room temperature for 10 minutes. 3 ml of fresh cell growth medium was then added, producing the RNAi treatment medium. Growth medium was aspirated from the culture vessel and replaced with 3 ml of RNAi treatment medium per well. Culture vessels were then centrifuged at 280 g for 5 minutes and returned to the incubator. After 12-18 hrs, RNAi treatment medium was replaced with normal growth medium and the cells maintained as required.

Total RNA Production

Growing cultures of cells were aspirated to remove the DME and foetal calf serum. Trace amounts of foetal calf serum was removed by washing in Phosphate-buffered saline. Fresh PBS was added to the cells and the cells were dislodged from the culture vessel using acid washed glass beads. The resulting cell suspension was centrifuged at 300×g. The pellets had the PBS aspirated from them. Tri reagent (Sigma, USA) was added at 1 ml per 10⁷ cells and allowed to stand for 10 mins at room temperature. The lysate from this reaction was centrifuged at 12000×g for 15 minutes at 4° C. The resulting aqueous phase was transferred to a fresh vessel and 0.5 ml of isopropanol/ml of trizol was added to precipitate the RNA. The RNA was pelleted by centrifugation at 12000×g for 10 mins at 4° C. The supernatant was removed and the pellet washed in 70% ethanol. The washed RNA was dissolved in DEPC treated double-distilled water.

Analysis of the Differentiation of EC Stem Cells Induced by Exposure to RNAi

Following exposure to RNAi corresponding to specific key regulatory genes, the subsequent differentiation of the EC cells was monitored in a variety of ways. One approach was to monitor the disappearance of typical markers of the stem cell phenotype; the other was to monitor the appearance of markers pertinent to the specific lineages induced. The relevant markers included surface antigens, mRNA species and specific proteins.

Analysis of Transfectants by Antibody Staining and FACS

Cells were treated with trypsin (0.25% v/v) for 5 mins to disaggregate the cells; they were washed and re-suspended to 2×10⁵ cells/ml. This cell suspension was incubated with 50 μl of primary antibody in a 96 well plate on a rotary shaker for 1 hour at 4° C. Supernatant from a myeloma cell line P3X63Ag8, was used as a negative control. The 96 well plate was centrifuged at 100 rpm for 3 minutes. The plate was washed 3 times with PBS containing 5% foetal calf serum to remove unbound antibody. Cell were then incubated with 50 μl of an appropriate FITC-conjugated secondary antibody at 4° C. for 1 hour. Cells were washed 3 times in PBS+5% foetal calf serum and analysed using an EPICS elite ESP flow cytometer (Coulter eletronics, U.K).(Andrews et. al., 1982)

Northern Blot Analysis of RNA

RNA separation relies on the generally the same principles as standard DNA but with some concessions to the tendency of RNA to hybridise with itself or other RNA molecules. Formaldehyde is used in the gel matrix to react with the amine groups of the RNA and form Schiff bases. Purified RNA is ran out using standard agarose gel electrophoresis. For most RNA a 1% agarose gel is sufficient. The agarose is made in 1× MOPS buffer and supplemeted with 0.66M formaldehyde.Dryed down RNA samples are reconstituted and denatured in RNA loading buffer and loaded into the gel. Gels are run out for apprx. 3 hrs (until the dye front is ¾ of the way down the gel).

The major problem with obtaining clean blotting using RNA is the presence of formaldehyde. The run out gel was soaked in distilled water for 20 mins with 4 changes, to remove the formaldehyde from the matrix. The transfer assembly was assembled in exactly the same fashion as for DNA (Southern) blotting.The transfer buffer used however was 10×SSPE. Gels were transfered overnight. The membrane was soaked in 2×SSPE to remove any agarose from the transfer assembly and the RNA was fixed to the memebrane. Fixation was acheived using short-wave (254 nM) UV light. The fixed membrane was baked for 1-2 hrs to drive off any residual formaldehyde.

Hybridisation was acheived in aqueous phase with formamide to lower the hybridisation temperatures for a given probe. RNA blots were prehybridised for 2-4 hrs in northern prehybridisation soloution. Labelled DNA probes were denatured at 95° C. for 5 mins and added to the blots. All hybridisation steps were carried out in rolling bottles in incubation ovens. Probes were hybridised overnight for at least 16 hrs in the prehybridisation soloution. A standard set of wash soloutions were used. Stringency of washing was acheived by the use of lower salt containing wash buffers. The following wash procedure is outlined as follows

| | | |
|---|---|---|
| 2X SSPE | 15 mins | room temp |
| 2X SSPE | 15 mins | room temp |
| 2X SSPE/0.1% SDS | 45 mins | 65° C. |
| 2X SSPE/0.1% SDS | 45 mins | 65° C. |
| 0.1X SSPE | 15 mins | room temp |

Preparation of Radiolabelled DNA Probes

The method of Feinberg and Vogelstein (Feinberg and Vogelstein, 1983) was used to radioactively label DNA. Briefly, the protocol uses random sequence hexanucleotides to prime DNA synthesis at numerous sites on a denatured DNA template using the Klenow DNA polymerase I fragment. Pre-formed kits were used to aid consistency. 5-100 ng DNA fragment (obtained from gel purifcation of PCR or restriction digests) was made up in water,denatured for 5 mins at 95° C. with the random hexamers. The mixture was quench cooled on ice and the following were added,
5 μl [α-32P] DATP 3000 Ci/mmol
1 μl of Klenow DNA polymerase (4U)

The reaction was then incubated at 37° C. for 1 hr. Unincorporated nucleotide were removed with spin columns (Nucleon Biosciences).

Production of cDNA

The enzymatic conversion of RNA into single stranded cDNA was achieved using the 3′ to 5′ polymerase activity of recombinant Moloney-Murine Leukemia Virus (M-MLV) reverse transcriptase primed with oligo (dT) and (dN) primers. For Reverse Transcription-Polymerase Chain Reaction, single. stranded cDNA was used. cDNA was synthesised from 1 μg poly (A)+RNA or total RNA was incubated with the following
1.0 μM oligo(dT) primer for total RNA or random hexcamers for mRNA
0.5 mM 10 mM dNIP mix
1 U/μl RNAse inhibitor (Promega)
1.0 U/μl M-MLV reverse transcriptase in manufacturers supplied buffer (Promega)

The reaction was incubated for 2-3 hours at 42° C.

Fluorescent Automated Sequencing

To check the specificity of the PCR primers used to generate the template used in RNAi production automatic sequencing was carried out using the prism fluorescently labelled chain terminator sequencing kit (erkin-Elmer) (Prober et al 1987). A suitable amount of template (200 ng plasmid, 100 ng PCR product), 10 μM sequencing primer (typically a 20mer with 50% G-C content) were added to 8 μl of prism pre-mix and the total reaction volume made up to 20 μl. 24 cycles of PCR (94° C. for 10 seconds, 50° C. for 10 seconds, 60° C. for 4 minutes). Following thermal cycling, products were precipitated by the addition of 2 μl of 3M sodium acetate and 50 μl of 100% ethanol. DNA was pelleted in an Eppendorf microcentrifuge at 13000 rpm, washed once in 70% ethanol and vacuum dried. Samples were analysed by the in-house sequencing Service (Krebs Institute). Dried down samples were resuspended in 4 μl of formamide loading buffer, denatured and loaded onto a ABI 373 automatic sequencer. Raw sequence was collected and analysed using the ABI prism software and the results were supplied in the form of analysed histogram traces.

Detection of Specific Protein Targets by SDS-PAGE and Western Blotting

To obtain cell lysates monolayers of cells were rinsed 3 times with ice-cold PBS supplemented with 2 mM CaCl₂. Cells were incubated with 1 ml/75 cm² flask lysis buffer (1% v/v NP40, 1% v/v DOC, 0.1 mM PMSF in PBS) for 15 min at 4° C. Cell lysates were transferred to eppendorf tubes and passed through a 21 gauge needle to shear the DNA. This was followed by freeze thawing and subsequent centrifugation (30 min, 4° C., 15000 g) to remove insoluble material. Protein concentrations of the supernatants were determined using a commercial protein assay (Biorad) and were adjusted to 1.3 mg/ml. Samples were prepared for SDS-PAGE by adding 4 times Laemmli electrophoresis sample buffer and boiling for 5 min. After electrophoresis with 16 μg of protein on a 10% polyacrylamide gel (Laemmli, 1970) the proteins were transferred to nitro-cellulose membrane with a pore size of 0.45 μm. The blots were washed with PBS and 0.05% Tween (PBS-T). Blocking of the blots occurred in 5% milk powder in PBS-T (60 min, at RT). Blots were incubated with the appropriate primary antibody. Horseradish peroxidase labelled secondary antibody was used to visualise antibody binding by ECL-(Amersham, Bucks., UK). Materials used for SDS-PAGE and western blotting were obtained from Biorad (California, USA) unless stated otherwise.

TABLE 7
Antibodies used to detect stem cell differentiation

| | | | Cell | Changes | |
|---|---|---|---|---|---|
| | | | phenotype | on Differ- | |
| Antibody | Class | Species | detected | entiation | Reference |
| TRA-1- | IgM | Mouse | Human EC, | ↓ | Andrews et. al., |
| 60 | | | ES cells. | differen- | 1984a |
| | | | | tiation | |
| TRA-1- | IgM | Mouse | Human EC, | ↓ | Andrews et. |
| 81 | | | ES cells. | differen- | al., 1984a |
| | | | | tiation | |
| SSEA3 | IgM | Rat | Human EC, | ↓ | Shevinsky et al |
| | | | ES cells. | differen- | 1982, Fenderson |
| | | | | tiation | et al 1987 |
| SSEA4 | IgG | Mouse | Human EC, | ↓ | Kannagi et al |
| | | | ES cells. | differen- | 1983 Fenderson |
| | | | | tiation | et al 1987 |
| A2B5 | IgM | Mouse | | ↑ | Fenderson et al |
| | | | | differen- | 1987 |
| | | | | tiation | |
| ME311 | IgG | Mouse | | ↑ | Fenderson et al |
| | | | | differen- | 1987 |
| | | | | tiation | |
| VIN-IS- | IgM | Mouse | | ↑ | Andrews et al |
| 56 | | | | differen- | 1990 |
| | | | | tiation | |
| VIN-IS- | IgG | Mouse | | ↑ | Andrews et al |
| 53 | | | | differen- | 1990 |
| | | | | tiation | |

TABLE 8
Probes used to assess mRNA markers of differentiation

| Gene | Cell Type |
|---|---|
| Synaptophysin | Neuron |
| NeuroD1 | Neuron |
| MyoD1 | Muscle |
| Collagens | Cartilage |
| Alpha-actin | Skeletal muscle |
| Smooth-muscle actin | Smooth muscle |

TABLE 9
Protein markers of differentiation, detected by Western Blot and/or
immunofluorescence.
The following antibodies were detected by the appropriate
commercially available antibodies

| Cell Type | Antigen |
|---|---|
| Neurons | Neurofilaments |
| Glial cells | GFAP |
| Epithelial cells | Cytokeratins |
| Mesenchymal cells | Vimentin |
| Muscle | Desmin |
| Muscle | Tissue specific actins |
| Connective tissue cells | Collagens |

### REFERENCES

Andrews P. W., Goodfellow P. N., Shevinsky L., Bronson D. L. and Knowles B. B. 1982. Cell surface antigens of a clonal human embryonal carcinoma cell line: Morphological and antigenic differentiation in culture. Int. J. Cancer. 29: 523-531.
Andrews P. W., Banting G. S., Damjanov I., Amaud D. and Avner P. 1984a Three monoclonal antibodies defining distinct differentiation antigens associated with different high molecular weight polypeptides on the surface of human embryonal carcinoma cells. Hybridoma. 3: 347-361.
Andrews P. W., Damjanov I., Simon D., Banting G., Carlin C., Dracopoli N. C. and Fogh J. 1984b. Pluripotent embryonal carcinoma clones derived from the human teratocarcinoma cell line Tera-2: Differentiation in vivo and in vitro. Lab. Invest. 50: 147-162.
Andrews P. W., Nudelman E., Hakomori S. -i. and Fenderson B. A. 1990. Different patterns of glycolipid antigens are expressed following differentiation of TERA-2 human embryonal carcinoma cells induced by retinoic acid, hexamethylene bisacetamide (HMBA) or bromodeoxyuridine (BUdR). Differentiation. 43: 131-138.
Fenderson B. A., Andrews P. W., Nudelman E., Clausen H. and Hakomori S.-i. 1987. Glycolipid core structure switching from globo- to lacto- and ganglio-series during retinoic acid-induced differentiation of TERA-2-derived human embryonal carcinoma cells. Dev. Biol. 122: 21-34.
Kannagi, R., Levery, S. B., Ishigami, F., Hakomori, S., Shevinsky, L. H., Knowles, B. B. and Solter, D. (1983) New globoseries glycosphingolipids in human teratocarcinoma reactive with the monoclonal antibody directed to a developmentally regulated antigen, stage-specific embryonic antigen 3. J. Biol. Chem. 258, 8934-8942.
Shevinsky, L. H., Knowles, B. B., Danjanov, I. and Solter, D. (1982) Monoclonal antibody to murine embryos defines a stage-specific embryonic antigen expressed on mouse embryos and human teratocarcinoma cells. Cell 30, 697-705.
Solter, D. and Knowles, B. B. (1978) Monoclonal antibody defining a stage-specific mouse embryonic antigen (SSEA-1). Proc. natl. Acad. Sci. USA 75, 5565-5569.
Recent progress in identifying genes regulating hematopoietic stem cell function and fate Craig T Jordan, Gary Van Zant Current Opinion in Cell Biology 1998, 10:716-720.
Singer M J, Selker E U. Genetic and epigenetic inactivation of repetitive sequences in Neurospora crassa: RIP, DNA methylation, and quelling. Curr Top Microbiol Immunol. 1995;197:165-77.
Matzke M A, Matzke A J. Gene silencing in plants: relevance for genome evolution and the acquisition of genomic methylation patterns. Novartis Found Symp. 1998;214:168-80; discussion 181-6. Review.
Stam M, de Brain R, van Blokland R, van der Hoorn R A, Mol J N, Kooter J M. Distinct features of post-transcriptional gene silencing by antisense transgenes in single copy and inverted T-DNA repeat loci. Plant J. 2000 January;21(1):27-42.
Montgomery M K, Xu S, Fire A. RNA as a target of double-stranded RNA-mediated genetic interference in Caenorhabditis elegans. Proc Natl Acad Sci U S A. 1998 Dec. 22;95(26): 15502-7.
Fire A, Xu S, Montgomery M K, Kostas S A, Driver S E, Mello C C. Potent and specific genetic interference by double-stranded RNA in Caenorhabditis elegans. Nature. 1998 February 19;391(6669):806-11
Kennerdell J R, Carthew R W. Heritable gene silencing in Drosophila using double-stranded RNA. Nat Biotechnol. 2000 August;18(8):896-898.
Shi H, Djikeng A, Mark T, Wirtz E, Tschudi C, Ullu E. Genetic interference in Trypanosoma brucei by heritable and inducible double-stranded RNA. RNA. 2000 July;6(7): 1069-76.
Wianny F, Zemicka-Goetz M. Specific interference with gene function by double-stranded RNA in early mouse development. Nat Cell Biol. 2000 February;2(2):70-5
Thomson J A, Itskovitz-Eldor J, Shapiro S S, Waknitz M A, Swiergiel J J, Marshall V S, Jones J M. Embryonic stem cell lines derived from human blastocysts. Science. 1998 Nov. 6;282(5391):1145-7.
Thomson J A, Kalishman J, Golos T G, Durning M, Harris C P, Becker R A, Hearn J P. Isolation of a primate embryonic stem cell line. Proc Natl Acad Sci U S A. 1995 Aug. 15;92(17):7844-8.
Prober J M, Trainor G L, Dam R J, Hobbs F W, Robertson C W, Zagursky R J, Cocuzza A J, Jensen M A, Baumeister K. A system for rapid DNA sequencing with fluorescent chain-terminating dideoxynucleotides. Science. 1987 Oct. 16;238(4825):336-41.
Feinberg A P, Vogelstein B. A technique for radiolabeling DNA restriction endonuclease fragments to high specific activity. Anal Biochem. 1983 Jul. 1;132(1):6-13.
Mullis K B, Faloona F A. Specific synthesis of DNA in vitro via a polymerase-catalyzed chain reaction. Methods Enzymol. 1987;155:335-50.
Scholer H R, Hatzopoulos A K, Balling R, Suzuld N, Gruss P. A family of octamer-specific proteins present during mouse embryogenesis: evidence for germline-specific expression of an Oct factor. EMBO J. 1989 September;8(9):2543-50.
Kraft H J, Mosselman S, Smits H A, Hohenstein P, Piek E, Chen Q, Artzt K, van Zoelen E J. Oct-4 regulates alternative platelet-derived growth factor alpha receptor gene promoter in human embryonal carcinoma cells. J Biol Chem. 1996 May 31;271(22):12873-8.
Reubinoff B E, Pera M F, Fong C Y, Trounson A, Bongso A Embryonic stem cell lines from human blastocysts: somatic differentiation in vitro. Nat Biotechnol. 2000 April; 18(4):399-404.
Shamblott M J, Axelman J. Wang S, Bugg E M, Littlefield J W, Donovan P J, Blumenthal P D, Huggins G R, Gearhart J D. Derivation of pluripotent stem cells from cultured human primordial germ cells. Proc Natl Acad Sci U S A. 1998 Nov. 10;95(23):13726-31.

Clarke D L, Johansson C B, Wilbertz J, Veress B, Nilsson E, Karlstrom H, Lendahl U, Frisen J. Generalized potential of adult neural stem cells. Science. 2000 Jun. 2;288(5471): 1660.

TABLE 1

Notch receptor processing factors:
DEFINITION AL527440 LTI NFL003_NBC3 Homo sapiens Nrarp 5
Prime, partial mRNA sequence.
ACCESSION AL527440 SEQ ID NO: 1
Gaggcgccttgggaccgcgtgggagccgcagccgaaccgagtagggaccgggaccgcgcggcgccgccgccccg
gccgggcccggcccccgcgagccgagcgcgcgcccccgtcgcccacccgggcgcggctggatgcggcggggtcccc
gcggcggcgacccccggccccgagcgcccggagcgcccagaggcggcgtgcggggcccggggacgccgcgcccts
tbgtgcgccgaggcgcgccccgagacagccgggggcccgcgccgcagccgccgcccgcgctgagccccggcccgg
cccgcggcccgcgcccggcggcagcntgagccaggccgagctgtccacctgctccgcgccgcagacgcagcgcatctt
ccaggaggctgtgcgcnagggcaacacgcaggagctgcagtggctgctgcagaacatgaccaactgcgagttcaacgtg
aactcgttcgggccgagggccagacggcgctgcaccagtcggtcatcgtcggcaacctggtgctcgtgaagctgctggt
caagttcggcgccgacatccgcctggccaaccgcgacggctggagcgcgctgcamatcgccgcgttcggtggccacca
ggacatcgtgctctatctcatcaccaaggcgaagtacgcggccagcgcsggtgtatgcccgccgggaccccggaccccg
gccctgcgcccgcgtcgtctctgctgtaccttcccgccaactacctcggtgcgcgcmcggctcgcaggccccgccagaa
ggcccgtggcaacggcgaatacggcgcgtgcgtcmcggccccagggtc
DEFINITION Homo sapiens E1A binding protein p300 (EP300), mRNA.
ACCESSION NM_001429 SEQ ID NO: 2
Ccttgtttgtgtgctaggctgggggggagagagggcgagagagagcgggcgagagtgggcaagcaggacgccgggct
gagtgctaactgcgggacgcagagagtgcggaggggagtcgggtcggagagaggcggcaggggccagaacagtggc
agggggcccggggcgcacgggctgaggcgacccccagccccctcccgtccgcacacacccccaccgcggtccagca
gccgggccggcgtcgacgctaggggggaccattacataacccgcgccccggccgtcttctcccgccgccgcggcgccc
gaactgagcccggggcgggcgctccagcactggccgccggcgtggggcgtagcagcggccgtattattatttcgcggaa
aggaaggcgaaggaggggagcgccggcgcgaggaggggccgcctgcgcccgccgccggagcggggcctcctcggt
gggctccgcgtcggcgcgggcgtgcgggcggcgctgctcggcccggccccctcggccctctggtccggccagctccgc
tcccggcgtccttgccgcgcctccgccggccgccgcgcgatgtgaggcggcggcgccagcctggctctcggctcgggc
gagttctctgcggccattaggggccggtgcggcggcggcgcggagcgcggcggcaggaggagggttcggagggtggg
ggcgcaggcccgggagggggcaccgggaggaggtgagctcttgtcgcctcctcctctccccccttttcgcccccgcc
tccttgtggcgatgagaaggaggaggacagcgccgaggaggaagaggttgatggcggcggcggagctccgagagacc
tcggctgggcaggggccggccgtggcgggccggggactgcgcctctagagccgcgagttctcgggaattcgccgcagc
ggaccggcctcggcgaatttgtgctcttgtgccctcctccgggcttgggccaggccggcccctcgcacttgcccttacctttt
ctatcgagtccgcatccctctccagccactgcgacccggcgaagagaaaaaggaacttcccccaccccctcgggtgccgt
cggagccccccagcccacccctgggtgcggcgcggggaccccgggccgaagaagagatttcctgaggattctggttttc
ctcgcttgtatctccgaaagaattaaaatggccgagaatgtggtggaccggggccgcctcagccaagcggcctaaact
ctcatctccggccctctcggcgtccgccagcgatggcacagattttggctctctatttgacuggagcacgacttaccagatg
aattaatcaactctacagaattgggactaaccaatggtggtgatattaatcagcttcagacaagtcttggcatggtacaagatg
cagcttctaaacataaacagctgtcagaattgctgcgatctggtagttcccctaacctcaatatgggagttggtggcccaggt
caagtcatggccagccaggcccaacagagcagtcctggattaggtttgataaatagcatggtcaaaagcccatgacacag
gcaggcttgacttctcccaacatggggatgggcactagtggaccaataagggtcctacgcagtcaacaggtatgatgaac
agtccagtaaatcagcctgccatgggaatgaacacagggacgaatgcgggcatgaatcctggaatgttggctgcaggcaa
tggacaagggataatgcctaatcaagtcatgaacggttcaattggagcaggccgagggcgacaggatatgcagtacccaa
acccaggcatgggaagtgctggcaacttactgactgagcctcttcagcagggctctccccagatgggaggacaaacagga
ttgagaggcccccagcctcttaagatgggaatgatgaacaaacccaatccttatggttcaccatatactcagaatcctggaca
gcagattggagccagtggccttggtctccagattcagacaaaaactgtactatcaaataacttatctccatttgctatggacaa
aaaggcagttcctggtggaggaatgcccaacatgggtcaacagccagccccgcaggtccagcagccaggtctggtgact
ccagttgcccaagggatgggttctggagcacatacagctgatccagagaagcgcaagctcatccagcagcagcttgttctc
cttttgcatgctcacaagtgccagcgccgggaacaggccaatggggaagtgaggcagtgcaaccttccacactgtcgcac
aatgaagaatgtcctaaaccacatgacacactgccagtcaggcaagtcttgccaagtggcacactgtgcatcttctcgacaa
atcatttcacactggaagaattgtacaagacatgattgtcctgtgtgtctccccctcaaaaatgctggtgataagagaaatcaa
cagccaattttgactggagcacccgttggacttggaaatcctagctctctaggggtgggtcaacagtctgcccccaacctaa
gcactgttagtcagattgatcccagctccatagaaagagcctatgcagctcttggactaccctatcaagtaaatcagatgccg
acacaaccccaggtgcaagcaaagaaccagcagaatcagcagcctgggcagtctccccaaggcatgcggcccatgagc
aacatgagtgctagtcctatgggagaaatggaggtgtaggagttcaaacgccgagtcttctttctgactcaatgttgcattca
gccataaattctcaaaaaccaatgatgagtgaaaatgccagtgtgccctccctgggtcctatgccaacagcagctcaaccat
ccactactggaattcggaaacagtggcacgaagatattactcaggatcttcgaaatcatcttgttcacaaactcgtccaagcc
atatttcctacgccggatcctgctgctttaaaagacagacggatggaaaacctagttgcatatgctcggaaagttgaagggg
acatgtatgaatctgcaaacaatcgagcggaatactaccaccttctagctgagaaaatctataagatccagaaagaactaga
agaaaaacgaaggaccagactacagaagcagaacatgctaccaaatgctgcaggcatggttccagtttccatgaatccag
ggcctaacatgggacagccgcaaccaggaatgacttctaatggccctctacctgacccaagtatgatccgtggcagtgtgc
caaaccagatgatgcatcgaataactccacaatctggtttgaatcaatttggccagatgagcatggcccagccccctattgta
ccccggcaaacccctcctcttcagcaccatggacagttggctcaacctggagctctcaacccgcctatgggctatgggcct
cgtatgcaacagccttccaaccagggccagttccttcctcagactcagttcccatcacagggaatgaatgtaacaaatatccc
tttggctccgtccagcggtcaagctccatgtctcaagcacaaatgtctagttcttcctgcccggtgaactctcctataatgcct
ccagggtctcaggggagccacattcactgtccccagcttcctcaaccagctcttcatcagaattcaccctcgcctgtacctag
tcgtacccccacccctcaccatactcccccaagcataggggctcagcagccaccagcaacaacaattccagcccctgttcc
tacaccaccagccatgccacctgggccacagtcccaggctctacatccccctccaaggcagacacctacaccaccaacaa
cacaacttccccaacaagtgcagccttcacttcctgctgcaccttctgctgaccagccccagcagcagcctcgctcacagca
gagcacagcagcgtctgttcctaccccaaacgcaccgctgcttcctccgcagcctgcaactccactttcccagccagctgta
agcattgaaggacaggtatcaatcctccatctactagtagcacagaagtgaattctcaggccattgctgagaagcagccttc
ccaggaagtgaagatggaggccaaaatggaagtggatcaaccagaaccagcagatacgcagccggaggatatttcaga
gtctaaagtggaagactgtaaaatggaatctaccgaaacagaagagagaagcactgagttaaaaactgaaataaaagagg
aggaagaccagccaagtacttcagctacccagtcatctccggctccaggacagtcaaagaaagattucaaaccagaag
aactacgacaggcactgatgccaacattggaggcactttaccgtcaggatccagaatcccttccctttcgtcaacctgtggac
cctcagcttttaggaatccctgattactttgatattgtgaagagccccatggatctttctaccattaagaggaagttagacactg
gacagtatcaggagccctggcagtatgtcgatgatatttggcttatgttcaataatgcctggttatataaccggaaaacatcac
gggtatacaaatactgctccaagctctctgaggtctttgaacaagaaattgacccagtgatgcaaagccttggatactgttgtg
gcagaatggagttctctccacagacactgtgctacggcaaacagttgtgcacaaataaactcgtgatgccacuattaca
gttaccagaacaggtatcatttctgtgagaagtgtttcaatgagatccaaggggagagcgtttctttgggggatgacccttccc
agcctcaaactacaataaataaagaacaattttccaagagaaaaaatgacacactggatcctgaactgtttgttgaatgtaca
gagtgcggaagaaagatgaatcagatctgtgtccttcaccatgagatcatctggcctgctggattcgtctgtgatggctgttta
aagaaaagtgcacgaactaggaaagaaaataagttttctgctaaaaggttgccatctaccagacttggcacctttctagagaa
tcgtgtgaatgactttctgaggcgacagaatcaccctgagtcaggagaggtaactgttagagtagttcatgcttctgacaaaa
ccgtggaagtaaaaccaggcatgaaagcaaggtttgtggacagtggagagatggcagaatcctttccataccgaaccaaa
gccctctttgcctttgaagaaattgatggtgttgacctgtgcttctttggcatgcatgttcaagagtatggctctgactgccctcc
acccaaccagaggagagtatacatatcttacctcgatagtgttcatttcttccgtcctaaatgcttgaggactgcagtctatcat
gaaatcctaattggatatttagaatatgtcaagaaattaggttacacaacagggcatatttgggcatgtccaccaagtgaggg
agatgattatatctccattgccatcctcctgaccagaagatacccaagcccaagcgactgcaggaatggtacaaaaaaatg
cttgacaaggctgtatcagagcgtatgtccatgactacaaggatatttttaaacaagctactgaagatagattaacaagtgca
aaggaattgccttatttcgagggtgatttctggcccaatgttctggaagaaagcattaaggaactggaacaggaggaagaag
agagaaaacgagaggaaaacaccagcaatgaaagcacagatgtgaccaagggagacagcaaaaatgctaaaaagaag
aataataagaaaaccagcaaaaataagagcagcctgagtaggggcaacaagaagaaacccgggatgcccaatgtatcta
acgacctctcacagaaactatatgccaccatggagaagcataaagaggtcttctttgtgatccgcctcattgctggccctgct
gccaactccctgcctcccattgttgatcctgatcctctcatcccctgcgatctgatggatggtcgggatgcgtttctcacgctgg
caagggacaagcacctggagttctcttcactccgaagagcccagtggtccaccatgtgcatgctggtggagctgcacacgc
agagccaggaccgctttgtctacacctgcaatgaatgcaagcaccatgtggagacacgctggcactgtactgtctgtgagg
attatgacttgtgtatcacctgctataacactaaaaaccatgaacacaaaatggagaaactaggccttggcttagatgatgaga
gcaacaaccagcaggctgcagccacccagagcccaggcgattctcgccgcctgagtatccagcgctgcatccagtctctg
gtccatgcttgccagtgtcggaatgccaattgctcactgccatcctgccagaagatgaagcgggttgtgcagcataccaagg
gttgcaaacggaaaaccaatggcgggtgccccatctgcaagcagctcattgccctctgctgctaccatgccaagcactgcc
aggagaacaaatgcccggtgccgttctgcctaaacatcaagcagaagctccggcagcaacagctgcagcaccgactaca
gcaggcccaaatgcttcgcaggaggatggccagcatgcagcggactggtgtggttgggcagcaacagggcctcccttcc
cccactcctgccactccaacgacaccaactggccaacagccaacaaccccgcagacgccccagcccacttctcagcctc
agcctacccctcccaatagcatgccaccctacttgcccaggactcaagctgctggccctgtgtcccagggtaaggcagcag
gccaggtgacccctccaacccctcctcagactgctcagccaccccttccagggcccccacctacagcagtggaaatggca
atgcagattcagagagcagcggagacgcagcgccagatggcccacgtgcaaatttttcaaaggccaatccaacaccagat
gcccccgatgactcccatggcccccatgggtatgaacccacctcccatgaccagaggtcccagtgggcatttggagccag
ggatgggaccgacagggatgcagcaacagccaccctggagccaaggaggattgcctcagccccagcaactacagtctg
ggatgccaaggccagccatgatgtcagtggcccagcatggtcaacctttgaacatggctccacaaccaggattgggccag
gtaggtatcagcccactcaaaccaggcactgtgtctcaacaagccttacaaaaccttttgcggactctcaggtctcccagctc
tcccctgcagcagcaacaggtgcttagtatccttcacgccaaaccccagctgttggctgcattcatcaagcagcgggctgcc
aagtatgccaactctaatccacaacccatccctgggcagcctggcatgccccaggggcagccagggctacagccacctac
catgccaggtcagcagggggtccactccaatccagccatgcagaacatgaatccaatgcaggcgggcgttcagagggct
ggcctgccccagcagcaaccacagcagcaactccagccacccatgggagggatgagcccccaggctcagcagatgaa
catgaaccacaacaccatgccttcacaattccgagacatcttgagacgacagcaaatgatgcaacagcagcagcaacagg
gagcagggccaggaataggccctggaatggccaaccataaccagttccagcaaccccaaggagttggctacccaccaca
gccgcagcagcggatgcagcatcacatgcaacagatgcaacaaggaaatatgggacagataggccagcttccccaggc
cttgggagcagaggcaggtgccagtctacaggcctatcagcagcgactccttcagcaacagatggggtcccctgttcagc
ccaaccccatgagcccccagcagcatatgctcccaaatcaggcccagtcaccacacctacaaggccagcagatccctaat
tctctctccaatcaagtgcgctctccccagcctgtcccttctccacggccaaagtcccagcccccacactccagtccttcccc
aaggatgcagcctcagccttctccacaccacgtttccccacagacaagttccccacatcctggactggtagctgcccaggc
caaccccatggaacaagggcattttgccagcccggaccagaattcaatgctttctcagcttgctagcaatccaggcatggca
aacctccatggtgcaagcgccacggacctgggactcagcaccgataactcagacttgaattcaaacctctcacagagtaca
ctagacatacactagagacaccttgtattttgggagcaaaaaaattattttctcttaacaagactttttgtactgaaaacaatttttt
tgaatctttcgtagcctaaaagaaaattttccttggaacacataagaactgtgaagtagccgtttgtggtttaaagcaaacatgc
aagatgaacctgagggatgatagaatacaaagaatatatttttgttatgggctggttaacaccagcctttcttcccctttgtgtgt
gtggttcaagtgtgcactgggaggaggctgaggcctgtgaagccaaacaatatgctcctgccttgcacctccaataggtttta
ttattttttttaaattaatgaacatatgtaatattaatgaacatatgtaatattaatagttattatttactggtgcagatggttgacatttt
tccctattttcctcactttatggaagagttaaaacatttctaaaccagaggacaaaaggggttaatgttactttgaaattacattct
atatatatataaatatatataaatatatattaaaataccagttttttttctctgggtgcaaagatgttcattcttttaaaaaatgtttaaa
aaaaa
DEFINITION Homo sapiens presenilin-associated protein mRNA, complete cds.
ACCESSION AF189289 SEQ ID NO: 3
Cggtgccgcggggatggcgggagccggagctggagccggagctcgcggcggagcggcggcgggggtcgaggctcg
agctcgcgatccaccgcccgcgcaccgcgcacatcctcgccaccctcggcctgcggctcagccctcggcccgcaggatg
gatggcgggtcagggggcctggggtctggggacaacgccccgaccaatgaggctcttttcgtggcactgggcgcgggc
gtgacggcgctcagccatcccctgctctacgtgaagctgctcatccaggtgggtcatgagccgatgccccccacccttggg
accaatgtgctggggaggaaggtcctctatctgccgagcttcttcacctacgccaagtacatcgtgcaagtggatggtaaga
tagggctgttccgaggcctgagtccacggctgatgtccaacgccctctctactgtgactcggggtagcatgaagaaggtttt
ccctccagatgagattgagcaggtttccaacaaggatgatatgaagacttccctgaagaaagttgtgaaggagacctcctac
gagatgatgatgcagtgtgtgtcccgcatgttggcccaccccctgcatgtcatctcaatgcgctgcatggtccagtttgtggg
acgggaggccaagtacagtggtgtgctgagctccattgggaagattttcaaagaggaagggctgctgggattcttcgttgga
ttaatccctcacctcctgggcgatgtggttttcttgtggggctgtaacctgctggcccacttcatcaatgcctacctggtggatg
acagcgtgagtgacaccccaggggggctgggaaacgaccagaatccaggttcccagttcagccaggccctggccatcc
ggagctataccaagttcgtgatggggattgcagtgagcatgctgacctaccccttcctgctagttggcgacctcatggctgtg
aacaactgcgggctgcaagctgggctccccccttaatccccagtgttcaaatcctggattcactgctggaagtacctgagtgt
gcagggccagctcttccgaggctccagcctgcttttccgccgggtgtcatcaggatcatgctttgccctggagtaacctgaat
catctaaaaaacacggtctcaacctggccaccgtgggtgaggcctgaccaccttgggacacctgcaagacgactccaacc
caacaacaaccagatgtgctccagcccagccgggcttcagttccatatttgccatgtgtctgtccagatgtggggttgagcg
ggggtggggctgcacccagtggattgggtcacccggcagacctagggaaggtgaggcgaggtggggagttggcagaat
ccccaacctcgcagatttgctgagtctgtcttgtgcagagggccagagaatggcttatgggggcccaggttggatgggga
aaggctaatggggtcagaccccaccccgtctacccctccagtcagcccagcgcccatcctgcagctcagctgggagcatc
attctcctgctttgtacatagggtgtggtcccctggcacgtggccaccatcatgtctaggcctatgctaggaggcaaatggcc
agctctgcctgtgtttttctcaacactacttttctgatatgagggcagcacctgcctctgaatgggaaatcatgcaactactcag
aatgtgtcctcctcatctaatgctcatctgtttaatggtgatgcctcgcgtacaggatctggttacctgtgcagttgtgaataccc
agaggttgggcagatcagtgtctctagtcctacccagttttaaagttcatggtaagatttgacctcatctcccgcaaataaatgt
attggtgatttggaaaaaaaaaaaaaaaaaaaa
DEFINITION Homo sapiens presenilin 1 (PSEN1) mRNA, complete cds.
ACCESSION A1F416717 SEQ ID NO: 4
Atgacagagttacctgcaccgttgtcctacttccagaatgcacagatgtctgaggacaaccacctgagcaatactgtacgta
gccagaatgacaatagagaacggcaggagcacaacgacagacggagccttggccaccctgagccattatctaatggacg
accccagggtaactcccggcaggtggtggagcaagatgaggaagaagatgaggagctgacattgaaatatggcgccaag
catgtgatcatgctcccctgtgactctctgcatggtggtggtcgtggctaccattaagtcagtcagcttttatacccggaa
ggatgggcagctaatctataccccattcacagaagataccgagactgtgggccagggagccctgcactcaattctgaatgc
tgccatcatgatcagtgtcattgttgtcatgactatcctcctggtggttctgtataaatacaggtgctataaggtcatccatgcct
ggcttattatatcatctctattgttgctgttctttttttcattcatttacttgggggaagtgtttaaaacctataacgttgctgtggacta
cattactgttgcactcctgatctggaattttggtgtggtgggaatgatttccattcactggaaaggtccacttcgactccagcag
gcatatctcattatgattagtgccctcatggccctggtgtttatcaagtacctccctgaatggactgcgtggctcatcttggctgt
gatttcggtatatgatttagtggctgttttgtgtccgaaaggtccacttcgtatgctggttgaaacagctcaggagagaaatgaa
acgctttttccagctctcatttactcctcaacaatggtgtggttggtgaatatggcagaaggagacccggaagctcaaaggag
agtatccaaaaattccaagtataatgcagaaagagcctgtctgcctcctgctgccatcaacctgctgtctatagctcccatggc
acccaggctgttcatgccaagggtgcctgcaggcccacggcacagaaagggagtcacaagacactgttgcagagaatg
atgatggcgggttcagtgaggaatgggaagcccagagggacagtcatctagggcctcatcgctctacacctgagtcacga
gctgctgtccaggaacttccagcagtatcctcgctggtgaagacccagaggaaaggggagtaaaacttggattgggagat
ttcattt
DEFINITION Homo sapiens presenilin 2 (Alzheimer disease 4) (PSEN2), transcript
variant 1, mRNA.
ACCESSION NM_000447 SEQ ID NO: 5
Cgagcggcggcggagcaggcatttccagcagtgaggagacagccagaagcaagctattggagctgaaggaacctgag
acagaagctagtcccccctctgaattttactgatgaagaaactgaggccacagagctaaagtgacttttcccaaggtcgccc
agcgaggacgtgggacttctcagacgtcaggagagtgatgtgagggagctgtgtgaccatagaaagtgacgtgttaaaaa
ccagcgctgccctctttgaaagccagggagcatcattcatttagcctgctgagaagaagaaaccaaaccgaaattca
acctctctgcggccccaagtgttcgtggtgcttccagaggcagggctatgctcacattcatggcctctgacagcgaggaaga
agtgtgtgatgagcggacgtccctaatgtcggccgagagccccacgcagcgctcctgccaggagggcaggcagggccc
agaggatggagagaacactgcccagtggagaagccaggagaacgaggaggacggtgaggaggaccctgacgctatg
tctgtagtggggttcccgggcggccgccaggcctggaggaagagctgaccctcaaatacggagcgaagcacgtgatcat
gctgtttgtgcctgtcactctgtgcatgatcgtggtggtagccaccatcaagtctgtgcgcttctacacagagaagaatggac
agctcatctacacgacattcactgaggacacaccctcggtgggccagcgcctcctcaactccgtgctgaacaccctcatcat
gatcagcgtcatcgtggttatgaccatcttcttggtggtgctctacaagtaccgctgctacaagttcatccatggctggttgatc
atgtctcactgatgctgctgttcctcttcacctatatctacctggggaagtgctcaagacctacaatgtggccatggactacc
ccaccctcttgctgctgtctggaacttcggggcagtgggcatggtgtgcatccactggaagggccctctggtgctgcagca
ggcctacctcatcatgatcagtgcgctcatggccctagtgttcatcaagtacctcccagagtggtccgcgtgggtcatcctgg
gcgccatctctgatgatctcgtggctgtgctgtgtcccaaagggcctctgagaatgctggtagaaactgcccaggagag
aaatgagcccatattccctgccctgatatactcatctgccatggtgtggacggttggcatggcgaagctggacccctcctctc
agggtgccctccagctcccctacgaxccggagatggaagaagactcctatgacagtagggggagccttcataccccga
DEFINITION Homo sapiens F-box protein SEL10 (SEL10) mRNA, complete cds.
ACCESSION AY008274 SEQ ID NO: 6
Ctcagcaggtcaggacatttggtaggggaaggttgaaagacaaaagcagcaggccttgggttctcagccttttaaaaacta
ttattaaatatatatttttaaaatttagtggttagagcttttagtaatgtgcctgtattacatgtagagagtattcgtcaaccaagag
gagttttaaaatgtcaaaaccgggaaaacctactctaaaccatggcttggttcctgttgatcttaaaagtgcaaaagagcctct
accacatcaaaccgtgatgaagatattagcattagcatcattgcccaaggcctccctttttgtcgaagacggatgaaagaa
agttggaccatggttctgaggtccgctctttttctttgggaaagaaaccatgcaaagtctcagaatatacaagtaccactgggc
ttgaccatgttcagcaacaccaacaacttttggggacctcagagcagccaatggccaagggcaacaacgacgccgaatta
catctgtccagccacctacaggcctccaggaatggctaaaaatgtttcagagctggagtggaccagagaaattgcttgcttta
gatgaactcattgatagttgtgaaccaacacaagtaaaacatatgatgcaagtgatagaaccccagtttcaacgagacttcatt
tcattgctccctaaagagttggcactctatgtgctttcattcctggaacccaaagacctgctacaagcagctcagacatgtcgc
tactggagaattttggctgaagacaaccttctctggagagagaaatgcaaagaagaggggattgatgaaccattgcacatca
agagaagaaaagtaataaaccaggtcatacacagtccatggaaaagtgcatacatcagacagcacagattgatacta
actggaggcgaggagaactcaaatctcctaaggtgctgaaaggacatgatgatcatgtgatcacatgcttacagttttgtggt
aaccgaatagttagtggttctgatgacaacactttaaaagtttggtcagcagtcacaggcaaatgtctgagaacattagtggg
acatacaggtggagtatggtcatcacaaatgagagacaacatcatcattagtggatctacagatcggacactcaagtgtgg
aatgcagagactggagaatgtatacacaccttatatgggcatacttccactgtgcgttgtatgcatcttcatgaaaaaagagtt
gttagcggttctcgagatgccactcttagggtttgggatattgagacaggccagtgtttacatgttttgatgggtcatgttgcag
cagtccgctgtgttcaatatgatggcaggagggttgttagtggagcatatgattttatggtaaaggtgtgggatccagagact
gaaacctgtctacacacgttgcaggggcatactaatagagtctattcattacagtttgatggtatccatgtggtgagtggatctc
ttgatacatcaatccgtgtttgggatgtggagacagggaattgcattcacacgttaacagggcaccagtcgttaacaagtgga
atggaactcaaagacaatattcttgtctctgggaatgcagattctacagttaaaatctgggatatcaaaacaggacagtgttta
caaacattgcaaggtcccaacaagcatcagagtgctgtgacctgtttacagttcaacaagaactttgtaattaccagctcagat
gatggaactgtaaaactatgggacttgaaaacgggtgaatttattcgaaacctagtcacattggagagtggggggagtggg
ggagttgtgtggcggatcagagcctcaaacacaaagctggtgtgtgcagttgggagtcggaatgggactgaagaaaccaa
gctgctggtgctggactttgatgtggacatgaagtgaagagcagaaaagatgaatttgtccaattgtgtagacgatatactcc
ctgcccttccccctgcaaaaagaaaaaaagaaaagaaaaagaaaaaaatcccngttctcagtggtgcaggatguggcttg
gggcaacagattgaaaagacctacagactaagaaggaaaagaagaagagatgacaaaccataactgacaagagaggcg
tctgctgtctcatcacataaaaggcttcactmgactgagggcagctttgcaatgagactttctaaatcaaaccaggtgcaa
ttatttctttattttcttctccagtggtcattggggcagtgttaatgctgaaacatcattacagattctgctagcctgttcttttaccac
tgacagctagacaccagaaaggaactgcaataatatcaaaacaagtactggttgactttctaattagagagcatctgcaaca
aaaagtcatttttctggagtggaaaagcttaaaaaaattactgtgaattgttittgtacagttatcatgaaaagcttttttttttattttt
tngccaaccattgccaatgtcaatcaatcacagtattagcctctgttaatctatuactgttgcttccatatacattcttcaatgcat
atgttgctcaaaggtggcaagttgtcctgggttctgtgagtcctgagatggattaattcttgatgctggtgctagaagtaggtct
tcaaatatgggattgttgtcccaaccctgtactgtactcccagtggccaaacttatttatgctgctaaatgaaagaaagaaaaa
agcaaattatttttttattttttttctgctgtgaagttttaggcccagactgaattccaaatttgctctagtttggttatggaaaaaaga
ctttttgccactgaaacttgagccatctgtgcctctaagaggctgagaatggaagagtttcagataataaagagtgaagtttgc
ctgcaagtaaagaattgagagtgtgtgcaaagcttattttcttttatctgggcaaaaattaaaacacattccttggaacagagct
attacttgcctgttctgtggagaaacttttctttttgagggctgtggtgaatggatgaacgtacatcgtaaaactgacaaaatattt
taaaaatatataaaacacaaaattaaaataaagttgctggtcagtcttagtgttttacagtatttgggaaaacaactgttacagttt
tattgctctgagtaactgacaaagcagaaactattcagtttttgtagtaaaggcgtcacatgcaaacaaacaaaatgaatgaaa
cagtcaaatggttgcctcattctccaagagccacaactcaagctgactgtgaaagtggtttaacactgtatcctaggcgatc
ttttttcctccttctgtttauutgttttatttatagtctgatttaaaacaatcagattcaagttggttaattttagttatgtaaca
acctgacatgatggaggaaaaaaacctttaaagggattgtgtctatggtttgattcacttagaaattttattttcttataacttaagt
gcaataaaatgtgttttttcatgtt
Target genes (transcription factors):
DEFINITION Homo sapiens HES-related repressor protein 1 HERP1 mRNA,
complete SEQ ID NO: 7
cds.
ACCESSION AF232238
Gtcgaccgcctgcccaggcccggggagggaggaggcgggcgtcagggtgctgcgccccgctcggcgtccgagcttcc
ggccgggctgtgccccgcgcggtcttcgccgggatgaagcgcccctgcgaggagacgacctccgagagcgacatggac
gagaccatcgacgtggggagcgagaacaattactcggggcaaagtactagctctgtgattagattgaattctccaacaaca
acatctcagattatggcaagaaagaaaaggagagggattatagagaaaaggcgtcgggatcggataaataacagtttatct
gagttgagaagacttgtgccaactgcttttgaaaaacaaggatctgcaaagttagaaaaagctgaaatattgcaaatgacagt
ggatcatttgaagatgcttcaggcaacagggggtaaaggctactttgacgcacacgctcttgccatggacttcatgagcata
ggattccgagagtgcctaacagaagttgcgcggtacctgagctccgtggaaggcctggactcctcggatccgctgcgggt
gcggcttgtgtctcatctcagcacttgcgccacccagcgggaggcggcggccatgacatcctccatggcccaccaccatc
atccgctccacccgcatcactgggccgccgccttccaccacctgcccgcagccctgctccagcccaacggcctccatgcc
tcagagtcaaccccttgtcgcctctccacaacttcagaagtgcctcctgcccacggctctgctctcctcacggccacgtttgc
ccatgcggattcagccctccgaatgccatccacgggcagcgtcgccccctgcgtgccacctctctccacctctctcttgtcc
ctctctgccaccgtccacgccgcagccgcagcagccaccgcggctgcacacagcttccctctgtccttcgcgggggcattc
cccatgcttcccccaaacgcagcagcagcagtggccgcggccacagccatcagcccgcccttgtcagtatcagccacgtc
cagtcctcagcagaccagcagtggaacaaacaataaaccttaccgaccctgggggacagaagttggagctttttaaatttttc
ttgaacttcttgcaatagtaactgaatgtcctccatttcagagtcagcttaaaacctctgcaccctgaaggtagccatacagatg
ccgacagatccacaaaggaacaataaagctatttgagacac
DEFINITION Homo sapiens HES-related repressor protein 2 HERP2 mRNA,
complete
cds.
ACCESSION AF232239 SEQ ID NO: 8
Tcagtgtgtgcggaacgcaagcagccgagagcggagaggcgccgctgtagttaactcctccctgcccgccgcgccgac
cctccccaggaacccccagggagccagcatgaagcgagctcaccccgagtacagctcctcggacagcgagctggacga
gaccatcgaggtggagaaggagagtgcggacgagaatggaaacttgagttcggctctaggttccatgtccccaactacatc
ttcccagattttggccagaaaaagacggagaggaataattgagaagcgccgacgagaccggatcaataacagtttgtctga
gctgagaaggctggtacccagtgcttttgagaagcagggatctgctaagctagaaaaagccgagatcctgcagatgaccgt
ggatcacctgaaaatgctgcatacggcaggagggaaaggttactttgacgcgcacgcccttgctatggactatcggagtttg
ggatttcgggaatgcctggcagaagttgcgcgttatctgagcatcattgaaggactagatgcctctgacccgcttcgagttcg
actggtttcgcatctcaacaactacgcttcccagcgggaagccgcgagcggcgcccacgcgggcctcggacacattccct
gggggaccgtcttcggacatcacccgcacatcgcgcacccgctgttgctgccccagaacggccacgggaacgcgggca
ccacggcctcacccacggaaccgcaccaccagggcaggctgggctcggcacatccggaggcgcctgctttgcgagcgc
cccctagcggcagcttcggaccggctccctgtggcacctccgcctccaaactgtcgctgcctctgctctcctcagtgc
ctccctgtcggccttccccttctctttcggctccttccacttactgtctcccaatgcactgagcccttcagcacccacgcaggct
gcaaaccttggcaagccctatagaccttgggggacggagatcggagctttttaaagaactgatgtagaatgagggagggg
aaagtttaaaatcccagctgggctggactgttgccaacatcaccttaaagtcgtcagtaaaagtaaaaaggaaaaaggtaca
aaaaactagtgttaaattttgttcaagacattaaattgaaatagtgagtataagccaacactttgtgataggtttgtactgtgcct
aatttactttgtaaaccagaatgattccgtttttgcctcaaaatttggggaatcttaacatttaggtatttttggtctgtttttctccttg
tatagttatggtctguagaattaattttccaaaccactatgcttgttaacatgattctgtttgttaatattttgacagattaag
gttgtataaataatattcttttggggggaggggaactatattgaattttatatttctgagcaaagcgttgacaaatcagatgatc
agcmatccaagaaagaagactagtaaattgtctgcctcctatagcagaaaggtgaatgtacaaaatgttggtggcctgaatc
catctgacagctgctggtatctgccaggactggcagttctgatttagttaggaggaccgctgataggttaggtctcatttgga
gtgttggtggaaaggaaactgaaggtaattgaatagaatacgcctgcatttaccagccccagcaacacaaagaatttttaatc
acacggatctcaaattcacaaatgttaacatggataagtgatcatggtgtgcgagtggtcaattgagtagtacagtggaaact
gttaaatgcataacctaattttcctgggactgccatattttcttaactggaaatttttatgtgagttttccttttggtgcatggact
gtggttgccaaggtatttaaaagggctttcctgcctccttctctttgatttatttaatttgatttgggctataaaatatcatttttcagg
tttattcttttagcaggtgtagttaaacgacctccactgaactgggtttgacctctgttgtactgatgtgttgtgactaaataaa
agaaagaacaaagtaaaaaaaaaaaaaaaaaaaaaaaaaaa
DEFINITION Homo sapiens hairy (Drosophila)-homolog (HRY), mRNA.
ACCESSION NM_005524 SEQ ID NO: 9
Atcacacaggatccggagctggtgctgataacagcggaatcccccgtctacctctctccttggtcctggaacagcgctact
gatcaccaagtagccacaaaatataataaaccctcagcacttgctcagtagttttgtgaaagtctcaagtaaaagagacacaa
acaaaaaattctttttcgtgaagaactccaaaaataaaattctctagagataaaaaaaaaaaaaaaaggaaaatgccagctga
tataatggagaaaaattcctcgtccccggtggctgctaccccagccagtgtcaacacgacaccggataaaccaaagacag
catctgagcacagaaagtcatcaaagcctattatggagaaaagacgaagagcaagaataaatgaaagtctgagccagctg
aaaacactgattttggatgctctgaagaaagatagctcgcggcattccaagctggagaaggcggacattctggaaatgaca
gtgaagcacctccggaacctgcagcgggcgcagatgacggctgcgctgagcacagacccaagtgtgctggggaagtac
cgagccggcttcagcgagtgcatgaacgaggtgacccgcttcctgtccacgtgcgagggcgttaataccgaggtgcgcac
tcggctgctcggccacctggccaactgcatgacccagatcaatgccatgacctaccccgggcagccgcaccccgccttgc
aggcgccgccaccgcccccaccgggacccggcggcccacagcacgcgccgttcgcgccgccgccgccatcgtgccc
atccccgggggcgcggcgccccctcccggcggcgccccctgcaagctgggcagccaggctggagaggcggctaaggt
gtttggaggcttccaggtggtaccggctcccgatggccagtttgctttcctcattcccaacggggccttcgcgcacagcggc
cctgtcatccccgtctacaccagcaacagcggcacctccgtgggccccaaagcagtgtcaccnccagcggcccctcgcu
acggcggactccatgtggaggccgtggcggaactgagggggctcaggccacccctcctcctaaactccccaacccacct
ctcttccctccggactctaaacaggaacttgaatactgggagagaagaggacttttttgattaagtggttactttgtgtttttttaat
ttctaagaagtactttttgtagagagagctgtattaagtgactgaccatgcactatatttgtatatattttatatgttcatattggatt
gcgcctttgtattataaaagctcagatgacatttcgtacacgagattcttatgtgatgccaagatgtttgaaatgct
cttaaaatatcttcctttggggaagtttatttgagaaaatataataaaagaaaaagtaaaggcaaaaaaaaaaaaaaaaaa
DEFINITION Homo sapiens hairy and enhancer of split (Drosophila) homolog 2
(HES2), mRNA. SEQ ID NO: 10
GenBank Ace: BG470458
Gcgcggggacactcgtgcgactggggcaaggtgcccacgggcttctctcctaatttgcctcacgtaacagttgagacccc
agagggcagcaaactgggtcgaattggagagccgtccaggcacagaaaaattcattcatctgcccagtgcccgctaact
gcggttccaggcgccggcgacgcaattcaagagctgacaccgcttccccgcggagcatggggctgcctcgccgggcag
gggacgcggcggagctgcgcaagagcctgaagccgctgctggagaagcgccggcgcgcgcgcatcaaccagagcct
gagccagcttaaggggctcatcctgccgctgctgggccgggaggatgcttctggctggcacacctggcttcccctccatgc
tcangaactgcttcctactctacatccaggctcctgagcagcccccagcttaagcttcagcagctccagggtaaatccatctc
cgcccagagctgactccggatctgccccagtgacaataacaagataaatgccttccattctcttcgtacttacaggatgtaatc
cacatatttacacagtgtttcatttgcggtaatgtttgtatagtttcttgttccttcgtacctatctgggaagcctgtg
DEFINITION Homo sapiens bHLH factor Hes4 (LOC57801), mRNA.
ACCESSION NM_021170 SE ID NO: 11
Atggccgcagacacgccggggaaaccgagcgcctcgccgatggcaggagcgccggccagcgccagccggacccca
gacaagccccggagcgcggccgagcaccgcaagtcctccaagccggtcatggagaagcggcgccgagcgcgtattaa
cgagagcctcgctcagctcaaaaccctcatcctggacgccctcagaaaagagagctcccgccactcgaagctggagaag
gcggacatcctggagatgaccgtgagacacctgcggagcctgcgtcgcgtgcaggtgacggccgcgctcagcgccgac
ccggccgttctgggcaagtaccgcgccggcttccacgagtgtctggcggaggtgaaccgcttcctggccggctgcgagg
gcgtcccggccgacgtgcgctcccgcctgctgggccacctggcagcctgcctgcgccagctgggaccctcccgccgcc
cggcctcgctgtccccggctgcccccgcagaggccccagcgcccgaggtctacgcgggccgcccgctgctgccatcgc
tcggcggccccttccctctgctcgcgccgccgctgctgccgggtctgacccgggcgctgcccgccgcccccagggcggg
gccgcagggcccgggtgggccctggaggccgtggctgcgctga
HESR1, SEQ ID NO:12
>gi | 5059322 | gb | af151522.1 | af151522 homo sapiens hairy and enhancer of split
related-1 (hesr-1) mrna, complete cds
cccaggaacccccagggagccagcatgaagcgagctcaccccgagtacagctcctcggacagcgagctgg
acgagaccatcgaggtggagaaggagagtgcggacgagaatggaaacttgagttcggctctaggttccat
gtccccaactacatcttcccagattttggccagaaaaagacggagaggaataattgagaagcgccgacga
gaccggatcaataacagtttgtctgagctgagaaggctggtacccagtgcttttgagaagcagggatctg
ctaagctagaaaaagccgagatcctgcagatgaccgtggatcacctgaaaatgctgcatacggcaggagg
gaaaggttactttgacgcgcacgcccttgctatggactatcggagtttgggatttcgggaatgcctggca
gaagttgcgcgttatctgagcatcattgaaggactagatgcctctgacccgcttcgagttcgactggttt
cgcatctcaacaactacgcttcccagcgggaagccgcgagcggcgcccacgcgggcctcggacacattcc
ctgggggaccgtcttcggacatcacccgcacatcgcgcacccgctgttgctgccccagaacggccacggg
aacgcgggcaccacggcctcacccacggaaccgcaccaccagggcaggctgggctcggcacatccggagg
cgcctgctttgcgagcgccccctagcggcagcctcggaccggtgctccctgtggtcacctccgcctccaa
actgtcgccgcctctgctctcctcagtggcctccctgtcggccttccccttctctttcggctccttccac
ttactgtctcccaatgcactgagcccttcagcacccacgcaggctgcaaaccttggcaagccctatagac
cttgggggacggagatcggagctttttaaagaactgatgtagaatgagggaggggaaagtttaaaatccc
agctgggctggactgttgccaacatcaccttaaagtcgtcagtaaaagtaaaaaggaaaaaggtacactttcagat
aattttttttttaaagactaaaggtttgttggtttacttttatcttttttaatgtttttttcat
catgtcatgtattagcagtttttaaaaactagttgttaaattttgttcaagacattaaattgaaatagtg
agtataagccaacactttgtgataggtttgtactgtgcctaatttactttgtaaaccagaatgattccgt
ttttgcctcaaaatttggggaatcttaacatttagtatttttggtctgtttttctccttgtatagttatg
gtctgtttttagaattaattttccaaaccactatgcttaatgttaacatgattctgtttgttaatatttt
gacagattaaggtgttgtataaataatattcttttggggggaggggaactatattgaattttatatttct
gagcaaagcgttgacaaatcagatgatcagctttatccaagaaagaagactagtaaattgtctgcctcct
atagcagaaaggtgaatgtacaaactgttggtggccctgaatccatctgaccagctgctggtatctgcca
ggactggcagttctgatttagttaggagagagccgctgataggttaggtctcatttggagtgttggtgga
aaggaaactgaaggtaattgaatagaatacgcctgcatttaccagccccagcaacacaaagaatttttaa
tcacacggatctcaaattcacaaatgttaacatggataagtgatcatggtgtgcgagtggtcaattgagt
agtacagtggaaactgttaaatgcataacctaattttcctgggactgccatattttcttttaactggaaa
tttttatgtgagttttccttttggtgcatggaactgtggttgccaaggtatttaaaagggctttcctgcc
tccttctctttgatttatttaatttgatttgggctataaaatatcatttttcaggtttattcttttagca
ggtgtagttaaacgacctccactgaactgggtttgacctctgttgtactgatgtgttgtgactaaataaa
aaa
HEY 1
SEQ ID NO:13
>gi | 20149602 | ref | nm_012258.2 | Homo sapiens hairy/enhancer-of-split related
with yrpw motif 1 (hey1), mrna
tcagtgtgtgcggaacgcaagcagccgagagcggagaggcgccgctgtagttaactcctccctgcccgcc
gcgccgaccctccccaggaacccccagggagccagcatgaagcgagctcaccccgagtacagctcctcgg
acagcgagctggacgagaccatcgaggtggagaaggagagtgcggacgagaatggaaacttgagttcggc
tctaggttccatgtccccaactacatcttcccagattttggccagaaaaagacggagaggaataattgag
aagcgccgacgagaccggatcaataacagtttgtctgagctgagaaggctggtacccagtgcttttgaga
aacagggatctgctaagctagaaaaagcagagatcctgcagatgaccgtggatcacctgaaaatgctgca
tacgscaggagggaaaggttactttgacgcgcacgcccttgctatggactatcggagtttgggatttcgg
gaatgcctggcagaagttgcgcgttatctgagcatcattgaaggactagatgcctctgacccgcttcgag
ttcgactggtttcgcatctcaacaactacgctccccagcgggaagccgcgagcggcgcccacgcgggcct
cggacacattccctgggggaccgtcttcggacatcacccgcacatcgcgcacccgctgttgctgccccag
aacggccacgggaacgcgggcaccacggcctcacccacggaaccgcaccaccagggcaggctgggctcgg
cacatccggaggcgcctgctttgcgagcgccccctagcggcagcttcggaccggtgctccctgtggtcac
ctccgcctccaaactgtcgctgcctctgctctcctcagtggcctccctgtcggccttccccttctctttc
ggctccttccacttactgtctcccaatgcactgagcccttcagcacccacgcaggctgcaaaccttggca
agccctatagaccttgggggacggagatcggagctttttaaagaactgacgtagaatgagggaggggaaa
gtttaaaatcccagctgggctggactgttgccaacatcaccttaaagtcgtcagtaaaagtaaaaaggaa
aaaggtacactttcagataattttttttttaaagactaaaggtttgttggtttacttttatcttttttaa
tgtttttttcatcatgtcatgtattagcagtttttaaaaactagttgttaaattttgttcaagacattaa
attgaaatagtgagtataagccaacactttgtgataggtttgtactgtgcctaatttactttgtaaacca
gaatgattccgtttttgcctcaaaatttggggaatcttaacatttaggtatttttggtctgtttttctcc
ttgtatagttatggtctgtttttagaattaattttccaaaccactatgcttaatgttaacatgattctgt
ttgttaatattttgacagattaaggtgttgtataaataatattcttttggggggaggggaactatattga
attttatatttctgagcaaagcgttgacaaatcagatgatcagctttatccaagaaagaagactagtaaa
ttgtctgcctcctatagcagaaaggtgaatgtacaaactgttggtggcctgaatccatctgaccagctgc
tggtatctgccaggactggcagttctgatttagttaggaggaccgccgataggttaggtctcatttggag
tgttggtggaaaggaaactgaaggtaattgaatagaatacgcctgcatttaccagccccagcaacacaaa
gaatttttaatcacacggatctcaaattcacaaatgttaacatggataagtgatcatggtgtgcgagtgg
tcaattgagtagtacagtggaaactgttaaatgcataacctaattttcccgggactgccatatttccttt
taactggaaatttttatgtgagttttccttttggtgcatggaactgtggttgccaaggtatttaaaaggg
ctttcctgcctccttctctttgatttatttaatttgatttgggctataaaatatcatttttcaggtttat
tcttttagcaggtgtagttaaacgacctccactgaactgggtttgacctctgttgtactgatgtgttgtg
actaaataaaaaagaaagaacaaagtaaaaaaaaaaaaaaaaaaaaaaaaaaa
HEY 2 SEQ ID NO: 14
>gi | 6912413 | ref | nm_012259.1 | Homo sapiens hairy/enhancer-of-split related
with yrpw motif 2 (hey2), mrna
tcggcgtccgagcttccggccgggctgtgccccgcgcggtcttcgccgggatgaagcgcccctgcgagga
gacgacctccgagagcgacatggacgagaccatcgacgtggggagcgagaacaattactcggggcaaagt
actagctctgtgattagattgaattctccaacaacaacatctcagattatggcaagaaagaaaaggagag
ggattatagagaaaaggcgtcgagatcggataaataacagtttatctgagttgagaagacttgtgccaac
tgcttttgaaaaacaaggatctgcaaagttagaaaaagctgaaatattgcaaatgacagtggatcatttg
aagatgcttcaggcaacagggggtaaaggctactttgacgcacacgctcttgccatggacttcatgagca
taggattccgagagtgcctaacagaagttgcgaggtacctgagctccgtggaaggcctggactcctcgga
tccgctgcgggtgcggcttgtgtctcatctcagcacttgcgccacccagcgggaggcggcggccatgaca
tcctccatggcccaccaccatcatccgctccacccgcatcactgggccgccgccttccaccacctgcccg
cagccctgctccagcccaacggcctccatgcctcagagtcaaccccttgtcgcctctccacaacttcaga
agtgcctcctgcccacggctctgctctcctcacggccacgtttgcccatgcggattcagccctccgaatg
ccatccacgggcagcgtcgccccctgcgtgccacctctctccacctctctcttgtccctccctgccaccg
tccacgccgcagccgcagcagccaccgcggctgcacacagcttccctctgtccttcgcgggggcattccc
catgcttcccccaaacgcagcagcagcagtggccgcggccacagccatcagcccgcccttgtcagtatca
gccacgtccagtcctcagcagaccagcagtggaacaaacaataaaccttaccgaccctgggggacagaag
ttggagctttctaaatttttcttgaacttcttgcaatagtaactgaatgtcctccatttcagagtcagct
taaaacctctgcaccctgaaggtagccatacagatgccgacagatccacaaaggaacaataaagctattt
gagacacaaacctcacgagtggaaatgtggtattctcttttttttctctcccttttttgtttggttcaag
gcagctcggtaactgacatcagcaacttttgaaaacttcacacttgttaccatttagaagtttactggaa
aatatatggaccgtaccatccagcagtgcatcagtatgtctgaattggggaagtaaaatgccctgactga
attctcttgagactagatgggacatacatatatagagagagagtgagagagtcgtgtttcgtaagtgcct
gagcttaggaagttttcttctggatatataacattgcacaagggaagacgagtgtggaggataggttaag
aaaggaaagggacagaagtcttgcaataggctgcagacattttaatacaatgccagagaagagtattctg
ctgaaaccaacaggttttactggtcaaaatgactgctgaaaataaaattcaagttgaaagatctagtttt
atcttagtttgccttctttgtacagacatgccaagaggtgacatttagcagtgaattggaataagcaatt
atttcatcagttctcagatcaacaagcatttctgctctgcctgcaggcccccaggcacttttttttttgg
atagctcaaaatatggtgctgctttatataaaccttacatttatatagtgaacatatgaggagttgccta
ccatgtgtccaccagaggctatttaattcatgccaacttgaaaactatacagtttgtaggagtttggttt
aatttattcagtttcattaggactatttttacatatttatcctcttcatcttatcctaatgatgcaacat
ctattcttgtcaccctttgggagaagttacatttctggaggtgatgaagcaaggagggagcactaggaag
agaaaagctacaatttttaaagctctttgtcaagttagtgattgcatttgatcccaaaacaagatgaatg
tatgcaatgggatgtacataagttatttttgcccatgcctaaactagtgctatgtaatggggttgtggtt
ttgtttttttagatttcgtttaatgacaaaataatctcttaatatgctgaaatcaagcacgtgagagttt
ttgtttaaaagataagagacacagcatgtattatgcacttcatttctctactgtgtggagaaagcaataa
acattatgagaatgttaaacgttatgcaaaattatacttttaaatatttgttttgaaattactgtaccta
gtcttttttgcattactttgtaacctttttctatgcaagagtctttacataccactaattaaatgaagtc
ctttttgactatt
HEYL SEQ ID NO:15
>gi | 19923414 | ref | nm_014571.2 | Homo sapiens hairy/enhancer-of-split related
with yrpw motif-like (hey1), mrna
accaggcagcctgcgttcgccatgaagcgacccaaggagccgagcggctccgacggggagtccgacggac
ccatcgacgtgggccaagagggccagctgagccagatggccaggccgctgtccacccccagctcttcgca
gatgcaagccaggaagaaacgcagagggatcatagagaaacggcgtcgagaccgcatcaacagtagcctt
tctgaattgcgacgcttggtccccactgcctttgagaaacagggctcttccaagctggagaaagccgagg
tcttgcagatgacggtggatcacttgaaaatgctccatgccactggtgggacaggattctttgatgcccg
agccctggcagttgacttccggagcattggttttcgggagtgcctcactgaggtcatcaggtacctgggg
gtccttgaagggcccagcagccgtgcagaccccgtccggattcgccttctctcccacctcaacagctacg
cagccgagatggagccttcgcccacgcccactggccctttggccttccctgcctggccctggtctttctt
ccatagctgtccagggctgccagccctgagcaaccagctcgccatcctgggaagagtgcccagccctgtc
ctccccggtgtctcctctcctgcttaccccatcccagccctccgaaccgctccccttcgcagagccacag
gcatcatcctgccagcccggaggaatgtgctgcccagtcgaggggcatcttccacccggagggcccgccc
cctagagaggccagcgacccctgtgcctgtcgcccccagcagcagggctgccaggagcagccacatcgct
cccctcctgcagtcttcctccccaacaccccctggtcctacagggtcggctgcttacgtggctgttccca
cccccaactcatcctccccagggccagctgggaggccagcgggagccatgctctaccactcctgggtctc
tgaaatcactgaaatcggggctttctgagctgccccttcaccaccccgccccaaggaataaggaaggttc
ttttaccaggagcccaaaaaagggcactgccttttctgctttgcttcgtggactggctcatatgtgaagg
cacgttctccagccatcagaggccccctcctcctccaacccatctctccttctcactgttatcccagctt
atccacccagctctcctggagctgttctggtctcagaggcttggttccatttctcacctgaacagatgag
tcctgggagagaccctcagagatccgcccagacccctctcctgccctctgcacaccagcagcaggcatga
accttgggtctgggaaaaagctttaacctgcagggcaccaggacccaaggcaggctgttccttggggcgg
tcagaccccagtcaggagcaatgactgactggctgcagccttcccacgccaagaggctggaacatagtgt
ctgcctcgcttcctggagatagtaactgagcaggggctacaaagaggtctcctgggaaccctgtctgccc
cttcccacctgtccttgggccacaccatcacactgaaccacaggacagaccctttctccaccacagccaa
ggcctggagactgggggcccagcagagcctgctcccaccctcctcccagcagcagacacccaccctctca
ctgactaacaggtccctgcacacagctggcctggtaaacccagctgggaggtttctaggcagcagcaaaa
ctctgtgacagggtgtcctcacaccaggccttggacagctctcccagacaggagccagggttgagcaatg
gagagcccagcccccacgtcttacagtcgccatcctccaggcgtgtggtccctccccattgggtgcacag
tgcagaggggccgtggccccatgtgatggtgcgcagagaggaacctcttgggattcagcaccagacgtct
gtgctgcctggtttgcatccggctcacagagcccagactgctggaacagccaaggactgtcaggctggac
aaaaataactgcaaggaggggcaagagaaaggatgattcgaggcaccttggcccttcaaggtcatgcagt
gggtcgagcgcctgagatcctgttcaccaggactccacagagctggctctgctcagaagccatttcattc
cccggctccaccctaggccactttttctaacagaggaaacaaatggtccagcagtcgttcccagcagaac
agcggagcctggactgacacccagtgggaccagtgttgccacaccagttgataaaatgcagaaaccctcc
tgtactcgttggtaaatatctactcccccaagtgaccccaggtgccccccaccgcctggcacttccccca
ggactcctacgatctggttactgcctggccgatccaaggctgtggagtcccagagccagcagttcactgg
tgctcattccacactggttagatacttcagttgtcacccctgggaagattctcccacctcctccctttga
tggaaccaccctccccagaggctgcattgaggagacaccacagactgaaaagtgagtttgcagaaacctt
ggggaaaagggccctttcaaagaagtggataagagggaggagatcattgagtgacccagaaagctctttt
gaaaagacagactcctcaaggagagataaagaggaaagcacctctttcattttttagtgtgagctaattc
catcagactgctgtcctcctggacccatctgagatgtgcagtagcaaggagaggggggatcattttagag
agtgggtcattggcagggagtgctccggagggaggcagaggggagactgtggtagaaggaagacagaact
cacacatgctcccaggattggggacagggacagaggaggtaacagaaggcaaaggccagtttccccgtta
tcatgaaggggcccactcaggacaggaacaaggacaactcctcctcctcctcctcctctcctgctgctcc
tgggataccaggtcagtgatgtagtcttgcagtttggcaacttcctagcctgagaatccctagtggggct
gtgggaaacacatttccacgttgcaagcatgcaactccaaagaatctgtgatgccactgaaatgagatgg
gaatgatccagctctttcagcatcttggttgaacttgctttcattgtccctgggatattgtggaaggaaa
ggtgactgtgtgatctgattctgtggtcaaggacttgcatcttgtgtttctatccccaagccttcctggt
gtctccaactcctaccccattgcatgggttgttgcggacatccaataaagatttttttagtgcttctgga
aaaaaaaaaaaaaaaaaaaaaaaaaaaaaaaaaaaaaaaaaaaaaaaaaaaaaacc
HRT1 SEQ ID NO:16
atgaagcgagctcaccccgagtacagctcctcggacagcgagctggacgagaccatcgaggtggagaagg
agagtgcggacgagaatggaaacttgagttcggctctaggttccatgtccccaactacatcttcccagat
tttggccagaaaaagacggagaggaataattgagaagcgccgacgagaccggatcaataacagtttgtct
gagctgagaaggctggtacccagtgcttttgagaagcagggatctgctaagctagaaaaagccgagatcc
tgcagatgaccgtggatcacctgaaaatgctgcatacggcaggagggaaaggttactttgacgcgcacgc
ccttgctatggactatcggagtttgggacttcgggaatgcctggcagaagtcgcgcgttatctgagcatc
attgaaggactagatgcctctgacccgcttcgagttcgactggtttcgcatctcaacaactacgcaacac
agcgggaagccgcgagcggcgcccacgcgggcctcggacacattccctgggggaccgtcttcggacatca
cccgcacatcgcgcacccgctgttgctgccccagaacggccacgggaacgcgggcaccacggcccaccca
acggaaccgcaccaccagggcaggctgggctcggcacatccggaggcgcctgctttgcgagcgcccccta
gcggcagcctcggaccggtgctccctgtggtcacctccgcctccaaactgtcgccgcctctgctcaacac
agtggcctccctgtcggccttccccttctctttcggctccttccacttactgtctcccaatgcactgagc
ccttcagcacccacgcaggctgcaaaccttggcaagccctatagaccttgggggacggagatcggagctt
tttaa
hrt2 SEQ ID NO:17
>gi | 1112795 | gb | af311884.1 | af311884 Homo sapiens hairy-related transcription
factor 2 mrna, complete cds
atgaagcgcccctgcgaggagacgacctccgagagcgacatggacgagaccatcgacgtggggagcgaga
acaattactcggggcaaagtactagctctgtgattagattgaattctccaacaacaacatctcagattat
ggcaagaaagaaaaggagagggattatagagaaaaggcgtcgggatcggataaataacagtttatctgag
ttgagaagacttgtgccaactgcttttgaaaaacaaggatctgcaaagttagaaaaaactgaaatattgc
aaatgacagtggatcatttgaagatgcttcaggcaacagggggtaaaggctactttgacgcacacgctct
tgccatggaattcatgagcataggattccgagagtgcctaacagaagttgcgcggtacctgagctccgtg
gaaggcctggactcctcggatccgctgcgggtgcggcttgtgtctcatctcagcacttgcgccacccagc
gggaggcggcggccatgacatcctccatggcccaccaccatcatccgctccacccgcatcactgggccgc
cgccttccaccacctgcccgcagccctgctccagcccaacggcctccatgcctcagagtcaaccccttgt
cgcctctccacaacttcagaagtgcctcctgcccacggctctgctctcctcaaggccacgtttgcccatg
cggattcagccctccgaatgccacccacgggcagcgtcgccccctgcgtgccacctctctccacctctct
cttgtccctctctgccaccgtccacgccgcagccgcagcagccaccgcggctgcacacagcttccctctg
tccttcgcgggggcattccccatgcttcccccaaacgcagcagcagcagtggccgcggccacagacatca
gcccgcccttgtcagtatcagccacgtccagtcctcagcagaccagcagtggaacaaacaataaacatta
ccgaccctgggggacagaagttggagctttttaa
hrt3
SEQ ID:NO: 18
>gi | 11127957 | gb | af311885.1 | aaf311885 Homo sapiens hairy-related transcription
factor 3 mrna, complete cds
atgaagcgacccaaggagccgagcggctccgacggggagtccgacggacccatcgacgtgggccaagagg
gccagctgagccagatggccaggccgctgtccacccccagctcttcgcagatgcaagccaggaagaaacg
cagagggatcatagagaaacggcgtcgagaccgcatcaacagtagcctttctgaattgcgacgcttggtc
cccactgcctttgagaaacagggctcttccaagctggagaaagccgaggtcttgcagatgacggtggatc
acttgaaaatgctccatgccactggtgggacaggattctttgatgcccgagccctggcagttgacttccg
gagcattggttttcgggagtgcctcactgaggtcatcaggtacctgggggtccttgaagggcccagcagc
cgtgcagaccccgtccggattcgccttctctcccacctcaacagctacgcagccgagatggagccttcgc
ccacgcccactggccctttggccttccctgcctggccctggtctttcttccatagctgtccagggctgcc
agccctgagcaaccagctcgccatcctgggaagagtgcccagccctgtcctccccggtgtctcctctcct
gcttaccccatcccagccctccgaaccgctccccttcgcagagccacaggcatcatcctgccagcccgga
ggaatgtgctgcccagtcgaggggcatcttccacccggagggcccgccccctagagaggccagcgacccc
tgtgcctgtcgcccccagcagcagggctgccaggagcagccacatcgctcccctcctgcagtcttcctcc
ccaacaccccctggtcctacagggtcggctgcttacgtggctgttcccacacccaactcatcctccccag
ggccagctgggaggccagcgggagccatgctctaccactcctgggtctctgaaatcactgaaatcggggc
tttctga
chf1 SEQ ID NO: 19
>gi | 6636408 | gb | af173901.1 | af173901 Homo sapiens basic helix-loop-helix
factor 1 (chf1) mrna, complete cds
cggccgcgtcgacgggaaagagccgctagagcagaccgcgccgccgccggagccgcgcctgcccaggcac
ggggagggaggaggcgggcgtcagggtgctgcgccccgctcggcgtccgagcttccggccgggctgtgcc
ccgcgcggtcttcgccgggatgaagcgcccctgcgaggagacgacctccgagagcgacatggacgagacc
atcgacgtggggagcgagaacaattactcggggcaaagtactagctctgtgattagattgaattctccaa
caacaacatctcagattatggcaagaaagaaaaggagagggattatagagaaaaggcgtcgggatcggat
aaataacagtttatctgagttgagaagacttgtgccaactgcttttgaaaaacaaggatctgcaaagtta
gaaaaagctgaaatattgcaaatgacagtggatcatttgaagatgcttcaggcaacagggggtaaaggat
actttgacgcacacgctcttgccatggacttcatgagcataggattccgagagtgcctaacagaagttgc
gcggtacctgagctccgtggaaggcctggactcctcggatccgctgcgggtgcggcttgtgtctcatctc
agcacttgcgccacccagcgggaggcggcggccatgacatcctccatggcccaccaccatcatccgctcc
acccgcatcactgggccgccgccttccaccacctgcccgcagccctgctccagcccaacggcctccatgc
ctcagagtcaaccccttgtcgcctctccacaacttcagaagtgcctcctgcccacggctctgctctcctc
acggccacgtttgccdatgcggattcagccctccgaatgccatccacgggcagcgtcgccccctgcgtgc
cacctctctccacctctctcttgtccctctctgccaccgtccacgccgcagccgcagcagccaccgcggc
tgcacacagcttccctctgtccttcgcgggggcattccccatgcttcccccaaacgcagcagcagcagtg
gccgcaggccacagccatcagcccgccctcgtcagtatcagccaatccagtcatcagcagaccagcagtg
gaacaaacaataaaccttaccgaccctgggggacagaagttggagctttttaaatttttcttgaacttct
tgcaatagtaactgaatgtcctccatttcagagtcagcttaaaacctctgcaccctgaaggtagccatac
agatgccgacagatccacaaaggaacaataaagctatttgagacacaaaaagtcgacgcggccgcgaatt
c
chf2 SEQ ID NO:20
>gi | 5802652 | af176422.1 | af176422 Homo sapiens cardiovascular helix-loop-
helix factor 2 (chf2) mrna, complete cds
gaattcgcggccgcgtcgaccgagagcggagaggcgccgctgtagttaactcctccctgccgcccgcgcc
gaccctccccaggaacccccagggagccagcatgaagcgagctcaccccgagtacagatcctcggaaagc
gagctggacgagaccatcgaggtggagaaggagagagcggacgagaatggaaacttgagttcggctctag
gttccatgtccccaactacatcttcccagattttggccagaaaaagacggagaggaataattgagaagcg
ccgacgagaccggatcaataacagtttgtctgagctgagaaggctggtacccagtgcttttgagaagaag
ggatctgctaagctagaaaaagccgagatcctgcagatgaccgtggatcacctyaaaatgctgcatacgg
caggagggaaaggttactttgacgcgcacgcccttgctatggactatcggagtttgggatttcgggaatg
cctggcagaagttgcgcgttatctgagcatcattgaaggactagatgcctctgacccgcttcgagttcga
ctggttccgcatcccaacaactacgcttcccagcgggaagccgcgagcggcgcacaagcgggcctaggag
acattccctgggggaccgtcttcggacatcacccgcacatcgcgcacccgctgttgctgcaacagaaagg
ccacgggaacgcgggcaccacggcctcacccacggaaccgcaccaccagggcaggctgggctcggcacat
ccggaggcgcctgctttgcgagcgccccctagcggcagcctcggaccggtgctccctgtggtcacctccg
cctccaaactgtcgccgcctctgctctcctcagtggcctccctgtcggccttccccttctctttcggctc
cttccacttactgtctcccaatgcactgagcccttcagcacccacgcaggctgcaaaccttggcaagccc
tatagaccttgggggacggagatcggagcttttaaaagaactgatgtagaatgagggaggggaaagtaaa
aaatccagctgggctggactgttgccaacatcaccttaaagtcgtcagtaaaagtaaaaaggaaaaagga
tttcatcatgtcatgtattagcagtttttaaaaaactagttgttaaattttgttcaagacattaaaaaga
aatagtgagtataagccaacactttgtgataggtttgtactgtgcctaatttactttgtaaaccagaatg
atattttgacagattaaggtgttgtataaataatattcttttggggggaggggaactatattgaatttta
tatttctgagcaaagcgttgacaaatcagatgatcagctttatccaagaaagaagactagtaaaatgaat
gcctcctatagcagaaaggtgaatgtacaaactgttggtggccctgaatccatctgaccagctgctggta
tctgccaggactggcagttctgatttagttaggagagagccgctgataggttaggtctcatttggagtgt
tggtggaaaggaaactgaaggtaattgaatagaatacgcctgcatttaccagccccagcaacacaaagaa
tttttaatcacacggatctcaaattcacaaatgttaacatggataagtgatcatggtgtgcgagtggtca
attgagtagtacagtggaacctgttaaatgcataacctaattttcctgggactgccatattttcttttaa
ctggaaatttttatgtgagttttccttttggtgcatggaactgtggttgccaaggtatttaaaagggctt
tcctgcctccttctctttgatttatttaatttgatttgggctataaaatatcatttttcaggtttattct
tttagcaggtgtagttaaacgacctccactgaactgggtttgacctctgttgtactgatgtgttgtgaat
aaataaaaaagaaagaacaaagaaaaaaaaaaaaaaaaaaaaaaaaaaaaaaaaaaaaaaaaaaa
gridlock SEQ ID NO: 21
>gi | 7274451 | gb | af237949.1 | af237549 homo sapiens gridlock mrna, complete cds
agggtgctgcgccccgctcggcgtccgagcttccggccgggctgtgccccgcgcggtcttcgccgggatg
aagcgcccctgcgaggagacgacctccgagagcgacatggacgagaccatcgacgtggggagcgagagca
attactcggggcaaagtactagctctgtgattagattgaattctccaacaacaacatctcagataatggc
aagaaagaaaaggagagggattatagagaaaaggcgtcgggatcggataaataacagtttatctgagttg
agaagacttgtgccaactgcttttgaaaaacaaggatctgcaaagttagaaaaagatgaaatattgcaaa
tgacagtggatcatttgaagatgcttcaggcaacagggggtaaaggctactttgacgcacacgctcttgc
catggacttcatgagcataggattccgagagtgcctaacagaagttgcgcggtacctgagctccgtggaa
ggcctggactcctcggatccgctgcgggtgcggcttgtgtctcatctcagcacttgcgccacccagcggg
aggcggcggccatgacatcctccatggcccaccaccatcatccgctccacccgcatcactgggccgccgc
cttccaccacctgcccgcagccctgctccagcccaacggcctccatgcctcagagtcaaccccttgtcgc
ctctccacaacttcagaagtgcctcctgcccacggctctgctctcctcacggccacgtttgcccatgcgg
attcagccctccgaatgccatccacgggcagcgtcgccccctgcgtgccacctctctccacctctctctt
gtccctctctgccaccgtccacgccgcagccgcagcagccaccgcggctgcacacagcttccctctgtcc
ttcgcgggggcattccccatgcttcccccaaacgcagcagcagcagtggccgcggccacagccatcagcc
cgcccttgtcagtatcagccacgtccagtcctcagcagaccagcagtggaacaaacaataaaccttaccg
accctgggggacagaagttggagctttttaaatttttcttgaacttcttgcaatagtaactgaatgtcct
ccatttcagagtcagcttaaaacctctgcaccctgaaggtagccatacagatgccgacagatccacaaag
gaacaataaagctatttgagacacaaacctcacgagtggaaatgtggtattctcttttttttctctccct
ttagaagtttcctggaaaatatatggaccgtaccatccagcagtgcatcagtaagacagaattggggaag
taaaatgccctgactgaattctcttgagactagatgggacatacatatatagagagagagtgagagagtc
gtgtttcgtaagtgcctgagcttaggaagttttcttctggatatataacattgcacaagggaagacgagt
gtggaggataggttaagaaaggaaagggacagaagtcttgcaataaccgcagaca
DEFINITION Homo sapiens hairy/enhancer of split 6 (HES6) mRNA, complete cds.
ACCESSION AF260237 SEQ ID NO:22
Ctccggtccccgccgctccccgtccccgctgctcctagcccctgccgcgtccccggcggagcgggcatggcgccaccc
gcggcgcctggccgggaacgtgtgggccgtgaggatgaggacggctgggagacgcgaggggaccgcaaggcccgga
agcccctggtggagaagaagcggcgcgcgcggatcaacgagagcctgcaggagctgcggctgctgctggcgggcgcc
gaggccaagctggagaacgccgaagtgctggagctgacggtgcggcgggtccagggtgtgctgcggggccgggcgcg
cgagcgcgagcagctgcaggcggaagcgagcgagcgcttcgctgccggctacatccagtgcatgcacgaggtgcacac
gttcgtgtccacgtgccaggccatcgacgctaccgtcgctgccgagctcctgaaccatctgctcgagtccatgccgctgcgt
gagggcagcagcttccaggatctgctgggggacgccctggcggggccacctagagcccctggacggagtggctggcct
gcggggggcgctccgggatccccaatacccagccccccgggtcctggggacgacctgtgctccgacctggaggaggcc
cctgaggctgaactgagtcaggctcctgctgaggggcccgacttggtgcccgcagccctgggcagcctgaccacagccc
aaattgcccggagtgtctggaggccttggtgaccaatgccagccagagtcctgcgggggtgggcccggccctccctggat
ctcctccctcctcccaggggttcagatgtggtggggtagggccctggaagtctcccaggtcttccatccctcctctgatggat
ggcttgcagggaagcccctggtaaccagcccagtcaggccccagccccgtttcttaagaaacttttagggaccctgcagct
ctggagtgggtggagggagggagctacgggcaggaggaagaattttgtagagctgccagcgctctcccaggttcaccca
cccaggcttcaccagccctgtgcgggctctgggggcagaggtggcagaaatggtgctgggcactagtgttccaggcagc
cctgggctaaacaaaagcttgaacttgccacttcagcggggagatgagaggcaggtgcactgagctgcactgcccagagc
tgtgatgctctgtacatcttgtttgtagcacacttgagtttgtgtattccattgacatcaaatgtgacaattttactaaataaagaatt
ttggagttagtt
DEFINITION Homo sapiens bHLH factor Hes7 (HES7), mRNA.
ACCESSION NM_032580 SEQ ID NO: 23
Atggtcacccgggatcgagctgagaatagggacggccccaagatgctcaagccgcttgtggagaagcggcgccggga
ccgcatcaaccgcagcctggaagagctgaggctgctgctgctggagcggacccgggaccagaacctccggaacccgaa
gctggagaaagcggagatattggagttcgccgtgggctacttgagggagcgaagccgggtggagcccccgggggttccc
cggtccccagtccaggacgccaaggcgctcgccagctgctacttgtccggtttccgcgagtgcctgcttcgcttggcggcc
atcgcgcacgacgccagcccgaccgcccgcgcccagctcttctccgcgctgcacggctatctgcgccccaaaccgcccc
ggcccaagccggtagatccgaggcctccagcgccgcgcccatccctggaccccgccgcaccggcccttggccctgcgc
tgcaccagcgccccccagtgcaccagggccaccctagcccgcgctgcgcatggtccccatccctctgctccccgcgcgc
cggggattctggcgcgccggcgcccctcaccggactgctgccgccgccaccgccgcctcacagacaagacggggcgc
ccaaggccccgctgcccccgccgcccgctttctggagaccttggccctga
Wnt pathway
Wnt ligand processing factors:
DEFINITION Homo sapiens low density lipoprotein-related protein 1
(alpha-2-macroglobulin receptor) (LRP1), mRNA.
ACCESSION XM_017228 SEQ ID NO:24
Cagcggtgcgagctccaggcccatgcactgaggaggcggaaacaaggggagcccccagagctccatcaagccccctc
caaaggctcccctacccggtccaagccccccacccaccctccccgcctcctcccaattgtgcatttttgcagccggaggcg
gctccgagatggggctgtgagcccgcccggggagggggaaagagcagcgaggagtgaagcgggggggtggggtga
agggtttggatttcggggcagggggcgcacccccgtcagcaggccctccccaaggggctcggaactctacctcttcaccc
acgcccctggtgcgctttgccgaaggaaagaataagaacagagaaggaggagggggaaaggaggaaaagggggacc
ccccaactggggggggtgaaggagagaagtagcaggaccagaggggaaggggctgctgcttgcatcagcccacaccat
gctgaccccgccgttgctcctgctgctgcccctgctctcagctctggtcgcggcggctatcgacgcccctaagacttgcagc
cccaagcagtttgcctgcagagatcaataacctgtatctcaaagggctggcggtgcgacggtgagagggactgcccaga
cggatctgacgaggcccctgagatttgtccacagagtaaggcccagcgatgccagccaaacgagcatactgcctgggta
ctgagctgtgtgttcccatgtcccgcctctgcaatggggtccaggactgcatggacggctcagatgaggggccccactgcc
gagagctccaaggcaactgctctcgcctgggctgccagcaccattgtgtccccacactcgatgggaccacctgctactgca
acagcagctttcagcttcaggcagatggcaagacctgcaaagattttgatgagtgctcagtgtacggcacctgcagccagct
atgcaccaacacagacggctccttcatatgtggctgtgttgaaggatacctcctgcagccggataaccgctcctgcaaggcc
aagaacgagccagtagaccggccccctgtgctgttgatagccaactcccagaacatcttggacacgtacctgagtggggc
ccaggtgtctaccatcacacctacgagcacgcggcagaccacagccatggacttcagctatgccaacgagaccgtatgct
gggtgcatgttggggacagtgctgctcagacgcagctcaagtgtgcccgcatgcctggcctaaagggcttcgtggatgag
cacaccatcaacatctccctcagtctgcacc
DEFINITION Homo sapiens low density lipoprotein-related protein 2 (LRP2),
mRNA.
ACCESSION XM_002645 SEQ ID NO:25
Gcagacctaaggagcgttcgctagcagaggagctgccggtgcggtgtgctacgcgcgcccacctcccggggaaggaa
cggcgaggccggggaccgtcgcggagatggatcgcgggccggcagcagtggcgtgcacgctgctcctggctctcgtcg
cctgactagcgccggccagtggccaagaatgtgacagtgcgcattttcgctgtggaagtgggcattgcatccctgcagact
ggaggtgtgatgggaccaaagactgttcagatgacgcggatgaaattggctgcgctgttgtgacctgccagcagggctattt
caagtgccagagtgagggacaatgcatccccaactcctgggtgtgtgaccaagatcaagactgtgatgatggctcagatga
acgtcaagattgctcacaaagtacatgctcaagtcatcagataacatgctccaatggtcagtgtatcccaagtgaatacaggt
gcgaLacacgtcagagactgccccgatggagctgatgagaatgactgccagtacccaacatgtgagcagcttacttgtgaca
atggggcctgctataacaccagtcagaagtgtgattggaaagttgattgcagggactcctcagatgaaatcaactgcagctg
agatatgcttgcacaatgagttttcatgtggcaatggagagtgtatccctcgtgcttatgtctgtgaccatgacaatgattgcca
agacggcagtgacgaacatgcttgcactatccgacctgcggtggttaccagttcacttgccccagtggccgatgcatttatc
aaaactgggtttgtgatggagaagatgactgtaaagataatggagatgaagatggatgtgaaagcggtcctcatgatgttcat
aaatgttccccaagagaatggtcttgcccagagtcgggacgatgcatctccatttataaagtttgtgatgggattttagattgcc
caggaagagaagatgaaaacaacactagtaccggaaatactgtagtatgactctgtgctctgccttgaactgccagtacca
gtgccatgagacgccgtatggaggagcgtgtttttgtcccccaggttatatcatcaaccacaatgacagccgtacctgtgttg
agttgatgattgccagatatggggaatttgtgaccagaagtgtgaaagccgacctggccgtcacctgtgccactgtgaaga
agggtatatcttggagcgtggacagtattgcaaagctaatgattcctttggcgaggcctccattatcttctccaatggtcgggat
ttgttaattggtgatattcatggaaggagcttccggatcctagtggagtctcagaatcgtggagtggccgtgggtgtggctttc
cactatcacctgcaaagagttttttggacagacaccgtgcaaaataaggttttttcagttgacattaatggtttaaatatccaaga
ggttctcaatgmctgttgaaaccccagagaaactggctgtggactgggttaataataaaatctatctagtggaccaaggt
caaccgcatagatatggtaaatttggatggaagctatcgggttacccttataactgaaaacttggggcatcctagaggaattg
ccgtggacccaactgttgttatttatttttctcagattgggagagcctttctggggaacctaagctggaaagggcattcatgg
atggcagcaaccgtaaagacttggtgaaaacaaagctgggatggcctgctggggtaactctggatatgatatcgaagcgtg
tttactgggttgactctcggtttgattacattgaaactgtaacttatgatggaattcaaagggaagactgtagttcatggaggctc
cctcattcctcatccctttggagtaagcttatttgaaggcagtgttctttacagattggacaaagatggccgtgctgaaggca
aacaagttcacagagaccaacccacaagtgtactaccaggcttccctgaggccctatggagtgactgtttaccattccctca
gacagccctatgctaccaatccgtaaagataacaatgggggctgtgagcaggtctgtgtcctcagccacagaacagata
atgatggtttgggtttccgtgcaagtgcacattcggcttccaactggatacagatgagcgccactgcattgctgttcagaattt
catcattttttcatcccaagUgctattcgtgggatcccgttcaccttgtctacccaggagatgtcatggttccagtttcgggga
atccttctttctttgtcgggattgattttgacgcccaggacagcactatctttttttcagatatgtcaaaacacatgatttttaagca
aaagattgatggcacaggaagagaaattctcgcagctaacagggtggaaaatgttgaaagtttggcttttgattggatttcaa
agaatctctattggacagactctcattacaagagtatcagtgtcatgaggctagctgataaaacgagacgcacagtagttcag
tatttaaataacccacggtcggtggtagttcatccttttgccgggtatctattcttcactgattggttccgtcctgctaaaattatga
gagcatggagtgacggatctcacctcttgcctgtaataaacactactcttggatggcccatggcttggccatcgattgggct
gcttcacgattgtactgggtagatgcctattttgataaaattgagcaaagcaccagatgaagacagaagaagactgggc
catatagagcagatgacacatccgtttggacttgccatctttggagagcatttattttttactgactggagactgggtgccattat
tcgagtcaggaaagcagatggtggagaaatgaaagttatccgaagtggcattgcttacatactgcatttgaaatcgtatgatg
tcaacatccagactggttctaacgcctgtaatcaacccacgcatcctaacggtgactgcagccacttctgcttcccggtgcca
aatttccagcgagtgtgtgggtgcccttatggaatgaggctggcttccaatcacttgacatgcgagggggaaccaaccaatg
aaccacccacagagcagtgtggcttattttccttcccctgtaaaaatggcagatgtgtgcccaattactatctctgtgatggagt
cgatgacatgataacagtgatgagcaactatgtggcacacttaataatactgttcatcttcggcgttcacctgtggccat
ggggagtgcattcctgcacactggcgctgtgaaaaacgcaacgactgtgtggatggcagtgatgagcacaactgccccac
ccacgcacctgcttcctgccttgacacccaatacacctgtgataatcaccagtgtatctcaaagaactgggtctgtgacacag
acaatgattgtggggatggatctgatgaaaagaactgcaattcgacagagacatgccaacctagtcagtttaattgccccaat
catcgatgtattgacctatcgtttgtctgtgatggtgacaaggattgtgttgatggatctgatgaggttggttgtgtattaaactgt
actgcttctcaattcaagtgtgccagtggggataaatgtattggcgtcacaaatcgttgtgatggtgtttttgattgcagtgacaa
ctcggatgaagcaggctgtccaaccaggcctcctggtatgtgccactcagatgaatttcagtgccaagaagatggtatctgc
atcccgaacttctgggaatgtgatgggcatccagactgcctctatggatctgatgagcacaatgactgtgtccccaagacttg
cccttcatcatatttccactgtgacaacggaaactgcatccacagggcatggctctgtgatcgggacaatgactgcggggat
atgagtgatgagaaggactgccctactcagccctttcgctgtcctagttggcaatggcagtgtcttggccataacatctgtgtg
aatctgagtgtagtgtgtgatggcatctttgactgccccaatgggacagatgagtccccactttgcaatgggaacagctgctc
agatttcaatggtggttgtactcacgagtgtgttcaagagccctttggggctaaatgcctatgtccattgggattcttacttgcca
atgattctaagacctgtgaagacatagatgaatgtgatattctaggctcttgtagccagcactgttacaatatgagaggttctttc
cggtgctcgtgtgatacaggctacatgttagaaagtgatgggaggacttgcaaagttacagcatctgagagtctgctgttactt
gtggcaagtcagacaaattattgccgacagtgtcacctcccaggtccacaatatctattcattggtcgagaatggttcttac
attgtagctgttgattttgattcaattagtggtcgtatcttttggtctgatgcaactcagggtaaaacctggagtgcgtttcaaaat
ggaacggacagaagagtggtatttgacagtagcatcatcttgactgaaactattgcaatagattgggtaggtcgtaatctttac
tggacagactatgctctggaaacaattgaagtctccaaaattgatgggagccacaggactgtgctgattagtaaaaacctaa
caaatccaagaggactagcattagatcccagaatgaatgagcatctactgttctggtctgactggggccaccaccctcgcat
cgagcgagccagcatggacggcagcatgcgcactgtcattgtccaggacaagatcttctggccctgcggcttaactattga
ctaccccaacagactgctctacttcatggactcctatcttgattacatggacttttgtgattataatggacaccatcggagacag
gtgatagccagtgatttgattatacggcacccctatgccctaactctctttgaagactctgtgtactggactgaccgtgctactc
gtcgggttatgcgagccaacaagtggcatggagggaaccagtcagttgtaatgtataatattcaatggccccttgggattgtt
gcggttcatccttcgaacaaccaaattccgtgaatccatgtgccttttcccgctgcagccatctctgcctgctttcctcacagg
ggcctcatttttactcctgtgtttgtccttcaggatggagtctgtctcctgatctcctgaattgcttgagagatgatcaacctttctt
aataactgtaaggcaacatataatttttggaatctcccttaatcctgaggtgaagagcaatgatgctatggtccccatagcagg
gatacagaatggtttagatgttgaatttgatgatgctgagcaataaatctattgggttgaaaatccagggtgaaattcacagagt
gaagacagatggcaccaacaggacagtatttgcttctatatctatggtggggccttctatgaacctggccttagattggatttc
aagaaacctttattctaccaatcctagaactcagtcaatcgaggttttgacactccacggagatatcagatacagaaaaacatt
gattgccatgatgggacagctctggagttggcttccaattggcataactgttgatcctgctcgtgggaagctgtactggtc
agaccaaggaactgacagtggggttcctgccaagatcgccagtgctaacatggatggcacatctgtgaaaactctctttact
gggaacctcgaacacctggagtgtgtcactcttgacatcgaagagcagaaactctactgggcagtcactggaagaggaagt
gattgaaagaggaaacgtggatggaacagatcgaatgatcctggtacaccagctttcccacccctggggaattgcagtcca
tgattctttcctttattatactgatgaacagtatgaggtcattgaaagagttgataaggccactggggccaacaaaatagtcttg
agagataatgttccaaatctgaggggtcttcaagtttatcacagacgcaatgccgccgaatcctcaaatggctgtagcaacaa
catgaatgcctgtcagcagatttgcctgcctgtaccaggaggattgttttcctgcgcctgtgccactggatttaaactcaatcct
gataatcggtcctgctctccatataactctttcattgttgtttcaatgctgtctgcaatcagaggctttagcttggaattgtcagatc
attcagaaaccatggtgccggtggcaggccaaggacgaaacgcactgcatgtggatgtggatgtgtcctctggctttatttat
tggtgtgattttagcagctcagtggcatctgataatgcgatccgtagaattaaaccagatggatcttctctgatgaacattgtga
cacatggaataggagaaaatggagtccggggtattgcagtggattgggtagcaggaaatctttatttcaccaatgcctttgttt
ctgaaacactgatagaagttctgcggatcaatactacttaccgccgtgttcttcttaaagtcacagtggacatgcctaggcata
ttgtgtagatcccaagaacagatacctcttctgggctgactatgggcagagaccaaagattgagcgttctttccttgactgtac
caatcgaacagtgcttgtgtcagagggcattgtcacaccacggggcttggcagtggaccgaagtgatggctacgtttatta
gtgatgattctttagatataattgcaaggatcgtacaatggagagaactctgaagtgattcgttatggcagtcgttacccaac
tccttatggcatcactgtttttgaaaattctatcatatgggtagataggaatttgaaaaagatcttccaagccagcaaggaacca
gagaicacagagccacccacaggatagagacaaatcaactggctaagagatgtgaccatctttgacaagcaagtcca
gccccggtcaccagcagaggtcaacaacaacccttgcttggaaaacaatggtgggtgctctcatctctgctttgctctgcctg
gattgcacaccccaaaatgtgactgtgcctttgggaccctgcaaagtgatggcaagaattgtgccatttcaacagaaaacttc
ctcatctttgccttgtctaattccttgagaagcttacacttggaccctgaaaaccatagcccacctttccaaacaataaatgtgg
aaagaactgtcatgtctctagactatgacagtgtaagtgatagaatctacttcacacaaaatttagcctctggagttggacaga
tttcctatgccaccctgtcttcagggatccatactccaactgtcattgcttcaggtatagggactgctgatggcattgcctttgac
tggattactagaagaatttattacagtgactacctcaaccagatgattaattccatggctgaagatgggtctaaccgcactgtg
atagcccgcgttccaaaaccaagagcaattgtgttagatccctgccaagggtacctgtactgggctgactgggatacacatg
ccaaaatcgagagagccacattgggaggaaacttccgcgtacccattgtgaacagcagtctggtcatgcccagtgggctga
ctctggactatgaagaggaccttctctactgggtggatgctagtctgcagaggattgaacgcagcactctgacgggcgtgga
tcgtgaagtcattgtcaatgcagccgttcatgcttttggcttgactctctatggccagtatatttactggactgacttgtacacaca
aagaatttaccgagctaacaaatatgacgggtcaggtcagattgcaatgaccacaaatttgctctcccagcccaggggaatc
aacactgttgtgaagaaccagaaacaacagtgtaacaatccttgtgaacagtttaatgggggctgcagccatatctgtgcac
caggtccaaatggtgccgagtgccagtgtccacatgagggcaactggtatttggccaacaacaggaagcactgcattgtgg
acaatggtgaacgatgtggtgcatcttccttcacctgctccaatgggcgctgcatctcggaagagtggaagtgtgataatgac
aacgactgtggggatggcagtgatgagatggaaagtgtctgtgaacttcacacctgctcaccgacagccttcacctgtgcca
atgggcgatccatactcttaccgctagattactacaatgactgtggtgatggcagtgatgaggcagggtgcctgttc
agggactgcaatgccaccacggagtttatgtgcaataacagaaggtgcatacctcgtgagtttatctgcaatggtgtagaca
actgccatgataataacacttcagatgagaaaaattgccctgatcgcacttgccagtctggatacacaaaatgtcataattcaa
atatttgtattcctcgcgtttatttgtgtgacggagacaatgactgtggagataacagtgatgaaaaccctacttattgcaccact
cacacgtgcagcagcagtgagttccaatgcgcatctgggcgctgtattcctcaacattggtattgtgatcaagaaacagattg
ttttgatgcctctgatgaacctgcctcttgtggtcactctgagcgaacatgcctagctgatgagttcaagtgtgatggtgggag
gtgcatcccaagcgaatggatctgtgaaggtgataatgactgtggggatatgagtgacgaggataaaaggcaccagtgtca
gaatcaaaactgctcggattacgagtttctctgtgtaaatgacagacctccggacaggaggtgcattccccagtcttgggtct
gtgatggcgatgtggattgtactgacggctacgatgagaatcagaattgcaccaggagaacttgctctgaaaatgaattcac
ctgtggttacggactgtgtatcccaaagatattcaggtgtgaccggcacaatgactgtggtgactatagcgacgagaggggc
tgcttataccagacttgccaacagaatcagtttacctgtcagaacgggcgctgcattagtaaaaccttcgtctgtgatgaggat
aatgactgtggagacggatctgatgagctgatgcacctgtgccacaccccagaacccacgtgtccacctcacgagttcaag
tgtgacaatgggcgctgcatcgagatgatgaaactctgcaaccacctagatgactgtttggacaacagcgatgagaaaggc
tgtggcattaatgaatgccatgaaccacaatcagtggctgcgatcacaactgcacagacaccttaacaagtttctattgttcct
gtcgtcctggttacaagctcatgtctgacaagcggacttgtgttgatattgatgaatgcacagagatgccttttgtctgtagcca
gaagtgtgagaatgaataggctcctacatctgtaagtgtgccccaggctacctccgagaaccagatggaaagacctgccg
gcaaaacagtaacatcgaaccctatctcatttttagcaaccgttactatttgagaaatttaactatagatggctatttttactccct
catcttggaaggactggacaatgtggcattagattttgaccgagtagagaagagattgtattggattgatacacagaggc
aagtcattgagagaatgtttctgaataagacaaacaaggagacaatcataaaccacagactaccagctgcagaaagtctgg
ctgtagactgggtttccagaaagctctactggttggatgcccgcctggatggcctctttgtctctgacctcaatggtggacacc
gccgcatgctggcccagcactgtgtggatgccaacaacaccttctgctttgataatcccagaggacttgcccttcaccctcaa
tatgggtacctctactgggcagactggggtcaccgcgcatacattgggagagtaggcatggatggaaccaacaagtctgtg
ataatctccaccaagttagagtggcctaatggcatcaccattgattacaccaatgatctactctactgggcagatgcacacatg
ggttacatagagtactctgatttggagggccaccatcgacacacggtgtatgatggggcactgcctcaccctttcgctattac
cattttgaagacactatttattggacagattggaatacaaggacagtggaaaagggaaacaaatatgatggatcaaatagac
agacactggtgaacacacacacagaccatttgacatccatgtgtaccatccatataggcagcccattggtgagcaatccct
gtggtaccaacaatggtggctgttctcatctctgcctcatcaagccaggaggaaaagggttcacttgcgagtgtccagatga
cttccgcacccttcagctgagtggcagcacctactgcatgcccatgtgctccagcacccagttcctgtgcgctaacaatgaa
aagtgcattcctatctggtggaaatgtgatggacagaaagactgctcagatggctctgatgaactggccctttgcccgcagc
gcttctgccgactgggacagttccagtgcagtgacggcaactgcaccagcccgcagactttatgcaatgctcaccaaaattg
ccctgatgggtctgatgaagaacgtattctttgtgagaatcaccactgtgactccaatgaatggcagtgcgccaacaaacgtt
gcatcccagaatcctggcagtgtgacacatttaacgactgtgaggataactcagatgaagacagttcccactgtgccagcag
gacctgccggccgggccagtttcggtgtgctaatggccgctgcatcccgcaggcctggaagtgtgatgtggataatgattgt
ggagaccactcggatgagcccattgaagaatgcatgagctctgcccatctctgtgacaacttcacagaattcagctgcaaaa
caaattaccgctgcatcccaaagtgggccgtgtgcaatggtgtagatgactgcagggacaacagtgatgagcaaggctgtg
aggagaggacatgccatcctgtgggggatttccgctgtaaaaatcaccactgcatccctcttcgttggcagtgtgatgggca
aaatgactgtggagataactcagatgaggaaaactgtgctccccgggagtgcacagagagcgagtttcgatgtgtcaatca
gcagtgcattccctcgcgatggatctgtgaccattacaacgactgtggggacaactcagatgaacgggactgtgagatgag
gacctgccatcctgaatattttcagtgtacaagtggacattgtgtacacagtgaactgaaatgcgatggatccgctgactgttt
ggatgcgtctgatgaagctgattgtcccacacgctttcctgatggtgcatactgccaggctactatgttcgaatgcaaaaacc
atgtttgtatcccgccatattggaaatgtgatggcgatgatgactgtggcgatggttcagatgaagaacttcacctgtgcttgg
atgttccctgtaattcaccaaaccgtttccggtgtgacaacaatcgctgcatttatagtcatgaggtgtgcaatggtgtggatga
ctgtggagatggaactgatgagacagaggagcactgtagaaaaccgacccctaaaccttgtacagaatatgaatataagtg
tggcaatgggcattgcattccacatgacaatgtgtgtgatgatgccgatgactgtggtgactggtccgatgaactgggttgca
ataaaggaaaagaaagaacatgtgctgaaaatatatgcgagcaaaattgtacccaattaaatgaaggaggatttatctgctcc
tgtacagatgggttcgaaaccaatgtttttgacagaacctcctgtctagatatcaatgaatgtgaacaatttgggacttgtcccc
agcactgcagaaataccaaaggaagttatgagtgtgtctgtgctgatggcttcacgtctatgagtgaccgccctggaaaacg
atgtgcagctgagggtagctctcctttgttgctactgcctgacaatgtccgaattcgaaaatataatctctcatctgagaggttct
cagagtatcttcaagatgaggaatatatccaagctgttgattatgattgggatcccaaggacataggcctcagtgttgtgtatta
cactgtgcgaggggagggctctaggtttggtgctatcaaacgtgcctacatccccaactttgaatccggccgcaataatcttg
tgcaggaagttgacctgaaactgaaatacgtaatgcagccagatggaatagcagtggactgggttggaaggcatatttactg
gtcagatgtcaagaataacgcattgaggtggataaacttgatggaaggtacagaaagtggctgatttccactgacctggac
caaccagctgctattgctgtgaatcccaaactagggctatgttctggactgactggggaaaggaacctaaaatcgagtctgc
ctggatgaatggagaggaccgcaacatcctggttttcgaggaccttggttggccaactggcctttctatcgattatttgaacaa
tgaccgaatctactggagtgacttcaaggaggacgttattgaaaccataaaatatgatgggactgataggagagtcattgca
aaggaagcaatgaacccttacagcctggacatctttgaagaccagttatactggatatctaaggaaaagggagaagtatgg
aaacaaaataaatttgggcaaggaaagaaagagaaaacgctggtagtgaacccttggctcactcaagttcgaatctttcatc
aactcagatacaataagtcagtgcccaacctttgcaaacagatctgcagccacctctgccttctgagacctggaggatacag
ctgtgcctgtccccaaggctccagctttatagaggggagcaccactgagtgtgatgcagccatcgaactgcctatcaacctg
ccccccccatgcaggtgcatgcacggaggaaattgctattttgatgagactgacctccccaaatgcaagtgtcctagcggct
acaccggaaaatattgtgaaatggcgttttcaaaaggcatctctccaggaacaaccgcagtagctgtgctgttgacaatcctc
ttgatcgtcgtaattggagctctggcaattgcaggattcttccactatagaaggaccggctcccttttgcctgctctgcccaagc
tgccaagcttaagcagtctcgtcaagccctctgaaaatgggaatggggtgaccttcagatcaggggcagatcttaacatgga
tattggagtgtctggttttggacctgagactgctattgacaggtcaatggcaatggtgaagactttgtcatggaaatggggaag
cagcccataatatttgaaaacccaatgtactcagccagagacagtgctgtcaaagtggttcagccaatccaggagactgtatc
tgaaaatgtggatataagaattatggaagtcccataaacccttctgagatagttccagagacaaacccaacttcaccagctg
ctgatggaactcaggtgacaaaatggaatctcttcaaacgaaaatctaaacaaactaccaactttgaaaatccaatctatgca
cagatggagaacgagcaaaaggaaagtgttgctgcgacaccacctccatcaccttcgctccctgctaagcctaagcctcctt
cgagaagagacccaactccaacctattctgcaacagaagacacttttaaagacaccgcaaatcttgttaaagaagactctga
agtatagctatacaagctatttagggaataattagaaacacacttttgcacatatattttttacaaaaagatgaaaaaagttaaca
ttcagtactttatgaaaaaaatatatttttccctgtttgcctatagttggaggtatcctgtgtgtctttttttacttatgccgtctcatatt
ttacaaataattatcacaatgtactatatgtatatctttgcactgaagttgtctgaaggtaatactataaatatattgtatatttgtaa
attttggaaagattatcctgttactgaatttgctaataaagatgtctgctgatttggttggtgatcattatagtaaatgatccaacaa
gaaaaggaattgactgggg
DEFINITION Homo sapiens low density lipoprotein receptor-related protein 3
(LRP3), mRNA.
ACCESSION XM_036573 SEQ ID NO:26
Tacgggctgcaccgccaccgcacaaagacgcctcgggagccgccgcctgcacccgggccgcagcagccacgccag
ccggagcccgagccctagcccgagcccgagcccgagccgcagccagagccagagaccgcagccggaaaagcccg
gctggagggcgcgccgggcgcccgggcgcagctggccacgtatgtctggtgacatctttctcaccgggagatcagc
agtgcggttcctgccttagcggcctgcagtgggaagctggagcagcacacggagcggcgtggggtcatctacagcccgg
cctggcccctcaactacccgccaggcaccaactgcagctggtacatccagggcgaccgtggtgacatgattaccatcagct
tccgcaactttgacgtggaggagtcccaccagtgctccctggactggctcctgctgggcccagcagccccaccccgccag
gaggccttccgcctctgtggctccgccatcccacctgccttcatctctgcccgcgaccatgtctggattttcttccactcagac
gcctccagctccggccaggcacagggcttccgtctgtcttacatccgagggaagctgggccaggcatcctgccaggcaga
tgagttccgctgtgacaacggcaagtgcctgcccggcccgtggcagtgcaacacggtggacgagtgtggagacggctct
gatgagggcaactgctcggcgcccgcctccgagcctccaggcagcctgtgccccggggggaccttcccatgcagcggg
gcgcgctccacgcgctgcctgcctgtggagcggcgctgtgacggcttgcaggactgcggcgacggctcggatgaggcg
ggctgccccgacctggcgtgcggccggcggctgggcagcttctacggctcctttgcctccccagacctgttcggcgccgc
tcgcgggccctcagaccttcactgcacgtggctggtggacacacaggactcccggcgggtgctgctgcagctggaactgc
ggctgggctatgacgactacgtgcaggtatacgagggcctgggcgagcgcggggaccgcc,tgctgcagacgctgtccta
ccgcagcaaccaccggcccgtgagcctggaggccgcccagggccgcctcactgtggcctaccacgcgcgcgcccgca
gcgccggccacggcttcaatgccacctaccaggtgaagggctattgcctcccctgggagcagccgtgcgggagcagtag
tgacagtgacgggggaagcctgggcgaccagggctgcttctcagagccacagcgctgtgatggctggtggcatagtgcca
gcggccgagacgagcagggctgccctgcctgcccgcccgaccagtacccctgcgagggtggcagtggtctgtgctacac
gcctgccgaccgctgcaacaaccagaaaagctgtcccgacggcgccgacgagaagaactgcttctcctgccagaccggc
accttccactgcggtaccaacctgtgcatcttcgagacgtggcgctgtgacggccaggaagactgccaggacggcagcga
tgagcatgggtgcctggccgccgtgccccgcaaggtcatcacggcggcgctcattggcagcctggtgtgtggcctgctgc
tggtcatcgcgctgggctgcgccttcaagctctactcactgagcacgcaggaatacagggccttcgagacccagatgacgc
gcctggaggctgagttcgtgcggcgggaggcacccccatcctatggtcagctcatcgcccagggcctcattccacccgtg
gaggactttcctgtctacagtgcgtcccaggcctctgtgctgcagaatcttcgcacagccatgcggagacagatgcgtcggc
acgcctcccgccgggggccctcccgccgccgcctcggccgcctctggaaccggctctttcaccggccgcgggcgcccc
gaggccagatcccactgctgaccgcagcacgcccatcacagaccgtgctgggcgatggcttcctccagcctgctccagg
ggctgcccccgaccccccagcaccgctcatggacacaggcagcaccagggcggccggagacaggccccccagtgccc
ccggccgtgcaccggaggtgggaccttcagggccacccttgccctcgggcctgcgagacccagagtgcaggcccgtgg
acaaggacagaaaggtctgcagggagccactggtagacggcccagctcctgcagatgcacctcgggagccctgctcag
cccaggacccgcacccccaggtctccactgccagcagcaccctgggcccccactcgccagagccactgggggtctgca
ggaaccccccgcccccctgctcaccaatgctggaggccagcgatgatgaggccctgttggtctgttgaccgctgggctcg
DEFINITION Homo sapiens low density lipoprotein receptor-related protein 4
(LRP4), mRNA.
ACCESSION XM_035037 SEQ ID NO:27
Caggcatgaacagtttcctcatcttcgccaggaggatagacattcgcatggtctccctggacatcccttattttgctgatgtgg
tggtaccaatcaacattaccatgaagaacaccattgccattggagtagacccccaggaaggaaaggtgtactggtctgaca
gcacactgcacaggatcagtcgtgccaatctggatggctcacagcatgaggacatcatcaccacagggctacagaccaca
gatgggctcgcggttgatgccattggccggaaagtatactggacagacacgggaacaaaccggattgaagtgggcaacct
ggacgggtccatgcggaaagtgttggtgtggcagaaacttgacagtccccgggccatcgtactgtaccatgagatggggtt
tatgtactggacagactggggggagaatgccaagttagagcggtccggaatggatggctcagaccgcgcggtgctcatca
acaacaacctaggatggcccaatggactgactgtggacaaggccagctcccaactgctatgggccgatgcccacaccga
gcgaattgaggctgctgacctgaatggtgccaatcggcatacattggtgtcaccggtgcagcacccatatggcctcaccctg
ctcgactcctatatctactggactgactggcagactcggagcatccaccgtgctgacaagggtactggcagcaatgtcatcc
tcgtgaggtccaacctgccaggcctcatggacatgcaggctgtggaccgggcacagccactaggttttaacaagtgcggct
cgagaaatggcggctgctcccacctctgcttgcctcggccttctggcttctcctgtgcctgccccactggcatccagctgaag
ggagatgggaagacctgtgatccctctcctgagacctacctgctcttctccagccgtggctccatccggcgtatctcactgga
caccagtgaccacaccgaitgcatgtccctgttcctgagctcaacaatgtcatctccctggactatgacagcgtggatgga
aaggtctattacacagatgtgttcctggatgttatcaggcgagcagacctgaacggcagcaacatggagacagtgatcggg
cgagggctgaagaccactgacgggctggcagtggactgggtggccaggaacctgtactggacagacacaggtcgaaat
accattgaggcgtccaggctggatggttcctgccgcaaagtactgatcaacaatagcctggatgagccccgggccattgct
gttttccccaggaaggggtacctcttctggacagactggggccacattgccaagatcgaacgggcaaacttggatggttctg
agcggaaggtcctcatcaacacagacctgggttggcccaatggccttaccctggactatgatacacgcaggatctactggg
tggatgcgcatctggaccggatcgagagtgctgacctcaatgggaaactgcggcaggtcttggtcagccatgtgtcccacc
cctttgccctcacacagcaagacaggtggatctactggacagactggcagaccaagtcaatccagcgtgttgacaaatact
caggccggaacaaggagacagtgctggcaaatgtggaaggactcatggatatcatcgtggtttcccctcagcggcagaca
gggaccaatgcctgtgtgaacaatggtggatgcacccacctctgctttgccagagcctcggacttcgtatgtgcctgtcc
tgacgaacctgatagccggccctgctcccttgtgcctggcctggtaccaccagctcctagggctactggaatgagtgaaaa
gagcccagtgctacccaacacaccacctaccaccttgtattcttcaaccacccggacccgcacgtctctggaggaggtgga
aggaagatgctctgaaagggatgccaggctgggcctctgtgcacgttccaatgacgctgttcctgctgctccaggggaagg
acttcatatcagctacgccattggtggactcctcagtattctgctgattttggtggtgattgcagctttgatgctgtacagacaca
aaaaatccaagttcactgatcctggaatggggaacctcacctacagcaacccctcctaccgaacatccacacaggaagtga
agattgaagcaatccccaaaccagccatgtacaaccagctgtgctataagaaagagggagggcctgaccataactacacc
aaggagaagatcaagatcgtagagggaatctgcctcctgtctggggatgatgctgagtgggatgacctcaagcaactgcg
aagctcacgggggggcctcctccgggatcatgtatgcatgaagacagacacggtgtccatccaggccagctctggctccc
tggatgacacagagacggagcagctgttacaggaagagcagtctgagtgtagcagcgtccatactgcagccactccagaa
agacgaggctctctgccagacacgggctggaaacatgaacgcaagctctcctcagagagccaggtctaaatgcccacatt
ctcttccctgcctgcctgttccttctcctttatggacgtctagtccttgtgctcgcttacaccgcaggccccgcttctgtgtgcttg
tcctcctcctcctcccaccccataactgttcctaagccttcaccggagctgtttaccacgtgagtccataactacctgtgcaca
agaatgatggcacatcacgagaatttagacctggattttaccatgaacctcacatcttgtactccatcctgggccccctgaaa
ctgcttattcgtgattcctcaccagcgagagctccacctcccctttccccagtaccctaagtgcctgcttctcagtgctgatgc
agctgatgacccaggactgcgctctgccacatcacagccagcatgactgcttctctgagagaacttgcccatcaggggctg
ggacatgggggtgtgggtaaagacagggatgaaggatagaggctgagagaagaaggaagaatcagcccagcaggtatg
ggcatctgggaaacctccagcctcaagtgtgttagtaacatgaaaaagctttggggggtagttggatctgggtgtctggtcca
ttgctggcagtggacattattcttgccctaagagacactgccttttcagcagcagatactggtgagatgggggtggctcaggc
tgttcttcctcctcctagaatgtctggagctgtttctacattcagataactgggtcccctatcacaaggctactggctaataggaa
ttccctcctggtgccaccactggccagtacctttcctaagtctttgctcaaattaaccaggttgtgagccagtggcttgagtgaa
tgttaggccttgggggctgagtctctgaaaagtctaagaagctctgcctagaccaaatatggtatacctcctgacccctctctc
cctcatgtcctgggattctggggaagagacctagaaacaagctttcaaagaaaaaccagaagttgtcataaatggtcagaaa
gaacgatcaggttggagacttgggaaacccagggcctaaagagaagtatccatgagggtcaaacttcctgttgaacttccta
tgttctttctcaagtgctcagggatctaagttagtggacagcaagcctgtggctacggggtggtgatgttcctcttccagctgtc
ccctcagctaaggggcttagtttccatgtgggatgccatcacttggttcatgctcattcacacaaagggcacgtgtctcagcct
ggtatcagggaaattgagacttatttttgccctaaaacgtctccctagctgttcttcgtggggtttttttgtttgtttttttgcctaattt
gctttttctgaccaagccttgtggcaccagcaatctccaaagtcctgtggtgggagggctgaataaataaaaatacaaagag
gtgggtaaggagtaggaaggtagagagcaccactgatgaggccctcctagcccatggcagacccagacctcttctccccc
aggaattagaagtggcaggagagaacaacaggggctgggaatggaggggagaatttctaggggaagtttcctgagttgaa
acttctcctgtggttactggtattgagaaatcagctaccaaagtgaaaaaggacaagatcaattcttttctagtcagttctaaga
ctgctagagagagataccaggccctagccttgctctcagtagcgtcagccccagttctgagcctccccacattacacttaac
aagcagtaaaggagtgagcactttgggtccttagactcacgtctggggaggaagagcaagtagaaaagtggcattttcttga
ttggaaagggggaaggatcttattgcacttgggctgttcagaatgtagaaaggacatatttgaggaagtatctatttgagcact
gatttactctgtaaaaagcaaaatctctctgtcctaaactaatggaagcgattctcccatgctcatgtgtaatggttttaacgttaac
tcactggagagattggacttctggagttatttaaccactatgttcagtattttaggactttatgataatttaatataaatttagctttt
cttaatc
DEFINITION Homo sapiens low density lipoprotein receptor-related protein 5
(LRP5), mRNA.
ACCESSION NM_002335 SEQ 31) NO:28
Atggagcccgagtgagcgcggcgcgggcccgtccggccgccggacaacatggaggcagcgccgcccgggccgccg
tggccgctgctgctgctgctgctgctgctgctggcgctgtgcggctgcccggcccccgccgcggcctcgccgctcctgcta
ttgccaaccgccgggaccggctggtggacgccggcggagtcaagctggagtccaccatcgtggtcagcggcctgg
aggatgcggccgcagtggacttccagttttccaagggagccgtgtactggacagacgtgagcgaggaggccatcaagca
gacctacctgaaccagacgggggccgccgtgcagaacgtggtcatctccggcctggtctctcccgacggcctcgcctgcg
actgggtgggcaagaagctgtactggacggactcagagaccaaccgcatcgaggtggccaacctcaatggcacatcccg
gaaggtgctcttctggcaggaccttgaccagccgagggccatcgccttggaccccgctcacgggtacatgtactggacag
actggggtgagacgccccggattgagcgggcagggatggatggcagcacccggaagatcattgtggactcggacattta
ctggcccaatggactgaccatcgacctggaggagcagaagctctactgggatgacgccaagctcagcttcatccaccgtg
ccaacctggacggctcgttccggcagaaggtggtggagggcagcctgacgcaccccttcgccctgacgctctccgggga
cactctgtactggacagactggcagacccgctccatccatgcctgcaacaagcgcactggggggaagaggaaggagatc
ctgagtgccctctactcacccatggacatccaggtgctgagccaggagcggcagcctttcttccacactcgctgtgaggag
gacaatggcggctgatcccacctgtgcctgctgtccccaagcgagcctttctacacatgcgcctgccccacgggtgtgcag
ctgcaggacaacggcaggacgtgtaaggcaggagccgaggaggtgctgctgctggcccggcggacggacctacggag
gatctcgctggacacgccggactttaccgacatcgtgctgcaggtggacgacatccggcacgccattgccatcgactacga
cccgctagagggctatgtctactggacagatgacgaggtgcgggccatccgcagggcgtacctggacgggtctggggcg
cagacgctggtcaacaccgagatcaacgaccccgatggcatcgcggtcgactgggtggcccgaaacctctactggaccg
acacgggcacggaccgcatcgaggtgacgcgcctcaacggcacctcccgcaagatcctggtgtcggaggacctggacg
agccccgagccatcgcactgcaccccgtgatgggcctcatgtactggacagactggggagagaaccctaaaatcgagtgt
gccaacttggatgggcaggagcggcgtgtgctggtcaatgcctccctcgggtggcccaacggcctggccctggacctgc
aggaggggaagctctactggggagacgccaagacagacaagatcgaggtgatcaatgttgatgggacgaagaggcgga
ccctcctggaggacaagctcccgcacattttcgggttcacgctgctgggggacttcatctactggactgactggcagcgccg
cagcatcgagcgggtgcacaaggtcaaggccagccgggacgtcatcattgaccagctgcccgacctgatggggctcaaa
gctgtgaatgtggccaaggtcgtcggaaccaacccgtgtgcggacaggaacggggggtgcagccacctgtgcttcttcac
accccacgcaacccggtgtggctgccccatcggcctggagctgctgagtgacatgaagacctgcatcgtgcctgaggcct
tcttggtcttcaccagcagagccgccatccacaggatctccctcgagaccaataacaacgacgtggccatcccgctcacgg
gagtcaaggaggcctcagccctggactttgatgtgtccaacaaccacatctactggacagacgtcagcctgaagaccatca
gccgcgccttcatgaacgggagctcggtggagcacgtggtggagtttggccttgactaccccgagggcatggccgttgac
tggatgggcaagaacctctactgggccgacactgggaccaacagaatcgaagtggcgcggctggacgggcagttccgg
caagtcctcgtgtggagggacttggacaacccgaggtcgctggccctggatcccaccaagggctacatctactggaccga
gtggggcggcaagccgaggatcgtgcgggcctcatggacgggaccaatgcatgacgcttgacaaggtgggccg
ggccaacgacctcaccattgactacgctgaccagcgcctctactggaccgacctggacaccaacatgatcgagtcgtcca
acatgctgggtcaggagcgggtcgtgattgccgacgatctcccgcacccgttcggtctgacgcagtacagcgattatatcta
ctggacagactggaatctgcacagcattgagcgggccgacaagactagcggccggaaccgcaccctcatccagggcca
cctggacttcgtgatggacatcctggtgttccactcctcccgccaggatggcctcaatgactgtatgcacaacaacgggcag
tgtgggcagatgtgccttgccatccccggcggccaccgctgcggctgcgcctcacactacaccctggaccccagcagcc
gcaactgcagcccgcccaccaccttcttgctgttcagccagaaatctgccatcagtcggatgatcccggacgaccagcaca
gcccggatctaatcctgcccctgcatggactgaggaacgtcaaagccatcgactatgacccactggacaagttcatctactg
ggtggatgggcgccagaacatcaagcgagccaaggacgacgggacccagccctttgttttgacctctctgagccaaggcc
aaaacccagacaggcagccccacgacctcagcatcgacatctacagccggacactgttctggacgtgcgaggccaccaa
accatcaacgtccacaggctgagcggggaagccatgggggtggtgctgcgtggggaccgcgacaagcccagggccat
ctcgtcaacgcggagcgagggtacctgtacttcaccaacatgcaggaccgggcagccaagatcgaacgcgcagccctg
gacggcaccgagcgcgaggtcctcttcaccaacggcctcatccgccctgtggccctggtggtagacaacacactgggca
agctgttctgggtggacgcggaactgaagcgcattgagagctgtgacctgtcaggggccaaccgcctgacactggagga
cgccaacatcgtgcagcctctgggcctgaccatccttggcaagcatctctactggatcgaccgacagcagcagatgatcga
gcgtgtggagaagaccaccggggacaagcggactcgcatccagggccgtgtcgcccacctcactggcatccatgcagtg
gaggaagtcagcctggaggagttctcagcccacccatgtgcccgtgacaatggtggctgctcccacatctgtattgccaag
ggtgatgggacaccacggtgctcatgcccagtccacctcgtgctcctgcagaacctgctgacctgtggagagccgcccac
ctgctccccggaccagtttgcatgtgccacaggggagatcgactgtatccccggggcctggcgctgtgacggctttcccga
gtgcgatgaccagagcgacgaggagggctgccccgtgtgctccgccgcccagttcccctgcgcgcggggtcagtgtgtg
gacctgcgcctgcgctgcgacggcgaggcagactgtcaggaccgctcagacgaggcggactgtgacgccatctgcctgc
ccaaccagttccggtgtgcgagcggccagtgtgtcctcatcaaacagcagtgcgactccttccccgactgtatcgacggct
ccgacgagctcatgtgtgaaatcaccaagccgccctcagacgacagcccggcccacagcagtgccatcgggcccgtcat
tggcatcatcctctctctcttcgtcatgggtggtgtctattttgtgtgccagcgcgtggtgtgccagcgctatgcgggggccaa
cgggcccttcccgcacgagtatgtcagcgggaccccgcatgcccctcaatttcatagccccgggcggttcccagcatg
gccccttcacaggcatcgcatgcggaaagtccatgatgagctccgtgagcctgatggggggccggggcggggtgcccct
ctacgaccggaaccacgtcacaggggcctcgtccagcagctcgtccagcacgaaggccacgctgtacccgccgatcctg
aacccgccgccctccccggccacggacccctccctgtacaacatggacatgttctactcttcaaacattccggccactgtga
gaccgtacaggccctacatcattcgaggaatggcgcccccgacgacgccctgcagcaccgacgtgtgtgacagcgacta
cagcgccagccgctggaaggccagcaagtactacctggatttgaactcggactcagacccctatccacccccacccacgc
cccacagccagtacctgtcggcggaggacagctgcccgccctcgcccgccaccgagaggagctacttccatctcttcccg
ccccctccgtccccctgcacggactcatcctgacctcggccgggccactctggcttctctgtgcccctgtaaatagttttaaat
atgaacaaagaaaaaaatatattttatgatttaaaaaataaatataattgggattttaaaaacatgagaaatgtgaactgtgatgg
ggtgggcagggctgggagaactttgtacagtggaacaaatatttataaacttaattttgtaaaacag
DEFINITION Homo sapiens low density lipoprotein receptor-related protein 6
(LRP6), Mrna.
ACCESSION NM_002336 SEQ ID NO:29
Gcggccgccccggctcctcgcctcccccacttctggccacccctcgccggtgagagaagagaacgcgagaagggaag
atgggggccgtcctgagagagcctcctggcctgcagcttctgtgtgctcctgagacggcccctttgttgctttatgcaaacag
acgggacttgcgattggttgatgctacaaatggcaaagagaatgctacgattgtagttggaggcttggaggatgcagctgcg
gtggactttgtgtttagtcatggcttgatatactggagtgatgtcagcgaagaagccattaaacgaacagaatttaacaaaact
gagagtgtgcagaatgttgttgtttctggattattgtcccccgatgggctggcatgtgattggcttggagaaaaattgtactgga
cagattctgaaactaatcggattgaagtttctaatttagatggatctttacgaaaagttttattttggcaagagttggatcaaccca
gagctattgccttagatcctttcaagtgggttcatgtactggacagactggggagaagtgccaaagatagaacgtgctggaat
ggatggttcaagtcgcttcattataataaacagtgaaatttactggccaaatggactgactttggattatgaagaacaaaagctt
tattgggcagatgcaaaacttaatttcatccacaaatcaaatctggatggaacaaatcggcaggcagtggttaaaggttccctt
ccacatccttttgccttgacgttatttgaggacatattgtactggactgactggagcacacactccattttggcttgcaacaagta
tactggtgagggtctgcgtgaaatccattctgacatcttctctcccatggatatacatgccttcagccaacagaggcagccaa
atgccacaaatccatgtggaattgacaatgggggttgttcccatttgtgtttgatgtctccagtcaagcctttttatcagtgtgctt
gccccactggggtcaaactcctggagaatggaaaaacctgcaaagatggtgccaaagaattattgcttttagctcgaagga
cagacttgagacgcatttctttggatacaccagattttacagacattgttctgcagttagaagacatccgtcatgccattgccat
agattacgatcctgtggaaggctacatctactggactgatgatgaagtgagggccatacgccgttcatttatagatggatctg
gcagtcagtttgtggtcactgctcaaattgcccatcctgatggtattgctgtggactgggttgcacgaaatctttattggacaga
cactggcactgatcgaatagaagtgacaaggctcaatgggaccatgaggaagatcttgatttcagaggacttagaggaacc
ccgggctatagataccatggttgggtacatgtattggactgactggggagaaattccgaaaattgagcgagcagctc
tggatggttctgaccgtgtagtattggttaacacttctcttggttggccaaatggtttagccttggattatgatgaaggcaaaata
tactggggagatgccaaaacagacaagattgaggttatgaatactgatggcactgggagacgagtactagtggaagacaa
aattcctcacatatttggatttactttgttgggtgactatgtttactggactgactggcagaggcgtagcattgaaagagttcata
aacgaagtgcagagagggaagtgatcatagatcagctgcctgacctcatgggcctaaaggctacaaatgttcatcgagtga
ttggttccaacccctgtgctgaggaaaacgggggatgtagccatctctgcctctatagacctcagggccttcgctgtgcttgc
cctattggctttgaactcatcagtg&aatgaagacctgcattgtcccagaggctttccttttgttttcacggagagcagatatca
gacgaatttctctggaaacaaacaaataatgtggctattcactcactggtgtcaaagaagcttctgctttggattttgatgtg
acagacaaccgaatttattggactgatatatcactcaagaccatcagcagagcctttatgaatggcagtgcactggaacatgt
ggtagaattcggcttagattatccagaaggcatggcagtagactggcttgggaagaacttgtactgggcagacacaggaac
gaatcgaattgaggtgtcaaagttggatgggcagcaccgacaagttttggtgtggaaagacctagatagtcccagagctctc
gcgttggaccctgccgaaggatttatgtattggactgaatggggtggaaaacctaagatagacagagctgcaatggatgga
agtgaacgtactaccttagttccaaatgtggggcgggcaaacggcctaactattgattatgctaaaaggaggctttattggac
agacctggacaccaacttaatagaatcttcaaatatgcttgggctcaaccgtgaagttatagcagatgacttgcctcatcctttt
ggcttaactcagtaccaagattatatctactggacggactggagccgacgcagcattgagcgtgccaacaaaaccagtggc
caaaaccgcaccatcattcagggccatttggattatgtgatggacatcctcgtctttcactcatctcgacagtcagggtggaat
gaatgtgcttccagcaatgggcactgctcccacctctgcttggctgtgccagttgggggttttgtttgtggatgccctgcccac
tactctcttaatgctgacaacaggacttgtagtgctcctacgactttcctgctcttcagtcaaaagagtgccatcaaccgcatgg
tgattgatgaacaacagagccccgacatcatccttcccatccacagccttcggaatgtccgggccattgactatgacccact
ggacaagcaactctattggattgactcacgacaaaacatgatccgaaaggcacaagaagatggcagccagggctttactgt
gtgtgagctcagttccgagtcagaacctggaaatacaaccctatgacctcagcattgatatttacagccgctacatctactg
gacgaggctaccaatgtcattaatgtgacaagattagatgggagatcagttggagtggtgctgaaaggcgagcagga
cagacctcgagccattgtggtaaaaccagagaaagggtatatgtattttaccaatcttcaggaaaggtctcctaaaattgaac
gggctgctttggatgggacagaacgggaggtcctctttttcagtggcttaagtaaacaaattgctttagcccttgatagcaggc
tgggcaagctcttttgggctgattcagatctccggcgaattgaaagcagtgatctctcaggtgctaaccggatagtattagaa
gactccaatatcttgcagcctgtgggacttactgtgtttgaaaactggctctattggattgataaacagcagcaaatgattgaaa
aaattgacatgacaggtcgagagggtagaaccaaagtccaagctcgaattgcccagcttagtgacattcatgcagtaaagg
agctgaaccttcaagaatacagacagcaccctgctcaggataatggtggctgttcacatatttgtcttgtaaagggggatg
gtactacaaggtgttcttgccccatgcacctggttctacttcaagatgagctatcatgtggagaacctccaacatgttctcctca
gcagtttacttgtttcacgggggaaattgactgtatccctgtggcttggcggtgcgatgggtttactgaatgtgaagaccacag
tgatgaactcaattgtcctgtatgctcagagtcccagttccagtgtgccagtgggcagtgtattgatggtgccctccgatgcaa
tggagatgcaaactgccaggacaaatcagatgagaagaactgtgaagtgctttgtttaattgatcagttccgctgtgccaatg
gtcagtgcattggaaagcacaagaagtgtgatcataatgtggattgcagtgacaagtcagatgaactggattgttatccgact
gaagaaccagcaccacaggccaccaatacagttggttctgttattggcgtaattgtcaccatttttgtgtctggaactgtatact
ttatctgccagaggatgttgtgtccacgtatgaagggagatggggaaactatgactaatgactatgtagttcatggaccagctt
ctgtgcctcttggttatgtgccacacccaagttctttgtcaggatctcttccaggaatgtctcgaggtaaatcaatgatcagctc
cctcagtatcatggggggaagcagtggacccccctatgaccgagcccatgttacaggagcatcatcaagtagttcttcaag
caccaaaggcacttacttccctgcaattttgaaccctccaccatccccagccacagagcgatcacattacactatggaatttg
gatattcttcaaacagtccttccactcataggtcatacagctacaggccatatagctaccggcactttgcaccccccaccaca
ccctgcagcacagatgtttgtgacagtgactatgctcctagtcggagaatgacctcagtggcaacagccaagggctatacc
agtgacttgaactatgattcagaacctgtgcccccacctcccacaccccgaagccaatacttgtcagcagaggagaactatg
aaagctgcccaccttctccatacacagagaggagctattctcatcacctctacccaccgccaccctctccctgtacagactcc
tcctgaggaggggccctcctcctctgactgcctccaataaaaatgtaaatataaatttggttgagatctggaggggggga
gggagctatagagaaggatgaggcagaccatgtacagttaaaattataaaatggggtagggaatactggagatatttgtac
agaagaaaggatatttatatattttcttaaaacagcagatttgctgcttgtgccataaaagtttgtataaaaaaaatttgtactaa
aagttttatttttgcaaactaaatacacaaagcatgccttaaacccagtgaagcaactgagtacaaaggaaacaggaataata
aaggcatcactgaccaggaatatctgggctttattgataccaaaaaaaaaaaaa
DEFINITION Homo sapiens low density lipoprotein receptor-related protein 8,
aploipoprotein receptor (LRP8), mRNA.
ACCESSION XM_016537 SEQ ID NO:30
Tgctgctgctgctgctgctgctgctgctgcagctccagcatcttgcggcggcagcggctgatccgctgctcggcggccaa
gggccggccaaggattgcgaaaaggaccaattccagtgccggaacgagcgctgcatcccctctgtgtggagatgcgacg
aggacgatgactgcttagaccacagcgacgaggacgactgccccaagaagacctgtgcagacatgactcagctagac
aacggacactgcatccacgaaaggtggaagtgtgattggcgaggaggagtgtcctgatggctccgatgagtccgaggcca
cttgcaccaagcaggtgtgtcctgcagagaagctgagctgtggaaccaccagccacaaggtacctgcctcacgct
gcgacggggagaaggactgcgagggtggagccggatgaggccggctgtgctacctcactgggcacctgccaagga
cgaaccaagtggggatgggacatgcctgcgatcagcactgcacaggagcaggactaccagatgggaag
atgaagctggctgcctacaggggctgaacgagtgtctgcacgacaatggcggctgctcacacatctgcactgacctcaga
ttggctttgaatgcacgtgcccagcaggcttccagctcctggaccagaagagctgtggcgacattgatgagtgcaaggacc
cagatgcctgcagccagatctgtgtcaattacaagggctattttaagtgtgagtgctaccctggctacgagatggacctactg
accaagaactgcaaggctgctggtggcaagagcccatccctaatcttcaccaaccggcacgaggtgcggaggatcgacct
ggtgaagcggaactattcacgcctcatccccatgctcaagaatgtcgtggcactagatgtggaagttgccaccaatcgcatc
tactggtgtgacctctcctacagtaagatctatagcgcctacatggacaaggccagtgacccgaaagagcaggaggtcctc
attgacgagcagttgcactctccagagggcctggcagtggactgggtccacaagcacatctactggactgactcgggcat
aagaccatctcagtggccacagttgatggtggccgccgacgcactctcttcagccgtaacctcagtgaaaccccgggccatc
gctgttgaccccctgcgagggttcatgattggtctgactggggggaccaggccaagattgagaatctgggctcacga
gtggaacggcaaacactggtgtcagacaatattgaatggcccaacggaatcaccctggatctgctgagccagcgcttgtac
tgggtagactccaagctacaccaactgtccagcattgacttcagtggaggcaacagaaagacgctgatctcctccactgact
tcctgagccacccttttgggatagctgtgtttgaggacaaggtctggacagacctggagaacgaggccattcaagca
aatcggctcaatggcctggaaatctccatcctggctgagaacctcaacaacccacatgaaattgtcatattccatgagctgaa
gcagccaagagctccagatgcctgtgagctgagtgtccagcctaatggaggctgtgaatacctgtgccttcctgatcctcag
atctccagccactctcccaagtacacatgtgcctgtcctgacacaatgtggctgggtccagacatgaagaggtgctaccgag
cacctcaatctacctcaactacgacgttagcttctaccatgacgaggacagtacctgccaccacaagagaccccgggacca
ccgtccacagatccacctaccagaaccacagcacagagacaccaagcctgacagctgcagtcccaagctcaaaaac
cccagggctcccagcatcagcccgtctacccggccctgcaaccagcaaccactcccagcactatgcaatgagacag
taagatgggctcaacagtcactgccgctgttatcgggatcatcgtgcccatagtggtgatagccctcctgtgcatgagtggat
aactgatctggagaactggaagcggaagaacaccaaaagcatgaattttgacaacccagtctacaggaaaacaacagga
gaagaagacgaagatgagctccatatagggagaactgctcagattggccatgtctatcctgcagcaatcagcagcagatc
gcccactgtgggcagagccctgtcttggggagaccagagaaccggaagacccagccactgccctcaggagcttagac
ttgccgggggaaccaaggtcacagctgcaccaactcccgaagaaccctctttccgagctgcctgtcatcaaatccaagcga
gtggcattaagccttgaagatgatggactaccctgaggatgggatcacccccttcgtgcctcatggaattcagtcccatgcac
tacactctggatggtgtatgactggatgaatgggtttctatatatgggtctgtgtgagtgtatgtgtgtgtgtgattttttttttatt
tatgttgcggaaaggtaaccacaaagttatgatgaactgcaaacatccaaaggatgtgagagtttttctatgtataatgttttata
cactttttaactggttgcactacccatgaggaattcgtggaatggctactgctgactaacatgatgcacataaccaaatgggg
gccaatggcacagtaccttactcatcatttaaaaactatatttacagaagatgtttggttgctgggggggcttttttaggttttgg
ggcatttgttttttgtaaataagatgattatgctttgtggctatccatcaacataagtaaaaaacacttcaactcc
ctcccccatttagattatttattaacatattttaaaaatcagatgagtctataaataatttagagaagtgagagtatttataggc
atgtttggcccaccacacagactctgtgtgtgtatgtgtgtgtttatatgtgtatgtgtgtgacagaaaaatctgaaagagagag
gcacatctatggctactgttcaaatacataaagataaatttattttcacacagtccacaaggggatatcttgtagttttcagaa
agcctttggaaatctggatcagaaaatagataccatggtttgtgcaattatgtagtaaaaaaggcaaatcttttcacctctggct
attcctgagaccccaggaagtcaggaaaagcctttcagctcacccatggctgctgtgactcctaccagggctttcttggcttt
ggcgaaggtcagtgtacagacattccatggtaccagagtgctcagaaactcaagataggatatgcctcaccctcagctactc
cttgttttaaagttcagctctttgagtaacttcttcaatttctttcaggacacttgggttgaattcagtaagtttcctctgaagcaccc
tgaagggtgccatccttacagagctaagtggagacgtttccagatcagcccaagtttactatagagactggcccaggcactg
aatgtctaggacatgctgtggatgaagataaagatggtggaataggttttatcacatctcttatttctcttttccccttactctctac
catttcctttatgtggggaaacattttaaggtaataaataggttacttaccatcatatgttcatatagatgaaactaatttttgctta
agtcagaacaactggccaaaattgaagtcatatttgaggggggaaatggcatacgcaatattatattatattggatatttatatt
cacacaggaatttggtttactgctttgtaaataaaagaaaaactccgggtatatgt
DEFINITION Homo sapiens porcupine (MG61), mRNA.
ACCESSION NM_022825 SEQ ID NO:31
Gcaatggccacctttagccgccaggaatttttccagcagctactgcaaggctgtctcctgcctactgcccagcagggccttg
accagatctggctgctccttgccatctgcctcgcctgccgcctcctctggaggctcgggttgccatcctacactgaagcatgca
agcaccgtggcaggcgggtcttcagcctctaccacttcttccagctgcaaatggtttgggtcgtgctgctcagcctcctgtg
ctacctcgtgctgtcctctgccgacattcctcccatcgaggcgtcttcctatccgtcaccatcctcatctacctatcatgggig
agatgcacatggagacaccagacatggcacaagaagcgaggggcacagatgaggccatgaggcagatctctg
ggcttcgacctggaccggggcgaggtgggtacggtgccctcgccagtggagttcatgggctacctctacttcgtgggcac
catcgtcttcgggccctggatatccttccacagctacctacaaactgtacaaggccgcccactgagctgccggtggctgcag
aaggtggcccggagcctggcactggccctgctgtgccttgtgctgtccacttgcgtgggcccctacctcucccaacucat
ccccctcaacggtgaccgcctccttcgcaacaagaaacgcaaagccaggggcaccatggtaaggtggctgcgagcctac
gagagtgctgtctccttccacttcagcaactagggcwctttccgaggccacggccacgttggcgggggctggca
accgaggagaaggatcacctggaatgggacctgacggtgtccaagccactgaatgtggagctgcctcggtcatggtgga
agttgtcacaagctggaacctgcccatgtcttattggctaaataactatgttttcaagaatgctctccgcctggggaccttctcg
gctgtgctggtcacctatgcagccagcgccctcctacatggcttcagtttccacctggctgcggtcctgctgtccctggctttt
atcacttacgtggagcatgtcctccggaagcgcctggctcggatcctcagtgcctgtgtcttgtcaaagcggtgcacgccag
actgttcgcaccagcatcgcttgggcctgggggtgcgagccttaaacttgctctttggagctctggccatcttccacctggcc
tacctgggctccctgtttgatgtcgatgtggatgacaccacagaggagcagggctacggcatggcatacactgtccacaagt
ggtcagagctcagctgggccagtcactgggtcacttttggatgctggatcttctaccgtctcataggctgaggcaaatctgtg
gaccctcataaccctcttaagacccctctcagggtgccactgatgggggatgagggaaggccctctctctactccugaccc
cctccatccttgacccccaacacctcaacacacacacacacacacacacacaaaatcacaaccattttcatgcctgtcaatcc
ccaccccaacacaaggcaggaagggggtggtgcctgctggggcctagagggggatgcttggggaacagagagggg
agatccagggcctccccgccttccttcttcctttttatatacaatttgttattgtcaaataaaagtaggaaatattcaataggct
Extracellular Wnt antagonists:
DEFINITION Homo sapiens dickkopf (Xenopus laevis) homolog 1 (DKK1), mRNA.
ACCESSION XM_048687 SEQ ID NO:32
Cggcacggtttcgtggggacccaggcttgcaaagtgacggtcattttctctttctttctccctcttgagtccttctgagatgatg
gctctgggcgcagcgggagctacccgggtctttgtcgcgatggtagcggcggctctcggcggccaccctctgctgggagt
gagcgccaccttgaactcggttctcaattccaacgctatcaagaacctgcccccaccgctgggcggcgctgcggggcacc
caggctctgcagtcagcgccgcgccgggaatcctgtacccgggcgggaataagtaccagaccattgacaataccagcc
gtacccgtgcgcagaggacgaggagtgcggcactgatgagtactgcgctagtcccacccgcggaggggacgcaggcgt
gcaaatctgtctcgcctgcaggaagcgccgaaaacgctgcatgcgtcacgctatgtgctgccccgggaattactgcaaaaa
tggaatatgtcttctgatcaaaatcatttccgaggagaaattgaggaaccatcactgaaaagctttggaatgatcatagc
accttggatgggtattccagaagaaccaccttgtcttcaaaaatgtatcacaccaaaggacaagaaggttctgtttgtctccgg
tcatcagactgtgcctcaggattgtgttgtgctagacacttctggtccaagatctgtaaacctgtcctgaaagaaggtcaagtg
tgtaccaagcataggagaaaaggctctcatggactagaaatattccagcgttgttactgtggagaaggtctgtcttgccggat
acagaaagatcaccatcaagccagtaattcttctaggcttcacaaatgtcagagacactaaaccagctatccaaatgcagtga
actccttttatataatagatgctatgaaaaccttttatgaccttcatcaactcaatcctaaggatatacaagttctgtggtttcagtt
aagcattccaataacaccttccaaaaacctggagtgtaagagctttgtttctttatggaactcccctgtgattgcagtaaattact
gtattgtaaattctcagtgtggcacttacctgtaaatgcaatgaaacttttaattaattctaaaggtgctgcactgcctatttttcct
cttgttatgtaaatttttgtacacattgattgttatcttgactgacaaatattctatattgaactgaagtaaatcatttcagcttatagtt
cttaaaagcataaccctttaccccatttaattctagagtctagaacgcaaggatctcttggaatgacaaatgataggtacctaaa
atgtaacatgaaaatactagcttattttctgaaatgtactatcttaatgcttaaattatatttccctttaggctgtgatagtttttgaaat
aaaatttaacatttaatatcatgaaatgttataagtagacat
DEFINITION Homo sapiens dickkopf (Xenopus laevis) homolog 2 (DKK2), mRNA.
ACCESSION XM_052645 SEQ ID NO:33
Gcgggtctcgcttgggttccgctaactgtcctgaggcgtgagatgagacatagggtcctgggtccccgaaccaggaa
gggttgagggaacacaatctgcaagcccccgcgaaccaagtgaggggccccggttggggtcctccctccctttgcattcc
cacccctccgggctttgcgtcttcctggggaccccctcgccgggagatggccgcgttgatgcggagcaaggattcgtcctg
ctgcctgctcctactggccgcggtgctgatggtggagagctcacagatcggcagttcgcgggccaaactcaactccatcaa
gtcctctctgggcggggagacgcctggtcaggccgccaatcgatctgcgggcatgtaccaaggactggcattcggcggc
agagaagggcaaaacctggggcaggcctaccctgtagcagtgataaggagtgtgaagttgggaggtattgccacag
tccccaccaaggatcatcggcctgcatggtgtgtcggagaaaaaagaagcgctgccaccgagatggcatgtgctgcccca
gtacccgctgcaataatggcatctgtatcccagttactgaaagcatcttaacccctcacatcccggctctggatggtactcgg
cacagagatcgaaaccacggtcattactcaaaccatgacttgggatggcagaatctaggaagaccacacactaagatgtca
catataaaagggcatgaaggagacccctgcctacgatcatcagactgcattgaagggttttgctgtgctcgtcatttctggac
caaaatctgcaaaccagtgctccatcagggggaagtctgtaccaaacaacgcaagaagggttctcatgggctggaaattttc
cagcgttgcgactgtgcgaagggcctgtcttgcaaagtatggaaagatgccacctactcctccaaagccagactccatgtgt
gtcagaaaatttgatcaccattgaggaacatcatcaattgcagactgtgaagttgtgtatttaatgcattatagcatggtggaaa
ataaggttcagatgcagaagaatggctaaaataagaaacgtgataagaatatagatgatcac
DEFINITION Homo sapiens dickkopf (Xenopus laevis) homolog 3 (DKK3), mRNA.
ACCESSION XM_006030 SEQ ID NO:34
Ctatcacaatgagaccaacacagacacgaaggttggaaataataccatccatgtgcaccgagaaattcacaagataacca
acaaccagactggacaaatggtcttttcagagacagttatcacatctgtgggagacgaagaaggcagaaggagccacgag
tgcatcatcgacgaggactgtgggcccagcatgtactgccagtttgccagcttccagtacacctgccagccatgccggggc
cagaggatgctctgcacccgggacagtgagtgctgtggagaccagctgtgtgtctggggtcactgcaccaaaatggccac
caggggcagcaatgggaccatctgtgacaaccagagggactgccagccggggctgtgctgtgccttccagagaggcctg
ctgttccctgtgtgcacacccctgcccgtggagggcgagctttgccatgaccccgccagccggcttctggacctcatcacct
gggagctagagcctgatggagccttggaccgatgcccttgtgccagtggcctcctctgccagccccacagccacagcctg
gtgtatgtgtgcaagccgaccttcgtggggagccgtgaccaagatggggagatcctgctgcccagagaggtccccgatga
gtatgaagttggcagcttcatggaggaggtgcgccaggagctggaggacctggagaggagcctgactgaagagatggcg
ctgggggagcctgcggctgccgccgctgcactgctgggaggggaagagatttagatctggaccaggctgtgggtagatgt
gcaatagaaatagctaatttatttccccaggtgtgtgctttaggcgtgggctgaccaggcttcttcctacatcttcttcccagtaa
gtttcccctctggcttgacagcatgaggtgttgtgcatttgttcagctcccccaggctgttctccaggcttcacagtctggtgctt
gggagagtcaggcagggttaaactgcaggagcagtttgccacccctgtccagattattggctgctttgcctctaccagttgg
cagacagccgtttgttctacatggctttgataattgtttgaggggaggagatggaaacaatgtggagtctccctctgattggttt
tggggaaatgtggagaagagtgccctgctttgcaaacatcaacctggcaaaaatgcaacaaatgaattttccacgcagttctt
tccatgggcataggtaagctgtgccttcagctgttgcagatgaaatgttctgttcaccctgcattacatgtgtttattcatccagc
agtgttgctcagctcctacctctgtgccagggcagcattttcatatccaagatcaattccctctctcagcacagcctggggagg
gggtcaUgttctcctcgtccatcagggatctcagaggnctcagagactgcaagctgcttgcccaagtcacacagctagtga
agaccagagcagtttcatctggttgtgactctaagctcagtgctctctccactaccccacaccagccttggtgccaccaaaag
tgctccccaaaaggaaggagaatgggatttttcttttgaggcatgcacatctggaattaaggtcaaactaattctcacatccct
ctaaaagtaaactactgttaggaacagcagtgttctcacagtgtggggcagccgtccttctaatgaagacaatgatattgaca
ctgtccctctttggcagttgcattagtaactttgaaaggtatatgactgagcgtagcatacaggttaacctgcagaaacagtact
taggtaattgtagggcgaggattataaatgaaatttgcaaaatcacttagcagcaactgaagacaattatcaaccacgtggag
aaaatcaaaccgagcagggctgtgtgaaacatggttgtaatatgcgactgcgaacactgaactctacgccactccacaaat
gatgttttcaggtgtcatggactgttgccaccatgtattcatccagagttcttaaagtttaaagttgcacatgattgtataagcatg
ctttctttgagttttaaattatgtataaacataagttgcatttagaaatcaagcataaatcac
DEFINITION Homo sapiens dickkopf (Xenopus laevis) homolog 4 (DKK4), mRNA.
ACCESSION XM_032444 SEQ ID NO:35
Agacgacgtgctgagctgccagcttagtggaagctctgctctgggtggagagcagcctcgctttggtgacgcacagtgct
gggaccctccaggagccccgggattgaaggatggtggcggccgtcctgctggggctgagctggctctgctctcccctggg
agctctggtcctggacttcaacaacatcaggagctctgctgacctgcatggggcccggaagggctcacagtgcctgtctga
cacggactgcaataccagaaagctgcctccagccccgcgatgagaagccgttctgctacatgcgtgggcgga
gaggtgccagcgagatgccatgtgctgccctgggacactctgtgtgaacgatgtttgtactacgatggaagatgcaacccc
aatattagaaaggcagcttgatgagcaagatggcacacatgcagaaggaacaactgggcacccagtccaggaaaaccaa
cccaaaaggaagccaagtattaagaaatcacaaggcaggaagggacaagagggagaaagttgtctgagaacttttgactg
tggccctggactttgctgtgctcgtcatttttggacgaaaatttgtaagccagtccttttggagggacaggtctgctccagaag
agggcataaagacactgctcaagctccagaaatcttccagcgttgcgactgtggccctggactactgtgtcgaagccaattg
accagcaatcggcagcatgctcgattaagagtatgccaaaaaatagaaaagctataaatatttcaaaataaagaagaatcca
cattgc
DEFINITION Human Frizzled related protein Frzb precursor (fzrb) mRNA,
complete cds
ACCESSION U24163 SEQ ID NO:36
Acggggcctgggcggsaggggcggtggctggagctcggtaaagctcgtgggaccccattgggggaatttgatccaagg
aagcggtgattgccgggggaggagaagctcccagatccttgtgtccacttgcagcgggggaggcggagacgcggagcg
ggccttttggcgtccactgcgcggctgcaccctgccccatcctgccgggatcatggtctgcggcagcccgggagggatgc
tgctgctgcgggccgggctgcttgccctggctgctctctgcctgctccgggtgcccggggctcgggctgcagcctgtgagc
ccgtccgcatccccctgtgcaagtccctgccctggaacatgactaagatgcccaaccacctgcaccacagcactcaggcc
aacgccatcctggccatcgagcagttcgaaggtctgctgggcacccactgcagccccgatctgctcttcttcctctgtgccat
gtacgcgcccatctgcaccattgacttccagcacgagcccatcaacccctgtaagtctgtgtgcgagcgggcccggcagg
gctgtgagcccatactcatcaagtaccgccactcgtggccggagaacctggcctgcgaggagctgccagtgtacgacagg
ggcgtgtgcatctctcccgaggccatcgttactgcggacggagctgattttcctatggattctagtaacggaaaatgtagagg
ggcaagcagtgaacgctgtaaatgtaagcctattagagctacacagaagacctatttccggaacaattacaactatgtcattc
gggctaaagttaaagagataaagactaagtgccatgatgtgactgcagtagtggaggtgaaggagattctaaagtcctctct
ggtaaacattccacgggacactgtcaacctctataccagctctggctgcctctgccctccacttaatgttaatgaggaatatat
catcatgggctatgaagatgaggaacgttccagattactcttggtggaaggctctatagctgagaagtggaaggatcgactc
ggtaaaaaagttaagcgctgggatatgaagcttcgtcatcttggactcagtaaaagtgattctagcaatagtgattccactcag
agtcagaagtctggcaggaactcgaacccccggcaagcacgcaactaaatcccgaaatacaaaaagtaacacagtggac
ttcctattaagacttacttgcattgctggactagcaaaggaaaattgcactattgcacatcatattctattgtttactataaaaatca
tgtgataactgattattacttctgtttctcttttggtttctgcttctctcttctctcaacccctttgtaatggtttgggggcagactctta
agtatattgtgagttttctatttcactaatcatgagaaaaactgttcttttgcaataataataaattaaacatgctgtta
gagtcgcacccagcgaagagagcgggcccgggacaagctcgaactccggccgcctcgcccttaaccagctccgtccct
ctaccccctaggggtcgcgcccacgatgctgcagggccctggctcgctgctgctgctcttcctcgcctcgcactgctgcctg
ggctcggcgcgcgggctcttcctctttggccagcccgacttctcctacaagcgcagcaattgcaagcccatcccggccaac
ctgcagctgtgccacggcatcgaataccagaacatgcggctgcccaacctgctgggccacgagaccatgaaggaggtgc
tggagcaggccggcgcttggatcccgctggtcatgaagcagtgccacccggacaccaagaagttcctgtgctcgctcttcg
cccccgtctgcctcgatgacctagacgagaccatccagccatgccactctcgntgcgtgcaggtgaaggatcgctgcgcc
ccggtcatgtccgccttcccctggcccgacatgcttgagtgcgaccgtttcccccaggacaacgacctttgcatccccctcg
ctagcagcgaccacctcctgccagccaccgaggaagctccaaaggtatgtgaagcctgcaaaaataaaaatgatgatgac
aacgacataatggaaacgctttgtaaaaatgattttgcactgaaaataaaagtgaaggagataacctacatcaaccgt
Cytoplasmic acting components:
DEFINITION Homo sapiens adenomatosis polyposis coli (APC), mRNA.
ACCESSION XM_043933 SEQ ID NO:38
Aggtccaagggtagccaaggatggctgcagcttcatatgatcagttgttaaagcaagttgaggcactgaagatggagaact
caaatcttcgacaagagctagaagataattccaatcatcttacaaaactggaaactgaggcatctaatatgaaggaagtactt
aaacaactacaaggaagtattgaagatgaagctatggcttcttctggacagattgatttattagagcgtcttaaagagcttaact
tagatagcagtaatttccctggagtaaaactgcggtcaaaatgtccctccgttcttatggaagccgggaagggatctatca
agccgttctggagagtgcagtcctgttcctatgggttcatttccaagaagagggtttgtsaaatggaagcagagaaagtactgg
atatttagaagaacttgagaaagagaggtcattgcttcttgctgatcttgacaaagaagaaaaggaaaaagactggtattacg
ctcaacttcagaatctcactaaaagaatagatagtcttcctttaactgaaaatttttccttacaaacagatatgaccagaaggca
attggaatatgaagcaaggcaaatcagagttgcgatggaagaacaactaggtacctgccaggatatggaaaaacgagcac
agcgaagaatagccagaattcagcaaatcgaaaaggacatacttcgtatacgacagcttttacagtcccaagcaacagaag
cagagaggtcatctcagaacaagcatgaaaccgg6tcacatgatgctgagcggcagatgaaggacaaggaagggaga
aatcaacatggcaacttctggtaatggtcagggttcaactacacgaatggaccatgaaacagccagtgttttgagttctagta
gcacacactctgcacctcgaaggctgacaagtcatctgggaaccaaggtggaaatggtgtattcattgttgtcaatgcttggt
actcatgataaggatgatatgtcgcgaactttgctagctatactagctcccagacagctatatccatgcgacaactgga
tgtcttcctctcctcatccagcttttacatggcaatgacaagactctgtauagggaauccaggggcagagaggctc
gggccagggccagtgcagcactccacaacatcattcactcacagcctgatgacaagagaggcaggcgtgaaatccgagt
ccttcatcttttggaacagatacgcgcttactgtgaaacctgttgggagtggcaggaagctcatgaaccaggcatggaccag
gacaaaaatccaatgccagctcctgttgaacatcagatctgtcctgctgtgtgtgttctaatgaaactttcatttgatgaagagc
atagacatgcaatgaatgaactagggggactacaggccattgcagaattattgcaagtggactagaaataatgggcact
aatgaccactacagtattacactaagacgatatgctggaatggcttgacaaacttgaaggagatgtagccacaaaggct
acgctatgctctatgaaaggctgcatgagagcacttgtggcccaactaatctgagtgaagacttacagcagaaatga
gagtgttttgaggaatttgtcttggcgagcagatgtaaatagtaaaaagacgttgcgagaagttggaagtgtgaaagcattga
tggaatgtgctttagaagttaaaaaggaatcaaccctcaaaagcgtattgagtgccttatggatttacagcacattgcaatg
agaataaagctgatatatgtgctgtagatggtgcacttgcatttttggttggcactcttacttaccggagccagacaaacacttt
agccattattgaaagtggaggtgggatattacggaatgtgtccagcttgatagctacaaatgaggaccacaggcaaatccta
agagagaacaactgtctacaaactttattacaacacttaaaatctcatagtttgacaatagtcagtaatgcatgtggaactttgtg
gaatctctcagcaagaaatcctaaagaccaggaagcattatgggacatgggggcaaagcatgctcagagctcattgat
tcaaagcacaaaatgattgctatgggaagtgctgcagctttaaggaatctcatggcaaataggcctgcgaagtacaaggatg
ccaatattatgtctcctggctcaagcttgccatctcttcatgttaggacaagccctagaagcagattagatgctcagc
aatcagaaactttgacaatatagacaataagtcccaaggcatctcatcaaaagcagagacacaagcaagactct
atggtgattatgtgacaccaatcgacatgatgataataggtcagacaaaaatactggcaacatgactaccacaccat
gagagaacgcggaattggtctaggcaactaccatccagcaacagaatccaggaacncttcagcgagagcagat
ctccaccactgcagcccagattgccaaagtcatggagaagtgtcagccattcatacctctcaggaagacagaattctgg
gtctaccactgaattacattgtgtgacagatgagagaatgcaauagaagagctctgctgcccatacacaucaaaacacia
caatttcactaagtcggaaaattcaaataggacatguctatgccttatgccagaatacaagagatcttcaaatgataa
gtaagttttgcagttatggtcaatacccagccgacctagcccataaatacataagcatcatatggatgaaatgatggag
aactagatacaccaataaattatagtctaaatatcagatgagcatgactctggaggcccacacagaatgaa
gatgggcaagacccaaacacataatagaagatgaaataaaacaaagtgagcaaagacaatcaaggaatcaaagtacaact
tatcctgtttatactgagagcactgatgataaacacctcaagttccaaccacattttggacagcaggaatgtgtttctccataca
ggtcacggggagccaatggttcagaaacaaatcgagtgggttctaatcatggaattaatcaaaatgtaagccagtctttgtgt
caagaagatgactatgaagatgataagcctaccaattatagtgaacgttactctgaagaagaacagcatgaagaagaagag
agaccaccaaattatagcataaaatataatgaagagaaacgtcatgatcagccgaaatagtttaaatatgccaca
gatattccttcatcacagaaacagtcattttcattctcaaagagttcatctggacaaagcagtaaaaccgaacatatgtcttcaa
gcagtgagaatacgtccacaccttcatctaatgccaagaggcagatcagctccatcaaaactgcacagaaagaagtg
gtcagcctcaaaaggctgccatgcaaagucttctattaacaagacacagacttaaacaagatactccaaat
atgtttttcaagatgtagttcattatcatctttgtcatcagctgaagatgataggattcagacaacacaggaaagcagat
tctgctaataccctgcaaatagcagaaataaaaagaaaaagattggaactaggtcagctgaagatcctgtgagcgaagttccag
cagtgtcacagcaccatagaaccaaatccagcagactgcagggttctagtttatcttcagaatcagccaggcacaaagctgt
tgaattttcttcaggagcgaaatctccctccaaaagtggtgctcagacacccaaaagtccacctgaacactatgttcaggaga
ccccactcatgtttagcagatgtacttctgtcagttcacttgatagttttgagagtcgttcgattgccagctccgttcagagtgaa
ccatgcagtggaatggtaagtggcattataagccccagtgatcttccagatagccctggacaaaccatgccaccaagcaga
agtaaaacacctccaccacctcctcaaacagctcaaaccaagcgagaagtacctaaaaataaagcacctactgctgaaaag
agagagagtggacctaagcaagctgcagtaaatgctgcagttcagagggtccaggttcttccagatgctgatactttattaca
ttttgccacggaaagtactccagatggattttcttgttcatccagcctgagtgctctgagcctcgatgagccatttatacagaaa
gatgtggaattaagaataatgcctccagttcaggaaaatgacaatgggaatgaaacagaatcagagcagcctaaagaatca
aatgaaaaccaagagaaagaggcagaaaaaactattgattctgaaaaggacctattagatgattcagatgatgatgatattga
aatactagaagaatgtattatttctgccatgccaacaaagtcatcacgtaaagcaaaaaagccagcccagactgcttcaaaat
tacctccacctgtggcaaggaaaccaagtcagctgcctgtgtacaaacttctaccatcacaaaacaggttgcaaccccaaaa
gcatgttagttttacaccgggggatgatatgccacgggtgtattgtgttgaagggacacctataaacttttccacagctacatct
ctaagtgatctaacaatcgaatcccctccaaatgagttagctgctggagaaggagttagaggaggggcacagtcaggtgaa
tttgaaaaacgagataccattcctacagaaggcagaagtacagatgaggctcaaggaggaaaaacctcatctgtaaccata
cctgaattggatgacaataaagcagaggaaggtgatattcttgcagaatgcattaattctgctatgcccaaagggaaaagtca
caagcctttccgtgtgaaaaagataatggaccaggtccagcaagcatctgcgtcttcttctgcacccaacaaaaatcagttag
atggtaagaaaaagaaaccaacttcaccagtaaaacctataccacaaaatactgaatataggacacgtgtaagaaaaaatg
cagactcaaaaaataatttaaatgctgagagagttttctcagacaacaaagattcaaagaaacagaatttgaaaaataattcca
aggtcttcaatgataagctcccaaataatgaagatagagtcagaggaagttttgcttttgattcacctcatcattacacgcctatt
gaaggaactccttactgtttttcacgaaatgattctttgagttctctagattttgatgatgatgatgttgacctttccagggaaaag
gctgaattaagaaaggcaaaagaaaataaggaatcagaggctaaagttaccagccacaaagaactaacctccaaccaaca
atcagctaataagacacaagctattgcaaagcagccaataaatcgaggtcagcctaaacccatacttcagaaacaatccact
tttccccagtcatccaaagacataccagacagaggggcagcaactgatgaaaagttacagaattttgctattgaaaatactcc
ggtttgcttttctcataattcctctctgagttctctcagtgacattgaccaagaaaacaacaataaagaaaatgaacctatcaaa
gagactgagccccctgactcacagggagaaccaagtaaacctcaagcatcaggctatgctcctaaatcatttcatgttgaag
ataccccagtttgtttctcaagaaacagttctctcagttctcttagtattgactctgaagatgacctgttgcaggaatgtataagct
ccgcaatgccaaaaaagaaaaagccttcaagactcaagggtgataatgaaaaacatagtcccagaaatatgggtggcatat
taggtgaagatctgacacttgatttgaaagatatacagagaccagattcagaacatggtctatcccctgattcagaaaattttg
attggaaagctattcaggaaggtgcaaattccatagtaagtagtttacatcaagctgctgctgctgcatgtttatctagacaagc
ttcgtctgattcagattccatcctttccctgaaatcaggaatctctctgggatcaccatttcatcttacacctgatcaagaagaaa
aaccatttacaagtaataaaggcccacgaattctaaaaccaggggagaaaagtacattggaaactaaaaagatagaatctg
aaagtaaaggaatcaaaggaggaaaaaaagtttataaaagtttgattactggaaaagttcgatctaattcagaaatttcaggc
caaatgaaacagccccttcaagcaaacatgccttcaatctctcgaggcaggacaatgattcatattccaggagttcgaaatag
ctcctcaagtacaagtcctgtttctaaaaaaggcccaccccttaagactccagcctccaaaagccctagtgaaggtcaaaca
gccaccacttctcctagaggagccaagccatctgtgaaatcagaattaagccctgttgccaggcagacatcccaaataggt
gggtcaagtaaagcaccttctagatcaggatctagagattcgaccccttcaagacctgcccagcaaccattaagtagaccta
tacagtctcctggccgaaactcaatttcccctggtagaaatggaataagtcctcctaacaaattatctcaacttccaaggacat
catcccctagtactgcttcaaataagtcctcaggttctggaaaaatgtcatatacatctccaggtagacagatgagccaacag
accttaccaaacaacaggtttatccaagaatgccagtagtattccaagaagtgagtctgcctccaaaggactaaatcagat
gaataatggtaatggagccaataaaaaggtagaactttctagaatgtcttcaactaaatcaagtggaagtgaatctgatagatc
agaaagacctgtattagtacgccagtcaactttcatcaaagaagctccaagcccaaccttaagaagaaaattggaggaatct
gcttcatttgaatctctttctccatcatctagaccagcttctcccactaggtcccaggcacaaactccagttttaagtccttccctt
cctgatatgtctctatccacacattcgtctgacaggctggtggatggcgaaaactcccactatctcagtcccactatagagt
ataatgatggaagaccagcaaagcgccatgatattgcacggtctcattctgaaagtccttctagacttccaatcaataggtca
ggaacctggaaacgtgagcacagcaaacattcatcatcccttcctcgagtaagcacttggagaagaactggaagttcatctt
caattctttctgcttcatcagaatccagtgaaaaagcaaaaagtgaggatgaaaaacatgtgaactctatttcaggaaccaaa
caaagtaaagaaaaccaagtatccgcaaaaggaacatggagaaaaataaaagaaaatgaattttctcccacaaatagtactt
ctcagaccgtttcctcaggtgctacaaatggtgctgaatcaaagactctaatttatcaaatggcacctgctgtttctaaaacaga
ggatgtttgggtgagaattgaggactgtcccattaacaatcctagatctggaagatctcccacaggtaatactcccccggtga
ttgacagtgtttcagaaaaggcaaatccaaacattaaagattcaaaagataatcaggcaaaacaaaatgtgggtaatggcag
tgtcccatgcgaccgtgggtttggaaaatcgcctgaactcctttattcaggtggatgaccctgaccaaaaaggaactgaga
taaaaccaggacaaaataatcctgtccctgtatcagagactaatgaagttctatagtggaacgtaccccattcagttctagca
gctcaagcaaacacagttcacctagtgggactgttgctgccagagtgactccttttaattacaacccaagccctaggaaaag
cagcgcagatagcacttcagctcggccatctcagatcccaactccagtgaataacaacacaaagaagcgagattccaaaa
ctgacagcacagaatccagtggaacccaaagtcctaagcgccattctgggtcttaccttgtgacatctgtttaaaagagagg
aagaatgaaactaagaaaattctatgttaattaca2ctgctatatagacattttgtttcaaatgaaactttaaaagactgaaaaatt
ttgtaaataggtttgattcttgttagagggtttttgttctggaagccatatttgatagtatactttgtcttcactggtcttattttggga
ggcactcttgatggttaggaaaaaaatagtaaagccaagtatgtttgtacagtatgttttacatgtatttaaagtagcatcccatc
ccaacttcctttaatattgcttgtcttaaataatgaazactacagatagaaaatatgatatattgctgttatcaatcatttctagat
tataaactgactaaacttacatcagggaaaaattggtatttatgcaaaaaaaaatgtttttgtccttgtgagtccatctaacatcat
aattaatcatgtggctgtgaaattcacagtaatatggttcccgatgaacaagtttacccagcctgctttgctttactgcatgaatg
aaactgatggttcaatttcagaagtaatgattaacagttatgtggtcacatgatgtgcatagagatagctacagtgtaataattta
cactattttgtgctccaacaaaacaaaaatctgtgtaactgtaaaacattgaatgaaactattttacctgaactagattttatctg
aaagtaggtagaatttttgctatgctgtaatttgttgtatattctggtatttgaggtgagatggctgctcttttattaatgagacatga
attgtgtctcaacagaaactaaatgaacatttcagaataaattattgctgtatgtaaactgttactgaaattggtatttgtttgaagg
gtcttgtttcacatttgtattaataattgtttaaaatgcctcttttaaaagcttatataaatttttttcttcagcttctatgcattaagagta
aaattcctcttactgtaataaaaacaattgaagaagactgttgccacttaaccattccatgcgttggcacttatctattcctgaaat
ttcttttatgtgattagctcatcttgatttttaatatttttccacttaaacttt-tttttcttactccactggagctcagtaaaagtaaattca
tgtaatagcaatgcaagcagcctagcacagactaagcattgagcataataggcccacataatttcctctttcttaatattataga
attatgtacttgaaattgattcttagacattgcagtctcttcgaggctttacagtgtaaactgtcttgccccttcatcttcttgttgca
actgggtctgacatgaacactttttatcaccctgtatgttagggcaagatctcagcagtgaagtataatcagcactttgccatga
tcagaaaattcaaatcacatggaactttagaggtagatttaatacgattaagatattcagaagtatattttagaatccctgcctgtt
aaggaaactttatttgtggtaggtacagttctggggtacatgttaagtgtccccttatacagtggagggaagtcttccttcctga
aggaaaataaactgacacttattaactaagataatttacttaatatatcttccctgatttgttttaaaagatcagagggtgactgat
gatacatgcatacatatttgttgaataaatgaaaatttatttttagtgataagattcatacactctgtatttggggagggaaaacctt
tttaagcatggtggggcactcagataggagtgaatacacctacctggtg
DEFINITION Homo sapiens axin (AXIN1), mRNA.
ACCESSION XM_027520 SEQ ID NO:39
Gggcccggggtcccgccaccaccgcgcgcgggacagattgattcactttggagctgtaagtactgatgtattagggtgca
gcgctcattgttccttgacgcagagtcccaaaatgaatatccaagagcagggtttccccttggactcggagcaagtttcacc
gaagatgctccccgacccccagtgcctggtgaggagggagaactggtgtccacagacccgaggcccgccagctacagtt
tctgctccgggaaaggtgttggcattaaaggtgagacttcgacggccactccgaggcgctcggatctggacctggggtatg
agcctgagggcagtgcctcccccaccccaccatacttgaagtgggctgagtcactgcattccctgctggatgaccaagatg
ggataagcctgttcaggactttcctgaagcaggagggctgtgccgacttgctggacttctggtttgcctgcactggcttcagg
aagctggagccctgtgactcgaacgaggagaagaggctgaagctggcgagagccatctaccgaaagtacattcttgataa
caatggcatcgtgtcccggcagaccaagccagccaccaagagcttcataaagggctgcatcatgaagcagatgatcgatc
ctgccatgtttgaccaggcccagaccgaaatccaggccactatggaggaaaacacctatccctccttccttaagtctgatattt
atttggaatatacgaggacaggctcggagagccccaaagtctgtagtgaccagagctctgggtcagggacagggaaggg
catatctggatacctgccgaccttaaatgaagatgaggaatggaagtgtgaccaggacatggatgaggacgatggcagag
acgctgctccccccggaagactccctaagaagctgctcctggagacagctgccccgagggtctcctccagtagacggtac
agcgaaggcagagagttcaggtatggatcctggcgggagccagtcaacccctattatgtcaatgccggctatgccctggcc
ccagccaccagtgccaacgacagcgagcagcagagcctgtccagcgatgcagacaccctgtccctcacggacagcagc
gtggatgggatccccccatacaggatccgtaagcagcaccgcagggagatgcaggagagcgtgcaggtcaatgggcgg
gtgcccctacctcacattcccgtaagtaccgggtgccgaaggaggtccgcgtggagcctcagaagttcgcggaggagctc
atccaccgcctggaggctgtgcagcgcaagcgggaggccgaggagaagctggaggagcggctgaagcgcgtgcgcat
ggaggaggaaggtgaggacggcgatccatcgtcagggcccccagggccgtgtcacaagctgcctcccgcccccgcttg
gcaccacttcccgccccgctgtgtggacatgggctgtgccgggctccgggatgcacacgaggagaaccctgagagcatc
ctggacgagcacgtacagcgtgtgctgaggacacctggccgccagtcgcctgggcctggccatcgctccccggacagtg
ggcacgtggccaagatgccagtggcactggggggtgccgcctcggggcacgggaagcacgtacccaagtcaggggcg
aagctggacgcggccggcctgcaccccaccgacacgtccaccaccacgtccaccacagcacagcccggcccaagga
gcaggtggaggccgaggccacccgcagggcccagagcagcttcgcctggggcctggaaccacacagccatggggcaa
ggtcccgaggctactcagagagtgttggcgctgcccccaacgccagtgatggcctcgcccacagtgggaaggtgggcgt
ggcgtgcaaaagaaatgccaagaaggctgagtcggggaagagcgccagcaccgaggtgccaggtgcctcggaggatg
cggagaagaaccagaaaatcatgcagtggatcattgagggggaaaaggagatcagcaggcaccgcaggaccggccac
gggtcttcggggacgaggaagccacagccccatgagaactccagacccttgtcccttgagcacccctgggccggccctc
agctccggacctccgtgcagccctcccacctcttcatccaagaccccaccatgccaccccacccagctcccaaccccctaa
cccagctggaggaggcgcgccgacgtctggaggaggaagaaaagagagccagccgagcaccctccaagcagaggtat
gtgcaggaggtatgcggcggggacgcgcctgcgtcaggccagcgtgcgcgccggtgctgcacgtggtaccagccgtgt
cggacatggagctctccgagacagagacaagatcgcagaggaaggtgggcggcgggagtgcccagccgtgtgacagc
atcgttgtggcgtactacttctgcggggaacccatcccctaccgcaccctggtgaggggccgcgctgtcaccctgggccag
ttcaaggagctgctgaccaaaaagggcagctacagatactacttcaagaaagtgagcgacgagtttgactgtggggtggtg
tttgaggaggttcgagaggacgaggccgcctgcccgtctttgaggagaagatcatcggcaaagtggagaaggtggactg
ataggctggtgggctggccgctgtgccaggcgaggcccttggcgggcacgggtgtcacggccaggcagatgacctcgta
ctcaggagcccgatggggaacagtgttgggtgtaccacccatccctgtggtctacccgtgtctagaggcaggtagggggtc
cctccaagtggtccacaagcttctgtcctgcccccaaggaggcagcctggaccactcctcatagcaatacttggagggccc
agcccaagtgaggcagccgaggtccctgctgccagcttcaggtgacccccccccatcccccggcacctcccttgggcac
tgtgctgggatctactttccctctgggatttgcccacgtacccaggtctgggtggggcccaggcccggatgcagaggcct
gcagggcctctgtcaattgtacgcgccaccgagtgccttcaacacagcttgtctcttgcctgccactgtgtgaatcggcgac
ggagcactgcacctgcctccagccgccggctgtgcagtcctgggtcctcctttctgagggcccgtgtaaatatgtacatttct
caggctaggccagcaggggctgcccgagtctgtttttcatgcgatgacacttgtacaattaattatcttttcaaaggtacttgga
taataatgaaataaaactgtttttgaacctg
DEFINITION Homo sapiens AXIN2 (AXIN2) mRNA, complete cds.
ACCESSION AF205888 SEQ ID NO:40
Gttcagatgagcccctgctgacttgagagagacagagagaccacgccgattgctgagaggaactggaagaagaagagat
tccagactcagtgggaagagctccctcaccatgagtagcgctatgttggtgacttgcctcccggaccccagcagcagcttc
cggaggatgcccGgcggcccccagtgccaggggaagaaggggagaccccaccgtgtcaccatggtgggcaagggcc
aggtcaccaaacccatgtctgtctcttccaacaccaggcggaacgaagatgggttggggagccggaggggcgggcatct
ccggattcccctctgacccggtggaccaccttacactccttattgggcgatcaagacggtgcttacctgttccgaactttc
ctggagagggagaaatgcgtggataccttagacttctggtttgcctgcaatggattcaggcagatgaacctgaaggatacca
aactttacgagtagccaaagcgatctacaaaaggtacattgagaacaacagcattgtctccaagcagctgaagcctgcca
ccaagacctacataagagatggcatcaagaagcagcagattgattccatcatgtttgaccaggcgcagaccgagatccagt
cggtgatggaggaaaatgcctaccagatgtttttgacttctgatatatacctcgaatatgtgaggagtgggggagaaaacaca
gcttacatgagtaatgggggactcgggagcctaaaggtcgtgtgtggctatctccccaccttgaatgaagaagaggagtgg
acttgtgccgacttcaagtgcaaactttcgccaaacgtggttggcttgtccagcaaaactctgagggccacggcgagtgtga
ggtccacggaactgttgacagtggatacaggtccttcaagaggagcgatcctgttaatccttatcacataggttctggctatg
tctttgcaccagccaccagcgccaacgacagtgagatatccagtgatgcgctgacggatgattccatgtccatgacggaca
gcagtgtagatggaattcctccttatcgtgtgggcagtaagaaacagctccagagagaaatgcatcgcagtgtgaaggcca
atggcagagtgtctctacctcatttcccgagaacccaccgcctgcccaaggagatgacccccgtggaacccgccacctttg
cagctgagctgatctcgaggctggaaaagctgaagctggagttggagagccgccacagcctggaggagcgcctgcagc
agatccgagaggatgaagagagagagggctccgagctcacactcaattcgcgggagggggcgcacacgcagcacccc
ctctccctactgccctccggcagctacgaggaagacccgcagacgatactggacgatcacctgtccagggtcctcaagac
ccctggctgccagtctccgggcgtaggccgctatagccctcgctcccgctccccggaccaccaccaccaccaccattcgc
agtaccactccctgctcccgcccggtggcaagctgcctcccgcggccgcctcgccgggcgcctgccccctcctcggggg
caaaggctttgtgaccaagcagacgacgaagcatgtccaccaccactacatccaccaccatgccgtccccaagaccaagg
aggagatcgaggcggaggccacgcagcgggtgcactgcttctgccctgggggcagcgagtattactgatactcgaaatg
caaaagccactccaaggctccggaaaccatgcccagcgagcagtttggtgcccaaagcacaaaaaaggcctaccccttg
gagtctgcccgctcgtctccaggcgaacgagccagccggcaccatctgtgggggggcaacagcgggcacccccgcacc
accccccgtgcccacctgttcacccaggaccctgcgatgccttccctgaccccacccaacacgctggctcatctggagga
ggcctgtcgcaggctagctgaggtgtcgaagcccccaaagcagcggtgctgtgtggccagtcagcagagggacaggaat
cattcggccactgttcagacgggagccacacccttctccaatccaagcctggctccagaagatcacaaagagccaaagaa
actggcaggtgtccacgcgctccaggccagtgagttggttgtcacttactttttctgtggggaagaaattccataccggagga
tgctgaaggctcagagcttgaccctgggccactttaaagagcagctcagcaaaaagggaaattataggtattacttcaaaaa
agcaagcgatgagtttgcctgtggagcggtgtttgaggagatctgggaggatgagacggtgctcccgatgtatgaaggcc
ggattctgggcaaagtggagcggatcgattgagccctggggtctggctttggtgaactgttggagcccgaagctcttgtgaa
ctgtcttggctgtgagcaactgcgacaaaacattttgaaggaaaa
DEFINITION Homo sapiens frequently rearranged in advanced T-cell lymphomas
(FRAT1), mRNA.
ACCESSION XM_050913 SEQ ID NO:41
Attccggctcccgcggctgcaggcgcgcggctagagtgcctggcgggctccggcttccgcgtccgccccggccccggt
ccagacttagtcttcagctccgcgcccgctccgccgcggcccaccgcgcccgccggcagccgagcccccagcgacgcc
cgcacagctccgggtgcccagacagggggccatgccgtgccggagggaggaggaagaggaagccggcgaggaggc
ggagggggaggaagaggaggaggacagcttcctcctactgcagcagtcagtggcgctgggcagctcgggcgaggtgg
aacggctggtggcccagatcggcgagacgctgcagctggacgcggcgcagcacagcccggcctcgccgtgcgggccg
ccgggggcgccgctgcgggccccggggcccctggctgcggcggtgccggcggacaaggccaggtccccgacggtgc
cgctgctgctgccgcccgcgttggcggagactgtgggcccggcgccccctggggtcctgcgctgcgccctgggggacc
gcggccgcgtgcggggccgcgctgcgccctactgcgtggccgagctcgccacaggccccagcgcgctgtccccactgc
cccctcaggccgaccttgatgggcctccgggagctggcaagcagggcatcccgcagccgctgtcgggtccgtgccggc
gaggatggctccggggcgccgccgcctcccgccgcctgcagcagcgacgcgggtcccaaccagaaacccgcacaggc
gacgacgacccgcaccggcttctgcagcagctagtgctctctggaaacctcatcaaggaggccgtgcgaaggcttcattcg
cgacggctgcagttacgtgcaaagcttccccaacgcccgctcctgggacctctgtcggcccaggtgcatgaacccccttcg
cctcgcagccctcgcgcggcctgcagtgaccctggcgcctccgggagggcgcagctcagaactggcgacggcgttcttg
tgcctggcagctaacacgcccggggtggccacagcgccagcctcagactggagggcaaggggttcccttgagggctgc
agttctactcaggctggtggagaactctggcttttggaagcgagagtaaaaagctaatgacgaggaaccgaaaaatcgcga
gtgtttcgcgggtaactggggttgagggccaaaatatttggaatgaaggactttggccctatttaaggcagattttacagagc
gcacctcaaacgtacaagtcagtaggactccttatttggcgtgacccgacctggccgcggagcctgcatttcctcgcagcct
ctcagtgccctccagccccgcgaccatgtggccacaatccacgcttctccggatcgcggtgcgccggaaccacggaggat
gatgccagttacttgctttaccttttcagggctggctcctgatccactttgggggaggagaacatgagtagataatttcagggt
gcagcccaatctgccagacttaaaaaaaccatcttgtgtctttggaggtgctgcttaataccaaacatgcggtgccatgaagg
gaccctttgggggttgataggagttaacccctgcgctctctttgcaactgtctctcttctcagagtggtgggggaaggctgta
cgacacgggtggggaaaggaggtgggggcggggagtattgaatggtggtggaagggtagagaggcgcggagtgaacc
ccacgcccttctaaagtgtattttcagagccggcccgcctctcctcggttcaaggtcactgtttcctgggcacgcactgggtt
gcgggacagagtagccaggttctgccggtgctcggagaagagcgcagtgttttgcaagtgctggagtctcctgaggacac
gagcgtcgccgccaccgcgggtgtgggaaagcgcggacgtgctgggcggctgtgcttcggtaggcgaccaccgcccct
ggccgcgctccgggctttcacggaaactcccgagaccgggccctgggttcctcctctcctactcggctctgcagtcctactc
aagcgggtggctctgggatcctgggggcctgggttgggggctagggagacgccatgtgatggacactccagggacacac
agcctagcacagcagcttataatgggctctccggggccatttgcaataacagctgcaattccctggatagacgagttgatttc
ctccctctgcccctcccccagccatgccagctggcctttgtaagtgcaggaaaccgagtagaaaatgtgaccctccaaatg
gagaagctgcaggctttgccagaaccatggtgaagtgcttggaacatactgttcactcactctaaaggcgctgagactg
aagccggtgcagcagccttcagcttttggcagcatgaaagttagcagagacaaggacggcagcaaggtgacaacagtgg
tggcaactcctgggcagggtccagacaggccacaagaagtcagctatacagacactaaagtgattggaaatggatcatttg
gtgtggtatatcaagccaaactttgtgattcaggagaactggtcgccatcaagaaagtattgcaggacaagagatttaagaat
cgagagctccagatcatgagaaagctagatcactgacatagtccgattgcgatcttctactccagtggtgagaga
gatgaggtctatcttaatctggtgctggactatgttccggaaacagtatacagagttgccagacactatagtcgagccaaaca
gacgctccctgtgatttatgtcaagttgtatatgtatcagctgttccgaagtttagcctatatccattcctttggaatctgccatcg
ggatattaaaccgcagaacctcttgttggatcctgatactgctgtattaaaactctgtgactttggaagtgcaaagcagctggtc
cgaggagaacccaatgtttcgtatatctgttctcggtactatagggcaccagagttgatctttgagccactgattatacctcta
gtatagatgtatggtctgctggctgtgtgttggctgagatgttactaggacaaccaatatttccaggggatagtggtgtggatc
agttggtagaaataatcaaggtcctgggaactccaacaagggagcaaatcagagaaatgaacccaaactacacagaattta
aattccctcaaattaaggcacatccttggactaaggtcttccgaccccgaactccaccggaggcaattgcactgtgtagccgt
ctgctggagtatacaccaactgcccgactaacaccactggaagcttgtgcacattcattgatgaattaagggacccat
gtcaaactaccaaatgggcgagacacacctgcactcttcaacttcaccactcaagaactgtcaagtaatccacctctggctac
catccttattcctcctcatgctcggattcaagcagctgcttcaacccccacaaatgccacagcagcgtcag
DEFINITION Homo sapiens beta-catenin-interacting protein ICAT (ICAT), mRNA.
ACCESSION NM_020248 SEQ ID NO:43
Ggccgctcctgctgctgctactgccgccgccgcagcggctgctcgggctgagcacgccccggaacaggccgccgcgc
gctgcgcgccggacccgctgcccctgccggcccggccgggtcgggcggcccagggaccgacagacttgacaacggtg
acagcactggggcggcaccttcctacttctgcccagccacagccctcccctcacagttgagcacctgtttgcctgaagttaat
ttcaagaagcaggagtccccagagccaggcagggggatgaaccgcgagggagctcccgggaagagtccggaggagat
gtacattcagcagaaggtccgagtgctgctcatgctgcggaagatgggatcaaacctgacagccagcgaggaggagttcc
tgcgcacctatgcaggggtggtcaacagccagctcagccagctgcctccgcactccatcgaccagggtgcagaggacgt
gggatggcgttttccaggtcggagacggaagaccggaggcagtagctgcaaagcacttggaacaccctggatgctgttg
aagggccaagagatctgtgtggctcctgggccggctgaatggcagcagccccacttgcaccacctcccccttccctaccca
accctgccctgccccaccccacctcacagctactcagtggggctggcatcaagggagacaccagtggtgcgtttataa,ttg
gcttaaagggatggacttgtgattggctgcaggaagaaactttttatttatcttgaccaacagaccttttaatttttatttc
tgactcttattttttaaaaaattgcgcctcggtatctggcttccctggaagctctccgagctctggtgcttagttaggtcattttat
tagaaatgtgaagaggtctgattggctgcttaaactggaaagggactgtgattggctggttaatgggaaacggtttttttctttg
gctgcaggtgttctgctgatatcaacagcttccctattttgaatgcagaaaacagggtctgggacattagtcgttatatttgactt
gaaaagaaagaaaccaagtgcgcttgcaatattattacacaagacttgctgctgccttcacattgggagattgatt
ggctttcgatgcgtgtgtttggtttcccattggttcacctgtgactcctgttgccatggattcacccccctctgctgccggctctg
ggcctgagggtccacctggagagtacatttgctttaatgagtgcacctgcctccaccagcaaggggaccccgagaaccctg
agcagggtccacagctggaaagttgggcccctgaggagctttgtgtcgtcttgaacgagcagcccagggcctagaggtaa
ccgttaggcgggatttatgtgcactgcctgcatgagctggcaaccagccagcgtcccttggtgagaaagggattgctgagg
caccgtccaggccccaccggccaggccgcgcccagcagaggcgtactacccagctctgtcctcttggccatccttctgtgt
accacttcctgaggcctcattttgggggtcatcttggaaaggggaggagcttctcccagtgtgagaccccaaagactctgga
ggtcatctggcggaggtctctgggagcccagaacccacataaaagccccagcttggctcacaaggcccaggagacctcc
agctaaacaccaacccctgacctaccccagccaggctcctacctgtctgctgccagcacagtaggtcccggccagctctg
gatctctcatcggaggcccatgccctccactccactgcctttggaagggtctctctccaggtcagcctggaagggaaagt
atcgtttgtttatgaaatgccactgggacagctggctgggccttcaccaagcaagtcacttcagactggcccttaagccaaac
tcaggcccagaattgcagttcagaatggcagtcctggaggcagggggtgaggggcaggtctagtgttcctgcaccaaacc
taagtccttccacctgccacccccttccctgggagggaggtggtcctcctatctccctggctcactggcaggtgtgggatctg
gggagagcggctggagaaagatgcagtcctcaggaagggggccgccaccctcccctatgctgaatgctgaggccc
ctaggtgcccagggccagtgggaccctctcagaaccaaatctttcccctttctcggggcttggggctcgggccgtaggggc
tcctgagtgtcatgaagtgcacaggagccaaatgaccgagccctggagagccccatggtgggtaggtggttcgtgctgtgc
tctggcaccatcagcctgttccagaaggaggattcgagcatcaggctaagacctgtgtcctccaccatgcatcaccccta
gcctggttagctgacagtcagctgtggggaacacagctacaaacctaccctggcagggactgagagcatctcaggagg
ggcagcgcatggcatgtgctggagtgagcacacccgtgtgcacactcatacaatgtgcacacacacgcactctc
cccgctcaggggcctggaggtctggctgagcccctggggaaggtgagttctttcatctccctcctccatttcggagtggtt
ggagtcaggtacgaggccacattgctggctgccccctcttgtagctcctatagggcaacacctgaggatacctga
cttctgaagaggaaaagtcaaagtgttgctgtggctccatatttcaactaaaaatatatctgttggagaaagaaattaacaataa
agaattttcataggttaaaanaaaaag
DEFINITION Homo sapiens DvI-binding protein IDAX mRNA, complete cds.
ACCESSION AF272159 SEQ ID NO:44
Ggcggcaggaccagcatgcaccaccgaaagactcccagaggctggggaagctggctgcccgccagagccgtca
gcaaatggcaaatactcctctccaccttatcccctgaacatgcagaccaggcgggggatgcatgaacaagctca
aatgcggcgctgctgaagcagagatatgaatctccccgagcgcaggggactttgccgctatcccggctatagggggcat
ctcattacctccaggggtcatcgtcatgacagcccttcactcccccgcagcagcctcagcagccacacagacagtgcata
caattgccaatctggcagactgcccgcagaatcattcctcctcctcctcgtcctcctcagggggagctggcggagccaac
ccagccaagaagaagaggaaaaggtgtggggtctgcgtgccctgcaagaggctcatcaactgtggcgtctgcagcagttg
caggaaccgcaaaacgggacaccagatctgcaaatttagaaaatgtgaagagctaaagaaaaaacctggcacttcactag
agagaacacctgttcccagcgctgaagcattccgatggttcttttaaagcagtagtatatcttattttcaaggcatttggaaatg
aagggcaaactaatgtcttgttttaagaaactgcttagtccaccactgaagaaaatatccagaaattattttcattttatgtatagg
gatttcttcaaaaaaaaaaaaaaa
DEFINITION Homo sapiens orphan nuclear receptor (PAR1) mRNA, complete cds.
ACCESSION AF084645 SEQ ID NO:45
Cctctgaaggtctagaatcgatagtgaatcgtgggacgggaagaggaagcactgcctttcagtgggaaatctcggcc
tcagcctgcaagccaagtgttcacagtgaaaaaagcaagagaataagctaatactcctgtcctgaacaaggcagcggctcc
ttggtaaagctactccttgatcgatcctttgcaccggattgttcaaagtggaccccaggggagaagtcggagcaaagaactt
accaccaagcagtccaagaggcccagaagcaaacctggaggtgagacccaaagaaagctggaaccatgctgactttgta
cactgtgaggacacagagtctgttcctggaaagcccagtgtcaaacgcagatgaggaagtcggaggtccccaaatctgccg
tgtatgtggggacaaggccactggctatcacttcaatgtcatgacatgtgaaggatgcaagggctttttcaggagggccatg
aacgcaacgcccggctgaggtgccccuccggaagggcgcctgcgagatcacccggagacccggcgacagagcca
ggcctgccgcctgcgcaagtgcctggagagcggcatgaagaaggagatgatcatgtccgacgaggccaggaggagag
gcgggccttgatcaagcggaagaaaagtgaacggacagggactcagccactgggaagcaggggctgacagaggagc
agcggatgatgatcagggagctgatggacgctcagatgaaaacctttgacactaccttctcccatttcaagaatttccggctg
ccaggggtgcttagcagggctgcgagttgccagagtctctgcaggccccatcgagggaagaagctgccaagtggagcc
aggtccggaaagatctgtgctctttgaaggtctctctgcagctgcggggggaggatggcactggacacacccc
cagccgacagtggcgggaaagagatcttctccctgctgccccacatggctgacatgtcaacctacatgttcaaaggcatcat
cagctttgccaaagtcatctcctacttcagggacttgcccatcgaggaccagatctccctgctgaagggggccgcacgag
ctcaactgagaacaacacagttcaacgcggagactggaacctgggagaggccggctacctactgcuggag
acactgcagaggcttccagcaacttctactggagcccatgctgaccaaacatgctgaagaagctgcagctgcatga
ggaggagtatagctgatgcaggccatctccctcttctccccagaccgcccagaagctgcagcaccgcaggtggacca
gctgcaggagcaattcgccattactctgaagtcctacattgaatgcaatcggccccagcctgctcataggttcttgttcctgaa
gatcatggctatgctcaccgagctccgcagcatcaatgctcagcacacccagcggctgctgcgcatccaggacatacacc
cctttgctacgcccctcatgcaggagttgttcggcatcacaggtagctgagcggctgcccttgggtgacacctccgagagg
cagccagacccagagccctctgagccgccactcccgggccaagacagatggacactgccagagccgacaatgccctg
ctggcctgtctccctagggattcctgcatgacagctggctagcatactcaggaggacatgggagccccccaccccca
gttcagtctgtagggagtgaagccacagactcttacgtggagagtgcactgacctgtaggtcaggaccatcagagaggcaa
ggttgccctttccttttaaaaggccctgtggtctggggagaaatccctcagatcccactaaagtgtcaaggtgtggaagggac
caagcgaccaaggataggccatctggggtctatgcccacatacccacgtttgttcgcttcctgagtcttttcattgctacctcta
atagtcctgtctcccacttcccactcgttcccctcctcttccgagctgctttgtgggctcaaggcctgtactcatcggcaggtgc
atgagtatctggggagtcctctagagagatgagaagccaggaggcctgcaccaaatgtcagaagcttggcatgacctcatt
ccggccacatcattctgtgtctctgcatccatttgaacacattattaagcactgataataggtagcctgctgtggggtatacagc
attgactcagatatagatcctgagctcacagagtttatagttaaaaaaacaaacagaaacacaaacaatttggatcaaaagga
gaaaatgataagtgacaaaagcagcacaaggaatttccctgtgtggatgctgagctgtgatggcaggcactgggtacccaa
gtgaaggttcccgaggacatgagtctgtaggagcaagggcacaaactgcagctgtgagtgcgtgtgtgtgatttggtgtag
gtaggtctgtttgccacttgatggggcctgggtttgttcctggggctggaatgctgggtatgctctgtgacaaggctacgctga
caatcagttaaacacaccggagaagaaccatttacatgcaccttatatttctgtgtacacatctattctcaaagctaaagggtat
gaaagtgcctgccttgtttatagccacttgtgagtaaaaatttttttgcattttcacaaattatactttatataaggcattccacacct
aagaactagttttgggaaatgtagccctgggtttaatgtcaaatcaaggcaaaaggaattaaataatgtacttttggctaaaaa
aaaaaaaaaaaaaaaaaaaaaaaaaaaaaaaaaaaaaaaaaaa
DEFINITION Homo sapiens transforming growth factor beta-activated
kinase-binding protein 1 (TAB1), mRNA.
ACCESSION XM_010000 SEQ ID NO:46
Cccgcaggggttcctccaagatggcggcgcagaggaggagcttgctgcagagtgagcagcagccaagctggacagat
gacctgcctctctgccacctctctggggttggctcagcctccaaccgcagctactctgctgatggcaagggcactgagagc
cacccgccagaggacagctggctcaagttcaggagtgagaacactgcttcctgtatggggtcttcaacggctatgatggc
aaccgagtgaccaacttcgtggcccagcggctgtccgcagagctcctgctgggccagctgaatgccgagcacgccgagg
ccgatgtgcggcgctgctgcaggccttcgatgtggtggagaggagcttcctggagtccattgacgacgccttggctg
agaaggcaagcctccagtcgcaattgccagagggagtccctcagcaccagctgcctcctcagtatcagaagatccttgaga
gactcaagacgttagagagggaatcgggaggggccatggccgttgtggcggtccttctcaacaacaagctctacgtcg
ccaatgtcggacaaccgtgcacttttatgcaaatcgacagtggatgggttgcaggtgacacagctgaacgtggaccaca
ccacagagaacgaggatgagctcttccgtctttcgcagctgggcttggatgctggaaagatcaagcaggtggggatcatct
gtgggcaggagagcacccggcggatcggggattacaaggttaaatatggctacacggacattgaccttctcagcgctgcc
aagtccaaaccaatcatcgcagagccagaaatccatggggcacagccgctggatggggtgacgggcttcttggtgctgat
gtcggaggggttgtacaaggccctagaggcagcccatgggcctgggcaggccaaccaggagattgctgcgatgattgac
actgagtttgccaagcagacctccctggaagcagtggcccaggccgtcgtggaccgggtgaagcgcatccacagcgaca
ccttcgccagtggtggggagcgtgccaggttctgcccccggcacgaggacatgaccctgctagtgaggaactttggctac
ccgctgggcgaaatgagccagcccacaccgagcccagccccagctgcaggaggacgagtgtaccctgtgtctgtgccat
actccagcgcccagagcaccagcaagaccagcgtgaccctctcccttgtcatgccctcccagggccagatggtcaacgg
ggctcacagtgcttccaccctggacgaagccacccccaccctcaccaaccaaagcccgaccttaaccctgcagtccacca
acacgcacacgcagagcagcagctccagctctgacggaggcctcttccgctcccggcccgcccactcgctcccgcctgg
cgaggacggtcgtgttgagccctatgtggactttgctgagttttaccgcctctggagcgtggaccatggcgagcagagcgtg
gtgacagcaccgtagggcagccggagaatgcagcccaagcagggcctggcatggggcaggacagggtccagccttttc
ctaacatctgcctgtgccacaacggccagcaggtgccccatcctctgcccacagcagactctgtcccatggctctccgggc
agtagagtgtgtgagtgcagactggacctgtggttcataccttgtcaccacccgggaagctgaaggccacttcctcccagat
ggcctcagccaggaccatcgccctttctcagagcagagggccaggtatagaaaccgcagtgggcctgcaagccgcccga
gcctccccagcagcctcctacagagcaggaagaggcgccctgtgaaccctgagtgttgcaggcccagcagaccctgctg
tcccaagcccacccctcctcccaccatcacctccctcacctcgggacagtagccctccacttctccagcctctcagccctgt
gctcctgcatccagagtggaacccaggctggtgtccgcatctgtccctgggccccacccctggacctgccttggttgtgtca
tctgttgtaaacgttcaggaggaccagggcagcatctggggcctgggatggccacagaaggggcaggccaggtggaaa
ggagccagggggaagtggtctaagagacctggaactgccagaggatggcggcctgggcttccccagagccaggcgtgc
gggagaggtgaggactggccccggtgggctgaggcaggggccgctgtcgtcaggcctgagccagggtgagctggtgc
ctgccttgcattttccttctggtgctgtgaagaccataggctggcaggcagctgagatgaactgtctttaccactgatgagggg
cctctgccggctgagggtagcaagcaggggttgtgagtcaggctgggggacttgtttgaaagaaagaggagtggaaaaatg
gttccaggagggaagaggttctttgagacacagtaccctgggaggcataggagaagggtcgggccagcccagcccagg
gcctgagttagactatttcccacatgttctctgccttcagtggggagggggtgccacaagggctgtcggccaggattgccac
tcctgtttcagaggaagcaggccgagagacttgcaccttggccaagccacaaaatcagtggggcagccagagctcagac
ctgagccattttgtcagtatccaggaccccacggattctccacgccctccccatctcccagtctccctgccccccatgcccca
gaccggcccaccagggactagccgctgtcgcacagcctctggggtgcttggtctctgcgatcaggcctgacagctc
tgtggcctgggaatccattttcctgcggcagagcagggcctggtgtggaaccagggagctgtgggaagccacagcagaa
atggaagaaaaacaggtctcagcccagggtcctcgctcactccctcactccccatgaagccatctctgttctgcagaga
gaggatttaaagtcagtcacaaaggcttgggaacaaaaggaattc
Nuclear actin components:
DEFINITION H.sapiens mRNA for beta-catenin.
ACCESSION X87838 SEQ ID NO:47
Aagcctctcggtctgtggcagcagcgttggcccggccccgggagcggagagcgaggggaggcggagacggaggaag
gtctgaggagcagcttcagtccccgccgagccgccaccgcaggtcgaggacggtcggactcccgcggcgggaggagc
ctgttcccctgagggtatttgaagtataccatacaactgttttgaaaatccagcgtggacaatggctactcaagctgatttgatg
gagttggacatggccatggaaccagacagaaaagcggctgttagtcactggcagcaacagtcttacctggactctggaatc
cattctggtgccactaccacagctccttctctgagtggtaaggcaatcctgaggaagaggatgtggatacctcccaagtcct
gtatgagtgggaacagggattttctcagtccttcactcaagaacaagtagctgatattgatggacagtatgcaatgactcgag
ctcagagggtacgagctgctatgttccctgagacattagatgagggcatgcagatcccatctacaaagtttgatgctgctcat
cccactaatgtccagcgtttggctgaaccatcacagatgctgaaacatgcagttgtaaacttgattaactatcaagatgatgca
gaacttgccacacgtgcaatccctgaactgacaaactgctaatgacgaggaccaggtggtggttaataaggctgcagtt
atggtccatcagctttctaaaaaggaagcttccagacacgctatcatgcgttctcctcagatggtgtctgctattgtacgtacca
tgcagaatacaaatgatgtagaaacagctcgttgtaccgctgggaccttgcataacctttcccatcatcgtgagggcttactg
gccatctttaagtctggaggcattcctgccctggtgaaaatgcttggttcaccagtggattctgtgttgttttatgccattacaact
ctccacaaccttttattacatcaagaaggagctaaaatggcagtgcgtttagctggtgggctgcagaaaatggttgccttgctc
aacaaaacaaatgttaaattcttggctattacgacagactgccttcaaattttagcttatggcaaccaagaaagcaagctcatc
atactggctagtggtggaccccaagctttagtaaatataatgaggacctatacttacgaaaaactactgtggaccacaagcag
agtgctgaaggtgctatctgtctgctctagtaataagccggctattgtagaagctggtggaatgcaagctttaggacttcacct
gacagatccaagtcaacgtcttgttcagaaatgtctttggactctcaggaatctttcagatgctgcaaataaacaggaagggat
ggaaggtctccttgggactcttgttcagcttctgggttcagatgatataaatgtggtcacctgtgcagctggaattctttctacc
tcacttgcaataattataagaacaagatgatggtctgccaagtgggtggtatagaggctcttgtgcgtactgtccttcgggctg
gtgacagggaagacatcactgagcctgccatctgtgctcttcgtcatctgaccagccgacaccaagaagcagagatggcc
cagaatgcagttcgccttcactatggactaccagttgtggttaagctcttacacccaccatcacactggcctctgataaaggct
actgttggattgattcgaaatcttgccctcccgcaaatcatgcacctttgcgtgagaagggtgccattccacgactagttc
agttgcttgttcgtgcacatcaggatacccagcgccgtacgtccatgggtgggacacagcagcaatttgtggagggggtcc
gcatggaagaaatagttgaaggttgtaccggagcccttcacatcctagctcgggatgttcacaaccgaattgttatcagagg
actaaataccattccattgtttgtgcagctgctttattctcccattgaaaacatccaaagagtagctgcaggggtcctctgtgaa
cttgctcaggacaaggaagctgcagaagctattgaagctgagggagccacagctcctctgacagagttacttcactctagg
aatgaaggtgtggcgacatatgcagctgctgttttgttccgaatgtctgaggacaagccacaagattacaagaaacggctttc
agttgagctgaccagctctctcttcagaacagagccaatggcttggaatgagactgctgatcttggacttgatattggtgccca
gggagaaccccttggatatcgccaggatgatcctagctatcgttcttttcactctggtggatatggccaggatgccttgggtat
ggaccccatgatggaacatgagatgggtggccaccaccctggtgctgactatccagttgatgggctgccagatctggggc
atgcccaggacctcatggatgggctgcctccaggtgacagcaataagatggcctggtttgatactgacctgtaaatcatcctt
tagctgtattgtctgaacttgcattgtgattggcctgtagagttgctgagagggctcgaggggtgggctgaatctcagaaat
gcctgacacactaaccaagctgagtttcctatgggaacaattgaagtaaactttttgttctggtccttaggtcgaggaaaaac
aatacaaatggattttgggagtgactcaagaagtgaagaatgcacaagaatggatcacaagatggaatttagcaaaccctag
ccttgcttgttaaaatttttttttttttttaagaatatctgtaatggtactgactttgcttgctttgaagtagctctttttttttttttt
ttttttttgcagtaactgttttttaagtctctcgtagtgttaagttatagtgaatactgctacagcaatttctaatttttaagaattgagt
aatggtgtagaacactaaattcataatcactctaattaattgtaatctgaaagtgtaacaattgtgtagcctttttgtataa
aatagacaaatagaaaatggtccaattagtttcctttttaatatgcttaaaataagcaggtggatctatttcatgtttttgatcaaa
actatttgggatatgtatgggtagggtaaatcagtaagaggtgttattggaaacttgttttggacagtttaccagttgccttttat
cccaaagttgaacctgctgtgatacgatgcttcaagagaaaatgcggttataaaaaatggttcagaattaaaacttttaattc
att
DEFINITION Homo sapiens b-TRCP variant E3RS-IkappaB mRNA, partial cds.
ACCESSION AF101784 SEQ ID NO:48
Atggacccggccgaggcggtgctgcaagagaaggcactcaagtttatgtgctctatgcccaggtctctgtggctgggctg
ctccagcctggcggacagcatgccttcgctgcgatgcctgtataacccagggactggcgcactcacagctttccagaattcc
tcagagagagaagacttaatggcgaaccccctaggaagataataccagagaagaattcacttagacagacatacaa
cagctgtgccagactctgcttaaaccaagaaacagtatgtttagcaagcactgctatgaagactgagaattgtgtggccaaa
acaaaacttgccaatggcacttccagtatgattgtgcccaagcaacggaaactctcagcaagctatgaaaaggaaaaggaa
ctggtcaaatactttgagcagtggtcagagtcagatcaagtggaatttgtggaacatcttatatcccaaatgtgtcattacc
aacatgggcacataaactcgtatcttaaacctatgttgcagagagatttcataactgctctgccagctcggggattggatcata
tcgctgagaacattctgtcatacctggatgccaaatcactatgtgctgctgaacttgtgtgcaaggaatggtaccgagtgacct
ctgatggcatgctgtggaagaagcttatcgagagaatggtcaggacagattctctgtggagaggcctggcagaacgaaga
ggatggggacagtatttattcaaaaacaaacctcctgacgggaatgctcctccaaactctttttatagagcactttatcctaaaa
ttatacaagacattgagacaatagaatctaattggagatgtggaagacatagtttacagagaattcactgccgaagtgaaaca
agcaaaggagtttactgtttacagtatgatgatcagaaaatagtaagcggccttcgagacaacacaatcaagatctgggata
aaaacacattggaatgcaagcgaattctcacaggccatacaggttcagtcctctgtatccagtatgatgagagagtgatcata
acaggatcatcggattccacggtcagagtgtgggatgtaaatacaggtgaaatgctaaacacgttgattcaccattgtgaag
cagttctgcacttgcgtttcaataatggcatgatggtgacctgctccaaagatcgttccattgctgtatgggatatggcctcccc
aactgacattaccctccggagggtgctggtcggacaccgagctgctgtcaatgttgtagactttgatgacaagtacattgtttc
tgcatctggggatagaactataaaggtatggaacacaagtacttgtgaatttgtaaggaccttaaatggacacaacgaggc
attgcctgtttgcagtacagggacaggctggtagtgagtggctcatctgacaacactatcagattatgggacatagaatgtgg
tgcatgtttacgagtgttagaaggccatgaggaattggtgcgttgtattcgatttgataacaagaggatagtcagtggggccta
tgatggaaaaattaaagtgtgggatcttgtggctgctttggacccccgtgctcctgcagggacactctgtctacggacccttgt
ggagcattccggaagagtttttcgactacagtttgatgaattccagattgtcagtagttcacatgatgacacaatcctcatctgg
gacttcctaaatgatccagctgcccaagctgaacccdcccgttccccttctcgaacatacacctacatctccagataa
DEFINiTION Human CREB-binding protein (CBP) mRNA, complete cds.
ACCESSION 1347741 SEQ ID NO:49
Tgaggaatcaacagccgccatcttgtcgcggacccgaccggggcttcgagcgcgatctactcggccccgccggtcccg
ggccccacaaccgcccgcgctcgctcctctccctcgcagccggcagggcccccgacccccgtccgggccctcgccggc
ccggccgcccgtgcccggggctgttttcgcgagcaggtgaaaatggctgagaacttgctggacggaccgcccaacccca
aaagagccaaactcagctcgcccggtttctcggcgaatgacagcacagattttggatcattgtttgacttggaaaatgatcttc
ctgatgagctgatacccaatggaggagaattaggccttttaaacagtgggaaccttgttccagatgctgcttccaaacataaa
caactgtcggagcttctacgaggaggcagcggctctagtatcaacccaggaataggaaatgtgagcgccagcagccccgt
gcagcagggcctgggtggccaggctcaagggcagccgaacagtgctaacatggccagcctcagtgccatgggcaagag
ccctctgagccagggagattcttcagcccccagcctgcctaaacaggcagccagcacctctgggcccacccccgctgcct
cccaagcactgaatccgcaagcacaaaagcaagtggggctggcgactagcagccctgccacgtcacagactggacctg
gtatctgcatgaatgctaactttaaccagacccacccaggcctcctcaatagtaactctggccatagcttaattaatcaggcttc
acaagggcaggcgcaagtcatgaatggatctcttggggctgctggcagaggaaggggagctggaatgccgtaccctact
ccagccatgcagggcgcctcgagcagcgtgctggctgagaccctaacgcaggtttccccgcaaatgactggtcacgcgg
gactgaacaccgcacaggcaggaggcatggccaagatgggaataactgggaacacaagtccatttggacagccctttagt
caagctggagggcagccaatgggagccactggagtgaacccccagttagccagcaaacagagcatggtcaacagtttgc
ccaccttccctacagatatcaagatacttcagtcaccaacgtgccaaatatgtctcagatgcaaacatcagtgggaattgta
cccacacaagcaattgcaacaggcaccactgcagatcctgaaaaacgcaaactgatacagcagcagctggttctactgctt
catgctcataagtgtcagagacgagagcaagcaaacggagaggttcgggcctgctcgctcccgcattgtcgaaccatgaa
aaacgttttgaatcacatgacgcattgtcaggctgggaaagcctgccaagttgcccattgtgcatcttcacgacaaatcatctc
tcattggaagaactgcacacgacatgactgtcctgtttgcctccctttgaaaaatgccagtgacaagcgaaaccaacaaacc
atcctggggtctccagctagtggaattcaaaacacaattggttctgttggcacagggcaacagaatgccacttctttaagtaac
ccaaatcccatagaccccagctccatgcagcgagcctatgctgctctcggactcccctacatgaaccagccccagacgca
gctgcagcctcaggttcctggccagcaaccagcacagcctcaaacccaccagcagatgaggactctcaaccccctggga
aataatccaatgaacattccagcaggaggaataacaacagatcagcagcccccaaacttgatttcagaatcagctcttccga
cttccctgggggccacaaacccactgatgaacgatggctccaactctggtaacattggaaccctcagcactataccaacag
cagctcctccttctagcaccggaggaaaggctggcacgacatgtcactcaggacctgcggagccatctagtgcata
aactcgtccaagccatcttcccaacacctgatcccgcagctctaaaggatcgccgcatggaaaacctggtagcctatgctaa
gaaagtggaaggggacatgtacgagtctgccaacagcagggatgaatattatcacttattagcagagaaaatctacaagat
acaaaaagaactagaagaaaaacggaggtcgcgtttacataaacaaggcatcttggggaaccagccagccttaccagccc
cgggggctcagccccctgtgattccacaggcacaacctgtgagacctccaaatggacccctgtccctgccagtgaatcgca
tgcaagtttctcaagggatgaattcatttaaccccatgtccttggggaacgtccagttgccacaagcacccatgggacctcgt
gcagcctccccaatgaaccactctgtccagatgaacagcatgggctcagtgccagggatggccatttctccttcccgaatgc
ctcagcctccgaacatgatgggtgcacacaccaacaacatgatggccaaggcgcccgctcagagccagtttctgccacag
aaccagttcccgtcatccagcggggcgatgagtgtgggcatggggcagccgccagcccaaacaggcgtgtcacaggga
caggtgcctggtgctgctcttcctaaccctctcaacatgctggggcctcaggccagccagctaccttgccctccagtgacac
agtcaccactgcacccaacaccgcctcctgcttccacggctgctggcatgccatctctccagcacacgacaccacctggga
tgactcctccccagccagcagctcccactcagccatcaactcctgtgtcgtcttccgggcagactcccaccccgactcctgg
ctcagtgcccagtgctacccaaacccagagcacccctacagtccaggcagcagcccaggcccaggtgaccccgcagcct
caaaccccagttcagcccccgtctgtggctacccctcagtcatcgcagcaacagccgacgcctgtgcacgcccagcctcct
ggcacaccgctttcccaggcagcagccagcattgataacagagtccctaccccctcctcggtggccagcgcagaaaccaa
ttcccagcagccaggacctgacgtacctgtgctggaaatgaagacggagacccaagcagaggacactgagcccgatcct
ggtgaatccaaaggggagcccaggtctgagatgatggaggaggatttgcaaggagcttcccaagttaaagaagaaacag
acatagcagagcagaaatcagaaccaatggaagtggatgaaaagaaacctgaagtgaaagtagaagttaaagaggaaga
agagagtagcagtaacggcacagcctctcagtcaacatctccttcgcagccgcgcaaaaaaatctttaaaccagaggagtt
acgccaggccctcatgccaaccctagaagcactgtatcgacaggacccagagtcattacctttccggcagcctgtagatcc
ccagctcctcggaattccagactattttgacatcgtaaagaatcccatggacctctccaccatcaagcggaagctggacaca
gggcaataccaagagccctggcagtacgtggacgacgtctggctcatgttcaacaatgcctggctctataatcgcaagaca
tcccgagtctataagttttgcagtaagcttgcagaggtctttgagcaggaaattgaccctgtcatgcagtcccttggatattgct
gtggacgcaagtatgagttttccccacagactttgtgctgctatgggaagcagctgtgtaacattcctcgcgatgctgcctact
acagctatcagaataggtatcatttctgtgagaagtgtttcacagagatccagggcgagaatgtgaccctgggtgacgaccc
ttcacagccccagacgacaatttcaaaggatcagtttgaaaagaagaaaaatgataccttagaccccgaacctttcgttgatt
gcaaggagtgtggccggaagatgcatcagatttgcgttctgcactatgacatcatttggccttcaggttttgtgtgcgacaact
gcttgaagaaaactggcagacctcgaaaagaaaacaaattcagtgctaagaggctgcagaccacaagactgggaaacca
cttggaagaccgagtgaacaaatttttgcggcgccagaatcaccctgaagccggggaggtttttgtccgagtggtggccag
ctcagacaagacggtggaggtcaagcccgggatgaagtcacggtttgtggattctggggaaatgtctgaatctttcccatat
cgaaccaaagctatgtttgcttttgaggaaattgacggcgtggatgtctgcttttttggaatgcacgtccaagaatacggctctg
attgcccccctccaaacacgaggcgtgtgtacatttcttatctggatagtattcatttcttccggccacgttgcctccgcacagc
cgtttaccatgagatccttattggatatttagagtatgtgaagaaattagggtatgtgacagggcacatctgggcctgtcctcca
agtgaaggagatgattacatcttccattgccacccacctgatcaaaaaatacccaagccaaaacgactgcaggagtggtac
aaaaagatgctggacaaggcgtttgcagagcggatcatccatgactacaaggatattttcaaacaagcaactgaagacagg
ctcaccagtgccaaggaactgccctattttgaaggtgatttctggcccaatgtgttagaagagagcattaaggaactagaaca
agaagaagaggagaggaaaaaggaagagagcactgcagccagtgaaaacactgagggcagtcagggcgacagcaag
aatgccaagaagaagaacaacaagaaaaccaacaagaacaaaagcagcatcagccgcgccaacaagaagaagcccag
catgcccaacgtgtccaatgacctgtcccagaagctgtatgccaccatggagaagcacaaggaggtcttcttcgtgatccac
ctgcacgctgggcctgtcatcaacaccctgcccaccatcgtcgaccccgacccactgctcagctgtgacatcatggatggg
cgcgacgccttcctcaccctcgccagagacaagcactgggagttctcctccttgcgccgctccaagtggtccacgctctgc
atgctggtggagctgcacacccagggccaggaccgctttgtctacacctgcaacgagtgcaagcaccacgtggagacgc
gctggcactgcactgtgtgcgaggactacgacctctgcatcaactgctataacacgaagagccatgcccataagatggtga
agtgggggctgggcctggatgacgagggcagcagccagggcgagccacagtcaaagagcccccaggagtcacgccg
ggtgagcatccagcgctgcatccagtcgctggtgcacgcgtgccagtgccgcaacgccaactgctcgctgccatcctgcc
agaagatgaagcgggtggtgcagcacaccaagggctgcaaacgcaagaccaacgggggctgcccggtgtgcaagcag
ctcatcgccctctgctgctaccacgccaagcactgccaagaaaacaaatgccccgtgcccttctgcctcaacatcaaacaca
agctccgccagcagcagatccagcaccgcctgcagcaggcccagctcatgcgccggcggatggccaccatgaacaccc
gcaacgtgcctcagcagagtctgccttctcctacctcagcaccgcccgggacccccacacagcagcccagcacacccca
gacgccgcagccccctgcccagccccaaccctcacccgtgagcatgtcaccagctggcttccccagcgtggcccggact
cagccccccaccacggtgtccacagggaagcctaccagccaggtgccggcccccccacccccggcccagccccctcct
gcagcggtggaagcggctcggcagatcgagcgtgaggcccagcagcagcagcacctgtaccgggtgaacatcaacaac
agcatgcccccaggacgcacgggcatggggaccccggggagccagatggcccccgtgagcctgaatgtgccccgacc
caaccaggtgagcgggcccgtcatgcccagcatgcctcccgggcagtggcagcaggcgccccttccccagcagcagcc
catgccaggcttgcccaggcctgtgatatccatgcaggcccaggcggccgtggctgggccccggatgcccagcgtgcag
ccacccaggagcatctcacccagcgctctgcaagaactgctgcggaccctgaagtcgcccagctcccctcagcagcaac
agcaggtgctgaacattctcaaatcaaacccgcagctaatggcagctttcatcaaacagcgcacagccaagtacgtggcca
atcagcccggcatgcagccccagcctggcctccagtcccagcccggcatgcaaccccagcctggcatgcaccagcagc
ccagcctgcagaaactgaatgacatgcaggctggcgtgccgcggcccggtgtgcctccacagcagcaggcgatgggag
gcctgaacccccagggccaggccttgaacatcatgaacccaggacacaaccccaaaatggcgagtatgaatccacagta
ccgagaaatgttacggaggcagctgctgcagcagcagcagcaacagcagcagcaacaacagcagcaacagcagcagc
agcaagggagtgccggcatggctgggggcatggcggggcacggccagttccagcagcctcaaggacccggaggctac
ccaccggccatgcagcagcagcagcgcatgcagcagcatctccccctccagggcagctccatgggccagatggcggct
cagatgggacagcttggccagatggggcagccggggctgggggcagacagcacccccaacatccagcaagccctgca
gcagcggattctgcagcaacagcagatgaagcagcagattgggtccccaggcaagccgaaccccatgagcccccagca
acacatgctctcaggacagccacaggcctcgcatctccctggccagcagatcgccacgtcccttagtaaccaggtgcggtc
tccagcccctgtccagtctacacggccccagtcccagcctccacattccagcccgtcaccacggatacagccccagccttc
gccacaccacgtctcaccccagactggttccccccaccccggactcgcagtcaccatggccagctccatagatcagggac
acttggggaaccccgaacagagtgcaatgctcccccagctgaacaccaccagcaggagtgcgctgtccagcgaactgtc
cctggtcggggacaccacgggggacacgctagagaagtttgtggagggcttgtagcattgtgagagcatcaccttttccctt
tcatgttcttggaccttttgtactgaaaatccaggcatctaggttctttttattcctagatggaactgcgacttccgagccatggaa
gggtggattgatgtttaaagaaacaatacaaagaatatatttttttgttaaaaaccagttgatttaaatatctggtctctctctttggt
ttttttttggcgggggggtggggggggttcttttttttccgttttgtttttgtttggggggaggggggttttgtttggattctttttgtc
gtcattgctggtgactcatgcctttttttaacgggaaaaacaagttcattatattcatattttttatttgtattttcaagactttaaacat
ttatgtttaaaagtaagaagaa2aataatattcagaactgattcctgaaataatgcaagattataatgtatcccgataactttgtg
atgtttcgggaagatttttttctatagtgaactctgtgggcgtctcccagtattaccctggatgataggaattgactccggcgtgc
acacacgtacacacccacacacatctatctatacataatggctgaagccaaacttgtcttgcagatgtagaaattgttgctttgt
ttctctgataaaactggttttagacaaaaaatagggatgatcactcttagaccatgctaatgttactagagaagaagccttctttt
ctttcttctatgtgaaacttgaaatgaggaaaagcaattctagtgtaaatcatgcaagcgctctaattcctataaatacgaaactc
gagaagattcaatcactgtatagaatggtaaaataccaactcatttcttatatcatattgttaaataaactgtgtgcaacagacaa
aaagggtggtccttcttgaattcatgtacatggtattaacaattagtgttcggggttttttgttatgaaaatgctgttttcaacattgt
atttggactatgcatgtgttttttccccattgtatataaagtaccgcttaaaattgatataaattactgaggtttttaacatgtattctg
ttctttaagatcccctgtaagaatgtttaaggtttttatttatttatatatattttttggtctgttctttgtaaaaaaaaaaaaa
DEFINITION Homo sapiens C-terminal binding protein 1 CTBP1, mRNA.
ACCESSION NM_001328 SEQ ID NO:50
Cgcgcagcgcgccggagtggtcggggcccgcggccgctcgcgcctctcgatgggcagctcgcacttgctcaacaagg
gcctgccgcttggcgtccgacctacgatcatgaacgggcccctgcacccgcggcccctggtggcattgctggatggccgg
gactgcacagtggagatgcccatcctgaaggacgtggccactgtggccttctgcgacgcgcagtccacgcaggagatcca
tgagaaggtcctgaacgaggctgtgggggccctgatgtaccacaccatcactctcaccagggaggacctggagaagttca
aagccctccgcatcatcgtccggattggcagtggttttgacaacatcgacatcaagtcggccggggatttaggcattgccgt
ctgcaacgtgcccgcggcgtctgtggaggagacggccgactcgacgctgtgccacatcctgaacctgtaccggcgggcc
acctggctgcaccaggcgatgcgggagggcacacgagtccagagcgtcgagcagatccgcgaggtggcgtccggcgc
tgccaggatccgcggggagaccttgggcatcatcggacttggtcgcgtggggcaggcagtggcgctgcgggccaaggc
cttcggcttcaacgtgctctcacgacccttacttgtcggatggcgtggagcgggcgctggggctgcagcgtgtcagcacc
ctgcaggacctgctcttccacagcgactgcgtgaccctgcactgcggcctcaacgagcacaaccaccacctcatcaacga
cttcaacgtcaagcagatgagacaaggggccttcctggtgaacacagcccggggtggcctggtggatgagaaggcgctg
gcccaggccctgaaggagggccggatccgcggcgcggccctggatgtgcacgagtcggaacccttcagctttagccag
ggacctctgaaggatgcacccaacctcatctgcaccccccatgctgcatggtacagcgagcaggcatccatcgagatgcg
agaggaggcggcacgggagatccgcagagccatcacaggccggatcccagacagcctgaagaactgtgtcaacaagg
accatctgacagccgccacccactgggccagcatggaccccgccgtcgtgcaccctgagctcaatggggctgcctatagg
taccctccgggcgtggtgggcgtggcccccactggcatcccagctgctgtggaaggtatcgtccccagcgccatgtccctg
tcccacggcctgccccctgtggcccacccgccccacgccccttctcctggccaaaccgtcaagcccgaggcggatagag
accacgccagtgaccagttgtagcccgggaggagctctccagcctcggcgcctggggcagcgggcccggaaaccctcg
accagagtgtgtgagagcatgtgtgtggtggcccctggcactgcagagactggtccgggctgtcaggagggcgggaggg
cgcagcgctgggcctcgtgtcgcttgtcgtccgtcctgtgggcgctctgccctgtgtccttcgcgttcatcgttaagcagaag
aagtcagtagttattctcccatgaacgttcttgtctgtgtacagtttttagaacattacaaaggatctgtttgattagctgtcaacaa
aaagaaaacctgaaggagcatttggaagtcaatttgaggtttttttttttggtttttttttttttgtattttggaacgtgccccagaatg
aggcagttggcaaacttctcaggacaatgaatcttcccgtttttctttttatgccacacagtgcattgttttttctacctgcttgtctt
atttttagcataatttagaaaaacaaaacaaaggctgtttttcctaattttggcatgaacccccccttgttccaaaaatgaagacgg
catcatcacgaagcagctccaaaaggaaaagcttggcaggtgccctcgtcctggggacgtggagggtggcacggtcccc
gcctgcaccagtgccgtcctgctgatgtggtaggctagcaatattttggttaaaatcatgtttgtggccgaacgggcccctgc
acccg
DEFINITION Homo sapiens HMG-box containing protein 1 (HBP1), mRNA.
ACCESSION XM_027193 SEQ ID NO:51
Agcaccataacatggtgtgggaagtgaagacaaatcagatgcctatgcagtacagaaactcctgttggtgatggacaag
agagcctcaggaatgaatgactcattggagttgctgcagtgtaatgagaatttgccatcttcacctggatataactcctgtgat
gaacacatggagcttgatgaccttcctgaacttcaggcagttcaaagtgatcctacccaatctggcatgtaccagctgagttc
agatgtttcacatcaagaatacccaagatcatcttggaaccaaaatacctcagacataccagaaactacttaccgtgaaaatg
aggtggactggctaacagaattggcaaatatcgcgaccagtccacaaagtccactgatgcagtgctcattttacaatagatca
tctcctgtacacatcatagccactagcaaaagtttacattcctatgcacgccctccaccagtgtcctcttcttcgaagagtgaac
cagccttccctcatcaccattggaaggaggaaacaccagtaagacacgaaagggcaaatagtgagtcagaatctggcattt
tctgcatgtcctccctgtcagatgatgatgatttgggatggtgcaattcctggccttcaactgtctggcactgttttttgaaaggc
acacgactgtgctttcataagggaagcaataaggaatggcaagatgttgaagattttgctagagctgaaggctgtgataatga
ggaagatcttcaaatgggcattcacaagggctatggttctgatggtctaaagttgttatcacatgaagaaagtgtatcatttggc
gagtctgtactgaagttgacttttgatcctggtacagtagaagatggtttacttaccgtagagtgtaagctggaccaccctttct
atgttaaaaataaaggttggtcatcattttatccaagcttgactgtggtacagcatggcattccatgttgtgaagttcatattggc
gatgtatgtctacctactggacaccccgatgccattaattttgatgattcaggtgtttttgatacatttaaaagctatgacttcaca
cctatggattcttctgcagtttatgtgttaagtagtatggctcgccagcgtcgtgcatctttgtcttgtggaggacctggtggtca
agactttgcaagatctggattcagtaaaaactgtggctcacctggatcatcacagctctcttccaattctttgtatgctaaagctg
tcaaaaaccacagctcagggactgtgagtgccacttctcctaataagtgcaaaagaccaatgaatgccttcatgctttttgcca
aaaaatacagagttgaatatactcagatgtatccagggaaagataacagagccataagtgtgatccttggtgacaggtggaa
gaaaatgaagaatgaagagagaagaatgtacacattagaagcaaaggctttggctgaagaacagaaacgtttaaatcctga
ctgttggaagaggaaaagaaccaattcaggctcacaacaacattaaaccaggatgcttatgttcttaagtctatatttgcatata
cattgactcttgatggaaagacttagaagatcaaggtctcaccatcctcaattcgtgtgaccataagatactgatagcat
tgagtcttgaaatgatttaataatatgagtgaggatttgctttctccattagagcattaagctaaaactatcaacattttaaaccaa
attgccttatttttcttccaaacttcatatatgtctatcaggtaataataggcttgaaaattgatatcctgtggtgctaaagtacagt
agaaagagaggagaagtgtatacatgttttattttaaattgtacgaaaggggaatttaaaaaatatgtaactgctgtttataaatt
ggctccttactgcttattaatctgtattgtacacatgatgaaatgaagcagaagctgggagtcggcctttcctctagtaaccacc
acatggctcagcatctgtgccaaacataggcgctcctagtctggtcagtgccaagaggctaccagaacatggggcaggtg
gctggtgttggtgtcccagcctaagagccacctgctgcagttaccatggcatgctgagttgatgcaccaggtggcagcagc
catccgttattatttccaatggagacctagcccaggccaaggtaaagttagttaatagcattgggatatagtcactgtaatggtg
ctattaacaaacagtcaacaccattgtattttttaacttcgtgttctgtatctcctcagccatgtatcttaaatatattttgtcatcata
atctttatggtgggggcagactttgcacttactgcagtgcaacacttgcactttaattttcctccaactgtctaaaattagagcaa
atacattggcaatacagctgcttttgctctgagctacaatcatggcttttcatgttacttaccaagtggtgtttctggttaggaatc
acagctgtaaaattgatttcagttcatcacacttcttcatgatgttgcccctaaattttgcacactatattcttgtatattatttcaaat
aaatggaaaaa
DEFINITION Homo sapiens lymphoid enhancer factor-1 (LEF1) mRNA, complete
cds.
ACCESSION AF288571 SEQ ID NO:52
Aagatctaaaaacggacatctccaccgtgggtggctcctttttctttttctttttttcccacccttcaggaagtggacgtttcgtta
tcttctgatccttgcaccttcttttggggaaacggggcccttctgcccagatcccctctcttttctcggaaaacaaactactaagt
cggcatccggggtaactacagtggagagggtttccgcggagacgcgccgccggaccctcctctgcactttggggaggcg
tgctccctccagaaccggcgttctccgcgcgcaaatcccggcgacgcggggtcgcggggtggccgccggggcagcctc
gtctagcgcgcgccgcgcagacgcccccggagtcgccagctaccgcagccctcgccgcccagtgcccttcggcctcgg
ggcgggcgcctgcgtcggtctccgcgaagcgggaaagcgcggcggccgccgggattcgggcgccgcggcagctgctc
cggctgccggccggcggccccgcgctcgcccgccccgcttccgcccgctgtcctgctgcacgaacccttccaactctcctt
tcctcccccacccttgagttacccctctgtctttcctgctgttgcgcgggtgctcccacagcggagcggagattacagagccg
ccgggatgccccaactctccggaggaggtggcggcggcgggggggacccggaactctgcgccacggacgagatgatc
cccttcaaggacgagggcgatcctcagaaggaaaagatcttcgccgagatcagtcatcccgaagaggaaggcgatttagc
tgacatcaagtcttccttggtgaacgagtctgaaatcatcccggccagcaacggacacgaggtggccagacaagcacaaa
cctctcaggagccctaccacgacaaggccagagaacaccccgatgacggaaagcatccagatggaggcctctacaacaa
gggaccctcctactcgagttattccgggtacataatgatgccaaatatgaataacgaaccatacatgtcaaatggatctctttct
ccacccatcccgagaacatcaaataaagtgcccgtggtgcagccatcccatgcggtccatcctctcacccccctcatcactt
acagtgacgagcacttttctccaggatcacacccgtcacacatcccatcagatgtcaactccaaacaaggcatgtccagaca
tcctccagctcctgatatccctactttttatcccttgtctccgggtggtgttggacagatcaccccacctcttggctggcaaggt
cagcctgtatatcccatcacgggtggattcaggcaaccctacccatcctcactgtcagtcgacacttccatgtccaggttttcc
catcatatgattcccggtcctcctggtccccacacaactggcatccgtcatccagctattgtaacacctcaggtcaaacagga
acatccccacactgacagtgacctaatgcacgtgaagcctcagcatgaacagagaaaggagcaggagccaaaaagacct
cacattaagaagcctctgaatgcttttatgttatacatgaaagaaatgagagcgaatgtcgttgctgagtgtactctaaaagaa
agtgcagctatcaaccagattcttggcagaaggtggcatgccctctcccgtgaagagcaggctaaatattatgaattagcac
ggaaagaaagacagctacatatgcagctttatccaggctggtctgcaagagacaattatggtaagaaaaagaagaggaag
agagagaaactacaggaatctgcatcaggtacaggtccaagaatgacagctgcctacatctgaaacatggtggaaaacga
agctcattcccaacgtgcaaagccaaggcagcgaccccaggacctcttctggagatggaagcttgttgaaaacccagactg
tctccacggcctgcccagtcgacgccaaaggaacactgacatcaattttaccctgaggtcactgctagagagctgatccat
aaagacaatcactgccaacccctctttcgtctactgcaagagccaagttccaaaataaagcataaaaaggtttttaaaagga
aatgtaaaagcacatgagaatgctagcaggctgtggggcagctgagcagctctccccccaatctgcagcacaccca
gagcatcttgcatccaaacctgaacctttcggcaaggacggtaacttggctgcaagcctgtcatgcgcactggagccag
caaccagctatccatcagcaccccagtggaggagttcatgaagagttccctctttgtttctgcttcattttctttcttttcttttct
gggcaatgcagccggccctaaagcttttatttaacagtgcaaaaggatcgtgctttaaacttgaaataatttacagaactt
gtatattttgctagctatgagcttttaaataaaattgaaagttctggaaaagtttgaaataagacataaaagagccttcttttc
tgagacagcttgtctggtaagtggcttctctgtgaattgcctgtaacacatagtggcttctccgcccttgtaaggtgttcagtag
agctaaataaatgtaatagccaaaccccactctgttggtagcaattggcagccctatttcagtttattttttcttctgttttcttctttt
cttttttaaacagtaaaccttaacagatgcgttcagcagactggtttgcagtgaattttcatttctttccttatcaccccccttgttgt
aaaaagcccagcacttgaattgttattacttaaatgtctgtatttgtatctgttattagccaattagtgggataatgccaa
gttaaaatgagcattgatgtacccattaaaaaagcaagcacagcctttgcccctgtcatcctaacgttggtcaucca
gtttgagttaatgtgctgagcatttaaaagaagcttgtaataaaacattaaaaattgtcataaaaaaaa
a
DEFINITION Homo sapiens nemo-like kinase mRNA, complete cds.
ACCESSION AF197898 SEQ ID NO:53
Tttcccttttccgttggcaacccacacccttccacacacgctcacccgaaaattaaacaccaagatcctctaacttgag
gattgactgatgaagacataaagctctatgttttttgaggtggagtgagtggtttttcttcatttttaaatggccaaatgacagctt
gacccagtttgctttccaatcaaagggcatttattttgaatgtctctttgtggcgcaagagccaacgcaaaaatgatggcggctt
acaatggcggtacatctgcagcagcagcaggtcaccaccaccaccatcaccaccaccttccacacctccctcctcctcacc
tgcaccaccaccaccaccctcaacaccatcttcatccggggtcggctgccgctgtacaccctgtacagcagcacacctcnc
ggcagctgcggcagccgcagcagcggctgcagctgcagccatgttaaccctgggcaacacagccatawcccatca
cggcaccggggcaggctcctggaccagctgcagcagccccagctcaggtacaggctgccgcagctgctacagtaagg
cgcaccatcatcagcactcgcatcatccacagcagcagctggataagagccggatagactanggatatggagccagg
tgttgtctggtcagtaacagatccaagagatggaaagagagtagcgctcaaaaagatgcccaacgtcttccagaatctggtc
tcttgcaaaagggtcttccgggaattgaagatgttgtgttaaagcatgataatgtactctctgcccngacaactccaaacct
ccacacattgactattgaagaaatatatgtttcacagaattgatgcagagtgacctacataaanatcactctcctcaacc
actcagctcagatcatgtcaaagttctttatcagatwgcgaggttgaaatatctccattcagctggcaataatcgagac
aagccagggaatctccttgtgaacagcaactgtctagatttgtgataggattggccagaaggagatagatg
aatcccgtcatatgactcaggaagttgttactcagtattatcgggctccagaaatcctgatgggcagccgtcatacagcaatg
ctattgacatctggtctgtgggatgtatcttgcagaactactaggacgaagaatattgttcaggcacagaatcccattcagca
gttggatttgatcacggatctgttgggcacaccatcactggaagcaatgaggacagcttgtgaaggcgctaaggcacatata
ctcaggggtcctcataaacagccatctcttcctgtactctataccctgtctagccaggctacacatgaaagtcggtccac
caggatgttggtctttgatccatccaaaagaatatccgctaaggatgccuagcccacccctacctagatgagggcgactac
gatatcacacatgtatgaaatgttgcttccacctccactggaagagtttataccagtgactttgagcctgtcaccaatccc
aaatttgatgacactttcgagaagaacctcagttctgtccgacaggttaaagaaattattcatcagttcattttggaacagcaga
aaggaaacagagtgcctctctgcatcaaccctcagtctgctgctagagcwauaaccactatgctcagccatctga
gatgcccccatctcctctggtgtgggagtgatggtggaagataatgtactactgagatgtatgtagctttccactggaact
gggattgcaattctggaggttaatcatgcttgtactgtaattItactaatgaagttttaaattaacaaccactacttgtatgatatg
aataatatttagaaatgttactagacttttaatcttaaatgggcttttagaagaaaatattttacccagagttgcacatg
ttttatgaatttagtgcagctgttatggctcacctcagaacaaaagagaattgaaccaaatttgggagtttggggttttatgtttag
tttttcttttctaaaatgaagtgagattgttcacacacacacacacacacacacacacacacaaacacaaaggacagtcatac
attttgatatttgagccattcctaaagatttggggtctaaaactaaagaaatctaggaccttgcctgcgaccaatcatggagc
cacgtgagctgatcgtggctgcacctggggggagggtagggaggaggggcatgccacctaatgatcaaagccctataatta
gcttctcattagagccgtgatggtgatgtgtgctgtctaatccaatgttgtggaagagagaatgagagtgactaggaga
gactaaacttttgttttccttacccagtataaatatatatatatatatttaatctatttttattagaagtttttctgctctttcttacataaa
agaaccccaagcatgcatctttcatgtgtgtaaataattcatttctgggctaatttcaaaagaatcccaatattgctgtatagaaa
gagaactagcttgcacatttaggtctgtgaattttgtgagacttttcctgcactggacagtaaaaaataataaaagacaaaa
acaaatttaaaaaaaaaa
DEFINITION Homo sapiens Pontin52 mRNA, complete cds.
ACCESSION AF099084 SEQ ID NO:54
Cgggcgggcgcactgtcctagctgctggttttccacgctggttttagctcccggcgtctgcaaaatgaagaugaggaggtg
aagagcactacgaagacgcagcgcatcgcctcccacagccacgtgaaagggctggggctggacgagagcggcttggcc
aagcaggcggcctcagggcttgtgggccaggagaacgcgcgagaggcataggcgtcatagtagaaaatcaaagca
agaaaatggctggaagagctgtcttgttggcaggacctcctggaactggcaagacagctctggctctggctattgctcagga
gctgggtagtaaggtccccttctgcccaatggtggggagtgaagtttactcaactgagatcaagaagacagaggtgctgatg
gagaacttccgcagggccattgggctgcgaataaaggagaccaaggagntatgaaggtgaaacacagagctaactac
gtgtgagacagagaatcccatgggaggatatggcaaaaccattagccatgtgatcataggactcaaaacagccaaaggaa
ccaaacagttgaaactggaccccagcatttttgaaagtttgcagaaagagcgagtagaagctggagatagatttacatgaa
gccaacagtggggccgtgaagaggcagggcaggtgtgatacctatgccacagaaucgaccttgaagctgagagtatgt
ccccttgccaaaaggggatgtgcacaaaaagaaagaaatcatccaagatgtgaccttgcatgacuggataggctaatgc
gcggccccaggggggacaagatatcctgtccatgatgggccagctaatgaagccaaagaagacagaaatcacagaca
acttcgaggggagattaataaggtggtgaacaagtacatcgaccagggcaugctgagctggtcccgggagctagtt
gatgaggtccacatgctggacattgagtgcttcacctacctgcaccgcgccctggagtcttctatcgctcccatcgtcatctta
gcatccaaccgaggcaactgtgtcatcagaggcactgaggacatcacatcccctcacggcatccctcttgaccuctggacc
gagtgatgataatccggaccatgctgtatactccacaggaaatgaaacagatcattaaaatccgtgcccagacggaaggaa
tcaacatcagtgaggaggcactgaaccacctgggggagattggcaccaagaccacactgaggtactcagtgcagctgctg
accccggccaacttgcttgctaaaatcaacgggaaggacagcattgagaaagagcatgtcgaagagatcagtgaacuttct
atgatgccaagtcctccgccaaaatcctggctgaccagcaggataagtacatgaagtgagatggctgaggttttcagcagc
aagagactccccaggtgtgcctggcctgggtccagcctgtgggcgcttgcccctgggcttggggctgccaccccactca
ggcgtgggctgcagcgctacaatcagtgtggaaagcatttctttaaagttatcgtaactgttcctgtggttgcgtgaaaga
cccttccttacctggtgtgttttctataaatcttcataggttattttgatcttctctctctctctctaagttttttaaaaataaactttcag
aacagtaaaaaaaaaaaaaa
DEFINITION Homo sapiens Reptin52 mRNA, complete cds.
ACCESSION AF124607 SEQ ID NO:55
Gcggccgcgtcgacgagcatcatggcaaccgttacagccacaaccaaagtcccggagatccgtgatgtaacaaggattg
agcgaatcggtgcccactcccacatccggggactggggctggacgatgccttggagcctcggcaggcucgcaaggcat
ggtgggtcagctggcggcacggcgggcggctggcgtggtgctggagatgatccgggaagggaagattgccgacgggc
agtccttattgctggccagccgggcacggggaagaaggccatcgccatgggcatggcgcaggccctgggccctgacac
gccattcacagccatcgccggcagtgaaatcttctccctggagatgagcaagaccgaggcgctgacgcaggccttccggc
ggtccatcggcgttcgcatcaaggaggagacggagatcatcgaaggggaggtgaggagatccagatgatcgaccagc
aacagggacgggctccaaggtgggcaaactgaccctcaagaccacagagatggagaccatctacgacctgggcaccaa
gatgattgagtccctgaccaaggacaaggtccaggccggggacgtgatcaccatcgacaaggcgacgggcaagatctcc
aagctgggccgctccttcacacgcgcccgcgactacgacgctatgggctcccagaccaaatcgtgcaagcccagatgg
ggagctccagaaacgcaaggaggtggtgcacaccgtgtccctgcacgagatcgacacatcaactctcgcacccagggc
ttcctggcgctcttctcagagacacaggggagatcaaacagaaaccgtgagcagatcaatgccaagaggctgagtgg
cgcgaggagggcaaggcggagatcatccctggagtgctgttcatcgacgagaccacatgctggacatcgagagcuctc
cttcctcaaccgggccctggagagtgacatggcgcctgtcctgatcatggccaccaaccaggcatcacgcgaatccggg
gcaccagctaccagagccctcacggcatccccatagacctgctggaccggctgcuatcactccaccaccccctacagcg
agaaagacacgaagcagatcctccgcatccggtgcgaggaagaagataggagatgaagaggacgcctacacggtgct
gacccgcatcgggctggagacgtcactgcgctacgccatccagctcatcacagctgccagcttggtgtgccggaaacgca
agggtacagaagtgcaggtggatgacatcaagcgggtctactcactcttcctggacgagtcccgctccacgcagtacatga
aggagtaccaggacgccttcctcUcaacgaactcaaaggcgagaccatggacacctcctgagttggatgtcatccatcccccga
ccccaccctgttttccaccagagttctgacactgtgactctgtataaaatggttgggaagctgcaaaaaaaaaaaaaaaaa
aaa
Wnt target genes:
DEFINITION Homo sapiens achaete-scute complex (Drosophila) homolog-like 1
(ASCLL), mRNA.
ACCESSION NM_004316 SEQ ID NO:56
Cccgagacccggcgcaagagagcgcagccttagtaggagaggaacgcgagacgcggcagagcgcgttcagcactga
cttttgctgctgcttctgcttttttttttcttagaaacaagaaggcgccagcggcagcctcacacgcgagcgccacgcgaggat
cccgaagccaacccgcgaagggaggaggggagggaggaggaggcggcagcagggaggagaaaaagaatacaatt
ttttttgctcccactctaagaagtctcccggggattttgtatatattttttaacttccgtcagggctcccgcttcatautccttttca
ccctctctgttcctgcacccaagttctctctgtgtccccctcgcgggccccgcacctcgcacccggatcgctctgattccgc
gactccttggccgccgctgcgcatggaaagctctgccaagatggagagcggcggcgccggccagcagccccagccgca
gccccagcagcccttcctgccgcccgcagcctgtttctttgccacggccgcagccgcggcggccgcagccgccgcagcg
gcagcgcagagcgcgcagcagcagcagcagcagcagcagcagcagcagcagcagcaggcgccgcagctgagaccg
gcggccgacggccagccctcagggggcggtcacaagtcagcgcccaagcaaacaagcgacagcgctcgtcucgccc
gaactgatgcgctgcaaacgccggctcaacttcagcggctttggctacagcctgccgcagcagcagccggccgccagg
cgcgccgcaacgagcgcgagcgcaaccgcgtcaagttggtcaacctgggcutgccacccucgggagcacaaaacaa
cggcgcggccaacaagaagatgagtaaggtggagacactgcgctcggcggtcgagtacatccgcgcgctgcagcagct
gctggacgagcatgacgcggtgagcgccgccttccaggcaggcgtcctgtcgcccaccatctcccccaactactccaaacg
acttgaactccatggccggctcgccggtctcatcctactcgtcggacgagggctcttacgacccgctcagccccgaggagc
aggagcttctcgacttcaccaactggttctgaggggctcggcctggtcaggccctggtgcgaatggacmggaagcaggg
tgatcgcacaacctgcatctttagtgctttcttgtcagtggcgttgggagggggagaaaaggaaaagaaaaaaaaagaaga
agaagaagaaaagagaagaagaaaaaaacgaaaacaacaaccaaccccatcgccaactaagcgaggcatgcctgaga
gacatggctttcagaaaacgggaagcgctcagaacagtatcttgcactccatcaacacggagatatgagagcaactgg
gacctgagtcaatgcgcaaaatgcagcttgtgtgcaaaagcagtgggctcctggcagagggagcagcacaagcattata
gtaactcccatcacctctaacacgcacagctgaaagttcttgctcgggtcccttcacctccccgccctucuagaagcagtt
cttagccctctagaaacgagttggtgtctttc
DEFINITION Homo sapiens achaete-scute complex-like 2 (Drosophila) (ASCL2),
mRNA.
ACCESSION NM_005170 SEQ ID NO:57
Gggcgtgagaaaggcgacggcggcggcgcggaggagggttatctatacautaaaaccagccgcctgcgccgcgcct
gcggagacctgggagagtccggccgcacgcgcgggacacgagcgtcccacgctccctggcgcaacggcctgccacc
actaggcctcctatccccgggctccagacgacctaggacgcgtgccctggggaatgcctggcggcgccgtgccagaag
cccccttggggcgccacagttttccccgtcgcctccggttcctctgcctgcaccttcctgcggcgcgccgggacatggagc
gggcgggtggatgcaggcgcgatggacggcggcacactgcccaggtccgcgccccctgcgccccccgtccctacggc
tgcgctgcccggcggagacccgcgtccccggaactgttgcgctgcagccggcggcggcgaccggccaccgcagagac
cggaggcggcgcagcggccgtagcgcggcgcaatgagcgcgagcgcaaccgcgtgaagctggtgaacugggcttcc
aggcgctgcggcagcacgtgccgcacggcggcgccagcaagaagctgagcaaggtggagacgctgcgctcagccag
gagtacatccgcgcgctgcagcgcctgctggccgagcacgacgccgtgcgcaacgcgctggcgggagggctgaggcc
gcaggccgtgcggccgtctgcgccccgcgggccgccagggaccaccccggtcgccgcctcgccctcccgcgcucuc
gtccccgggccgcgggggcagctcggagcccggctccccgcgttccgcctactcacggacgacagcggctgcgagg
cgcgctgagtcctgcggagcgcgagctactcgacttctccagctggttagggggctactgagcgccctcgaccta
DEFINITION Homo sapiens fibroblast growth factor 5 (FGF5), mRNA.
ACCESSION XM_054589 SEQ ID NO:58
Tctcttcccctctccccttctcttccccgaggctatgtccacccggtgcggcgaggcgggcagagccagaggcacgcagc
cgcacaggggctacagagcccagaatcagccctacaagatgcacttaggacccccgcggctggaagaatgagcttgtcct
tcctcctcctcctcttcttcagccacctgatcctcagcgcctgggctcacggggagaagcgtctcgcccccaaagggcaac
ccggacccgctgccactgataggaaccctagaggctccagcagcagacagagcagcagtagcgctatgtcttcctcttctg
cctcctcctcccccgcagcttctctgggcagccaaggaagtggcttggagcagagcagtttccagtggagcccctcgggg
cgccagaccggcagcctctactgcagagtgggcatcggtttccatctgcagatctacccggatggcaaagtcaatggatcc
cacgaagccaatatgttaagccaagttcacagatgactgcaagttcagggagcgttttcaagaaaatagctataatacctatg
cctcagcaatacatagaactgaaaaaacagggcgggagtggtatgtggccctgaataaaagaggaaaagccaaacgagg
gtgcagcccccgggttaaaccccagcatatctctacccattttctgccaagattcaagcagtcggagcagccagaactttcttt
cacggttactgttcctgaaaagaaaaagccacctagccctatcaagccaaagattcccctttctgcacctcggaaaaatacca
actcagtgaaatacagactcaagtttcgctttggataatattcctcttggccttgtgagaaaccattctttcccctcaggagtttct
ataggtgtcttcagagttctgaagaaaaattactggacacagcttcagctatacttacactgtattgaagtcacgtcatttgtttc
aatgtgactgaaacaaaatgttttttgataggaaggaaactggaattctttgtactaatacagggagcacactccttcagttcag
caagacataaagccttttgctttatgattgagggatatttagaactttgtattttcggaaagttaaataacagggactacgtattttt
ctgacttttacagattaacctgaaagaacatacatgatacatttttatttttggtttccaaagaatattttgatgcagataaaatatttt
gttaacttttgtttttttttgtttgttQcttaaaagtacctctgcattgagcatattttcttacttttattattttaattaatatgacataagc
aatcattttatgctgtttatgaattataaatgtgtttatagctcatttgtaatatggaaatcttttacatttttcctattcactgcactttttt
attgtttttatttctagccatacctcagataatatgtttagttttacattttaaaatgtttaaattctctttcacagcaccaaaggctca
gcttggatttgtgtgtatgtgtatgtcaattcatgacattatgtggaatcctaaacctttggtggctgggatatgatgggttagaa
gcaaggagaaaatataaggactttttgatggaattaaatgtgggaggtaaggaaaaggatttagaggtaaaagtacactaag
tttgcaacatttattgagatctaagtctgtcttgccttcatttctctttttatctcccccttgccctcattcttgaacagctggaggaat
acattttattctgtccatgaagcatacactatgaaattcaagtgcttaaaaatacttctatgactctctgctatcccactgtatagat
ccacagggagcaaacacttagaaatgatagagaactgaaggagatcaatggtttaacagttatccatgccaagtcccattgt
cagaaatattcttattactcagtcaaacactctttgagcttcccttcctaaaggtaaccaatccagtgaatagatgtgcccttttat
aaggaaacttctgatgtttattaaaaaaactggccttttgatagaggtaacttaatttgggaatttgttgtgttgaaatggcatttaa
tttcaacctaaatactgactgctggacataaatcacagaaaatttaacttaagaaaatttacaaaatttattctcaggtaatcatttt
aataaagttctgcaaaatacacgtttatcttacattcagaaatgtggcaaaaaaggcatagctaaaggctaaacatatggcttt
agtagtaacaaaagggttcatagaaacttcatggtttgcatttaaacatgtttaaagtgtacttataaactattt-ttttcttaaagca
aactatgatttattttggtgcacaaatacaaagtggaaacttaccaaaattgaactagctaccatataagcagattgctttaattt
gatgggaaaatagtacacacatatatataacaaataatatattaaaaaacccatccatcaactaaaacattatatgtatacatca
gtatagtgttttattataaagccaattatctgattaagcattctttccactgaatgcataatgtttaaatagcataaaatgaaatgct
acaaaaattgaactaatttatactttaaagtatttctgggttaaatgaaacaatgaaattttttagtatgttcaactctcatccaaatg
gcatatgaccctgtttacacagcctaaagctaaaaatattactctagtttattctaatctattgttaagtattgtgcactgtatacca
agttcttagggcacatgaaaaattttagctgccaaacaggaactagtaaacatatgttcctaataagtgaagggaaagataat
aatgatggtcaacaataagccacgtaaatgcataagttgtataggctaaatgttgcttgtaggctacattaaactcaaatgtaat
agtttatcttatactcctggtttgatttgattagcatattaacgtgaaagtaggatagctactaaatatatattatgcaagtcaggaa
tcattaatttcaaaatttaaagccatgctaaaattaaaaagaaaatattaaattacacaattacacttgtctttactggccatacaa
aatgattttttttttttttttgagacagagtcttgctctgtcaccaggctggagtgcagtggcatgatctcggctcactgcaacctc
caactccctggtttaagggattctcctgcctcagcctcccaagtagctgggattacagactcatgccaccacgccagctaattt
ttgtatttttagtagagacggggtttcaccatgttggtcaggatggtctcaatcctggcctcttgatagtcctgacctcatgatct
gcccacctcggcctccccaaagtgctgggattacaggtacaatgatgtataattaatgcttagtgaagcataaagttacctac
atcaattaattaaatgaacttatgtacagaaaacatgtataaatataagtctatactaatgcttacaactttctaagagggttcttg
cttatgtagctttttattattttaagtaactagaaccaccaaatatcaaataaaattatttggttatggttatgttcatctaaacacaa
caataacttttatattaatatttaggagtctattttgtctataggtgacaaacatctccagactaacatgtcagttttatcaattatatt
atgtttaattatttaagatttctttatgtggaacatctatagagataaatagaaattttcaataagatgtagtaacactgtgatttatct
ttcaagagtctctcttcacttccttctaaagagactaatttgagagtacaggtgcatattaattttcttggttctttcagctgaattat
attggtccagaagttcaaaatcatgtgacaataataagggatactgacagaagttatttccaagtttgtgtatatattataaaaatt
acatatataaaactaaggcttttatttctgttatttttaagcttttatttcttgtagctaaaaataaaacatcataaatctggtaggtaa
atttcttattaaatcaatcttgaaatagaaaatgtaataactttcttaccattaacattttttacccttccatagaagggagggaata
aatcatgacttatcccattttcaataacaaaacgaaactatggcactaaccaaaaacttgaattctggcataatttttacagttgc
agagaattgtttctgggctcattaaaaaaagtagtattgcagacattgctgcaatgggaagcagacaataacttcttaaaggaa
ttctacacctcctttaagatttacttaattgctacatctaaattctgataatttaaaatccattttaggtgataaaattttttaaaagtttt
gaaggaaacctctggataaatggacaaggcctaatttttttttgtagtcaatccaactgtactggccaatttttgaaataagatta
tatgattaggtattagcagagacaaagagttacctcctccatcttactctgccctatttgaaagtctcaggggagaaaagggaa
caagatgctgatccaacctgagtggagtcaggtgaggcatctttacatctaagaatttttttttaaattttattattattatacttcaa
gttctagggtacatgtccacaatgcacatgtctgtcacacatgcacacatgtgccatgctggtgtgctgcacccaccaacctg
tcatccagcattaggtatatctcctaatgctatccctcccctctccacccaccccacagcaggccccggtatgtgatgttcccc
ttcgtgtgtccatgtgttcttattgttcaattcccacctatgagtgagaatatgtggtgtttggtttttggtccttgcaatagtttgctg
agaatgatggtttccagcttcatccatgtccctacaaagaacatgaactcatcatttttgatggctgcatagtattccatggtgta
tatgtgccacattttcttatccagtctatcattgttggacatttgggttggttccagtctttgctattgtgaatagtgctgcaataaa
catatgtgtg
DEFINITION Homo sapiens muscle segment homeobox 1 (MSX1) gene, complete
cds.
ACCESSION AF426432 SEQ ID NO:59
Tgcgtgcccccaggcccagcgcgcctccgggcgagtccccaggagcgcggcccaatggatcgctccgggcccgcccc
ctcgcgcgctgattggccgccgccccgctggcctcgccttattagcaagttctctggggagccgcggtagggcccggagc
cggcgagtgctcccgggaactctgcctgcgcggcggcagcgaccggaggccaggcccagcacgccggagctggcctg
ctggggaggggcgggaggcgcgcgcgggagggtccgcccggccagggccccgggcgctcgcagaggccggccgc
gctcccagcccgcccggagcccatgcccggcggctggccagtgctgcggcagaagggggggcccggctctgcatggc
cccggctgctgacatgacttctttgccactcggtgtcaaagtggaggactccgccttcggcaagccggcggggggaggcg
cgggccaggcccccagcgccgccgcggccacggcagccgccatgggcgcggacgaggagggggccaagcccaaag
tgtccccttcgctcctgcccttcagcgtggaggcgctcatggccgaccacaggaagccgggggccaaggagagcgccct
ggcgccctccgagggcgtgcaggcggcgggtggctcggcgcagccactgggcgtcccgccggggtcgctgggagccc
cggacgcgccctcttcgccgcggccgctcggccatttctcggtggggggactcctcaagctgccagaagatgcgctcgtc
aaagccgagagccccgagaagcccgagaggaccccgtggatgcagagcccccgcttctccccgccgccggccaggcg
gctgagccccccagcctgcaccctccgcaaacacaagacgaaccgtaagccgcggacgcccttcaccaccgcgcagct
gctggcgctggagcgcaagttccgccagaagcagtacctgtccatcgccgagcgcgcggagttctccagctcgctcagcc
tcactgagacgcaggtgaagatatggttccagaaccgccgcgccaaggcaaagagactacaagaggcagagctggaga
agctgaagatggccgccaagcccatgctgccaccggctgccttcggcctctccttccctctcggcggccccgcagctgtag
cggccgcggcgggtgcctcgctctacggtgcctctggccccttccagcgcgccgcgctgcctgtggcgcccgtgggact
ctacacggcccatgtgggctacagcatgtaccacctgacatagagggtcccaggtcgcccacctgtgggccagccgattc
ctccagccctggtgctgtacccccggacgtgctcccctgctcggcaccgccagccgccttccctttaaccctcacactgctc
cagtttcacctctttgctccctgagttcactctccgaagtctgatccctgccaaaaagtggctggaagagtcccttagtactctt
ctagcatttagagatctaccctctcgagttaaaagatggggaaactgagggcagagaggttaacagatttatctaaggtcccc
agcagaattgacagtgagaacagagctagaggccatgtctcctgcatagtttttccctgtcctgacaccaggcaagaaagcg
cagagaaatcggtgtctgacgattttggaatgagaacaatctcaaaaaaaaaaaaaaaaaaaaaaaaaagaaaagagaaa
aaaaagactagccaccaggaagatgaatcctagcttcttccattggaaaatttaaagacaagttcaacaacaaaacatttgct
ctggggggcagggaaaacacagatgtgttgcaaaggtaggttgaaggga
DEFINITION Homo sapiens neurogenin 1 (NEUROG1), mRNA.
ACCESSION NM_006161 SEQ ID NO:60
Atgccagcccgccttgagacctgcatctccgacctcgactgcgccagcagcagcggcagtgacctatccggcttcctcac
cgacgaggaagactgtgccagactccaacaggcagcctccgcttcggggccgcccgcgccggcccgcaggagcgcgc
ccaatatctcccgggcgtctgaggttccaggggcacaggacgacgagcaggagaggcggcggcgccgcggccggacg
cgggtccgctccgaggcgctgctgcactcgctgcgcaggagccggcgcgtcaaggccaacgatcgcgagcggaaagg
catgcacaacttgaacgcggccctggacgcactgcgcagcgtgctgccctcaccccgacgacaccaagctcaccaaaa
tcgagacgctgcgcttcgcctacaactacatctgggctctggccgagacactgcgcctggcggatcaagggctgcccgga
ggcggtgcccgggagcgcctcctgccgccgcagtgcgtcccctgcctgcccggtcccccaagccccgccagcgacgcg
gagtcctggggctcaggtgccgccgccgcctccccgctctctgaccccagtagcccagccgcctccgaagacttaaacta
ccgccccggcgaccctgttttctccttcccaagcctgcccaaagacttgctccacacaacgccctattcauccaaacaaaa
g
DEFINITION Homo sapiens neurogenin 2 gene, partial cds.
ACCESSION AF303002 SEQ ID NO:61
Ggcctcccccgccttggcggccctgaccccgctgtcatccagcgccgacgaagaagaggaggaggagccgggcgca
caggcggggcgcgtcggcagcgcggggctgaggccgggcagggggcgcggggcggcgtggctgcggagcggag
ggctgccggcccgcacggctgctgggtctggtacacgattgcaaacggcgcccttcccgggcgcgggacactcccgag
gcgccaagacggccgagacggtgcagcgcatcaagaagacccgtagactgaaggccaacaaccgcgagcgaaaccg
catgcacaacctcaacgcggcactggacgcgctgcgcgaggtgctccccacaccccgaggacgcGaagctcaccaag
atcgagaccctgcgcttcgcccacaactacatctgggcactcaccgagaccctgcgcctggcggatcactgcgggggcg
gcggcgggggcctgccgggggcgctcttctccgaggcagtgttgctgagcccgggaggagccagcgccgccctgagc
agcagcggagacagcccctcgcccgcctccacgtggagttgcaccaacagccccgcgccacctcctccgtgtactaaaa
ttccacctccccctacagctgcactttatcgcccgccagcccggccgggtcagacatggactattggcagcccccacctcc
cgacaagcaccgctatgcacctcacctccccatagccagggaagtatctag
DEFINITION Homo sapiens neurogenin 3 (NEUROG3), mRNA.
ACCESSION XM_005744 SEQ ID NO:62
Cctcggaccccattctctcttcttttctcctttggggetggggcaactcccaggcgggggcgcctgcagctcagctgaacttg
gcgaccagaagcccgctgagctccccacggccctcgctgctcatcgctctctauctutgcgccgaagaaaggatgaag
cctcaaccctcgggtgcgcccactgtccaagtgacccgtgagacggagcggtccuccccagagcctcggaagaagaag
tgacctgccccacgtccgccccgcccagccccactcgcacacgggggaactgcgcagaggcggaagagaaggatga
cgaggggccccgaggaagctccgggcacggcgcgggggacgcagccggcctaagagcgagaggcactgagcaaga
agcgacggagtcggcgaaagaaggccaacgaccgcgagcgcaatcgaatgcacaacctcaactcggcactggacgcc
ctgcgcggtgtcctgcccaccttcccagacgacgcgaagctcaccaagatcgagacgctgcgcacgccaacaaataaaa
ctgggcgctgactcaaacgctgcgcatagcggaccacagcttgtacgcgctggagccgccggcgccgcactgcgggga
ctgggcgctgactcaaacgctgcgatagcggaccacagcttgtacgcgctggagccgccggcgccgcactgcgggga
gctgggcagcccaggcggttcccccggggactgggggtccctctactccccagtctcccaggctggcagcctgagtccc
gccgcgtcgctggaggagcgacccgggctgctgggggccaccttttccgcctgcttgagcccaggcagtctggctttctca
gattttctgtgaaaggacctgtctgtcgctgggctgtgggtgctaagggtaagggagagggagggagccgggagccgtag
agggtggccgacggcggcggccctcaaaagcacttgttccttctgcttctccctggctgacccctggccggcccaggcctc
cacgggggcggcaggctgggttcattccccggccctccgagccgcgccaacgcacgcaacccttgctgctgcccgcgc
gaagtgggcattgcaaagtgcgctcattttaggcctcctctctgccaccaccccataatctcattcaaagaatactagaatggt
agcactacccggccggagccgcccaccgtcttgggtcgccctaccctcactca
DEFINITION Homo sapiens phosphatase and tensin homolog (mutated in multiple
Advanced cancers 1) (PTEN), mRNA.
ACCESSION NM_000314 SEQ ID NO:63
Cctcccctcgcccggcgcggtcccgtccgcctctcgctcgcctcccgcctcccctcggtcttccgaggcgcccgggctcc
cggcgcggcggcggagggggcgggcaggccggcgggcggtgataggcaggaatcaatgagctgcggcaggatac
gcgctcggcgctgggacgcgactgcgctcagttctctcctctcggaagctgcagccatgatggaagtttgagagttgagcc
gctgtgaggcgaggccgggctcaggcgagggagatgagagacggcggcggccgcggcccggagcccctctcagcgc
ctgtgagcagccgcgggggcagcgccctcggggagccggccggcctgcggcggcggcagcggcggcatctcgcct
cctcttcgtcttttctaaccgtgcagcctcttcctcggcttctcctgaaagggaaggtggaagccgtgggctcgggcgggag
ccggctgaggcgcggcggcggcggcggcggcacctcccgctcctggagcgggggggagaagcggcggcggcggcg
gccgcggcggctgcagctccagggagggggtctgagtcgcctgtcaccatttccagggctgggaacgccggagagugg
tctctccccttctactgcctccaacacggcggcggcggcggcggcacatccagggacccgggccgautaacctcccgt
ccgccgccgccgcaccccccgtggcccgggctccggaggccgccggcggaggcagccacggaggattattcacuc
tccccattccgctgccgccgctgccaggcctctggctgctgaggagaagcaggcccagtcgctgcaaccatccagcagcc
gccgcagcagccattacccggctgcggtccagagccaagcggcggcagagcgaggggcatcagctaccgccaaacc
agagccatttccatcctgcagaagaagccccgccaccagcagcttctgccatctctctcctcctuucucagccacaggctc
ccagacatgacagccatcatcaaagagatcgttagcagaaacaaaaggagatatcaagaggatggaucgacuagacag
acctatatttatccaaacattattgctatgggatttcctgcagaaagacttgaaggcgtatacaggaacaatattgatgatgtagt
aaggtttttggattcaaagcataaaaaccattacaagatatacaatctttgtgctgaaagacauatgacaccgccaaatttaatt
gcagagttgcacaatatccttttgaagaccataacccaccacagctagaacttataaaacccttttgtgaagatcttgaccaat
ggctaagtgaagatgacaatcatgttgcagcaattcactgtaaagctggaaagggacgaactggtgtaatgatatgtgcatat
ttattacatcggggcaaatttttaaaggcacaagaggccctagatttctatggggaagtaaggaccagagacaaaaaggga
gtaactattcccagtcagaggcgctatgtgtattattatagctacctgttaaagaatcatctggattatagaccagtggcactgtt
gtttcacaagatgatgtttgaaactattccaatgttcagtggcggaacttgcaatcctcagtttgtggtctgccagctaaaggtg
aagatatattcctccaattcaggacccacaGgacgggaagacaagttcatgtactttgaaccctcagccattacctagta
gagatatcaaagtagagttcttccacaaacagaacaagatgataaaaaggacaaaatatcactttgggtaaatacattc
ttcataccaggaccagaggaaacctcagaaaaagtagaaaatggaagtctatgtgatcaagaaatcgatagcatttgcagta
tagagcgtgcagataatgacaaggaatatctagtacuactttaacaaaaaatgatcttgacaaagcaaataaagacaaagcc
aaccgatacttttctccaaattttaaggtgaagctgtacttcacaaaaacagtagaggagccgtcaaatccagaggctagcag
ttcaacttctgtaacaccagatgttagtgacaatgaacctgatcattatagatattctgacaccactgactctgatccagagaat
gaaccttttgatgaagatcagcatacacaaattacaaaagtctgaatttttttttatcaagagggataaaacaccatgaaaataa
acttgaataaactgaaaatggacctttttttttttaatggcaataggacattgtgtcagattaccagttataggaacaattctctttt
cctgaccaatcttgtttaccctatacatccacagggttttgacacttgttgtccagttgaaaaaaggttgtgtagctgtgtcatgt
atatacctttttgtgtcaaaaggacatttaaaattcaattaggattaataaagatggcactttcccgttttattccagttttataaaaa
gtggagacagactgatgtgtatacgtaggaattttttccttttgtgttctgtcaccaactgaaaggctaaagagcutgtgatat
actggttcacatcctacccctttgcacttgtggcaacagataagtttgcagaggctaagagagatccgaaagamgctac
cattctaatgcatgtattcgggttagggcaatggaggggaatgctcagaaggaaataauatgctggactctggaccatat
accatctccagctautacacacacctttctttagcatgctacagttauaatctggacattcgaggaauggccgctgicactgc
ttgttgtttgcgcatttttttttaaagcatatggtgctagaaaaggcagctaaaggaagagaatctaattgggaacaggaatg
aaccttctgcaacatcttaagatccacaaatgaagggatataaaaataatgtcataggtaagaaacacagcaacaatgactta
accatataaatgtggaggctatcaacaaagaatgggcttgaaacattataaaaattgacaatgatttattaaatatgatctcaa
ttgtaaaaaaaaaa

TABLE 2

Polycomb Group.

EED
SEQ ID NO:64
>gi | 14523051 | ref | NM_003797.1 | Homo sapiens embryonic ectoderm development
(EED), Mrna
GAGGGAAGTGTCGACTGCGCCGGCGGGAACAGACATGCCTGCGGCCAAGAAGCAGAACTGAGACAGTGAC
GAGAACAGCAATCCAGAACTCTCTGGAGACGAGAATGATGACGCTGTCAGTATAGAAAGTGGTACAAACA
CTGAACGCCCTGATACACCTACAAACACGCCAAATGCACCTGGAAGGAAAAGTTGGGGAAAGGGAAAATG
GAAGTCAAAGAAATGCAAATATTCTTTCAAATGTGTAAATAGTCTCAAGGAAGATCATAACCAACCATTG
TTTGGAGTTCAGTTTAACTGGCACAGTAAAGAAGGAGATCCATTAGTGTTTGCAACTGTAGGAAGCAACA
GAGTTACCTTGTATGAATGTCATTCACAAGGAGAAATCCGGTTGTTGCAATCTTACGTGGATGCTGATGC
TGATGAAAACTTTTACACTTGTGCATGGACCTATGATAGCAATACGAGCCATCCTCTGCTGGCTGTAGCT
GGATCTAGAGGCATAATTAGGATAATAAATCCTATAACAATGCAGTGTATAAAGCACTATGTTGGCCATG
GAAATGCTATCAATGAGCTGAAATTCCATCCAAGAGATCCAAATCTTCTCCTGTCAGTAAGTAAAGATCA
TGCTTTACGATTATGGAATATCCAGACGGACACTCTGGTGGCAATATTTGGAGGCGTAGAAGGGCACAGA
GATGAAGTTCTAAGTGCTGATTATGATCTTTTGGGTGAAAAAATAATGTCCTGTGGTATGGATCATTCTCTTAAACT
TTGGAGGATCAATTCAAAGAGAATGATGAATGCAATTAAGGAATCTTATGATTATAATCCAAATAAAACTAACAGGC
CATTTATTTCTCAGAAAATCCATTTTCCTGATTTTTCTACCAGAGACATACATAGGAATTATGTTGATTGTGTGCGA
TGGTTAGGCGATTTGATACTTTCTAAGTCTTGTGAAAATGCCATTGTGTGCTGGAAACCTGGCAAGATGGAAGATGA
TATAGATAAAATTAAACCCAGTGAATCTAATGTGACTATTCTTGGGCQATTTGATTACAGCCAGTGTGACATTTGGT
ACATGAGGTTTTCTATGGATTTCTGGCAAAAGATGCTTGCATTGGGCAATCAAGTTGGCAAACTTTATGTTTGGGAT
TTAGAAGTAGAAGATCCTCATAAAGCCAAATGTACAACACTGACTCATCATAAATGTGGTGCTGCTATTCGACAAAC
CAGTTTTAGCAGGGATAGCAGCATTCTTATAGCTGTTTGTGATGATGCCAGTATTTGGCGCTGGGATCGACTTCGAT
AAAATACTTTTGCCTAATCAAAATTAAAATGTGTTTGTTGTCTGTGTAAAATAAAATTAATGTATCTTGCTAGTAAG
GGCACGTAGAGCATTTAGAGTTGTCTTTCAGCATTCAATCAGGTGAGCTGAATGTAGTGATGTTTACATTGTTTAGC
ATTCTTTGTACTGTCTTCCTGCTCAGACTCTACTGCTTTTAATAAAAATTTATTTTG
YY1
SEQ ID NO:65
>gi | 6042207 | ref | NM_003403.2 | Homo sapiens YY1 transcription factor (YY1),
mRNA
AGGGCGAACGGGCGAGTGGCAGCGAGGCGGGGCGGGdTGAGGCCAGCGCGGAAGTCTCGCGAGGCCGGGC
CCGAGCAGAGTGTGGCGGCGGCGGCGAGATOTGGGCTCGGGTTGAGGAGTTGGTATTTGTGTGGAAGGAG
GCGGAGGCGCAGGAGGAGGAAGGGGGAAGCGGAGCGCGGCCCGGACGGGAGGAGGCGCGGCCAGGGCGGG
CGGTTGCGGCGAGGCGAGGCGAGGAGGGGGAGCCGAGACGCAGCGGCCGAGGCGGGGGCGCGGGCGCACC
GAGGCGAGGGAGGCGGGGAAGCCCCGCCGCCGCCGCGGCGCCCGCCCCTTCCCCCGCCGCCCGCGCCCTC
TCCCCCCGCCGCGCTCGCCGCCTTCCTCCCTCGCTTCCTTCCCCACGGCCGGCCGCCTCCTCGCCCGCCC
GCCCGCAGCCGAGGAGCCGAGGCCGCCGGGGCCGTGGCGGCGGAGCCCTCAGCCATGGCCTCGGGCGACA
CCCTCTACATCGCCACGGACGGCTCGGAGATGCCGGCCGAGATCGTGGAGCTGCACGAGATCGAGGTGGA
GACCATCCCGGTGGAGACCATCGAGACCACAGTGGTGGGCGAGGAGGAGGAGGAGGACGACGACGACGAG
GACGGCGGCGGTGGCGACCACGGCGGCGGGGGCGGCCACGGGCACGCCGGCCACCACCACCACCACCATC
ACCACCACCACCACCCGCCCATGATCGOTCTGCAGCCGCTGGTCACCGACGACCCGACCCAGGTGCACCA
CCACCAGGAGGTGATCCTGGTGCAGACGCGCGAGGAGGTGGTGGGCGGCGACGACTCGGACGGGCTGCGC
GCCGAGGACGGCTTCGAGGATCAGATTCTCATCCCGGTGCCCGCGCCGGCCGGCGGCGACGACGACTACA
TTGAACAAACGCTGGTCACCGTGGCGGCGGCCGGCAAGAGCGGCGGCGGCGGCTCGTCGTCGTCGGGAGG
CGGCCGCGTCAAGAAGGGCGGCGGCAAGAAGAGCGGCAAGAAGAGTTACCTCAGCGGCGGGGCCGGCGCG
GCGGGCGGCGGCGGCGCCGACCCGGGCAACAAGAAGTGGGAGCAGAAGCAGGTGCAGATCAAGACCCTGG
AGGGCGAGTTCTCGGTCACCATGTGGTCCTCAGATGAAAAAAAAGATATTGACCATGAGACAGTGGTTGA
AGAACAGATCATTGGAGAGAACTCACCTCCTGATTATTCAGAATATATGACAGGAAAGAAACTTCCTCCT
GGAGGAATACCTGGCATTGACCTCTCAGATCCCAAACAACTGGCAGAATTTGCTAGAATGAAGCCAAGAA
AAATTAAAGAAGATGATGCTCCAAGAACAATAGCTTGCCCTCATAAAGGCTGCACAAAGATGTTCAGGGA
TAACTCGGCCATGAGAAAACATCTGCACACCCACGGTCCCAGAGTCCACGTCTGTGCAGAATGTGGCAAA
GCTTTTGTTGAGAGTTCAAAACTAAAACGACACCAACTGGTTCATACTGGAGAGAAGCCCTTTCAGTGCA
CGTTCGAAGGCTGTGGGAAACGCTTTTCACTGGACTTCAATTTGCGCACACATGTGCGAATCCATACCGG
AGACAGGCCCTATGTGTGCCCCTTCGATGGTTGTAATAAGAAGTTTGCTCAGTCAACTAACCTGAAATCT
CACATCTTAACACATGCTAAGGCCAAAAACAACCAGTGAAAAGAAGAGAGAAGACCCTTCTCGACCACGG
NAAGCATCTTCCAGAAGTGTGATTGGGAATAAATATGCCTCTCCTTTGTATATTATTTCTAGGAAGAATT
TTAAAAATGAATCCTACACACCTAAGGGACATGTTTTGATAAAGTAGTAAAAAAAAAAAAAAAAAAACTT
TACTAAGATGACATTGCTAAGATGCTCTATCTTGCTCTGTAATCTCGTTTCAAAAACACAGTGTTTTTGT
AAAGTGTGGTCCCAACAGGAGGACAATTCATGAACTTCGCATCAAAAGACAATTCTTTATACAACAGTGC
TAAAAATGGGACTTCTTTTCACATTCTTATAAATATGAAGCTCACCTGTTGCTTACAATTTTTTTAATTT
TGTATTTTCCAAGTGTGCATATTGTACACTTTTTTGGGGATATGCTTAGTAATGCTACGTGTGATTTTTC
TGGAGGTTGATAACTTTGCTTGCAGTAGATTTTCTTTAAAAGAATGGGCAGTTACATGCATACTTCAAAA
GTATTTTCCTGTAAAAAAAAAAAAAGTTATATAGGTTTTGTTTGCTATCTTAATTTTGGTTGTATTCTTT
GATGTTAACACATTTTGTATAATTGTATCGTATAGCTGTATTGAATCATGTAGTATCAAATATTAGATGT
GATTTAATAGTGTTAATCAATTTAAACCCATTTTAGTCACTTTTTTTTTCCAAAAAAATACTGCCAGATG
CTGATGTTCAGTGTAATTTCTTTGCCTGTTCAGTTACAGAAAGTGGTGCTCAGTTGTAGAATGTATTGTA
CCTTTTAACACCTGATGTGTACATCCCATGTAACAGAAAGGGCAACAATAAAATAGCAATCCTAAAG
CEX1
SEQ ID NO:66
>gi | 773207 | emb | X77824.1 | HSM3 H. sapiens chromobox 2 gene
CTGCCAGAGATGAGTGCAGGTGAGGAGAGTAGCAGCTCGGACTCCGACCCCGACTCCGCCTCGCCGCCCA
GCACTGGACAGAACCCATCAGTGTCCGTTCAGACCAGCCAGGACTGGAAGCCCACCCGCAGCCTCATCGA
GCACGTATTTGTCACCGACGTCACTGCCAACCTCATCACCGTCACAGTGAAGGAGTCTCCCACCAGCGTC
GGCTTCTTCAACCTGAGGCATTACTGAAGCCCCGGCGCCACCAGCTGCGCGTCTTACTCCCCTTCCCTGC
CTATGGTGTCGCTTGGCTAAGTGACTCCCAGCCCAAGCCCCCTCAAGAGTCTGATCGTCGGGGGAGGAGG
AGTGGG
CEX6
SEQ ID NO:67
Attatgggctgtgggtgccgctgagcaagatggagctgtctgcagtgggcgagcgggtct
Tcgcggccgaatccatcatcaaacggcggatccgaaagggacgcatcgagtacctggtga
Aatggaaggggtgggcgatcaagtacagcacttgggagcccgaggagaacatcctggact
Cgcggctcattgcagccttcgaacaaaaggagagggagcgtgagctgtatgggcccaaga
Agaggggacccaaacccaaaactttcctcctgaaggcgcgggcccaggccgaggccctcc
Gcatcagtgatgtgcatttctctgtcaagccgagcgccagtgcctcctcgcccaagctgc
Actccagcgcagccgtgcaccggctcaagaaggacatccgccgctgccaccgtatgtccc
Gccgtcccctgccccgcccggacccgcaggggggcagccccggactgcgcccgcccattt
Cgcccttctcggagacggt cgcatcatcaaccgcaaggtgaagccgcgggagcccaagc
Ggaaccgcatcatcctgaacctgaaggtgatcgacaagggcgctggcggcgggggcgccg
Ggcagggggccggggcgctggcccgccccaaagtcccctcgcggaaccgcgttataggca
Agagcaagaagttcagcgagagcgtcctgcgtacacagatccgccacatgaagttcggcg
Cctttgcgctgtacaagcctccgcccgcccccctggtagccccgtcccccggcaaggctg
Aggcctcagccccgggccctgggctacttctggccgcccccgccgccccctacgacgccc
Gcagctctggctcctccggctgcccctcgcctacaccacagtcctctgaccccgacgaca
Cgccccccaagctcctccccgagaccgtgagcccatccgcccccagctggcgcgagccgg
Aggtgctcgacctgtccctccctcccgagtcggcagccaccagcaagcgggcaccgcctg
HPC2 (CBX4)
SEQ ID NO:68
>gi | 4503032 | ref | NM_001880.1 | Homo sapiens activating transcription
factor 2 (ATF2), mRNA
GAATTCTGTGATAAGTTATTCAACTTATGAAATTCAAGTTACATGTGAATTCTGCCAGGCAATACAAGGA
CCTGTGGAATATGAGTGATGACAAACCCTTTCTATGTACTGCGCCTGGATGTGGCCAGCGTTTTACCAAC
GAGGATCATTTGGCTGTCCATAAACATAAACATGAGATGACACTGAAATTTGGTCCAGCACGTAATGACA
GTGTCATTGTGGCTGATCAGACCCCAACACCAACAAGATTCTTGAAAAACTGTGAAGAAGTGGGTTTGTT
TAATGAGTTGGCGAGTCCATTTGAGAATGAATTCAAGAAAGCTTCAGAAGATGACATTAAAAAAATGCCT
CTAGATTTATCCCCTCTTGCAACACCTATCATAAGAAGCAAAATTGAGGAGCCTTCTGTTGTAGAAACAA
CTCACCAGGATAGTCCTTTACCTCACCCAGAGTCTACTACCAGTGATGAGAAGGAAGTACCATTGGCACA
AACTGCACAGCCCACATCAGCTATTGTTCGTCCAGCATCATTACAGGTTCCCAATGTGCTGCTTACAAGT
TCTGACTCAAGTGTAATTATTCAGCAGGCAGTACCTTCACCAACCTCAAGTACTGTAATCACCCAGGCAC
CATCCTCTAACAGGCCAATTGTCCCTGTACCAGGCCCATTTCCTCTTCTGTTACATCTTCCTAGTGGACA
AACCATGCCTGTTGCTATTCCTGCATCAATTACAAGTTCTAATGTGCATGTTCCAGCTGCAGTCCCACTC
GTTCGACCAGTCACCATGGTGCCTAGTGTTCCAGGAATCCCAGGTCCTTCCTCTCCCCAACCAGTACAGT
CAGAAGCAAAAATGAGATTAAAAGCTGCTTTGACCCAGCAACATCCTCCAGTTACCAATGGTGATACTGT
CAAAGGTCATGGTAGCGGATTGGTTAGGACTCAGTCAGAGGAATCTCGACCGCAGTCATTACAACAGCCA
GCCACATCCACTACAGAAACTCCGGCTTCTCCAGCTCACACAACTCCACAGACCCAAAGTACAAGTGGTC
GTCGGAGAAGAGCAGCTAACGAAGATCCTGATGAAAAAAGGAGAAAGTTTTTAGAGCGAAATAGAGCAGC
AGCTTCAAGATGCCGACAAAAAAGGAAAGTCTGGGTTCAGTCTTTAGAGAAGAAAGCTGAAGACTTGAGT
TCATTAAATGGTCAGCTGCAGAGTGAAGTCACCCTGCTGAGAAATGAAGTGGCACAGCTGAAACAGCTTC
TTCTGGCTCATAAAGATTGCCCTGTAACCGCCATGCAGAAGAAATCTGGCTATCATACTGCTGATAAAGA
TGATAGTTCAGAAGACATTTCAGTGCCGAGTAGTCCACATACGGAAGCTATACAGCATAGTTCGGTCAGC
ACATCCAATGGAGTCAGTTCAACCTCCAAGGCAGAAGCTGTAGCCACTTCAGTCCTCACCCAGATGGCGG
ACCAGAGTACAGAGCCTGCTCTTTCACAGATCGTTATGGCTCCTTCCTCCCAGTCACAGCCTTCAGGAAG
TTGATTAAAAACCTGCAGTACAACAGTTTAGATACTCATTAGTGACTTCAAAGGGAAATCAAGGAAAGAC
CAGTTTCCATTTATGCGAAATCTGTGGTTGTAAATTT
HPC3
SEQ ID NO:69
>gi | 10190681 | ref | NM_020649.1 | Homo sapiens chromobox homolog 8 (Pc class
homolog, Drosophila) (CBX8), mRNA
CTGACGTCAGCGGGAGAGTATTATGGTCTGTCGTGCGCTGGTGCTGCTTTTCTGCTCCTGGACAGCGGCC
TAGGGGGGAAGCGGCGAGTCAACATGGAGCTTTCAGCGGTGGGGGAGCGGGTGTTCGCGGCCGAAGCCCT
CCTGAAGCGGCGCATACGGAAAGGACGCATGGAATACCTCGTGAAATGGAAGGGATGGTCGCAGAAGTAC
AGCACATGGGAACCGGAGGAAAACATCCTGGATGCTCGCTTGCTCGCAGCCTTTGAGGAAAGGGAAAGAG
AGATGGAGCTCTATGGCCCCAAAAAGCGTTACCCAAGCCCAAAACCTTCCTCCTCAAAGCGCAGGGCCAA
GGCAAAGGCCAAAACTTACGAGTTTCGAAGTGACTCAGCCAGGGGCATCCGGATCCCCTACCCTGGCCGC
TCGCCCCAGGACCTGGCCTCCACTTCCCGGGCCCGGGAGGGCCTTCGAAACATGGGTTTGTCCCCGCCAG
CGAGCAGCACCAGCACCAGCAGCACCTGCCGCGCAGAGGCCCCTCGGGACCGGGACCGAGACCGGGATAG
GGACCGGGAGCGGGATCGAGAAAGGGAGAGGGAGCGAGAGAGGGAGCGGGAACGTGAGAGGGAACGAGAG
CGGGGTACCAGCAGAGTGGATGACAAGCCCAGCTCACCGGGGGACAGTCGAAGAAGTCGAGGCCCCAAGC
CCCGGAAGGAGCTCCCGGACCCCTCACAGAGGCCCTTAGGCGAACCCAGCGCCGGCCTCGGAGAGTACCT
CAAGGGCAGGAAGCTGGACGACACCCCTTCCGGGGCAGGAAAGTTTCCAGCCGGCCACAGTGTGATCCAG
CTGGCCCGAAGACAGGACTCGGACCTGGTGCAGTGTGGTGTGACCAGCCCTAGCTCAGCTGAGGCCACGG
GCAAACTGGCTGTGGACACCTTCCCGGCCAGGGTGATAAAGCACAGGGCTGCCTTCCTGGAGGCCAAAGG
CCAGGGTGCCCTAGATCCCAATGGCACCCGGGTCCGACATGGCTCAGGCCCCCCCAGCTCTGGGGGGGGC
CTGTACCGGGACATGGGGGCCCAGGGGGGAAGGCCCTCCCTCATCGCCAGGATCCCTGTGGCCAGAATCC
TGGGGGACCCGGAGGAAGAGTCCTGGAGCCCCTCCCTGACTAACCTGGAGAAGGTGGTGGTCACGGACGT
GACCTCAAACTTTTTGACCGTCACCATTAAGGAAAGTAACACGGACCAAGGCTTTTTTAAAGAGAAAAGA
TGAATGCTGGGTGGGTGTGTCCAGGAGGAGAGCAGGGGAGAGAGTGAGCGTGAGCTTGGCATAGTGATTT
TTATTTCTGGGTGGGATGTGGCCTTTTGGCTGGTCCCGTCCCTGATGTCACCCCCACCCCACCAGCCCCT
TTCATCCCTCCTTCCTCCCCCTCAGTTTTTGTTGGAAAGATTATCTCTAGAGTTATATTTTCTATTAGAT
GTAAATATGTTATTTAAGAAAAATATCTAAATATATATATTTCAACTCGAAAAAAAAAAAAAAAAACTCG
AG
PHF1
SEQ ID NO:70
>gi | 13435396 | ref | NM_024165.1 | Homo sapiens PHD finger protein 1 (PHF1),
transcript variant 2, mRNA
CTCCTCCTGCCGCTGCCGCTGCTTTGGCTGCTGCGTCATACGCCCCAGAGCCGCCGGGACGGAGGGGCTG
GGCCTGGGGACCCCCCGGCCTCCGCCTGCACGCCCCCCCACGCCCGGACGTGCCCTCTCCGCGCGGGGGA
CTCGCCTAGGTCTCCTACGTCTGCCCCTGCCCGGCTCCCGGCGGCCCCAGCTGTCACCGGCCCCCCCAGG
ATGCAATGGCGCAGCCCCCCCGGCTGAGCCGCTCTGGTGCCTCCTCACTTTGGGACCCAGCTTCTCCTGC
TCCCACCTCTGGCCCCAGGCCTCGGCTTTGGGAGGGTCAAGATGTGCTGGCCAGATGGACTGATGGGCTG
CTATACTTGGGTACCATCAAAAAGGTGGACAGTGCTAGGGAGGTGTGTCTGGTCCAGTTTGAGGATGATT
CGCAGTTTCTGGTTCTATGGAAAGACATTAGCCCTGCTGCCCTCCCTGGAGAGGAACTCCTCTGTTGTGT
CTGTCGCTCTGAGACTGTGGTCCCTGGGAACCGGCTGGTCAGCTGTGAGAAGTGTCGCCATGCTTATCAC
CAGGACTGCCATGTTCCCAGGGCTCCAGCCCCTGAGAGGGAGAGGGCACATCCTGGGTATGGCCGCCAGT
GTGTCTTTGCGATCGCCACCAAGAGGGGAGGTGCCCTGAAGAAGGGCCCCTATGCCCGGGCCATGCTGGG
TATGAAGCTTTCCTGCCATATGGACTGAAGGGGCTGGACTGGGATGCTGAGACATCTGAGCAACCGACAG
CAGAGTTACTGTTACTGTGGTGGCCCTGGGGAGTGGAACCTGAAAATGCTGCAGTGCCGGAGCTGCCTGC
AGTGGTTCCATGAGGCCTGCACCCAGTGTCTGAGCAAGCCCCTCCTCTATGGGGACAGGTTCTATGAATT
TGATGCTGTGTGTGTCGCGGGGGCCCTGAGAAAGTCCGGAGACTACAGCTTTCGCTGGGTGGATGTGGCC
CATCTTGTCCTGTATCACCTCAGTGTTTGCTGTAAGAAGAAATACTTTGATTTTGATCGTGAGATCCTCC
CCTTCACTTCTGAGAATTGGGACAGTTTGCTCCTGGGGGAGCTTTCAGACACCCCCAAAGGAGAACGTTC
TTCCAAGCTCCTCTCTGCTCTTAACAGCCACATTGGACCGTTTCATTTCAGGGAGAGAGATAAGAAGAGG
AAATGTTTGTTTGGTCTCCATGCTCGGATGCCTCCCCCTGTGGAGCCCCCTACTGGAGATGGAGCACTCA
CCAGCTTCCCTTCAGGGCAGGGCCCTGGGGGAGGGGTCTCACGTCCCCTGGGGAAGCGCCGGAGGCCGGA
GCCAGAGCCCCTGAGGAGGAGGCAGAAGGGGAAAGTGGAGGAGCTGGGGCCACCCTCAGCAGTGCGCAAT
CAGCCCGAGCCCCAGGAGCAGAGGGAGCGGGCTCATCTGCAGAGGGCACTGCAGGCCTCAGTGTCTCCAC
CATCCCCCAGCCCTAACCAGAGTTACCAGGGCAGCAGCGGCTACAACTTCCGGCCCACAGATGCCCGCTG
CCTGCCCAGCAGCCCCATCCGGATGTTTGCTTCCTTCCACCCTTCTGCCAGCACCGCAGGGACCTCTGGG
GACAGTGGACCCCCAGACAGGTCACCCCTGGAACTTCACATTGGTTTCCCCACAGACATCCCTAAAAGTG
CCCCCCACTCGATGACTGCCTCATCTTCCTCAGTTTCATCCCCATCCCCAGGTCTTCCTAGACGCTCAGC
ACCCCCTTCTCCCCTGTGCCGTAGTTTGTCTCCTGGGACTGGGGGAGGAGTCCGAGGTGGGGTTGGTTAC
CTGTCCCGAGGGGACCCTGTCCGGGTCCTTGCTCGGAGAGTACGGCCTGATGGCTCTGTGCAGTACCTGG
TTGAGTGGGGAGGAGGGGGCATCTTCTGAACAGCCTGCCTCTGCCCAGCTCCCCATTCACACACACCGGC
ACTTTCATACCCTGACCTCTGACCTCACCTACAGCTGGGATGTACCTGGAGAGATAGGGGGTAGTTCTCC
CTACTGCCCAGGCTGGAATCCAAGAGTGGGGAGTGGGGAAGAGGCCCTCTTCTCTACCCTCCTTCATGAT
TCCTGACCCCTCCCATCCTTCCCATTTCCTTTGATGTTATTTTGTTACAGCTTTTTAAATATTTTTTAAA
ATTATTTAACCCCTGGGGGCAGAGACTGAGGAGGGAGGATGATAAGGGATCCCGGACTCTGTATGATTGA
ATAAAGAGAAATAAACAAA
PHF2
SEQ ID NO:71
>gi | 4885546 | ref | NM_005392.1 | Homo sapiens PHD finger protein 2 (PHF2), mRNA
GCGGCCGCTCGGCGGCCGGGGGTCCCTTCGGTGGGGCCGCGGCTCCCCGCCCGCCGCCCCCGCGCGTCCA
TTCGCTTTGTGTCCCGCGCGCGGCCGGGCCCCCCGCGCACTCTCAGCCCTGCGCCCCGCGGCCCGGCGGG
CGGCTCCCGGCGCGGCCCCAGCAGCCCGCGCCGGCATTGTGTGGACGCGCCCGGCCGCGAGCGCGCGCGC
GGGCCCTGCCGAGCGCCCCCGGCCCCGTCCGCTCCGGCCGCGGCGCCCGCGCCCGCCGCCCCCGCCGCCC
TCGCCGCGCGGCCCCCGGCCCGGCCCGGCCCGACCCGGGCAGCGCAGCGGOGGGGCGAGCGGCGGCGCGG
CAACATGGCGACGGTGCCCGTGTACTGCGTCTGCCGGCTGCCCTACGACGTTACCCGCTTTATGATCGAG
TGCGATGCCTGCAAGGACTGGTTCCACGGCAGCTGTGTTGGGGTGGAAGAGGAAGAGGCACCAGACATCG
ACATTTACCACTGCCCGAACTGCGAGAAAACCCATGGCAAGTCCACACTCAAGAAAAAGCGGACTTGGCA
CAAACACGGCCCTGGGCCAACACCGGACGTGAAACCAGTGCAGAATGGCAGTCAGCTGTTCATCAAGGAG
CTGCGGAGCCGAACCTTCCCCAGTGCTGAAGACGTGGTGTCCCGTGTGCCAGGTAGCCAGCTCACCGTGG
GCTACATGGAGGAGCATGGCTTCACTGAGCCCATCCTTGTCCCCAAGAAAGATGGCCTGGGCTTAGCTGT
CCcTGCCCCAACATTCTACGTGAGTGACGTCGAGAACTACGTGGGGCCGGAACGGAGTGTGGATGTGACA
GATGTCACCAAGCAGATGGACTGCAAGATGAAGCTGAAGGAGTTTGTGGACTATTACTACAGCACCAACC
GCAAGCGGGTCCTCAACGTCACCAACCTTCGAGTCTCTGACACCCGAATGTCCAGCTTCGTGGAGCCACC
TGACATTGTAAAGAAACTGTCATGGGTAGAAAAcTACTGGCCAGATGATGCATTGCTGGCCAAGCCCAAA
GTGACCAAGTACTGCCTAATCTGCGTGAAGGACAGTTACACCGACTTCCACATCGACTCTGGGGGCGCCT
CTGCCTGGTACCACGTGCTCAAGGGGGAGAAGACCTTCTATCTCATCAGGCCGGCCTCGGCCAACATCTC
CCTGTATGAGCGCTGGCGGTCTGCCTCTAACCACAGCGAGATGTTCTTTGCTGACCAGGTCGACAAATGC
TACAAGTGCATCGTCAAGCAGGGCCAGACCCTCTTCATCCCCTCAGGCTGGATCTACGCCACACTCACCC
CTGTGGACTGCCTGGCCTTCGCGGGACATTTCCTCCACAGCCTGAGTGTGGAGATGCAGATGAGAGCATA
CGAGGTGGAAAGGAGGTTGAAACTTGGCAGCCTGACTCAGTTTCCCAACTTTGAAACTGCGTGCTGGTAC
ATGGGAAAGCACCTATTGGAGGCGTTCAAAGGTTCTCACAAGTCTGGGAAGCAGCTGCCCCCACATCTAG
TCCAAGGAGCTAAAATTCTCAATGGTGCTTTCCGATCGTGGACGAAGAAGCAGGCTTTGGCAGAGCATGA
GGACGAGCTCCCGGAGCACTTCAAACCTTCACAGCTAATCAAGGACCTGGCCAAAGAGATCCGGCTCAGT
GAGAATGCCTCCAAAGCCGTCCGACCGGAAGTGAATACTGTCGCCTCGTCAGATGAGGTGTGTGACGGGG
ACCGGGAGAAGGAGGAGCCCCCGTCTCCCATTGAGGCCACCCCGCCTCAATCCCTCCTGGAGAAAGTGTC
CAAAAAAAAGACTCCCAAAACTGTGAAGATGCCCAAGCCATCCAAAATCCCCAAGCCCCCGAAGCCCCCT
AAGCCCCCAAGGCCCCCCAAAACGCTGAAGCTCAAAGATGGAGGCAAGAAGAAAGGGAAGAAGTCCCGGG
GATTCTGCGGTGTACAAGAGCGATGACTCCTCTGACGAGGGCTCTCTGCACATCGACACGGACACCAAGC
CAGGCAGAAATGCCAAAGTGAAGAAGGAGAGTGGGAGCTCCGCGGCCGGCATCCTGGACCTGCTGCAGGC
CAGCGAGGAGGTTGGCGCACTCGAGTACAACCCCAACAGCCAGCCCCCTGCCTCCCCCAGCACACAGGAA
GCCATTCAGGGAATGCTCTCCATGGCCAATCTGCAGGCCTCTGACTCTTGCCTGCAGACCACATGGGGCA
CGGGGCAGGCCAAGGGTGGCTCACTGGCAGCCCATGGTGCCCGGAAGATTGGTGGTGGCAACAAAGGCAC
AGGCAAGCGCCTGCTGAAGAGGACTGCCAAGAACAGTGTGGATCTGGAGGACTACGAGGAGCAGGATCAC
CTGGATGCCTGCTTCAAGGACTCAGACTATGTTTACCCCTCACTGGAGTCTGACGAAGATAACCCCGTCT
TCAAGTCCCGGTCAAAGAAGAGGAAAGGCTCAGACGATGCTCCGTACAGCCCCACAGCCAGGGTCGGTCC
CGTACAAAAAAGGGCATGGCCACCGCCAAGCAAAGGCTTGGAAAGATCTTGAAGATCCATCGQAATGGGA
AACTGCTCCTCTAAGGCTTGGTAAGCCAGGATCCTTCTGATATGCTAAGGACCCCCGGAGCCCCGCTACA
TCAGCCCCTCCCAGGACGGTGGcTGTGCCGCCTGGCCCGGGGAGGGCTTGCTTCATTCCGACCAATTTTC
CAATCAA
HPH1
SEQ ID NO:72
>gi | 11038623 | ref | NM_004426.1 | Homo sapiens early development regulator 1
(polyhomeotic 1 homolog) (EDR1), mRNA
CCCGCCTCGGCGCCCCCGCCCTCCAGAAAGGGGAGGAGGCGAGGGGAGCCCGCCGCGGAGGCCGAGAGCG
AGCCGGCGCCCAGCCCAGCCTGGCGACTGGGGACCCCGGCACATGAGGTGGACGCCCCGGGGAAGACTTG
GGTGCACAGCCAGGCGAGAAGGTCTTGAGTCAGACAGAGCACCAGCCTTGGGGACCCTGGACCACTATCA
TGGAGACTGAGAGCGAGCAGAACTCCAATTCCACCAATGGGAGTTCTAGCTCAGGGGGCAGCTCTCGGCC
CCAGATAGCTCAAATGTCACTTTATGAACGACAAGCAGTGCAGGCTCTGCAAGCACTGCAGCGGCAGCCC
AATGCAGCTCAGTATTTCCACCAGTTCATGCTCCAGCAGCAGCTCAGTAATGCCCAGCTGCATAGCCTGG
CTGCCGTCCAGCAGGCCACAATGCTGCCAGTCGGCAGGCCAGCTCACCCAAACACCAGCACTACACAGCA
GCAGACTACCACCACCCAGGCCTCGATCAATCTGGCCACCACATCGGCCGCCCAGCTCATCAGCCGATCC
CAGAGTGTGAGCTCTCCCAGTGCTACCACCTTGACCCAATCTGTGCTACTGGGGAACACCACCTCCCCAC
CCCTcACCAGTCTCAGGCCCAGATGTATCTACGGCCACAGCTGGGAAACCTATTTGCAGGTAAACCGAAC
CCCTGGGTCGAATGTGCCTCTAGCCTCCCAACTCATCCTGATGCCTAATGGGGCGGTGGCTGCAGTCCAG
CAGGAGGTGCCATCTGCTCAGTCTCCGGAGTTCATGCACGATGCAGATCAGGTTCAGAACTTGGCAGTAA
GGAATCAACAGGCCTCAGCTCAAGGACCTCAGATGCAAGGCTCCACTCAGAAGGCCATTCCTCCAGGAGC
CTCCCCTGTCTCTAGCCTCTCCCAGGCCTCTAGCCAGGCCCTAGCGGTGGCACAGGCTTCCTCTGGGGCC
ACAAACCAGTCCCTCAACCTTAGTCAAGCTGGTGGAGGCAGTGGGAATAGCATCCCAGGGTCCATGGGTC
CAGGTGGAGGTGGGCAGGCACATGGTGGTTTGGGTCAGTTGCCTTCCTCAGGAATGGGTGGTGGGAGCTG
TCCCAGGAGGGTACAGGAGTGGTGCAAGCCCTTGCCTGCAGCCCAAACAGTAACTGTGAGCCAGGGCAGC
CAGACAGAGGCAGAAAGTGCAGCAGCCAAGAAGGCAGAAGCAGATGGGAGTGGCCAGCAGAATGTGGGCA
TGAACCTGACACGGACAGCCACACCTGCGCCCAGCCAGACACTTATTAGCTCAGCCACCTACACACAGAT
CCAGCCCCATTCACTGATTCAGCAACAGCAACAGATCCACCTCCAGCAGAAACAGGTGGTGATCCAGCAG
CAGATTGCCATCCACCACCAGCAGCAGTTCCAGCACCGGCAGTCCCAGCTCCTTCACACAGCTACACACC
TCCAGTTGGCGCAGCAGCAGCAGCAGCAACAACAGCAACAGCAGCAACAGCAGCAGCCGCAAGCCACCAC
CCTCACTGCCCCTCAGCCACCACAGGTCCCACCTACTCAGCAGGTCCCACCTTCCCAGTCCCAGCAGCAA
GCCCAAACCCTGGTCGTTCAGCCCATGCTTCAGTCTTCACCCTTGTCTCTTCCACCTGATGCAGCCCCTA
AGCCACCAATTCCCATCCAATCCAAACCACCTGTAGCACCTATCAAGCCGCCTCAGTTAGGGGCCGCTAA
GATGTCAGCTGCCCAGCAACCACCACCCCATATCCCTGTGCAAGTTGTAGGCACTCGACAGCCAGGTACA
GCCCAGGCACAGGCTTTGGGGTTGGCACAGCTGGCAGCTGCTGTACcTACTTCCCGGGGGATGCCAGGTA
CAGTGCAGTCTGGTCAGCCCCTAAAGGCTTCCTCGCCACCTTCATCCCAGGCTCCTGGTGCACTGCAGGA
GTGCCCTCCCACATTGGCCCCGGGGATGACCCTTGCTCCTGTGCAGGGGACAGCACATGTGGTAAAGGGT
GGGGCTACCACCTCCACACCTGTTGTAGCCCAGGTCCCTGCTGCCTTOTATATGCAGTCTGTGCACTTGC
CGGGAAACCCCAGACATTGGCTGTCAAACGCAAGGCTGACTCTGAGGAGGAGAGAGATGATCGTCTCCAC
ATTGGGTTCAATGCTTCCTGCCAAGGCATCTCCAGTAGCAGAAAGCCCAAAAGTCATGGACGAGAAGAGC
AGTCTTGGAGAAAAAGCTGAATCAGTGGCTAATGTGAATGCTAATACTCCAAGCAGTGAACTAGTAGCCT
TGACCCCCGCCCCTTCAGTACCGCCTCCTACACTAGCCATGGTGTCTAGACAAATGGGTGACTCAAAACC
CCCACAGGCCATCGTGAAGCCCCAGATTCTCACCCACATCATTGAAGGCTTTGTTATCCAGGAAGGAGCA
GAACCTTTCCCCGGTGGTTGTTCTCAGTTACTGAAGGAGTCTGAGAAGCCACTACAGACTGGCCTTCCGA
CAGGCTTGACTGAGAATCAGTCAGGTGGCCCCTTTGGAGTGGACAGCCCATCTGCTGAGTTAGATAAGAA
GGCGAATCTCCTGAAGTGCGAGTACTGTGGGAAGTACGCCCCCGCAGAGCAGTTTCGTGGCTCTAAGAGG
TTCTGCTCCATGACCTGCGCTAAGAGGTACAATGTGAGCTGTAGCCATCAGTTCCGGCTGAAGAGGAAAA
AAATGAAAGAGTTTCAAGAAGCCAACTATGCTCGCGTTCGCAGGCGTGGACCCCGCCGCAGCTCCTCTGA
CATTGCCCGTGCCAAGATTCAGGGCAAGTGCCACCGGGGTCAAGAAGACTCTAGCCGGGGTTCAGATAAT
TCCAGTTATGATGAAGCACTCTCTCCAACATCTCCTGGGCCTTTATCAGTAAGAGCTGGGCATGGAGAAC
GTGACCTGGGGAATCCCAATACAGCTCCACCTACACCGGAATTACATGGCATCAACCCTGTGTTCCTGTC
CAGTAATCCCAGCCGTTCGAGTGTAGAGGAGGTCTACGAGTTTATTGCTTCTCTCCAAGGCTGCCAAGAG
ATTGCAGAGGAATTTCGOTCACAGGAGATTGATGGACAGGCCTTTTTATTACTTAAAGAAGAACATCTTA
TGAGTGCCATGAACATCAAGCTGGGCCCTGCCCTCAAGATCTGTGCCAAGATCAATGTCCTCAAGGAGAC
CTAAGGTGGCCCTCTTGCACAAACCAGCCTAAGGCAGACAcTCTCCACTGTCCAGGTTATAACCTGGTAC
CAGCAGACTTTGCAGGGAAGAAAGAGTTGTTCCAATCATGTAACCTTCTGTAGGGGATTACTGAGACAGG
GAAGAGAAGTGCAAGAATTGGTTGCTGGTGCTACATGGCGGCAGCTTTGACATTTTCTCTGGGTTCTACT
TTATTTTTTAAAATCTTTACAGTTCTCACCATTTCACGTACCTTAATCCAATCTTTATAAAAGAGGCAGT
CTAGAGAAATAGGACTGCTCAGCCTTATCCTGGAGTGGAGCATTTAGCCCAGGTCTTAATTCTCCAAGAG
GAGGAATACATAGTATGGTAAGGCAAGGAACTGGGTAGAATGTCAGGTTGCCTGCCCAATGGGAGAGGTA
GGGTTTTTCTAGCTTGTGTGACAGAAGTAGCAAAATCTGGTCCTCCCCCCTCCCAGTGTAGCTGTGGCTC
AGAGTTTTTTCTTTTTGTTGTCACTTACTCCCTTGTGATTGAATTTTTTCTCCTGCATCCATGGCAGGAT
CCCCAGCCAGTATAGAGACTTGGTTGGCATCTTCTGCTGCAGGGACTAAAAGTATTTGACTGGGGCACAT
GTGGOTGTTGTCATTCTTTCTGCATCCCACTGTTCCCCTCCAATTTATGTTATTTTCTACCCTGTTTTTC
AGTTCCATCTCTGCTCTGTCCTATAGCTTTATAAAACCAGAGTGTGTGGGGCTGAGGTCAGGAGTATAAG
TACCTGCCTTAGGCACTATTCCTTATATAACAAAAATTAAATATTTTTTTCAGTAAAAGGATG
HPH2
SEQ ID NO:73
>gi | 4758241 | ref | NM_004427.1 | Homo sapiens early development regulator 2
(polyhomeotic 2 homolog) (EDR2), mRNA
GGCGCCGCATGTGTCTCCGCGGCGGCTGCAGCCCTCGAGCGCCCGCCGCCGCGCCCCAACCCCGGCCGCC
GCCCGCCCTCCCGCOCCGGCCTCGCGCCCCCGTCCCGGCCTCGCGCCCCGGCCGCCCTTTGTTGACGCCG
GCCAGGCCGTGCGGTCGGATGCGCCGCGGCAGCCCCGGGCCCCGGOTCGGAGGCTCCCGGGGCGAGAGGA
GGCGGCCCGCCGGCCGGGACCCCGCGCGAGTCGGCCCCGGCCAGGGGCTGCGTAGGCCCGCCCGGCCAGG
CCCAGCCGCCTGGACAGAGACAGGGCAGGGCATTGTTCATGCACTGACCGACCTCAGCATCCCCGGCATG
ACCTCAGGGAACGGAAACTCTGCCTCCAGCATCGCCGGCACTGCCCCCCAGAATGGTGAGAATAAACCAC
CACAGGCCATTGTGAAACCCCAAATCCTGACGCATGTTATCGAAGGGTTTGTGATCCAGGAGGGGGCGGA
CGTTTCCCGGTGGGACGCTCGTCTGTGGTGGGGAATCTCAAGAAGAAGTATGCACAGGGGTTCCTGCCCT
GAGAAACTTCCACAGCAGGATCACACCACCACCACTGACTCGGAGATGGAGGAGCCCTATCTGCAAGAAT
CCAAAGAGGAGGGTGCTCCCCTCAAACTCAAGTGTGAGCTCTGTGGCCGGGTGGACTTTGCCTATAAGTT
CAAGCGTTCCAAGCGCTTCTGTTCCATGGCTTGTGCAAAGAGGTACAACGTGGGATGCACCAAACGGGTG
GGACTTTTCCACTCAGACCGGAGCAAGCTGCAGAAGGCAGGAGCTGCGACCCACAACCGCCGTCGGCCAG
CAAAGCCAGTCTGCCACCACTTACCAAGGATACCAAGAAGCAGCCAACAGGCACTGTGCCCCTTTCGGTT
ACTGCTGCTTTGCGTAACACACAGCCAGGAAGACTCCAGCCGTTGCTCAGATAACTCAAGCTATGAGGAA
CCCTTGTCACCCATCTCAGCCAGCTCATCTACTTCCGCCGGCGACAAGGCCAGCGGGACCTGGAGCTCCC
CGACATGCATATGCGGGACCTGGTGGGCATGGGACACCACTTCCTGCCAAGTGAGCCACCAAGTGAATGT
AGAAGACGTCTACGAATTCATCCGCTCTCTGCCAGGCTGCCAGGAGATAGCAGAGGAATTCCGTGCCCAG
GAAATCGACGGGCAAGCCCTGTGCTGCTCAAGGAGGACCACCTGATGAGCGTTATGAACATCAACGCTGG
GGCCCGCCCTGAAGATCTACGCCCGCATCAGCATGCTCAAGGACTCCTAGGGCTGGTGGCACCAGGATTC
TGGCCCAGGGCGCCTCCTCCCGACTGAGCAGAGCCAGACAGACATTCCTGAGGGGCCCAGAAATGGCGGC
GTTGGAGGGCAGGGGCTCTCCCTAGGGGCATAGCTGGTGAGGAGGTCTGGGCACCTCCTCCATGGCTCTC
AGGGGCCTTTCATTTCTGTGGGAGGGGCAGAGAGGTAGGTGGCACAGAAGATGGGCTTTATTGCTTGTAA
ATATTGATAGCACTGGCTTCCTCCAAAGTCCCAATACTCTAGCCCCGCTCTCTTCCCCTCTTTCTGTCCC
CCATTTTCCAGGGGGTATATGGTCAGGGCTCCCCAACCTGAGTTGGTTACTTCAGGGCAGCCAGCCAGGC
CTGGATGGAGGCCTAGAAAGCCCTTGCCTTCCTTCCTCCCACTTCTTTCTCCAGGCCTGGTTAACTCTTC
CGTTGTCAGCTTCTCCCCCTTCAGCCTGTTTCTGCAGCAGCCAGGGTTCTCCCCCCTACACCCTCTGCAG
GTGGAGAGAGAGAAGCTGGGCCCAGCCGCGGTGCCTGCTGGCCAAGACGCCTTAACGCTGTGTGTATGAC
TGTGTGACTGTGTGGGAGCCTGGACTGACAGATAGGCCAAGGGTACTCTCTCGGCATCTCCAGGTGTTTT
GTAGCAAACAGCCACTTAGTGCTTTGTCCTGGACTCCACTCAGCCTCAGGATGGGGAATAGCCAAGAATG
GCAGCCTCAGCGCAGAGGCAAGGTCAGAAAGAGACGGCGCTTCAGAGTTTCCTTTCCAGACACCCCTCCC
CGCACTGTGAAGTTCCCCTGACCGCCCTCCTGGTTCACAAAGAGCATTAAGAAAGCTGCGGTGGTCTGAG
CAACATAGCCCAGACGTGGAGCCTCCTGGCCTGCCTGCCCGCCCACCCTGGGAGTCCAGTGGTGAGGCTC
AGAGAACTTCTAAGGGGAAAGAACAGCTGGAGTTTCTGTTGATGTGAAGAAGGCAGCTCTTGGCCTCCCA
CTCCCACACTTCTTTGCCTATAAATCTTCCTAGCAGCAATTTGAGCTACCTGAGGAGGAGGCAGGGCAGA
AGGGCAAGGGCCTGCCTCTGACCTGCCGTGTCCTTTGCAGGAAGGAGGTAGGCACCTTTCTGAGCTTATT
CTATTCCCCACCCACACCCCCAGGCAGGGTTGGAAATGAAGGACTTTTTTAACCTTTGTTTTGTTTTTTA
AAAATAAATCTGTAAAATCTGAAAAAAAAAAAAAA
SSX1
SEQ ID NO:74
>gi | 5032120 | ref | NM_005635.1 | Homo sapiens synovial sarcoma, X breakpoint 1
(SSX1), mRNA
CACTTTGTCACCAACTGCTGCCAACTCGCCACCACTGCTGCCGCAATCGCAACCACTGCTTTGTCTCTGA
AGTGAGACTGCTCCTGGTGCCATGAACGGAGACGACACCTTTGCAAAGAGACCCAGGGATGATGCTAAAG
CATCAGAGAAGAGAAGCAAGGCCTTTGATGATATTGCCACATACTTCTCTAAGAAAGAGTGGAAAAAGAT
GAAATACTCGGAGAAAATCAGCTATGTGTATATGAAGAGAAACTATAAGGCCATGACTAAACTAGGTTTC
AAAGTCACCCTCCCACCTTTCATGTGTAATAAACAGGCCACAGACTTCCAGGGGAATGATTTTGATAATG
ACCATAACCGCAGGATTCAGGTTGAACATCCTCAGATGACTTTCGGCAGGCTCCACAGAATCATCCCGAA
GATCATGCCCAAGAAGCCAGCAGAGGACGAAAATGATTCGAAGGGAGTGTCAGAAGCATCTGGCCCACAA
GATCATGCCCAAGAAGCCAGCAGAGGACGAAAATGATTCGAAGGGAGTGTCAGAAGCATCTGGCCCACAA
AACGATGGGAAACAACTGCACCCCCCAGGAAAAGCAAATATTTCTGAGAAGATTAATAAGAGATCTGGAC
CCAAAAGGGGGAAACATGCCTGGACCCACAGACTGCGTGAGAGAAAGCAGCTGGTGATTTATGAAGAGAT
CAGTGACCCTGAGGAAGATGACGAGTAACTCCCCTGGGGGATACGACACATGCCCTTGATGAGAAGCAGA
ACGTGGTGACCTTTCACGAACATGGGCATGGCTGCGGCTCCCTCGTCATCAGGTGCATAGCAAGTG
SSX2
SEQ ID NO:75
>gi | 10337582 | ref | Nm_003147.1 | Homo sapiens synovial sarcoma, X breakpoint 2
(SSX2), mRNA
CTCTCTTTCGATTCTTCCATACTCAGAGTACGCACGGTCTGATTTTCTCTTTGGATTCTTCCAAAATCAG
AGTCAGACTGCTCCCGGTGCCATGAACGGAGACGACGCCTTTGCAAGGAGACCCACGGTTGGTGCTCAAA
TACCAGAGAAGATCCAAAAGGCCTTCGATGATATTGCCAAATACTTCTCTAAGGAAGAGTGGGAAAAGAT
GAAAGCCTCGGAGAAAATCTTCTATGTGTATATGAAGAGAAAGTATGAGGCTATGACTAAACTAGGTTTC
AAGGCCACCCTCCCACCTTTCATGTGTAATAAACGGGCCGAAGACTTCCAGGGGAATGATTTGGATAATG
ACCCTAACCGTGGGAATCAGGTTGAACGTCCTCAGATGACTTTCGGCAGGcTCCAGGGAATCTCCCCGAA
GATCATGCCCAAGAAGCCAGCAGAGGAAGGAAATGATTCGGAGGAAGTGCCAGAAGCATCTGGCCCACAA
AATGATGGGAAAGAGCTGTGCCCCCCGGGAAAACCAACTACCTCTGAGAAGATTCACGAGAGATCTGGAC
CCAAAAGGGGGGAACATGCCTGGACCCACAGACTGCGTGAGAGAAAACAGCTGGTGATTTATGAAGAGAT
CAGCGACCCTGAGGAAGATGACGAGTAACTCCCCTCAGGGATACGACACATGCCCATGATGAGAAGCAGA
ACGTGGTGACCTTTCACGAACATGGGCATGGCTGCGGACCCCTCGTCATCAGGTGCATAGCAAGTG

TABLE 3

Enhancers of trithorax and polycomb.

EPC1
SEQ ID NO:76
>gi | 13376809 | ref | NM_025209.1 | Homo sapiens enhancer of polycomb 1 (EPC1),
mRNA
ATGAGTAAACTGTCGTTTCGGGCGCGGGCGCTAGACGCCTCGAAGCCGCTGCCGGTTTTCCGCTGTGAGG
ATCTGCCCGACCTGCACGAATACGCCTCGATAAACAGGGCCGTGCCGCAGATGCCCACCGGAATGGAGAA
GGAAGAGGAGTCGGAACATCATCTTCAGCGGGCTATTTCAGCACAGCAGGTGTATGGCGAGAAGAGGGAT
AATATGGTTATACCGGTCCCAGAGGCAGAAAGTAATATTGCTTACTATGAGTCTATATATCCTGGGGAAT
TTAAGATGCCAAAGCAGCTCATTCACATACAGCCTTTTAGTTTGGATGCTGAACAGCCTGATTATGATTT
GGATTCTGAAGATGAAGTATTTGTGAATAAACTGAAAAAGAAAATGGACATCTGCCCATTGCAATTTGAG
GAGATGATTGACCGCCTAGAAAAAGGCAGTGGTCAGCAGCCAGTCAGTCTGCAGGAAGCCAAACTACTGC
TAAAAGAAGATGATGAACTAATTAGAGAAGTTTATGAATATTGGATTAAAAAGAGAAAAAACTGTCGAGG
GCCATCTCTTATTCCATCAGTAAAACAAGAGAAGCGAGATGGTTCCAGCACAAATGATCCTTATGTGGCT
TTTAGAAGGCGTACTGAAAAAATGCAGACTCGAAAAAATCGCAAAAATGATGAAGCCTCTTACGAAAAAA
TGCTTAAGCTGCGACGAGATCTAAGTCGAGCTGTTACTATTCTAGAGATGATALAAAGAAGAGAAAAAAG
TAAAAGAGAGCTATTGCACTTAACACTGGAATTATGGAAAAAGAGGTATAATTTGGGCGACTACAATGGA
GAGATCATGTCTGAGGTTATGGCACAGAGACAGCCAATGAAACCTACTTATGCCATCCCCATCATCCCTA
TTACTAATAGCAGTCAATTTAAACACCAGGAAGCAATGGATGTGAAGGAGTTCAAAGTTAATAAGCAAGA
TAAAGCCATCTTATCCGACCGAAACGGAAATATGAAAAGAAGCCCAAAGTCTTACCATCGATCTGCCGCT
GCTACTCCCCAACAGACGAGTCCTGCTGCACTGCCAGTCTTCAATGCTAAAGATCTGAATCAGTATGACT
TTCCCAGCTCAGACGAAGAACCTCTCTCCCAGGTTTTGTCTGGCTCTTCGGAAGCTGAGGAAGACAATGA
TCCTGATGGTCCTTTTGCTTTCCGTAGGAAAGCAGGCTGTCAGTACTATGCTCCTCACTTAGACCAAACT
GGCAACTGGCCTTGGACTAGTCCTAAAGATGGAGGAAAAGGGGATGTGCGATATAGATACTGCTTAACTA
CTCTCACCGTACCCCAAAGGTGTATTGGATTTGCACGAAGACGGGTTGGGCGCGGTGGGAAGGGCTTACT
GGACAGAGCTCATTCAGACTATGACAGTGTGTTTCACCATCTGGATTTGGAAATGCTTTCCTCACCACAA
CATTCTCCAGTCAATCAGTTTGCCAATACCTCAGAAACAAATACCTCGGACAAATCTTTCTCTAAAGACC
TCAGTCAGATACTAGTCAATATCAAATCATGTAGATGGCGGCATTTTAGGCCTCGGACACCATCCCTACA
TGACAGTGACAATGATGAACTCTCCTGTAGAAAATTTATATAGGAGTATTACCGAACAGGAACAGCACAA
CCTGGGACCCAGACATGCAGTACCTCTACGCAAAGTAAAAGTAGCAGTGGTTCAGCACACTTTGCATTTA
CAGCCGAACAATACCAGCAACATCAACAGCAACTGGCACTCATGCAGAAACAGCAGCTTGCACAAATTCA
GCAACAGCAAGCAAATAGTAATTCCTCCACCAACACATCACAGAACCTTGCATCTAACCAGCAGAAAAGT
GGCTTTCGCCTGAATATACAGGGTTTAGAAGAACACTACAGGGTTTTGTTTCTTAAGACTTTGGATTCTG
CTAGTGCACAGTTTGCTGCTTCTGCTTTGGTGACATCAGAACAACTGATGGGATTCAAGATGAAGGATGA
TGTGGTGCTTGGAATCGGGGTGAATGGCGTCCTTCCAGCCTCAGGAGTATACAAGAGCTTACACCTCAGT
AGTACTACACCAACAGCACTTGTACATACAAGTCCATCAACGGCAGGTTCAGCTTTGTTACAGCCTTCAA
ATATTACACAGACTTCAAGTTCCCACAGTGCACTGAGTCATCAAGTAACTGCTGCCAATTCTGCAACAAC
TCAGGTTCTGATTGGGAACAACATTCGATTAACTGTACCTTCATCAGTTGCCACTGTAAACTCTATTGCC
CCAATAAATGCACGACATATACCTAGGACTTTAAGTGCTGTTCCATCATCTGCCTTAAAGCTGGCCGCTG
CAGCAAACTGTCAAGTTTCCAAGGTCCCATCTTCATCCTCTGTAGATTCAGTTCCAAGGGAAAATCATGA
ATCAGAAAAGCCAGCACTGAACAACATAGCAGACAACACAGTAGCGATGGAGGTGACGTAG
EZH1
SEQ ID NO:77
>gi | 4503622 | ref | NM_001991.1 | Homo sapiens enhancer of zeste homolog 1
(Drosophila) (EZH1), mRNA
GAGGCTGGACACCTGTTCTGCTGTTGTGTCCTGCCATTCTCCTGAAGAACAGAGGCACACTGTAAAACCC
AACACTTCCCCTTGCATTCTATAAGATTACAGCAGATGAGAAATACCAAATCCCCCTACCTCCAATTGTA
TCACTTACTGGAAAAGAAAAGTGAAATCTGAATACATGCGACTTCGACAACTTAAACGGCTTCAGGCAAA
TATGGGTGCAAAGGCTTTGTATGTGGCAAATTTTGCAAAGGTTCAAGAAAAAACCCAGATCCTCAATGAA
GAATGGAAGAAGCTTCGTGTCCAACGTGTTCAGTCAATGAAGCCTGTGAGTGGACACCCTTTTCTCAAAA
AGTGTACCATAGAGAGCATTTTCCCGGGATTTGCAAGCCAACATATGTTAATGAGGTCACTGAACACAGT
TGCATTGGTTCCCATCATGTATTCCTGGTCCCCTCTCCAACAGAACTTTATGGTAGAAGATGAGAGGGTT
TTGTGCAATATTCCCTACATGGGAGATGAAGTGAAAGAAGAAGATGAGACTTTTATTGAGGAGCTGATCA
ATAACTATGATGGGAAAGTCCATGGTGAAGAAGAGATGATCCCTGGATCCGTTCTGATTAGTGATGCTGT
TTTTCTGGAGTTGGTCGATGCCCTGAATCAGTACTCAGATGAGGAGGAGGAAGGGCACAATGACACCTCA
GATGGAAAGCAGGATGACAGCAAAGAAGATCGCCAGTAACAAGAAAGAGAAAGCAGACATGCTATTGAAG
GCAACAAAAAGAGTTCCAAGAAACAGTTCCCAAATGACATGATCTTCAGTGCAATTGCCTCAATGTTCCC
TGAGAATGGTGTCCCAGATGACATGAAGGAGAGGTATCGAGAACTAACAGAGATGTCAGACCCCAATGCA
CTTCCCCCTCAGTGCACACCCAACATCGATGGCCCCATTGCCAAGTCTGTGCAGCGGGAGCAATCTCTGC
ACTCCTTCCACACACTTTTTTGCCGGCGCTGCTTTAAATACGACTGCTTCCTTCACCCTTTTCATGCCAC
CCCTAATGTATATAACGCAAGAATAAAGAAATCAAGATTGAACCAGAACCATGTGGCACAGACTGCATTC
CTTTTGCTGGAAGGAGCAAAGGAGTATGCCATGCTCCACAACCCCCGCTCCAAGTGCTCTGGTCGTCGCC
GGAGAAGGCACCACATAGTCAGTGCTTCCTGCTCCAATGCCTCAGCCTCTGCTGTGGCTGAGACTAAAGA
AGGAGACAGTGACAGGGACACAGGCAATGACTGGGCCTCCAGTTCTTCAGAGGCTAACTCTCGCTGTCAG
ACTCCCACAAAACAGAAGGCTAGTCCAGCCCCACCTCAACTCTGCGTAGTGGAAGCACCCTCGGAGCCTG
TGGAATGGACTGGGGCTGAAGAATCTCTTTTTCGAGTCTTCCATGGCACCTACTTCAACAACTTCTGTTC
AATAGCCAGGCTTCTGGGGACCAAGACGTGCAAGCAGGTCTTTCAGTTTGCAGTCAAAGAATCACTTATC
CTGAAGCTGCCAACAGATGAGCTCATGTACCCCTCACAGAAGAAGAAAAGAAAGCACAGATTGTGGGCTG
CACACTGCAGGAAGATTCAGCTGAAGAAAGATAACTCTTCCACACAAGTGTACAACTACCAACCCTGCGA
CCACCCAGACCGCCCCTGTGACAGCACCTGCCCCTGCATCATGACTCAGAATTTCTGTGAGAAGTTCTGC
CAGTGCAACCCAGACTGTCAGAATCGTTTCCCTGGCTGTCGCTGTAAGACCCAGTGCAATACCAAGCAAT
GTCCTTGCTATCTGGCAGTGCGAGAATGTGACCCTGACCTGTGTCTCACCTGTGGGGCCTCAGAGCACTG
GGACTGCAAGGTGGTTTCCTGTAAAAACTGCAGCATCCAGCGTGCACTTAAGAAGGACCTGCTGCTGGCC
CCCTCTGATGTGGCCGGATGGGGCACCTTCATAAAGGAGTCTGTGCAGAAGAACGAATTCATTTCTGAAT
ACTGTGGTGAGCTCATCTCTCAGGATGAGGCTCATCGACGCGGAAAGGTCTATGACAAATACATGTCCAG
CTTCCTCTTCAACCTCAATAATGATTTTGTAGTGGATGCTACTCGGAAAGGAAACAAAATTCGATTTGCA
ATCATTCAGTGAATCCCAACTGTTATGCACAAAGTGGTCATGGTGAATGGAGACCATCGGATTGGGATCT
TTGCCAAGAGGGCAATTCAAGCTGGCGAAGAGCTCTTCTTTGATTACAGGTACAGCCAAGCTGATGCTCT
CAAGTACGTGGGGATCGAGAGGGAGACCGACGTCCTTTAGCCCTCCCAGGCCCCAACGGCAGCACTTATG
GTAGCGGCACTGTCTTGGCTTTCGTGCTCACACCACTGCTGCTCGAGTCTCCTGCACTGTGTCTCCCACA
CTGAGAAACCCCCCAACCCACTCCCCCTGTAGTGAGGCCTCTGCCATGTCCAGAGGGCACAAAACTGTCT
CAATGAGAGGGGAGACAGAGGCAGCTAGGGCTTGGTCTCCCAGGACAGAGAGTTACAGAAATGGGAGACT
GTTT
EZH2
SEQ ID NO:78
>gi | 4758323 | ref | NM_004456.1 | Homo sapiens enhancer of zeste homolog 2
(Drosophila) (EZH2), mRNA
GAATTCCGGGCGACGCGCGGGAACAACGCGAGTCGGCGCGCGGGACGAAGAATAATCATGGGCCAAGCTG
GGAAGAAATCTGAGAAGGGACCAGTTTGTTGGCGGAAGCGTGTAAAATCAGAGTACATGCGACTGAGACA
GCTCAAGAGGTTCAGACGAGCTGATGAAGTAAAGAGTATGTTTAGTTCCAATCGTCAGAAAATTTTGGAA
AGAACGGAAATCTTAAACCAAGAATGGAAACAGCGAAGGATACAGCCTGTGCACATCCTGACTTCTGTGA
GCTCATTGCGCGGGACTAGGGAGTGTTCGGTGACCAGTGACTTGGATTTTCCAACACAAGTCATCCCATT
AAAGACTCTGAATGCAGTTGCTTCAGTACCCATAATGTATTCTTGGTCTCCCCTACAGCAGAATTTTATG
GTGGAAGATGAAACTGTTTTACATAACATTCCTTATATGGGAGATGAAGTTTTAGATCAGGATGGTACTT
TCATTGAAGAACTAATAAAAAATTATGATGGGAAAGTACACGGGGATAGAGAATGTGGGTTTATAAATGA
TGAAATTTTTGTGGAGTTGGTGAATGCCCTTGGTCAATATAATGATGATGACGATGATGATGATGGAGAC
GATCCTGAAGAAAGAGAAGAAAAGCAGAAAGATCTGGAGGATCACCGAGATGATAAAGAAAGCCGCCCAC
CTCGGAAATTTCCTTCTGATAAAATTTTGGAGGCCATTTCCTCAATGTTTCCAGATAAGGGCACAGCAGA
AGAACTAAAGGAAAAATATAAACAACTCACCGAACAGCAGCTCCCAGGCGCACTTCCTCCTGAATGTACC
CCCAACATAGATGGACCAATTGCTAAATCTGTTCAGAGAGAGCAAAGCTTACACTCCTTTCATACGCTTT
TCTGTAGGCGATGTTTTAAATATGACTGCTTCCTACATCCTTTTCATGCAACACCCAACACTTATAAGCG
GAAGAACACAGAAACAGCTCTAGACAACAAACCTTGTGGACCACAGTGTTACCAGCATTTGGAGGGAGCA
AAGGAGTTTGCTGCTGCTCTCACCGCTGAGCGGATAAAGACCCCACCAAAACGTCCAGGAGGCCGCAGAA
GAGGACGGCTTCCCAATAACAGTAGCAGGCCCAGCACCCCCACCATTAATGTGCTGGAATCAAAGGATAC
AGACAGTGATAGGGAAGCAGGGACTGAAACGGGGGGAGAGAACAATGATAAAGAAGAAGAAGAGAAGAAA
GATGAAACTTCGAGCTCCTCTGAAGCAAATTCTCGGTGTCAAACACCAATAAAGATGAAGCCAAATATTG
AACCTCCTGAGAATGTGGAGTGGAGTGGTGCTGAAGCCTCAATGTTTAGAGTCCTCATTGGCACTTACTA
TGACAATTTCTGTGCCATTGCTAGGTTAATTGGGACCAAAACATGTAGACAGGTGTATGAGTTTAGAGTC
AAAGAATCTAGCATCATAGCTCCAGCTCCCGCTGAGGATGTGGATACTCCTCCAAGGAAAAAGAAGAGGA
AACACCGGTTGTGGGCTGCACACTGCAGAAAGATACAGGTGAAAAAGGACGGCTCCTCTAACCATGTATA
CAACTATCAACCCTGTGATCATCCACGGCAGCCTTGTGACAGTTCGTGCCCTTGTGTGATAGCACAAAAT
TTTTGTGAAAAGTTTTGTCAATGTAGTTCAGAGTGTCAAAACCGCTTTCCGGGATGCCGCTGCAAAGCAC
AGTGCAACACCAAGCAGTGCCCGTGCTACCTGGCTGTCCGAGAGTGTGACCCTGACCTCTGTCTTACTTG
TGGAGCCGCTGACCATTGGGACAGTAAAAATGTGTCCTGCAAGAACTGCAGTATTCAGCGGGGCTCCAAA
AAGCATCTATTGCTGGCACCATCTGACGTGGCAGGCTGGGGGATTTTTATCAAAGATCCTGTGCAGAAAA
ATGAATTCATCTCAGAATACTGTGGAGAGATTATTTCTCAAGATGAAGCTGACAGAAGAGGGAAAGTGTA
TGATAAATACATGTGCAGCTTTCTGTTCAACTTGAACAATGATTTTGTGGTGGATGCAACCCGCAAGGGT
AACAAAATTCGTTTTGCAAATCATTCGGTAAATCCAAACTGCTATGCAAAAGTTATGATGGTTAACGGTG
ATCACAGGATAGGTATTTTTGCCAAGAGAGCCATCCAGACTGGCGAAGAGCTGTTTGTTGATTACAGATA
CAGCCAGGCTGATGCCCTGAGTATGTCGGCATCGAAAGAGAAATGGAAATCCCTTGACATCTGACTACCT
CCTCCCCCTCCTCTGAAACAGCTGCCTTAGCTTCAGGAACCTCGAGTACTGTGGGCAATTTAGAAAAAGA
ACATGCAGTTTGAAATTCTGAATTTGCAAAGTACTGTAAGAATAATTTATAGTAATGAGTAAAAAAATCA
ACTTTTTATTGCCTTCTCACCAGCTGCAAAGTGTTTTGTACCAGTGAATTTTTGCAATAATGCAGTATGG
TACATTTTTCAACTTTGAATAAAGAATACTTGAACTTGAAAAAAAAAAAAAAAAAA
BMI1
SEQ ID NO:79
>gi | 4885094 | ref | NM_005180.1 | Homo sapiens B lymphoma Mo-MLV insertion region
(mouse) (BMI1), mRNA
GAGAGGCAGAGATCGGGGCGAGACAATGGGGATGTGGGCGCGGGAGCCCCGTTCCGGCTTAGCAGCACCT
CCCAGCCCCGCAGAATAAAACCGATCGCGCCCCCTCCGCGCGCGCCCTCCCCCGAGTGCGGAGCGGGAGG
AGGCGGCGGCGGCCGAGGAGGAGGAGGAGGAGGCCCCGGAGGAGGAGGCGTTGGAGGTCGAGGCGGAGGC
GGAGGAGGAGGAGGCCGAGGCGCCGGAGGAGGCCGAGGCGCCGGAGCAGGAGGAGGCCGGCCGGAGGCGG
CATGAGACGAGCGTGGCGGCCGCGGCTGCTCGGGGCCGCGCTGGTTGCCCATTGACAGCGGCGTCTGCAG
CTCGCTTCAAGATGGCCGCTTGGCTCGCATTCATTTTCTGCTGAACGACTTTTAACTTTCATTGTCTTTT
CCGCCCGCTTCGATCGCCTCGCGCCGGCTCCTCTTTCGGGGATTTTTTATCAAGCAGAAATGCATCGAAC
AACGAGAATCAAGATCACTGAGCTAAATCCCCACCTGATGTGTGTGCTTTGTGGAGGGTACTTCATTGAT
GCCACAACCATAATAGAATGTCTACATTCCTTCTGTAAAAGTTGTATTGTTCGTTACCTGGAGACCAGCA
AGTATTGTCCTATTTGTGATGTCCAAGTTCACPAGACCAGACCACTACTGAATATAAGGTCAGATAAAAC
TCTCCAAGATATTGTATACAAATTAGTTCCAGGGCTTTTCAAAAATGAAATGAAGAGAAGAAGGGATTTT
TATGCAACTCATCTTCTGCTGATGCTGCCAATGGCTCTAATGAAAGATAGAGGAGAGGTTGCAGATGAAG
ATAAGAGAATTATAACTGATGATGAGATAATAAGCTTATCCATTGAATTCTTTGACCAGAACAGATTGGA
TCGGAAGTAAACAAAGACAAAGAGAAATCTAAGGAGGAGGTGAATGATAAAAAGATACTTACGATGCCCA
GCAGCAATGAGTGTGATGCACTTAAGAAAGTTTCTCAGAAGTAAAATGGACATACCTAATACTTTCCAGA
TTGATGTCATGTATGAGGAGGAACCTTTAAAGGATTATTATACACTAATGGATATTGCCTACATTTATAC
CTGGAGAAGGAATGGTCCACTTCCATTGAAATACAGAGTTCGACCTACTTGTAAAAGAATGAAGATCAGT
CACCAGACAGATGGACTGACAAATGCTGGAGAACTGGAAAGTGACTCTGGGAGTGACAAGGCCAACAGCC
CAGCAGGAGGAGTTCCCTCCACCTCTTCTTGTTTGCCTAGCCCCAGTACTCCAGTGCAGTCTCCTCATCC
ACAGTTTCCTCACATTTCCAGTACTATGAATGGAACCAGCAACAGCCCCAGCGGTAACCACCAATCTTCT
TTTGCCAATAGACCTCGAAAATCATCAGTAAATGGGTCATCAGCAACTTCTTCTGGTTGATACCTGAGAC
TGTTAAGGAAAAAAATTTTAAACCCCTGATTTATATAGATATCTTCAGCCATTACGACTTTCTAGAGCTA
ATACATGTGACTATCGTCCAATTTGCTTTCTTTAGTAGTGACATTAAATTTGGCTATAAAAGATGGACTA
CATGTGATACTCCTGTCCGTCTTGGTTCAAAAGAAAGATTGTTGTTATAAAGAATTGGTTTCTTGGAAAG
CAGGCAAGACTTTTTCTCTGTGTTAGGAAAGATGGGAAATGGTTTCTGTAACCATTGTTTGGATTTGGAA
GTACTCTGCAGTGGACATAAGCATTGGGCCATAGTTTGTTAATCTCAACTAACGCCTACATTACATTCTC
CTTGATCGTTCTTGTTATTACGCTGTTTTGTGAACCTGTAGAAAAACAAGTGCTTTTTATCTTGAAATTC
AACCAACGGAAAGAATATGCATAGAATAATGCATTCTATGATGCCATGTCACTGTGAATAACGATTTCTT
GCAGCTATTTAGCCATTTTGATTGCTGTTTGATTTATACTTCTCTGTTGCTACGCAAAACCGATCAAAGA
AAAGTGAACTTCAGTTTTACAATCTGTATGCCTAAAAGCGGGTACTACCGTTTATTTTACTGACTTGTTT
AAATGATTCGCTTTTGTAAGAATCAGATGGCATTATGCTTGTTGTACAATGCCATATTGGTATATGACAT
AACAGGAAACAGTATTGTATGATATATCTATAAATGCTATAAAGAAATATTGTGTTTCATGCATTCAGAA
ATGATTGTTAAAATTCTCCCAACTGGTTCGACCTTTGCAGATACCCATAACCTATGTTGAGCCTTGCTTA
CCAGCAAAGAATATTTTTAATGTGGATATCTAATTCTAAAGTCTGTTCCATTAGAAGCAATTGGCACATC
TTTCTATACTTTATATACTTTTCTCCAGTAATACATGTTTACTTTAAAAATTGTTGCAGTGAAGAAAAAC
CTTTAACTGAGAAATATGGAAACCTCTTAATTTTCCATTGGCTATGATGGAATTAATATTGTATTTTTAA
AAATGCATATTGATCACTATAATTCTAAAACAATTTTTTAAATAAACCAGCAGGTTGCTAAAAGAAGGCA
TTTTATCTAAAGTTATTTTAATAGGTGGTATAGCAGTATTTTTAAATTTAAGAGTTGCTTTTACAGTTAA
CAATGGAATATGCCTTCTCTGCTATGTCTGAAAATAGAAGCTATTTATTATGAGCTTCTACAGGTATTTT
TAAATAGAGCAAGCATGTTGAATTTAAAATATGAATAACCCCACCCAACAATTTTCAGTTTATTTTTTGC
TTGGTCGAACTTGGTGTGTGTTCATCAGTTATTTGTGAGGGTGTTTATACTCATATGAATATTGTTTCAT
GTTTGTAGGGAAATTGTAGCTAAATCATTTCATTGTCCCCAGTCTGCAATGAAGCACAATTCTATTGCTT
TGTCTTGCTTATAGTCATTAATCATTACTTTTACATATATTGCTGTTACTTCTGCTTTCTTTTAAAAATA
TAGTAAAGGATGTTTTATGAAGTCACAAGATACATATATTTTTATTTTGACCTAAATTTGTACAGTCCCA
TTGTAAGTGTTGTTTCTAATTATAGATGTAAAATGAATTTCATTTGTAATTGGAAAAAAATCCAATAAAA
AGGATATTCATTTAGAAAATAGCTAAGATCTTTAATAAAAATTTGATATGAAA
ZNF144 (MEL18)
SEQ ID NO:80
>gi | 6005963 | ref | NM_007144.1 | Homo sapiens zinc finger protein 144
(Mel-18) (ZNF144), mRNA
GAGAGCCCGAACAGGAAGAGGGTACAGCTTTGTGCAGGTCACATGCCCACTGCAGCCCTCCAGCCTCTGG
TCCCCAGAGCGGACTTTGGAAGCTGAACTGCTTTTGTTGCTGGAACACTTATGTTATAATTTACCCTGGG
TGGACCAGGGTCGTACAAAAGGGCAACGCTCCCCAGTCCCCCCACTCCCGACCCCGGAATCATGCATCGG
ACTACACGGATCAAAATCACAGAGCTGAACCCCCACCTCATGTGTGCCCTCTGCGGGGGGTACTTCATCG
ACGCCACCACTATCGTGGAGTGCCTGCATTCCTTCTGCAAAACCTGCATCGTGCGCTACCTGGAGACCAA
CAAATACTGCCCCATGTGTGACGTGCAGGTCCATAAAACCCGGCCGCTGCTGAGCATCAGGTCTGACAAA
ACACTTCAAGACATTGTCTACAATTGGTCCCTGGGCTTTTTAAAAGATGAGATGAAACGGCGGCGGGATT
TCTATGCAGCGTACCCCCTGACGGAGGTCCCCAACGGCTCCAATGAGGACCGCGGCGAGGTCTTGGAGCA
GGAGAAGGGGGCTCTGAGTGATGATGAGATTGTCAGCCTCTCCATCGAATTCTACGAAGGTGCCAGGGAC
CGGGATGAGAAGAAGGGCCCCCTGGAGAATGGGGATGGGGACAAAGAGAAAACAGGGGTGCGCTTCCTGC
GATGCCCAGCAGCCATGACCGTCATGCATCTTGCCAAGTTTCTCCGCAACAAGATGGATGTGCCCAGCAA
GTACAAGGTGGAGGTTCTGTACGAGGACGAGCCACTGAAGGAATACTACACCCTCATGGACATCGCCTAC
ATCTACCCCTGGCGGCGGAACGGGCGTCTCCCCCTCAAGTACCGTGTCCAGCCAGCCTGCAAGCGGCTCA
CCCTAGCCACGGTGCCCACCCCCTCCGAGGGCACCAACACCAGCGGGGCGTCCGAGTGTGAGTCAGTCAG
CGACAAGGCTCCCAGCCCTGCCACCCTGCCAGCCACCTCCTCCTCCCTGCCCAGCCCAGCCACCCCATCC
CATGGCTCTCCCAGTTCCCATGGGCCTCCAGCCACCCACCCTACCTCCCCCACTCCCCCTTCGACAGCCA
GTGGGGCCACCACAGCTGCCAACGGGGGTAGCTTGAACTGCCTGCAGACACCATCCTCCACCAGCAGGGG
GCGCAAGATGACTGTCAACGGCGCTCCCGTGCCCCCCTTAACTTGAGGCCAGGGACCCTCTCCCTTCTTC
CAGCCAAGCCTCTCCACTCCTTCCACTTTTTCTGGGCCCTTTTTTCCACTTCTTCTACTTTCCCCAGCTC
TTCCCACCTTGGGGGTGGGGGGCGGGTTTTATAAATAAATATATATATATATGTACATAGGAAAAACCAA
ATATACATACTTATTTTCTATGGACCAACCAGATTAATTTAAATGCCACAGGAAACAAACTTTATGTGTG
TGTGTATGTGTGGAAAATGGTGTTCATTTTTTTTGGGGGGGGTCTTGTGTAATTTGCTGTTTTTGGGGGT
GCCTGGAGATGAACTGGATGGGCCACTGGAGTCTCAATAAAGCTCTGCACCATCCTCGCTGTTTCCCAAG
GCAGGTGGTGTGTTGGGGGCCCCTCAGACCACAAAGCTTTAGGCATGATTCCAACTGGCTGCATATAGGA
GTCAGTTAGAATTGTTTCTTTCTCTCCCCGTTTCTCTCCCCATCTTGGCTGCTGTCCTGCCTCTGACCAG
TGGCCGCCCCCCGCGTTGTTGAATGTCCAGAAATTGCTAAGAACAGTGCCTTTTACAAATGCAGTTTATC
CCTGGTTCTGAGGAGCAAGTGCAGGGTGGAGGTGGCACCTGCATCACCTCCTCCTCTTGCAGTGGAAACT
TTGTGCAAAGAATAGATAGTTCTGCCTCTTTTTTTTTTTTTTCCTGTGTGTGTGGCCTTTGCATCATTTA
TCTTGTGGAAAAGAAGATTCAGGCCCTGAGAGGTCTCAGCTCTTGGAGGAGGGCTAAGGCTTTAGCATTG
TGAAGCGCTGCACCCCCACCAACCTTACCCTCACCGGGGAACCCTCACTAGCAGGACTGGTGGTGGAGTC
TCACCTGGGGCCTAGAGTGGAAGTGGGGGTGGGTTAACCTCACACAAGCACAGATCCCAGACTTTGCCAG
AGGCAAACAGGGAATTCCGCCGATACTGACGGGCTCCAGGAGTCGTCGCCACACTCG
SCML1
SEQ ID NO:81
>gi | 5803158 | ref | NM_006746.1 | Homo sapiens sex comb on midleg-like 1
(Drosophila) (SCML1), mRNA
ATCACTGGTCTCGCGTGCGCGTCACCAGGCCCGGTTTCCGGTGCCAGGACCTTTCCGAAGCGTCGAGTGG
CCTAACGGTCACAGCTGTCGCCCATCGGAGAGGCAGGACTACTGCGAGCAGTTTTACCGCGACCTCCGGA
GGCCGGCGTGACAGGCTCTGTCACTAAAATAGGAACCGAATATTGTATCTGACGCATCCTGTAATACTGA
AGAGCAACTGAAGACAGTTGATGATGTCCTTATTCATTGCCAGGTTATATATGATGCTCTGCAAAACCTG
GATAAGAAGATTGATGTGATTCGTAGAAAGGTTTCAAAAATCCAACGTTTCCATGCGAGATCCCTGTGGA
CAAATCATAAGCGATATGGATATAAAAAGCATTCTTACCGGCTTGTTAAAAAGCTTAAACTCCAGAAAAT
GAAGAAAAATGAGGTTTACGAGACATTCTCCTACCCTGAAAGTTACAGCCCCACTTTACCAGTGTCAAGG
CGTGAGAATAATTCCCCGAGCAACCTTCCAAGGCCATCCTTTTGCATGGAAGAATACCAGCGAGCTGAGC
TGGAGGAGGACCCGATCCTCAGCCGCACTCCGAGTCCAGTGCATCCCTCAGATTTCTCTGAGCATAATTG
TCAGCCGTATTATGCATCTGATGGTGCAACGTATGGAACTTCTTCAGGGCTCTGCCTTGGCAACCCTCGG
GCTGACAGCATCCACAACACTTACTCAACTGACCATGCTTCTGCAGCACCACCTTCAGTTACAAGGTCAC
CAGTTGAAAATGACGGTTACATAGAGGAAGGAAGCATCACTAAGCACCCTTCAACCTGGTCGGTGGAAGC
AGTGGTCCTATTTCTATAACATACAGATCCTCTTGCATTATGCCCTCTTGTCGACCTCTTCAGAAGCCAT
GAAATTGACGGGAAGGCTCTGCTCCTACTCACGAGTGACGTGAAGCTGAAGCACTTGGGGGTGAAGCTGG
GAACGGCTGTGAAGCTATGCTACTACATTGACCGACTAAAACAAGGAAAATGCTTTGAAAATTGAAAAAA
TCCTPGTGCAAATTTAGATTGGGCCAACTTCTAGAGGCACCAATGCCTTCTTAGTGTGGAATCATTTTTC
TGCCCTTTAGTCGTTTTTGTTTTGTAGAAAGTATCTCTCAAAATATATTATAGCTAGAATTGTAGAACTA
TGTTATAGTCCAGTCTACTTCTTTAAAAACCATTTAAACTGCTAGATAGTATTAGAATAGTCCAATAGAA
AATTCATTCTTTATAGGTCTTTAAAAATTACTTTTATTATATTGTTTACAAATATATTTCATGCAAGAAA
CAGAAAAAAAAAAAAACCCTTTGATTCTGGTTCATCTCGATACAGAGAACCAAAACAGCTAAGAGAGGTA
TTATCAGGGTTGACAACTCCTATGATTGAATCTATGGGAATTATTCCTCAGAAGAGAATTTAAAGGTGTA
CCCATATATATCTCTTTCTGGAGTATTTTATCTGTCTGATGTTGCAGTATTCTACAAGTTTCCAGAAAGA
GAATAGCCATATAAATTATTTTCCTTTCTGCTATTATTTCTCTATATGTTTTATTTATTCAGATTTAGAG
TAAAAAATAAGCATATAAACTTTTATTATGTGCTCTTAACAGTTTTAAGATAAACTATAGGATAGATAGA
ATGGTTATTTTATGCAAGAAATATTGTACCGCAAGGGTGGTTTGGATGAAGTCTGACTACTTTTTTTCAA
ACAAACTATTATATTAAAACTGTCATATTTTGGCTAAGTTTGGACCTATAACTACACTTTCATTGTTTGC
ATCTCTCTATGAAGATACGTCTGTCCAAACTTTTAAAAGGCATAACTGTATTTTATGTGTTTATTCTTTA
TATAGATAGTATTTTATATTTTATTCTCACCCGAAGTATTCACACAATCTTTTTAAAAAAAATTTGAAAT
GGCATTTTGTATTGCCACAGAGGTAGGATGAGCCATATATTAGTGAAATGTTTTATTTTGTAAAATATAA
ATGGATTATTTGCCATCATTAGTACCTCTCAACTTACTTTTTAGAGGACAAGAAACAATCTGTAGAAAGG
TTTCCATACAGGGAAGTTCTCCGTCCTATGCAATGTTTCTAATTAATTTGCTTAATTCTGAGCCATTAAT
CCTGCTACACTTTGAATGATACATTAATTCAGACTAATCTTTGGGGGCTTTATTTTGTAAGTTAGAACTT
TCAAGGGAAACATGTTCAACACTATTATTTTGTTATAAATTTATAACTTTGTTATTACATTGTGTAACAA
ATATAAGGTTTACGAGCTATGAGAATTGGTGCTATCACCATTAGCTATTTGCTGTAATGTCAAGAAAATG
TTCACCAGATGCAAGAATGTACCTTTTCTTTTTAGAAAGCCAAATGTACTTTAGACATGAATGCAACTAT
TTAAAGAATAGCTTCATCAATGTTATTCCTTACATGTCATAAGATTCTTACTTAAACTTGGTCTTCTTTC
AAATTGTTTGTATGAAGATGCTGTACCCACTTGAACAGTCCTCAGGTGTTTACATAAATACTATGTTTTA
CAGTTTTCATATTTTAAAATATTAATAAAGTTAAATCACAATAGT
SCML2
SEQ ID NO:82
>gi | 5174668 | ref | NM_006089.1 | Homo sapiens sex comb on midleg-like 2
(Drosophila) (SCML2), mRNA
GAAGCTCGGTGCCGGCTCGCGCGATCGGTGGGACAGAATTTCGTTGTTTTCACCGACGAGACTGGAGGAA
ACAACACCAAATAGGGATACCATGGGACAAACAGTGAATGAAGATTCCATGGATGTCAAGAAGGAGAATC
AAGAGAAAACTCCTCAGTCAAGTACATCTTCTGTACAAAGGGATGATTTCCACTGGGAGGAGTATTTGAA
AGAGACTGGGTCTATAAGTGCTCCTTCAGAGTGCTTCCGTCAGTCTCAGATTCCACCTGTGAATGATTTC
AAAGTTGGTATGAAATTGGAAGCCCGTGACCCTCGCAATGCCACTTCAGTATGTATTGCTACGGTTATTG
GAATTACTGGGGCCAGGTTACGGTTACGACTGGATGGTAGTGACAACAGAAATGATTTTTGGAGGCTTGT
CGATTCCCCAGACATACAACCTGTTGGGACATGTGAAAAGGAAGGAGACTTACTTCAACCTCCACTAGGG
TACCAGATGAATACATCCTCCTGGCCGATGTTCCTCTTAAAGACACTAAATGGGTCTGAAATGGCATCTG
CCACATTATTTAAGAAGGAACCACCAAAGCCCCCACTAAATAATAATTTTAAAGTGGGGATGAAACTGGA
TATTGACAAAAAGAACCCGTATCTCATCTGTCCTGCGACCATTGGAGATGTTAAAGGGGATGAAGTTCAT
ATCACATTTGATGGCTGGAGTGGAGCTTTTGATTACTGGTGCAAGTATGATTCTCGAGATATTTTCCCAG
CTGGGTGGTGTCGCCTGACAGGAGATGTATTACAACCCCCAGGAACTAGTGTTCCTATTGTAAAGAATAT
AGCAAAAACAGAGTCTTCTCCTTCCGAAGCAAGCCAGCATTCAATGCAGTCTCCACAGAAAACTACTCTA
ATATTACCAACACAGCAGGTCAGGAGATCAAGTCGAATTAAACCACCTGGACCTACTGCAGTCCCCAAAA
GGAGCAGTTCTGTTAAAAATATCACACCAAGGAAAAAAGGTCCAAACTCAGGAAAAAAAAAAAAAACTTT
GCCCGTGATATGTTCTACATCTGCAGCTTCTCTAAAATCGCTGACCAGAGACCGTGGCATGTTATATAAA
GATGTCGCTTCTGGGCCATGTAAAATAGTGATGTCTACAGTCTGTGTCTATGTAAACAAACATGGAAACT
TTGGCCCTCATCTGGATCCCAAGAGAATCCAGCAGCTGCCTGACCACTTCGGCCCGGGCCCGGTGAATGT
GGTGCTTCGCCGGATTGTGCAGGCCTGTGTGGATTGTGCCCTTGAAACTAAAACTGTTTTTGGATACCTG
AAGCCAGATAATCGTGGAGGAGAAGTGATAACTGCCTCCTTTGATGGGGAAACTCAATTCCATCCAGTCC
CTCCAGTGAACAGTGCATCATTTGCTCTTCGCTTTCTTGAGAACTTCTGCCACAGTCTGCAGTGTGATAA
CCTTTTGAGTAGCCAGCCTTTTAGTTCTTCCAGGGGTCATACTCACAGCTCTGCAGAGCATGATAAAAAT
CAGTCAGCAAAAGAAGATGTAACAGAAAGGCAAAGCACCAAACGATCTCCTCAGCAAACTGTACCATATG
TTGTTCCTCTCTCTCCTAAGCTCCCCAAAACAAAGGAGTATGCGTCTGAAGGAGAACCATTGTTTGCTGG
GGGAAGTGCCATTCCCAAAGAGGAGAATCTTTCAGAAGATTCTAAGAGCTCATCACTAAATTCAGGAAAT
TATTTGAATCCTGCCTGTAGAAATCCTATGTATAATTCATACTTCAGTCTCCCAGGATTTTCTCGAAGTG
TGCCAGGCACCACAAGTTCACCACTAGTTGGGGACATATCCCCCAAGAGCAGTCCCCATGAAGTTAAATT
CCAAATGCAGAGGAAAAGTGAAGCTCCAAGTTATATAGCTGTACCTGATCCCAGTGTCCTGAAACAAGGC
TTCTCTAAGGACCCTTCAACCTGGTCTGTGGATGAAGTGATACAGTTTATGAAACATACAGATCCTCAGA
TATCAGGCCCCCTCGCCGACCTCTTCAGGCAACATGAAATTGATGGGAAGGCTCTGTTCCTACTCAAGAG
TGATGTGATGATGAAGTATATGGGGCTGAAGCTGGGGCCAGCATTAAAGCTGTGTTACTACATTGAAAAG
CTTAAAGAAGGAAAATACAGTTAAAAAATGTGTAAGTTTAGATTGGACATAATTCTCAGGTGTACTGTTA
ACATTTTAATTTAAAAGTATTTCTCTTAGCAGTTTTTGTTTTGTAGACAGTTCCCATAAAAATATTTTAT
CAGAATTGCAGAACTGTAGTAACAGTTCAGTCAACTTTGTTTTTTTCCTGGAGTCACCAACCAGCTTTGG
GAGACACAGCCGCCACTCCCCCAGTCTACTTCTTTAAAAAGCATTTAACAGGTTAGTATTGGCATATTCA
AATTGGCAGTTCTTTATGTCTTTTTAAATTTTCATTGTACAGTTTACAAATATACTTAATGTAGTTAACA
GAGAAAAACCTTTGATTTTGGTTAACCTTTATATCTAGAACCAAAACAGCTAATCCCAAAAGGGGAAAAT
ATCAGGGATTGACAACTTCTATAATTAAATCCATGAGAATTTTTCCTCACTAGAGAATTTAAAGGTGCAC
CTGTAGATATCATCTTTTCTCAGATATTTTGTGTGATACTCTGTGGTGTTCTGTTCATGTTCTATCAGTA
TATCTAGAAAGGGAATAGCCATATAAATTATTTTCCTTTTATTATTCTCTGTATGTTGTATTTTGATCAT
ATTTAAAGGAAAAAAGCAAGCTTATAAGCTTTCATGAAGTGTTCTTACCAGTTTTTGATAAATTTTTTAA
ATTATAGGATAGAATTGTCATTTTATGCAGGAGATATTTATACTACGAGGGTTGTTTGGATGGAGTCAGA
TTAAATTTTTTCAGTGAAATTCCTATTATTTTAAAACTTTCCATATTTTCACTACGCTTGGACATTTAAC
TAGGCATTCTTTTCTTACATCTCTATATGAAGATACCTGTGTCCAAAATTTTTGAAGATATATATTGTAT
GTGTTTATTCTTCATATGGTACTTTACCATATTTATATATTGTTTTATACCTGTAGGTTTACACACAAGT
AAATCTTTTTTTCTTGAATTTAATCTGGGCACTTTGCACTGCCACAGGGGTGACGATGGAACTATGTATA
TAGTTAGGATGTTTTGATTTCGGTAAAAAATATATGTCCCATCGTTGCTATCACCAGTACCTCTCAGCTT
ACTCTTCAGGGGATATGAAACAATCTGTAGATTGGGTTTCCATAACAGGGAAGTCCGTCCTAATGCCAAG
TTTCTAATTAATTTGCTTAGTTCTGAGCCATTTATTCTGCTACACTTTGAAAGATATATTAGTTCTGACT
TATTGTTGGGGCTTTATTTTATTTTTATTTTTTTGAGATGGAGTTTCACTCTTGTGCCCAGGCTGGAGTG
CAATAGCTCGATCTCGGCTCACTGCAACCTCGCCTCCCTGGTTCAAGCAATTCTCCTGCCTCAGCGATTA
CAGGAATGCACCACCACGCCCAGCTAATTTTGTAGAGATGGGGTTTCTCCATGTTGGTCAGGCTGGTCTC
AAACTCCTGACCTCAGGTGATCCGCCCACCTCGGCCTTGCAAAATGCTGGGATTACAGGCATGAGCCACC
TGCCCAGCCATGTTTGGGGCTTTATTTTATAAGTTAGAACTTTGAAGAGGAAATGGTGCTATATGTTTAT
TGTTATTACTTTGTGTAACTTTGATGTAATGTTTATAAGCTATGAGAATCAGTTATAAAAGTATTAGCCA
TTTGTTGTAAATGCCAATAAAATATTCACCAGGGGCAAGAATGTACATTTTCTTTTTAGAAAACCAAATG
TACTTTAGACATGAATGCAACTATTTAAAGAATAGCTTCATTTATGTTATTCCTTACATGTCATAAGATT
CTTACTTAAACTTGGTCTTCTTTCAAGTTGTTTGTATGAAGATGCTGTACCCACTTGAACAGTCCTCAGG
TGTTTACATAAATACTATGTTTTACAGTTTTCATATTTTAAAATATTAATAAAGTTAATCGCAACGATTC
RING1
SEQ ID NO:83
>gi | 11863157 | ref | NM_002931.2 | Homo sapiens ring finger protein 1
(RING1), mRNA
GGCTGCTGTTTCTAAAACCCCTTTCCCTCTAACCCACACCACCTTTCTACTCACTGATGCCTTCAGGAAG
CCATAATGGATGGCACAGAGATTGCTGTTTCCCCTCGGTCACTGCATTCAGAACTCATGTGCCCTATCTG
CCTGGACATGCTGAAGAATACGATGACCACCAAGGAGTGCCTCCACAGATTCTGCTCTGACTGCATTGTC
ACAGCCCTACGGAGCGGGAACAAGGAGTGTCCTACCTGCCGAAAGAAGCTGGTGTCCAAGCGATCCCTAC
GGCCAGACCCCAACTTTGATGCCCTGATCTCTAAGATCTATCCTAGCCGGGAGGAATACGAGGCCCATCA
AGACCGAGTGCTTATCCGCCTGAGCCGCCTGCACAACCAGCAGGCATTGAGCTCCAGCATTGAGGAGGGG
CTACGCATGCAGGCCATGCACAGGGCCCAGCGTGTGAGGCGGCCGATACCAGGGTCAGATCAGACCACAA
CGATGAGTGGGGGGGAAGGAGAGCCCGGGGAGGGAGAAGGGGATGGAGAAGATGTGAGCTCAGACTCCGC
CCCTGACTCTGCCCCAGGCCCTGCTCCCAAGCGACCCCGTGGAGGGGGCGCAGGGGGGAGCAGTGTAGGG
ACAGGGGGAGGCGGCACTGGTGGGGTGGGTGGGGGTGCCGGTTCGGAAGACTCTGGTGACCGGGGAGGGA
CTCTGGGAGGGGGAACGCTGGGCCCCCCAAGCCCTCCTGGGGCCCCCAGCCCCCCAGAGCCAGGTGGAGA
AATTGAGCTCGTGTTCCGGCCCCACCCCTGCTCGTGGAGAAGGGAGAATACCTGCCAGACGAGGTATGTG
AAGACAACTGGGAATGCCACAGTGGACCACCTCTCCAAGTACTTGGCCCTGCGCATTGCCCTCGAGCGGA
GGCAACAGCAGGAAGCAGGGGAGCCAGGAGGGCCTGGAGGGGGCGCCTCTGACACCGGAGGACCTGATGG
GTGTGGCGGGGAGGGTGGGGGTGCCGGAGGAGGTGACGGTCCTGAGGAGCCTGCCTTTGCCAGCCTGGAG
GGCGTCAGTGAAAAGCAGTACACCATCTACATCGCACCTGGAGGCGGGGCGTTCACGACGTTGAATGGCT
CGCTGACCCTGGAGCTGGTGAATGAGAAATTCTGGAAGGTGTCCCGGCCACTGGAGCTGTGCTATGCTCC
CACCAAGGATCCAAAGTGACCCCACCAGGGGACAGCCAGAGGAAGGGGACCATGGGGTATCCCTGTGTCC
TGGTCTATCACCCCAGCTTCTTTGTCCCCCAGTACCCCCAGCCCAGCCAGCCAATAAGAGGACACAAATG
AGGACACGTGGCTTTTATACAAAGTATCTATATGAGATTCTTCTATATTGTACAGAGTGGGGCAAAACAC
GCCCCCATCTGCTGCCTTTTCTATTGCCCTGCAACGTCCCATCTATACGAGGTGTTGGAGAAGGTGAAGA
ACCCTCCCATTCACGCCCGCCTACCAACAACAAACGTGCTTTTTTCCTCTTTGAAAAAA
RYBP
SEQ ID NO:84
>GI | 6912639 | ref | NM_012234.1 | Homo sapiens RING1 and YY1 binding
protein (RYBP), mRNA
CCCGGACGGCGTTTCTCCTCCGAGCGGCGCCGGTTTCGGCTTGGGGGGGGCGGGGTACAGCCCATCCATG
ACCATGGGCGACAAGAAGAGCCCGACCAGGCCAAAAAGACAAGCGAAACCTGCCGCAGACGAAGGGTTTT
GGGATTGTAGCGTCTGCACCTTCAGAAACAGTGCTGAAGCCTTTAAATGCAGCATCTGCGATGTGAGGAA
AGGCACCTCCACCAGAAAACCTCGGATCAATTCTCAGCTGGTGGCACAACAAGTGGCACAACAGTATGCC
ACCCCACCACCCCCTAAAAAGGAGAAGAAGGAGAAAGTTGAAAAGCAGGACAAAGAGAAACCTGAGAAAG
ACAAGGAAATTAGTCCTAGTGTTACCAAGAAAAATACCAACAAGAAAACCAAACCAAAGTCTGACATTCT
GAAAGATCCTCCTAGTGAAGCAAACAGCATACAGTCTGCAAATGCTACAACAAAGACCAGCGAAACAAAT
CACACCTCAAGGCCCCGGCTGAAAAACGTGGACAGGAGCACTGCACAGCAGTTGGCAGTAACTGTGGGCA
ACGTCACCGTCATTATCACAGACTTTAAGGAAAAGACTCGCTCCTCATCGACATCCTCATCCACAGTGAC
CTCCAGTGCAGGGTCAGAACAGCAGAACCAGAGCAGCTCGGGGTCAGAGAGCACAGACAAGGGCTCCTCC
CGTTCCTCCACGCCAAAGGGCGACATGTCAGCAGTCAATGATGAATCTTTCTGAAATTGCACATGGAATT
GTGAAAACTATGAATCAGGGTATGAAATTCAAAACTTCCACCTGCCCATGCTGCTTGCATCCCTGGAGAA
TCTTCTGTGGACATCGACCTCTTAGTGATGCTGCCAGGATAATTTCTGCTTGCCATGGGCATCTGGCCAC
CAGGAATTTCGCACCCTGACGATTACTCTTGACACTTTTTATGTATTCCATTGTTTTATATGATTTTCCT
AACAATCATTTATAATTGGATGTGCTCCTGAATCTACTTTTAATAAAAAAAAAAAA
MLL
SEQ ID NO: 85
>gi | 5174568 | ref | NM_005933.1 | Homo sapiens myeloid/lymphoid or mixed-
lineage leukemia (trithorax homolog, Drosophila) (MLL), mRNA
ATGGCGCACAGCTGTCGGTGGCGCTTCCCCGCCCGACCCGGGACCACCGGGGGCGGCGGCGGCGGGGGGC
GCCGGGGCCTAGGGGGCGNCCCGCGGCAACGCGTCCCGGCCCTGCTGCTTCCCCCCGGGCCCCCGGTCGG
CGGTGGCGGCCCCGGGGCGCCCCCCTCCCCCCCGGCTGTGGCGACCGCGGCGGCGGCAGCGAGAAGAAGC
GGGGCTGGGGTTCCAGGGGGAGCGGCCGCCGCCTCAGCAGCCTCCTCGTCGTCCGCCTCGTCTTCGTCTT
CGTCATCGTCCTCAGCCTCTTCAGGGCCGGCCCTGCTCCGGGTGGGCCCGGGCTTCGACGCGGCGCTGCA
GGTCTCGGCCGCCATCGGCACCAACCTGCGCCGGTTCCGGGCCGTGTTTGGGGAGAGCGGCGGGGGAGGC
GGCAGCGGAGAGGATGAGCAATTCTTAGGTTTTGGCTCAGATGAAGAAGTCAGAGTGCGAAGTCCCACAA
GGTCTCCTTCAGTTAAAACTAGTCCTCGAAAACCTCGTGGGAGACCTAGAAGTGGCTCTGACCGAAATTC
AGCTATCCTCTCAGATCCATCTGTGTTTTCCCCTCTAAATAAATCAGAGACCAAATCTGGAGATAAGATC
AAGAAGAAAGATTCTAAAAGTATAGAAAAGAAGAGAGGAAGACCTCCCACCTTCCCTGGAGTAAAAATCA
AAATAACACATGGAAAGGACATTTCAGAGTTACCAAAGGGAAACAAAGAAGATAGCCTGAAAAAAATTAA
AAGGACACCTTCTGCTACGTTTCAGCAAGCCACAAAGATTAAAAAATTAAGAGCAGGTAAACTCTCTCCT
CTCAAGTCTAAGTTTAAGACAGGGAAGCTTCAAATAGGAAGGAAGGGGGTACAAATTGTACGACGGAGAG
GAAGGCCTCCATCAACAGAAAGGATAAAGACCCCTTCGGTCTCCTCATTAATTCTGAACTGGAAAAGCCC
CAGAAAGTCCGGAAAGACAAGGAAGGAACACCTCCACTTACAAAAGAAGATAAGACAGTTGTCAGACAAA
GCCCTCGAAGGATTAAGCCAGTTAGGATTATTCCTTCTTCAAAAAGGACAGATGCAACCATTGCTAAGCA
ACTCTTACAGAGGGCAAAAAAAGGGGGCTCAAAAGAAAATTGAAAAAGAAGCAGCTCAGCTGCAGGGAAG
AAAGGTGAAGACACAGGTCAAAAATATTCGACAGTTCATCATGCCTGTTGTCAGTGCTATCTCCTCGCGG
ATCATTAAGACCCCTCGGCGGTTTATAGAGGATGAGGATTATGACCCTCCAATTAAAATTGCCCGATTAG
AGTCTACACCGAATAGTAGATTCAGTGCCCCGTCCTGTGGATCTTCTGAAAAATCAAGTGCAGCTTCTCA
GCACTCCTCTCAAATGTCTTCAGACTGCCTCTCGATCTAGTAGCCCAGTGTTGATACCTCCACAGACTCT
CAGGCTTCTGAGGAGATTCAGGTACTTCCTGAGGAGCGGAGCGATACCCCTGAAGTTCATCCTCCACTGC
CCATTTCCCAGTCCCCAGAAAATGAGAGTAATGATAGGAGAAGCAGAAAGGTATCAGTGTCGGAGAGAAG
TTTTGGATCTAGAACGACGAAAAAATTATCAACTCTACAAAGTGCCCCCCAGCAGAAGACCTCCTCGTCT
CCACCTCCACCTCTGCTGACTCCACCGCCACCACTGCAGCGGCCTCAGTATCTCTGAAACCACACACCTT
GGCTTAATGCCTCCAACAATCCCCTAGCATCACCATTTTTGCCTGCTTCCACTGCTCCTATGCAAGGGAA
GCGAAAATCTATTTTGCGAGAACCGACATTTAGGTGGACTTCTTTAAAGCATTCTAGGTCAGAGCCACAA
TACTTTTCCTCAGCAAAGTATGCCAAAGAAGGTCTTATTCGCAAACCAATATTTGATAATTTCCGACCCC
CTCCACTAACTCCCGAGGACGTTGGCTTTGCATCTGGTTTTTCTGCATCTGGTACCGCTGCTTCAGCCCG
ATTGTTTTCGCCACTCCATTCTGGAACAAGGTTTGATATGCACAAAAGGAGCCCTCTTCTGAGAGCTCCA
AGATTTACTCCAAGTGAGGCTCACTCTAGAATATTTGAGTCTGTAACCTTGCCTAGTAATCGAACTTCTG
CTGGAACATCTTCTTCAGGAGTATCCAATAGAAAAAGGAAAAGAAAAGTGTTTAGTCCTATTCGATCTGA
ACCAAGATCTCCTTCTCACTCCATGAGGACAAGAAGTGGAAGGCTTAGTAGTTCTGAGCTCTCACCTCTC
ACCCCCCCGTCTTCTGTCTCTTCCTCGTTAAGCATTTCTGTTAGTCCTCTTGCCACTAGTGCCTTAAACC
CAACTTTTACTTTTCCTTCTCATTCCGTGACTCAGTCTGGGGAATCTGCAGAGALAAATCAGAGACCAAG
GAAGCAGACTAGTGCTCCGGCAGAGCCATTTTCATCAAGTAGTCCTACTCCTCCCTTCCCTTGGTTTACC
CCAGGCTCTCAGACTGAAAGAGGGAGAAATAAAGACAAGGCCCCCGAGGAGCTGTCGAAAGATCGAGATG
CTGACAAGAGCGTGGAGAAGGACAAGAGTAGAGAGAGAGACCGGGAGAGAGAAAAGGAGAATAAGCGGGA
GTCAAGGAAAGAGAAAAGGAAAAAGGGATCAGAAATTCAGAGTAGTTCTGCTTTGTATCCTGTGGGTAGG
GTTTCCAAAGAGAAGGTTGTTGGTGAAGATGTTGCCACTTCATCTTCTGCCAAAAAAGCAACAGGGCGGA
AGAAGTCTTCATCACATGATTCTGGGACTGATATTTCTTCTGTGACTCTTGGGGATACAACAGCTGTCAA
AACCAAAATACTTATAAAGAAAGGGAGAGGAAATCTGGAAAAAACCAACTTGGACCTCGGCCCAACTGCC
CCATCCCTGGAGAAGGAGAAAACCCTCTGCCTTTCCACTCCTTCATCTAGCACTGTTAAACATTCCACTT
CCTCCATAGGCTCCATGTTGGCTCAGGCAGACAAGCTTCCAATGACTGACAAGAGGGTTGCCAGCCTCCT
AAAAAAGGCCAAAGCTCAGCTCTGCAAGATTGAGAAGAGTAAGAGTCTTAAACAAACCGACCAGCCCAAA
GCACAGGGTCAAGAAAGTGAACTCATCAGAGACCTCTGTGCGAGGACCCGGATTAAACATGTCTGCAGAA
GAGCAGCTGTTGCCCTTGGCCGAAAACGAGCTGTGTTTCCTGATGACATGCCCACCCTGAGTGCCTTACC
ATGGGAGAACGAGAAAGATTTTGTCTCCGGGGAATGATGACAAGTCATCATCGAAATTGCTGGCTCAGAA
GATGCTGAACCTCTTGCTCCACCCATCAAACCAATTAAACCTGTCACTAGAAACAAGGCACCCCAGGAAC
CTCCAGTAAAGAAAGGACGTCGATCGAGGCGGTGTGGGCAGTGTCCCGGCTGCCAGGTGCCTGAGGACTG
TGGTGTTTGTACTAATTGCTTAGATAAGCCCCAGTTTGGTGGTCGCAATATAAAGAAGCAGTGCTGCAAG
ATGAGAAAATGTCAGAATCTACACATGGATGCCTCCGCCTACCTGCAGAAAGCAAGCTAATTGCTGATGA
AAAAGAAAGAGAAAAGTCTAAGACCAGTGAAAAGAAAGACAGCAAAGAGAGCAGTGTTGTGAAAGAACGT
GGTGGACTCTAGTCAGAAACCTACCCCATCAGCAAGACGGATCCTGCCCCAAAAGAAAAGCAGTAGTGAG
CCTCCTCCACGAAAGCCCGTCGAGGAAAAGAGTGAAGAAGGGAATGTCTCGGCCCCTGGGCCTGAATCCA
AACAGGCCACCACTCCAGCTTCCAGGAAGTCAAGCAAGCAGGTCTCCCAGCCAGCACTGGTCATCCCGCC
TCAGCCACCTACTACAGGACCGCCAAGAAGAAGTTCCCAACCACTCCTAGTAGAGTCCCAAAGGAAAAAG
CAGCCTCCACCACCAGAATCAGGTCCAGAGCAGAGCACAGAAAGTGGCTTCCCCGCCACAAAAAGTATCC
CTGTAAAACAAAAACCAAAAGAAAAGGAAAAACCACCTCCGGTCAATAAGCAGGAGAATGCAGGCACTTT
GAACATCCTCAGCACTCTCTCCAATGGCAATAGTTCTAAGCAAAAATATCCAGCAGATGGAGTCCACAGG
ATCAGAGTGGACTTTAAGGAGGATTGTGAAGCAGAAAATGTGTGGGAGATGGGAACTTAGGAAATCTTGA
CTTCTGTTCCTATAACACCCAGGGTGGTTTGCTTTCTCTGTGCCAGTAGTGGGCATGTAGAGTTTGTGTA
TTGCCAAGTCTGTTGTGAGCCCTTCCACAAGTTTTGTTTAGAGGAGAACGAGCGCCCTCTGGAGGACCAG
CTGGAAAATTGGTGTTGTCGTCGTTGCAAATTCTGTCACGTTTGTGGAAGGCAACATCAGGCTACAAAGC
AGCTGCTGGAGTGTAATAAGTGCCGAAACAGCTATCACCCTGAGTGCCTGGGACCAAACTACCCCACCAA
ACCCACAAAGAAGAAGAAAGTCTGGATCTGTACCAAGTGTGTTCGCTGTAAGAGCTGTGGATCCACAACT
CCAGGCAAAGGGTGGGATGCACAGTGGTCTCATGATTTCTCACTGTGTCATGATTGCGCCAAGCTCTTTG
CTAAAGGAAACTTCTGCCCTCTCTGTGACAAATGTTATGATGATGATGACTATGAGAGTAAGATGATGCA
ATGTGGAAAGTGTGATCGCTGGGTCCATTCCAAATGTGAGAATCTTTCAGATGAGATGTATGAGATTCTA
TCTAATCTGCCAGAAAGTGTGGCCTACACTTGTGTGAACTGTACTGAGCGGCACCCTGCAGAGTGGCGAC
TGGCCCTTGAAAAAGAGCTGCAGATTTCTCTGAAGCAAGTTCTGACAGCTTTGTTGAATTCTCGGACTAC
CAGCCATTTGCTACGCTACCGGCAGGCTGCCAAGCCTCCAGACTTAAATCCCGAGACAGAGGAGAGTATA
CCTTCCCGCAGCTCCCCCGAAGGACCTGATCCACCAGTTCTTACTGAGGTCAGCAAACAGGATGATCAGC
AGCCTTTAGATCTAGAAGGAGTCAAGAGGAAGATGGACCAAGGGAATTACACATCTGTGTTGGAGTTCAG
TGATGATATTGTGAAGATCATTCAAGCAGCCATTAATTCAGATGGAGGACAGCCAGAAATTAAAAAAGCC
AACAGCATGGTCAAGTCCTTCTTCATTCGGCAAATGGAACGTGTTTTTCCATGGTTCAGTGTCAAAAAGT
CCAGGTTTTGGGAGCCAAATAAAGTATCAAGCAACAGTGGGATGTTACCAAACGCAGTGCTTCCACCTTC
ACTTGACCATAATTATGCTCAGTGGCAGGAGCGAGAGGAAAACAGCCACACTGAGCAGCCTCCTTTAATG
AAGAAAATCATTCCAGTCCCAAACCCAAAGGTCCTGGAGAACCAGACTCACCAACATCCTCTGCATCCTC
CTACACCACCAATTTTGAGTACTGATAGGAGTCGAGAAGACAGTCCAGAGCTGAACCCACCCCCAGGCAT
AGAAGACAATAGACAGTGTGCGTTATGTTTGACTTATGGTGATGACAGTGCTAATGATGCTGGTCGTTTA
CTATATATTGGCCAAAATGAGTGGACACATGTAAATTGTGCTTTGTGGTCAGCGGAAGTGTTTGAAGATG
ATGACGGATCACTAAAGAATGTGCATATGGCTGTGATCAGGGGCAAGCAGCTGAGATGTGAATTCTGCCA
AAAGCCAGGAGCCACCGTGGGTTGCTGTCTCACATCCTGCACCAGCAACTATCACTTCATGTGTTCCCGA
GCCAAGAACTGTGCTTTCTGGATGATAAAAAAGTATATTGCCAACGACATCGGGATTTGATCAAAAGGCG
AAGTGGTTCCTGAGAATGGATTTGAAGTTTTCAGAAGAGTGTTTGTGGACTTTGAAGGAATCAGCTTGAG
AAGGAAGTTTCTCAATGGCTTGGAACCAGAAAATATCCACATGATGATTGGGTCTATGACAATCGACTGC
TTAGGAATTCTAAATGATCTCTCCGACTGTGAAGATAAGCTCTTTCCTATAGATATCAGTTTTTCCAGGG
TATACTGGAGCACCACAGATGCTCGCAAGCGCTGTGTATATACATGCAAGATAGTGGAGTGCCGTCCTCC
AGTCGTAGAGCCGGATATCAACAGCACTGTTGAACATGATGAAAACAGGACCATTGCCCATAGTCCAACA
TCTTTTACAGAAAGTTCATCAAAAGAGAGTCAAAACACAGCTGAAATTATAAGTCCTCCATCACCAGACC
GACCTCCTCATTCACAAACCTCTGGCTCCTGTTATTATCATGTCATCTCAAAGGTCCCCAGGATTCGAAC
ACCCAGTTATTCTCCAACACAGAGATCCCCTGGCTGTCGACCGTTGCCTTCTGCAGGAAGTCCTACCCCA
ACCACTCATGAAATAGTCACAGTAGGTGATCCATTACTCTCCTCTGGACTTCGAAGCATTGGCTCCAGGC
GTCACAGTACCTCTTCCTTATCACCCCAGCGGTCCACTCAAATAATAGTCTCTCCAATGAGAACTGGGAA
TACTTACTCTAGGAATAATGTTTCCTCAGTCTCCACCACCGGGACCGCTACTGATCTTGAATCAAGTGCC
AAAGTAGTTGATCATGTCTTAGGGCCACTGATTCAAGTACTAGTTTAGGGCAAAACTCATCCGACCTCTT
CAAATTTGCAAAGGACAGTGGTTACTGTAGGCAATAAAAACAGTCACTTGGATGGATCTTCATCTTCAGA
AATGAAGCAGTCCAGTGCTTCAGACTTGGTGTCCAAGAGCTCCTCTTTAAAGGGAGAGAAGACCAAAGTG
CTGAGTTCCAAGAGCTCAGAGGGATCTGCACATAATGTGGCTTACCCTGGAATTCCTAAACTGGCCCCAC
AGGTTCATAACACAACATCTAGAGAACTGAATGTTAGTAAAATCGGCTCCTTTGCTGAACCCTCTTCAGT
GTCGTTTTCTTCTAAAGAGGCCCTCTCCTTCCCACACCTCCATTTGAGAGGGCAAAGGAAAATCCGAGAC
CAACACACAGATTCTACCCAATCAGCAAACTCCTCTCCAGATGAAGATACTGAAGTCAAAACCTTGAAGC
TATCTGGAATGAGCAACAGATCATCCATTATCAACGAACATATGGGATCTAGTTCCAGAGATAGGAGACA
GAAAGGGAAAAAATCCTGTAAAGAAACTTTCAAAGAAAGCATTCCAGTAAATCTTTTTTTGGAACCTGGT
CAGGTGACAACTGGTGAGGAAGGAAACTTGAAGCCAGAGTTTATGGATGAGGTTTTGACTCCTGAGTATA
TGGGCCAACGACCATGTAACAATGTTTCTTCTGATAAGATTGGTGATAAAGGCCTTTCTATGCCAGGAGT
CCCCAAAGCTCCACCCATGCAAGTAGAAGGATCTGCCAAGGAATTACAGGCACCAGGAAACGCACAAGTC
AAAGTGACACTGACACCTCTAAAAATGGAAAATGAGAGTCAATCCAAAAATGCCCTGAAAGAAAGTAGTC
CTGCTTCCCCTTTGCAAATAGAGTCAACATCTCCCACAGAACCAATTTCAGCCTCTGAAAATCCAGGAGA
TGGTCCAGTGGCCCAACCAAGCCCCCAATAATACCTCATGCCAGGATTCTCAAGTAACAACTATCAGAAT
CTTCCAGTACAGGACAGAAACCTAATGCTTCCAGATGGCCCCAAACCTCAGGAGGATGGCTCTTTTAAAA
GAGGTATCCCCGTCGCAGTGCCCGTGCACGTTCTAACATGTTTTTTGGGCTTACCCCACTCTATGAAAGT
AAGATCCTATGGTGAAGAAGACATTCCATTCTACAGCAGCTCAACTGGGAAGAAGCGAGGCAAGAGATCA
GCTGAGGACAGGTGGATGGGGCCGATGACTTAAGCATTCAGATGAAGACGACTTATACTATTACAAAACT
TCACTAGAACAGTGATTTCTTCAGGTGGAGAGGAACGACTGGCATCCCATAATTTATTTCGGGAGGAGGA
ACAGTGTGATCTTCCAAAAATCTCACAGTTGGATGGTGTTGATGATGGGACACAGAGTGATACTAGTGTC
ACAGCCACAACAAGGAAAAGCAGCCAGATTCCAAAAAGAAATGGTAAAGAAAATGGAACAGAGAACTTAA
AGATTGATAGACCTGAAGATGCTGGGGAGAAAGAACATGTCACTAAGAGTTCTGTTGGCCACAAAAATGA
GCCAAAGATGGATAACTGCCATTCTGTAAGCAGAGTTAAAACACAGGGACAAGATTCCTTGGAAGCTCAG
CTCAGCTCATTGGAGTCAAGCCGCAGAGTCCACACAAGTACCCCCTCCGACAAAAATTTACTGGACACCT
ATAATACTGAGCTCCTGAAATCAGATTCAGACAATAACAACAGTGATGACTGTGGGAATATCCTGCCTTC
AGACATTATGGACTTTGTACTAGAATACTCCATCCATGCAGGCTTTGGGTGAGAGCCAAGAGTCGCATCT
TCATCAGAACTCCTGAATCTTGGTGAAGGATTGGGTCTTGACAGTAATCGTGAAAAAGACATGGGTCTTT
TTGAAGTATTTTCTCAGCAGCTGCCTACAACAGAACCTGTGGATAGTAGTGTCTCTTCCTCTATCTCAGC
AGAGGAACAGTTTGAGTTGCCTCTAGAGCTACCATCTGATCTGTCTGTCTTGACCACCCGGAGTCCAACT
GTCCCCAGCCAGAATCCCAGTAGACTAGCTGTTATCTCAGACTCAGGGGAGAAGAGAGTAACCATCACAG
AAAAATCTGTAGCCTCCTCTGAAAGTGACCCAGCACTGCTGAGCCCAGGAGTAGATCCAACTCCTGAAGG
CCACATGACTCCTGATCATTTTATCCAGGAACATGATGCAGACCACATCTCTAGCCCTCCATGTTGGAGT
TCAGTAGAGCAAGGTCATGGCAACAATCAGGATTTAACTAGGAACAGTAGCACCCCTGGCCTTCAGGTAC
CTGTTTCCCCAACTGTTCCCATCCAGAACCAGAAGTATGTGCCCAATTCTACTGATAGTCCTGGCCCGTC
TCAGATTTCCAATGCAGCTGTCCAGACCACTCCACCCCACCTGAAGCCAGCCACTGAGAAACTCATAGTT
GTTAACCAGAACATGCAGCCACTTTATGTTCTCCAAACTCTTCCAAATGGAGTGACCCAAAAAATCCAAT
TGACCTCTTCTGTTAGTTCTACACCCAGTGTGATGGAGACAAATACTTCAGTATTGGGACCCATGGGAGG
TGGTCTCACCCTTACCACAGGACTAAATCCAAGCTTGCCAACTTCTCAATCTTTGTTCCCTTCTGCTAGC
AAAGGATTGCTACCCATGTCTCATCACCAGCACTTACATTCCTTCCCTGCAGCTACTCAAAGTAGTTTCC
CACCAAACATCAGCAATCCTCCTTCAGGCCTGCTTATTGGGGTTCAGCCTCCTCCGGATCCCCAACTTTT
GGTTTCAGAATCCAGCCAGAGGACAGACCTCAGTACCACAGTAGCCACTCCATCCTCTGGACTCAAGAAA
AGACCCATATCTCGTCTACAGACCCGAAAGAATAAAAAACTTGCTCCCTCTAGTACCCCTTCAAACATTG
CCCCTTCTGATGTGGTTTCTAATATGACATTGATTAACTTCACACCCTCCCAGCTTCCTAATCATCCAAG
TCTGTTAGATTTGGGGTCACTTAATACTTCATCTCACCGAACTGTCCCCAACATCATAAAAAGATCTAAA
TCTAGCATCATGTATTTTGACCGGCACCCCTGTTACCACAGAGTGTGGGAGGAAACTGCTGCAACAGCGG
CAGGCACATCAACAATAAGCCAGGATACTAGCCACCTCACATCAGGGTCTGTGTCTGGCTTGGCATCCAG
TTCCTCTGTCTTGAATGTTGTATCCATGCAAACTACCACAACCCCTACAAGTAGTGCGTCAGTTCCAGGA
CACGTCACCTTAACCAACCCAAGGTTGCTTGGTACCCCAGATATTGGCTCAATAAGCAATCTTTTAATCA
AAGCTAGCCAGCAGAGCCTGGGGATTCAGGACCAGCCTGTGGCTTTACCGCCAAGTTCAGGAATGTTTCC
ACACTGGGGACATCTCAGACCCCCTCTACTGCTGCAAATAACAGCGGCATCTAGCATCTGTGTGCTCCCC
TCCACTCAGACTACGGGCATAACAGCCGCTTCACCTTCTGGGGAAGCAGACGAACACTATCAGCTTCAGC
ATGTGAACCAGCTCCTTGCCAGCAAAACTGGGATTCATTCTTCCCAGCGTGATCTTGATTCTGCTTCAGG
GCCCCAGGTATCCAACTTTACCCAGACGGTAGACGCTCCTAATAGCATGGGACTGGAGCAGAACAAGGCT
TTATCCTCAGCTGTGCAAGCCAGCCCCACCTCTCCTGGGGGTTCTCCATCCTCTCCATCTTCTGGACAGC
GGTCAGCAAGCCCTTCAGTGCCGGGTCCCACTAAACCCAAACCAAAAACCAAACGGTTTCAGCTGCCTCT
AGACAAAGGGAATGGCAAGAAGCACAAAGTTTCCCATTTGCGGACCAGTTCTTCTGAAGCACACATTCCA
GACCAAGAAACGACATCCCTGACCTCAGGCACAGGGACTCCAGGAGCAGAGGCTGAGCAGCAGGATACAG
CTTGAACCCTCACGGCTCAGCCAGGGCTGAAGTCCACCTCAGGAAGTCAGCATTTGACATGTTTAACTTC
CTGGTCTAACATCGTCAGCCTCCTGATACAACCCCAATGATGAAAGAAAGCAGGATGGAGGTACAGCTGA
AGTCAGCTCGGAGGGCAACTAGCATGGATCTGCCAATGCCCATGCGCTTCAGAACTTTAAAAAAGACTTC
TAAGGAGGCAGTTGGTGTCTACAGGTCTCCCATCCATGGCCGGGGTCTTTTCTGTAAGAGAACCATTGAT
GCAGGTGAGATGGTGATTGAGTATGCCGGCAACGTCATCCGCTCCATCCAGACTGACAAGCGGGAAAAGT
ATTACGACAGCAAGGGCATTGGTTGCTATATGTTCCGAATTGATGACTCAGAGGTAGTGGATGCCACCAT
GCATGGAATCGTGCACGCTTCATCAATCACTCGTGTGAGCCTAACTGCTATTCTCGGGTCATCAATATTT
GATGGGCAGAAGCACATTGTCATCTTTGCCATGCGTAAGATCTACCGAGGAGAGGAAACTCACTTAAACT
ATAAGTTCCCCATTGAGGATGCCAGCACAAGCTGCCCTGCAACTGTGGCGCCAAGAAATGCCGGATGGTT
CCTAAACTAA
MLLT1
SEQ ID NO:86
>gi | 5174570 | ref | NM_005934.1 | Homo sapiens
myeloid/lymphoid or mixed-lineage leukemia (trithorax
homolog, Drosophila); translocated to, 1 (MLLT1), mRNA
ATGGACAATCAGTGCACCGTCCAGGTGAGGTTAGAGCTGGGGCATCGCGCCCAACTGCGCAAGAAGCCCA
CCACGGAGGGGTTCACTCACGACTGGATGGTGTTTGTCCGCGGCCCCGAGCAATGTGACATCCAGCACTT
CGTGGAGAAGGTGGTCTTCTGGCTGCACGACAGCTTCCCCAAGCCCAGACGCGTGTGCAAGGAGCCCCCC
TACAAAGTAGAGGAGTCGGGGTACGCTGGCTTCATCATGCCCATCGAGGTGCACTTCAAAAACAAGGAGG
AGCCGAGGAAGGTCTGCTTCACCTACGACCTGTTCCTGAACCTTGGAAGGCAACCCGCCCGTGACCACCT
GCGCTGCGAGAAGCTCACCTTCAACAACCCCACCACGGAGTTCCGGTACAAGCTCCTGCGGGCCGGCGGG
GTGATGGTAATGCCCGAAGGAGCAGACACGGTGTCCAGGCCCAGTCCCGACTACCCCATGTTACCCACAA
TTCCACTCTCTGCCTTCTCTGACCCCAAGAGACCAATTCCATCCCACGGCTCCAAGGACGCCAACAAGGA
GAGCAGCAAGACCTCCAAGCCACACAAGGTGACCAAGGAGAACCGGGAGCGCCCCCGCAAAGACTCCGAG
AGCAAGAGCTCCTCCAAGGAGCTGGAGCGTGAGCAGGCCAAAAGCTCCAAGGACACCTCGCGGAAGCTGG
GCGAGGGCCGGCTGCCCATTGGAGGAGAGGCGCCACCGCCCAAGGCTGCCTTCAAGGAACCCAAGATGGC
CCTGAAAGAGACCAAGCTGGAAAGCACGTCCCCCAACCCTGGGCCCCCACCCCCACCCCCACCCCCACCC
CGGGCTTCCAGCAAGCGGCCGGCCACCGCCGACTCGCCAAAGCCCAGCGCCAAGAAGCAGAAGAAGAGCA
GCTCGAAGGGGTCCCGGAGTGCTCCAGGCACCTCGCCCCGCACCTCCTCCTCCTCCTCCTTCTCGGACAA
GAAGCCGGCCAAGGACAAGAGCAGCACCAGAGGGGAGAAGGTGAAGGCCGAGAGTGAGCCCCGGGAGGCC
AAAAAGGCCCTGGAGGTGGAGGAGTCCAACTCAGAGGACGAGGCCTCCTTCAAGTCCGAGTCTGCCCAGT
CAAGCCCGTCCAACTCCAGCTCCAGCTCAGACTCCAGCTCAGACTCAGACTTCGAGCCATCCCAGAACCA
CAGCCAAGGACCCCTGCGCTCCATGGTGGAGGACCTGCAGTCCGACGAGTCCGACGAGGACGACTCTTCG
TCAGGCGAGGAGGCTGCCGGCAAGACCAACCCGGGGAGGGACTCCAGGTTGAGCTTCAGCGACAGCGAGA
GTGACAACAGCGCCGACTCCTCCCTGCCCAGCCGTGAGCCCCCACCCCCCCAGAAGCCACCCCCGCCCAA
CAGCAAGGTGTCAGGCCGGAGGAGCCCCGAGTCCTGCAGCAAGCCTGAGAAGATCCTCAAGAAGGGCACC
TACGACAAGGCCTACACGGATGAGCTGGTGGAGCTACACCGGAGGCTGATGGCGCTGCGGGAGCGCAACG
TGCTGCAGCAGATTGTGATCTGATCGAGGAGACTGGCCACTTCAATGTCACCAACACCACCCTTCGACTT
CGACCTCTTCTCCCTGGACGAGACCACCGTGCGCAAACTGCAGAGCTGCCTGGAGGCCGTGGCCACATGA
MLLT7
SEQ ID NO:87
>gi | 5174578 | ref | NM_005938.1 | Homo sapiens myeloid/lymphoid or mixed-
lineage leukemia (trithorax homolog, Drosophila); translocated to, 7
(MLLT7), mRNA
GGGACAGCTTAGGGACTATCGTCCTGGGACTAGGGGGAAGTTCGCGACTTTCTGAAGACTGGCAGGAATG
TGCCTCCTGGCCCTCGATGCTTCCCCCCTGAGGGGAGGCATCGTGAGGGACTGTGGCAGGCTTCACTGAA
CGCTGAGCCGGGGAGGTCCAACTCCACGTATGGATCCGGGGAATGAGAATTCAGCCACAGAAGGCCGCCG
CGATCATAGACCTAGATCCCGACTTCGAACCCCAGAGCCGTCCCCGCTCCTGCACCTGGCCCCTTCCCCG
ACCAGAGATCGCTAACCAGCCGTCCGAGCCGCCCGAGGTGGAGCCAGATCTGGGGGAAAAGGTACACACG
GAGGGGCGCTCAGAGCCGATCCTGTTGCCCTCTCGGCTCTCAGAGCCGGCCGGGGGCCCCCAGCCCGGAA
TCCTGGGGGCTGTAACAGGTCCTCGGAAGGGAGGCTCCCGCCGGAATGCCTGGGGAAATCAGTCATATGC
AGAATTCATCAGCCAGGCCATTGAAAGCGCCCCGGAGAAGCGACTGACACTTGCCCAGATTTACGAGTGG
ATGGTCCGTACTGTACCCTACTTCAAGGACAAGGGTGACAGCAACAGCTCAGCAGGATGGAAGAACTCGA
TCCGCCACAACCTGTCCCTGCACAGCAAGTTCATCAAGGTTCACAACGAGGCCACCGGCAAAAGCTCTTG
GTGGATGCTGAACCCTGAGGGAGGCAAGAGCGGCAAAGCCCCCCGCCGCCGGGCCGCCTCCATGGATAGC
AGCAGCAAGCTGCTCCGGGGCCGCAGTAAAGCCCCCAAGAAGAAACCATCTGTGCTGCCAGCTCCACCCG
AAGGTGCCACTCCAACGAGCCCTGTCGGCCAGTTTGCCAAGTGGTCAGGCAGCCCTTGCTCTCGAAACCG
TGAAGAAGCCGATATGTGGACCACCTTCCGTCCACGAAGCAGTTCAAATGCCAGCAGTGTCAGCACCCGG
CTGTCCCCCTTGAGGCCAGAGTCTGAGGTGCTGGCGGAGGAAATAACAGCTTCAGTCAGCAGTTATGCAG
GGGGTGTCCCTCCCACCCTCAATGAAGGTCTAGAGCTGTTAGATGGGCTCAATCTCACCTCTTCCCATTC
CCTGCTATCTCGGAGTGGTCTCTCTGGCTTCTCTTTGCAGCATCCTGGGGTTACCGGCCCCTTACACACC
TACAGCAGCTCCCTTTTCAGCCCAGCAGAGGGGCCCCTGACAGCAGGAGAAGGGTGCTTCTCCAGCTCCC
AGGCTCTGGAGGCCCTGCTCACCTCTGATACGCCACCACCCCCTGCTGACGTCCTCATGACCCAGGTAGA
TCCCATTCTGTCCCAGGCTCCGACTCTTCTGTTGCTGGGGGGGCTTCCTTCCTCCAGTAAGCTGGCCACG
GGCGTCGGCCTGTGTCCGAAGCCCCTAGAGGCTCGAGGCCCCAGCAGTCTGGTTCCCACCCTTTCTATGA
TAGCACCACCTCCAGTCATGGCAAGTGCCCCCATCCCCAAGGCTCTGGGGACTCCTGTGCTCACACCCCC
TACTGAAGCTGCAAGCCAAGACAGAATGCCTCAGGATCTAGATCTTGATATGTATATGGAGAACCTGGAG
TGTGACATGGATAACATCATCAGTGACCTCATGGATGAGGGCGAGGGACTGGACTTCAAGTTTGAGCCAG
ATCCCTGAGTCATGCCTGGAAGCTTTGTCCCCTGCTTCAGATGTGGAGCCAGGCGTGTTCATATCTAGTC
TTTACCCTTGAGCCCTCCCCAGGAATTTGGGACCCTGCTTTAGAGCTAGGGTGGGGTCTGGTCACACACA
GGTGTTGAAGAAATTATAAAGATAAAGCTGCCCCATCTGGGGAGGATATGGGGAGGGAGATGGGAGGGGA
AAGGGGAGAGGGTTTTCTCACTGTGCCAATATAGGGGGTAAGGCCCCCTCTCAGGAGCCATCATCGGCTT
TCCCCATTCCTACCCACTTAGGCTTTGTAGCAAGATGAGCAATGCTGTTGGAAATGTGAAGTCACCAGTG
GCCTTACCCCTGCCTTTGGGAGCAGGATTTTTTTGTAGAGAGTCTTATCTGAGCTGAGCCAGGCTAGCTG
GAGCCTGGGATTTCTATGCAGTGGCCCCTTAGGCCAGTGATGTGCGGTGGGTGGGCTGTTTAGGGGATCT
GGAAGGGCCAGGTCTGAGCACTGGAGTGGCTCGCCAGGCCAAAATCACCCTTAGAAGGCTGCAGATAACA
GAAAGGCTTTTTATAAACTTTTAAAGAAATATAAACACAAATATAGAGATTTTTTAACCATGGCAGGGTG
CTAGTGGTGGGCAGAATGCTTTTTTTTCTTTCTGAAGGCTTTGTGATAGTGACATGATACAAACACTACA
GACAATAAATATTAGGAGACACAGGGAAGTGGGGAGAGGTGGGGAGTAATAGTAAACACAGGGAAGAGCT
CCCCTACGGACCAGGTATAGAGAAAGGTCTATGCAGAAATAGGTTAGAGTTTCCCTAACAAAAAAGCTAA
CCCAGGTCCCCTCATTCCTTCAACTTGTGCGTGGGAGTGTGTGGTGTTAGGGTGCAGCCACACTCTTCTA
TGACCCAGCATGGGTTAGTGCTATGGTGGGAGAGTACATTGAAGGCCTGGAATTAGCTTGGGGCCAGGGA
AGGGACTGGGAGGGGAGAGAAGAGAAGGAGGGAAGGATTTAGGATGGTAAAGTTAGGTACAGAGAGCTCC
CTGTTCAAGGCCCCTGACAGCTGTCCCTGCCCTTCTTCCCCTTCCTGACTGCAGGGGTTATGTGAGAAGT
GTGTGTGGCAGCAGGCAGCGGGGAGGGGAGGAACAGGGAAGGGGGAGCTGGGGAGCTTGGCTGAGGGTCT
GGGAAATGAGCAGGGATGGGGGGGGATGTGGATCAGGTTTACTAGCACCTGCCAGGGAGGCCATCTGGGG
CTCCTTCTCCACCCCAGCCCCCAAAGCAGCCCTTCCCCCAGTGCCCTTTGCATCGTCCCCTCCCCCACCC
AGTGGGGAAGTGTGTCAAATCAATAAATGAATAAATTCAATAAATGCCTATAACCAGCAGAAAAAAAAAA
AAAAAAAAAAAAAAAAAAAAAA
MLLT6
SEQ ID NO:88
>gi | 5174576 | ref | NM_005937.1 | Homo sapiens myeloid/lymphoid or mixed-
lineage luekemia (trithorax homolg, Drosophila); translocated to, 6
(MLLT6), mRNA
ATGAAGGAGATGGTAGGAGGCTGCTGCGTATGTTCGGACGAGAGGGGCTGGGCCGAGAACCCGCTGGTCT
ACTGCGATGGGCACGCGTGCAGCGTGGCCGTCCACCAAGCTTGCTATGGCATCGTTCAGGTGCCAACGGG
ACCCTGGTTCTGCCGGAAATGTGAATCTCAGGAGCGAGCAGCCAGGGTGAGGTGTGAGCTGTGCCCACAC
AAAGACGGGGCATTGAAGAGGACTGATAATGGAGGCTGGGCACACGTGGTGTGTGCCCTCTACATCCCCG
AGGTGCAATTTGCCAACGTGCTCACCATGGAGCCCATCGTGCTGCAGTACGTGCCTCATGATCGACCTGT
CAAGACGTGTTACATGTGCGAGGAGACGGGCCGGGAGAGCAAGGCGGCCTCGGGAGCCTGCATGACCTGT
AACCGCCATGGATGTCGACAAGCTTTCCACGTCACCTGTGCCCAAATGGCAGGCTTGCTGTGTGAGGAAG
AAGTGCTGGAGGTGGACAACGTCAAGTACTGCGGCTACTGCAAATACCACTTCAGCAAGATGAAGACATC
CCGGCACAGCAGCGGGGGAGGCGGAGGAGGCGCTGGAGGAGGAGGTGGCAGCATGGGGGGAGGTGGCAGT
GGTTTCATCTCTGGGAGGAGAAGCCGGTCAGCCTCAGCATCCACGCAGCAGGAGAAGCACCCCACCCACC
ACGAGAGGGGCCAGAAGAAGAGTCGAAAGGACAAAGAACGCCTTAAGCAGAAGCACAAGAAGCGGCCTGA
GTCGCCCCCCAGCATCCTCACCCCGCCCGTGGTCCCCACTGCTGACAAGGTCTCCTCCTCGGCTTCCTCT
TCCTCCCACCACGAGGCCAGCAGGCAGGAGACCTCTGAGAGCAGCAGGGAGTCAAAGGGGAAAAAGTCTT
CCAGCCATAGCCTGAGTCATAAAGGGAAGAAACTGAGCAGTGGGAAAGGTGTGAGCAGTTTTACGTCCGC
CTCCTCTTCTTCCTCCTCCTCTTCCTCCTCCTTTGGGGGGCCTTTCCAGCGTGCAGTCTCGTCCCTGCAG
AGCTCCCCTGACTTCTCTGCATTCCCCAAGCTGGAGCAGCCAGAGGAGGACAAGTACTCCAAGCCCACAG
CCCCCGCCCCTTCAGCCCCTCCTTCTCCCTCAGCTCCCGAGCCCCCCAAGGCTGACCTTTTTGAGCAGAA
GGTGGTGTTCTCTGGCTTTGGGCCCATCATGCGCTTCTCCACCACCACGTCCAGCTCAGGCCGGGCCGGG
GCGCCCTCCCCTGGGGACTATAAGTCTCCCCACGTCACGGGGTCTGGGGCCTCGGCAGGCACCCACAAAC
GGATGCCCGCACTGAGTGCCACCCCTGTGCCTGCTGATGAGACCCCTGAGACAGGCCTGAAGGAGAAGAA
GCACAAAGCCAGCAAGAGGAGCCGCCATGGGCCAGGCCGTCCCAAGGGCAGCCGGAACAAGGAGGGCACT
GGGGGCCCAGCTGCCCCATCCTTGCCCAGTGCCCAGCTGGCTGGCTTTACCGCCACTGCTGCCTCACGCT
TCTCTGGAGGTTCCCTGGTCAGCTCCGGCCTGGGAGGTGTGTCCTCCCGAACGTTTGGGCCTTCTGGGAG
CTTGCCCAGCTTGAGCCTGGAGTCCCCCTTACTAGGGGCAGGGATCTACACCAGTAATAAGGACCCGATC
TCCCACAATGGCGGGATGCTGCGGGCTGTCTGCAGCACCCCTCTCTCGTCCAGCCTCCTGGGGCGCCCAG
GGACCTCGGCCCTGCCCCGCCTCAGCCGCTCCCCGTTCACCAGCACCCTCCCCTCCTGTTCTGCTTCTAT
CTCCACCACTCAGGTGTTTTCTCTGGCTGGCTCTACCTTTAGCCTCCCTTCTACCCACATCTTGGAAACC
CCCATGGGTGCCGTTAATCCCCTCCTCTCCCAAGCTGAGAGCAGCCACACAGAGCCAGACCTGGAGGACT
GCAGCTTCCGGTGTCGGGGGACCTCCCCTCAGGAGAGTCTGTCTTCCATGTCCCCCATCAGCAGCCTCCC
CGCACTCTTCGACCAGACAGCCTCTGCACCCTGTGGGGGCGGCCAGTTAGACCCGGCGGCCCCAGGGACG
ACTAACATGGAGCAGCTTCTGGAGAAGCAGGGCGACGGGGAGGCCGGCGTCAACATCGTGGAGATGCTGA
AGGCGCTGCACGCGCTGCAGAAGGAGAACCAGCGGCTGCAGAGCAGATCCTGAGCCTGACGGCCAAAAAA
GGAGCGGCTGCAGATTCTCAAGGTGCAGCTCTGTGTGCCCTTCCCTGCCCTGCCTGCTGCCGTGCCTGCC
GCCAACGGCCCTGTCCCTGGGCCCTATGGCCTGCCTCCCCAAGCCGGGAGCAGCGACTCCTTGAGCACCA
GCAAGAGCCCTCCGGGGAAAGAGCACCTCGGCCTGGACAACTCGCTGTCCACTTCTTCTGAGGACCCACA
CTCAGGCTGCCCGAGCCGCAGCAGCTCGTCGCTGTCCTTCCACAGCACGCCCCCACCGCTGCCCCTCTTC
CAGCAGAGCCCTGCCACTCTGCCCCTTGGCCCTGCGTGGGGCCCCTGCCCCAGTCCCGCCCCAGCCGCGA
ACGGGTTAGGCCGGGCACCCGGGGCAGCGGGGCTGGGGGCCATGCCCATGGCTGAGGGGCTGTTGGGGGG
GCTGGCAGGCAGTGGGGGCCTGCCCCTCAATGGGCTCCTTGGGGGGTTGAATGGGGCCGCTGCCCCCAAC
CCCGCAAGCTTGAGCCAGGCTGGCGGGGCCCCCACGCTGCAGCTGCCAGGCTGTCTCAACAGCCTTACAG
AGCAGCAGAGACATCTCCTTCAGCAGCAAGAGCAGCAGCTCCAGCAAATCCAGCAGCTCCTGGCCTCCCC
GCAGCTGACCCCGGAACACCAGACTGTTGTCTACCAGATGATCCAGCAGATCCAGCAGAAACGGGAGAAG
CAGCGTCTGCAGATGGCTGGGGGCTCCCAGCTGCCCATGGCCAGCCTGCTGGCAGGAAGCTCCACCCCGC
TGCTGTCTGCGGGTACCCCTGGCCTGCTGCCCACAGCGTCTGCTCCACCCCTGCTGCCCGCTGGAGCCCT
AGTGGCTCCCTCGCTTGGCAACAACACAAGTCTCATGGCCGCAGCAGCTGCAGCTGCAGCAGTAGCAGCA
GCAGGCGGACCTCCAGTCCTCACTGCCCAGACCAACCCCTTCCTCAGCCTGTCGGGAGCAGAGGGCAGTG
GCGGTGGCCCCAAAGGAGGGACCGCTGACAAAGGAGCCTCAGCCAACCAGGAAAAAGGCTAA
MLLT4
SEQ ID NO:89
>gi | 5174574 | ref | NM_005936.1 | Homo sapiens myeloid/lymphoid or mixed-
lineage leukemia (trithorax homolog, Drosophila); translocated to, 4
(MLLT4), mRNA
ATGTCGGCGGGCGGCCGTGACGAGGAGCGGCGGAAGCTGGCCGACATCATCCACCACTGGAACGCCAACC
GGCTGGACCTGTTCGAGATCAGCCAGCCGACCGAGGATTTGGAGTTCCATGGAGTGATGAGATTTTATTT
TCAAGATAAAGCTGCTGGAAACTTAGCAACAAAATGTATTCGGGTC7CTAGTACTGCCACCACTCAAGAT
GTAATCGAAACGCTCGCGGAGAAATTTCGACCTGATATGCGAATGCTGTCCTCTCCCAAGTATTCACTCT
ATGAAGTGCATGTCAGCGGAGAAAGAAGATTGGATATAGATGAGAAACCTCTAGTTGTACAACTGAATTG
GAACAAGATGATCGGGAAGGCAGATTTGTTCTTAAGAATGACAAATGACGCCATTCCTCCTAAGGCTCAA
AGTAATGGACCTGAAAAAGCAGGAAAAGAAGGGGTTATCCAGAACTTCAAGAGAACTCTCTCAAAGAAAG
AAAAAGAAAGGAAAAGAAGAGAGAAAAAAGAGGCATTGCGACAGGCATCTGATAAAGATGATAGACCTTT
CCAAGGGGAGGATGTTGAAAATTCTCGACTGGCTGCTGAGGTTTACAATGACATGCCGGAAACCAGCTTT
ACTCGAACCATTTCTAATCCTGAGGTGGTTATGAAACGACGGAGGCAGCAAAAATTGGAAAAGAGAATGC
AGGAATTTCGGAGCTCAGATGGGCGGCCTGATTCAGGTGGAACATTGAGAATTTATGCAGATAGTTTAAA
ACCAAATATTCCCTACAAGACAATCCTGCTGTCTACTACAGATCCTGCAGACTTTGCTGTGGCTGAAGCT
TTAGAGAAGTATGGTCTGGAAAAAGAAAACCCTAAGGATTACTGCATCGCCCGGGTTATGCTTCCTCCTG
GAGCCCAGCATTCTGATGAAAAGGGTGCTAAAGAAATTATTCTTAATGATGATGAGTGTCCTTTACAAAT
CTTCAGGGAATGGCCAAGTGACAAAGGGATTTTAGTCTTTCAGTTGAAGAGGAGGCCACCAGACCACATC
CCAAAGAAAACCAAGAAACACTTGGAAGGCAAGACACCCAAGGGAAAGGAGAGAGCTGACGGGTCTGTCT
ATGGCTCCACCCTTCCTCCGGAGAAGCTGCCCTATTTAGTAGAGTTAAGCCCAGATGGTTCTGACTCTAG
AGATAAGCCAAAGCTTTACCGCCTTCAGTTAAGTGTTACTGAAGTTGGGACAGAAAAGTTGGATGACAAC
TCTATCCAGTTGTTTGGCCCAGGAATTCAGCCCCATCACTGTGACCTTACCAACATGGATGGAGTGGTCA
CTGTGACGCCCAGAAGTATGGACGCAGAACCTACGTGGAAAGGCCAGCGCATCTCAGAAACCACCATGCT
GCAGAGTGGCATGAAAGTGCAGTTTGGGGCGTCCCATGTATTTTGTTTGTTGGACCCCAGTCAGGATCAT
GCTCTTGCATAAAGATCTGTGGATGGAGGCCTGATGGTTAAGAGCCAAGACATAAAACCTGGAATTGTTC
AGGAGACAACTTTGATTTAGAGGAGATATTCATAGTGGGACAGCATTACCCACAAAGCAAGTAGCACCAC
TAGGCTGGACCGCGACAGAGTGTCGTCTGCCTCTAGCACAGCAGAGCGGGGAATGGTGAAGCCGATGATC
AGAGTAGAACAGCAGCCAGATTATCGCAGGCAAGAAAGCAGAACACAGGATGCTTCTGGGCCTGAGCTGA
TACTACCTGCAAAGCATTGTATTCAGGGAAGTTCTGAAGATTCATTTTTGTGTGCCATTATAAATTATAC
TAATAGCCTACAGTCCACTTTAAGTTTGTCCCCTACATATGTATTATATATGGCATGCCGGTATGTATTG
TCCAACCAGTACAGACCTGACATCAGCCCTACAGAGCGCACACATATGTGCATTGCAGTCGTCAACAAGA
TGGTGAGCATGATGGAGGGTGTCATCCAGAAAACAGAAGAATATTGCAGGGGCACTTGCTTCTGGATGGC
AAATGCATCTGAACTTCTCAACTTCATTAAGCAAGACCGAGACCTTAGTCGGATCACACTGGATGCTCAA
GATGTTTAGCACATTTGGTTTCAAATGGCATTTAAATACTTGGTTCACTGTCTTCAATCAGAACTTAATA
ATTACATGCCAGCCTTTCTAGATGACCCTGAAAGAGAAGTCTGCAACGACAATAGTATGGACTGGTCGCT
GCACACGOTCACAGGAGCCATGTCCTTGCTACGACGCTGCAGAGTCAATGCCGCCCTGACCATCCAGCTC
TTCTCTCAGCTCTCCACTTCATCAATATGTGGCTGTTCAATAGAAAGGTGGACCGACCCAGATTCGGGGC
TGTGCTCCCATTACTGGGGACGATTATCCGTCAGCAGTTGGGCCATATTGAAGGCCTGGGCTGAGAAGCA
GGGGCTGGAACTGGCTGCGGACTGTCATCTGAGCAGGATCGTGCAGGAAACGACTTGCTTAACCATGGAT
AAGTATGCACCTGATGACATTCCAAATATAAACAGCACCTGCTTTAAGTTAAATTCATTACAACTTCAAG
CCTTATTACAGAACTATCACTGTGCACCTGATGAGCCTTTTATCCCAACGGATCAAATAGAAAATGTAGT
GACTGTGGCTGAAAACACTGCCGATGAGCTGGCCCGCAGTGATGGAAGGGAAGTGCAGTTGGAGGAGGAT
CCTGATCTGCAGCTGCCGTTTCTTTTGCCAGAGATGGTTATTCTTGTGATGTTGTCAGAAAGCATTCCAA
ATGGTTTAGAGAATTTTTAGACCCTCTGTGCCAGAGAAGGATAATGAGGAAATTTCCTCACACACGTAAC
ACCAGGTACTTGGACAATATATTTTGAAGGTGCAGATTATGAAAGTCACCTTCTGCGTGAGAACACAGAG
CTGGCTCAGCCTCTGAGGAAAGAACCTGAAATAATCACTGTGACCCTAAAAAAGCAGAATGGAATGGGCC
TTAGCATAGTTGCAGCAAAGGGTGCTGGTCAAGATAAACTAGGAATCTACGTGAAGTCGGTTGTGAAAGG
AGGTGCTGCAGATGTGATGGACGTCTAGCTGAGGTGATCAGCTCGTCAGTGTGGATGGACAAGTCTCGGA
GTTGGACTCTCTCAGGAAAGGGCGGCAGAACTCATGACAAGAACAAGCTCTGTGGTGACACTGGAAGTAG
CAAAGCAGGGTGCCATCTACCACGGTAAGGCCACCCTTCTCAATCAGCCATCCCCCATGATGCAAAGAAT
TTCAGATCGTCGTGGCTCAGGTAAACCCCGACCAAAGAGTGAAGGCTTTGAGCTCTATAATAATTCAACT
CAAAATGGGTGTCCTGAGAGTCCTCAGCTGCCTTGGGCAGAATATAGTGAACCAAAGAAATTGCCTGGTG
ATGACAGAGGGATGAAAAATAGAGCTGATCACCGTTCCAGCCCCAACGTAGCAAATCAGCCTCCTAGTCC
TGGAGGGAAAAGTGCATATGCCTCTGGAACAACAGCGAAGATAACATCTGTCTCTACTGGAAACCTCTGC
ACTGAGGAGCAGACGCCTCCGCCTAGACCTGAAGCCTACCCCATTCCCAGTCAGACGTACAACAGAGAGT
ATTTTACCTTCCCAGCTTCCAAATCCCAGGATCGGATGGCTCCTCCTCAGAACCAGTGGCCAAATTATGA
GGAAAAGCCACATATGCACACAGATAGTAATCATTCCAGTATTGCAATTCAGCGTGTTACACGTTCCCAA
GAAGAACTTCGAGAAGATAAAGCTTACCAACTTGAGCGGCATCGAATAGAGGCAGCTATGGACCGAAAGT
CTGATAGTGATATGTGGATAAATCAGAGCTCCTCACTGGACTCCAGTACCTCTAGCCAGGAGCATCTGAA
CCATTCCTCTAAGTCGGTCACCCCTGCTTCCACACTGACCAAAAGTGGCCCTGGCCGTTGGAAAACACCA
GCAGCCATACCGGCCACCCCTGTGGCCGTCTCCCAGCCAATCCGAACAGACCTGCCTCCGCCACCCCCGC
CACCTCCAGTCCACTATGCCGGTGATTTCGATGGAATGTCCATGGATTTGCCTCTCCCACCACCCCGTTC
CGCCAACCAGATAGGGCTGCCGTCTGCGCAGGTGGCTGCTGCTGAACGGAGAAAGAGAGAAGAACATCAG
CGTTGGTATGAGAAGGAGAAGGCCCCCCTGGAGGAGGAGCGGGAGAGGAAGCGGAGAGAGCAGGAGAGGA
AGTTGGGCCAGATGCGCACTCAGTCCTTAAACCCTGCTCCGTTTTCTCCCCTGAGTGCACAGCAGATGAA
GCCCGAAAAGCCTTCCACACTCCAGCGGCCACAGGAAACAGTCATTCGGGAGCTGCAGCCTCAGCAGCAG
CCCCGCACGATCGAGCGCAGAGACTTGCAGTACATTACAGTCAGCAAAGAGGAGCTTTCCTCGGGGGACA
GTCTGTCCCCCGACCCGTGGAAGCGGGACGCCAAGGAGAAGCTGGAGAAGCAGCAGCAGATGCACATCGT
GGACATGCTGAGCAAGGAGATCCAGGAGCTCCAGAGCAAACCGGACCGCAGCGCCGAGGAGAGCGACCGG
CTGCGCAAGCTCATGCTGGAGTGGCAGTTCCAGAAGAGACTCCAGGAGTCGAAGCAGAAGGACGAAGATG
ACGAGGAGGAGGAGGACGATGATGTGGACACCATGGTGATCATGCAGCGCCTGGAGGCTGAACGAAGAGC
GAGGGTAAAGGGGGGAGTGCTTTGGCTGTGCCCATCTGTGGTCCCTATTTTAGCTTCTGCGTGTTTCCCA
TGGGGATAG
MLLT3
SEQ ID NO:90
>gi | 4758719 | ref | NM_004529.1 | Homo sapiens myeloid/lymphoid or mixed-
lineage leukemia (trithorax homolog, Drosophila); translocated to, 3
(MLLT3), mRNA
TTTGGGGCTGAGTTTAATAAGCGAGCGAGCGAGCAAGCGAGCGCGGGGGGAAAAAGGCAGAGAATGTCCG
CCATCTACCCTCCGCTCCTGGGCGCGCTCTCATTCATAGCAGCGTCTTCATGAATTACAGCTGAGGGGGG
GCGGAGGAGGGGGGGGTACCACACAACACCCCAGCAAACCTCCGGGCCCCCAGGCATGGCTAGGTCGTGT
TCCGTGCAGGTGAAGCTGGAGCTGGGGCACCGCGCCCAGGTGAGGAAAAAAACCCACCGTGGAGGCTTCA
CCCACGACTGGATGGTGTTCGTACGCGGTCCGGAGCACAGTAACATACAGCACTTTGTGGAGAAAGTCGT
CTTCCACTTGCACGAAAGCTTTCCTAGCCAAAAAGAGTGTGCAAAGATCCACCTTACAAAGTAGAAGAAA
TCTGGGTATGCTGGTTTCATTTTGCCAATTGAAGTTTATTTTAAAAACAAGGAAGAACCTAGGAAAGTCC
GCTTTGATTATGACTTATTCCTGCATCTTGAAGGCCATCCACCAGTGAATCACCTCCGCTGTGAAAAGCT
AACTTTCAACAACCCCACAGAGGACTTTAGGAGAAAGTTGCTGAAGGCAGGAGGGGACCCTAATAGGAGT
ATTCATACAGCAGCAGCAGCAGCAGCAGCAGTAGCAGCAGCACGCAGCAGCAGCAGCAGCAGCAGTAGCA
GCAGCAGCAGCAGCAGCAGCAGCAGCAGTAGCAGCAGCAGTAGCAGCAGCAGCAGCAGCAGTAGTACCAG
TTTTTCAAAGCCTCACAAATTAATGAAGGAGCACAAGGAAAAACCTTCTAAAGACTCCAGAGAACATAAA
AGTGCCTTCAAAGAACCTTCCAGGGATCACAACAAATCTTCCAAAGAATCCTCTAAGAAACCCAAAGAAA
ATAAACCACTGAAAGAAGAGAAAATAGTTCCTAAGATGGCCTTCAAGGAACCTAAACCCATGTCAAAGAA
GCCAAAACCAGATAGTAACTTACTCACCATCACCAGTGGACAAGATAAGAAGGCTCCTAGTAAAAGGCCG
CCCATTTCAGATTCTGAAGAACTCTCAGCCAAAAAAAGGAAAAAGAGTAGCTCAGAGGCTTTATTTAAAA
GTTTTTCTAGCGCACCACCACTGATACTCACTTGTTCTGCTGACAAAAAACAGATCAAAGATAAATCTCA
TGTCAAGATGGGAAAGGTCAAAATTGAAAGTGAGACATCAGAGAAGAAGAAATCAACGTTACCGCCATTT
GATGATATTGTGGATCCCAATGATTCAGATGTGGAGGAGAATATATCCTCTAAATCTGATTCTGAACAAC
CCAGTCCTGCCAGCTCCAGCTCCAGCTCCAGCTCCAGCCTTCAACCATCCCAGACCAGGCAACAAGGTCC
TTTGAGGTCTATAATGAAGATCTGCATTCTGATGACAATGAGGAGGAATCAGATGAAGTGGAGAATAACC
GACAATGACTCTGAAATGGAGAGGCCTGTAAATAGAGGAGGCAGCCGAAGTCGCAGAGTTAGCAAAAGTG
ATGGCAGCGATAGTGAAAGCAGTTCTGCTTCTTCACCCCTACATCACGAACCTCCACCACCCTTACTAAA
AACCAACAACAACCAGATTCTTGAAGTGAAAAGTCCAATAAAGCAAAGCAAATCAGATAAGCAAATAAAG
AATGGTGAATGTGACAAGGCATACCTAGATGAACTGGTAGAGCTTCACAGAAGGTTAATGACATTGAGAG
AAAGACACATTCTGCAGCAGATCGTGAACCTTATAGAAGAAACTGGACACTTTCATATCACAAACACAAC
ATTTGATTTTGATCTTTGCTCGCTGGACAAAACCACAGTCCGTAAACTACAGAGTTACCTGGAAACATCT
GGAACATCCTGAGGATATAACAACTGGATGCATCAAGAACTATTGTGTTTTTTTTTTTTGGTTTTTTTTT
TTTTTGGTTGTGATTTTTTGTTCTTGTTGTTTATATGAAAACACTCAAAATGATGCAACCAAAAGGGAAA
AAATAAAAATCAAACAACCTTCAGCTTTATTTTTCTTTAAAGCCAGTCATCATCTCTTGATAAAGGAGAG
GTTAAAGCAAACCAGCCTCAGCGGACCACTCTTCTCTCCAAGGAAATCCCCGGGAAGAGTTAGCCTGGAT
AGCCTTGAAAACAAACAAATCAAACACAACACAAGAAAACTCAAAGAATGTGTATGGTATCATGTATCTC
TCTGTGGTGGTTCATTTCCACAGGACGAATGCATATTCAACACACTGCCTTATTACATAACTGATCTATT
ATTATCGCATACAGATATTCTAAGTCGTTGAGGGAATGACACCATCAGACATTATAAGTACTTGGTCCCG
TGGATGCTCTTTCAATGCAGCACCCTTGCCATCCCAAGCCCAGTGACCTTACTCGTATACCGTGCCACTT
TCCACCAACTTTTTCCAAGTCCTTTAACTCGTTGCAGTCTGTATTTTCCACCTTTTGTTTTTCCAGTTCC
AGGACACAGATTATCAACTGGGGGGACCACCCTAGCCACCTTGATTTTCTTCTTTGTGGTCTTTTCCTGA
AAGTTGGGGCCCAGTCCTTGGCTGTATCCATGTAATGATCTTGGACCATGGTAGAAATGGCACCAAATAG
GATCATATGAATTGCTGTCTAGCCTTAGTCAATAAACTTGTAGGACTTTTAAACAAAAGTGTACCTGTAA
ATGTCCTGAATCCAGCATTGTTGAGCTGTCATCAACATTCTTGTGTCTGTTTTACTGTTACAATATTAGG
TGAATATGGAAGTAAAGGCATTCCACAGGATCATCATTTAAAAAAAAAGAATTCTGGTCCTGTTTTCTAA
AAAAAAAAAACTGTTGTAGAAATTCTTAATTTGGATCTATTTATTAGTCAGAGTTTCAGCTTTCTTCAGC
TGCCAGTGTGTTACTCATCTTTATCCTAAAAATCTGGAATCAGAGATTTTTGTTTGTTCACATATGATTC
TCTTAGACACTTTTATATTTGAAAAATTAAAATCTTTCTTTGGGGAAAAATTCTTGGTTATTGCTGCCAT
AACAGATTATGTAATTACTTGTAGATTCAGTGGTTCAATACCTGTTTAGTTGCTTGCTAATATTTCCAGA
AGGATTTCTTGTATTGGTGAAAGACGGTTGGGGATGGGGGGGATTTTTTTGTTCTTGTTGTACCCTTGTT
TGAAACTAGAAATCTGTCCTGTGGCATCAAAAGAAAGCAAATTATTTTTTAAAAGAAAAAAACCAAAGTA
CTTTTGGTGTCATTATTCCATCTTCTCCATAAGTGGAGAAATGAAAAGTAAGAACAGCTCATCTTCAAAG
TTTTTACTAGAAATTC
MLLT2
SEQ ID NO:91
>gi | 5174572 | ref | NM_005935.1 | Homo sapiens myeloid/lymphoid or mixed-
lineage leukemia (trithorax homolog, Drosophila); translocated to, 2
(MLLT2), mRNA
GGCAATTTCTTTTCCTTTCTAACTGTGGCCCGCGTTGTGCTGTTGCTGGGCAGGCGTTGGGCGCCGGCGG
TCTTCGAGCGTGGGGGCCCGCTGGCTTTCCCTTCTCAGAAACGCGCCGGGGGCGCTCGCTTGCCCCCGGA
TTCGGACGCGGCGCTCCCCGGGCTCGTCTGAAGTGCAGATCGCCGCAGAGGCCCCAGTGCCCGGATGTCC
ATCAGGATTAGCGCGAGCCAATACGGGCCGAGCCCGGGGCTGCGCCGAGGACGCCCGGGGCTCGAGAGCA
GGTAGTCCCGTAACATCGGGCGCCGCGCCGGGACGCGTCCCCGCCCGGCTCGCCAAATGGTGAGCGCGCG
GCGCTGGCAGCAGGGCCCGCGGGGTGAAGGCGCTCATGGACGGAAGACCCCTGGCTCTATAAGCTGAATT
ATGGCAGCCCAGTCAAGTTTGTACAATGACGACAGAAACCTGCTTCGAATTAGAGAGAAGGAAAGACGCA
ACCAGGAAGCCCACCAAGAGAAAGAGGCATTTCCTGAAAAGATTCCCCTTTTTGGAGAGCCCTACAAGAC
AGCAAAAGGTGATGAGCTGTCTAGTCGAATACAGAACATGTTGGGAAACTACGAAGAAGTGAAGGAGTTC
CTTAGTACTAAGTCTCACACTCATCGCCTGGATGCTTCTGAAAATAGGTTGGGAAAGCCGAAATATCCTT
TAATTCCTGACAAAGGGAGCAGCATTCCATCCAGCTCCTTCCACACTAGTGTCCACCACCAGTCCATTCA
CACTCCTGCGTCTGGACCACTTTCTGTTGGCAACATTAGCCACAATCCAAAGATGGCGCAGCCAAGAACT
GAACCAATGCCAAGTCTCCATGCCAAAAGCTGCGGCCCACCGGACAGCCAGCACCTGACCCAGGATCGCC
TTGGTCAGGAGGGTTCGGCTCTAGTCATCACAAGAAAGGTGACCGAAGAGCTGACGGAGACCACTTGTGC
TTCGGTGACAGATTCGGCTCCAGAGAGGGAGCTTTCTCCCTTAATCTCTTTGCCTTCCCCAGTTCCCCCT
TTGTCACCTATACATTCCAACCAGCAAACTCTTCCCCGGACGCAAGGAAGCAGCAAGGTTCATGGCAGCA
GCAATAACAGTAAAGGCTATTGCCCAGCCAAATCTCCCAAGGACCTAGCAGTGAAAGTCCATGATAAAGA
GACCCCTCAAGACAGTTTGGTGGCCCCTGCCCAGCCGCCTTCTCAGACATTTCCACCTCCCTCCCTCCCC
TCAAAAAGTGTTGCAATGCAGCAGAAGCCCACGGCTTATGTCCGGCCCATGGATGGTCAAGATCAGGCCC
CTAGTGAATCCCCTGAACTGAAACCACTGCCGGAGGACTATCGACAGCAGACCTTTGAAAAAACAGACTT
GAAAGTGCCTGCCAAAGCCAAGCTCACCAAACTGAAGATGCCTTCTCAGTCAGTTGACCAGACCTACTCC
AATGAAGTCCATTGTGTTGAAGAGATTCTGAAGGAAATGACCCATTCATGGCCGCCTCCTTTGACAGCAA
TACATACGCCTAGTACAGCTGAGCCATCCAAGTTTCCTTTCCCTACAAAGGACTCTCAGCATGTCAGTTC
TGTAACCCAAAACCAAAAACAATATGATACATCTTCAAAAACTCACTCAAATTCTCAGCAAGGAACGTCA
TCCATGCTCGAAGACGACCTTCAGCTCAGTGACAGTGAGGACAGTGACAGTGAACAAACCCCAGAGAAGC
CTCCCTCCTCATCTGCACCTCCAAGTGCTCCACAGTCCCTTCCAGAACCAGTGGCATCAGCACATTCCAG
CAGTGCAGAGTCAGAAAGCACCAGTGACTCAGACAGTTCCTCAGACTCAGAGAGCGAGAGCAGTTCAAGT
GACAGCGAAGAAAATGAGCCCCTAGAAACCCCAGCTCCGGAGCCTGAGCCTCCAACAACAAACAAATGGC
AGCTGGACAACTGGCTGACCAAAGTCAGCCAGCCAGCTGCCAGCCAGCTGCGCCACCAGGAGCAGGAGCC
CCCACGGCGGCACCCAGAGAGTAAGGGCAGCAGCGACAGTGCCACGAGTCAGGAGAATACTGAATCCAAA
GATCCTCCCCCTAAAGCTCCAGCAAAGCCCCCCGGGCCCCACCCGAGCCCCCCACCCCGCGGAAAGAGGA
GCTGTCAGAAGTCTCCGGCACAGCAGGAGCCCCCACAAAGGCAAACCGTTGGAACCAAACAACCCAAAAA
ACCTGTCAAGGCCTCTGCCCGGGCAGGTTCACGGACCAGCCTGCAGGGGGAAAGGGAGCCAGGGCTTCTT
CCCTATGGCTCCCGAGACCAGACTTCCAAAGACAAGCCCAAGGTGAAGACGAAAGGACGGCCCCGGGCCG
CAGCAAGCAACGAACCCAAGCCAGCAGTGCCCCCCTCCAGTGAGAAGAAGAAGCACAAGAGCTCCCTCCC
TGCCCCCTCTAAGGGTCTCTCAGGCCCAGAACCCGCGAAGGACAATGTGGAGGACAGGACCCCTGAGCAC
TTTGCTCTTGTTCCCCTGACTGAGAGCCAGGGCCCACCCCACAGTGGCAGCGGCAGCAGGACTAGTGGCT
GCCGCCAAGCCGTGGTGGTCCAGGAGGACAGCCGCAAAGACAGACTCCCATTGCCTTTGAGAGACACCAA
GCTGCTCTCACCGCTCAGGGACACTCCTCCCCCACAAAGCTTGATGGTGAAGATCACCCTAGACCTGCTC
TCTCGGATACCCCAGCCTCCCGGGAAGGGGAGCCGCCAGAGGAAAGCAGAAGATAAACAGCCGCCCGCAG
GGAAGAAGCACAGCTCTGAGAAGAGGAGCTCAGACAGCTCAAGCAAGTTGGCCAAAAAGAGAAAGGGTGA
AGCAGAAAGAGACTGTGATAACAAGAAAATCAGACTGGAGAAGGAAATCAAATCACAGTCATCTTCATCT
TCATCCTCCCACAAAGAATCTTCTAAAACAAAGCCCTCCAGGCCCTCCTCACAGTCCTCAAAGAAGGAAA
TGCTCCCCCCGCCACCCGTCCTCGTCCTCCCAGAAGCCAGCCAAGCCTGCACTTAAGAGGTCACAGGGCG
GGAAGCAGACACCTGTGGCCAGGACCCTCCCAAAAGTGCCAGCAGTACCAAGAGCAACCACAAAGACTCT
TCCATTCCCAAGCAGAGAAGAGTAGAGGGGAAGGGCTCCAGAAGCTCCTCGGAGCACAAGGGTTCTTCCG
GAGATACTGCAAATCCTTTTCCAGTGCCTTCTTTGCCAAATGGTAACTCTAAACCAGGGAAGCCTCAAGT
GAAGTTTGACAAACAACAAGCAGACCTTCACATGAGGGAGGCAAAAAAGATGAAGCAGAAAGCAGAGTTA
ATGACGGACAGGGTTGGAAAGGCTTTTAAGTACCTGGAAGCCGTCTTGTCCTTCATTGAGTGCGGAATTG
CCACAGAGTCTGAAAGCCAGTCATCCAAGTCAGCTTACTCTGTCTACTCAGAAACTGTAGATCTCATTAA
ATTCATAATGTCATTAAAATCCTTCTCAGATGCCACAGCGCCAACACAAGAGAAAATATTTGCTGTTTTA
TGCATGCGTTGCCAGTCCATTTTGAACATGGCGATGTTTCGTTGTAAAAAAGACATAGCAATAAAGTATT
CTCGTACTCTTAATAAACACTTCGAGAGTTCTTCCAAAGTCGCCCAGGCACCTTCTCCATGCATTGCAAG
CACAGGCACACCATCCCCTCTTTCCCCAATGCCTTCTCCTGCCAGCTCCGTAGGGTCCCAGTCAAGTGCT
GGCAGTGTGGGGAGCAGTGGGGTGGCTGCCACTATCAGCACCCCAGTCACCATCCAGAATATGACATCTT
CCTATGTCACCATCACATCCCATGTTCTTACCGCCTTTGACCTTTGGGAACAGGCCGAGGCCCTCACGAG
GAAGAATAAAGAATTCTTTGCTGGGCTCAGCACAAATGTGTGCACCTTGGCCCTCAACAGCAGTTTGGTG
GACCTGGTGCACTATACACGACAGGGTTTTCAGCAGCTACAAGAATTAACCAAAACACCTTAATGGAGCC
CCAGGTTGATTCAATGCCTTGGGAACTATTTTTGCACATTGGAAGCCTCAAAAACAGTCCAGACGTTTGT
TTCATCAGGACACCAAACTCTAAAAAAGAAGCACCACGAGATGGCCAGGACATTTGTCCACTTAAACTCT
CAACAACAGTGTGATCATTGGTTGGACACTGTGGTTATGCAGAAGCAGAGATGAGGAGGCTGGCCCCAGA
GATGATCTTGCCCTTCCTAACTAAAGGACAGAAGTGCAATTTAGCTTAAATGGGTGTATGAATGGTCTAG
AAACATTTCTATTTTTTTTTTAAACCAGCAGGATACAAGTTGCAAATGAAATGAGGAGAAACAGTTTCAA
GTCTGAAAGTGAATTTCACGTCATCTCAGTAGCCACGCTAGTCCATTCCCAGAAGGAAATTTTTTTTTTT
AACAATGACTTTTGGTAAAGGGTTTTGTGGATGATTTTTTTTCTTTTGAGTTTTGGGAGAAATATTTGTT
TAATAACTTCTAATGGCCATCTGTAAACCATAAGTAATGAAGGACTCCACTGTGCCCCACTTTCTGCCAA
TGAACAGTGGCTTGATAATACCAAGTATTGTTGTAATTTATAAAATTGAAGGCAACCCCCGCTCCTGCCG
CCCCCAATCTCCCCATTGCCTAGAGCGCTGCACATTGACCCCAGCTCTGACTTCTCATTACTGTGCTGAA
AGTCAGCCCACGTCGGAGCGGTGAGGAGGAGCCACAGCACATGGGGTGCCACCTCGAGGTCTGCACAGGA
GGACTTGGCGCTGCCATTTCCTACCCCTGCCATTTCCCACCCCTGCTTCAGCGAAAGGGACTCTCTAACA
GGGCAGTCACTGTTGACTCTATTCTGAATTTCCTCCCTTGGGGAAGAAGGGAACCAACATTTATACCTGA
CCAGATGGCTAAAGTGCTTTTAAAGTTTTGTTTAAGTAGAGCTGGAATTTGAGGTGCTGATCTGTGGTCT
ACAGTTATGTGGTAACTCATGTTGTCCAGCCAACTCAGAGTTTCGTCAGTGAACAAGAAACATGAAATCT
GCTTCTTAGAGAGGCTATATATTCTGCTACAAATATTTTATATTTTATAGCAAAACTAGACTTTCAGAGT
CCTTGATTGTCTAGGGGAAGTTAACTCCCTGAGAGGATGTAGAGATTTGGGGTGGTTGATTAGACTTTTG
AAAAACTCATCACCACATGCCTTCACTCCAAAAGTGTTCTCAGCTAGATTTGATTTGGTTGAGGAGGACT
AGTTCTTCTCCAGACTGTTCTTTGGTTGTCACTTAAGTCAGAGGTCTGGTCCCTCATGTTTAGGTGAAAG
TCTTTATGGCAGGTTCTATGCAGATTGTGCCAGAGCATGTGCGTGTTCTGTTGGCAAGCCACAGTGCTCC
CTCTTTGTGGGTTTTTTTTTTTCTCTGAACTTGATATAAGATTTTATTTGTCCGTTGAAAAAGTAAACAA
ATGTGCATAGATCAATTTGTACTACTTTGGTCATTGGATATTTCTGATCCTTATTGCATTGTACCTAAAG
GAGAGTAACTAATGGTAACCTTTTTAATAGAGTATGTGAAAGGTAGTGGCTGATGAATCCTTAACGTAAA
TAGGGTCTTTTTGCTGTTACGGAAGTATATAGAGGTCTGAAGGATTTTTAAAATGATTTGCACTTTTTCA
CTGCATGCTTACAATTCCCAAAGGCAAAATCTGTAGTGAGGTAGATCATTTGAAAGGGCTAGATTATAAA
ATTAAGCCTTAGAGTATGGAAAGTTCTTATAACAATAATAGTACACACTTCAGAGTAAGACAAATGCAAA
GCATCTTAAGGAGTGAAAATAGAGTCTAAATCTTGCCTTTGGCACTACAAGGTGTGTGTGTGTGTGTGTG
TTGTGTGTCTTTAGTAGGAAATGGAAGAACACTGTTTTATTTTTTAAAGTGTTTAATGTTTCTGTCCTTT
CTGTGAATTATTGAATTTAAGAGCCCTGCTAAATAATGAAAAAACACTAAACTAAAATTTATCAAATTAT
ACTGGGTTCGGATTGTGAAAACATTGGCCACCTAGTAGCAGTGGTGAGGAGTGGGAGGGCCCAGCAAGCA
TTTATCAGAAATAGAATCACAATAGGAGGAGAATTTGGCTGTCTGATATTATGATTTGATTACAATACTG
AATGGGAAAAGTATCTAATATTTTGTAACAAAAAGACCTTCATATTATCTGTTTTGACCAAAATATGTAG
CTATTTCCCTTACACAGATTGGACCGCACTTATCTCCCTTGTCCTGTATCGTTTAATTTCAGGTCTCAGG
ATGTTTAGAAAGCTAAAACCCCCTACCCCTTTCTGGCTGAAAACTTGCCTTATTTGGTATCTTACACATT
AATGTTACTAAGCATCAGGAGCTTACTGTTTTATTATGATTCATCTTAGTAATTTTTAGAAGCAAGAAGA
AAGCCATTGTGTCCTCTACAATTAACAAAACTTATCTCTGATATACAAAGGGATATAAATATATACACAA
AAATAGAGAAAAAGAGGTTGATTGAATTGTGCCTTTGAGTGAACCGAGTTTTTAAATACCGCTGTGTTTG
TTTCGCCATGGCTTCAGGGATGCTACATGGCTCTTGCACCTTTTACTCCTCTGCTTTATGAAGTTTGAGT
TGTATTTGTGCATCTTAAAGTAGGTTGAGGCTTGAGGCTGGGCTTTCGGGTTTTTTTGTTTTTTGTTTTG
TTTTGTTTTGTTTTGTTTTCTTGTACTTAAACCTGCTTGCTTCCTACCACAGATTCTTTATTTTCCCAAA
CACTACAAAAAAACTTTTAAAACTTTGCCATTTCATCTGTTTACAGTCTTTGCCACTGATTAGCAGTATT
TAAATCTTGCAAGAATATTTTGTGCTTTCTTTAGAAACACAAGAGTATAGATTTTTCTCACTGAAAAGTG
AGAGTTACGCATTGCAGCCATGAAGGGATGCTAGGATCAATTATGGCAGTACCTTTTTTCCCCTCCTGTT
CTTGAGCCAGTTGTCTCTTTTGTTTTGGGTCCCACTTAGGATTAACGGATGTAAGGTATTTTCCTGTGCC
TTTATTTTGTGTCATTCTATTGGAAGGAGGTGTAACGGCAGAATAGCATCGTGTTGGGGGTTTTCCTTCA
AACACTGCAAGTGATATTGCCACCATGTGAACCTCAAATATGCAATCCAGTTGTGTTGGTTTCTCGGTGA
CTTGGAGTGTTCATCTCTTCATGAATTGTGAGCACTGACCATGTTGTTCAGTTCTTAATTATGGTGAGTT
GACAAATACCAACTACTGCTTTTCTTTAGGTGGCTATAAATTTCTTACTGTCAGGAGGAAATGACATTAT
ATTCTGTTCCACTGAACGTCAGAGATCAGCAGGCACTGTACTGGGTAGAGAAGTGCCTATACTTCTCTAC
CTAAGAGGGCAGGAGGGAAACCCTACAGCTCCTTGTGAGCCTATATATTAGTATATCGGCCTGGAGAGGA
CAAGGGAATAAGACCACTCATAGTGAGGCTGGCCAAGCTGCACTGGTCGGACCAGGCAGTGGCTGACCTA
AGGAAGGCAACTTGCTTTGCTTAAAAGTAGATTTTTTAAGCAATGCTTAACACAGGCAGCATTCACCTTT
GTTCAGGCCATCGACATGTATTGTTAAAATTACTGCATATCCCCCTCAGATATCAAGTATACACTGTTCA
TGTTGGGGTTGTGTGTGTGTATGTGTGTATGTACGCACGCATGTGTCCCAAATCTTGTTTTAATTTTTTT
TTTCTGAATGTGATCATGTTTGGATAATACCTGAGCAGGGTTGCCTTTTTTATTATTTATTACCATTATA
TATTATATTATATTATATATTTTTTGCTTTCTTATAACTTTGGAGGAAAGTCAAATCTTGGTATTATTAA
AATTGTTTTAAAAAGGAGTAAATTTTCCAGTTGATAAATGAAAATCACTGGCCTATGTTTAATAAGTTTT
TCTTTAATTACTGTGGAATAACGTGCCAGCTATCATCAACACAATGATTTTGTACATAGGGTAGGGAAGC
AGTGATGCTCTCAATGGGAAGATGTGCAACACAAATTAAGGGGAACTCCATGTATTTTACCTACTTCAGC
AATGGAACTGCAACTTGGGGCTTTGTGAATAAAATTTAGCTGCCTTGTATAGTCGTTTGAAAGAATATGT
GATCTGTGAGAGAATTATAGTTTTTTTTTAGAAGAAAAATCTGCAAAAGATCTTTCCAAAGACAATGTGC
CACAGATCTTTTGTTCTCTGTAATGAGGATTAATTGCTGTTTAAACAAAAATGTAATTGTTCATCTTTAA
ATTCTTTCCTTTCATAAGAGGATCAAGCTGTAAAAAAACAAAAAAATTAATAAAAAATTTCGAGAAATCA
AAAAAAAAAA
MLLT10
SEQ ID NO:92
>gi | 4757725 | ref | NM_004641.1 | Homo sapiens myeloid/lymphoid or mixed-
lineage leukemia (trithorax homolog, Drosophila); translocated to, 10
(MLLT10), mRNA
GCCCTCTTGATTATGTGTGCCCTCTCCGGGCGCCCGCGTTAGCGGCCGGGTGGAGGTGGGGAGGGAAGAC
GCTGAGGAGGAGGAGGAGGCGGAGGAGGCGGTGGAGGGGACGTGGGGGGAATCAGCAAGGACATGGCTCC
TGAGTCCTGTGCGGAACGTGAGTGACTGAGCGGCAAAGCCCGAATGGTCTGTAGCGACCGGCCCGTGTCA
CTGGAGGACGAGGTCTCCCATAGTATGAAGGAGATGATTGGAGGCTGTTGCGTTTGCTCAGACGAGAGAG
GCTGGGCCGAGAACCCGCTGGTTTATTGCGACGGGCACGGCTGCAGCGTCGCGGTGCATCAAGCTTGCTA
TGGCATTGTTCAAGTACCCACTGGACCGTGGTTTTGCAGGAAATGTGAATCTCAGGAGAGAGCAGCCAGA
GTGAGATGTGAACTTTGTCCCCATAAGGATGGAGCTTTAAAAAGAACAGATAATGGGGGTTGGGCCCATG
TGGTTTGTGCCCTGTATATTCCAGAGGTACAATTTGCCAATGTTTCCACAATGGAACCAATTGTTTTACA
GTCTGTTCCGCATGATCGTTATAATAAGACTTGCTACATTTGTGATGAACAAGGAAGAGAAAGCAAAGCA
GCCACTGTGCTTGCATGACATGTAATAAAACATGGATGTCGACAGGCTTTCCATGTAACATGCGCTCAGT
TTGCCGGACTGCTTTGTGAAGAAGAAGGTAATGGTGCCGATAATGTCCAATACTGTGGCTACTGTAAATA
CCATTTTAGTAAGCTGAAAAAGAGCAAACGGGGATCTAATAGGTCATATGATCAAkGTTTAAGTGATTGT
TCCTCTCACTCTCAGGATAAACATCATGAGAAAGAGAAAAAAAAATATAAAGAGAAGGACPAACACAAAC
AGAAACACAAGAAGCAGCCAGAACCATCACCTGCATTGGTTCCATCCTTGACTGTTACTACAGAAAAAAC
TTATACAAGCACTAGCAACAACTCTATATCTGGATCATTGAAGCGCTTGGAAGATACTACTGCACGATTT
ACAAATGCAAATTTCCAGGAAGTCTCTGCACACACCTCTAGTGGAAAAGATGTTTCAGAGACTAGAGGGT
CAGAGGGCAAAGGGAAGAAATCTTCAGCTCACAGCTCAGGTCAAAGGGGAAGAAAGCCTGGTGGTGGAAG
AAATCCAGGAACAACTGTGTCAGCAGCTAGCCCTTTTCCTCAAGGCAGTTTTTCAGGAACTCCAGGCAGT
GTAAGTCAAATCTTCGGAAGTTCAGTGCAGTCTCCCCAGGATTTCCTGAGCTTTACAGACTCAGATCTGC
GTAATGACAGTTACTCTCACTCCCAACAGTCATCAGCAACCAAAGATGTACATAAAGGAGAGTCTGGAAG
CCAGGAAGGGGGGGTAAATAGTTTTAGTACCTTAATTGGCCTCCCTTCAACCTCAGCTGTTACCACACAG
CCTAAAAGCTTTGAAAATTCACCTGGAGATTTGGGTAATTCCAGCCTTCCTACAGCAGGATATAAGCGGG
CTCAAACTTCTGGCATAGAAGAAGAAACTGTAAAGGAAAAGAAAAGGAAAGGAAATAAACAAAGTAAGCA
TGGGCCTGGCAGACCCAAAGGAAACAAAAATCAAGAGAATGTTTCTCATCTCTCAGTTTCTTCTGCTTCA
CCAACATCATCTGTAGCATCAGCTGCAGGAAGCATAACAAGCTCTAGTATGCAGAAATCTCCTACATTGC
TCAGGAATGGAAGTTTACAGAGCCTCAGTGTTGGCTCATCTCCAGTTGGTTCACAAATTTCCATGCAGTA
TCGGCATGATGGAGCTTGCCCAACAACTACGTTCTCAGAGTTGCTGAATGCAATACACAACGACAGAGGT
GACAGTTCTACACTAACAAAGCAAGAACTTAAATTCATAGGTATTTATAACAGCAATGATGTAGCAGTAT
CGTTTCCAAATGTAGTATCTGGCTCGGGATCTAGTACTCCTGTCTCCAGCTCTCACTTACGTCAGCAGTC
TTCTGGGCATTTGCAACAAGTAGGAGCGCTCTCTCCCTCAGCTGTGTCATGTGCAGCCCCTGCTGTTGCT
ACAACTCAGGCAAATACTCTATCTGGATCTTCTCTCAGTCAGGCACCATCTCATATGTATGGCAATAGAT
CAAATTCATCAATGGCAGCTCTTATAGCTCAGTCTGAAAACAATCAAACAGATCAAGATCTTGGAGACAA
TAGCCGCAACCTAGTTGGCAGAGGAAGCTCACCCCGAGGAAGTCTCTCGCAACGATCCCCTGTAAGCAGC
TTACAGATTCGCTATGATCAACCAGGCAACAGCAGTTTGGAAAATCTGCCTCCAGTAGCAGCCAGCATAG
AACAGCTTTTGGAGAGGCAGTGGAGTGAAGGACAGCAATTTTTACTAGAACAGGGTACTCCTAGTGACAT
TTTAGGAATGCTGAAGTCATThCACCAACTTCAAGTTGAAAACCGAAGATTAGAGGAACAAATTAAAAAC
TTGAGTGCCAAAAAGGAACGGCTTCAGTTATTGAATGCACAGCTTTCAGTGCCTTTTCCAACAATAACAG
CAAATCCTAGTCCGTCTCATCAAATACACACATTTTCAGCACAGACTGCTCGTACTACTGATTCCTTGAA
CAGCAGTAAGAGCCCTCATATAGGAAACAGCTTTTTACCTGATAATTCTCTTCCTGTATTAAATCAGGAC
TTAACCTCCAGTGGACAAAGTACCAGCAGCTCATCAGCTCTTTCTACCCCACCTCCTGCTGGGCAGAGTC
CGGCTCAACAAGGCTCAGGAGTGACTGGAGTTCAGCAGGTCAATGGCGTGACAGTGGGGGCACTAGCTAG
GGAATGAGCCTGTAACTTCCACCATCCTGCCGTGTCGCAGTGGGTAGGAAATAATTAGGAGACTTTGCCA
GGTAACCAACTGGCAATTAATGGCATTGTAGGAGCTTTAAATGGGGTTATGCAGACTCCTGTCACAATGT
CCCAGAACCCTACCCCTCTCACCCACACAACCGTACCACCTAATGCAACACATCCAATGCCAGCTACACT
ACTACAGTGCCTAAAGGACTAGGAATTACTTTCTGACCAGCAACGACAAATACTTATTCATCAACAGCAG
TTTCAGCAGTTGTTAAATTCTCAACAGGTCACACCAGTACACAGGCACCCCCACTTCACACAGCTACCAC
CAACCCATTTCTCACCATCCATGGAGATAATGCAAGTCAGAAAGTAGCAAGACTTAGTGATAAAACTGGG
CCTGTAGCTCAAGAGAAAAGTTGACACCTGAGAAACATCTAGAAATTGCCTATCCTACTGTTCTAGCACT
TCATCTGGCTGCCTTTGCAGTCCTTTTACTACAGCTATGAAGAAACGCAACAAGAAACTCAATGCACAAC
AAAGGATTAATTGCTGCAAGGACATTCTTGTAAGGCTTTGATTAGTTTTCTTGTTGCTTTGTTGCACTGA
AATGGAATTCCCATGCCCCTACCCCTTACCCCAGTTTTTTGAACATGGAAAGAAAATTTAATAACTTTTT
AAAGTGACATAATTTACATGCAATATGTTTATCAACTCAAGAATTTAATATAGTTGGTACACAACTAGTT
TTGTTTATAAATTGGAGATGCAAATAGCAAAACTAAATACTTGCTCCATTTACAAACTACTTGATTTTAT
TGTACAAGTTGAAATATGCTCTTTGTTTGGGTTACAGTATCCTTGCTCTAAGTCAAATTCCAAGGAACTA
AATTTCTTCTCCTGGAGTTGCATTGATTCAGTATTACAAATATATAGCACATCACCTGGGAC
MLL2
SEQ ID NO:93
>gi | 4505196 | ref | NM_003482.1 | Homo sapiens myeloid/lymphoid or mixed-
lineage leukemia 2 (MLL2), mRNA
ATGGACAGCCAGAACCTGGCTGGTGAGGATAAAGATTCACAACCGGCAGCTGATGGACCTGCAGCCTCTG
AGGACCCAAGTGCCACTGAGTCAGACCTGCCCAACCCATGTGGGAGAGGTCTCTGTCCTTAGTTAACTGG
GAGTCCCAGGCTTCAGGAGACTCCTCAGGACTGCAGTGGGGGTCCGGTGCGGCGTTGTGCTCTCTGTAAC
TGCGGGGAGCCCGCTCTACACGGGCAGCGGGAGCTACGGCGCTTTGAGTTGCCATTTGATTGGCCCCGGT
GTCCAGTGGTGTCCCCTGGGGGGAGCCCAGGGCCAATGAGGCAGTGCTGCCCAGTGAGGACCTAATCACA
GATTGGTTTCCCTGAGGGCCTTACACCTGCCCACCTAGGAGAACCTGGAGGGTCCTGCTGGGCTCACCAT
TGGTGTGCTGCATGGTCGGCAGGCGTATGGGGGCAGGAGGGCCCACAACTATGTGGTGTGGACAAGGCCA
TCTTCTCAGGGATCTCACAGCGCTGCTCCCACTGCACCAGGCTCGGTGCCTCCATCCCTTGCCGCTCACC
TGGATGTCCACGGCTTTACCACTTCCCCTGCGCGACTGCCAGCGGTTCCTTCCTATCCATGAAAACACTG
CAGCTGCTATGCCCAGAGCACAGTGAGGGGGCTGCATATCTGGAGGAGGGTCGCTGTGCAGTGTGTGAGG
GGCCAGGGGAGCTGTGTGACCTGTTCTTCTGTACCAGCTGTGGGCATCACTATCACGGGGCCTGCCTGGA
CACTGCTCTGACTGCCCGCAAACGTGCTGGCTGGCAGTGCCCTGAATGCAAAGTGTGCCAAGCCTGCAGG
AAACCTGGGAATGACTCTAAGATGTTGGTTTGCGAGACGTGTGACAAAGGATACCATACTTTCTGCCTAA
AACCACCCATGGAGGAACTGCCTGCTCACTCTTGGAAGTGCAAGGCGTGCCGGGTGTGCCGGGCCTGTGG
GGCGGGCTCAGCAGAACTGAATCCCAACTCGGAGTGGTTTGAGAACTACTCTCTCTGTCACCGCTGTCAC
AAAGCCCAGGGAGGTCAGACCATCCGCTCCGTTGCTGAGCAGCATACCCCGGTGTGTAGCAGATTTTCAC
CCCCAGAGCCTGGCGATACCCCCACTGACGAGCCCGATGCTCTGTACGTTGCATGCCAAGGGCAGCCAAA
GGGTGGGCACGTGACCTCTATGCAACCCAAGGAACCAGGGCCCCTGCAATGTGAAGCCAAACCACTAGGG
AAAGCAGGGGTCCAACTTGAGCCCCAGTTGGAGGCCCCCCTAAACGAGGAGATGCCACTGCTGCCCCCAC
CTGAGGAGTCACCCCTGTCCCCACCACCTGAGGAATCACCCACGTCCCCACCACCTGAGGCATCACGCCT
GTCGCCACCACCTGAGGAATTGCCCGCATCCCCACTTCCTGAGGCATTGCACCTGTCCCGGCCGCTGGAG
GAATCGCCCCTCTCTCCGCCGCCTGAGGAGTCTCCTCTGTCTCCCCCACCTGTATCATCACCTTTTTGTC
CACTGGAGGAGTCGCCCTTGTCTCCACCGGAAGAGTCACCCCCATCTCCTGCACTTGAGACGCGTCTATC
CCCACCACCTGAAGCATCGCCCCTGTCCCCACCATTTGAAGAATCTCCTTTGTCCCCGCCACCTGAGGAA
TTGCCCACTTCCCCGCCACCTGAAGCATCTCGCCTGTCTCCACCACCTGAGGAGTCACCCATGTCCCCTC
CACGTGAAGAGTCACCCATGTCTCCACCACCGGAGGCATGTCGTCTGTTCCCACCATTTGAAGAGTCTCC
TCTGTCCCCTCCACCTGAGGAGTCTCCCCTTTCCCCACCACCTGAGGCATCACGCCTGTCCCCACCACCT
GAGGACTCGCCTATGTCCCCACCACGTGAAGAATCACCTATGTCCCCCCCACCTGAGGTATCGCGCCTAT
CCCCCCTGCCTGTGGTGTCACGCCTGTCTCCACCGCCTGAGGAATCTCCCTTGTCCCCACCGGCCCTGTC
TCCTTTGGGGGAGTTAGAGTACCCCTTTGGTGCCAAAGGGGACAGTGACCCTGAGTCACCGAAGGCTGCC
CCCATCCTGGAGACACCCATCAGCCCTCCACCAGAAGCTAACTGCACTGACCCTGAGCCTGTCCCCCCTA
TGATCCTTCCCCCATCTCCAGGCTCCCCAGTGGGGCCGGCTTCTCCCATCCTGATGGAGCCCCTTCCTCC
TCAGTGTTCGCCACTCCTTCAGCATTCCCTGGTTCCCCAAAACTCCCCTCCTTCCCAGTGCTGTCCTCCT
GCCCTACCACTGTCCGTTCCCACCCCGTTGAGTCCCATAGGGAAGGTAGTGGGGGTCTCAGATGAGGCTG
AGCTGCACGAGATGGAGACTGAGAAAGTTTCAGAACCTGAATGCCCAGCCTTGGAACCCAGTGCCACCAG
TCCTCTCCCTTCCCCAATGGGGGACCTTTCCTGCCCCGCCCCCAGCCCTGCCCCAGCCCTGGATGACTTC
TCTGGCCTAGGGGAAGACACAGCCCCTCTGGATGGGATTGATGCTCCGGGTTCACAGCCAGAGCCTGGAC
AGACCCCTGGCAGTTTGGCTAGTGAACTTAAAGGCTCCCCTGTGCTCCTGGACCCCGAGGAGCTGGCCCC
TGTGACCCCTATGGAGGTCTACCCCGAATGCAAGCAGACAGCAGGGCGGGGCTCACCATGTGAAGAACAG
GAAGAGCCACGTGCTCCGGTGGCCCCCACACCACCCACTCTCATCAAATCCGACATCGTTAACGAGATCT
CTAATCTGAGCCAGGGTGATGCCAGTGCCAGTTTTCCTGGCTCAGAGCCCCTCCTGGGCTCTCCAGACCC
GGAGGGGGGTGGCTCCCTGTCCATGGAGTTGGGGGTCTCTACGGATGTTAGTCCAGCCCGAGATGAGGGC
TCCCTACGGCTCTGTACTGACTCACTGCCAGAGACTGATGACTCACTATTGTGCGATGCTGGGACAGCTA
TCAGCGGAGGCAAAGCTGAGGGGGAGAAGGGGCGGCGGCGCAGCTCCCCAGCCCGTTCCCGCATCAAACA
GGGTCGCAGCAGCAGTTTCCCAGGAAGACGCCGGCCTCGTGGAGGAGCCCATGGAGGGCGTGGTAGAGGA
CGGGCCCGGCTAAAGTCAACTGCTTCTTCCATTGAGACTCTGGTAGTTGCTGACATTGATAGCTCTCCCA
GTAAGGAGGAGGAGGAAGAAGATGATGACACCATGCAGAATACCGTGGTTCTCTTCTCCAACACAGACAA
ATTTGTCCTAATGCAGGACATGTGTGTGGTATGTGGCAGCTTTGGCCGGGGGGCAGAGGGCCACCTCCTT
GCCTGTTCGCAGTGCTCTCAGTGCTATCACCCTTACTGTGTCAACAGCAAGATCACCAAGGTGATGCTGC
TCAAGGGCTGGCGTTGTGTGGAGTGTATTGTGTGTGAGGTGTGTGGCCAGGCCTCCGACCCCTCACGCCT
GCTGCTCTGTGATGACTGTGATATTAGCTACCACACATACTGCCTGGACCCCCCACTGCTCACCGTCCCC
AAGGGCGGCTGGAAGTGCAAGTGGTGTGTGTCCTGTATGCAGTGTGGGGCTGCTTCCCCTGGCTTCCACT
GTGAATGGCAGAATAGTTACACACACTGTGGGCCCTGTGCCAGCCTGGTGACCTGCCCTATCTGTCATGC
TCCTTACGTAGAAGAGGACCTACTAATCCAGTGCCGCCACTGTGAACGGTGGATGCATGCAGGCTGTGAG
AGCCTCTTCACAGAGGACGATGTGGACCACGCACCCGATGAAGGCTTTGACTGTGTCTCCTGCCAGCCCT
ACGTGGTAAAGCCTGTGGCGCCTGTTGCACCTCCAGAGCTGGTGCCCATGAAGGTGAAAGAGCCAGAGCC
CCAGTACTTTCGCTTCGAAGGCGTGTGGCTGACAGAAACTGGCATGGCCTTGCTGCGTAACCTGACCATG
TCACCACTGCACAAGCGGCGCCAACGGCGAGGACGGCTTGGCCTCCCAGGCGAGGCAGGATTGGAGGGTT
CTGAGCCCTCAGATGCCCTTGGCCCTGATGACAAGAAGGATGGGGACCTGGACACCGATGAGCTGCTCAA
GGGTGAAGGTGGTGTGGAGCACATGGAGTGCGAAATTAAACTGGAGGGCCCCGTCAGCCCTGATGTGGAG
CCTGGCAAAGAGGAGACCGAGGAAAGCAAAAAACGCAAGCGTAAACCATATCGGCCTGGCATTGGTGGTT
TCATGGTGCGACAGCGGAAATCCCACACACGCACGAAAAAGGGGCCTGCTGCACAGGGGGAGGTGTTGAG
TGGGGATGGCCAGCCCGACGAGGTGATACCTGCTGACCTGCCTGCAGAGGGCGCCGTGGAGCAGAGCTTA
GCTGAAGGGGATGAGAAGAAGAAGCAACAGCGGCGAGGGCGCAAGAGGAGCAAACTGGAGGGCATGTTCC
CTGCTTACTTGCAGGAAGCCTTCTTTGGGAAGGAGCTGCTGGACCTGAGCCGTAAGGCCCTTTTTGCAGT
TGGGGTGGGCCGGCCAAGCTTTGGACTAGGGACCCCAAAAGCCAAGGGAGATGGAGGCTCAGAAAGGAAG
GAACTCCCCACATCGCAGAAAGGAGATGATGGTCCAGATATTGCAGATGAAGAATCCCGTGGGCTCGAGG
GCAAAGCCGATACACCAGGACCTGAGGATGGGGGCGTGAAGGCATCCCCAGTGCCCAGTGACCCTGAGAA
GCCAGGCACCCCAGGTGAAGGGATGCTTAGCTCTGACTTAGACAGGATTTCCACAGAAGAACTGCCCAAG
ATGGAATCCAAGGACCTGCAGCAGCTCTTCAAGGATGTTCTGGGCTCTGAACGAGAACAGCATCTGGGTT
GTGGAACCCCTGGCCTAGAAGGCAGCCGTACGCCACTGCAGAGGCCCTTTCTTCAAGGTGGACTCCCTTT
GGGCAATCTGCCCTCCAGCAGCCCAATGGACTCCTACCCAGGCCTCTGCCAGTCCCCGTTCCTGGATTCT
AGGGAGCGCGGGGGCTTCTTTAGCCCGGAACCCGGTGAGCCCGACAGCCCCTGGACGGGCTCAGGTGGCA
CCACGCCCTCCACCCCCACAACCCCCACCACGGAGGGTGAGGGCGACGGACTCTCCTATAACCAGCGGAG
TCTTCAGCGCTGGGAGAAGGATGAGGAGTTGGGCCAGCTGTCCACCATCTCGCCTGTGCTCTATGCCAAC
ATTAATTTTCCTAATCTCAAGCAAGACTACCCAGACTGGTCAAGCCGTTGCAAACAAATCATGAAGGTCT
GGAGAAAGGTTCCAGCAGCTGACAAAGCCCCCTACCTGCAAAAGGCCAAAGATAACCGGGCAGCTCACCG
CATCAACAAGGTGCAGAAGCAGGCTGAGAGCCAGATCAACAAGCAGACCAAGGTGGGCGACATAGCCCGT
AAGACTGACCGACCGGCCCTACATCTCCGCATTCCCCCGCAGCCAGGGGCACTGGGCAGCCCGCCCCCCG
CTGCTGCCCCCACCATTTTCATTGGCAGCCCCACTACCCCCGCCGGCTTGTCTACCTCTGCGGACGGGTT
CCTGAAGCCGCCGGCGGGCTCGGTGCCTGGCCCTGACTCGCCTGGTGAGCTCTTCCTCAAGCTCCCACCC
CAGGTGCCCGCCCAAGCGCCTTCGCAGGACCCCTTTGGACTGGCCCCTGCCTATCCCCTGGAGCCCCGCT
TCCCCACGGCACCGCCCACCTATCCCCCCTATCCTAGTCCTACGGGGGCCCCTGCGCAGCCCCCGATGCT
GGGCGCCTCATCTCGTCCTGGGGCTGGCCAGCCAGGGGAATTCCACACTACCCCACCTGGCACCCCCAGA
CACCAGCCCTCCACACCTGACCCGTTCCTCAAACCCCGCTGCCCCTCGCTGGATAACTTGCCTGTGCCAA
AGAGCCCTGGGGTAGGGGGAGGCAGCAAACCGAGCCCCTGCTCTCGCCCCCACCTTTTTTTTGAGTCCAA
GAGGCCCTAGAGGTGAAGAAGGAGAGCTTGGGGATCCTTCCTAGCTATGGGCCCCCAAACCTAAGGCAAA
TTTGTTGACTCACCCTCCTCAGGCACCCACCTGGGTGGCCTGGAGTTAAAGACACCTGATGTCTTCAAAG
CCCCCCTGACCCCTCGGGCATCTCAGGTAGAGCCCCAGAGCCAGGGCTTGGGCCTAAGGCCCCAGGAGCC
ACCCCCTGCCCAGGCTTTGGCACCTTCTCCTCCAAGTCACCCAGACATCTTTCGCCCTGGCTCCTACAGT
GACCCATATGCTCAGCCCCCATTGACTCCTCGGCCCCAACCTCCGCCCCCTGAGAGCTGCTGTGCTCTGC
CCCCTCGCTCACTGCCCTCCGACCCTTTCTCCCGAGTGCCTGTCAGTCCTCAGTCCCAGTCCAGCTCCCA
GTCTCCACTGACACCCCGGCCTCTGTCTGCTGAAGCTTTTTGCCCATCACCCGTTACCCCTCGCTTCCAG
TCCCCGACCCTTATTCTCGCCCACCCTCACGCCGTCAGATCCCGTGACCCATTTGCCCCATTGCATAAGC
CACCCCGACCCCAGCCCCCTGAAGTTGCCTTTAAGGCTGGGTCTCTAGCCCACACTTCGCTGGGGGCTGG
GGGGTTCCCAGCAGCCCTGCCCGCGGGGCCAGCAGGTGAGCTCCATGCCAAGGTCCCAAGTGGGCAGCCC
CCCAATTTTGTCCGGTCCCCTGGGACGGGTGCATTTGTGGGCACCCCCTCTCCCATGCGTTTCACTTTCC
CTCAGGCAGTAGGGGAGCCTTCCCTAAAGCCCCCTGTCCCTCAGCCTGGTCTCCCGCCACCCCATGGGAT
CAACAGCCATTTTGGGCCCGGCCCCACCTAGGGCAAGCCTCAAAGCACAAAGTACACAGTAGCCACAGGG
AACTTCCACCCATCGGGCAGCCCCCTGGGGCCCAGCAGCGGGTCCACAGGGGAGAGCTATGGGCTGTCCC
CACTACGCCCTCCGTCGGTTCTGCCACCACGTGCACCCGACGGATCCCTCCCCTACCTGTCCCATGGAGC
CTCACAGCGATCAGGCATCACCTCTCCTGTCGAAAAGCGAGAAGACCCAGGGACTGGAATGGGTAGCTCT
TTGGCGACAGCTGAACTCCCAGGTACCCAGGACCCAGGCATGTCCGGCCTTAGCCAAACAGAGCTGGAGA
AGCAACGGCAGCGCCAGCGGCTACGAGAGCTGCTGATTCGGCAGCAGATCCAGCGCAACACCCTGCGGCA
GGAGAAGGAAACAGGTGCAGCAGCTGCGGGAGCAGTCGGGCCTCCAGGCAGCTGGGGTGCTGAGCCCAGC
AGCCCTGCCTTTGAGCAGCTGAGTCGAGGCCAGACCCCCTTTGCTGGGACACAGGACAAGAGCAGCCTTG
TGGGGTTGCCCCCAAGCAAGCTGAGTGGCCCCATCCTGGGGCCAGGGTCCTTCCCTAGCCATGACCGACT
CTCCCGGCCACCTCCACCAGCCACGCCTTCCTCTATGGATGTGAACAGCCGGCAACTGGTAGGAGGCTCC
CAAGCTTTCTATCAGCGAGCACCCTATCCTGGGTCCCTGCCCTTACAGCAGCAACAGCAACAACTGTGGC
AGCAACAACAGGCAACAGCAGCAACCTCCATGCGATTTGCCATGTCAGCTCGCTTTCCATCAACTCCTGG
ACCTGAACTTGGCCGCCAAGCCCTAGGTTCCCCGTTGGCGGGAATTTCCACCCGTCTGCCAGGCCCTGGT
GAGCCAGTGCCTGGTCCAGCTGGTCCTGCCCAGTTCATTGAGCTGCGGCACTTTGTACAGAAAGGACTGG
GACCTGGGGGCACTCCGTTTCCTGGTCAGGGCCCACCTCAGAGACCCCGTTTTTACCCTGTAAGTGAGGA
CCCCCACCGACTGGCTCCTGAAGGGCTTCGGGGCCTGGCGGTATCAGGTCTTCCCCCACAGAAACCCTCA
GCCCCACCGGCCCCTGAAATGAACAACAGTCTTCATCCAACACCCCACACCAAGGGTCCTACCCTGCCAA
CTGGTTTGGAGCTGGTCAACCGGCCCCCGTCGAGCACTGAGCTTGGCCGCCCCAATCCTCTGGCCCTGGA
AGCTGGGAAGTTGCCCTGTGAGGATCCCGAGCTGGATGACGATTTTGATGCCCACAAGGCCCTAGAGGAT
GATGAAGAGCTTGCTCACCTGGGTCTGGGTGTGGATGTGGCCAAGGGTGATGATGAACTTGGCACCTTAG
AAAACCTGGAGACCAATGACCCCCACTTGGATGACCTGCTCAATGGAGACGAGTTTGACCTGCTGGCATA
TACTGATCCTGAGCTGGACACTGGGGACAAGATGGATATCTTCAATGAGCACCTGAGGCTGGTAGAATCG
GCTAATGAGGAGGCTGAACGGGAGGCCCTGCTGCGGGGGGTGGAGCCACGACCCTTGGGCCCTGAGGAGC
GCCCTCCCCCTGCTGCTGATGCCTCTGAACCCCGCCTGGCATCTGTGCTCCCTGAGGTGAAGCCCAAGGT
GGAGGAGGGTGGACGCCACCCTTCTCCTTGCCAATTCACCATTGGTACCCCCAAGGTAGAGCCCGCACCT
GCTGCCAATTCCCTTGGCCTGGGGCTAAAGCCAGGACAGAGCATGATGGGCAGCCGGGATACCCGGATGG
GCACAGGGCCATTTTCTAGCAGTGGGCACACAGCTGAGAAGGCCTCCTTTGGGGCCACGGGAGGGCCACC
AGCTCACCTGCTGACCCCCAGCCCACTGAGTGGCCCAGGAGGATCCTCCCTGCTGGAAAAGTTTGAGCTC
GAGAGTGGGGCTTTGACCTTGCCTGGTGGACCTGCAGCATCTGGGGATGAGCTAGACAAGATGGAGAGCT
CACTGGTAGCCAGCGAGTTACCCCTGCTCATTGAGGACCTGTTGGAGCATGAGAAGAAGGAGCTGCAGAA
GAAGCAGCAGCTTTCAGCACAGTTGCAGCCTGCCCAGCAGCAGCAGCAACAGCAGCAGCAGCATTCCCTA
CTGCCTGCACCAGGCCCTGCCCAGGCCATGTCTTTGCCACATGAGGGCTCTTCTCCCAGTTTGGCTGGGT
CCCAACAGCAGCTTTCCCTGGGTCTTGCAGTTGCCCGACAGCCAGGTTTGCCCCAGCCACTGATGCCCAC
CCAGCCACCAGCTCATGCCCTCCAGCAACGCCTGGCTCCATCCATGGCTATGGTGTCCAATCAAGGGCAT
ATGCTAAGTGGGCAGCATGGAGGGCAGGCAGGCTTGGTACCCCAGCAGAGCTCACAGCCAGTGCTATCAC
AGAGCCCATGGGCACCATGCCACCTTCCATGTGCATGAAGCCGCAGACAATTGGCAATGCAGCAGCAGCT
GGCAAACIGCTTCTTCCCAGATACAGACCTGGACAAATTTGCTGCAGAAGATATCATTGGTCCCATTGCA
AAGGCCAAGATGGTGGCTTTGAAAGGCATCAAGAAAGTGATGGCTCAGGGCAGCATTGGGGTGGCACGTG
GTATGAACAGACAGCAAGTGTCTCTGCTAGCCCAGAGGCTCTCGGGGGGACCTAGCAGTGATCTGCAGAA
CCATGTGGCAGCTGGGAGTGGCCAGGAGCGCCAAGTGCTGGTGATCCCTCCAGCTCGTCCCAACCCGCCC
ACTTTTGCTCAGGGAGTGATCAATGAAGCTGACCAGCGGCAGTATGAGGAGTGGCTGTTCCATACCCAGC
AGCTCCTACAGATGCAGCTGAAGGTGCTAGAGGAGCAGATTGGTGTAACCGAAGTCCCGGAAAAGGCTCT
GTGTGCCAAGCAGCGCACTGCCAAAAAAGCTGGCCGTGAGTTCCCAGAAGCTCATGCTGAGAAGCTCAAG
CTGGTTACAGAGCAGCAGAGCAAGATCCAGAAACAACTGGATCAGGTACGGAAACAGCAGAAGGAGCACA
CTAATCTCATGGCAGAATATCGGAACAAGCAGCAAGCAACAACAGCAGAGCAGCACCAACAACAGCAACA
GCACTCAGCTGTGCTGGCTCTCAGCCCTTCCCAGAGTCCCCGGCTGCTCACCAAGCTCCCTGGTCAGCTG
CTCCCTGGCCATGGGCTGCAGCCACCACAGGGGCCTCCGGGTGGGCAAGCCGGAGGTCTTCGCCTGACCC
CTGGGGGTATGGCACTACCTGGACAGCCTGGTGGCCCCTTCCTTAATACAGCTCTGGCCCAACAGCAGCA
ACAGCAACATTCTGGTGGGGCTGGATCCCTGGCTGGCCCTTCAGGGGGCTTGTTCCCTGGCAACCTTGCT
CTTCGAAGCCTCGGACCTGATTCAAGGCTTTTACAGGAAAGGCAGCTGCAGCTGCAGCAGCAACGTATGC
AGCTGGCCCAGAAACTGCAGCAGCAGCAGCAGCAGCAACAGCAGCAGCAGCACCTTCTAGGACAGGTGGC
AATCCAGCAGCAACAGCAGCAGGGTCCTGGAGTACAGACAAACCAAGCTCTGGGTCCCAAGCCCCAGGGC
CTTATGCCTCCCAGCGCCACCAAGGCCTCCTGGTCCAGCAGCTGTCCCCTCAACCACCCCACGAGGCCCC
AGGGCATGCTGGGCCCTGCCCAGGTGGCTGTGTTGCAGCAGCAGCACCCTGGAGCTTTGGGCCCCCAGGG
CCCTCACAGACAGGTGCTTATGACCAGTCCCGGGTGCTGAGTTCCCCCCAGCTGGCACAGCAGGAGTCAG
GGCCTTATGGGACACAGGCTGGTCACAGCCCAGCAGCAGCAGCAGCAACAACAGCACCAACAGCAAGGGT
CCATGGCAGGGCTGTCCCATCTTCAGCAAAGTCTGATGTCACACAGTGGGCAGCCCAAACTGAGCGCTCA
GCCCATGGGCTCTTTACAGCAGCTTCAGCAGCAGCAGCAGCTGCAACAGCAACAGCAACTTCAGCAGCAG
CAGCAGCAGCAGCTACAACAGCAACAGCAACTTCAGCAGCAACAGCTTCAACAGCAGCAACAGCAGCAGC
AGCTTCAACAACAGCAGCAGCAACAGCTTCAACAGCAGCAACAGCAGCTACAACAGCAACAGCAACAACA
ACAGCAGCAGTTTCAACAGCAGCAGCAACAGCAGCAGCTGGCCTTTTAAACCAGATCGAACTTTACATGA
TCCCCTCAGCAACAACAGCAGCAGCAAGTGGCACTTGGCCCTGGCATGCCAGCAAAGCCTCTTCAACACT
TTTCTAGCCCTGGAGCCCTGGGTCCACCCTCCTCCTGACGGGCAAGGAACAAAACACCGTAGACCCAGCA
CGTTTCTTCAGAGGCCACTGAGGGGCCCTCTACACATCAGGGAGGGCCGTTAGCAATAGGAACTACCCCT
GAGTCAATGGCCACTGAACCAGGAGAGGTAAAGCCCTCACTCTCTGGGGACTCACAACTCCTGCTTGTCC
AACCCCAGCCCCAGCCTCAGCCCAGCTCTCTGCAGCTGCAGCCACCTCTGAGGCTTCCAGGACAACAGCA
GCAGCAAGTTAGCCTGCTCCACACAGCAGGTGGAGGAAGCCATGGGCAGCTAGGCAGTGGATCATCTTCT
GAGGCCTCATCTGTGCCCCACCTGCTGGCTCAGCCCTCTGTTTCCTTAGGGGATCAGCCTGGGTCCATGA
CCCAGAACCTTCTGGGCCCCCAACAGCCCATGCTAGAGCGGCCCATGCAAAATAATACAGGGCCACAACC
TCCCAAACCAGGACCTGTCCTCCAGTCTGGGCAGGGTCTGCCTGGGGTTGGAATCATGCCTACGGTGGGT
CAGCTTCGAGCACAGCTCCAAGGAGTCCTGGCCAAAAACCCACAGCTGCGGCACTTAAGTCCTCAGCAGC
AGCAGCAGCTACAGGCACTCCTCATGCAGCGGCAGCTGCAGCAGCAGTCAGGCAGTACGCCAGACCCCAC
CTACCAGGAGCCTGGGACCCAGACCTCTCCCCTCCAGGGCCTCCTGGGCTGCCAACCTCAACTTGGGGGC
TTCCCTGGACCACAGACAGGCCCCCTCCAGGAGCTAGGGGCAGGGCCTCGACCTCAGGGCCCACCCCGGC
TCCCTGCCCCACCAGGAGCCTTATCTACAGGACCAGTCCTTGGCCCTGTCCATCCCACACCTCCACCATC
CAGCCCTCAAGAGCCAAAGAGACCTTCACAATTACCTTCCCCCAGCTCCCAGCTTCCCACTGAGGCCCAG
CTCCCTCCCACCCATCCAGGGACCCCCAAACCTCAGGGGCCAACCTTGGAGCCGCCTCCTGGGAGGGTCT
GACCTGCTGCTGCCCAGCTTGCAGATACCTTGTTTAGCAAGGGTCTGGGACCTTGGGATCCCCCAGACAA
CCTAGCAGAAACCCAGAAGCCAGAGCAGAGCAGCCTGGTACCTGGGCATCTGGACCAGGTGAATGGACAG
GTGGTGCCTGAGGCATCCCAACTCAGCATCAAGCAGGAACCTCGGGAAGAGCCATGTGCCCTGGGAGCCC
AGTCAGTGAAGAGGGAGGCCAATGGGGAGCCAATAGGGGCACCAGGAACCAGAAACCACCTCCTGCTGGC
AGGCCCTCGCTCAGAAGCTGGGCATCTGCTCTTGCAGAAGCTACTCCGGGCAAAGAATGTGCAACTCAGC
ACTGGGCAGGGGTCCGAGGGGCTGCGAGCTGAGATCAACGGGCACATTGACAGCAAGCTGGCTGGGCTGG
AGCAGAAACTACAGGGTACCCCCAGCAACAAGGAGGATGCAGCAGCAAGGAAGCCTTTGACACCGAAGCC
CAAGCGGGTACAGAAGGCAAGCGACAGGTTGGTGAGCTCCCGAAAGAAGCTGCGGAAGGAGGACGGCGCT
AGGGCCAGCGAGGCCTTGCTGAAACAGCTGAAACAGGAGCTGTCCCTGCTGCCCCTAACGGAGCCTGCTA
TCACCGCCAATTTTAGCCTCTTTGCCCCCTTTGGCAGTGGCTGCCCAGTCAATGGGCAGAGCCAGCTGAG
GGGGGCCTTTGGAAGTGGGGCGCTGCCCACTGGCCCTGACTACTATTCCCAGCTGCTTACCAAGAATAAC
CTGAGTAACCCGCCGACACCACCCTCGTCGCTGCCCCCCACCCCACCCCCATCGGTGCAGCAGAAGATGG
TGAATGGCGTCACCCCATCTGAAGAGCTGGGGGAGCACCCCAAGGATGCTGCCTCTGCCCGGGATAGTGA
AAGGGCACTGAGGGATACTTCAGAGGTGAAGAGTCTAGACCTGCTTGCTGCCTTGCCTACACCCCCTCAC
AATCAGACTGAGGATGTCAGGATGGAGAGTGATGAGGATAGCGATTCTCCTGACAGCATTGTGCCAGCTT
CATCCCCTGAGAGCATCTTGGGGGAGGAGGCCCCCTCGTTTCCCTCATCTGGGCTCAGGCCGGTGGGAGC
AGAGGACCGGGCCCTCTCCCCTGTCATCCCCCTCATTCCTCGGGACAGCATCCCAGTCTTCCCAGATACC
AAACCTTATGGGGCCCTTGGCCTGGAGGTCCCTGGAAAGCTGCCTGTCACAACTTGGGAAAAGGGCAAAG
GAAGTGAGGTGTCAGTCATGCTCACAGTCTCTGCTGCTGCAGACAAGAACCTGAATGGCGTGATGGTGGC
AGTGGCGGAGCTGCTGAGCATGAAGATCCCCAACTCCTATGAGGTGCTGTTCCCAGAGAGCCCCGCCCGG
GGAGGCACTGAGCCAAAGAAGGGGGAAGCTGAGGGTCCTGGTGGGAAGGAAAAGGGTCTGGAAGGCAAGA
GCCCAGACACTGGCCCTGATTGGCTGAAGCAGTTTGATGCAGTGTTGGCTGGCTATACCCTGAAGAGGCA
ACTAGACATCTTGAGCCTCCTGAAACAGGAGAGCCCCGCCCCAGAGCCACCCACTCAGCACAGGTATACC
TACAATGTCTCCAATCTGGATGTGCGACAGCTCTCGGCCCCACCTCCTGAAGAACCCTCCCCGCCCCCTT
CCCCCTTGGCACCTTCTCCTGCCAGTCCCCCTACTGAGCCCTTGGTTGAACTTCCCACCGAACCCTTGGC
TGAGCCACCCGTCCCCTCACCTCTGCCACTGGCCTCATCCCCTGAATCAGCCCGACCCAAGCCCCGTGCC
CGGCCCCCTGAAGAAGGTGAAGATACCCGTCCTCCTCGCCTCAAGAAATGGAAAGGAGTGCGCTGGAAGC
GGCTTCGGCTGCTGCTGACCATCCAGAAGGGCAGTGGACGGCAGGAGGATGACGGGAAGTGGCAGAGTTA
TATGGAGCAGCTTGGCACAGCCTTGCGACCTGACAAGGTACCGCGAGACATGCGTCGCTGCTGTTTCTGT
CATGAGGAGGGTGACGGGGCCACTGATGGGCCTGCCCGTCTGCTGAACCTGGACCTGGACCTGTGGGTGC
ACCTCAACGATGCCCTTTGGTCCACGGAGGTGTATGAGACCCAGGGCGGAGCACTGATGAATGTGGAGGT
TGCCCTGCACCGAGGACTGCTACCAAGTGCTCCCTGTGCCAGCCAAAGTCGCGCGACCAGCAGCTGAAAT
CGCATGCGTTGCCCCATGTCTACCATTTTGGTTGTGCCATCCGCGCAAGTGCATGTTCTTAGATCACTAA
AGACCATGCTGTGTCCAATGCATAAGATCAAGGGGCCCTGTGAGCAAGAGCTGAGCTCTTTTGCTGTCTT
CCGGCGGGTCTACATTGAGCGGGACGAGGTGAAGCAAATCGCTAGCATCATTCAGCGGGGAGAACGGCTG
CACATGTTCCGTGTGGGGGGGCTTGTGTTCCACGCCATCGGACAGCTGCTGCCTCACCAGATGGCTGACT
TTCATAGTGCCACTGCCCTCTATCCCGTGGGCTACGAGGCCACGCGCATCTATTGGAGCCTCCGCACCAA
CAATCGTCGCTGCTGCTATCGCTGTTCTATTGGTGAGAACAACGGGCGGCCGGAGTTTGTAATCAAAGTC
ATCGAGCAGGGCCTGGAGGACCTGGTCTTCACTGACGCCTCTCCCCAGGCCGTGTGGAATCGCATCATTG
AGCCTGTGGCTGCCATGAGAAAAGAGGCTGACATGCTGCGACTCTTCCCTGAGTATCTGAAGGGCGAGGA
GCTCTTTGGGCTGACGGTGCATCCGTGCTTCGCATAGCTGAATCACTGCCCGGGGTGAGAGACTGGTCAA
AACTATTTATTCCGCTATGGGCGCCACCCCCTTATGGAGCTGCCACTCATGATCAACCCCACTGGCTGTG
CCCGATCAGAGCCTAAAATCCTCACACACTACAAACGGCCCCATACCCTGAACAGCACCAGCATGTCTAA
GGCATATCAGAGCACCTTCACAGGCGAGACCAACACCCCCTACAGCAAGCAGTTTGTGCACTCCAAGTCA
TCTCAGTACCGGCGGCTGCGCACCGAATGGAAGAACAACGTGTACCTGGCTCGCTCCCGTATCCAGGGCC
GGGGCTCTATGCAAGCCAAGGACCTAGAAAAGCACACAATGGTTATCGAGTACATTGGCACCATCATTCG
GAACGAGGTGGCCAACCGGCGGGAGAAAATCTACGAAGAGCAGAATCGAGGCATCTACATGTTCCGAATA
ACAATGAACATGTGATTGATGCTACGTTGACCGGCGGCCCTGCACAGGTACATTAACCATTCCTGTGCCC
CTAACTGTGTGGCGAAAGTCGTGACATTTGACAAAGAGGACAAAATCATCATCATCTCCAGCCGGCGAAT
CCCCAAGGAGAGGAGCTAACCTATGAGTATCAGTATTGATTTTGAGGACGATCAGCACGAGATCCCCTGC

TABLE 4

Histone Deacetylases

HDAC1
SEQ ID NO:94
>gi | 13128859 | ref | NM_004964.2 | Homo sapiens histone deacetylase 1 (HDAC1),
mRNA
GAGCGGAGCCGCGGGCGGGAGGGCGGACGGACCGACTGACGGTAGGGACGGGAGGCGAGCAAGATGGCGC
AGACGCAGGGCACCCGGAGGAAAGTCTGTTACTACTACGACGGGGATGTTGGAAATTACTATTATGGACA
AGGCCACCCAATGAAGCCTCACCGAATCCGCATGACTCATAATTTGCTGCTCAACTATGGTCTCTACCGA
AAAAATGGAATCTATCGCCCTCACAAAGCCAATGCTGAGGAGATGACCAAGTACCACAGCGATGACTACA
TTAAATTCTTGCGCTCCATCCGTCCAGATAACATGTCGGAGTACAGCAAGCAGATGCAGAGATTCAACGT
TGGTGAGGACTGTCCAGTATTCGATGGCCTGTTTGAGTTCTGTCAGTTGTCTACTGGTGGTTCTGTGGCA
AGTGCTGTGAAACTTAATAAGCAGCAGACGGACATCGCTGTGAATTGGGCTGGGGGCCTGCACCATGCAA
AGAAGTCCGAGGCATCTGGCTTCTGTTACGTCAATGATATCGTCTTGGCCATCGTGGAACTGCTAAAGTA
TCACCAGAGGGTGCTGTACATTGACATTGATATTCACCATGGTGACGGCGTGGAAGAGGCCTTCTACACC
ACGGACCGGGTCATGACTGTGTCCTTTCTTAAGTATGGAGAGTACTTCCCAGGAACTGGGGACCTACGGG
ATATCGGGGCTGGCAAAGGCAAGTATTATGCTGTTAACTACCCGCTCCGAGACGGGATTGATGACGAGTC
CTATGAGGCCATTTTCAAGCCGGTCATGTCCAAAGTAATGGAGATGTTCCAGCCTAGTGCGGTGGTCTTA
CAGTGTGGCTCAGACTCCCTATCTGGGGATCGGTTAGGTTGCTTCAATCTAACTATCAAAGGACACGCCA
AGTGTGTGGAATTTGTCAAGAGCTTTAACCTGCCTATGCTGATGCTGGGAGGCGGTGGTTACACCATTCG
TAACGTTGCCCGGTGCTGGACATATGAGACAGCTGTGGCCCTGGATACGGAGATCCCTAATGAGCTTCCA
TACAATGACTACTTTGAATACTTTGGACCAGATTTCAAGCTCCACATCAGTCCTTCCAATATGACTAACC
AGAACACGAATGAGTACCTGGAGAAGATCAAACAGCGACTGTTTGAGAACCTTAGAATGCTGCCGCACGC
ACCTGGGGTCCAAATGCAGGCGATTCCTGAGGACGCCATCCCTGAGGAGAGTGGCGATGAGGACGAAGAC
GACCCTGACAAGCGCATCTCGATCTGCTCCTCTGACAAACGAATTCCCTGTGAGGAAGAGTTCTCCGATT
CTGAAGAGGAGGGAGAGGGGGGCCGCAAGAACTCTTCCAACTTCAAAAAAGCCAAGAGAGTCAAAACAGA
GGATGAAAAGAGAAAAGACCCAGAGGAGAAGAAAGAAGTCACCGAAGAGGAGAAAACCAAGGAGGAGAAG
CCAGAAGCCAAAGGGGTCAAGGAGGAGGTCAAGTTGGCCTGATTGGACCTCTCCAGCTCTGGCTTCCTGC
TGAGTCCCTCACGTTTCTTCCCCATCCCCTCAGATTTTATATTTTCTATTTCTCTGTGTATTTATATAAA
AATTTATTAAATATAAATATCCCCAGGGACAGAAACCAAGGCCCCGAGCTCAGGGCAGCTGTGCTGGGTG
AGCTCTTCCAGGAGCCACCTTGCCACCCATTCTTCCCGTTCTTAACTTTGAACCATAAAGGGTGCCAGGT
CTGGGTGAAAGGGATACTTTATGCAACCATAAGACAACTCCTGAAAAATGCCAAGTGCCTGCTTAGTAGC
TTTGGAAAGGTGCCCTTATTGAACATTCTAGAAGGGGTGGCTGGGTCTTCAAGGATCTCCTGTTTTTTTC
AGGCTCCTAAAGTAACATCAGCCATTTTTAGATTGGTTCTGTTTTCGTACCTTCCCACTGGCCTCAAGTG
AGCCAAGAAACACTGCCTGCCCTCTGTCTGTCTTCTCCTAATTCTGCAGGTGGAGGTTGCTAGTCTAGTT
TCCTTTTTGAGATACTATTTTCATTTTTGTGAGCCTCTTTGTAATAAAATGGTACATTTCT
HDAC2
SEQ ID NO:95
>gi | 4557640 | ref | NM_001527.1 | Homo sapiens histone deacetylase 2 (HDAC2), mRNA
CGCCGAGCTTTCGGCACCTCTGCCGGGTGGTACCGAGCCTTCCCGGCGCCCCCTCCTCTCCTCCCACCGG
CCTGCCCTTCCCCGCGGGACTATCGCCCCCACGTTTCCCTCAGCCCTTTTCTCTCCCGGCCGAGCCGCGG
CGGCAGCAGCAGCAGCAGCAGCAGCAGGAGGAGGAGCCCGGTGGCGGCGGTGGCCGGGGAGCCCATGGCG
TACAGTCAAGGAGGCGGCAAAAAAAAAGTCTGCTACTACTACGACGGTGATATTGGAAATTATTATTATG
GACAGGGTCATCCCATGAAGCCTCATAGAATCCGCATGACCCATAACTTGCTGTTAAATTATGGCTTATA
CAGAAAAATGGAAATATATAGGCCCCATAAAGCCACTGCCGAAGAAATGACAAAATATCACAGTGATGAG
TATATCAAATTTCTACGGTCAATAAGACCAGATAACATGTCTGAGTATAGTAAGCAGATGCATATATTTA
ATGTTGGAGAAGATTGTCCAGCGTTTGATGGACTCTTTGAGTTTTGTCAGCTCTCAACTGGCGGTTCAGT
TGCTGGAGCTGTGAAGTTAAACCGACAACAGACTGATATGGCTGTTAATTGGGCTGGAGGATTACATCAT
GCTAAGAAATACGAAGCATCAGGATTCTGTTACGTTAATGATATTGTGCTTGCCATCCTTGAATTACTAA
AGTATCATCAGAGAGTCTTATATATTGATATAGATATTCATCATGGTGATGGTGTTGAAGAAGCTTTTTA
TACAACAGATCGTGTAATGACGGTATCATTCCATAAATATGGGGAATACTTTCCTGGCACAGGAGACTTG
AGGGATATTGGTGCTGGAAAAGGCAAATACTATGCTGTCAATTTTCCAATGTGTGATGGTATAGATGATG
AGTCATATGGGGCAGATATTTAAGCCTATTATCTCAAAGGTGATGGAGATGTTCAACCTAGTGCTGTGGT
ATTACAGTGTGGTGCAGACTCATTATCTGGTGATAGACTGGGTTGTTTCAATCTAACAGTCAAAGGTCAT
GCTAAATGTGTAGAAGTTGTAAAAACTTTTAACTTACCATTACTGATGCTTGGAGGAGGTGGCTACACAA
TCCGTAATGTTGCTCGATGTTGGACATATGAGACTGCAGTTGCCCTTGATTGTGAGATTCCCAATGAGTT
GCCATATAATGATTACTTTGAGTATTTTGGACCAGACTTCAAACTGCATATTAGTCCTTCAAACATGACA
AACCAGAACACTCCAGAATATATGGAAAAGATAAAACAGCGTTTGTTTGAAAATTTGCGCATGTTACCTC
ATGCACCTGGTGTCCAGATGCAAGCTATTCCAGAAGATGCTGTTCATGAAGACAGTGGAGATGAAGATGG
AGAAGATCCAGACAAGAGAATTTCTATTCGAGCATCAGACAAGCGGATAGCTTGTGATGAAGAATTCTCA
GATTCTGAGGATGAAGGAGAAGGAGGTCGAAGAAATGTGGCTGATCATAAGAAAGGAGCAAAGAAAGCTA
GAATTGAAGAAGATAAGAAAGAAACAGAGGACAAAAAAACAGACGTTAAGGAAGAAGATAAATCCAAGGA
CAACAGTGGTGAAAAAACAGATACCAAGGACCAAATCAGAAACAGCTCAGCAAACCCCTGAATTTGACAG
TCTCACCAATTTCAGAAATCATTAAAAGAAAAATATTGAAAGGAAAAATGTTTTCTTTTTGAAGACTTCT
GGCTTCATTTTATACTACTTTGGCATGGACTGTATTTATTTTCAAATGGGACTTTTTCGTTTTTGTTTTT
CTGGGCAAGTTTTATTGTGAGATTTTCTTTTATGAAGCAAAAATTTCTTTTCTCCACCATGCTTTATGTG
ATAGTATTTAAAATTGATGTGAGTTATTATGTCAAAAAAACTGATCTATTAAAGAAGTAATTGGCCTTTC
TGAGCTGAAAAAAAAAAAAAAAAAG
HDAC3
SEQ ID NO:96
>gi | 13128861 | ref | NM_003883.2 | Homo sapiens histone deacetylase 3 (HDAC3),
mRNA
GCGGCGGCCGCGGGCGGCGGGCGGCGGAGGTGCGGGGCCTGCTCCCGCCGGCACCATGGCCAAGACCGTG
GCCTATTTCTACGACCCCGACGTGGGCAACTTCCACTACGGAGCTGGACACCCTATGAAGCCCCATCGCC
TGGCATTGACCCATAGCCTGGTCCTGCATTACGGTCTCTATAAGAAGATGATCGTCTTCAAGCCATACCA
GGCCTCCCAGCATGACATGTGCCGCTTCCACTCCGAGGACTACATTGACTTCCTGCAGAGAGTCAGCCCC
ACCAATATGCAAGGCTTCACCAAGAGTCTTAATGCCTTCAACGTAGGCGATGACTGCCCAGTGTTTCCCG
GGCTCTTTGAGTTCTGCTCGCGTTACACAGGCGCATCTCTGCAAGGAGCAACCCAGCTGAACAACAAGAT
CTGTGATATTGCCATTAACTGGGCTGGTGGTCTGCACCATGCCAAGAAGTTTGAGGCCTCTGGCTTCTGC
TATGTCAACGACATTGTGATTGGCATCCTGGAGCTGCTCAAGTACCACCCTCGGGTGCTCTACATTGAGA
TTGACATCCACCATGGTGACGGGGTTCAAGAAGCTTTCTACCTCACTGACCGGGTCATGACGGTGTCCTT
CCACAAATACGGAAATTACTTCTTCCCTGGCACAGGTGACATGTATGAAGTCGGGGCAGAGAGTGGCCGC
TACTACTGTCTGAACGTGCCCCTGCGGGATGGCATTGATGACCAGAGTTACAAGCACCTTTTCCAGCCGG
TTATCAACCAGGTAGTGGACTTCTACCAACCCACGTGCATTGTGCTCCAGTGTGGAGCTGACTCTCTGGG
CTGTGATCGATTGGGCTGCTTTAACCTCAGCATCCGAGGGCATGGGGAATGCGTTGAATATGTCAAGAGC
TTCAATATCCCTCTACTCGTGCTGGGTGGTGGTGGTTATACTGTCCGAAATGTTGCCCGCTGCTGGACAT
ATGAGACATCGCTGCTGGTAGAAGAGGCCATTAGTGAGGAGCTTCCCTATAGTGAATACTTCGAGTACTT
TGCCCCAGACTTCACACTTCATCCAGATGTCAGCACCCGCATCGAGAATCAGAACTCACGCCAGTATCTG
GACCAGATCCGCCAGACAATCTTTGAAAACCTGAAGATGCTGAACCATGCACCTAGTGTCCAGATTCATG
ACGTGCCTGCAGACCTCCTGACCTATGACAGGACTGATGAGGCTGATGCAGAGGAGAGGGGTCCTGAGGA
GAACTATAGCAGGCCAGAGGCACCCAATGAGTTCTATGATGGAGACCATGACAATGACAAGGAAGCAGAT
GTGGAGATTTAAGAGTGGCTTGGGATGCTGTGTCCCAAGGAATTTCTTTTCACCTCTTGGTTGGGCTGGA
GGGAAAAGGAGTGGCTCCTAGAGTCCTGGGGGTCACCCCAGGGCTTTTGCTGACTCTGGGAAAGAGTCTG
GAGACCACATTTGGTTCTCGAACCATCTACCTGCTTTTCCTCTCTCTCCCAAGGCCTGACAATGGTACCT
ATTAGGGATGGAGATACAGACAAGGATAGCTATCTGGGACATTATTGGCAGTGGGCCCTGGAGGCCAGTC
CCTAGCCCCCCCTTGCCCCTTATTTCTTCCCTGCTTCCCTCGAACCCAGAGATTTTTGAGGGATGAACGG
TAGACAGGACTGAGATGCCTCTGACTTCCTCCTCCCCTGATTAAACTTCCTCCCCTTGCTTTTAACGCCC
AGGGAAGATGAAGAGAGAGAGATTTGGAAGGGGCTCTGGCTCCCTAACACCTGAATCCCAGATGATGGGA
AGTATGTTTTCAAGTGTGGGGAGGTATGAAAATGTTCTGTTCTCACTTTTGGCTTTATGTCCATTTTTAC
CACTGTTTTTATCCAATAAACTAAGTCGGTATTTTTTGTACCTTTAAAAAAAAAAAAAAAAAAA
HDAC4
SEQ ID NO:97
>gi | 13259519 | ref | NM_006037.2 | Homo sapiens histone deacetylase 4 (HDAC4),
mRNA
GGAGGTTGTGGGGCCGCCGCCGCGGAGCACCGTCCCCGCCGCCGCCCGAGCCCGAGCCCGAGCCCGCGCA
CCCGCCCGCGCCGCCGCCGCCGCCGCCCGAACAGCCTCCCAGCCTGGGCCCCCGGCGGCGCCGTGGCCGC
GTCCCGGCTGTCGCCGCCCGAGCCCGAGCCCGCGCGCCGGCGGGTGGCGGCGCAGGCTGAGGAGATGCGG
CGCGGAGCGCCGGAGCAGGGCTAGAGCCGGCCGCCGCCGCCCGCCGCGGTAAGCGCAGCCCCGGCCCGGC
GCCCGCGGGCCATTGTCCGCCGCCCGCCCCGCGCCCCGCGCAGCCTGCAGGCCTTGGAGCCCGCGGCAGG
TGGACGCCGCCGGTCCACACCCGCCCCGCGCGCGGCCGTGGGAGGCGGGGGCCAGCGCTGGCCGCGCGCC
GTGGGACCCGCCGGTCCCCAGGGCCGCCCGGCCCCTTCTGGACCTTTCCACCCGCGCCGCGAGGCGGCTT
GCCGGCCCTGCGGGAGCAGCAGCTGCAGCAGGAGCTCCTGGCGCTCAAGCAGAAGCAGCAGATCCAGAGG
CAGATCCTCATCGCTGAGTTCCAGAGGCAGCACGAGCAGCTCTCCCGGCAGCACGAGGCGCAGCTCCACG
AGCACATCAAGCAACAACAGGAGATGCTGGCCATGAAGCACCAGCAGGAGCTGCTGGAACACCAGCGGAA
GCTGGAGAGGCACCGCCAGGAGCAGGAGCTGGAGAAGCAGCACCGGGAGCAGAAGCTGCAGCAGCTCAAG
AACAAGGAGAAGGGCAAAGAGAGTGCCGTGGCCAGCACAGAAGTGAAGATGAAGTTACAAGAATTTGTCC
TCAATAAAAAGAAGGCGCTGGCCCACCGGAATCTGAACCACTGCATTTCCAGCGACCCTCGCTACTGGTA
CGGGAAAACGCAGCACAGTTCCCTTGACCAGAGTTCTCCACCCCAGAGCGGAGTGTCGACCTCCTATAAC
CACCCGGTCCTGGGAATGTACGACGCCAAAGATGACTTCCCTCTTAGGAAAACAGCTTCTGAACCGAATC
TGAAATTACGGTCCAGGCTAAAGCAGAAAGTGGCCGAAAGACGGAGCAGCCCCCTGTTACGCAGGAAAGA
CGGGCCAGTGGTCACTGCTCTAAAAAAGCGTCCGTTGGATGTCACAGACTCCGCGTGCAGCAGCGCCCCA
GGCTCCGGACCCAGCTCACCCAACAACAGCTCCGGGAGCGTCAGCGCGGAGAACGGTATCGCGCCCGCCG
TCCCCAGCATCCCGGCGGAGACGAGTTTGGCGCACAGACTTGTGGCACGAGAAGGCTCGGCCGCTCCACT
TCCCCTCTACACATCGCCATCCTTGCCCAACATCACGCTGGGCCTGCCTGCCACCGGCCCCTCTGCGGGC
ACGGCGGGCCAGCAGGACACCGAGAGACTCACCCTTCCCGCCCTCCAGCAGAGGCTCTCCCTTTTCCCCG
GCACCCACCTCACTCCCTACCTGAGCACCTCGCCCTTGGAGCGGGACGGAGGGGCAGCGCACAGCCCTCT
TCTGCAGCACATGGTCTTACTGGAGCAGCCACCGGCACAAGCACCCCTCGTCACAGGCCTGGGAGCACTG
CCCCTCCACGCACAGTCCTTGGTTGGTGCAGACCGGGTGTCCCCCTCCATCCACAAGCTGCGGCAGCACC
GCCCACTGGGGCGGACCCAGTCGGCCCCGCTGCCCCAGAACGCCCAGGCTCTGCAGCACCTGGTCATCCA
GCAGCAGCATCAGCAGTTTCTGGAGAAACACAAGCAGCAGTTCCAGCAGCAGCAACTGCAGATGAACAAG
ATCATCCCCTAGCCAAGCGAGCCAGCCCGGCAGCCGGAGAGCCACCCGGAGGAGACGGAGGAGGAGCTCC
GTGAGCACCAGGCTCTGCTGGACGAGCCCTACCTGGACCGGCTGCCGGGGCAGAAGGAGGCGCACGCACA
GGCCGGCGTGCAGGTGAAGCAGGAGCCCATTGAGAGCGATGAGGAAGAGGCAGAGCCCCCACGGGAGGTG
GAGCCGGGCCAGCGCCAGCCCAGTGAGCAGGAGCTGCTCTTCAGACAGCAAGCCCTCCTGCTGGAGCAGC
AGCGGATCCACCAGCTGAGGAACTACCAGGCGTCCATGGAGGCCGCCGGCATCCCCGTGTCCTTCGGCGG
CCACAGGCCTCTGTCCCGGGCGCAGTCCTCACCCGCGTCTGCCACCTTCCCCGTGTGTGTGCAGGAGCCC
CCCACCAAGCCGAGGTTCACGACAGGCCTCGTGTATGACACGCTGATGCTGAAGCACCAGTGCACCTGCG
GGAGTAGCAGCAGCCACCCCGAGCACGCCGGGAGGATCCAGAGCATCTGGTCCCGCCTGCAGGAGACGGG
CCTCCGGGGCAAATGCGAGTGCATCCGCGGACGCAAGGCCACCCTGGAGGAGCTACAGACGGTGCACTCG
GAAGCCCACACCCTCCTGTATGGCACGAACCCCCTCAACCGGCAGAAACTGGACAGTAAGAAACTTCTAG
GCTCGCTCGCCTCCGTGTTCGTCCGGCTCCCTTGCGGTGGTGTTGGGGTGGACAGTGACACCATATGGAA
CGAGGTGCACTCGGCGGGGGCAGCCCGCCTGGCTGTGGGCTGCGTGGTAGAGCTGGTCTTCAAGGTGGCC
ACAGGGGAGCTGAAGAATGGCTTTGCTGTGGTCCGCCCCCCTGGACACCATGCGGAGGAGAGCACGCCCA
TGGGCTTTTGCTACTTCAACTCCGTGGCCGTGGCAGCCAAGCTTCTGCAGCAGAGGTTGAGCGTGAGCAG
GATCCTCATCGTGGACTGGGACGTGCACCATGGAAACGGGACCCAGCAGGCTTTCTACAGCGACCCTAGC
GTCCTGTACATGTCCCTCCACCGCTACGACGATGGGAACTTCTTCCCAGGCAGCGGGGCTCCTGATGAGG
TGGGCACAGGGCCCGGCGTGGGTTTCAACGTCAACATGGCTTTCACCGGCGGCCTGGACCCCCCCATGGG
AGACGCTGAGTACTTGGCGGCCTTCAGAACGGTGGTCATGCCGATCGCCAGCGAGTTTGCCCCGGATGTG
GTGCTGGTGTCATCAGGCTTCGATGCCGTGGAGGGCCACCCCACCCCTCTTGGGGGCTACAACCTCTCCG
CCAGATGCTTCGGGTACCTGACGAAGCAGCTGATGGGCCTGGCTGGCGGCCGGATTGTCCTGGCCCTCGA
GGGAGGCCACGACCTGACCGCCATTTGCGACGCCTCGGAAGCATGTGTTTCTGCCTTGCTGGGAAACGAG
CTTGATCCTCTCCCAGAAAAGGTTTTACAGCAAAGACCCAATGCAAACGCTGTCCGTTCCATGGAGAAAG
TCATGGAGATCCACAGCAAGTACTGGCGCTGCCTGCAGCGCACAACCTCCACAGCGGGGCGTTCTCTGAT
CGAGGCTCAGACTTGCGAGAACGAAGAAGCCGAGACGGTCACCGCCATGGCCTCGCTGTCCGTGGGCGTG
AAGCCCGCCGAAAAGAGACCAGATGAGGAGCCCATGGAAGAGGAGCCGCCCCTGTAGCACTCCCTCGAAG
CTGCTGTTCTCTTGTCTGTCTGTCTCTGTCTTGAAGCTCAGCCAAGAAACTTTCCCGTGTCACGCCTGCG
TCCCACCGTGGGGCTCTCTTGGAGCACCCAGGGACACCCAGCGTGCAACAGCCACGGGAAGCCTTTCTGC
CGCCCAGGCCCACAGGTCTCGAGACGCACATGCACGCCTGGGCGTGGCAGCCTCACAGGGAACACGGGAC
AGACGCCGGCGACGCGCAGACACACGGACACGCGGAAGCCAAGCACACTCTGGCGGGTCCCGCAAGGGAC
GCCGTGGAAGAAAGGAGCCTGTGGCAACAGGCGGCCGAGCTGCCGAATTCAGTTGACACGAGGCACAGAA
AACAAATATCAAAGATCTAATAATACAAAACAAACTTGATTAAAACTGGTGCTTAAAGTTTATTACCCAC
AACTCCACAGTCTCTGTGTAAACCACTCGACTCATCTTGTAGCTTATTTTTTTTTTAAAGAGGACGTTTT
CTACGGCTGTGGCCCGCCTCTGTGAACCATAGCGGTGTGCGGCGGGGGGTCTGCACCCGGGTGGGGGACA
GAGGGACCTTTAAAGAAAACAAAACTGGACAGAAACAGGAATGTGAGCTGGGGGAGCTGGCTTGAGTTTC
TCAAAAGCCATCGGAAGATGCGAGTTTGTGCCTTTTTTTTTATTGCTCTGGTGGATTTTTGTGGCTGGGT
TTTCTGAAGTCTGAGGAACAATGCCTTAAGAAAAAACAAACAGCAGGAATCGGTGGGACAGTTICCTGTG
GCCAGCCGAGCCTGGCAGTGCTGGCACCGCGAGCTGGCCTGACGCCTCAAGCACGGGCACCAGCCGTCAT
CTCCGGGGCCAGGGGCTGCAGCCCGGCGGTCCCTGTTTTGCTTTATTGCTGTTTAAGAAAAATGGAGGTA
GTTCCAAAAAAGTGGCAAATCCCGTTGGAGGTTTTGAAGTCCAACAAATTTTAAACGAATCCAAAGTGTT
CTCACACGTCACATACGATTGAGCATCTCCATCTGGTCGTGAAGCATGTGGTAGGCACACTTGCAGTGTT
ACGATCGGAATGCTTTTTATTAAAAGCAAGTAGCATGAAGTATTGCTTAAATTTTAGGTATAAATAAATA
TATATATGTATAATATATATTCCAATGTATTCCAAGCTAAGAAACTTACTTGATTCTTATGAAATCTTGA
TAAAATATTTATAATGCATTTATAGAAAAAGTATATATATATATATAAAATGAATGCAGATTGCGAAGGT
CCCTGCAAATGGATGGCTTGTGAATTTGCTCTCAAGGTGCTTATGGAAAGGGATCCTGATTGATTGAAAT
TCATGTTTTCTCAAGCTCCAGATTGGCTAGATTTCAGATCGCCAACACATTCGCCACTGGGCAACTACCC
TACAAGTTTGTACTTTCATTTTAATTATTTTCTAACAGAACCGCTCCCGTCTCCAAGCCTTCATGCACAT
ATGTACCATGAGTTTTTATAGCAATGAATATAAATTTGCTGTTGATTTTTGTATTGAAATTTTTTCACAA
AAAGATCCTGATAAGCATTGTTTTATGAATTTTACATTTTTCCTCACCATTATAGCAATTTTCTGAATGG
TAATAATGTCTAAATCTTTTTCCTTTCTGAATTCTTGCTTGTACATTTTTTTTTACCTTTCAAAGGTTTT
TAATTATTTTTGTTTTTATTTTTGTACGATGAGTTTTCTGCAGCGTACAGAATTGTTGCTGTCAGATTCT
ATTTCAGAAAGTGAGAGGAGGGACCGTAGGTCTTTTCGGAGTGACACCAACGATTGTGTCTTTTCCTGGT
CTGTCCTAGGAGCTGTATAAAGAAGCCCAGGGGCTCTTTTTAACTTTCAACACTAGTAGTATTACGAGGG
GTGGTGTGTTTTTCCCCTCCGTGGCAkGGGCAGGGAGGGTTGCTTAGGATGCCCGGCCACCCTGGGAGGC
TTGCCAGATGCCGGGGGCAGTCAGCATTAATGAAACTCATGTTTAAACTTCTCTGACCACATCGTCAGGA
TAGAATTCTAACTTGAGTTTTCCAAAGACCTTTTGAGCATGTCAGCAATGCATGGGGCACACGTGGGGCT
CTTTACCCACTTGGGTTTTTCCACTGCAGCCACGTGGCCAGCCCTGGATTTTGGAGCCTGTGGCTGCAAG
GAACCCAGGGACCCTTGTTGCCTGGTGAACCTGCAGGGAGGGTATGATTGCCTGACCAGGACAGCCAGTC
TTTACTCTTTTTCTCTTCAACAGTAACTGACAGTCACGTTTTACTGGTAACTTATTTTCCAGCACATGAA
GCCACCAGTTTCATTCCAAAGTGTATATTGGGTTCAGACTTGGGGGCAGAAGTTCAGACACACCGTGCTC
AGGAGGGACCCAGAGCCGAGTTTCGGAGTTTGGTAAAGTTTACAGGGTAGCTTCTGAAATTAACTCAAAC
TTTTGACCAAATGAGTGCAGATTCTTGGATTCACTTGGTCACTGGGCTGCTGATGGTCAGCTCTGAGACA
GTGGTTTGAGAGCAGGCAGAACGGTCTTGGGACTTGTTTGACTTTCCCCTCCCTGGTGGCCACTCTTTGC
TCTGAAGCCCAGATTGGCAAGAGGAGCTGGTCCATTCCCCATTCATGGCACAGAGCAGTGGCAGGGCCCA
GCTAGCAGGCTCTTCTGGCCTCCTTGGCCTCATTCTCTGCATAGCCCTGTGGGGATCCTGCCACCTGCCC
TCTTACCCCGCCGTGGCTTATGGGGAGGAATGCATCATCTCACTTTTTTTTTTTAAGCAGATGATGGGAT
AACATGGACTGCTCAGTGGCCAGGTTATCAGTGGGGGGACTTAATTCTAATCTCATTCAAATGGAGACGC
CCTCTGCAAAGGCCTGCCAGGGGGAGGCACGTTTCATCTGTCAGCTCACTCCAGCTTCACAAATGTGCTG
AGAGCATTACTGTGTAGCCTTTTCTTTGAAGACACACTCGGCTCTTCTCCACAGCAAGCGTCCAGGGCAG
ATGGCACGAGGATCTGCCCGGCGTCTGCAGGCGGGACCACGTCAGGGAGGGTTCCTTCATGTGTTCTCCC
TGTGGGTCCTTGGACCTTTAGCCTTTTTCTTCCTTTGCAAAGGCCTTGGGGGCACTGGCTGGGAGTCAGC
AAGCGAGCACTTTATATCCCTTTGAGGGAAACCCTGATGACGCCACTGGGCCTCTTGGCGTCTGCCCTGC
CCTCGCGGCTTCCCGCCGTGCCGCAGCGTGCCCACGTGCCCACGCCCCACCAGCAGGCGGCTGTCCCGGA
GGCCGTGGCCCGCTGGGACTGGCCGCCCCTCCCCAGCGTCCCAGGGCTCTGGTTCTGGAGGGCCACTTTG
TCAAGGTGTTTCAGTTTTTCTTTACTTCTTTTGAAAATCTGTTTGCAAGGGGAAGGACCATTTCGTAATG
GTCTGACACAAAAGCAAGTTTGATTTTTGCAGCACTAGCAATGGACTTTGTTGTTTTTCTTTTTGATCAG
AACATTCCTTCTTTACTGGTCACAGCCACGTGCTCATTCCATTCTTCTTTTTGTAGACTTTGGGCCCACG
TGTTTTATGGGCATTATACATATATAAAATATATAGATATAAATATATATGAATATATTTTTTTAAGTTT
CCTACACCTGGAGGTTGCATGGACTGTACGACCGGCATGACTTTATATTGTATACAGATTTTGCACGCCA
AACTCGGCAGCTTTGGGGAAGAAGAAAAATGCCTTTCTGTTCCCCTCTCATGACATTTGCAGATACAAAA
GATGGAAATTTTTCTGTAAAACAAAACCTTGAAGGAGAGGAGGGCGGGGAAGTTTGCGTCTTATTGAACT
TATTCTTAAGAAATTGTACTTTTTATTGTAAGAAAAATAAAAAGGACTACTTAAACATTTGTCATATTAA
GAAAAAAAGTTTATCTAGCACTTGTGACATACCAATAATAGAGTTTATTGTATTTATGTGGAAACAGTGT
TTTAGGGAAACTACTCAGAATTCACAGTGAACTGCCTGTCTCTCTCGAGTTGATTTGGAGGAATTTTGTT
TTGTTTTGTTTTGTTTGTTTCCTTTTATCTCCTTCCACGGGCCAGGCGAGCGCCGCCCGCCCTCACTGGC
CTTGTGACGGTTTATTCTGATTGAGAACTGGGCGGACTCGAAAGAGTCCCCTTTTCCGCACAGCTGTGTT
GACTTTTTAATTACTTTTAGGTGATGTATGGCTAAGATTTCACTTTAAGCAGTCGTGATCTGTGCGAGCA
CTGTGGTTTACAATTATACTTTGCATCGAAAGGAAACCATTTCTTCATTGTAACGAAGCTGAGCGTGTTC
TTAGCTCGGCCTCACTTTGTCTCTGGCATTGATTAAAAGTCTGCTATTGAAAGAAAAAG
HDAC5
SEQ ID NO:98
>gi | 13259520 | ref | NM_005474.2 | Homo sapiens histone deacetylase 5 (HDAC5),
mRNA
ATGAACTCTCCCAACGAGTCGGATGGGATGTCAGGTCGGGAACCATCCTTGGAAATCCTGCCGCGGACTT
CTCTGCACAGCATCCCTGTGACAGTGGAGGTGAAGCCGGTGCTGCCAAGAGCCATGCCCTGTTCCATGGG
GGGTGGGGGTGGAGGCAGCCCCAGCCCTGTGGAGCTACGGGGGGCTCTGGTGGGCTCTGTGGACCCCACA
CTGCGGGAGCAGCAACTGCAGCAGGAGCTCCTGGCGCTCAAGCAGCAGCAGCAGCTGCAGAAGCAGCTCC
TGTTCGCTGAGTTCCAGAAACAGCATGACCACCTGACAAGGCAGCATGAGGTCCAGCTGCAGAAGCACCT
CAAGCAGCAGCAGGAGATGCTGGCAGCCAAGCAGCAGCAGGAGATGCTGGCAGCCAAGCGGCAGCAGGAG
CTGGAGCAGCAGCGGCAGCGGGAGCAGCAGCGGCAGGAAGAGCTGGAGAACCAGCGGCTGGAGCAGCAGC
TGCTCATCCTGCGGAACAAGGAGAAGAGCAAAGAGAGTGCCATTGCCAGCACTGAGGTAAAGCTGAGGCT
CCAGGAATTCCTCTTGTCGAAGTCAAAGGAGCCCACACCAGGCGGCCTCAACCATTCCCTCCCACAGCAC
TAGCGCTGCAGGCCACGGTCACTGTCACCAACTCACACCTCACT3CCTCCCCGAAGCTGTCGACACAGCA
GGAGGCCGAGAGGCAGGCCCTCCAGTCCCTGCGGCAGGGTGGCACGCTGACCGGCAAGTTCATGAGCACA
TCCTCTATTCCTGGCTGCCTGCTGGGCGTGGCACTGGAGGGCGACGGGAGCCCCCACGGGCATGCCTCCC
TGCTGCAGCATGTGCTGTTGCTGGAGCAGGCCCGGCAGCAGAGCACCCTCATTGCTGTGCCACTCCACGG
GCAGTCCCCACTAGTGACGGGTGAACGTGTGGCCACCAGCATGCGGACGGTAGGCAAGCTCCCGCGGCAT
CGGCCCCTGAGCCGCACTCAGTCCTCACCGCTGCCGCAGAGTCCCCAGGCCCTGCAGCAGCTGGTCATGC
AACAACAGCACCAGCAGTTCCTGGAGAAGCAGAAGCAGCAGCAGCTACAGCTGGGCAAGATCCTCACCAA
GACAGGGGAGCTGCCCAGGCAGCCCACCACCCACCCTGAGGAGACAGAGGAGGAGCTGACGGAGCAGCAG
GAGGTCTTGCTGGGGGAGGGAGCCCTGACCATGCCCCGGGAGGGCTCCACAGAGAGTGAGAGCACACAGG
AAGACCTGGAGGAGGAGGACGAGGAAGAGGATGGGGAGGAGGAGGAGGATTGCATCCAGGTTAAGGACGA
GGAGGGCGAGAGTGGTGCTGAAGAGGGGCCCGACTTGGAGGAGCCTGGTGCTGGATACAAAAAACTGTTC
TCAGATGCCCACCCGCTGCAGCCTTTGCAGGTGTACCAGGCGCCCCTCAGCCTGGCCACTGTGCCCCACC
AGGCCCTGGGCCGTGACCCAGTCCTCCCCTGCTGCCCCGGGGGCATGAAGAGCCCCCCAGACCAGCCCGT
CAAGCACCTCTTCACCACAGGTGTGGTCTACGACACGTTCATGCTAAAGCACCAGTGCATGTGCGGGAAC
ACACACGTGCACCCTGAGCATGCTGGCCGGATCCAGAGCATCTGGTCCCGGCTGCAGGAGACAGGCCTGC
TTAGCAAGTGCGAGCGGATCCGAGGTCGCAAAGCCACGCTAGATGAGATCCAGACAGTGCACTCTGAATA
CCACACCCTGCTCTATGGGACCAGTCCCCTCAACCGGCAGAAGCTAGACAGCAAGAAGTTGCTCGGCCCC
ATCAGCCAGAAGATGTATGCTGTGCTGCCTTGTGGGGGCATCGGGGTGGACAGTGACACCGTGTGGAATG
AGATGCACTCCTCCAGTGCTGTGCGCATGGCAGTGGGCTGCCTGCTGGAGCTGGCCTTCAAGGTGGCTGC
AGGAGAGCTCAAGAATGGATTTGCCATCATCCGGCCCCCAGGACACCACGCCGACGAATCCACAGCCATG
GGATTCTGCTTCTTCAACTCTGTAGCCATCACCGCAAAACTCCTACAGCAGAAGTTGAACGTGGGCAAGG
TCCTCATCGTGGACTGGGACATTCACCATGGCAATGGCACCCAGCAGGCGTTCTATAATGACCCCTCTGT
GCTCTACATCTCTCTGCATCGCTATGACAACGGGAACTTCTTTCCAGGCTCTGGGGCTCCTGAAGAGGTT
GGTGGAGGACCAGGCGTGGGGTACAATGTGAACGTGGCATGGACAGGAGGTGTGGACCCCCCCATTGGAG
ACGTGGAGTACCTTACAGCCTTCAGGACAGTGGTGATGCCCATTGCCCACGAGTTCTCACCTGATGTGGT
GCCCTGTGACGCCCCGGCCCCCATCCCTCTGGGCTTCACCATTGTGATTTTGTTTATTTTTTCTATTAAA
AACAAAAAGTCACACATTC
HDAC6
SEQ ID NO:99
>gi | 13128863 | ref | NM_006044.2 | Homo sapiens histone deacetylase 6 (HDAC6),
mRNA
GGGCAGTCCCCTGAGGAGCGGGGCTGGTTGAAACGCTAGGGGCGGGATCTGGCGGAGTGGAAGAACCGCG
GCAGGGGCCAAGCCTCCTCAACTATGACCTCAACCGGCCAGGATTCCACAACAACCAGGCAGCGAAGAAG
TAGGCAGAACCCCCAGTCGCCCCCTCAGGACTCCAGTGTCACACGAAGCAAAAATATTAAAAAGGGAGCC
GTTCCCCGCTCTATCCCCAATCTAGCGGAGGTAAAGAAGAAAGGCAAAATGAAGAAGCTCGGCCAAGCAA
TGGAAGAAGACCTAATCGTGGGACTGCAAGGGATGGATCTGACCTTGAGGCTGAAAGCACTGGCTGGCAC
TGGCTTGGTGTTGGATGAGCAGTTAAATGAATTCCATTGCCTGGGGATCAACAGTTTCCCGGAAGGCCCT
GAGCGGCTCCATGCCATCAAGGAGCAACTGATCCAGGAGGGCCTCCTAAATCGCTGCGTGTCCTTTCAGG
CCCGGTTTGCTGAAAAGGAAGAGCTGATGTTGGTTCACAGCCTAGATATATTGATCTGAATGGAAACAAC
ATTGCTGCTTTCCTGCACGTCCTGCTGCCAGTCGCCCTCGAGTTCCAGCCTCAGCTGGTCCTGGTGGCTG
CTGGATTTGATGCCCTGCAAGGGGACCCCAAGGGTGAGATGGCCGCCACTCCGGCAGGGTTCGCCCAGCT
AACCCACCTGCTCATGGGTCTGGCAGGAGGCAAGCTGATCCTGTCTCTGGAGGGTGGCTACAACCTCCGC
GCCCTGGCTGAAGGCGTCAGTGCTTCGCTCCACACCCTTCTGGGAGACCCTTGCCCCATGCTGGAGTCAC
CTGGTGCCCCCTGCCGGAGTGCCCAGGCTTCAGTTTCCTGTGCTCTGGAAGCCCTTGAGCCCTTCTGGGA
GGTTCTTGTGAGATCAACTGAGACCGTGGAGAGGGACAACATGGAGGAGGACAATGTAGAGGAGAGCGAG
GAGGAAGGACCCTGGGAGCCCCCTGTGCTCCCAATCCTGACATGGCCAGTGCTACAGTCTCGCACAGGGC
TGGTCTATGACCAAAATATGATGAATCACTGCAACTTGTGGGACAGCCACCACCCTGAGGTACCCCAGCG
CATCTTGCGGATCATGTGCCGTCTGGAGGAGCTGGGCCTTGCCGGGCGCTGCCTCACCCTGACACCGCGC
CCTGCCACAGAGGCTGAGCTGCTCACCTGTCACAGTGCTGAGTACGTGGGTCATCTCCGGGCCACAGAGA
AAATGAAAACCCGGGAGCTGCACCGTGAGAGTTCCAACTTTGACTCCATCTATATCTGCCCCAGTACCTT
CGCCTGTGCACAGCTTGCCACTGGCGCTGCCTGCCGCCTGGTGGAGGCTGTGCTCTCAGGAGAGGTTCTG
AATGGTGCTGCTGTGGTGCGTCCCCCAGGACACCACGCAGAGCAGGATGCAGCTTGCGGTTTTTGCTTTT
TCAACTCTGTGGCTGTGGCTGCTCGCCATGCCCAGACTATCAGTGGGCATGCCCTACGGATCCTGATTGT
GGATTGGGATGTCCACCACGGTAATGGAACTCAGCACATGTTTGAGGATGACCCCAGTGTGCTATATGTG
TCCCTGCACCGCTATGATCATGGCACCTTCTTCCCCATGGGGGATGAGGGTGCCAGCAGCCAGATCGGCC
GGGCTGCGGGCACAGGCTTCACCGTCAACGTGGCATGGAACGGGCCCCGCATGGGTGATGCTGACTACCT
AGCTGCCTGGCATCGCCTGGTGCTTCCCATTGCCTACGAGTTTAACCCAGAACTGGTGCTGGTCTCAGCT
GGCTTTGATGCTGCACGGGGGGATCCGCTGGGGGGCTGCCAGGTGTCACCTGAGGGTTATGCCCACCTCA
CCCACCTGCTGATGGGCCTTGCCAGTGGCCGCATTATCCTTATCCTAGAGGGTGGCTATAACCTGACATC
CATCTCAGAGTCCATGGCTGCCTGCACTCGCTCCCTCCTTGGAGACCCACCACCCCTGCTGACCCTGCCA
CGGCCCCCACTATCAGGGGCCCTGGCCTCAATCACTGAGACCATCCAAGTCCATCGCAGATACTGGCGCA
GCTTACGGGTCATGAAGGTAGAAGACAGAGAAGGACCCTCCAGTTCTAAGTTGGTCACCAAGAAGGCACC
CCAACCAGCCAAACCTAGGTTAGCTGAGCGGATGACCACACGAGAAAAGAAGGTTCTGGAAGCAGGCATG
GGGGAAGTCACCTCGGCATCATTTGGGGAAGAGTCCACTCCAGGCCAGACTAACTCAGAGACAGCTGTGG
TGGCCCTCACTCAGGACCAGCCCTCAGAGGCAGCCACAGGGGGAGCCACTCTGGCCCAGACCATTTCTGA
GCCAGCCATTGGGGGAGCCATGCTGGGCCAGACCACCTCAGAGGAGGCTGTCGGGGGAGCCACTCCGGAC
CAGACCACCTCAGAGGAGACTGTGGGAGGAGCCATTCTGGACCAGACCACCTCAGAGGATGCTGTTGGGG
GAGCCACGCTGGGCCAGACTACCTCAGAGGAGGCTGTAGGAGGAGCTACACTGGCCCAGACCACCTCGGA
GGCAGCCATGGAGGGAGCCACACTGGACCAGACTACGTCAGAGGAGGCTCCAGGGGGCACCGAGCTGATC
CAAACTCGTCTAGCCTCGAGCACAGACCACCAGACCCCCCCAACCTCACCTGTGCAGGGAACTACACCCC
AGATATCTCCCAGTACACTGATTGGGAGTCTCAGGACCTTGGAGCTAGGCAGCGAATCTCAGGGGGCCTC
AGAATCTCAGGCCCCAGGAGAGGAGAACCTACTAGGAGAGGCAGCTGGAGGTCAGGACATGGCTGATTCG
ATGCTGATGCAGGGATCTAGGGGCCTCACTGATCAGGCCATATTTTATGCTGTGACACCACTGCCCTGGT
GTCCCCATTTGGTGGCAGTATGCCCCATACCTGCAGCAGGCCTAGACGTGACCCAACCTTGTGGGGACTG
TGGAACAATCCAAGAGAATTGGGTGTGTCTCTCTTGCTATCAGGTCTACTGTGGTCGTTACATCAATGGC
CACATGCTCCAACACCATGGAAATTCTGGACACCCGCTGGTCCTCAGCTACATCGACCTGTCAGCCTGGT
GTTACTACTGTCAGGCCTATGTCCACCACCAGGCTCTCCTAGATGTGAAGAACATCGCCCACCAGAACAA
GTTTGGGGAGGATATGCCCCACCCACACTAAGCCCCAGAATACGGTCCCTCTTCACCTTCTGAGGCCCAC
GATAGACCAGCTGTAGCTCATTCCAGCCTGTACCTTGGATGAGCGGTAGCCTCCCACTGCATCCCATCCT
GAATATCCTTTGCAACTCCCCAAGAGTGCTTATTTAAGTGTTAATACTTTTAAGAGAACTGCGACGATTA
ATTGTGGATCTCCCCCTGCCCATTGCCTGCTTGAGGGGCACCACTACTCCAGCCCAGAAGGAAAGGGGGG
CAGCTCGTGGCCCCAAGAGGGAGCTGATATCATGAGGATAACATTGGCGGGAGGGGAGTTTAACTGGCAG
GCATGGCAAGGTTGCATATGTAATAAAGTACAAGCTGTT
HDAC7
SEQ ID NO:100
>gi | 13259521 | ref | NM_015401.1 | Homo sapiens histone deacetylase 7A (HDAC7A),
transcript variant 1, mRNA
ATAATACCTACCTTGCAGGACCACGACAGGATTAAGTGAGGAAAAACCCCCATGAGAGTGTTTTGCCATT
GTCAAGTGAGCCTGAGGGAGGCTGAGGGGGGATCAGGCTGTATCATGCCCCCGAGGACAAACTTTCCAGT
TTACCCTGCTCCCTCTCTCTGTCCCTAGGCTGCCCCAGGCCCTGTGCAGACACACCAGGCCCTCAGCCGC
AGCCCATGGACCTGCGGGTGGGCCAGCGGCCCCCAGTGGAGCCCCCACCAGAGCCCACATTGCTGGCCCT
GCAGCGTCCCCAGCGCCTGCACCACCACCTCTTCCTAGCAGGCCTGCAGCAGCAGCGCTCGGTGGAGCCC
ATGAGGCTCTCCATGGACACGCCGATGCCCGAGTTGCAGGTGGGACCCCAGGAACAAGAGCTGCGGCAGC
TTCTCCACAAGGACAAGAGCAAGCGAAGTGCTGTAGCCAGCAGCGTGGTCAAGCAGAAGCTAGCGGAGGT
GATTCTGAAAAAACAGCAGGCGGCCCTAGAAAGAACAGTCCATCCCAACAGCCCCGGCATTCCCTACAGA
ACCCTGGAGCCCCTGGAGACGGAAGGAGCCACCCGCTCCATGCTCAGCAGCTTTTTGCCTCCTGTTCCCA
GCCTGCCCAGTGACCCCCCAGAGCACTTCCCTCTGCGCAAGACAGTCTCTGAGCCCAACCTGAAGCTGCG
CTATAAGCCCAAGAAGTCCCTGGAGCGGAGGAAGAATCCACTGCTCCGAAAGGAGAGTGCGCCCCCCAGC
CTCCGGCGGCGGCCCGCAGAGACCCTCGGAGACTCCTCCCCAAGTAGTAGCAGCACGCCCGCATCAGGGT
GCAGCTCCCCCAATGACAGCGAGCACGGCCCCAATCCCATCCTGGGCTCGGAGGCGCTCTTGGGCCAGCG
GCTGCGGCTGCAGGAGACTTCTGTGGCCCCGTTCGCCTTGCCGACAGTGTCCTTGCTGCCCGCAATCACT
CTGGGGCTGCCCGCCCCTGCCAGGGCTGACAGTGACCGCAGGACCCATCCGACTCTGGGCCCTCGGGGGC
CAATCCTGGGGAGCCCCCACACTCCCCTCTTCCTGCCCCATGGCTTGGAGCCCGAGGCTGGGGGCACCTT
GCCCTCTCGCCTGCAGCCCATTCTCCTCCTGGACCCCTCAGGCTCTCATGCCCCGCTGCTGACTGTGCCC
GGGCTTGGGCCCTTGCCCTTCCACTTTGCCCAGTCCTTAATGACCACCGAGCGGCTCTCTGGGTCAGGCC
TCCACTGGCCACTGAGCCGGACTCGCTCAGAGCCCCTGCCCCCCAGTGCCACCGCTCCCCCACCGCCGGG
CCCCATGCAGCCCCGCCTGGAGCAGCTCAAAACTCACGTCCAGGTGATCAAGAGGTCAGCCAAGCCGAGT
GAGAAGCCCCGGCTGCGGCAGATACCCTCGGCTGAAGACCTGGAGACAGATGGCGGGGGACCGGGCCAGG
TGGTGGCGATGGCCTGGAGCACAGGGAGCTGGGCCATGGGCAGCCTGAGGCCAGAGGCCCCCGCTCCTCT
CCAGCAGCACCCTCAGGTGTTGCTCTGGGACAGCAGCGACTGGCTGGGCGGCTCCCCCGGGGAAGAACCC
GGGGACACTGTGCTGCTTCCTCTGGCCCAGGGTGGGCACCGGCCTCTGTCCCGGGCTCAGTCTTCCCCAG
CCGCACCTGCCTCACTGTCAGCCCCAGAGCCTGCCAGCCAGGCCCGAGTCCTCTCCAGCTCAGAGACCCC
TGCCAGGACCCTGCCCTTCACCACAGGGCTGATCTATGACTCGGTCATGCTGAAGCACCAGTGCTCCTGC
GGTGACAACAGCAGGCACCCGGAGCACGCCGGCCGCATCCAGAGCATCTGGTCCCGGCTGAAGGAGCGGG
GGCTCCGGAGCCAGTGTGAGTGTCTCCGAGGCCGGAAGGCCTCCCTGGAAGAGCTGCAGTCGGTCCACTC
TGAGCGGCACGTGCTCCTCTACGGCACCAACCCGCTCAGCCGCCTCAAACTGGACAACGGGAAGCTGGCA
GGGCTCCTGGCACAGCGGATGTTTGTGATGCTGCCCTGTGGTGGGGTTGGGGTGGACACTGACACCATCT
GGAATGAGCTTCATTCCTCCAATGCAGCCCGCTGGGCCGCTGGCAGTGTCACTGACCTCGCCTTCAAAGT
GGCTTCTCGTGAGCTAAGAATGGTTTCGCTGTGGTGCGGCCCCCAAGGACGACCATGCAGATCATTCAAA
GCCATGGGCTTCTGCTCTTCAACTCAGTGGCCATCGCCTGCCGGCAGCTGCAACAGCAGAGCAAGGCCAA
GCAAGATCCTCATTGTAGACTGGGACGTGCACCATGGCAACGGCACCCAGCAAACCTTCTACCAAGACCC
CAGTGTGCTCTACATCTCCCTGCATCGCCATGACGACGGCAACTTCTTCCCGGGGAGTGGGGCTGTGGAT
GAGGTAGGGGCTGGCAGCGGTGAGGGCTTCAATGTCAATGTGGCCTGGGCTGGAGGTCTGGACCCCCCCA
GGGGGATCCTGAGTACCTGGCGCTTTCAGGATAGTCGTGAGCCCATCGCCCGAGAGTTCTCTCAAGATGA
CCTAGTCCTGGTGTCTGCTGGATTTGATGCTGGTGAGGGTCACCCGGCCCCACTGGGTGGCTACCATGTT
TCTGCCAAATGTTTTGGATACATGACGCAGCAACTGATGAACCTGGCAGGAGGCGCAGTGGTGAAGGCCT
TGGAGGGTGGCCATGACCTCACAGCCATCTGTGACGCCTCTGAGGCCTGTGTGGCTGCTCTTCTGGGTAA
CAGGGTGGATCCCCTTTCAGAAGAAGGCTGGAAACAGAAACCCAACCTCAATGCCATCCGCTCTAAAGGT
GCCGTGATCCGGGTGCACAGTAAATACTGGGGCTGCATGCAGCGACCTGGCCTCCTGTCCAGACTCCTAG
TGCCTAGAGTGCCAGGGGCTGACAAAGAAGAAGTGGAGGCAGTGACCGCACTGGCGTCCCTCTCTGTGGG
CATCCTGGCTGAAGATAGGCCCTCGGAGCAGCTGGTGGAGGAGGAAGAACCTATGAATCTCTAAGGCTCT
GGACCATCTGCCCGCCCACCATGCCCTTGGACCTGTTCTCTTCTACCCCTGGCAATAGCCCCAAGCTGCG
CCTGGGTCTTTAGAGATCCTGTGGCAAGTAGTTGGAACCAGAGAACAGCCTGCCTGCTTTGAAAGGTTAT
CCCAGGGAGCGTGAGAAAATCCCTGGGTCTAGAATGGGAACTGGAGAGGACCCTGAGAGGAGACGGGCTG
GGCGGCGACCCCCACAGGGCTCTCGAGAACAGATTCTCCCCTCCAGTATGGGCCCTGGCTGTGGCCCCAA
TTCCTCAGGACTGCACAGAGGAGGACTGGCTCCGGCTCCGTCGGGCTCACCCTTAACCACTATTCCAGTC
TCTGCAAACCCCAGACTTTGCACACAGCCCCAGGCTCCACACAGAAATGTGAACTTGGCCTCAGACAGGC
TGGCCCTTCCTAGGCTCTAGGGGCTAGGGGGGAGTGGGGAGCCAAGAGGTCCCATATTCCTGAGTGCAGG
GGTAGTCCCTCTCACCTGCTTCCTCAGACGACTAGGGAAGCTTCCCTCTACCACCGGGCACTGAGAGGCA
GCTCCCTGACAGCCGAGACTGGCAGCCCTCCATCTGGTCCGTACCCTCGCCAGAGGCCCCCCTACATCAA
CCTCCTGGCGATGCCCTGGTGGAGCAGATGGGTGCTCTGGGAGTCCTGTGCTTCCTGATCCAATGGTGCC
AAACCCTTCATCTCCCCCAGAAGCGCAGCATACCCCTGGGACCCCTCGGCCACTGCCCACTCGGGGAGCC
TTCTCTGTTTCTGGGGCCTCCCCCACCATAGCTCTGATTCCCACCCCACATAGGAATAGCCTGACTGAGG
GGGAAGGGGTGGGAGAGAAGATACAGACATGGAGGAGGGGAGGCTGCTCTGGCAAAGTCTTCAAGGCTTT
TGGGGGTCCAGGCCTGGGGTCAAGAAGGAAAATGTGTGTGAGCATGTGTGTGAGTGAGGCGTGTGTGTGA
GCGTGTGTGTGAGTGAGGCGTGTGTGTGTGTCTTTCCTAGGACCCACCATACCCTGTGTATGTATGCATG
TTTTTGTAAAAAGGAAGAAAATGGAAAAAAATCTGAACAATAAATGTTTTATTTGCTTTAAAAAAAAAAA
AAAAAA
>gi | 13259523 | ref | NM_016596.2 | Homo sapiens histone deacetylase 7A (HDAC7A),
transcript variant 2, mRNA SEQ ID NO:101
ATAATACCTACCTTGCAGGACCACGACAGGATTAAGTGAGGAAAAACCCCCATGAGAGTGTTTTGCCATT
GCTCCCTGACAGCCGAGACTGGCAGCCCTCCATCTGGTCCGTACCCTCGCCAGAGGCCCCCCTACATCAA
TTACCCTGCTCCCTCTCTCTGTCCCTAGGCTGCCCCAGGCCCTGTGCAGACACACCAGGCCCTCAGCCGC
AGCCCATGGACCTGCGGGTGGGCCAGCGGCCCCCAGTGAGCCCCCACCAGAGCCCAACTGGCCAGTCGGA
GCAGCGTCCCCAGCGCCTGCACCACCACCTCTTCCTAGCAGGCCTGCAGCAGCAGCGCTCGGTGGAGCCC
ATGAGGCTCTCCATGGACACGCCGATGCCCGAGTTGCAGGTGGGACCCCAGGAAAGAGAGCTGCGGCCGC
TTCTCCACAAGGACAAGAGCAAGCGAAGTGCTGTAGCCAGCAGCGTGGTCAAGCAGAAGCTAGACGAGGT
GATTCTGAAAAAACAGCAGGCGGCCCTAGAAACAGTCCATCCCAACAGCCCCGGCATTCCCTACGACAGA
ACCCTGGAGCCCCTGGAGACGGAAGGAGCCACCCGCTCCATGCTCAGCAGCTTTTTGCCTCCTGTTCCCA
GCCTGCCCAGTGACCCCCCAGAGCACTTCCCTCTGCGCAAGACAGTCTCTGAGCCCAACCTGAAGCTGCG
CTATAAGCCCAAGAAGTCCCTGGAGCGGAGGAAGAATCCACTGCTCCGAAAGGAGAGTGCGCCCCCCAGC
CTCCGGCGGCGGCCCGCAGAGACCCTCGGAGACTCCTCCCCAAGTAGTAGCAGCACGCCCGCATCAGGGT
GCAGCTCCCCCAATGACAGCGAGCACGGCCCCAATGGGATCCTGGCGACAGTGACCGCAGGACCACATCC
GACTCTGGGCCCTCGGGGGGCCAATCCTGGGGAGCCCCCACACTCCCCTCTTCCTGCCCATGGCTTGGAG
CCCGAGGCTGGGGGCACCTTGCCCTCTCGGCTGAGCCCATTCTCCTCCTGGACCCCCTCAGGCTCTCATG
CCCCGCTGCTGACTGTGCCCGGGCTTGGGCCCTTGCCCTTCCACTTTGCCCAGTCCTTAATGACCACCGA
GCGGCTCTCTGGGTCAGGCCTCCACTGGCCACTGAGCCGGACTCGCTCAGAGCCCCTGCCCCCCAGTGCC
ACCGCTCCCCCACCGCCGGGCCCCATGCAGCCCCGCCTGGGCAGCTCAAAACTCACGTCCAGGTGAATCA
AGAGGTCAGCCAAGCCGAGTGAGAAGCCCCGGCTGCGGCAGATACCCTCGGCTGAAGACCTGGAGACAGA
TGGCGGGGGACCGGGCCAGGTGGTGGACGATGGCCTGGAGCACAGGGAGCTGGGCCATGGGCAGCCTGAG
GCCAGAGGCCCCGCTCCTCTCCAGCAGCACCCTCAGGTGTTGCTCTGGGAACAGCAGCGACTGGCTGGGC
GGCTCCCCCGGGGCAGCACCGGGGACACTGTGCTGCTTCCTCTGGCCCAGGGTGGGCACCGGCCTCTGTC
CCGGGCTCAGTCTTCCCCAGCCGCACCTGCCTCACTGTCAGCCCCAGAGCCTGCCAGCCAGGCCCGAGTC
CTCTCCAGCTCAGAGACCCCTGCCAGGACCCTGCCCTTCACCACAGGGCTGATCTATGACTCGGTCATGC
TGAAGCACCAGTGCTCCTGCGGTGACAACAGCAGGCACCCGGAGCACGCCGGCCGCATCCAGAGCATCTG
GTCCCGGCTGCAGGAGCGGGGGCTCCGGAGCCAGTGTGAGTGTCTCCGAGGCCGGAAGGCCTCCCTGGAA
GAGCTGCAGTCGGTCCACTCTGAGCGGCACGTGCTCCTCTACGGCACCAACCCGCTCAGCCGCCTCAAAC
TGGAGGTCTGGACCCCCCCATGGGGGATCCTGAGTACCTGGCTGATTTCAGGATAGTCGTGAATCCCATC
GTCCCGGCTGCAGGAGCGGGGGCTCCGGAGCCAGTGTGAGTGTCTCCGAGGCCGGAAGGCCTCCCTGGAA
GAGCTGCAGTCGGTCCACTCTGAGCGGCACGTGCTCCTCTACGGCACCAACCCGCTCAGCCGCCTCAAAC
TGGACAACGGGAAGCTGGCAGGGCTCCTGGCACAGCGGATGTTTGTGATGCTGCCCTGTGGTGGGGTTGG
GGTGGACACTGACACCATCTGGAATGAGCTTCATTCCTCCAATGCAGCCCGCTGGGCCGCTGGCAGTGTC
ACTGACCTCGCCTTCAAAGTGGCTTCTCGTGAGCTAAAGAATGGTTTCGCTGTGGTGCGGCCCCCAGGAC
ACCATGCAGATCATTCAACAGCCATGGGCTTCTGCTTCTTCAACTCAGTGGCCATCGCCTGCCGGCAGCT
GCAACAGCAGAGCAAGGCCAGCAAGATCCTCATTGTAGACTGGGACGTGCACCATGGCAACGGCACCCAG
CAAACCTTCTACCAAGACCCCAGTGTGCTCTACATCTCCCTGCATCGCCATGACGACGGCAACTTCTTCC
CGGGGAGTGGGGCTGTGGATGAGGTAGGGGCTGGCAGCGGTGAGGGCTTCAATGTCAATGTGGCCTGGGC
TGGAGGTCTGGACCCCCCCATGGGGGATCCTGAGTACCTGGCTGCTTTCAGGATAGTCGTGATGCCCATC
GCCCGAGAGTTCTCTCCAGACCTAGTCCTGGTGTCTGCTGGATTTGATGCTGCTGAGGGTCACCCGGCCC
CACTGGGTGGCTACCATGTTTCTGCCAAATGTTTTGGATACATGACGCAGCAACTGATGAACCTGGCAGG
AGGCGCAGTGGTGCTGGCCTTGGAGGGTGGCCATGACCTCACAGCCATCTGTGACGCCTCTGAGGCCTGT
GTGGACTGCTCTTCTGGGTAACAAATGGATCCCCTTTCAGAAGAAGGCTGGAAACAGAAACCCAACCTCA
ATGCCATCCGCTCTCTGGAGGCCGTGATCCGGGTGCACAGTAAATACTGGGGCTGCATGAAGCGCCTGGC
CTCCTGTCCAGACTCCTGGGTGCCTAGAGTGCCAGGGGCTGACAAAGAAGAAGTGGAGGCAGTGACCGCA
CTGGCGTCCCTCTCTGTGGGCATCCTGGCTGAAGATAGGCCCTCGGAGCAGCTGGTGGAGGAGGAAGAAC
CTATGAATCTCTAAGGCTCTGGAACCATCTGCCCGCCCACCATGCCCTTGGGACCTGGTTCTCTTCTAAC
CCCTGGCAATAGCCCCCATTCCTGGGTCTTTAGAGATCCTGTGGGCAAGTAGTTGGAACCAGAGAACAGC
CTGCCTGCTTTGACAGTTATCCCAGGGAGCGTGAGAAAATCCCTGGGTCTAGAATGGGAACTGGAGAGGA
CCCTGAGAGGAGACGGGCTGGGCGGCGACCCCCACAGGGCTCTCGAGAACAGATTCTCCCCTCCAGTATG
GGCCCTGGCTGTGGCCCCCATTCCTCAGGACTGCACAGAGGAGGACTGGCTCCGGCTCCGTCGGGCTCAC
CCTTAACCACTATTCCTGGCTCTGCAAACCCCAGACTTTGCACACAGCCCCAGGCTCCACACAGAAATGT
GAACTTGGCCTCAGACAGGCTGGCCCTTCCTAGGCTCTAGGGGCTAGGGGGGAGTGGGGAGCCAAGAGGT
CCCATATTCCTGAGTGCAGGGGTAGTCCCTCTCACCTGCTTCCTCAGACGACTCTGGAAGCTTCCCTCTA
CCACCGGGCACTGAGACGAAGCTCCCTGACAGCCGAGACTGGCAGCCCTCCATCTGGTCCGTACCCTCGC
CAGAGGCCCCCCTACATCAACCTCCTGGCGATGCCCTGGTGGAGCAGATGGGTGCTCTGGGAGTCCTGTG
CTTCCTGATCCAATGGTGCCAAACCCTTCATCTCCCCCAGAAGCGCAGCATACCCCTGGGACCCCTCGGC
CACTGCCCACTCGGGGAGCCTTCTCTGTTTCTGGGGCCTCCCCCACCATAGCTCTGATTCCCACCCCACA
TAGGAATAGCCTGACTGAGGGGGAAGGGGTGGGAGAGAAGATACAGACATGGAGGAGGGGAGGCTGCTCT
GGCAAGTCTTCAAGGCTTTTGGGGGTCCAGGCCTGGGGTCAAGAAGGAAAATGTGTGTGAGCATGTGTGT
TGAGTGAGGCGTGTGTGTGAGCGTGTGTGTGAGTGAGGCGTGTGTGTGTGTCTTTCCTAGGACCCACCAT
ACCCTGTGTATGTATGCATGTTTTTGTAAAAAGGAAGAAAATGGAAAAAATCTGAACAATAAATGTTATT
ATTTGCTTTAAAAAAAAAAAAAAAAA
>gi | 7662279 | ref | NM_014707.1 | Homo sapiens histone deacetylase 9 (HDAC9-
PENDING), transcript variant 3, mRNA SEQ ID NO: 102
GGGGAAGAGAGGCACAGACACAGATAGGAGAAGGGCACCGGCTGGAGCCACTTGCAGGACTGAGGGTTTT
TGCAACAAAACCCTAGCAGCCTGAAGAACTCTAAGCCAGATGGGGTGGACGAGAGCAGAGGCCTCTTGGC
TCAGCAAAGAATGCACAGTATGATCAGCTCAGTGGATGTGAAGTCAGAAGTTCCTGTGGGCCTGGAGCCC
ATCTCACCTTTAGACCTAAGGACAGACCTCAGGATGATGATGCCCGTGGTGGACCCTGTTGTCCTGAAGA
AGCAATTGCAGCAGGAATTACTTCTTATCCAGCAGCAGCAACAAATCCAGAAGCAGCTTCTGATAGCAGA
GTTTCAGAAACAGCATGAGAACTTGACACGGCAGCACCAGGCTCAGCTTCAGGAGCATATCAAGGAACTT
CTAGCCATAAAACAGCAACAAGAACTCCTAGAAAAGGAGCAGAAACTGGAGCAGCAGAGGCAAGAACAGG
AAGTAGAGAGGCATCGCAGAGAACAGCAGCTTCCCTCCTCTCAGAGGCAAGATAGAGGACGAGAAAGGGC
AGTGGCAAGTACAGAAGTAAAGCAGAAGCTTCAAGAGTTCCTACTGAGTAAATCAGCAACGAAAGACACT
CCAACTAATGGAAAAAATCATTCCGTGAGCCGCCATCCCAAGCTCTGGTACACGGCTGCCCACCACACAT
CATTGGATCAAAGCTCTCCACCCCTTAGTGGAACATCTCCATCCTAACAGTACACATTACCAGGAGCACA
AGATGCAAAGGATGATTTCCCCCTTCGAAAAACTGCCTCTGAGCCCAACTTGAAGGTGCGGTCCAGGTTA
AAACAGAAAGTGGCAGAGAGGAGAAGCAGCCCCTTACTCAGGCGGAAGGATGGAAATGTTGTCACTTCAT
TCAAGAAGCGAATGTTTGAGGTGACAGAATCCTCAGTCAGTAGCAGTTCTCCAGCGCTCTGGTCCCAGTT
ACCAAACAATGGGCCAACTGGAAGTGTTACTGAAAAATGAGACTTCGGTTTTGCCCCCTACCCCTCATGC
GAGCAAATGGTTTCACAGCAACGCATTCTAATTCATGAAGATTCCATGAACCTGCTAAGTCTTTATACCT
CTCCTTCTTTGCCCAACATTACCTTGGGGCTTCCCGCAGTGCCATCCCAGCTCAATGCTTCGAATTCACT
CAAAGAAAAGCAGAAGTGTGAGACGCAGACGCTTAGGCAAGGTGTTCCTCTGCCTGGGCAGTATGGAGGC
AGCATCCCGGCATCTTCCAGCCACCCTCATGTTACTTTAGAGGGAAAGCCACCCAACAGCAGCCACCAGG
CTCTCCTGCAGCATTTATTATTGAAAGAACAAATGCGACAGCAAAAGCTTCTTGTAGCTGGTGGAGTTCC
CTTACATCCTCAGTCTCCCTTGGCAACAAAAGAGAGAATTTCACCTGGCATTAGAGGTACCCACAAATTG
CCCCGTCACAGACCCCTGAACCGAACCCAGTCTGCACCTTTGCCTCAGAGCACGTTGGCTCAGCTGGTCA
TTCAACAGCAACACCAGCAATTCTTGGAGAAGCAGAAGCAATACCAGCAGCAGATCCACATGAACAAACT
GCTTTCGAAATCTATTGAACAACTGAAGCAACCAGGCAGTCACCTTGAGGAAGCAGAGGAAGAGCTTCAG
GGGGACCAGGCGATGCAGGAAGACAGAGCGCCCTCTAGTGGCAACAGCACTAGGAGCGACAGCAGTGCTT
GTGTGGATGACACACTGGGACAAGTTGGGGCTGTGAAGGTCAAGGGGAACCAGTGGACAGTGATGAAGAA
TGCTCAGATCCAGGAAATGGAATCTGGGGAGCAGGCTGCTTTTATGCAACAGGTAATAGGCAAAGATTTA
GCTCCAGGATTTGTAATTAAAGTCATTATCTGAACATGAAATGCATTGCAGGTTTGGTAAATGGATATGA
TTTCCTATCAGTTTATATTTCTCTATGATTTGAGTTCAGTGTTTAAGGATTCTACCTAATGCAGATATAT
GTATATATCTATATAGAGGTCTTTCTATATACTGATCTCTATATAGATATCAATGTTTCATTGAAAATCC
ACTGGTAAGGAAATACCTGTTATACTAAAATTATGATACATAATATCTGAGCAGTTAATAGGCTTTAAAT
TTATCCCAAAGCCTGCTACACCAATTACTTCTAAAGAAAACAAATTCACTGTTATTTTGAGTTTATGTGT
TGAGATCAGTGACTGCTGGATAGTCTCCCAGTCTGATCAATGAAGCATTCGATTAGTTTTTGATTTTTTG
CAACATCTAGAATTTAATTTTCACATCACTGTACATAATGTATCATACTATAGTCTTGAACACTGTTAAA
GGTAGTCTGCCCCTTCCTTCCTCTCTCTTTTTTTAGTTAAGTAGAAATGTTCTGGTCACCATGCCAGTAG
TCCTAGGTTATTGTGTAGGTTGCAATTGAACATATTAGGAATACAGGTGGTTTTAAATATATAGATGCAA
ATTGCAGCACTACTTTAAATATTAGATTATGTCTCACATAGCACTGCTCATTTTACTTTTATTTTGTGTA
ATTTGATGACACTGTCTATCAAAAAAGAGCAAATGAAGCAGATGCAAATGTTAGTGAGAAGTAATGTGCA
GCATTATGGTCCAATCAGATACAATATTGTGTCTACAATTGCAAAAAACACAGTAACAGGATGAATATTA
TCTGATATCAAGTCAAAATCAGTTTGAAAAGAAGGTGTATCATATTTTATATTGTCACTAGAATCTCTTA
AGTATAATTCCATAATGACATGGGCATATACCGTAACATTCTGGCAAATAACAATTAGAAAAGATAGGTT
TAACAAAAAAATTTACTTGTATATAATGCACCTTCAGGAGGACTATGTCCTTTGATGCTATAAAATACAA
ACAACTTTGAAGGCAACAGAAGACACTGTTTATTCAAGTCAGTTCTTTGTCAGGTTCCTGCTGTTCTCCT
ACAGAAAAGTGATTCTGTGAGGGTGAACAGGAAATGCCTTGTGGAAACAGGAAGTCCAAGTGATTCATGT
ACTGAGGAATGTAGGAAAAAAATCTGAGGATAGTGCTTTACTCTTTCTGTTTTTTAAAGGGCACTCTATG
AATTGATTTATTGTCTAAGAAAATAACACCACAAGTAGGGAAATTGTTACGGAAGCTTTTCACTGGAACA
TTTCCTTCATATTCCCTTTTGATATGTTTACCTTGTTTTATAGGTTTACTTTTGTTAAGCTAGTTAAAGG
TTCGTTGTATTAAGACCCCTTTAATATGGATAATCCAAATTGACCTAGAATCTTTGTGAGGTTTTTTCTA
TTAAAATATTTATATTTCTAAATCCGAGGTATTTCAAGGTGTAGTATCCTATTTCAAAGGAGATATAGCA
GTTTTGCCAAATGTAGACATTGTTCACTGTATGTTATTGGCACGTGTTGTTTACATTTTGCAGTGAAATT
TTAAAAAATATTTCTTTAAAAATGTTACTGCTAAAGATACATTATCCTTTTTTAAAAAGTCTCCATTCAA
TTAAATTAACATAACTAGAAGTTAGAAAGTTTAAAAGTTTTCCACATAATGAAAGTCCTTCTGATAATTT
GACAAATAGCTATAATAGGAACACTCCCTATCACCAACATATTTTGGTTAGACGTATATTCCTTTTCCAT
ATGACTTTTTGTCAGTTGTTTTGCATTAAAAATATGGCATGCCTAAGATAAAATTGTATATTTTTACCAT
CTCATAAATATTCATTTTCTTCAAAGTCTTTTTTTCAATCTCATAAAAAAGGGATAGTGCATCTTTTAAA
TACATTTTATTTGGGGAGGAACATGTGGCTGAGCAGACTTTTGTATAATATTACTTCAAAGATATGTAAT
CACAAACAAAAAAAACTATTTTTTATAATGTCATTTGAGAGAGTTTCATCAGTACAGTTGGTGGACGTTA
ATTGTTTGAATTTGATAGTCTTTGAATTTAATCAAGAAACTACCTGGAACCAGTGAAAAGGAAAGCTTGA
CTTAAATAATCTTAGAATTAATTGATAAATGTCTCTTTTAAAATCTACTGTATTTATTATAATTTACACC
CTTGAAGGTGATCTCTTGTTTTGTGTTGTAAATATATTGTTTGTATGTTTCCCTTCTTGCCTTCTGTTAT
AAGTCTCTTCCTTTCTCAAATAAAGTTTTTTTTAAAAG
HDAC8
SEQ ID NO:103
>gi | 8923768 | ref | NM_018486.1 | Homo sapiens hostone deacetylase 8 (HCAC8), mRNA
GCCAGATCTGGAAGGTGGCTGCGGAACGGTTTTAAGCGGAAGATGGAGGAGCCGGAGGAACCGGCGGACA
GTGGGCAGTCGCTGGTCCCGGTTTATATCTATAGTCCCGAGTATGTCAGTATGTGTGACTCCCTGGCCAA
GATCCCCAAACGGGCCAGTATGGTGCATTCTTTGATTGAAGCATATGCACTGCATAAGCAGATGAGGATA
GTTAAGCCTAAAGTGGCCTCCATGGAGGAGATGGCCACCTTCCACACTGATGCTTATCTGCAGCATCTCC
AGAAGGTCAGCCAAGAGGGCGATGATGATCATCCGGACTCCATAGAATATGGGCTAGGTTATGACTGCCC
AGCCACTGAAGGGATATTTGAGCTATGCAGCAGCTATAGGAGGGGCTACGATCACAGCTGCCCAATGCCT
ATTGACGGAATGTGCAAAGTAGCAATTAACTGGTCTGGAGGGTGGCATCATGCAAAGAAAGATGAAGCAT
CTGGTTTTTGTTATCTCAATGATGCTGTCCTGGGAATATTACGATTTGCGACGGAAATTTGAGCGTATTC
CTACGTGGATTTGGATCTGCACCATGGAGATGGTGTAGAAGACGCATTCAGTTTCACCTCCAAAGTCATG
ACCGTGTCCCTGCACAAATTCTCCCCAGGATTTTTCCCAGGAACAGGTGACGTGTCTGATGTTGGCCTAG
GGAAGGGACGGTACTACAGTGTAAATGTGCCCATTCAGGATGGCATACAAGATGAAAAATATTACCAGAT
CTGTGAAAGTGTACTAAAGGAAGTATACCAAGCCTTTAATCCCAAAGCAGTGGTCTTACAGCTGGGAGCT
GACACAATAGCTGGGGATCCCATGTGCTCCTTTAACATGACTCCAGTGGGAATTGGCAAGTGTCTTAAGT
ACATCCTTCAATGGCAGTTGGCAACACTCATTTTGGGAGGAGGAGGCTATAACCTTGCCAACACGGCTCG
ATGCTGGACATACTTGACCGGGGTCATCCTAGGGAAAACACTATCCTCTGAGATCCCAGATCATGAGTTT
TTCACAGCATATGGTCCTGATTATGTGCTGGAAATCACGCCAAGCTGCCGGCCAGACCGCAATGAGCCCC
ACCGAATCCAACAAATCCTCAACTACATCAAAGGGAATCTGAAGCATGTGGTCTAGTTGACAGAAAGAGA
TCAGGTTTCCAGAGCTGAGGAGTGGTGCCTATAATGAAGACAGCGTGTTTATGCAAGCAGTTTGTGGAAT
TTGTGACTGCAGGGAAAATTTGAAAGAAATTACTTCCTGAAAATTTCCAAGGGGCATCAAGTGGCAGCTG
GCTTCCTGGGGTGAAGAGGCAGGCACCCCAGAGTCCTCAACTGGACCTAGGGGAAGAAGGAGATATCCCA
CATTTAAAGTTCTTATTTAAAAAAACACACACACACAAATGAAATTTTTAATCTTTGAAAATTATTTTTA
AGCGAATTGGGGAGGGGAGTATTTTAATCATCTTAAATGAAACAGATCAGAAGCTGGATGAGAGCAGTCA
CCAGTTTGTAGGGCAGGAGGCAGCTGAGAGGCAGGGTTTGGGCCTCAGGACCATCAAGGTGGAGCCCTGG
GAGAGAGGGTACTGATCAGCAGACTGGGAGGTGGGGAGAAGTCCGCTGGTGTTGTTTTAGTGTTATATAT
CTTTGGTTTTTTTAATAAAATCTTTGAAAACCTAAAAAAAAAAAAAAAAAAAAAAAAAAAAAA
hSIRT2
SEQ ID NO:104
>gi | 13775599 | ref | NM_012237.2 | Homo sapiens sirtuin silent mating type
information regulation 2 homolog 2 (S. cerevisiae) (SIRT2),
transcript variant 1, mRNA
GTGTTGTACGAAAGCGCGTCTGCGGCCGCAATGTCTGCTGAGAGTTGTAGTTCTGTGCCCTATCACGGCC
ACTCCCATTTCTGGTGCCGTCACGGGACAGAGCAGTCGGTGACAGGACAGAGCAGTCGGTGACGGGACAC
AGTGGTTGGTGACGGGACAGAGCGGTCGGTGACAGCCTCAAGGGTTCAGCACCGCGCCCATGGCAAGAGC
CAGACCCCTCTCACCCTCTGGAGACCCAGGCAGGGAAGGTGCAGGAGGCTCAGGACTCAGATTCAGACTC
TGAGGGAGGAGCCGCTGGTGGAGAAGCAGACATGGACTTCCTGCGGAACTTATTCTCCCAGACGCTCAGC
CTGGGCAGCCAGAAGGAGCGTCTGCTGGACGAGCTGACCTTGGAAGGGGTGGCCCGGTACATGCAGAGCG
AACGCTGTCGCAGAGTCATCTGTTTGGTGGGAGCTGGAATCTCCACATCCGCAGCATCCCCGACTTTTCG
CTCTCCATCCACCGGCCTCTATGACAACCTAGAGAAGTACCATCTTCCCTACCCAGAGGCCATCTTTGAG
ATCAGCTATTTCAAGAAACATCCGGAACCCTTCTTCGCCCTCGCCAAGGAACTCTATCCTGGGCAGTTCA
AGCCACCATCTGTCACTACTTCATGCGCCTGCTGAAGGACAAGGGGCTACTCCTGCGCTGCTACACAGCA
GAACATAGATACCCTGGAGCGAATAGCCGGGCTGGAACAGGAGGACTTGGTGGAGGCGCACGGCACCTTC
TACACATCACACTGCGTCAGCGCCAGCTGCCGGCACGAATACCCGCTAAGCTGGATGAAAGAGAAGATCT
TCTCTGAGGTGACGCCCAAGTGTGAAGACTGTCAGAGCCTGGTGAAGCCTGATATCGTCTTTTTTGGTGA
GAGCCTCCCAGCGCGTTTCTTCTCCTGTATGCAGTCAGACTTCCTGAAGGTGGACCTCCTCCTGGTCATG
GGTACCTCCTTGCAGGTGCAGCCCTTTGCCTCCCTCATCAGCAAGGCACCCCTCTCCACCCCTCGCCTGC
TCATCAACAGGAGAAAGCTGGCCAGTCGGACCCTTTCCTGGGGATGATTATGGGCCTCGGAGCCAGGCAT
GGACTTTGACTCCAAGAAGGCCTACAGGGACGTGGCCTGGCTGGGTGAATGCGACCAGGGCTGCCTGGCC
GTTGCTGAGCTCCTTGGATGGAAGAAGGAGCTGGAGGACCTTGTCCGGAGGGAGCACGCCAGCATAGATG
CCCAGTCGGGGGCGGGGGTCCCCAACCCCAGCACTTCAGCTTCCCCCAAAGAAGTCCCCGCCACCTGCTC
GGACGAGGCCAGGACAACAGAGAGGGAGACCCCAGTGACAGCTGCATCTCCCAGGCGGGATGCCCAAGAC
TCCTCGGGACAGCTGAGCCCCAACCGGGCCTGGCCCCCTCTTAACCAGCAGTTCTTGTCTGGGGAGATTC
GGACCCACTTCCCATGCTGGATGGGCAGAAGACATTGCTTATTGGAGACAAATTAAAAACAAAAACAACT
AAC
>gi | 13775601 | ref | NM_030593.1 | Homo sapiens sirtuin silent mating type
information regulation 2 homolog 2 (S. cerevisiae) (SIRT2),
transcript variant 2, mRNA SEQ ID NO:105
CGAAAGCGCGTCTGCGGCCGCAATGTCTGCTGAGAGTTGTAGTTCTGTGCCCTATCACGGCCACTCCCAT
TTCTGGTGCCGTCACGGGACAGAGCAGTCGGTGACAGGACAGAGCAGTCGGTGACGGGACACAGTGGTTG
GTGACGGGACAGAGCGGTCGGTGACAGCCTCAAGGGCTTCAGCACCGCGCCCATGGCAGAGCCAGACCGA
CTCAGATTCAGACTCTGAGGGAGGAGCCGCTGGTGGAGAAGCAGACATGGACTTCCTGCGGAACTTATTC
TCCCAGACGCTCAGCCTGGGCAGCCAGAAGGAGCGTCTGCTGGACGAGCTGACCTTGGAAGGGGTGGCCC
GGTACATGCAGAGCGAACGCTGTCGCAGAGTCATCTGTTTGGTGGGAGCTGGAATCTCCACATCCGCAGG
CATCCCCGACTTTCGCTCTCCATCCACCGGCCTCTATGACAACCTAGAGAAGTACCATCTTCCCTACCCA
GAGGCCATCTTTGAGATCAGCTATTTCAAGAAACATCCGGAACCCTTCTTCGCCCTCGCCAAGGAACTCT
ATCCTGGGCAGTTCAAGCCAACCATCTGTCACTACTTCATGCGCCTGCTGAAGGACAAGGGGCTACTCCT
GCGCTGCTACACGCAGAACATAGATACCCTGGAGCGAATAGCCGGGCTGGAACAGGAGGACTTGGTGGAG
GCGCACGGCACCTTCTACACATCACACTGCGTCAGCGCCAGCTGCCGGCACGAATACCCGCTAAGCTGGA
TGAAAGAGAAGATCTTCTCTGAGGTGACGCCCAAGTGTGAAGACTGTCAGAGCCTGGTGAAGCCTGATAT
CGTCTTTTTTGGTGAGAGCCTCCCAGCGCGTTTCTTCTCCTGTATGCAGTCAGACTTCCTGAAGGTGGAC
CTCCTCCTGGTCATGGGTACCTCCTTGCAGGTGCAGCCCTTTGCCTCCCTCATCAGCAAGGCACCCCTCT
CCACCCCTCGCCTGCTACATCAACAAGGAGAAAGCTGGCCAGTCGGACCCTTTCCTGGGGATGATTATGG
CCTCGGAGGAGGCATGGACTTTGACTCCAAGAAGGCCTACAGGGACGTGGCCTGGCTGGGTGAATGCGAC
CAGGGCTGCCTGGCCCTTGCTGAGCTCCTTGGATGGAAGAAAGGAGCTGGAGGACCTTGTCCGGAGGAGC
ACGCCAGCATAGATGCCCAGTCGGGGGCGGGGGTCCCCAACCCCAGCACTTCAGCTTCCCCCAAGAAGTC
CCCGCCACCTGCCAAGGACGAGGCCAGGACAACAGAGAGGGAGAACCCCAGTGACAGCTGCATCTCACCA
GGCGGGATGCCGAGCTCCTCAGGGACAGCTGAGCCCCAACCGGGCCTGGCCCCCTCTTAACCAGCAGTTC
TTGTCTGGGGAGCTCAGAACATCCCCCCAATCTCTTACAGCTCCCTCCCCAAAACTGGGTCCCAGCAACC
CTGGCCCCCAACCCCAGCAAATCTCTAACACCTCCTAGAGGCCAAGGCTTAAACAGGCATCTCTACCAGC
CCCACTGTCTCTAACCACTCCTGGGCTAAGGAGTAACCTCCCTCATCTCTAACTGCCCCCACGGGGCCAG
GGCTACCCCAGAACTTTTAACTCTTCCAGGACAGGGAGCTTCGGGCCCCCACTCTGTCTCCTGCCCCCGG
GGGCCTGTGGCTAAGTAAACCATACCTAACCTACCCCAGTGTGGGTGTGGGCCTCTGAATATAACCCACA
CCCAGCGTAGGGGGAGTCTGAGCCGGGAGGGCTCCCGAGTCTCTGCCTTCAGCTCCAAAGTGGGTGGTAG
GGCCCCCTTCACGTGGGACCCACTTCCCATGCTGGTGGGCAGAAGACAGGGCTTATTGGAGACAAAATTA
AAAACAAAAACAACTAACAAAAAAAAAAAAAAAAAAAAAA
hSIRT3
SEQ ID NO:106
gi | 13775603 | ref | NM_012239.3 | Homo sapiens sirtuin silent mating type
information regulation 2 homolog 3 (S. cerevisiae) (SIRT3), mRNA
GGCGCCGGGGGCGGGGGTGGGAGGCGGAGGCGGGGCCGGGGCGCCGCGGGCGGGGCGCCGGGGGCGGGGC
GAGTCCGGAGGACTCCTCGGACTGCGCGGAACATGGCGTTCTGGGGTTGGCGCGCCGCGGCAGCCCTCCG
GCTGTGGGGCCGGGTAGTTGAACGGGTCGAGGCCGGGGGAGGCGTGGGGCCGTTTCAGGCCTGCGGTCGT
CGGCTGGTGCTTGGCGGCAGGGACGATGTGAGTGCGGGGCTGAGAGGCAGCCATGGGGCCCGCGGTGAGC
CCTTGGACCCGGCGCGCCCCTTGCAGAGGCCTCCCAGACCCGAGGTGCCCAGGGCATTCCGGAGGCAGCC
GAGGGCAGCAGCTCCCAGTTTCTTCTTTTCGAGTATTAAAGGTGGAAGAAGGTCCATATCTTTTTCTGTG
GGTGCTTCAAGTGTTGTTGGAAGTGGAGGCAGCAGTGACAAGGGGAAGCTTTCCCTGCAGGATGTAGCTG
AGCTGATTCGGGCCAGAGCCTGCCAGAGGGTGGTGGTCATGGTGGGGGCCGGCATCAGCACACCCAGTGG
CATTCCAGACTTCAGATCGCCGGGGAGTGGCCTGTACAGCAACCTCCAGCAGTACGATCTCCCGTACCCC
GAGGCCATTTTTGAACTCCCATTCTTCTTTCACAACCCCAAGCCCTTTTTCACTTTGGCCAAGGAGCTGT
ACCCTGGAAACTACAAGCCCAACGTCACTCACTACTTTCTCCGGCTGCTTCATGACAAGGGGCTGCTTCT
GCGGCTCTACACGCAGAACATCGATGGGCTTGAGAGAGTGTCGGGCATCCCTGCCTCAAAGCTGGTTGAA
GCTCATGGAACCTTTGCCTCTGCCACCTGCACAGTCTGCCAAAGACCTTCCCAGGGGAGGACATTCGGGG
CTGACGTGATGGCAGACAGGGTTCCCCGCTGCCCGGTCTGCACCGGCGTTGTGAAGCCCGACATTGTGTT
CTTTGGGGAGCCGCTGCCCCAGAGGTTCTTGCTGCATGTGGTTGATTTCCCCATGGCAGATCTGCTGCTC
ATCCTTGGGACCTCCCTGGAGGTGGAGCCTTTTGCCAGCTTGACCGAGGCCGTGCGGAGCTCAGTTCCCC
GACTGCTCATCAACCGGGACTTGGTGGGGCCCTTGGCTTGGCATCCTCGCAGCAGGGACGTGGCCCAGCT
GGGGGACGTGGTTCACGGCGTGGAAAGCCTAGTGGAGCTTCTGGGCTGGACAGAAGAGATGCGGGACCTT
GTGCAGCGGGAAACTGGGAAGCTTGATGGACCAGACAAATAGGATGATGGCTGCCCCCACACAATAAATG
GTAACATAGGAGACATCCACATCCCAATTCTGACAAGACCTCATGCCTGAAGACAGCTTGGGCAGGTGAA
ACCAGAATATGTGAACTGAGTGGACACCCGAGGCTGCCACTGGAATGTCTTCTCAGGCCATGAGCTGCAG
TGACTGGTAGGGCTGTGTTTACAGTCAGGGCCACCCCGTCACATATACAAAGGAGCTGCCTGCCTGTTTG
CTGTGTTGAACTCTTCACTCTGCTGAAGCTCCTAATGGAAAAAGCTTTCTTCTGACTGTGACCCTCTTGA
ACTGAATCAGACCAACTGGAATCCCAGACCGAGTCTGCTTTCTGTGCCTAGTTGAACGGCAAGCTCGGCA
TCTGTTGGTTACAAGATCCAGACTTGGGCCGAGCGGTCCCCAGCCCTCTTCATGTTCCGAAGTGTAGTCT
TGAGGCCCTGGTGCCGCACTTCTAGCATGTTGGTCTCCTTTAGTGGGGCTATTTTTAATGAGAGAAAATC
TGTTCTTTCCAGCATGAAATACATTTAGTCTCCTCAAAGGGAAAAAAAAAAAAAAAAAAAAAAAAAAAAA
AAAAAAAAAAAAAAAAAAAAAAAAAAAAAAAAAAAAA
hSIRT4
SEQ ID NO:107
>gi | 6912661 | ref | NM_012240.1 | Homo sapiens sirtuin silent mating type
information regulation 2 homolog 4 (S. cerevisiae) (SIRT4), mRNA
GTCCGTAGAGCTGTGAGAGAATGAAGATGAGCTTTGCGTTGACTTTCAGGTCAGCAAAAGGCCGTTGGAT
CGCAAACCCCAGCCAGCCGTGCTCGAAAGCCTCCATTGGGTTATTTGTGCCAGCAAGTCCTCCTCTGGAC
CCTGAGAAGGTCAAAGAGTTACAGCGCTTCATCACCCTTTCCAAGAGACTCCTTGTGATGACTGGGGCAG
GAATCTCCACCGAATCGGGGATACCAGACTACAGGTCAGAAAAAGTGGGGCTTTATGCCCGCACTGACCG
CAGGCCCATCCAGCATGGTGATTTTGTCCGGAGTGCCCCAATCCGCCAGCGTACTGGGCGAGAAAACTTC
GTAGGCTGGCCTCAATTCTCCTCCCACCAGCCTAACCCTGCACACTGGGCTTTGAGCACCTGGGAGAAAC
TCGGAAAGCTGTACTGGTTGGTGACCCAAAATGTGGATGCTTTGCACACCAAGGCGGGGAGTCGGCGCCT
GACAGAGCTCCACGGATGCATGGACAGGGTCCTGTGCTTGGATTGTGGGGAACAGACTCCCCGGGGGGTG
CTGCAAGAGCGTTTCCAAGTCCTGAACCCCACCTGGAGTGCTGAGGCCCATGGCCTGGCTCCTGATGGTG
ACGTCTTTCTCTCAGAGGAGCAAGTCCGGAGCTTTCAGGTCCCAACCTGCGTTCAATGTGGAGGCCATCT
GAAACCAGATGTCGTTTTCTTCGGGGACACAGTGAACCCTGACAAGGTTATTTTTGTGCACAAGCGTGTA
AAAGAAGCCGACTCCCTCTTGGTGGTGGGATCATCCTTGCAGGTATACTCTGGTTACAGGTTTATCCTCA
CTGCCTGGGAGAAGAAGCTCCCGATGCAATACTGAACATTGGGCCCACACGGTCGGATGACTTGGCGATG
TCTGAAACTGAATTCTCGTTGTGGAGAGTTGCTGCCTTTGATAGACCCATGCTGACCACAGCCTGATATT
CCGAGAAGGTCAAAGAGTTACAGCGCTTCATCACCCTTTCCAAGAGACTCCTTGTGATGACTGGGGCAGA
TCCATTCCCATTCAACAGAGTAGGGTGCACTGACAAAGTATAGAAGGTTCTAGGTATCTTAATGTGATGG
ATATTCTTAATTAAAACTCATTTTTTTAAATAAAAAATTGTTCAGCTTTAAAA
hSIRT5
SEQ ID NO:108
>gi | 13787213 | ref | NM_012241.2 | Homo sapiens sirtuin silent mating type
information regulation 2 homolog 5 (S. cerevisiae) (SIRT5),
transcript variant 1, mRNA
CCGGAGCGCGGTCGGGACACAGCGCCTCTAGGAGAAAGCCTGGAAGGCGCTCCGGGGGTACCCAGAGCTC
TTAGCGGGCCGGCAGCATGTGCGGGGCCCAAGTAAATGGAAATGTTTTCTAACATATAAAAACCTACAGA
AGAAGAAAATAATTTTCTGGATCAAATTAGAAGTCTGTATTATATTGATGTCTCCAGATTCAAATATATT
AGAAAGCAGCCGTGGAGACAACCATCTTCATTTTGGGAGAAATAACTAAAGCCCGCCTCAAGCATTAGAA
CTACAGACAAACCCTGATGCGACCTCTCCAGATTGTCCCAAGTCGATTGATTTCCCAGCTATATTGTGGC
CTGAAGCCTCCAGCGTCCACACGAAACCAGATTTGCCTGAAAATGGCTCGGCCAAGTTCAAGTATGGCAG
ATTTTCGAAAGTTTTTTGCAAAAGCAAAGCACATAGTCCATCTCAGGAGCTGGTGTTAGTGCAGAAAAAG
TGGTGTTCCGACCTTCAGAGGAGCTGGAGGTTATTGGAGAAAATGGCAAGCCCAGGACCTGGCGACTCCC
CTGGCCTTTGCCCACAACCCGTCCCGGGTGTGGGAGTTCTACCACTACCGGCGGGAGGTCATGGGGAGCA
AGGAGCCCAACGCCGGGCACCGCGCCATAGCCGAGTGTGAGACCCGGCTGGGCAAGCAGGGCCGGCGAGT
CGTGGTCATCACCCAGAACATCGATGAGCTGCACCGCAAGGCTGGCACCAAGAACCTTCTGGAGATCCAT
GGTAGCTTATTTAAAACTCGATGTACCTCTTGTGGAGTTGTGGCTGAGAATTACAAGAGTCCAATGGGTC
CAGCTTTATCAGGAAAAGGTGCTCCAGAACCTGGAACTCAAGATGCCAGCATCCCAGTTGAGAAACTTCC
CCGGTGTGAAGAGGCAGGCTGCGGGGGCTTGCTGCGACCTCACGTCGTGTGGTTTGGAGAAAACCTGGAT
CCTGCCATTCTGGAGGAGGTTGACAGAGAGCTCGCCCACTGTGATTTATGTCTAGTGGTGGGCACTTCCT
CTGTGGTGTACCCAGCAGCCATGTTTGCCCCCCAGGTGGCTGCCAGGGGCGTGCCAGTGGCTGAATTTAA
CACGGAGACCACCCCAGCTACGAACAGATTCAGGTTTCATTTCCAGGGACCCTGTGGAACGACTCTTCCT
GAAGCCCTTGCCTGTCATGAAAATGAAACTGTTTCTTAAGTGTCCTGGGGAAGAAAGAAATTACAGTATA
TCTAAGAACTAGGCCACACGCAGAGGAGAAATGGTCTTATGGGTGGTGAGCTGAGTACTGAACAATCTAA
AAATAGCCTCGATTCCCTCGCTGGAATCCAACCTGTTGATAAGTGATGGGGGTTTAGAAGTAGCAAAGAA
GCACCCACATTCAAAAGTCACAGAACTGGAAAGTTAATTCAAGGGGTAAAAACCCTACTATTCTGAAATT
TATCTTTGATGTGTATGGTTGGTTATTGGGAGGGAAAAATTTTGTAAATTAGATTGTCTAAAAAAAATAG
TTATTCTGATTATATTTTTGTTATCTGGGCAAAGTAGAAGTCAAGGGGTAAAAACCCTACTATTCTGATT
TTTGCACAAGTTTTAGTGGAAAATAAAATCACACTCTACAGTAAAAAAAAAAAAAAAAAA
>gi | 13787214 | ref | NM_031244.1 | Homo sapiens sirtuin silent mating type
information regulation 2 homolog 5 (S. cerevisiae) (SIRT5),
transcript variant 2, mRNA SEQ ID NO:109
ATTCGGGGGCGCGAGCTGCCCCAGTAAATGGAAATGTTTTCTAACATATAAAAACCTACAGAAGAAGAAA
ATAATTTTCTGGATCAAATTAGAAGTCTGTATTATATTGATGTCTCCAGATTCAAATATATTAGAAAGCA
GCCGTGGAGACAACCATCTTCATTTTGGGCGAAATAACTAAAGCCCGCCTCAAGCATTAGAACTACAGAC
AAACCCTGATGCGACCTCTCCAGATTGTCCCAAGTCGATTGATTTCCCAGCTATATTGTGGCCTGAAGCC
TCCAGCGTCCACACGAAACCAGATTTGCCTGAAAATGGCTCGGCCAAGTTCAAGTATGGCAGATTTTCGA
AAGTTTTTTGCAAAAGCAAAGCACATAGTCATCATCTCAGGAGCTGGTGTTAGTGCAGAAAGTGGTGTTC
CGACCTTCAGAGGAGCTGGAGGTTATTGGAGAAAATGGCAAGCCCAGGACCTGGCGACTCCCCTGGCCTT
TGCCCACAACCCGTCCCGGGTGTGGGAGTTCTACCACTACCGGCGGGAGGTCATGGGGAGCAAGGAGCCC
AACGCCGGGCACCGCGCCATAGCCGAGTGTGAGACCCGGCTGGGCAAGCAGGGCCGGCGAGTCGTGGTCA
TCACCCAGAACATCGATGAGCTGCACCGCAAGGCTGGCACCAAGAACCTTCTGGAGATCCATGGTAGCTT
ATTTAAAACTCGATGTACCTCTTGTGGAGTTGTGGCTGAGAATTACAAGAGTCCAATTTGTCCAGCTTTA
TCAGGAAAAGGTGCTCCAGAACCTGGAACTCAAGATGCCAGCATCCCAGTTGAGAAACTTCCCCGGTGTG
AAGAGGCAGGCTGCGGGGGCTTGCTGCGACCTCACGTCGTGTGGTTTGGAGAAAACCTGGATCCTGCCAT
TCTGGAGGAGGTTGACAGAGAGCTCGCCCACTGTGATTTATGTCTAGTGGTGGGCACTTCCTCTGTGGTG
TACCCAGCAGCCATGTTTGCCCCCCAGGTGGCTGCGAGGGGCGTGCCAGTGGCTGAATTTAACACGGAGA
CCACCCCCAGCTACGAACAGATTCAGTCATTTGATCTCCATCTCATCTCTAATTATTATAAAGAATTAAA
TAGAAGAGATGCTGGTGATAGTCATCTCATCCTACTCAATTTTTCAAAGGCAGAAACCAACCCTGGAGCA
CCACCCCAGCTACGAACAGATTCAGTCATTTGATCTCCATCTCATCTCTAATTATTATAAAGAATTAAAA
CAAGTCATCATTGTAGAAAAGCAAGAAAATGCAGATAGAGAAAAAGAAGAAAAATAAAACTGGAGTATTC
CTGTAAATAAGGTTTCATTGGAACACAGCTGTGGCCATATGTTTGTATATTGTGTGTGGCTGCTTTTGCA
TTAGGATGACAGAGGTGAATAGTTGCAACAGAGACTGGCTGGTCTGCAAGCCTAAATATATGTCCAATGT
TGCTACTTTTAACCGATTTTCTTGTACCTAAATAGGCAGCTAGATTGCTGCCTATATTTTAAATATCCGG
ATTTGGATCTCACAAGACAACCTTAGGCCTACACAGGTTGTTCAGATTCTTTGCCCCAGTTCTAATTAGT
GACAAAGGCATAGAATTCTCCTCCACAGGAATGTATTTCTATTTTCAAGGTGATTAATTAGTTCCAGTTT
TGGTTTTGTCGTTTCCCCATGTCCGATGCTTATATTGGATGATTTCTGATACCTGACTATTCCAAGCGTA
AACCCTAGGCATTTTTGAATTTAAAAAAAAAAAAAAAAA
hSIRT6
SEQ ID NO:110
>gi | 7706709 | ref | NM_016539.1 | Homo sapiens sirtuin silent mating type
information regulation 2 homolog 6 (S. cerevisiae) (SIRT6), mRNA
GCTTCCGGCGGAAGCGGCCTCAACAAGGGAAACTTTATTGTTCCCGTGGGGCAGTCGAGGATGTCGGTGA
ATTACGCGGCGGGGCTGTCGCCGTACGCGGACAAGGGCAAGTGCGGCCTCCCGGAGATCTTCGACCCCCC
GGAGGAGCTGGAGCGGAAGGTGTGGGAACTGGCGAGGCTGGTCTGGCAGTCTTCCAGTGTGGTGTTCCAC
ACGGGTGCCGGCATCAGCACTGCCTCTGGCATCCCCGACTTCAGGGGTCCCCACGGAGTCTGGACCATGG
AGGAGCGAGGTCTGGCCCCCAAGTTCGACACCACCTTTGAGAGCGCGCGGCCCACGCAGACCCACATGGC
GCTGGTGCAGCTGGAGCGCGTGGGCCTCCTCCGCTTCCTGGTCAGCCAGAACGTGGACGGGCTCCATGTG
CGCTCAGGCTTCCCCAGGGACAAACTGGCAGAGCTCCACGGGAACATGTTTGTGGAAGAATGTGCCAAGT
GTAAGACGCAGTACGTCCGAGACACAGTCGTGGGCACCATGGGCCTTAAGGCCACGGGCGGCTCTGCACC
CGTGGCTAAGGCAAGGGGGCTGCGAGCCTGCAGGGGAGAGCTGAGGGACACCATCCTAGACTGGGAGGAC
TCCCTGCCCGACCGGGACCTGGCACTCGCCGATGAGGCCAGCAGGAACGCCGACCTGTCCATCACGCTGG
GTACATCGCTGCAGATCCGGCCCAGCGGGAACCTGCCGCTGGCTACCAAGCGCCGGGGAGGCCGCCTGGT
CATCGTCAACCTGCAGCCCACCAAGCACGACCGCCATGCTGACCTCCGCATCCATGGCTACGTTGACGAG
GTCATGACCGGCTCATGGAGCACCTGGGGCTGGAGATCCCCGCCTGGGACGGCCCCCGTGTGCTGGAGAA
GGGCGCTGCCACCCCTGCCCCGCCCGCCCACCCCCAAGCTGGAGCCCAAGGAGGAATCTCCCACCCGGAT
CAACGGCTCTATCCCCGCCGGCCCCAAGCAGGAGCCTGCGCCCAGCACAACGGCTCAGAGCCGCCAAAGC
CCCAAACGGGAGCGGCCCACCAGCCCTGCCCCCCACAGACCCCCCAAAAAGGGTGAAGGCCAGGCGGTCC
CCAGCTGACCAGGGTGCTTACTTGTTTCTGTCCCCGGGAGCCTCAGGGCTCTGAGAGCTGTGCCAGGGGT
ACACCTGCCCTCCGTGGTCCCTCCCTGGGCTCCAGGGGCCTCTGGTGCGGTTCCGGGAAGAAGCCACACC
CCAGAGGTGACAGCTGAGCCCCTGCCACACCCCAGCCTCTGACTTGCTGTGTTGTCCAGAGGTGAGGCTG
GGCCCTCCCTGGTCTCCAGCTTAACAGGAGTGAACTCCCTCTGTCCCCAACGGCCTCCCTTCTGGGCCCC
CTACAGCCCACCCTACCCCTCCTCCATGGGCCCTGCAGGAGGGGAGACCCACCTTGAAGTGGGGGATCAG
TAGAGGCTTGCACTGCCTTTGGGGCTGGAGGGAGACGTGGGTCCACCAGGCTTCTGGAAAAGTCCTCAAT
GCAATAAAAACAATTTCTTTCTTGCAAA
hSIRT7
SEQ ID NO:111
>gi | 7706711 | ref | NM_016538.1 | Homo sapiens sirtuin silent mating type
information, regulation 2 homolog 7 (S. cerevisiae) (SIRT7), mRNA
GCGGAAGCGGAAGAGCAGGTCTCCAGGGGAGCGATGGCAGCCGGGGGTCTGAGCCGCTCCGAGCGCAAAG
CGGCGGAGCGGGTCCGGAGGTTGCGGGAGGAGCAGCAGAGGGAGCGCCTCCGCCAGGTGTCGCGCATCCT
GATTAAGGCGGCGGCGGAGCGCAGCGCCGAGGAGGGCCGGCTGCTGGCCGAGAGCGGACCTGGTAACGAC
GCTTCCGGCGGAAGCGGCCTCAACAAGGGAAACTTTATTGTTCCCGTGGGGCAGTCGAGGATGTCGGTGA
ATTACGCGGCGGGGCTGTCGCCGTACGCGGACAAGGGCAAGTGCGGCCTCCCGGAGATCTTCGACCCCCC
GGAGGAGCTGGAGCGGAAGGTGTGGGAACTGGCGAGGCTGGTCTGGCAGTCTTCCAGTGTGGTGTTCCAC
ACGGGTGCCGGCATCAGCACTGCCTCTGGCATCCCCGACTTCAGGGGTCCCCACGGAGTCTGGACCATGG
AGGAGCGAGGTCTGGCCCCCAAGTTCGACACCACCTTTGAGAGCGCGCGGCCCACGCAGACCCACATGGC
GCTGGTGCAGCTGGAGCGCGTGGGCCTCCTCCGCTTCCTGGTCAGCCAGAACGTGGACGGGCTCCATGTG
CGCTCAGGCTTCCCCAGGGACAAACTGGCAGAGCTCCACGGGAACATGTTTGTGGAAGAATGTGCCAAGT
GTAAGACGCAGTACGTCCGAGACACAGTCGTGGGCACCATGGGCCTTAAGGCCACGGGCGGCTCTGCACC
CGTGGCTAAGGCAAGGGGGCTGCGAGCCTGCAGGGGAGAGCTGAGGGACACCATCCTAGACTGGGAGGAC
TCCCTGCCCGACCGGGACCTGGCACTCGCCGATGAGGCCAGCAGGAACGCCGACCTGTCCATCACGCTGG
GTACATCGCTGCAGATCCGGCCCAGCGGGAACCTGCCGCTGGCTACCAAGCGCCGGGGAGGCCGCCTGGT
CATCGTCAACCTGCAGCCCACCAAGCACGACCGCCATGCTGACCTCCGCATCCATGGCTACGTTGACGAG
GTCATGACCGGCTCATGGAGCACCTGGGGCTGGAGATCCCCGCCTGGGACGGCCCCCGTGTGCTGGAGAA
GGGCGCTGCCACCCCTGCCCCGCCCGCCCACCCCCAAGCTGGAGCCCAAGGAGGAATCTCCCACCCGGAT
ACGGGTGCCGGCATCAGCACTGCCTCTGGCATCCCCGACTTCAGGGGTCCCCACGGAGTCTGGACCATGG
AGGAGCGAGGTCTGGCCCCCAAGTTCGACACCACCTTTGAGAGCGCGCGGCCCACGCAGACCCACATGGC
GCTGGTGCAGCTGGAGCGCGTGGGCCTCCTCCGCTTCCTGGTCAGCCAGAACGTGGACGGGCTCCATGTG
CGCTCAGGCTTCCCCAGGGACAAACTGGCAGAGCTCACGGGGAACATGTTTGTGGAAGAATGTGCCAAGT
CTTTTTATTTCTATTAAACGTTCTGCACTGGCAAAAA
MECP2
SEQ ID NO:112
>gi | 7710148 | ref | NM_004992.2 | Homo sapiens methyl CpG binding protein
2 (Rett syndrome) (MECP2), mRNA
CCGGAAAATGGCCGCCGCCGCCGCCGCCGCGCCGAGCGGAGGAGGAGGAGGAGGCGAGGAGGAGAGACTG
ATTCGGGGGCGCGAGCTGCCCCAGTAAATGGAAATGTTTTCTAACATATAAAAACCTACAGAAGAAGAAA
ATAATTTTCTGGATCAAATTAGAAGTCTGTATTATATTGATGTCTCCAGATTCAAATATATTAGAAAGCA
GCCGTGGAGACAACCATCTTCATTTTGGGCGAAATAACTAAAGCCCGCCTCAAGCATTAGAACTACAGAC
AAACCCTGATGCGACCTCTCCAGATTGTCCCAAGTCGATTGATTTCCCAGCTATATTGTGGCCTGAAGCC
TCCAGCGTCCACACGAAACCAGATTTGCCTGAAAATGGCTCGGCCAAGTTCAAGTATGGCAGATTTTCGA
AAGTTTTTTGCAAAAGCAAAGCACATAGTCATCATCTCAGGAGCTGGTGTTAGTGCAGAAAGTGGTGTTC
CGACCTTCAGAGGAGCTGGAGGTTATTGGAGAAAATGGCAAGCCCAGGACCTGGCGACTCCCCTGGCCTT
TGCCCACAACCCGTCCCGGGTGTGGGAGTTCTACCACTACCGGCGGGAGGTCATGGGGAGCAAGGAGCCC
AACGCCGGGCACCGCGCCATAGCCGAGTGTGAGACCCGGCTGGGCAAGCAGGGCCGGCGAGTCGTGGTCA
TCACCCAGAACATCGATGAGCTGCACCGCAAGGCTGGCACCAAGAACCTTCTGGAGATCCATGGTAGCTT
ATTTAAAACTCGATGTACCTCTTGTGGAGTTGTGGCTGAGAATTACAAGAGTCCAATTTGTCCAGCTTTA
TCAGGAAAAGGTGCTCCAGAACCTGGAACTCAAGATGCCAGCATCCCAGTTGAGAAACTTCCCCGGTGTG
AAGAGGCAGGCTGCGGGGGCTTGCTGCGACCTCACGTCGTGTGGTTTGGAGAAAACCTGGATCCTGCCAT
TCTGGAGGAGGTTGACAGAGAGCTCGCCCACTGTGATTTATGTCTAGTGGTGGGCACTTCCTCTGTGGTG
TACCCAGCAGCCATGTTTGCCCCCCAGGTGGCTGCGAGGGGCGTGCCAGTGGCTGAATTTAACACGGAGA
CCACCCCCAGCTACGAACAGATTCAGTCATTTGATCTCCATCTCATCTCTAATTATTATAAAGAATTAAA
TAGAAGAGATGCTGGTGATAGTCATCTCATCCTACTCAATTTTTCAAAGGCAGAAACCAACCCTGGAGCA
CCACCCCAGCTACGAACAGATTCAGTCATTTGATCTCCATCTCATCTCTAATTATTATAAAGAATTAAAA
CAAGTCATCATTGTAGAAAAGCAAGAAAATGCAGATAGAGAAAAAGAAGAAAAATAAAACTGGAGTATTC
CTGTAAATAAGGTTTCATTGGAACACAGCTGTGGCCATATGTTTGTATATTGTGTGTGGCTGCTTTTGCA
TTAGGATGACAGAGGTGAATAGTTGCAACAGAGACTGGCTGGTCTGCAAGCCTAAATATATGTCCAATGT
TGCTACTTTTAACCGATTTTCTTGTACCTAAATAGGCAGCTAGATTGCTGCCTATATTTTAAATATCCGG
ATTTGGATCTCACAAGACAACCTTAGGCCTACACAGGTTGTTCAGATTCTTTGCCCCAGTTCTAATTAGT
GACAAAGGCATAGAATTCTCCTCCACAGGAATGTATTTCTATTTTCAAGGTGATTAATTAGTTCCAGTTT
CGACAAGCACAGTCAGGTTGAAGACCTAACCAGGGCCAGAAGTAGCTTTGCACTTTTCTAAACTAGGCTC
CTTCAACAAGGCTTGCTGCAGATACTACTGACCAGACAAAGCTGTTGACCAGGCACCTCCCCTCCACCCA
TGCAACAAAACCCTAGCAGCCTGAAGAACTCTAAGCCAGATGGGGTGGACGAGAGCAGAGGCCTCTTGGC
TCAGCAAAGAATGCACAGTATGATCAGCTCAGTGGATGTGAAGTCAGAAGTTCCTGTGGGCCTGGAGCCC
ATCTCACCTTTAGACCTAAGGACAGACCTCAGGATGATGATGCCCGTGGTGGACCCTGTTGTCCTGAAGA
AGCAATTGCAGCAGGAATTACTTCTTATCCAGCAGCAGCAACAAATCCAGAAGCAGCTTCTGATAGCAGA
GTTTCAGAAACAGCATGAGAACTTGACACGGCAGCACCAGGCTCAGCTTCAGGAGCATATCAAGGAACTT
CTAGCCATAAAACAGCAACAAGAACTCCTAGAAAAGGAGCAGAAACTGGAGCAGCAGAGGCAAGAACAGG
AAGTAGAGAGGCATCGCAGAGAACAGCAGCTTCCCTCCTCTCAGAGGCAAGATAGAGGACGAGAAAGGGC
AGTGGCAAGTACAGAAGTAAAGCAGAAGCTTCAAGAGTTCCTACTGAGTAAATCAGCAACGAAAGACACT
CCAACTAATGGAAAAAATCATTCCGTGAGCCGCCATCCCAAGCTCTGGTACACGGCTGCCCACCACACAT
CATTGGATCAAAGCTCTCCACCCCTTAGTGGAACATCTCCATCCTAACAGTACACATTACCAGGAGCACA
AGATGCAAAGGATGATTTCCCCCTTCGAAAAACTGCCTCTGAGCCCAACTTGAAGGTGCGGTCCAGGTTA
AAACAGAAAGTGGCAGAGAGGAGAAGCAGCCCCTTACTCAGGCGGAAGGATGGAAATGTTGTCACTTCAT
TCAAGAAGCGAATGTTTGAGGTGACAGAATCCTCAGTCAGTAGCAGTTCTCCAGCGCTCTGGTCCCAGTT
ACCAAACAATGGGCCAACTGGAAGTGTTACTGAAAAATGAGACTTCGGTTTTGCCCCCTACCCCTCATGC
GAGCAAATGGTTTCACAGCAACGCATTCTAATTCATGAAGATTCCATGAACCTGCTAAGTCTTTATACCT
CTCCTTCTTTGCCCAACATTACCTTGGGGCTTCCCGCAGTGCCATCCCAGCTCAATGCTTCGAATTCACT
CAAAGAAAAGCAGAAGTGTGAGACGCAGACGCTTAGGCAAGGTGTTCCTCTGCCTGGGCAGTATGGAGGC
AGCATCCCGGCATCTTCCAGCCACCCTCATGTTACTTTAGAGGGAAAGCCACCCAACAGCAGCCACCAGG
CTCTCCTGCAGCATTTATTATTGAAAGAACAAATGCGACAGCAAAAGCTTCTTGTAGCTGGTGGAGTTCC
CTTACATCCTCAGTCTCCCTTGGCAACAAAAGAGAGAATTTCACCTGGCATTAGAGGTACCCACAAATTG
CCCCGTCACAGACCCCTGAACCGAACCCAGTCTGCACCTTTGCCTCAGAGCACGTTGGCTCAGCTGGTCA
TTCAACAGCAACACCAGCAATTCTTGGAGAAGCAGAAGCAATACCAGCAGCAGATCCACATGAACAAACT
GCTTTCGAAATCTATTGAACAACTGAAGCAACCAGGCAGTCACCTTGAGGAAGCAGAGGAAGAGCTTCAG
GGGGACCAGGCGATGCAGGAAGACAGAGCGCCCTCTAGTGGCAACAGCACTAGGAGCGACAGCAGTGCTT
GTGTGGATGACACACTGGGACAAGTTGGGGCTGTGAAGGTCAAGGGGAACCAGTGGACAGTGATGAAGAA
TGCTCAGATCCAGGAAATGGAATCTGGGGAGCAGGCTGCTTTTATGCAACAGGTAATAGGCAAAGATTTA
GCTCCAGGATTTGTAATTAAAGTCATTATCTGAACATGAAATGCATTGCAGGTTTGGTAAATGGATATGA
TTTCCTATCAGTTTATATTTCTCTATGATTTGAGTTCAGTGTTTAAGGATTCTACCTAATGCAGATATAT
GTATATATCTATATAGAGGTCTTTCTATATACTGATCTCTATATAGATATCAATGTTTCATTGAAAATCC
ACTGGTAAGGAAATACCTGTTATACTAAAATTATGATACATAATATCTGAGCAGTTAATAGGCTTTAAAT
TTATCCCAAAGCCTGCTACACCAATTACTTCTAAAGAAAACAAATTCACTGTTATTTTGAGTTTATGTGT
TGAGATCAGTGACTGCTGGATAGTCTCCCAGTCTGATCAATGAAGCATTCGATTAGTTTTTGATTTTTTG
CAACATCTAGAATTTAATTTTCACATCACTGTACATAATGTATCATACTATAGTCTTGAACACTGTTAAA
GGTAGTCTGCCCCTTCCTTCCTCTCTCTTTTTTTAGTTAAGTAGAAATGTTCTGGTCACCATGCCAGTAG
TCCTAGGTTATTGTGTAGGTTGCAATTGAACATATTAGGAATACAGGTGGTTTTAAATATATAGATGCAA
ATTGCAGCACTACTTTAAATATTAGATTATGTCTCACATAGCACTGCTCATTTTACTTTTATTTTGTGTA
ATTTGATGACACTGTCTATCAAAAAAGAGCAAATGAAGCAGATGCAAATGTTAGTGAGAAGTAATGTGCA
GCATTATGGTCCAATCAGATACAATATTGTGTCTACAATTGCAAAAAACACAGTAACAGGATGAATATTA
TCTGATATCAAGTCAAAATCAGTTTGAAAAGAAGGTGTATCATATTTTATATTGTCACTAGAATCTCTTA
AGTATAATTCCATAATGACATGGGCATATACCGTAACATTCTGGCAAATAACAATTAGAAAAGATAGGTT
TAACAAAAAAATTTACTTGTATATAATGCACCTTCAGGAGGACTATGTCCTTTGATGCTATAAAATACAA
ACAACTTTGAAGGCAACAGAAGACACTGTTTATTCAAGTCAGTTCTTTGTCAGGTTCCTGCTGTTCTCCT
ACAGAAAAGTGATTCTGTGAGGGTGAACAGGAAATGCCTTGTGGAAACAGGAAGTCCAAGTGATTCATGT
ACTGAGGAATGTAGGAAAAAAATCTGAGGATAGTGCTTTACTCTTTCTGTTTTTTAAAGGGCACTCTATG
AATTGATTTATTGTCTAAGAAAATAACACCACAAGTAGGGAAATTGTTACGGAAGCTTTTCACTGGAACA
TTTCCTTCATATTCCCTTTTGATATGTTTACCTTGTTTTATAGGTTTACTTTTGTTAAGCTAGTTAAAGG
TTCGTTGTATTAAGACCCCTTTAATATGGATAATCCAAATTGACCTAGAATCTTTGTGAGGTTTTTTCTA
TTAAAATATTTATATTTCTAAATCCGAGGTATTTCAAGGTGTAGTATCCTATTTCAAAGGAGATATAGCA
GTTTTGCCAAATGTAGACATTGTTCACTGTATGTTATTGGCACGTGTTGTTTACATTTTGCAGTGAAATT
TTAAAAAATATTTCTTTAAAAATGTTACTGCTAAAGATACATTATCCTTTTTTAAAAAGTCTCCATTCAA
TTAAATTAACATAACTAGAAGTTAGAAAGTTTAAAAGTTTTCCACATAATGAAAGTCCTTCTGATAATTT
GACAAATAGCTATAATAGGAACACTCCCTATCACCAACATATTTTGGTTAGACGTATATTCCTTTTCCAT
ATGACTTTTTGTCAGTTGTTTTGCATTAAAAATATGGCATGCCTAAGATAAAATTGTATATTTTTACCAT
CTCATAAATATTCATTTTCTTCAAAGTCTTTTTTTCAATCTCATAAAAAAGGGATAGTGCATCTTTTAAA
TACATTTTATTTGGGGAGGAACATGTGGCTGAGCAGACTTTTGTATAATATTACTTCAAAGATATGTAAT
CACAAACAAAAAAAACTATTTTTTATAATGTCATTTGAGAGAGTTTCATCAGTACAGTTGGTGGACGTTA
ATTGTTTGAATTTGATAGTCTTTGAATTTAATCAAGAAACTACCTGGAACCAGTGAAAAGGAAAGCTTGA
CTTAAATAATCTTAGAATTAATTGATAAATGTCTCTTTTAAAATCTACTGTATTTATTATAATTTACACC
ACCCTCCTTCCCACTCTTCTCATCTTCGACAGAGCCTGCCCCAGCGCTGACGTGTCAGGAAACACCCAGA
GAACTAGGAAGGCACTTCTGCCTGAGGGGCAGCCTGCCTTGCCCACTCCTGCTCTGCTCGCCTCGGATCA
GCTGAGCCTTCTGAGCTGGCCTCTCACTGCCTCCCCAAGGCCCCCTGCCTGCCCTGTCAGGAGGCAGAAG
GAAGCAGGTGTGAGGGCAGTGCAAGGAGGGAGCACAACCCCCAGCTCCCGCTCCGGGCTCCGACTTGTGC
ACAGGCAGAGCCCAGACCCTGGAGGAATCCTACCCTTTGAATTCAAGAACATTTGGGGAATTTGGAAATC
TCTTTGCCCCCAAACCCCCATTCTGTCCTACCTTTAATCAGGTCCTGCTCAGCAGTGAGAGCAGATGAGG
TGAAAAGGCCAAGAGGTTTGGCTCCTGCCCACTGATAGCCCCTCTCCCCGCAGTGTTTGTGTGTCAAGTG
GCAAAGCTGTTCTTCCTGGTGACCCTGATTATATCCAGTAACACATAGACTGTGCGCATAGGCAAGCTTT
GTCTCCTCTATCCTGGGCTTTGTTTTGCTTTATTAGTTTTGCTTTTAGTTTTTCTGTCCCTTTTATTTAA
GGTCAGGGTGAGCCCATGTGGAGTCAGCCTCGCAGGCCTCCCTGCCAGTAGGGTCCGAGTGTGTTTCATC
CTTCCCACTCTGTCGAGCCTGGGGGCTGGAGCGGAGACGGGAGGCCTGGCCTGTCTCGGAACCTGTGAGC
TGCACCAGGTAGAACGCCAGGGACCCCAGAATCATGTGCGTCAGTCCAAGGGGTCCCCTCCAGGAGTAGT
GAAGACTCCAGAAATGTCCCTTTCTTCTCCCCCATCCTACGAGTAATTGCATTTGCTTTTGTAATTCTTA
ATGAGCAATATCTGCTAGAGAGTTTAGCTGTAACAGTTCATTTTGATCATCTTTTTTTAATAATTAGAAA
CACCAAAAATGCCAGAAACTTGTTCTTCCATTAGCAGAGAGCATTATAATCACCAGGGCCAAAAGCTTCC
CTCCCTGGTGTCATTGCTTCTTCTGAGGCCTGAATCCAAAAGAAAAACAGCCATAGGCCCTTTCAGTGGC
CGGGCTACCCGTGAGCCCTTCGGAGGACCAGGGCTGGGGCAGCCTCTGGGCCCACATCCGGGGCCAGCTC
CGGCGTGTGTTCAGTGTTAGCAGTGGGTCATGATGCTCTTTCCCACCCAGCCTGGGATAGGGGCAGAGGA
GGCGAGGAGGCCGTTGCCGCTGATGTTTGGCCGTGAACAGGTGGGTGTCTGCGTGCGTCCACGTGCGTGT
TTTCTGACTGACATGAAATCGACGCCCGAGTTAGCCTCACCCGGTGACCTCTAGCCCTGCCCGGATGGAG
CGGGGCCCACCCGGTTCAGTGTTTCTGGGGAGCTGGACAGTGGAGTGCAAAAGGCTTGCAGAACTTGAAG
CCTGCTCCTTCCCTTGCTACCACGGCCTCCTTTCCGTTTGATTTGTCACTGCTTCAATCAATAACAGCCG
CTCCAGAGTCAGTAGTCAATGAATATATGACCAAATATCACCAGGACTGTTACTCAATGTGTGCCGAGCC
CTTGCCCATGCTGGGCTCCCGTGTATCTGGACACTGTAACGTGTGCTGTGTTTGCTCCCCTTCCCCTTCC
TTCTTTGCCCTTTACTTGTCTTTCTGGGGTTTTTCTGTTTGGGTTTGGTTTGGTTTTTATTTCTCCTTTT
GTGTTCCAAACATGAGGTTCTCTCTACTGGTCCTCTTAAGTGTGGTGTTGAGGCTTATATTTGTGTAATT
TTTGGTGGGTGAAAGGAATTTTGCTAAGTAAATCTCTTCTGTGTTTGAACTGAAGTCTGTATTGTAACTA
TGTTTAAACTAATTGTTCCAGAGACAAATATTTCTAGACACTTTTTCTTTACAAACAAAAGCATTCGGAG
GGAGGGGGATGGTGACTGAGATGAGAGGGGAGAGCTGAACAGATGACCCCTGCCCAGATCAGCCAGAAGC
CACCCAAAGCAGTGGAGCCCAGGAGTCCCACTCCAAGCCAGCAAGCCGAATAGCTGATGTGTTGCCACTT
TCCAAGTCACTGCAAAACCAGGTTTTGTTCCGCCCAGTGGATTCTTGTTTTGCTTCCCCTCCCCCCGAGA
TTATTACCACCATCCCGTGCTTTTAAGGAAAGGCAAGATTGATGTTTCCTTGAGGGGAGCCAGGAGGGGA
TGTGTGTGTGCAGAGCTGAAGAGCTGGGGAGAATGGGGCTGGGCCCACCCAAGCAGGAGGCTGGGACGCT
CTGCTGTGGGCACAGGTCAGGCTAATGTTGGCAGATGCAGCTCTTCCTGGACAGGCCAGGTGGTGGGCAT
AACCTGTCTGAGGTTGGGAGAGGTGCACTTGGGGCACAGGGAGAGGCCGCGACACACTTAGCTGGAGATG
TCTCTAAAAGCCCTGTATCGTATTCACCTTCAGTTTTTGTGTTTTGGGACAATTACTTTAGAAAATAAGT
AGGTCGTTTTAAAACAAAAATTATTGATTGCTTTTTTTGTAGTGTTCAGAAAAAAGGTTCTTTGTGTATA
GCCAAATGACTGAAAAAGCACTGATATATTTACAAATTGGCAAATTTATTAAGGAAATTTGTACCATTTC
AGTAAACCTGTCTGAATGTACCTGTATACGTTTCAAAAACACCCCCCCCCCACTGAATCCCTGTAACCTA
TTTATTATATAAAGAGTTTGCCTTATAAATTT
ZNF145
SEQ ID NO:113
>gi | 5174752 | ref | NM_006006.1 | Homo sapiens zinc finger
protein 145 (Kruppel-like, expressed in promyelocytic
leukemia) (ZNF145), mRNA
CAGGAAGCCCACCCAGCCCCGCCACGCAGAGCCCAGAAGGAAAGAAAGCCTCATGCCTGAGCCGAGGGGA
GCACCATGGATGTGACAAAAATGGGCATGATCCAGCTGCAGAACCCTAGCCACCCCACGGGGCTACTGTG
CAAGGCCAACCAGATGCGGCTGGCCGGGACTTTGTGCGATGTGGTCATCATGGTGGACAGCCAGGAGTTC
CACGCCCACCGGACGGTGCTGGCCTGCACCAGCAAGATGTTTGAGATCCTCTTCCACCGCAATAGTCAAC
ACTATACTTTGGACTTCCTCTCGCCAAAGACCTTCCAGCAGATTCTGGAGTATGCATATACAGCCACGCT
GCAAGCCAAGGCGGAGGACCTGGATGACCTGCTGTATGCGGCCGAGATCCTGGAGATCGAGTACCTGGAG
GAACAGTGCCTGAAGATGCTGGAGACCATCCAGGCCTCAGACGACAATGACACGGAGGCCACCATGGCCG
ATGGCGGGGCCGAGGAAGAAGAGGACCGCAAGGCTCGGTACCTCAAGAACATCTTCATCTCGAAGCATTC
CAGCGAGGAGAGTGGGTATGCCAGTGTGGCTGGACAGAGCCTCCCTGGGCCCATGGTGGACCAGAGCCCT
TCAGTCTCCACTTCATTTGGTCTTTCAGCCATGAGTCCCACCAAGGCTGCAGTGGACAGTTTGATGACCA
TAGGACAGTCTCTCCTGCAGGGAACTCTTCAGCCACCTGCAGGGCCCCGAGGAGCCAACTCTGGCTGGGG
TGGGCGGCACCCTGGGGTGGCTGAGGTGAAGACGGAGATGATGCAGGTGGATGAGGTGCCCAGCCAGGAC
AGCCCTGGGGCAGCCGAGTCCAGCATCTCAGGAGGGATGGGGGACAAGGTTGAGGAAAGAACAAAGAGGG
GGCCTGGGACCCCGACTCGAAGCAGCGTCATCACCAGTGCTAGGGAGCTACACTATGGGCGAGAGGAGAG
TGCCGAGCAGGTGCCACCCCCAGCTGAGGCTGGCCAGGCCCCCACTGGCCGACCTGAGCACCCGCAACCC
CCGCCTGAGAAGCATCTGGGCATCTACTCCGTGTTGCCCAACCACAAGGCTGACGCTGTATTGACGATGC
CGTCTTCCGTGACCTCTGGCCTCCACGTGCAGCCTGCCCTGGCTGTCTCCATGGACTTCAGCACCTATGG
GGGGGCTGCTGCCCCAGGGCTTCATCCAGAGGGAGCTGTTCAGCAAGCTGGGGAGCTGGCTGTGGGCATG
AAGTCAGAGAGCCGGACCATCGGAGAGCAGTGCAGCGTGTGTGGGGTCGAGCTTCCTGATAACGAGGCTG
TGGAGCAGCACAGGAAGCTGCACAGTGGGATGAAGACGTACGGGTGCGAGCTCTGCGGGAAGCGGTTCCT
GGATAGTTTGCGGCTGAGAATGCACTTACTGGCTCATTCAGCGGGTGCCAAAGCCTTTGTCTGTGATCAG
TGCGGTGCACAGTTTTCGAAGGAGGATGCCCTGGAGACACACAGGCAGACCCATACTGGCACTGACATGG
CCGTCTTCTGTCTGCTGTGTGGGAAGCGCTTCCAGGCGCAGAGCGCACTGCAGCAGCACATGGAGGTCCA
CGCGGGCGTGCGCAGCTACATCTGCAGTGAGTGCAACCGCACCTTCCCCAGCCACACGGCTCTCAAACGC
CACCTGCGCTCACATACAGGCGACCACCCCTACGAGTGTGAGTTCTGTGGCAGCTGCTTCCGGGATGAGA
GCACACTCAAGAGCCACAAACGCATCCACACGGGTGAGAAACCCTACGAGTGCAATGGCTGTGACAAGAA
GTTCAGCCTCAAGCATCAGCTGGAGACGCACTATAGGGTGCACACAGGTGAGAAGCCCTTTGAGTGTAAG
CTCTGCCACCAGCGCTCCCGGGACTACTCGGCCATGATCATGCACGTGAGAACGCACAACGGCGCCTCGC
CCTACCAGTCACCATCTGACAGAGTACTGCCCCAGCCTCTCCTCCATGAGAAGCACATGAAGCAGGGCCA
CAAGCCCGAGGAGATCCCGCCCGACTGGAGGATAGAGAAGACGTACCTCTACCTGTGCTATGTGTGAAGG
GAGGCCCGCGGCGGTGGAGCCGAGCGGGGAGCCAGGATAGAAGAGTTGGAGTGAGATGAAGGAAGGACTA
TFDP1
SEQ ID NO:114
>gi | 6005899 | ref | NM_007111.1 | Homo sapiens transcription factor Dp-1
(TFDP1), mRNA
GGAATTCCGTAGCTATTGATTTCCCGGATCTGGTAACATGGCAAAAGATGCCGGTCTAATTGAAGCCAAC
GGAGAACTCAAGGTCTTCATAGACCAGAACCTTAGTCCCGGGAAAGGCGTGGTGTCCCTCGTGGCCGTTC
ACCCCTCCACCGTCAACCCGCTCGGGAAGCAGTCTTGCCATTACCTTTGGACAGTCCAAAATGTCAACAT
TGCCCAGCAAGTGGTAATTGGTACGCCTCAGAGACCCGCAGCGTCAAACACCCTGGTGGTAGGAAGCCCA
GCAACAGGAAAGGAGAGAAGAATGGCAAGGGCCTACGGCATTTCTCCATGAAGGTCTGCGAGAAGGTGCA
GAGGAAAGGGACCACTTCCTACAACGAAGTGGCAGACGAGCTGGTTGCGGAGTTCAGTGCTGCCGACAAC
CACATCTTACCAACGAGTCAGCTTATGACCAGAAAGACATAGACGGCGCGCGTCTACGATGCCTTAAACG
TGCTAATGGCCATGAACATCATCTCCAAGGAGAAGAAGGAGATCAAGTGGATTGGTCTGCCCACCAACTC
GGCTCAGGAATGTCAGAACTTAGAGGTGGAAAGACAGAGGAGACTTGAAAGAATAAAACAGAAACAGTCT
CAACTTCAAGAACTTATTCTACAGCAAATTGCCTTCAAGAACCTGGTGCAGAGAAAGCGGCATGCGGAGC
AGCAGGCCAGCCGGCCACCGCCACCCAACTCAGTCATCCACCTGCCCTTCATCATCGTCAACACCAGCAA
GAAGACGGTCATCGACTGCAGCATCTCCAATGACATTCAGTATCTGTTTTTTATATTTGACAACACATTT
GAAATCCACGATGACATAGAAGTGCTGAAGCGGATGGGCATGGCTTGCGGGCTGGAGTCGGGGAGCTGCT
CTGCCGAAGACCTTAAAATGGCCAGAAGTCTGGTCCCCAAGGCTCTGGAGCCATACGTGACAGAAATGGC
TCAGGGAACTGTTGGAGGCGTGTTCATCACGACGGCAGGTTCCACGTCTAACGGCACAAGGTTCTCTGCC
AGTGACCGACCAACGGTGCAGATGGGATGCTGGCCCAAAGACTCCAATGGGTCTCAGTACAGCGGCTCCA
GGGTGGAGACTCCGGTGTCCTACGTCGGGGAGGACGACGAGGAGGACGATGACTTCAACGAGAATGACGA
GGACGACTGACGTCCTCCCCACTTCAGATTCGGCTTCAGGAAAACGTTTAGCGAAAAGAPACTTTTTTTT
TAATGTGGGTTTTCTGTTTCCTTTTGGCCTAGTCCCAAGAAGATATTGGTAAGCTATTGAATTTAGATAT
GCACCTCTGATAAGCAAGGATTGTTTCCCGTAGATTAGGA
SAP30
SEQ ID NO:115
>gi | 4506782 | ref | NM_003864.1 | Homo sapiens sin3-associated
polypeptide, 30kD (SAP30), mRNA
CCCCCATGTGACAGTGACGGGGTCCCCGCTCCAGGAGACGCTCGAGTCTGCGTCCCGGCCCTCAGCACTG
TCCACTGTTTCGGTGCCAGCAGAGACCAGCAGGCCCGGGACAGTTGGTGTTTGGCCGTGCCGCTGTCTAA
CTTGGTGTGCAGAGTGATTTGCCGCTGCCGGAGCGGAGAGAGGCGGAGCGGCCAGGAGAGAGGGGATTTC
TGTCAGCGCCGGCCTCGGGAGCTCGGAGACATGAACGGCTTCACGCCTGACGAGATGAGCCGCGGCGGGG
ATGCGGCCGCCGCAGTGGCCGCAGTGGTCGCTCCGCGGCCGCCGCCGCCTCGGCGGGGAAACGGGACAAG
CGCGGGCACCGGGGCTGAGGTGCCGGGCGCGGGGGCGGTCTCAGCGGGGCCCCCGGGGGCCGGGCCCGGG
CCGGGCCCCGGGCAACTGTGCTGCCTGCGGGAGGATGGTGAGCGGTGCGGCCGGGCGGCAGGCAACGCCA
GCTTCAGCAAGAGGATCCAGAAGAGCATCTCCCAGAAGATGGTGAAGATCGAGCTGCATAAGAGCGCAAG
GCATCTTTACATATGTGATTATCVATAAAAACTTAATTCAGAGTGTTCGAAACAGAAGAAAGAGAAAAGG
AGTGATGATGATGGAGGTGATTCACCTGTTCAAGATATTGATACCCCAGAGGTTGATTTATACCAATTAC
AAGTAAATACACTTAGGAGATACAAAAGACACTTCAAGCTACCAACCAGACCAGGACTTAATAAAGCACA
ACTTGTTGAGATAGTTGGTTGCCACTTTAGGTCTATTCCAGTGAATGAAAAAGACACCTTAACATATTTC
ATCTACTCAGTGAAGAATGCAAGAACAAATCAGATCTCAAGGTTGATAGTGGTGTTCACTAGGAGACGTT
GGAATTGAGACTAATAACTTGGATGTTAACACTGTTTACTGTTTTTTCACATGTAGATATGTTCTTTGTG
TATTTTTTCTACAGAGGATTTTCTCTGATTTTATTTTCTTTGTTTCTGACTCTAATAATTAGTTGGAAAC
TCATATAAAATGAGCTTTCCTAAATTAAATCTATTTTAATAAAGGTTATAACTATTAAAAAAAAAAAAAA
AAAAA
SAP18
SEQ ID NO:116
>gi | 12056471 | ref | NM_005870.2 | Homo sapiens sin3-associated
polypeptide, 18kD (SAP18), mRNA
AGGTTTGAACTGACTTTATTATTTTGTAAATGTGAATTTTACAAAGCGCTTTACAATTAATGATCACATC
CTTTTGTTTGTCATGGATTTCCACTGTCTGAACGGCTCTGAGCACGCTTGAAGCCCTCGGTTTCCCCTGT
TCGCTTTTGAATGTTTCAGTTTTAGTTATTGATACAATGTCAGCCATGGCTAAAAAGTAACAGTCTTGAC
TCTACCGAGTAACAGCACAAAAACAGAGTGAGGGCTCAGGAAAACAAAACAAAGGCTTCCTCCTTAAAAA
AAAGACAAAAAAAAAAGCTAAGCATCTGTGGCTGAAATCAACTCAGTGGTACTGTGAAACCAATTCCTTT
ACAGCACAGGAAAATTTATTTTTTAACAGTCGTGAGTTACAGTACTTTAACCCCTAAACAGACTCTTTAA
AACAACCGTCTCCTTTTTAAAAGTCTCTTTTTTCCAAACATTCCATCCGAA0GATGGATGCTCTACTTGC
ACCCAGTGCCATCCAAATGTTCAAGTCAAAAATATTTATACATTTTATACTTAGGCGAGCGTCTCGCAGG
CCGTAGGAGGAAGATGGCGGTGGAGTCGCGCGTTACCCAGGAGGAAATTAAGAAGGAGCCAGAGAAACCG
ATCGACCGCGAGAAGACATGCCCACTGTTGCTACGGGTCTTCACCACCAATAACGGCCGCCACCACCGAA
TGGACGAGTTCTCCCGGGGAAATGTACCGTCCAGCGAGTTGCAGATCTACACTTGGATGGATGCAACTTT
GAAAGAACTGACAAGCTTAGTAAAAGAAGTCTACCCAGAAGCTAGAAAGAAGGGCACTCACTTCAATTTT
GCAATCGTTTTTACAGATGTTAAAAGACCTGGCTATCGAGTTAAGGAGATTGGCAGCACCATGTCTGGCA
GAAAGGGGACTGATGATTCCATGACCCTGCAGTCGCAGAAGTTCCAGATAGGAGATTACTTGGACATAGC
AATTACCCCTCCAAATCGGGCACCACCTCCTTCAGGGCHCATGAGACCATATTAAATTCTATTTACTATT
TGTTGAATTTATTTTTCCGTCAGTTATGTAAAATAAACATACTCTTCTTCCTCCCCTGATTATTGCCATT
AAGCCTTTAAATTCTAAACAAATTATAATGCATCATCTATTTAGGAGTTAGATTTGGATGTGCTATTGTA
TGATTACGAATAGTCTGTATGTTTCAAGCCCTTCTGTAAAATATGAAGAAAAGTCTCTTAGCATTCTGTG
TAAAACTGTACTGTTAAATATATGTGTGTAATC
RBBP7
SEQ ID NO:117
>gi | 13259504 | ref | NM_002893.2 | Homo sapiens retinoblastoma binding
protein 7 (RBBP7), mRNA
GCCTCGTCAGCTGCCTGGGCGGGCTGGGAGGCGCGGGTTGAAAAGTCTCGTTCCAAGTTTGGAGAGAGAG
AGAAGAGCGCCTCAGACCTCGGTACCCGCGAGCGGGGAGGAGGCAGGAAAGAAGGACGCGGCGTCTGGGG
AGCACCCAGGCAGCAAGACGGGGCCCGGGCTTTCGACAGTGGGGAGTGTGACGCGCTTGGGAAAGGCAGG
AGCGCCAGCGGTCGGGCTGCTCTTGGCTAACGAGAGGAGTCCGAGGCGGCGGCGAGGGGCGAACGACCCG
ACGCAAGATGGCGAGTAAAGAGATGTTTGAAGATACTGTGGAGGAGCGTGTCATCAATGAAGAATATAAA
ATCTGGAAGAAGAATACACCGTTTCTATATGACCTGGTTATGACCCATGCTCTTCAGTGGCCCAGTCTTA
CCGTTCAGTGGCTTCCTGAAGTGACTAAACCTGAAGGAAAAGATTATGCCCTTCATTGGCTAGTGCTGGG
GACTCATACGTCTGATGAGCAGAATCATCTGGTGGTTGCGCGACTACATATTCCCAATGATGATGCACAG
TTGATGCTTCCCATTGTGACAGTGACAAGGGTGAATTTGGTGGCTTTGGTTCTGTAAACAGGAAAAATTG
TTTATCCTCTGATTCTAGCACTTTCAAGTGAGCTGTTGCGTACTGTATCATATTGTAGCTATTAGGGAAG
AGAAGAATGTTGCTTAAGAAAGAACATCACCATTGATTTTAAATACAAGTAGCAGGGTATTGCCTTTGAT
AATGTGAAATTAAATCAATCACGAAGGAGAAGTAAACCGTGCTCGTTACATGCCGCAGAATCCTCAGCAT
CATTGCTACAAAAACACCATCTTCTGATGTGTTGGTTTTTTGACTATACAAAACACCCTGCTAAACCAGA
CCAAGTGGAGAATGTAATCCTGATCTCAGATTAAGAGGTCACCAGAAGGAAGGCTATGGTCTCTCCTGGA
ATTCAAATTTGAGTGGACATCTCCTAAGTGCATCTGATGACCATACTGGTTTGTCTGTGGGATATAACGC
AGGACCAAAAGAAGGCAAAATTGTGGATGCTAAAGCCATCTTTACTGGCCACTCAGCTGTTGTAGAGGAT
GTGGCCTGGCAGGTGCTGCACGAGTCATTGTTTGGATCGTTGCTGATGATCAGAAACTTATGATATGGGA
ACACCAGGTCCAATACCACCTCCAAGCCGAGTCACTTGGTGGATGCGCACACTGCCGAAGTCAACTGCCT
CTCATTCAATCCCTACAGCGAATTTATTCTAGCCACCGGCTCTGCGGATAAGACCGTAGCTTTATGGGAT
CTGCGTAACTTAAAATTAAAACTCCATACCTTCGAATCTCATAAAGATGAAATTTTCCAGGTCCACTGGT
CTCCACATAATGAAACTATTCTGGCTTCAAGTGGTACTGACCGCCGCCTGAATGTGTGGGATTTAAGTAA
AATTGGGGAAGAACAATCAGCAGAAGATGCAGAAGATGGCCTCCAGAACTCCTGTTTATTCATTGGAGGA
CACACTGCTAAGATTTCAGTTTTAGCTGGAACCCCAATGAGCCTTGGGTCATTTGCTCAGTGTCTGAGGA
ATAACATCATGCAGATATGGCAAATGGCTGAAAATATTTACAATGATGAAGAGTCAGATGTCACGACATC
CGAACTGGAGGGACAAGGATCTTAAACCCAAAGTACGAGAAATGTTTCTGTTGAATGTAATGCTACATGA
ATGCTTAGATTATCAAGCGCCAAAAGGCATTGTATAGTAGGAAATGTAAGTGGGGTGGCTTAATGGCTTC
TTTATCCTCTGATTCTAGCACTTTCAAGTGAGCTGTTGCGTATGTATCATATTGTAGCTATTAGGGAAGT
AGAAGAATGTTGCTTAAGAAAGAACATCACCATTGATTTAAACTAAGTGCTTGCTGTTCCCAAATATGCA
TCAACTGTTTTAAGTCCTCATTTTCTCAAACTAAGTGCTTGCTGTTCCCAAATATGCAAGAATAACTTTT
ACACTTTTTCCTTCCAACACTTCTTGATTGGCTTTGCAGAAATAAAGTTTTAAAAT
RBBP4
SEQ ID NO:118
>gi | 5032026 | ref | NM_005610.1 | Homo sapiens retinoblastoma binding
protein 4 (RBBP4), mRNA
CGCGCGCACAGAGCGAGCTCTTGCAGCCTCCCCGCCCCTCCCGCAACGCTCGACCCCAGGATTCCCCGGG
CTCGCCTGCCCGCCATGGCCGACAAGGAAGCAGCCTTCGACGACGCAGTGGAAGAACGAGTGATCAACGA
GGAATACAAAATATGGAAAAAGAACACCCCTTTTCTTTATGATTTGGTGATGACCCATGCTCTGGAGTGG
CCCAGCCTAACTGCCCAGTGGCTTCCAGATGTAACCAGACCAGAAGGGAAAGATTTCAGCATTCATCGAC
TTGTCCTGGGGACACACACATCGGATGAACAAAACCATCTTGTTATAGCCAGTGTGCAGCTCCCTAATGA
TGATGCTCAGTTTGATGCGTCACACTACGACAGTGAGAAAGGAGAATTTGGAGGTTTTGGTTCAGTTAGT
GGAAAAATTGAAATAGAAATCAAGATCAACCATGAAGGAGAAGTAAACAGGGCCCGTTATATGCCCCAGA
ACCCTTGTATCATCGCAACAAAGACTCCTTCCAGTGATGTTCTTGTCTTTGACTATACAAAACATCCTTC
TAAACCAGATCCTTCTGGAGAGTGCAACCCAGACTTGCGTCTCCGTGGACATCAGAAGGAAGGCTATGGG
CTTTCTTGGAACCCAAATCTCAGTGGGCACTTACTTAGTGCTTCAGATGACCATACCATCTGCGTGTGGG
ACATCAGTGCCGTTCCAAAGGAGGGAAAAGTGGTAGATGCGAAGACCATCTTTACAGGGCATACGGCAGT
AGTAGAAGATGTTTCCTGGCATCTACTCCATGAGTCTCTGTTTGGGTCAGTTGCTGATGATCAGAAACTT
ATGATTTGGGATACTCGTTCAAACAATACTTCCAAACCAAGCCACTCAGTTGATGCTCACACTGCTGAAG
TGAACTGCCTTTCTTTCAATCVTTATAGTGAGTTCATTCTTGCCACAGGATCAGCTGACAAGACTGTTGC
CTTGTGGGATCTGAGAAATCTGAAACTTAAGTTGCATTCCTTTGAGTCACATAAGGATGAAATATTCCAG
GTTCAGTGGTCACCTCACAATGAGACTATTTTAGCTTCCAGTGGTACTGATCGCAGACTGAATGTCTGGG
ATTTAGTAATTGGAGAGGAACAATCCCCAGAAGATGCAGAAGACGGGCCACCAGAGTTGTTACTGTTTAT
TCATGGTGGTCATACTGCGGAGATATCGATTTVTCCTGGAATCCCAATGAACCTTGGGTGATTTTGTTCT
GTATCAGAAGACAATATCATGCAAGTGTGGCAAATGGCAGAGAACATTTATAATGATGAAGACCCTGAAG
GAAQCGTGGATCCAGAAGGACATGGGTCCTAGATATCTCTTTACTTGTTGTCATTTTAGACTCCCCTTTT
TTCTTCTCAACCCTGAGAGTGATTTAACACTGGTTTTGAGACAGACTTTATTCAGCTATCCCTCTATATA
ATAGGTACCACCGATAATGCTATTAGCCCAAACCGTCGGTTTTTCTAAATATTAATAGGGGGGCTTGATT
CAACAAGCCACAGACTTAACGTTGAAATTTTCTTCAGGAATTTTTCTAGTAACCCAGGTCTAAAAGTAGC
ACAGAAAGGGGAATATTATGTGTGATTATTTTTCTTCTTATGCTATATCCCCAAGTTTTTCAGACTCATT
TAAGTAAGGCTAGAGTGAGTAGGAATAGAGCCAAATGAGGTAGGTGTCCTGAAGCCATGAAGTATAAATA
CTGAAAGATGTCACTTTTATTCAGGAAATAGGGGGAGTTCAAGTCGTATAGATTCCTACTCGAAAATCTT
GACACCTGACTTTCCAGGATGCACATTTTCATACGTAGACCAGTTTCCTCTTGGTTTCTTCAGTTAAGTC
AAAACAACACGTTCCTCTTTCCCCATATATTCATATATTTTTGCTCGTTAGTGTATTTCTTGAGCTGTTT
TCATGTTGTTTATTTCCTGTCTGTGAAATGGTGTTTTTTTTTTTGTTGTTGGTTTTTTTTTTTTTTTTTT
AACTTGGGACCACCAAGTTGTAAAGATGTATGTTTTTACCTGACAGTTATACCAGGTAGACTGTCAAAAG
TTGAGAAGAGTGAATCAATAACTTGTATTTGTTTTAAAAATTAAATTAATCCTTGATAAGAGTTGCTTTT
TTTTTTTAGGAGTTAGTCCTTGACCACTAGTTTGATGCCATCTCCATTTTGGGTGACCTGTTTCACCAGC
AGGCCTGTTACTCTCCATGACTAACTGTGTAAGTGCTTAAAATGGAATAAATTGCTTTTCTACATAAAAA
AAAA
RB1
SEQ ID NO:119
gi | 45064341 | ref | NM_000321.1 | Homo sapiens retinoblastoma 1 (including
osteosarcoma) (RB1), mRNA
TTCCGGTTTTTCTCAGGGGACGTTGAAATTATTTTTGTAACGGGAGTCGGGAGAGGACGGGGCGTGCCCC
GCGTGCGCGCGCGTCGTCCTCCCCGGCGCTCCTCCACAGCTCGCTGGCTCCCGCCGCGGAAAGGCGTCAT
AGCGCCAGCGGTCGGGCTGCTCTTGGCTAACGAGAGGAGTCCGAGGCGGCGGCGAGGGGCGAACGACCCG
ACGCAAGATGGCGAGTAAAGAGATGTTTGAAGATACTGTGGAGGAGCGTGTCATCAATGAAGAATATAAA
ATCTGGAAGAAGAATACACCGTTTCTATATGACCTGGTTATGACCCATGCTCTTCAGTGGCCCAGTCTTA
CCGTTCAGTGGCTTCCTGAAGTGACTAAACCTGAAGGAAAAGATTATGCCCTTCATTGGCTAGTGCTGGG
GACTCATACGTCTGATGAGCAGAATCATCTGGTGGTTGCGCGACTACATATTCCCAATGATGATGCACAG
TTGATGCTTCCCATTGTGACAGTGACAAGGGTGAATTTGGTGGCTTTGGTTCTGTAAACAGGAAAAATTG
TTTATCCTCTGATTCTAGCACTTTCAAGTGAGCTGTTGCGTACTGTATCATATTGTAGCTATTAGGGAAG
AGAAGAATGTTGCTTAAGAAAGAACATCACCATTGATTTTAAATACAAGTAGCAGGGTATTGCCTTTGAT
AATGTGAAATTAAATCAATCACGAAGGAGAAGTAAACCGTGCTCGTTACATGCCGCAGAATCCTCAGCAT
CATTGCTACAAAAACACCATCTTCTGATGTGTTGGTTTTTTGACTATACAAAACACCCTGCTAAACCAGA
CCAAGTGGAGAATGTAATCCTGATCTCAGATTAAGAGGTCACCAGAAGGAAGGCTATGGTCTCTCCTGGA
ATTCAAATTTGAGTGGACATCTCCTAAGTGCATCTGATGACCATACTGGTTTGTCTGTGGGATATAACGC
AGGACCAAAAGAAGGCAAAATTGTGGATGCTAAAGCCATCTTTACTGGCCACTCAGCTGTTGTAGAGGAT
GTGGCCTGGCAGGTGCTGCACGAGTCATTGTTTGGATCGTTGCTGATGATCAGAAACTTATGATATGGGA
ACACCAGGTCCAATACCACCTCCAAGCCGAGTCACTTGGTGGATGCGCACACTGCCGAAGTCAACTGCCT
CTCATTCAATCCCTACAGCGAATTTATTCTAGCCACCGGCTCTGCGGATAAGACCGTAGCTTTATGGGAT
CTGCGTAACTTAAAATTAAAACTCCATACCTTCGAATCTCATAAAGATGAAATTTTCCAGGTCCACTGGT
CTCCACATAATGAAACTATTCTGGCTTCAAGTGGTACTGACCGCCGCCTGAATGTGTGGGATTTAAGTAA
AATTGGGGAAGAACAATCAGCAGAAGATGCAGAAGATGGCCTCCAGAACTCCTGTTTATTCATTGGAGGA
CACACTGCTAAGATTTCAGTTTTAGCTGGAACCCCAATGAGCCTTGGGTCATTTGCTCAGTGTCTGAGGA
ATAACATCATGCAGATATGGCAAATGGCTGAAAATATTTACAATGATGAAGAGTCAGATGTCACGACATC
CGAACTGGAGGGACAAGGATCTTAAACCCAAAGTACGAGAAATGTTTCTGTTGAATGTAATGCTACATGA
ATGCTTAGATTATCAAGCGCCAAAAGGCATTGTATAGTAGGAAATGTAAGTGGGGTGGCTTAATGGCTTC
TTTATCCTCTGATTCTAGCACTTTCAAGTGAGCTGTTGCGTATGTATCATATTGTAGCTATTAGGGAAGT
AGAAGAATGTTGCTTAAGAAAGAACATCACCATTGATTTAAACTAAGTGCTTGCTGTTCCCAAATATGCA
CTTTCTTGGAACCCAAATCTCAGTGGGCACTTACTTAGTGCTTCAGATGACCATACCATCTGCGTGTGGG
ACATCAGTGCCGTTCCAAAGGAGGGAAAAGTGGTAGATGCGAAGACCATCTTTACAGGGCATACGGCAGT
AGTAGAAGATGTTTCCTGGCATCTACTCCATGAGTCTCTGTTTGGGTCAGTTGCTGATGATCAGAAACTT
ATGATTTGGGATACTCGTTCAAACAATACTTCCAAACCAAGCCACTCAGTTGATGCTCACACTGCTGAAG
TGAACTGCCTTTCTTTCAATCVTTATAGTGAGTTCATTCTTGCCACAGGATCAGCTGACAAGACTGTTGC
CTTGTGGGATCTGAGAAATCTGAAACTTAAGTTGCATTCCTTTGAGTCACATAAGGATGAAATATTCCAG
GTTCAGTGGTCACCTCACAATGAGACTATTTTAGCTTCCAGTGGTACTGATCGCAGACTGAATGTCTGGG
ATTTAGTAATTGGAGAGGAACAATCCCCAGAAGATGCAGAAGACGGGCCACCAGAGTTGTTACTGTTTAT
TCATGGTGGTCATACTGCGGAGATATCGATTTVTCCTGGAATCCCAATGAACCTTGGGTGATTTTGTTCT
GTATCAGAAGACAATATCATGCAAGTGTGGCAAATGGCAGAGAACATTTATAATGATGAAGACCCTGAAG
GAAQCGTGGATCCAGAAGGACATGGGTCCTAGATATCTCTTTACTTGTTGTCATTTTAGACTCCCCTTTT
TTCTTCTCAACCCTGAGAGTGATTTAACACTGGTTTTGAGACAGACTTTATTCAGCTATCCCTCTATATA
ATAGGTACCACCGATAATGCTATTAGCCCAAACCGTCGGTTTTTCTAAATATTAATAGGGGGGCTTGATT
CAACAAGCCACAGACTTAACGTTGAAATTTTCTTCAGGAATTTTTCTAGTAACCCAGGTCTAAAAGTAGC
ACAGAAAGGGGAATATTATGTGTGATTATTTTTCTTCTTATGCTATATCCCCAAGTTTTTCAGACTCATT
TAAGTAAGGCTAGAGTGAGTAGGAATAGAGCCAAATGAGGTAGGTGTCCTGAAGCCATGAAGTATAAATA
CTGAAAGATGTCACTTTTATTCAGGAAATAGGGGGAGTTCAAGTCGTATAGATTCCTACTCGAAAATCTT
GACACCTGACTTTCCAGGATGCACATTTTCATACGTAGACCAGTTTCCTCTTGGTTTCTTCAGTTAAGTC
AAAACAACACGTTCCTCTTTCCCCATATATTCATATATTTTTGCTCGTTAGTGTATTTCTTGAGCTGTTT
TCATGTTGTTTATTTCCTGTCTGTGAAATGGTGTTTTTTTTTTTGTTGTTGGTTTTTTTTTTTTTTTTTT
AACTTGGGACCACCAAGTTGTAAAGATGTATGTTTTTACCTGACAGTTATACCAGGTAGACTGTCAAAAG
TTAATGTTTCTATCGATTTTGTGCAAAAGCTTCAAATTAAAACAGCTGCATTAGAAAAAAGAGGCGCTTC
TCCCCTCCCCTACACCTAAGGTGTATTTAAACTATCTTGTGTGATTAACTTATTTAGAGATGCTGATAAC
TTAAAATAGGGGATATTTAAGGTAGCTTCAGCTAGCTTTTAGGAAAATCACTTTGTCTAACTCAGAATTA
TTTTTAAAAAGAAATCTGGTCTTGTTAGAAAACAAATTTTTATTTTGTGCTCATTTAAGTTTCAAACTTA
CTATTTTGACAGTTATTTTGATAACAATGACACTAGAAAACTTGACTCCATTTCATCATTGTTTCTGCAT
GATATCATACAATCAGTTAGTTTTTAGGTCAAGGGCTTACTATTTCTGGGTCTTAATTGCTACTAAGTTC
ACATTAGAATTAGTGCCAGAATTTTAGGAACTTCAGAGATCGTGTATTGAGATTTCTTAAATAATGCTTC
AGATATTATTGCTTTATTGCTTTTTTGTATTGGTTAAAACTGTACATTTAAAATTGCTATGTTACTATTT
TCTACAATTAATAGTTTGTCTATTTTAAAATAAATTAGTTGTTAAGAGTC
MEN1
SEQ ID NO:120
>gi | 4557744 | ref | NM_000244.1 | Homo sapiens multiple endocrine
neoplasia I (MEN1), mRNA
GGTGTCCGGAGCCGCGGACCTAGAGATCCCAGAAGCCACAGCGCAGCGGCCCGGCCCGCCACTATTTCCA
GGCTCTGCGGGGCAGGGGCCGCCGCCCACCGCCCGCCGCCATGGGGCTGAAGGCCGCCCAGAAGACGCTG
TTCCCGCTGCGCTCCATCGACGACGTGGTGCGCCTGTTTGCTGCCGAGCTGGGCCGAGAGGAGCCGGACC
TGGTGCTCCTTTCCTTGGTGCTGGGCTTCGTGGAGCATTTTCTGGCTGTCAACCGCGTCATCCCTACCAA
CGTTCCCGAGCTCACCTTCCAGCCCAGCCCCGCCCCCGACCCGCCTGGCGGCCTCACCTACTTTCCCGTG
GCCGACCTGTCTATCATCGCCGCCCTCTATGCCCGCTTCACCGCCCAGATCCGAGGCGCCGTCGACCTGT
CCCTCTATCCTCGAGAAGGGGGTGTCTCCAGCCGTGAGCTGGTGAAGAAGGTCTCCGATGTCATATGGAA
CAGCCTCAGCCGCTCCTACTTCAAGGATCGGGCCCACATCCAGTCCCTCTTCAGCTTCATCACAGGCACC
AAATTGGACAGCTCCGGTGTGGCCTTTGCTGTGGTTGGGGCCTGCCAGGCCCTGGGTCTCCGGGATGTCC
ACCTCGCCCTGTCTGAGGATCATGCCTGGGTAGTGTTTGGGCCCAATGGGGAGCAGACAGGTGAGGTCAC
CTGGCACGGCAAGGGCAACGAGGACCGCAGGGGCCAGACAGTCAATGCCGGTGTGGCTGAGCGGAGCTGG
CTGTACCTGAAAGGATCATACATGCGCTGTGACCGCAAGATGGAGGTGGCGTTCATGGTGTGTGCCATCA
ACCCTTCCATTGACCTGCACACCGACTCGCTGGAGCTTCTGCAGCTGCAGCAGAAGCTGCTCTGGCTGCT
CTATGACCTGGGACATCTGGAAAGGTACCCCATGGCCTTAGGGAACCTGGCAGATCTAGAGGAGCTGGAG
CCCACCCCTGGCCGGCCAGACCCACTCACCCTCTACCACAAGGGCATTGCCTCAGCCAAGACCTACTATC
GGGATGAACACATCTACCCCTACATGTACCTGGCTGGCTACCACTGTCGCAACCGCAATGTGCGGGAAGC
CCTGCAGGCCTGGGCGGACACGGCCACTGTCATCCAGGACTACAACTACTGCCGGGAAGACGAGGAGATC
TACAAGGAGTTCTTTGAAGTAGCCAATGATGTCATCCCCAACCTGCTGAAGGAGGCAGCCAGCTTGCTGG
AGGCGGGCGAGGAGCGGCCGGGGGAGCAAAGCCAGGGCACCCAGAGCCAAGGTTCCGCCCTCCAGGACCC
TGAGTGCTTCGCCCACCTGCTGCGATTCTACGACGGCATCTGCAAATGGGAGGAGGGCAGTCCCACGCCT
GTGCTGCACGTGGGCTGGGCCACCTTTCTTGTGCAGTCCCTAGGCCGTTTTGAGGGACAGGTGCGGCAGA
AGGTGCGCATAGTGAGCCGAGAGGCCGAGGCGGCCGAGGCCGAGGAGCCGTGGGGCGAGGAAGCCCGGGA
AGGCCGGCGGCGGGGCCCACGGCGGGAGTCCAAGCCAGAGGAGCCCCCGCCGCCCAAGAAGCCAGCACTG
GACAAGGGCCTGGGCACCGGCCAGGGTGCAGTGTCAGGACCCCCCCGGAAGCCTCCTGGGACTGTCGCTG
GCACAGCCCGAGGCCCTGAAGGTGGCAGCACGGCTCAGGTGCCAGCACCCGCAGCATCACCACCGCCGGA
GGGTCCAGTGCTCACTTTCCAGAGTGAGAAGATGAAGGGCATGAAGGAGCTGCTGGTGGCCACCAAGATC
AACTCGAGCGCCATCAAGCTGCAACTCACGGCACAGTCGCAAGTGCAGATGAAGAAGCAGAAAGTGTCCA
CCCCTAGTGACTACACTCTGTCTTTCCTCAAGCGGCAGCGCAAAGGCCTCTGAACTACTGGGGACTTCGG
ACCGCTTGTGGGGACCCAGGCTCCGCCTTAGTCCCCCAACTCTGAGCCCATGTTCTGCCCCCAGCCCAAA
GGGGACAGGCCTCACCTCTACCCAAACCCTAGGTTCCCGGTCCCGAGTACAGTCTGTATCAAACCCACGA
TTTTCTCCAGCTCAGAACCCAGGGCTCTGCCCCAGTCGTTAGAATATAGGTCTCTTCTCCCAGAATCCCA
GCCGGCCAATGGAAACCTCACGCTGGGTCCTAATTACCAGTCTTTAAAGGCCCAGCCCCTAGAAACCCAA
GCTCCTCCTCGGAACCGCTCACCTAGAGCCAGACCAACGTTACTCAGGGCTCCTCCCAGCTTGTAGGAGC
TGAGGTTTCACCCTTAACCCAAGGGAGCACAGGTCCCACCTCCAGCCCGGGGAGCCTAGGACCACTCAGC
CCCTAGGAGTATATTTCCGCACTTCAGAATTCCATATCTTGCGAATCCAAGCTCCCTGCCCCAAATAACT
TCAGTCCTGCTTCCAGAATTTGGAAATCCTAGTTTCCTCTCCTTCGTATCCCGAGTCTGGGACACAAAAC
TCCGCCCCCAGCCTATGAGCATCCTGAGCCCCGCCCTCTTCCTGACGAAACTGGCCCCGGATCAGAGCAG
GACCTCCCTTCCGACCCTCTGGGAACCTCCCAGAGGTCCAGCCCATCTCGGAGCATCCCGGAGGAAATCT
GCAGAGGGGTTAGGAGTGGGTGACAAGAGCCTGATCTCTTCCTGTTTTGTACATAGATTTATTTTTCAGT
TCCAAGAAAGATGAATACATTTGTTAAAAAAAAAAAAAAAAA

TABLE 5

Histone Acetyltransferases.

GCM5/PCAF Family.
Gcn5
SEQ ID NO:121
>gi | 4503954 | ref | NM_001487.1 | Homo sapiens GCN5 general control of
amino-acid synthesis 5-like 1 (yeast) (GCN5L1), mRNA
CAGCGGGCACGTGACATGGCCCCGGGGAGCCGAGGTGAGCGTCCAGCTTCCGGAGCCGGAGGGGGCCCGG
CGTACCCAGCCCCCAGCCCGACGTGACCATGCTGTCCCGCCTCCTAAAAGAACACCAGGCCAAGCAGAAT
GAACGCAAGGAGCTGCAGGAAAAGAGGAGGCGAGAGGCTATCACTGCAGCGACATGCCTGACAGAAGCTT
TGGTGGATCACCTCAATGTGGGTGTGGCCCAGGCCTACATGAACCAGAGAAAGCTGGACCATGAGGTGAA
GACCCTACAGGTCCAGGCTGCCCAATTTGCCAAGCAGACAGGCCAGTGGATCGGAATGGTGGAGAACTTC
AACCAGGCACTCAAGGAAATTGGGGATGTGGAGAACTGGGCTCGGAGCATCGAGGTGGACATGCGCACCA
TTGCCACTGCACTGGAATATGTCTACAAAGGGCAGCTGCAGTCTGCCCCTTCCTAGCCCCTGTTCCCTCC
CCAAACCCTATCCCTCCTACCTCACCCGCAGGGGGAAGGAGGGAGGCTGACAAGCCTTGAATAAAACACA
AGCCTCC
GCN5L2
SEQ ID NO:122
>gi | 10835100 | ref | NM_021078.1 | Homo sapiens GCN5 general control of
amino-acid synthesis 5-like 2 (yeast) (GCN5L2), mRNA
CCGCTCTCCGCTGCGGGGGAGGCCATGGCGGAACCTTCCCAGGCCCCGACCCCGGCCCCGGCTGCGCAGC
CCCGGCCCCTTCAGTCCCCAGCACCCTGCCCCAACTCCGACTCCTGCACCCAGCCGGCTTCAGCCCCGAT
TCCGACTCCCACCCCGGCACCAGCCCCTGCCCCAGCTGCAGCCCCAGCCGGCAGCACAGGGACTGGGGGG
CCCGGGGTAGGAAGTGGGGGGGCCGGGAGCGGGGGGGATCCGGCTCGGCCTGGCCTGAGCCAGCAGCAGC
GCGCCAGTCAGAGGAAGGCGCAAGTCCGGGGGCTGCCCCGCGCCAAGAAGCTTGAGAAGCTAGGGGTCTT
CTCGGCTTGCAAGGCCAATGGAACCTGTAAGTGTAATGGCTGGAAAAACCCCAAGCCCCCCACTGCACCC
CGCATAGATCTGCAGCAGCCAGCTGCCAACCTGAGTGAGCTGTGCCGCAGTTGTGAGCACCCCTTGGCTG
ACCACGTATCCCACTTGGAGAATGTGTCAGAGGATGAGATAAACCGACTGCTGGGGATGGTGGTGGATGT
GGAGAATCTCTTCATGTCTGTTCACAAGGAAGAGGACACAGACACCAAGCAGGTCTATTTCTACCTCTTC
AAGCTACTGCGGAAATGCATCCTGCAGATGACCCGGCCTGTGGTGGAGGGGTCCCTGGGCAGCCCTCCAT
TTGAGAAACCTAATATTGAGCAGGGTGTGCTGAACTTTGTGCAGTACAAGTTTAGTCACCTGGCTCCCCG
GGAGCGGCAGACGATGTTCGAGCTCTCAGATGTTCTTGCTCTGCCTTTAAACTACTGGGAGCTTGAGACA
CCTGCCCAGTTTCGGCAGAGGTCTCAGGCTGAGGACGTGGCTACCTACAAGGTCAATTACACCAGATGGC
TCTGTTACTGCCACGTGCCCCAGAGCTGTGATAGCCTCCCCCGCTACGAAACCACTCATGTCTTTGGGCG
AAGCCTTCTCCGGTCCATTTTCACCGTTACCCGCCGGCAGCTGCTGGAAAAGTTCCGAGTGGAGAAGGAC
AAATTGGTGCCCGAGAAGAGGACCCTCATCCTCACTCACTTCCCCAAATTCCTGTCCATGCTGGAGGAGG
AGATCTATGGGGCAAACTCTCCAATCTGGGAGTCAGGCTTCACCATGCCACCCTCAGAGGGGACACAGCT
GGTTCCCCGGCCAGGTTCAGTCAGTGCAGCGGTTGTTCCCAGCACCCCCATCTTCAGCCCCAGCATGGGT
GGGGGCAGCAACAGCTCCCTGAGTCTGGATTCTGCAGGGGCCGAGCCTATGCCAGGCGAGAAGAGGACGC
TCCCAGAGAACCTGACCCTGGAGGATGCCAAGCGGCTCCGTGTGATGGGTGACATCCCCATGGAGCTGGT
CAATGAGGTCATGCTGACCATCACTGACCCTGCTGCCATGCTGGGGCCTGAGACGAGCCTGCTTTCGGCC
AATGCGGCCCGGGATGAGACAGCCCGCCTGGAGGAGCGCCGCGGCATCATCGAGTTCCATGTCATCGGCA
ACTCACTGACGCCCAAGGCCAACCGGCGGGTGTTGCTGTGGCTCGTGGGGCTGCAGAATGTCTTTTCCCA
CCAGCTGCCGCGCATGCCTAAGGAGTATATCGCCCGCCTCGTCTTTGACCCGAAGCACAAGACTCTGGCC
TTGATCAAGGATGGGCGGGTCATCGGTGGCATCTGCTTCCGCATGTTTCCCACCCAGGGCTTCACGGAGA
TTGTCTTCTGTGCTGTCACCTCGAATGAGCAGGTCAAGGGTTATGGGACCCACCTGATGAACCACCTGAA
GGAGTATCACATCAAGCACAACATTCTCTACTTCCTCACCTACGCCGACGAGTACGCCATCGGCTACTTC
AAGCAGGGGCATTTCTCCAAGGACATCAAGGTGCCCAAGAGCCGCTACCTGGGCTACATCAAGGACTACG
AGGGAGCGACGCTGATGGATGTGAGCATGAATCCCCGCATCCCCTACACGGAGCTGTCCCACATCATCAA
GAAGCAGAAAGAGATCATCAAGAAGCTGATTGAGCGCAAACAGGCCCAGATCCGCAAGGTCTACCCGGGG
CTCAGCTGCTTCAAGGAGGGCGTGAGGCAGATCCCTGTGGAGAGCGTTCCTGGCATTCGAGAGACAGGCT
GGAAGCCATTGGGGAAGGAGAAGGGGAAGGAGCTGAAGGACCCCGACCAGCTCTACACAACCCTCAAAAA
CCTGCTGGCCCAAATCAAGTCTCACCCCAGTGCCTGGCCCTTCATGGAGCCTGTGAAGAAGTCGGAGGCC
CCTGACTACTACGAGGTCATCCGCTTCCCCATTGACCTGAAGACCATGACTGAGCGGCTGCGAAGCCGCT
ACTACGTGACCCGGAAGCTCTTTGTGGCCGACCTGCAGCGGGTCATCGCCAACTGTCGCGAGTACAACCC
CCCGGACAGCGAGTACTGCCGCTGTGCCAGCGCCCTGGAGAAGTTCTTCTACTTCAAGCTCAAGGAGGGA
GGCCTCATTGACAAGTAGGCCCATCTTTGGGCCGCAGCCCTGACCTGGAATGTCTCCACCTCGGATTCTG
ATCTGATCCTTAGGGGGTGCCCTGGCCCCACGGACCCGACTCAGCTTGAGACACTCCAGCCAAGGGTCCT
CCGGACCCGATCCTGCAGCTCTTTCTGGACCTTCAGGCACCCCCAAGCGTGCAGCTCTGTCCCAGCCTTC
ACTGTGTGTGAGAGGTCTCCTGGGTTGGGGCCCAGCCCCTCTAGAGTAGCTGGTGGCCAGAATGAAACCT
TGCCCAGCCGTGGTGGCCCCCAGGCCTGGTCCCCAAGAGCTTTGGAGGCTTGGATTCCTGGGACCTACCC
AGGTGGCTGTTTCCCGAGGACCAGAACTGCTCATTTTAGCTTGAGTGATGGCTTCAGGGGTTGGAAAGTT
CAGCCCAACTGAAGGGGGCCATGCCTTGTCCAGCACTGTTCTGTCAGTCTCCCCCAACGAAGATGAGGTA
TGGGGACCATTCATTCCCTGGCATTAATCCCTTAGAGGGAATAATAAAGCTTTTTATTTCTCTG
PCAF
SEQ ID NO:123
>gi | 6382075 | ref | NM_003884.2 | Homo sapiens p300/CBP-associated factor
(PCAF), mRNA
GGGGCCGCGTCGACGCGGAAAAGAGGCCGTGGGGGGCCTCCCAGCGCTGGCAGACACCGTGAGCTGGCAA
GCCGCCGGCACGCACACCTAGTCCGCAGTCCCGAGGAACATGTCACAGCCAGGGCGCGGAGCAGAGTCCA
CGGGCAGGAGAACCAAGGGAGGGCGTGTGCTGTGGCGGCGGCGGCAGCGGCAGCGGAGCCGCTAGTCCCC
TCCCTCCTGGGGGAGCAGCTGCCGCCGCTGCCGCCGCCGCCACCACCATCAGCGCGCGGGGCCCGGCCAG
AGCGAGCCGGGCGAGCGGCGCGCTAGGGGGAGGGCGGGGGCGGGGAGGGGGGGTGGGCGAAGGGGGCGGA
GGGCGTGGGGGGGAGGGTCTCGCTCTCCCGACTACCAGAGCCCGAGGGAGACCCTGGCGGCGGCGGCGCG
CCTGACACTCGGCGCCTCCTGCCGTGCTCCGGGGCGGCATGTCCGAGGCTGGCGGGGCCGGGCCGGGCGG
CTGCGGGGCAGGAGCCGGGGCAGGGGCCGGGCCCGGGGCGCTGCCCCCCGCAGCCTGCGGCGCTTCCGCC
GCGCCCCCGCAGGGCTCCCCCTGCGCCGCTGCCGCCGGGGGCTCGGGCGCCTGCGGTCCGGCGACGGCAG
TGGCTGCAGCGGGCACGGCCGAAGGACCGGGAGGCGGTGGCTCGGCCCGAATCGCCGTGAAGAAAGCGCA
ACTACGCTCCGCTCCGCGGGCCAAGAAACTGGAGAAACTCGGAGTGTACTCCGCCTGCAAGGCCGAGGAG
TCTTGTAAATGTAATGGCTGGAAAAACCCTAACCCCTCACCCACTCCCCCCAGAGCCGACCTGCAGCAAA
TAATTGTCAGTCTAACAGAATCCTGTCGGAGTTGTAGCCATGCCCTAGCTGCTCATGTTTCCCACCTGGA
GAATGTGTCAGAGGAACAAATGAACAGACTCCTGGGAATAGTATTGGATGTGGAATATCTCTTTACCTGT
GTCCACAAGGAAGAAGATGCAGATACCAAACAAGTTTATTTCTATGCTATTTAAGCTCTTGAGAAAGCTA
TTTTACAAAGAGGAAAACCTGTGGTTGAAGGCTCTTTGGAAAAGAAACCCCCATTTGAAAACCTAGACAT
TGAACAGGGTGTGAATAACTTTGTGCAGTACAAATTTAGTCACCTGCCAGCAAAAGAAAGGCAAACAATA
GTTGAGTTGGCAAAAATGTTCCTAAACCGCATCAACTATTGGCATCTGGAGGCACCATCTCAACGAAGAC
TGCGATCTCCCAATGATGATATTTCTGGATACAAAGAGAACTACACAAGGTGGCTGTGTTACTGCAACGT
GCCACAGTTCTGCGACAGTCTACCTCGGTACGAAACCACACAGGTGTTTGGGAGAACATTGCTTCGCTCG
GTCTTCACTGTTATGAGGCGACAACTCCTGGAACAAGCAAGACAGGAAAAAGATAAACTGCCTCTTGAAA
AACGAACTCTAATCCTCACTCATTTCCCAAAATTTCTGTCCATGCTAGAAGAAGAAGTATATAGTCAAAA
CTCTCCCATCTGGGATCAGGATTTTCTCTCAGCCTCTTCCAGTAACCAGCCAGCTAGGCATCCAACAGTT
ATCAATCCACCTCCTGTGGCTGGGACAATTTCATACAATTCAACCTCATCTTCCCTTGAGCAGCCAAACG
CAGGGAGCAGCAGTCCTGCCTGCAAAGCCTCTTCTGGACTTGAGGCAAAGGGAGGAGAAAAGAGGAAAAT
GACTGAATTCTCATGTTCTGGAGGAGCCAAGAAACCCCGAGTTATGGGGGATATTCCGATGGAATTAATC
AACGAGGTTATGTCTACCATCACGGACCCTGCAGCAATGCTTGGACCAGAGACCAATTTTCTGTCAGCAC
ACTCGGCCAGGGATGAGGCGGCAAGGTTGGAAGAGCGCAGGGGTGTAATTGAATTTCACGTGGTTGGCAA
TTCCCTCAACCAGAAACCAAACAAGAAGATCCTGATGTGGCTGGTTGGCCTACAGAACGTTTTCTCCCAC
CAGCTGCCCCGAATGCCAAAAGAATACATCACACGGCTCGTCTTTGACCCGAAACACAAAACCCTTGCTT
TAATTAAAGATGGCCGTGTTATTGGTGGTATCTGTTTCCGTATGTTCCCATCTCAAGGATTCACAGAGAT
TGTCTTCTGTGCTGTAACCTCAAATGAGCAAGTCAAGGGCTATGGAACACACCTGATGAATCATTTGAAA
GAATATCACATAAAGCATGACATCCTGAACTTCCTCACATATGCAGATGAATATGCAATTGGATACTTTA
AGAAACAGGGTTTCTCCAAAGAAATTAAAAATACCTAAAACCAAATATGTTGGCTATATCAAGGTTATGA
AGGAGCCACTTTAATGGGATGTGAGCTAAATCCACGGATCCCGTACACAGAATTTTCTGTCATCATTAAA
AGGAGCCACTTTAATGGGATGTGAGCTAAATCCACGGATCCCGTACACAGAATTTTCTGTCATCATTAAA
AAGCAGAAGGAGATAATTAAAAAACTGATTGAAAGAAAACAGGCACAAATTCGAAAAGTTTACCCTGGAC
TTTCATGTTTTAAAGATGGAGTTCGACAGATTCCTATAGAAAGCATTCCTGGAATTAGAGAGACAGGCTG
GAAACCGAGTGGAAAAGAGAAAAGTAAAGAGCCCAGAGACCCTGACCAGCTTTACAGCACGCTCAAGAGC
ATCCTCCAGCAGGTGAAGAGCCATCAAAGCGCTTGGCCCTTCATGGAACCTGTGAAGAGAACAGAAGCTC
CAGGATATTATGAAGTTATAAGGTCCCCATGGATCTGAAAACCATGAGTGAACGCCTCAAGAATAGGTAA
CTACGTGTCTAAGAAATTATTCATGGCAGACTTACAGCGAGTCTTTACAAATTGCAAAGAGTACAACGCC
GCTGAGAGTGAATACTACAAATGTGCCAATATCCTGGAGAAATTCTTCTCAGTAAAATTAAGGAACGCTG
GATTAATTGACAAGTGATTTTTTTTCCCCCTCTGCTTCTTAGAAACTCACCAAGCAGTGTGCCTAAAGCA
AGGT
MOZ
SEQ ID NO:124
>gi | 5803097 | ref | NM_006766.1 | Homo sapiens zinc finger protein 220
(ZNF220), mRNA
GGCACGAGGTTTGGGGCATCTCCGCGGTCCGGCCCGGGGCCCCGGGATCTCGGCTGTCCTTCCTCCCGGC
ATAAGATGCACATTTTTCTGCTCTGGAGCCGGGAATGAAATATTCTTGAGTTGTACAAAGTTTATGACGA
GACCCATGTGTGGTGCTTTAATGAGATTCATTGGGAAGTTGGAAGACATTTCAAACAACAGGTTGTTTTG
GTTTCTATAGTACATTGGGGTGGCATTCTGTTTTGTGAAGGAGGAAGGACTTAGGCCAGAAATTCACCAT
CGCATAGATCTGCAGCAGCCAGCTGCCAACCTGAGTGAGCTGTGCCGCAGTTGTGAGCACCCCTTGGCTG
ACCACGTATCCCACTTGGAGAATGTGTCAGAGGATGAGATAAACCGACTGCTGGGGATGGTGGTGGATGT
GGAGAATCTCTTCATGTCTGTTCACAAGGAAGAGGACACAGACACCAAGCAGGTCTATTTCTACCTCTTC
AAGCTACTGCGGAAATGCATCCTGCAGATGACCCGGCCTGTGGTGGAGGGGTCCCTGGGCAGCCCTCCAT
TTGAGAAACCTAATATTGAGCAGGGTGTGCTGAACTTTGTGCAGTACAAGTTTAGTCACCTGGCTCCCCG
GGAGCGGCAGACGATGTTCGAGCTCTCAGATGTTCTTGCTCTGCCTTTAAACTACTGGGAGCTTGAGACA
CCTGCCCAGTTTCGGCAGAGGTCTCAGGCTGAGGACGTGGCTACCTACAAGGTCAATTACACCAGATGGC
TCTGTTACTGCCACGTGCCCCAGAGCTGTGATAGCCTCCCCCGCTACGAAACCACTCATGTCTTTGGGCG
AAGCCTTCTCCGGTCCATTTTCACCGTTACCCGCCGGCAGCTGCTGGAAAAGTTCCGAGTGGAGAAGGAC
AAATTGGTGCCCGAGAAGAGGACCCTCATCCTCACTCACTTCCCCAAATTCCTGTCCATGCTGGAGGAGG
AGATCTATGGGGCAAACTCTCCAATCTGGGAGTCAGGCTTCACCATGCCACCCTCAGAGGGGACACAGCT
GGTTCCCCGGCCAGGTTCAGTCAGTGCAGCGGTTGTTCCCAGCACCCCCATCTTCAGCCCCAGCATGGGT
GGGGGCAGCAACAGCTCCCTGAGTCTGGATTCTGCAGGGGCCGAGCCTATGCCAGGCGAGAAGAGGACGC
TCCCAGAGAACCTGACCCTGGAGGATGCCAAGCGGCTCCGTGTGATGGGTGACATCCCCATGGAGCTGGT
CAATGAGGTCATGCTGACCATCACTGACCCTGCTGCCATGCTGGGGCCTGAGACGAGCCTGCTTTCGGCC
AATGCGGCCCGGGATGAGACAGCCCGCCTGGAGGAGCGCCGCGGCATCATCGAGTTCCATGTCATCGGCA
ACTCACTGACGCCCAAGGCCAACCGGCGGGTGTTGCTGTGGCTCGTGGGGCTGCAGAATGTCTTTTCCCA
CCAGCTGCCGCGCATGCCTAAGGAGTATATCGCCCGCCTCGTCTTTGACCCGAAGCACAAGACTCTGGCC
TTGATCAAGGATGGGCGGGTCATCGGTGGCATCTGCTTCCGCATGTTTCCCACCCAGGGCTTCACGGAGA
TTGTCTTCTGTGCTGTCACCTCGAATGAGCAGGTCAAGGGTTATGGGACCCACCTGATGAACCACCTGAA
GGAGTATCACATCAAGCACAACATTCTCTACTTCCTCACCTACGCCGACGAGTACGCCATCGGCTACTTC
AAGCAGGGGCATTTCTCCAAGGACATCAAGGTGCCCAAGAGCCGCTACCTGGGCTACATCAAGGACTACG
AGGGAGCGACGCTGATGGATGTGAGCATGAATCCCCGCATCCCCTACACGGAGCTGTCCCACATCATCAA
GAAGCAGAAAGAGATCATCAAGAAGCTGATTGAGCGCAAACAGGCCCAGATCCGCAAGGTCTACCCGGGG
CTCAGCTGCTTCAAGGAGGGCGTGAGGCAGATCCCTGTGGAGAGCGTTCCTGGCATTCGAGAGACAGGCT
GGAAGCCATTGGGGAAGGAGAAGGGGAAGGAGCTGAAGGACCCCGACCAGCTCTACACAACCCTCAAAAA
CCTGCTGGCCCAAATCAAGTCTCACCCCAGTGCCTGGCCCTTCATGGAGCCTGTGAAGAAGTCGGAGGCC
CCTGACTACTACGAGGTCATCCGCTTCCCCATTGACCTGAAGACCATGACTGAGCGGCTGCGAAGCCGCT
ACTACGTGACCCGGAAGCTCTTTGTGGCCGACCTGCAGCGGGTCATCGCCAACTGTCGCGAGTACAACCC
CCCGGACAGCGAGTACTGCCGCTGTGCCAGCGCCCTGGAGAAGTTCTTCTACTTCAAGCTCAAGGAGGGA
GGCCTCATTGACAAGTAGGCCCATCTTTGGGCCGCAGCCCTGACCTGGAATGTCTCCACCTCGGATTCTG
ATCTGATCCTTAGGGGGTGCCCTGGCCCCACGGACCCGACTCAGCTTGAGACACTCCAGCCAAGGGTCCT
CCGGACCCGATCCTGCAGCTCTTTCTGGACCTTCAGGCACCCCCAAGCGTGCAGCTCTGTCCCAGCCTTC
ACTGTGTGTGAGAGGTCTCCTGGGTTGGGGCCCAGCCCCTCTAGAGTAGCTGGTGGCCAGAATGAAACCT
TGCCCAGCCGTGGTGGCCCCCAGGCCTGGTCCCCAAGAGCTTTGGAGGCTTGGATTCCTGGGACCTACCC
AGGTGGCTGTTTCCCGAGGACCAGAACTGCTCATTTTAGCTTGAGTGATGGCTTCAGGGGTTGGAAAGTT
CAGCCCAACTGAAGGGGGCCATGCCTTGTCCAGCACTGTTCTGTCAGTCTCCCCCAACGAAGATGAGGTA
AGAAACAGGGTTTCTCCAAAGAAATTAAAAATACCTAAAACCAAATATGTTGGCTATATCAAGGTTATGA
AGGAGCCACTTTAATGGGATGTGAGCTAAATCCACGGATCCCGTACACAGAATTTTCTGTCATCATTAAA
AGGAGCCACTTTAATGGGATGTGAGCTAAATCCACGGATCCCGTACACAGAATTTTCTGTCATCATTAAA
AAGCAGAAGGAGATAATTAAAAAACTGATTGAAAGAAAACAGGCACAAATTCGAAAAGTTTACCCTGGAC
TTTCATGTTTTAAAGATGGAGTTCGACAGATTCCTATAGAAAGCATTCCTGGAATTAGAGAGACAGGCTG
GAAACCGAGTGGAAAAGAGAAAAGTAAAGAGCCCAGAGACCCTGACCAGCTTTACAGCACGCTCAAGAGC
ATCCTCCAGCAGGTGAAGAGCCATCAAAGCGCTTGGCCCTTCATGGAACCTGTGAAGAGAACAGAAGCTC
CAGGATATTATGAAGTTATAAGGTCCCCATGGATCTGAAAACCATGAGTGAACGCCTCAAGAATAGGTAA
CTACGTGTCTAAGAAATTATTCATGGCAGACTTACAGCGAGTCTTTACAAATTGCAAAGAGTACAACGCC
GCTGAGAGTGAATACTACAAATGTGCCAATATCCTGGAGAAATTCTTCTCAGTAAAATTAAGGAACGCTG
GTCTTCACTGTTATGAGGCGACAACTCCTGGAACAAGCAAGACAGGAAAAAGATAAACTGCCTCTTGAAA
AACGAACTCTAATCCTCACTCATTTCCCAAAATTTCTGTCCATGCTAGAAGAAGAAGTATATAGTCAAAA
CTCTCCCATCTGGGATCAGGATTTTCTCTCAGCCTCTTCCAGTAACCAGCCAGCTAGGCATCCAACAGTT
ATCAATCCACCTCCTGTGGCTGGGACAATTTCATACAATTCAACCTCATCTTCCCTTGAGCAGCCAAACG
CAGGGAGCAGCAGTCCTGCCTGCAAAGCCTCTTCTGGACTTGAGGCAAAGGGAGGAGAAAAGAGGAAAAT
GACTGAATTCTCATGTTCTGGAGGAGCCAAGAAACCCCGAGTTATGGGGGATATTCCGATGGAATTAATC
AACGAGGTTATGTCTACCATCACGGACCCTGCAGCAATGCTTGGACCAGAGACCAATTTTCTGTCAGCAC
ACTCGGCCAGGGATGAGGCGGCAAGGTTGGAAGAGCGCAGGGGTGTAATTGAATTTCACGTGGTTGGCAA
TTCCCTCAACCAGAAACCAAACAAGAAGATCCTGATGTGGCTGGTTGGCCTACAGAACGTTTTCTCCCAC
CAGCTGCCCCGAATGCCAAAAGAATACATCACACGGCTCGTCTTTGACCCGAAACACAAAACCCTTGCTT
TAAGGATAAAGAGGAAACCGAGCTGGATTCCGAAGAGGAGCAGCCTTCCCATGACACGTCCGTGGTGTCA
GTCTTCACTGTTATGAGGCGACAACTCCTGGAACAAGCAAGACAGGAAAAAGATAAACTGCCTCTTGAAA
AACGAACTCTAATCCTCACTCATTTCCCAAAATTTCTGTCCATGCTAGAAGAAGAAGTATATAGTCAAAA
CTCTCCCATCTGGGATCAGGATTTTCTCTCAGCCTCTTCCAGTAACCAGCCAGCTAGGCATCCAACAGTT
ATCAATCCACCTCCTGTGGCTGGGACAATTTCATACAATTCAACCTCATCTTCCCTTGAGCAGCCAAACG
CAGGGAGCAGCAGTCCTGCCTGCAAAGCCTCTTCTGGACTTGAGGCAAAGGGAGGAGAAAAGAGGAAAAT
GACTGAATTCTCATGTTCTGGAGGAGCCAAGAAACCCCGAGTTATGGGGGATATTCCGATGGAATTAATC
AACGAGGTTATGTCTACCATCACGGACCCTGCAGCAATGCTTGGACCAGAGACCAATTTTCTGTCAGCAC
ACTCGGCCAGGGATGAGGCGGCAAGGTTGGAAGAGCGCAGGGGTGTAATTGAATTTCACGTGGTTGGCAA
TTCCCTCAACCAGAAACCAAACAAGAAGATCCTGATGTGGCTGGTTGGCCTACAGAACGTTTTCTCCCAC
CAGCTGCCCCGAATGCCAAAAGAATACATCACACGGCTCGTCTTTGACCCGAAACACAAAACCCTTGCTT
GTCTTCACTGTTATGAGGCGACAACTCCTGGAACAAGCAAGACAGGAAAAAGATAAACTGCCTCTTGAAA
AACGAACTCTAATCCTCACTCATTTCCCAAAATTTCTGTCCATGCTAGAAGAAGAAGTATATAGTCAAAA
CTCTCCCATCTGGGATCAGGATTTTCTCTCAGCCTCTTCCAGTAACCAGCCAGCTAGGCATCCAACAGTT
ATCAATCCACCTCCTGTGGCTGGGACAATTTCATACAATTCAACCTCATCTTCCCTTGAGCAGCCAAACG
CAGGGAGCAGCAGTCCTGCCTGCAAAGCCTCTTCTGGACTTGAGGCAAAGGGAGGAGAAAAGAGGAAAAT
GACTGAATTCTCATGTTCTGGAGGAGCCAAGAAACCCCGAGTTATGGGGGATATTCCGATGGAATTAATC
AACGAGGTTATGTCTACCATCACGGACCCTGCAGCAATGCTTGGACCAGAGACCAATTTTCTGTCAGCAC
ACTCGGCCAGGGATGAGGCGGCAAGGTTGGAAGAGCGCAGGGGTGTAATTGAATTTCACGTGGTTGGCAA
TTCCCTCAACCAGAAACCAAACAAGAAGATCCTGATGTGGCTGGTTGGCCTACAGAACGTTTTCTCCCAC
CAGCTGCCCCGAATGCCAAAAGAATACATCACACGGCTCGTCTTTGACCCGAAACACAAAACCCTTGCTT
GTCTTCACTGTTATGAGGCGACAACTCCTGGAACAAGCAAGACAGGAAAAAGATAAACTGCCTCTTGAAA
AACGAACTCTAATCCTCACTCATTTCCCAAAATTTCTGTCCATGCTAGAAGAAGAAGTATATAGTCAAAA
CTCTCCCATCTGGGATCAGGATTTTCTCTCAGCCTCTTCCAGTAACCAGCCAGCTAGGCATCCAACAGTT
ATCAATCCACCTCCTGTGGCTGGGACAATTTCATACAATTCAACCTCATCTTCCCTTGAGCAGCCAAACG
CAGGGAGCAGCAGTCCTGCCTGCAAAGCCTCTTCTGGACTTGAGGCAAAGGGAGGAGAAAAGAGGAAAAT
GACTGAATTCTCATGTTCTGGAGGAGCCAAGAAACCCCGAGTTATGGGGGATATTCCGATGGAATTAATC
AACGAGGTTATGTCTACCATCACGGACCCTGCAGCAATGCTTGGACCAGAGACCAATTTTCTGTCAGCAC
ACTCGGCCAGGGATGAGGCGGCAAGGTTGGAAGAGCGCAGGGGTGTAATTGAATTTCACGTGGTTGGCAA
TTCCCTCAACCAGAAACCAAACAAGAAGATCCTGATGTGGCTGGTTGGCCTACAGAACGTTTTCTCCCAC
CAGCTGCCCCGAATGCCAAAAGAATACATCACACGGCTCGTCTTTGACCCGAAACACAAAACCCTTGCTT
GTCTTCACTGTTATGAGGCGACAACTCCTGGAACAAGCAAGACAGGAAAAAGATAAACTGCCTCTTGAAA
AACGAACTCTAATCCTCACTCATTTCCCAAAATTTCTGTCCATGCTAGAAGAAGAAGTATATAGTCAAAA
CTCTCCCATCTGGGATCAGGATTTTCTCTCAGCCTCTTCCAGTAACCAGCCAGCTAGGCATCCAACAGTT
ATCAATCCACCTCCTGTGGCTGGGACAATTTCATACAATTCAACCTCATCTTCCCTTGAGCAGCCAAACG
CAGGGAGCAGCAGTCCTGCCTGCAAAGCCTCTTCTGGACTTGAGGCAAAGGGAGGAGAAAAGAGGAAAAT
GACTGAATTCTCATGTTCTGGAGGAGCCAAGAAACCCCGAGTTATGGGGGATATTCCGATGGAATTAATC
AACGAGGTTATGTCTACCATCACGGACCCTGCAGCAATGCTTGGACCAGAGACCAATTTTCTGTCAGCAC
ACTCGGCCAGGGATGAGGCGGCAAGGTTGGAAGAGCGCAGGGGTGTAATTGAATTTCACGTGGTTGGCAA
TTCCCTCAACCAGAAACCAAACAAGAAGATCCTGATGTGGCTGGTTGGCCTACAGAACGTTTTCTCCCAC
CAGCTGCCCCGAATGCCAAAAGAATACATCACACGGCTCGTCTTTGACCCGAAACACAAAACCCTTGCTT
ACTTGCTCCAGGGATAGGTAAAAAAAAAA
HBO1
SEQ ID NO:125
>gi | 5901961 |ref | NM_007067.1 | Homo sapiens histone acetyltransferase
(HBOA), mRNA
GCCGCTGCCCGAATCGGAACCGTCGGGCCGCAGCCGCCGGCAATGCCGCGAAGGAAGAGGAATGCAGGCA
GTAGTTAGATGGAACCGAAGATTCCGATTTTTCTACAGATCTCGAGCACACACAGACAGTTCAGAAAGTG
TGGCACATCCCGACGATCTGCTCGAGTCACCCGCTCCTCAGCCAGGCTAAGCCAGAGTTCTCAAGATTCC
AGTCCTGTTCGAATCTGCAGTCTTTTGGCACTGAGGAGCCTGCTTACTCTACCAGAAGAGTGACCCCGTA
GTCAGCAGCAGCCTACCCCAGTGACACCGAAAAAATACCCTCTTCGGCAGACTCGTTCATCTGGTTCAGA
AACTGAGCAAGTGGTTGATTTTTCAGATAGAGAAACTAAAAATACAGCTGATCATGATGAGTCACCGCCT
CGAACTCCAACTGGAAATGCGCCTTCTTCTGAGTCTGACATAGATATCTCCAGCCCCAATGTATCTCACG
ATGAGAGCATTGCCAAGGACATGTCCCTGAAGGACTCAGGCAGTGATCTCTCTCATAACCCCAAGCGCCG
TCGCTTCCATGAAAGCTACAACTCAATATGAAGTGTCCTACACCAGGCTGTAACTCTCTAGCCCCACCTT
ACAGGAAACATGAGAGACATTTCTCCATCTCAGGATGCCCACTGTATCATAACTCTCAGCTGACGAAAAT
GCAAGGTGAGAGCACAGAGCCGGGATAAGCAGATAGAAGAAAGGATGCTGTCTCCAGGCAAGATGACAAA
CAACAGGCATGCAACCAGGCACCAGGCACCAACGGAGAGGCAGCTTCGTATAAGGAAAAAGTGGCTGAAA
CTCAGGAAGAAAAAGAAATTCTGGACTGAGCAAAGAACAGAAAGAGAAATATATGGAACAAGACAGACCT
ATGGGAACACACGGGAACCTCTTTTAGAAACCTGACAAGCGAGTATGACTTGGATCTTTTCCGAAGAGCA
ACAAGCCCGGGCTTCAGAGGATTTGGAGAAGTTAAGGCTGCAAGGCCAAATCACAGAGGGAAGCAACATG
ATTAAAACAATTGCTTTTGGCCGCTATGAGCTTGATACCTGGTATCATTCTCCATATCCTGAAGAATATG
CACGGCTGGGACGTCTCTATATGTGTGAATTCTGTTTAAAATATATGAAGAGCCAAACGATACTCCGCCG
GCACATGGCCAAATGTGTGTGGAAACACCCACCTGGTGATGAGATATATCGCAAAGGTTCAATCTCTGTG
TTTGAAGTGGATGGCAAGAAAAACAAGATCTACTGCCAAACCTGTGCCTGTTGGCCAAACTTTTTCTGGG
ACCACAAGACATTATATTATGATGTGGAGCCCTTCCTGTTCTATGTTATGACAGAGGCGGACAACACTGG
CTGTCACCTGATTGGATATTTTTCTAAGGAAAAGAATTCATTCCTCAACTACAACGTCTCCTGTATCCTT
ACTATGCCTCAGTACATGAGACAGGGCTATGGCAAGATGCTTATTGATTTCAGTTATTTGCTTTCCAAAG
TCGAAGAAAAAGTTGGCTCCCCAGAACGTCCACTCTCAGATCTGGGGCTTATAAGCTATCGCAGTTACTG
GAAAGAAGTACTTCTCCGCTACCTGCATAATTTTCAAGGCAAAGAGATTTCTATCAAAGAAATCAGTCAG
GAGACGGCTGTGAATCCTGTGGACATTGTCAGCACTCTGCAAGCCCTTCAGATGCTCAAATACTGGAAGG
GAAAACACCTAGTTTTAAAGAGACAGGACCTGATTGATGAGTGGATAGCCAAAGAGGCCAAAAGGTCCAA
CTCCAATAAAACCATGGATCCCAGCTGCTTAAAATGGACCCCTCCCAAGGGCACTTAAAGTGACCTGTCA
TTCCGAGCCAGCGAACCCCACAGTAGGAATCCGTACCTAGGATCTGTCTGCATTTCTCTGTTGCTCAAGC
TTGTGATTGGCAAGTACAGTATCCTTTGGGAAGGCCATCCCCCTCAGGACTGTCAACGCTCCGACCTTTG
TGTACACTGCAGACGCTGGTTCTGAGGAACTGTTGTTTCGGCCTCAGTGAGGTTGCCTGGATGGGATGCG
TATTAGACTTGAGTGCAGGTCTCTCAGCACTGACCCAAGGAGTTCTGTTATGGTACTGTACCTGTCCAGT
CACTGGTTCTCTCCTCATGTCCTCTCGCCCCATGAGGTTGTGTTGTGCTTCTAAGCGTGGTACTAGTGCA
TTGCCACCTGGTCACCAGACCTCCAAATATGGCTGCCACCACCAGGACCTTTCCAGTTACTCCTTATATG
TGTGTTCTATGGAGGGGCAGGGAAAAGGTGGCACTTGTGAGTGTGTGTGGATTGGCAGGGGGTCCATTCA
TTCCGTATCTCAGCCCATGACTCAGCAAGGCAGAATGGCCACCCCTGCCAAAGTTTGCTTCTCTTTTCAA
CATGCCTCACCCTCCCTCTAGGATTAAAGTGCTTCTGCCCTTCCACGAACTCCTCCTCCATTTCCTTTTT
TGGGATTTGTCACCATCCTTCTATTCTCTGGTCTTCTATTTTTGGTGTTGTTCAAGTGAAGGAAGAGATG
TTCCCTCTAATTTCTCTCTAGCCCATTATAACCTGCTATCTTGGGCAACTTTTGATGTATGACATGTCAA
CCCTTCCCAACTTGGTCTCCTCCAACATGCTGTCTTCATGTGGAGCCCTCACCAAATCCCTGACTCCGCG
TCATTTGTGCCTTTCTCTTGTCATCTCTGTACACTACTTATATTCACTGTGGGTTGGGGGAGCTAATTTT
AAGCATGTTCAGTGGCAGCTCCCCTCCAGTTTCAGTGTCACTGTTAAAATTTATCAAAAAGCAACTTCAC
TAGGGGTTTTCTTAAGGGATAAAGGCTTTTACAGAAGCTAAACCCTTCCCCACATGTGGTAGAATGTAGC
TCTTCTATATCTACTCCTCAATAAAGCATGTTCTCAATAAAGCATGTTCTCTGCTCAAAAAAAAAAAAAA
AAAA
SRC Family
SRC-1
SEQ ID NO:126
>gi | 4507198 | ref | NM_003743.1 | Homo sapiens nuclear receptor
coactivator 1 (NCOA1), mRNA
ATATCATCGACAGGGAGCACAGTGGGCTTCTCCTCAAGATGACACTAATTCTGGAATGTCAATTCCCCCG
AGTAAATCCCTCGGTCAATCCTAGTATCTCTCCAGCTCATGGTGTGGCTCGTTCATCCACATTGCCACCA
TCCAACAGCAACATGGTATCCACCAGAATAACCGCCAGCAGAGCTCAGACCTTACATAGCAGCAGTCATA
GTAATTCTAGCAACAGCCAAGGAAGTTTCGGATGCTCACCCGGAAGTCAGATTGTAGCCAATGTTGCCTT
AAACAAAAGGACAGGCCAGTTCACAGAGCAGTAAACCCTCTTAAACCTCAATAATCCTCCTATGGAAGGT
ACAGGAATATCCTAGCACAGTTCATGTCTCCAAGGAGACAGGTTACTTCTGGATTGGCAAACAAGGCCCA
GGATGCCAAACAATTCCTTTCCTCCTAATATTTCGACATTAAGCTCTCCCGTTGGCATGACAAGTAGTGC
CTGTAATAATAATAACCGATCTTATTCAAACATCCAGTAACATCTTTACAGGGTATGAATGAAAGGACCC
AATAACTCCGTTGGCTTCTCTGCCAGTTCTCCAGTCCTCAGGCAGATGAGCTCACAGAATTCACCTAGCA
GATTAAATATACAACCAGCAAAAGCTGAGTCCAAAGATAACAAAGAGATTGCCTCAACTTTAAATGAAAT
GATTCAATCTGACAACAGCTCTAGTGATGGCAAACCTCTGGATTCAGGGCTTCTGCATAACAATGACAGA
CTTTCAGATGGAGACAGTAAATACTCTCAAACCAGTCACAAACTAGTGCAGCTTTTGACAACAACTGCCG
AACAGCAGTTACGGCATGCTGATATAGACACAAGCTGCAAAGATGTCCTGTCTTGCACAGGCACTTCCAA
CTCTGCCTCTGCTAACTCTTCAGGAGGTTCTTGTCCCTCTTCTCATAGCTCATTGACAGCACGGCATAAA
ATTCTACACCGGCTCTTACAGGAGGGTAGCCCCTCAGATATCACCACTTTGTCTGTCGAGCCTGATAAAA
AGGACAGTGCATCTACTTCTGTGTCAGTGACTGGACAGGTACAAGGAAACTCCAGTATAAAACTAGAACT
GGATGCTTCAAAGAAAAAAGAATCAAAAGACCATCAGCTCCTACGCTATCTTTTAGATAAAGATGAGAAA
GATTTAAGATCAACTCCAAACCTGAGCCTGGATGATGTAAAGGTGAAAGTGGAAAAGAAAGAACAGATGG
ATCCATGTAATACAAACCCAACCCCAATGACGAAGGCCACTCCTGAGGAAATAAAACTGGAGGCCCAGAG
CCAGTTTACAGCTGACCTTGACCAGTTTGATCAGTTACTGCCCACGCTGGAGAAGGCAGCACAGTTGCCA
GGCTTATGTGAGACAGACAGGATGGATGGTGCGGTCACCAGTGTAACCATCAAATCGGAGATCCTGCCAG
CTTCACTTCAGTCCGCCACTGCCAGACCCACTTCCAGGCTGAATAGATTACCTGAGCTGGAATTGGAAGC
AATTGATAACCAATTTGGACAACCAGGAACAGGCGATCAGATTCCATGGACAAATAATACAGTGACAGCT
ATAAATCAGAGTAAATCAGAAGACCAGTGTATTAGCTCACAATTAGATGAGATTCTCTGTCCACCCACAA
CAGTAGAAGGGAGAAATGATGAGAAGGCTCTTCTTGAACAGCTGGTATCCTTCCTTAGTGGCAAAGATGA
AACTGAGCTAGCTGAACTAGACAGAGCTCTGGGAATTGACAAACTTGTTCAGGGGCGTGGATTAGATGTA
TTATCAGAGAGATTTCCACCACAACAAGCAACGCCACCTTTGATCATGGAAGAAAGACCCAACCTTTATT
CCCAGCCTTACTCTTCTCCTTTTCCTACTGCCAATCTCCCTAGCCCTTTCCAAGGCATGGTCAGGCAAAA
ACCTTCACTGGGGACGATGCCTGTTCAAGTAACACCTCCCCGAGGTGCTTTTTCACCTGGCATGGGCATG
CAGCCCAGGCAAACTCTAAACAGACCTCCGGCTGCACCTAACCAGCTTCGACTTCAACTACAGCAGCGAT
TACAGGGACAACAGCAGTTGATACACCAAAATCGGCAAGCTATCTTAAACCAGTTTGCAGCAACTGCTCC
TGTTGGCATCAATATGAGATCAGGCATGCAACAGCAAATTACACCTCAGCCACCCCTGAATGCTCAAATG
TTGGCACAACGTCAGCGGGAACTGTACAGTCAACAGCACCGACAGAGGCAGCTAATACAGCAGCAAAGAG
CCATGCTTATGAGGCAGCAAAGCTTTGGGAACAACCTCCCTCCCTCATCTGGACTACCAGTTCAAACGGG
GAACCCCCGTCTTCCTCAGGGTGCTCCACAGCAATTCCCCTATCCACCAAACTATGGTACAAATCCAGGA
ACCCCACCTGCTTCTACCAGCCCGTTTTCACAACTAGCAGCAAATCCTGAAGCATCCTTGGCCAACCGCA
ACAGCATGGTGAGCAGAGGCATGACAGGAAACATAGGAGGACAGTTTGGCACTGGAATCAATCCTCAGAT
GCAGCAGAATGTCTTCCAGTATCCAGGAGCAGGAATGGTTCCCCAAGGTGAGGCCAACTTTGCTCCATCT
CTAAGCCCTGGGAGCTCCATGGTGCCGATGCCAATCCCTCCTCCTCAGAGTTCTCTGCTCCAGCAAACTC
CACCTGCCTCCGGGTATCAGTCACCAGACATGAAGGCCTGGCAGCAAGGAGCGATAGGAAACAACAATGT
GTTCAGTCAAGCTGTCCAGAACCAGCCCACGCCTGCACAGCCAGGAGTATACAACAACATGAGCATCACC
GTTTCCATGGCAGGTGGAAATACGAATGTTCAGAACATGAACCCAATGATGGCCCAGATGCAGATGAGCT
CTTTGCAGATGCCAGGAATGAACACTGTGTGCCCTGAGCAGATAAATGATCCCGCACTGAGACACACAGG
CCTCTACTGCAACCAGCTCTCATCCACTGACCTTCTCAAAACAGAAGCAGATGGAACCCAGCAGGTGCAA
CAGGTTCAGGTGTTTGCTGACGTCCAGTGTACAGTGAATCTGGTAGGCGGGGACCCTTACCTGAACCAGC
GTGGTCCACTGGGAACTCAAAAGCCCACGTCAGGACCACAGACCCCCCAGGCCCAGCAGAAGAGCCTCCT
TCAGCAGCTACTGACTGAATAACCACTTTTAAAGGAATGTGAAATTTAAATAATAGACATACAGAGATAT
ACAAATATATTATATATTTTTCTGAGATTTTTGATATCTCAATCTGCAGCCATTCTTCAGGTCGTAGCAT
TTGGAGCAAAAAAAAAAAAAAAAAATCGATGTCGACTCGACGACACTACGGCGACAGCAACGG
PCIP
SEQ ID NO:127
>gi |5729725 | ref | NM_006534.1 | Homo sapiens nuclear receptor
coactivator 3 (NCOA3), mRNA
GTCGACGTGGCGGCCGGCGGCGGCTGCGGGCTGAGCGGCGAGTTTCCGATTTAAAGCTGAGCTGCGAGGA
AAATGGCGGCGGGAGGATCAAAATACTTGCTGGATGGTGGACTCAGAGACCAATAAAAATAAACTGCTTG
AACATCCTTTGACTGGTTAGCCAGTTGCTGATGTATATTCAAGATGAGTGGATTAGGAGAAAACTTGGAT
CCACTGGCCAGTGATTCACGAAAACGCAAATTGCCATGTGATACTCCAGGACAAGGTCTTACCTGCAGTG
GTGAAAAACGGAGACGGGAGCAGGAAAGTAAATATATTGAAGAATTGGCTGAGCTGATATCTGCCAATCT
TAGTGATATTGACAATTTCAATGTCAAACCAGATAAATGTGCGATTTTAAAGGAAACAGTAAGACAGATA
CGTCAAATAAAAGAGCAAGGAAAAACTATTTCCAATGATGATGATGTTCAAAAAGCCGATGTATCTTCTA
CAGGGCAGGGAGTTATTGATAAAGACTCCTTAGGACCGCTTTTACTTCAGGCATTGGATGGTTTCCTATT
TGTGGTGAATCGAGACGGAAACATTGTATTTGTATCAGAAAATGTCACACAATACCTGCAATATAAGCAA
GAGGACCTGGTTAACACAAGTGTTTACAATATCTTACATGAAGAAGACAGAAAGGATTTTCTTAAGAATT
CCATGCAGAAACCCCAGTATATCGATTCTCGTTGGCTGATGGAACTATAGTGACTGCACAGACAAAAAGC
AAACTCTTCCGAAATCCTGTAACAAATGATCGACATGGCTTTGTCTCAACCCACTTCCTTCAGAGAGAAC
AGAATGGATATAGACCAAACCCAAATCCTGTTGGACAAGGGATTAGACCACCTATGGCTGGATGCAACAG
TTCGGTAGGCGGCATGAGTATGTCGCCAAACCAAGGCTTACAGATGCCGAGCAGCAGGGCCTATGGCTTG
GCAGACCCTAGCACCACAGGGCAGATGAGTGGAGCTAGGTATGGGGGTTCCAGTAACATAGCTTCATTGA
CCCCTGGGCCAGGCATGCAATCACCATCTTCCTACCAGAACAACAACTATAGGCTCAACATGAGTAGCCC
CCCACATGGGAGTCCTGGTCTGCCCCAAACCAGCAGAATATCATGATTTCTCCATCGTAATCGTGGGAGT
CCAGATAGCCTCACATCAGTTTTCTCCTGTTGCAGGTGTGCACTCTCCCATGGCATCAATGAGACGCATA
CTGGGAACCACAGCTTTTCCAGCAGCTCTCTCAGTGCCCTGCAAGCCATCAGTGAAGGTGTGGGAAACGC
CCTTTTATGTACTCTGTCATCACCAGGCCCCATTGGACTATAACTCTCCCATAATGAATATTACCAACCA
AGTAAAGTAAGCAATCAGGATTCCAAGAGTCCTCTGGGCTTTTATTGCGACCAATCCAGTGGCAAGAGTT
CATGTGTCGTCAAATAGCAGAGATCACCTCAGTGACAAAGAAAGTAAGGAGAGCAGTGTACTGAGGGGGC
AGAGAATCAAAGGGGTCCTTTGGAAAGCAAAGGTCATAAAAATTACTGCAGTTACTTACCTGTTCTTTCT
GATGACCGGGGTCATTCCTCCTTGACCAACTCCCCCCTAGATTCAAGTTGTAAAGAATCTTCTGTTAGTG
TCACCAGCCCCTCTGGAGTCTCCTCCTTAACATGTGGAGGAGTATCCTCTACATCCAATATGCATAAGTC
ACTGTTACAAGAGAAGCACCGGATTTTGCACAAGTTGCTGCAGAATGGGAATTCACCAGCTGAGGTAGCC
AAGATTACTGCAGAGCCACTGGGAAGACACCAGCAGTATAACTTCTTGTGGGGACGGAAAGTAGTTGTCA
AGCAGGAGCAGCTAAGTCCTAAGAAGPAGGAGAATAATGCACTTCTTAGATACCTGCTGGACAGGGATGA
TCCTAGTGATGCACTCTCTAAAGAACTACAGCCCCAAGTGGAAGGAGTGGATAATAAAATAGAGTCAGTG
ACCAGCTCCACCATTCCTAGCTCAAGTCAAGAGAAAGACCCTAAAATTAAGACAGAGACAAGTGAAGAGG
GATCTGGAGACTTGGATAATCTAGATGCTATTCTTGGTGATCTGACTAGTTCTGACTTTTACAATAATTC
CATATCCTCAAATGGTAGTCATCGGGGACTAAGCAACAGGTGTTTCAAGGAACATAATTCTCTGGGTTTG
AAGTTCACAGTCTGTGCAGTCTATTCGTCCTCCATATAACCGAAGCAGTGTCTCTGGATAGCCCTTGTTT
CTGTTGGCTCAAGTCCTCCAGTAAAAAAATATCAGTGCTTTCCCCATGTTACCAAAGCAACCCATGTTGG
TGGGAATCCAAGAATGATGGATAGTCAGGAAAAATTATGGCTAAGTATGGGAGACTGGGGCTTACCAAAC
TCAAAGGCCGGCAGAATGGAACCTATGAATTCAAACTCCATGGGAAGACCAGGAGGAGATTATAATACTT
CTTTACCCAGACCTGCACTGGGTGGCTCTATTCCCACATTGCCTCTTCGGTCTAATAGCATACCAGGTGC
GAGACCAGTATTGCAACAGCAGCAGCAGATGCTTCAAATGAGGCCTGGTGAAATCCCCATGGGAATGGGG
AACAAGTTTCTCATGGCACTCAAAATAGGCCTCTTCTTAGGAATTCCCTGGATGATCTTGTTGGGCCACC
ACATCATGAGACCCCGGACAAACACCCCCAAGCAACTTAGAATGCAGCTTCAGCAGAGGCTGCAGGGCCA
GCAGTTTTTGAATCAGAGCCGACAGGCACTTGAATTGAAAATGGAAAACCCTACTGCTGGTGGTCGTGCG
GTGATGAGGCCTATGATGCAGCCCCAGCAGGGTTTTCTTAATGCTCAAATGGTCGCCCAACGCAGCAGAG
AGCTGCTAAGTCATCACTTCCGACAACAGAGGGTGGCTATGATGATGCAGCAGCAGCAACAGCAGCAGCA
GCAGCAGCAGCAGCAGCAACAGCAACAGCAACAGCAACAGCAGCAACAGCAGCAAACCCAGGCCTTCAGC
CCACCTCCTAATGTGACTGCTTCCCCCAGCATGGATGGGCTTTTGGCAGGACCCACAATGCCACAAGCTC
CTCCGCAACAGTTTCCATATCAACCAAATTATGGAATGGGACAACAACCAGATCCAGCCTTTGGTCGAGT
GTCTAGTCCTCCCAATGCAATGATGTCGTCTAGAATGGGTCCCTCCCAGAATCCCATGATGCAACACCCG
CAGGCTGCATCCATCTATCAGTCCTCAGAATTGAAGGGCTGGCCATCAGGAAATTTGGCCAGGAACAGCT
CCTTTTCCCAGCAGCAGTTTGCCCACCAGGGGAATCCTGCAGTGTATAGTATGGTGCACATGAATGGCAG
CAGTGGTCACATGGGACAGATGAACATGAACCCCATGCCCATGTCTGGCATGCCTATGGGTCCTGATCAG
AAATACTGCTGACATCTCTGCACCAGGACCTCTTAAGGAAACCACTGTACAAATGACACTGCAGTAGGAT
TATTGGGAAGGAATCATTGTTCCAGGCATCCATCTTGGAAGAAAGGACCAGCTTTGAGCTCCATCAAGGG
TATTTTTAAGTGATGTCATTTGAGCAGGACTGGATTTTAAAGCCGAAGGGCAATATCTACGTGTTTTCCC
CCTCCTTCTGCTGTGTATCATGGTGTTCAAAACAGAAATGTTTTTTGGCATTCCACCTCCTAGGGATATA
ATTCTGGAGACATGGAGTGTTACTGATCATAAAACTTTTGTGTCACTTTTTTCTGCCTTGCTAGCCAAAA
TCTCTTAAATACACGTAGGTGGGCCAGAGAACATTGGAAGAATCAAGAGAGATTAGAATATCTGGTTTCT
CTAGTTGCAGTATTGGACAAAGAGCATAGTCCCAGCCTTCAGGTGTAGTAGTTCTGTATTGACCCTTTGT
CCAGTGGAATTGGTGATTCTGAATTGTCCTTTACTAATGGTGTTGAGTTGCTCTGTCCCTATTATTTGCC
CTAGGCTTTCTCCTATGAAGGTTTTCATTTGCCATTCATGTCCTGTAATACTTCCACCTCCAGGAACTGT
CATGGATGTCCAAATGGCTTTGCAGAAGGAAATGAGATGACAGTATTTAATCAGCAGCAGTAGCAAACTT
TTCACATGCTAATGTGCAGCTGAGTGCACTTTATTTAAAAAGAATGGTAAATGCAATATTCTTGAGGCTC
TTGAGGGAATAGTGAAACACATTCCTGGTTTTTGCCTACACTTACGTGTTAGACAAGAACTATGATTTTT
TTTTTTAAAGTACTGGTGTCACCCTTTGCCTATATGGTAGAGCAATAATGCTTTTTAAAAATAAACTTCT
GAAAACCCAAGGCCAGGTACTGCATTCTGAATCAGAATCTCGCAGTGTTTCTGTGAATAGATTTTTTTGT
AAATATGACCTTTAAGATATTGTATTATGTAAAATATGTATATACCTTTTTTTGTAGGTCACAACAACTC
ATTTTTACAGAGTTTGTGAAGCTAAATATTTAACATGTTGATTTCAGTAAGCTGTGTGGTGAGGCTACCC
AGTGGAAGAGACATCCCTTGACTTTTGTGGCCTGGGGGAGGGGTAGTGCACCACAGCTTTTCCTTCCCAA
CCCCCCAGCCTTAGATGCCTCGCTCTTTTTAAAAATCCGGAGGACAAAATGCATCAACCCCAAATGCCAA
TAGATGTAGGCATTTTAATTTTTTAAAAATTCCTCTACCAGAACTAAGCACTTTGTTAATTTGGGGGGAA
AGAATAGATATGGGGAAATAAACTTTAAAAAAAAATCAGGAATTTAAAAAAACGAGCAATTTGAAGAGAA
TCTTTTGGATTTTAAGCAGTCCGAAATAATAGCAATTCATGGGCTGTGTGTGTGTGTGTATGTGTGTGTG
TGTGTGTGTATGTTTAATTATGTTACCTTTTCATCCCCTTTAGGAGCGTTTTCAGATTTTGGTTCGTAAG
ACCTGAATCCCATATTGAGATCTCGAGTAGATTCCTTGGTGTGGTTTCTGGTGTCTaCTCAGCTGTCCCC
TCATTCTACTATGTGATGCTTTCATTATGTCCCTGTGGATAGATAGTTAAAAGTATTATTTCTTAAGTAA
CTCAGTACCCAGAACAGCCAGTTTACTGTGATTCAGAGCCACAGTCTAACTGAGCACCTTTTAAACCCAC
TCCCTCTTCTGCCCCCTACCACTTTTCTGCTGTTGCCTCTCTTTGACACCTGTTTTAGTCAGTTGGGGAA
AAGGGAAAAATCAAGTTTAATTCCCTTTATCTGGGTTAATTCATTTGGTTCAAATAGTTGACGGAATTGG
GTTTCTGAATGTCTGTGAATTTCAGAGGTCTCTGCTAGCCTTGGTATCATTTTCTAGCAATAACTGAGAG
CCAGTTAATTTTAAGAATTTCACACATTTAGCCAAATCTTTCVTAGATGCTCTGAAGGTAAGATCATTTA
TATCTTTGATATGCTTACGAGTAAGTGAATCCTGATTATTTCCAGACCCACCACCAGAGTGGATCTTATT
TTCAAAGCAGTATAGACAATTATGAGTTTGCCTCTTTCCCCTACCAAGTTCAAATATATCTAAGAAAGAA
ATTGTAAATCCGAAACTTCCATTGTATGGCCTGTGCTTTTCAGATAGTATATCTCTCCTGTTTGGAGACA
AGAGGAAGAACCAGGTCAGTCTGTCTCTTTTTCAGCTCAATTGTATCTGACCCTTCTTTAAGTTATGTGT
GTGGGGAGAAATAGAATGGTGCTCTTATGTCGAC
GRIP1
SEQ ID NO:127
gi | 5729857 | ref | NM_006540.1 | Homo sapiens nuclear receptor coactivator
2 (NCOA2), mRNA
GGCGGCCGCAGCCTCGGCACAGCTTCGGCGGCGAAGGTCAGCGCCGACGGCAGCCGGCACCTGACAGGCG
TGACCGACCCGAGCCGATTTCTCTTGGATTTGGCTACACACTTATAGATCTTCTGCACTGTTTACAGGCA
CAGTTGCTGATATGTGTTCAAGATGAGTGGGATGGGAGAAAATACCTCTGACCCCTCCAGGGCAGAGACA
AGAAAGCGCAAGGAATGTCCTGACCAACTTGGACCCAGCCCCAAAAGGAACACTGAAAAACGTAATCGTG
AACAGGAAAATAAATATATAGAAGAACTTGCAGAGTGTGATTTTTGCAAATTTTAATGATATAGACAACT
TAACTTCAAACCTGACAAATGTGCAATCTTAAAAGAAACTGTGAAGCAAATTCGTCAGATCAAAGAACAA
GAGAAGCAGCAGCTGCCAACATAGATGAAGTGCAGAAGTCAGATGTATCCTCTACAGGGCAGGGTAGTCA
TCGACAGGATGCGCTGGGGCCTATGATGCTTGAGGCCCTTGATGGGTTCTTCTTTGTAGTGAACCATGGA
AGGCAACGTTGTGTTTGTGTCAGAGAATGTGACACAGTATCTAAGGTATAACCAAGAAGAGCTGATGAAC
AAAAGTGTATATAGCATCTTGCATGTTGGGGACCACACGGAATTTGTCAAAAACCTGCTGCCAAAGTCTA
TAGTAATGGGGGATCTTGGTCTGGCGACATCCGAGGCGGAGACAGCCATACCTTTCAATTGTCGGATGCT
GGTAAACCTTTACCTGATTCAGAAGAGGAGGGTCATGATAACCAAGGAAGCTCATCAGAAATATGAAACT
ATGCAGTGCTTCGCTGTCTCTCAACCAAAGTCCATCAAAGAAGAAGGAGAAGATTTGCAGTCCTGCTTGA
TTTGCGTGGCAAGAAGAGTTCCCATGAAGGAAAGACCAGTTCTTCCCTCATCAGAAAGTTTTACTACTCG
CCAGGATCTCCAAGGCAAGATCACGTCTCTGGATACCAGCACCATGAGAGCAGCCATGAAACCAGGCTGG
GAGGACCTGTAAGAAGGTGTATTCAGAAGTTCCATGCGCAGCATGAAGGAGAATCATGTGTCCTATGCTA
AGAGGCATCATCATGAAGTACTGAGACAAGGATTGGCATTCAGTCAAGTCTATCGTTTTTCCTTGTCTGA
TGGCACTCTTGTTGCTGCACAAACGAAGAGCAAACTCATCCGTTCTCAGACTACTAATGAACCTCAACTT
GTAATATCTTTACATATGCTTCACAGAGAGCAGAATGTGTGTGTGATGAATCCGGATCTGACTGGACAAA
CGATGGGGAAGCCACTGAATCCAATTAGCTCTAACAGCCCTGCCCATCAGGCCCTGTGCAGTGGGGAACC
AGGTCAGGACATGACCCTCAGTAGCAATATAAATTTTCCCATAAATGGCCAAAGGAACAAATGGGCAATG
CCCATGGGCAGGTTTGGTGGTTCTGGGGGATGAACCATGTGTCAGGCATGCAGCAACCAACTCCTCAAGG
GTAGTAAACTATGCACTCAAAATGAACAGCCCCTCACAAAGCAGCCCTGGCATGAATCCAGGACAGCCCA
CTCCATGCTATCACCAAGGCATCGCATGAGCCCTGGAGTGGCTGGCAGCCCTCGAATCCCACCCAGTCAG
TTTTCCCCTAGCAGGAAGCTTGCATTCCCCTGTGGGAGTTTGCAGCAGCAAGGAAATAGCCATAGTTATA
CCAACAGCACCCTCAATGCACTTCAGGCCCTCAGCGAGGGGCACGGGGTCTCATTAGGGTCATCGTTGGC
TTCACCAGACCTAAAATGGGCAATTTGCAAIAACTCCCCAGTTAATATGAATCCTCCCCCACTCAGCAAG
ATGGGAAAGCTGTGGACTCCAGACATGTTTTGGACTATATGGGGAGCCCTCTGAAGGTACAACTGGACAG
CATCTGGAGAATGGGCACCGCAGAGTTCGGCTGTGAGAGTCACCTGTGCTACTACCACCAGTGCCATGAA
CCGGCCAGTCCAAGGAGGTATGATTCGGAACCCAGCAGCCAGCATCCCCATGAGGCCCAGCAGCCAGCCT
GGCCAAAGACAGACGCTTCAGTCTCAGGTCATGAATATAGGGCCATCTGATTAGAGATGAACATGGAGGG
GACCTCAGTATAGCCAACAACAAGCTCCTCCAAATCAGACTGCCCCATGGCCTGAAAGCATCCTGCCTAT
AGACCAGGCGTCTTTTGCCAGCCAAAACAGGCAGCCATTTGGCAGTTCTCCAGATGACTTGCTATGTCCA
CATCCTGCAGCTGAGTCTCCGATAGATGAGGGAGCTCTCCTGGACCAGCTGTATCTGGCCTTGCGGAATT
TTGATGGCCTGGAGGAGATTGATAGAGCCTTAGGAATACCCGAACTGGTCAGCCAGAGCCAAGCAGTAGA
TCCAGAACAGTTCTCAAGTCAGGATTCCAACATCATGCTGGAGCAGAAGGCGCCCGTTTTCCCACAGCAG
TATGCATCTCAGGCACAAATGGCCCAGGGTAGCTATTCTCCCATGCAAGATCCAAACTTTCACACCATGG
GACAGCGGCCTAGTTATGCCACAAGCTCCGTATGCAGCCCAGACCGGGCCTCAGGCCAGGGCCTAGTGCA
GAACCAGCCAAATCAACTAAGACTTCAACTTCAGCATCGCCTCCAAGCACAGCAGAATCGCCAGCCACTT
ATGAATCAAATCAGCAATGTTTCCAAGATGTGGTCACTGACTCTGAGGCCTGGAGTACACACAGGACCTA
TTAATGCACAGATGCTGGCCCAGAGACAGAGGGAAATCCTGAACCAGCATCTTCGACAGAGACAAATGCA
TCAGCAACAGCAAGTTCAGCAACGAACTTTGATGAATGAGAGGACAAGGGTTGAATATGACACCAAGCAA
GTGGCTCCTAGTGGTATGCCAGCAACTATGAGCAACCCTCGGATTCCCCAGGCAATGCACAGCAGTTTTC
CATTTCCTCCAAACTACGGAATAAGTCAGCAACCTGATCCAGGCTTTACTGGGGCTACGACTCCCCAGAG
CCCACTTATGTCACCCCGAATGGCACATACACAGAGTCCCATGATGCAACAGTCTCAGGCCAACCCAGCC
TATCAGGCCCCCTCCCACATAAATGGATGGGCGCAGGGGAACATGGGCGGAAACAGCATGTTTTCCCAGC
AGTCCCCACCCACTTTGGGCAGCAAGCAAACACCAGCATGTACAGTAACAACATGAACATCAATGTGTCA
CATGGCGACCAACACAGGTGGCATGAGCAGCATGAACCAGATGACAGGACAGATCAGCATGACCTCAGTG
ACCTCCGTGCCTACCTCAGGGCTGTCCTCCATGGCTCCCGAGCAGGTTAATGATCCTGCTCTGAGGGGAG
GCAACCTGTTCCCAAACCAGCTGCCTGGAATGGATATGATTAAGCAGGAGGGAGACACAACACGGAAATA
TTGCTGACACTGCTGAAGCCAGTTGCTTCTTCAGCTGACCGGGCTCACTTGCTCAAAACACTTCCAAGTA
GGAGAGCTGTGTCTATTTGTTTCAACCCAACTGACCTGCCAGCCGGTTCTGCTAGAGCAGACAGGCCTGG
CCCTGGTTCCCAGGGTGGCGTCCACTCGGCTGTGGCAGGAGGAGCTGCCTCTTCTCTTGACAGTCTGAAG
CTCGCATCCAGACAGTCGCTCAGTCTGTTCCCTGCATTCACCTTAGTGCAACTTAGATCTCTCCTCCCCA
AGTAAATGTTGACAGGCCAATTTCATACCCATGTCAGATTGAATGTATTTAAATGTATGTATTTAAGGAG
AACCATGCTCTTGTTCTGTTCCTGTTCGGTTCCAGACACTGGTTTCTTGCTTTGTTTTCCCTGGCTAACA
GTCTAGTGCCAAAGATTAAGATTTTATCTGGGGGAAAGAAAAGAATTTTTTAAAAAATTAACTAAAGATA
GTTTTAAGCTAAAGCCTGAATTTGGGATGGAAGCAGGACAGACACCGTGGACAGCGCTGTATTTACAGAC
ACACCCAGTGCGTGAAGACCAACAAAGTCACAGTCGTATCTCTAGAAGCTCTAAAGACCATGTTGGAAAA
GAGTCTCCAGTTACTGAACAGATGAAAAGGAGCCTGTGAGAGGGCTGTTAACATTAGCAAATATTTTTTC
CTTGTTTTTTCTTTGTTAAAACCAAACTGGTTCACCTGAATCATGAATTGAGAAGAAATAATTTTCATTT
CTAAATTAAGTCCCTTTTAGTTTGATCAGACAGCTTGAATCAGCATCTCTTCTTCCCTGTAGCCTAGACT
CDTTCCCTTCCCCTCTCTCATTCCCCATACTCCCTATTTTCATTCCTTTTTTAAAAAATAATATAAGCTA
AGAAACCAGGTAAGCCCTTTTATTTCCTTAAAGTTTTGCCAGCCACTTACCAATTGCTAAGTATTGAATT
TCAGAAAAAAAAAATGCATTTACTGGCAAGGAGAAGAGCAAAGTTAAGGCTTGATACCAATCGAGCTAAG
GATACCTGCTTTGGAAGCATGTTTATTCTGTTCCCCAGCAACTCTGGCCTCCAAAATGGGAGAAACGCCA
GTGTGTTTAAATTGATAGCAGATATCACGACAGATTTAACCTCTGCCATGTGTTTTTTATTTTGTTTTTT
AGCAGTGCTGACTAAGCCGAAGTTTTGTAAGGTACATAAAATCCAATTTATATGTAAACAAGCAATAATT
TAAGTTGAGAACTTATGTGTTTTAATTGTATAATTTTTGTGAGGTAATATTGTGGAATTGACTCAAAACA
AATGAGGTACTTCAGTATTAAATTAGATATCTTCATAGCAATGTCTCCTAAAGGTGTTTTGTAAAGGATA
TCAATGCCTTGATTAGACCTAATTTGTAGACTTAAGACTTTTTATTTTCTAAACCTTGTGATTCTGCTTA
TAAGTCATTTATCTAATCTATATGATATGCAGCCGCTGTAGGAACCAATTCTTGATTTTTATATGTTTAT
ATTCTTTCTTAATGAACCTTAGAAAGACTACATGTTACTAAGCAGGCCACTTTTATGGTTGTTTTT
ATF-2
SEQ ID NO:128
>gi | 4503032 | ref | NM_001880.1 | Homo sapiens activating transcription
factor 2 (ATF2), mRNA
GAATTCTGTGATAAGTTATTCAACTTATGAAATTCAAGTTACATGTGAATTCTGCCAGGCAATACAAGGA
CCTGTGGAATATGAGTGATGACAAACCCTTTCTATGTACTGCGCCTGGATGTGGCCAGCGTTTTACCAAC
GAGGATCATTTGGCTGTCCATAAACATAAACATGAGATGACACTGAAATTTGGTCCAGCACGTAATGACA
GTGTCATTGTGGCTGATCAGACCCCAACACCAACAAGATTCTTGAAAAACTGTGAAGAAGTGGGTTTGTT
TAATGAGTTGGCGAGTCCATTTGAGAATGAATTCAAGAAAGCTTCAGAAGATGACATTAAAAAAATGCCT
CTAGATTTATCCCCTCTTGCAACACCTATCATAAGAAGCAAAATTGAGGAGCCTTCTGTTGTAGAAACAA
CTCACCAGGATAGTCCTTTACCTCACCCAGAGTCTACTACCAGTGATGAGAAGGAAGTACCATTGGCACA
AACTGCACAGCCCACATCAGCTATTGTTCGTCCAGCATCATTACAGGTTCCCAATGTGCTGCTTACAAGT
TCTGACTCAAGTGTAATTATTCAGCAGGCAGTACCTTCACCAACCTCAAGTACTGTAATCACCCAGGCAC
CATCCTCTAACAGGCCAATTGTCCCTGTACCAGGCCCATTTCCTCTTCTGTTACATCTTCCTAGTGGACA
CATCCTCTAACAGGCCAATTGTCCCTGTACCAGGCCCATTTCCTCTTCTGTTACATCTTCCTAGTGGACA
AACCATGCCTGTTGCTATTCTGCATCAATTACAAGTTCTAATGTGCATGTTCCAGCTGCAGTCCCACTCA
GTTCGACCAGTCACCATGGTGCCTAGTGTTCCAGGAATCCCAGGTCCTTCCTCTCCCCAACCAGTACAGT
CAGAAGCAAAAATGAGATTAAAAGCTGCTTTGACCCAGCAACATCCTCCAGTTACCAATGGTGATACTGT
GCCACATCCACTACAGAAACTCCGGCTTCTCCAGCTCACACAACTCCACAGACCCAAAGTACAAGTGGTC
GTCGGAGAAGAGCAGCTAACGAAGATCCTGATGAAAAAAGGAGAAAGTTTTTAGAGCGAATAGAGCAGCA
AGCTTCAAGATGCCGACAAAAAAGGAAAGTCTGGGTTCAGTCTTTAGAGAAGAAAGCTGAAGACTTGAGT
TCATTAAATGGTCAGCTGCAGAGTGAAGTCACCCTGCTGAGAAATGAAGTGGCACAGCTGAAACAGCTTC
TTCTGGCTCATAAAGATTGCCCTGTAACCGCCATGCAGAAGAAATCTGGCTATCATACTGCTGATAAAGA
TGATAGTTCAGAAGACATTCAGTGCCGAGTAGTCCACATACAGGAAGCTATACAGCATAGTTCGGTCAGC
ACATCCAATGGAGTCAGTTCAACCTCCAAGGCAGAAGCTGTAGCCACTTCAGTCCTCACCCAATGGCGGA
ACCAGAGTACAGAGCCTGCTCTTTCACAGATCGTTATGGCTCCTTCCTCCCAGTCACAGCCCTCAGGAAG
TTGATTAAAAACCTGCAGTACAACAGTTTAGATACTCATTAGTGACTTCAAAGGGAAATCAAGGAAAGAC
CAGTTTCCATTTATGCGAAATCTGTGGTTGTAAATTT
GNAT related
HAT1
SEQ ID NO:129
>gi | 4504340 | ref | Nm_003642.1 | Homo sapiens histone acetyltransferase 1
(HAT1), mRNA
CGTCCTTCCTCAGCCGCGGGTGATCGTAGCTCGGAAATGGCGGGATTTGGTGCTATGGAGAAATTTTTGG
TAGAATATAAGAGTGCAGTGGAGAAGAAACTGGCAGAGTACAAATGTAACACCAACACAGCAATTGAACT
AAAATTAGTTCGTTTTCCTGAAGATCTTGAAAATGACATTAGAACTTTCTTTCCTGAGTATACCCATCAA
CTCTTTGGGGATGATGAAACTGCTTTTGGTTACAAGGGTCTAAAGATCCTGTTATACTATATTGCTGGTA
GCCTGTCAACAATGTTCCGTGTTGAATATGCATCTAAAGTTGATGAGAACTTTGACTGTGTAGAGGCAGA
TGATGTTGAGGGCAAAATTAGACAATCATTCCACCTGGATTTTGCACAAACACGAATGATTTCCTTTCTA
TTACTGGAAAAGGAAGTTGATTTCAAGCCATTCGGAACCTTACTTCATACCTACTCAGTTCTCAGTCCAA
CAGGAGGAGAAAACTTTACCTTTCAGATATATAAGGCTGACATGACATGTAGAGGCATTTCGAGAATATC
TGAAAGGCTTCAGACCTTTTTGATGTGGTTTATTGAAACTGCTAGCTTTATTGACGTGGATGATGAAAGA
TGGCACTACTTTCTAGTATTTGAGAAGTATAATAAGGATGGAGCTACGCTCTTTGCGACCGTAGGCTACA
TGACAGTCTATAATTACTATGTGTACCCAGACAAAACCCGGCCACGTGTAAGTCAGATGCTGATTTTGAC
TCCATTTCAAGGTCAAGGCCATGGTGCTCAACTTCTTGAAACAGTTCATAGATACTACACTGAATTTCTT
ACAGTTCTTGATATTACAGCGGAAGATCCATCCAAAAGCTATGTGAAATTACGAGACTTTGTGCTTGTGA
AGCTTTGTCAAGATTTGCCCTGTTTTCCCGGGAAAAATTAATGCAAGGATTCAATGAAGATATGGCGATA
AGAGGCACAACAGAAGTTCAAAATAAATAAGCAACACGCTAGAAGGGTTTATGAAATTCTTCGACTACTG
GTAACTGACATGAGTGATGCCGAACAATACAGAAGCTACAGACTGGATATTAAAAGAAGACTAATTAGCC
CATATAAGAAAAAGCAGAGAGATCTTGCTAAGATGAGAAAATGTCTCAGACCAGAAGAACTGACAAACCA
GATGAACCAAATAGAAATAAGCATGCAACATGAACAGCTGGAAGAGAGTTTTCAGGAACTAGTGGAAGAT
TACCGGCGTGTTATTGAACGACTTGCTCAAGAGTAAAGATTATACTGCTCTGTACAGGAAGCTTGCAAAT
TTTCTGTACAATGTGCTGTGAAAAATCTGATGACTTTAATTTTAAAACTTGTGACATTTTGCTTATACTG
AAAAGTTATCTATCTTTAGTTAGAATATTTTCTTTTGGAGAGATTGTATATTTTAAAATACTGTTTAGAG
TTATGAGCATATATTGCATTTAAAGAAAGATAAAGCTTCTGAAATACTACTGAAATTGCTTCCCTTCTTA
AACAGTATAATAAJATGCTTAGTTGTAT

TABLE 6

ATP-dependent Chromatin Remodelling.

SMARCA5
SEQ ID NO:130
>gi | 4507074 | ref | NM_003601.1 | Homo sapiens SWI/SNF related, matrix
associated, actin dependent regulator of chromatin, subfamily a,
member 5 (SMARCA5), mRNA
GCGGAAGAGCAGAACGTTTGGGAGTGTGCAGCTCCTGGGCCCGGCTCAGGCCCGTCGCGGAGGCGCGGCG
CAGGGGAGCGCTCGGGTGGGAGTCTCGCTCCTCCACCAGTTTATTGCGACGTAGCATCCAGGCCTAGGCC
TCCCCGTCCATCCCCGCCGGACTCGGGCCTCTGGCAGCAGCGGGTGACGCAGACGGAACATCATGTCGTC
CGCGGCCGAGCCTCCGCCACCCCCGCCTCCCGAGAGCGCGCCTTCCAAGCCCGCAGCCTCGATCGCCAGC
GGCGGGAGCAACAGCAGCAACAAAGGCGGCCCCGAAGGCGTCGCGGCGCAGGCGGTTGCGTCTGCGGCCA
GCGCTGGTCCCGCAGACGCCGAGATGGAGGAAATATTTGATGATGCGTCACCTGGAAAGCAAAAGGAAAT
CCAAGAACCAGATCCTACCTATGAAGAAAAAATGCAAACTGACCGGGCAAATAGATTCGAGTATTTATTA
AAGCAGACAGAACTTTTTGCACATTTCATTCAACCTGCTGCTCAGAAGACTCCAACTTCACCTTTGAAGA
TGAAACCAGGGCGCCCACGAATAAAAAAAGATGAGAAGCAGAACTTACTATCCGTTGGCGATTACCGACA
CCGTAGAACAGAGCAAGAGGAGGATGAAGAGCTATTAACAGAAAGCTCCAAAGCAACCAATGTTTGCACT
CGATTTGAAGACTCTCCATCGTATGTAAAATGGGGTAAACTGAGAGATTATCAGGTCCGAGGATTAAACT
GGCTCATTTCTTTGTATGAGAATGGCATCAATGGTATCCTTGCAGATGAAATGGGCCTAGGAAAGACTCT
TCAAACAATTTCTCTTCTTGGGTACATGAAACATTATAGAAACATTCCTGGGCCTCATATGGTTTTGGTT
CCTAAGTCTACATTACACAACTGGATGAGTGAATTCAAGAGATGGGTACCAACACTTAGATCTGTTTGTT
TGATAGGAGATAAAGAACAAAGAGCTGCTTTTGTCAGAGACGTTTTATTACCGGGAGAATGGGATGTATG
TGTAACATCTTATGAAATGCTTATTAAAGAGAAGTCTGTGTTCAAAAAATTTAATTGGAGATACTTAGTA
ATAGATGAAGCTCACAGGATCAAAAATGAAAAATCTAAGTTGTCAGAAATAGTGAGGGAATTCAAGACTA
CAAATAGACTATTATTAACTGGAACACCTCTTCAGAACAACTTGCATGAGCTGTGGTCACTTCTTAACTT
TCTGTTGCCAGATGTGTTTAATTCAGCAGATGACTTTGATTCCTGGTTTGATACAAACAACTGCCTTGGG
GATCAAAAACTAGTTGAGAGGCTTCATATGGTTTTGCGTCCATTCCTCCTTCGTCGAATTAAGGCTGATG
TTGAAAAGAGTTTGCCTCCAAAGAAGGAAGTAAAAATCTATGTGGGCCTCAGCAAAATGCAAAGGGAATG
GTATACTCGGATATTAATGAAGGATATAGATATACTCAACTCAGCAGGCAAGATGGACAAAATGAGGTTA
TTGAACATCCTAATGCAGTTGAGAAAATGTTGTAATCATCCATATCTCTTTGATGGAGCAGAACCTGGTC
CACCTTATACAACAGATATGCATCTAGTAACCAACAGTGGCAAAATGGTGGTTTTAGACAAGCTGCTCCC
TAAGTTAAAAGAACAAGGTTCACGAGTACTAATCTTCAGTCAAATGACAAGGGTATTGGACATTTTGGAA
GATTATTGCATGTGGAGAAATTATGAGTACTGCAGGTTGGATGGTCAGACACCCCATGATGAGAGACAAG
ACTCCATCAATGCATACAATGAACCAAACAGCACAAAGTTTGTTTTCATGTTAAGCACGCGTGCTGGTGG
TCTTGGCATCAATCTTGCGACTGCTGATGTAGTAATTTTGTATGATTCTGATTGGAATCCCCAAGTAGAT
CTTCAGGCTATGGACCGAGCACATAGAATTGGGCAGACTAAGACAGTCAGAGTGTTCCGCTTTATAACTG
ATAACACTGTAGAAGAAAGAATAGTAGAACGTGCTGAGATGAAACCTCAGACTGGATTCAATAGTCATCA
ACAAGGGAGGCTTGTGGATCAGAATCTGAACAAAATTGGGAAAGATGAAATGCTTCAAATGATTAGACAT
GGAGCAACACATGTGTTTGCTTCAAAGGAAAGTGAGATCACTGATGAAGATATCGATGGTATTTTGGAAA
GAGGTGCAAAGAAGACTGCAGAGATGAATGAAAAGCTCTCCAAGATGGGCGAAAGTTCACTTAGAAACTT
TACAATGGATACAGAGTCAAGTGTTTATAACTTCGAAGGAGAAGACTATAGAGAAAAACAAAAGATTGCA
TTCACAGAGTGGATTGAACCACCTAAACGAGAAAGAAAAGCCAACTATGCCGTTGATGCATATTTCAGGG
AAGCTCTTCGTGTTAGTGAACCTAAAGCACCCAAGGCTCCTCGACCTCCAAAACAACCCAATGTTCAGGA
TTTCCAGTTCTTTCCTCCACGTTTATTTGAATTACTGGAAAAAGAAATTCTGTTTTACAGAAAAACTATT
GGGTACAAGGTACCTCGAAATCCTGAGCTGCCTAACGCAGCACAGGCACAAAAAGAAGAACAGCTTAAAA
TTGATGAAGCTGAATCCCTTAATGATGAAGAGTTAGAGGAAAAAGAGAAGCTTCTAACACAGGGATTTAC
CAATTGGAATAAGAGAGATTTTAACCAGTTTATCAAAGCTAATGAGAAGTGGGGTCGTGATGATATTGAA
AATATAGCAAGAGAAGTAGAAGGCAAAACTCCAGAAGAAGTCATTGAATATTCAGCTGTGTTTTGGGAAA
GGTGCAACGAGCTCCAGGACATAGAGAAGATTATGGCTCAGATTGAATGGGGAGAGGCGAGAATTCAAAG
AAGAATAAGCATCAAGAAAGCACTTGACACAAAGATTGGACGGTACAAAGCACCTTTTCATCAGCTGAGA
ATATCATATCGTACTAACAAAGGAAAAAACTATACTGAAGAAGAAGATCGTTTTCTGATTTGTATGCTTC
ACAAACTTGGATTTGACAAAGATAATGTTTATGATGAATTGCGACAGTGTATTCGCAACTCTCCTCAGTT
CAGATTTGACTGGTTTCTTAAGTCCAGAACTGCAATGGAGCTCCAGAGGAGATGTAATACCTTAATTACT
TTGATTGAAAGAGAAAACATGGAACTAGAAGAAAAGGAGAAGGCAGAGAAAAAGAAACGAGGACCAAAGC
CTTCAACACAGAAACGTAAAATGGATGGCGCACCTGATGGTCGAGGAAGAAAAAAGAAGCTGAAACTATG
AATATGTTTTTGTTTCATAATCAGTAACTTTAAACCAGTAGTTCTTTAATTTACGGGTCTTCATAAGATG
TACTGTACAATGCTCAATTGTTATGTCATTTAAAGACATCAGGTTCATCTGTTTACTGAGCTAGAAACAT
AGTATGTAGTTTCACTTTTTTAAATGCAACAGCTGTGCTGAAATTTTTTTATCATTAACACTTGAAGTAA
TAAAATAGGCTTCATTTATTAAAAAAAAAAAAAAAA
SMARCA2
SEQ ID NO:131
>gi | 4507068 | ref | NM_003070.1 | Homo sapiens SWI/SNF related, matrix
associated, actin dependent regulator of chromatin, subfamily a,
member 2 (SMARCA2), mRNA
AAAAATTTTCTGTTACCAAATTTTACAACTTCTAATAAGACTACTATAACTTTATGTAAACTGATGAAGA
TGTGCTGATTAACATATTCTGTGATATGGTTTACAACTTTTAATCATAATTGTCCATGATTTTGGAATGC
TGTTATTTATCAGTAAATGTAAAATATTTGAGGCATTTAGCCATACACACACTAGAACTTTTTAAAACTT
TGTCCTATAGTGTAATTATAAACTGATGACTATTATCTTCATACATTGAGTCTTCATGCATCAATGAAAT
GAAAAATATAGGAGTAGATGTCCACGCCCACAGACCCTGGTGCGATGCCCCACCCAGGGCCTTCGCCGGG
GCCTGGGCCTTCCCCTGGGCCAATTCTTGGGCCTAGTCCAGGACCAGGACCATCCCCAGGTTCCGTCCAC
AGCTGATGGGGCCAAGTCCTGGACCTCCAAGTGTCTCCCATCCTATGCCGACCGATGGGGTCCACAGACT
TCCCACAGGAAGGCATGCATCAAATGCATAAGCCCATCGATGGTATACATGACAAGGGGATTGTAGAAGA
CATCCATTGTGGATCCATGAAGGGCACTGGTATGCGACCACCTCACCCAGGCATGGGCCCTCCCCAGAGT
CCAATGGATCAACACAGCCAAGGTTATATGTCACCACACCCATCTCCATTAGGAGCCCCAGAGCACGTCT
CCAGCCCTATGTCTGGAGGAGGCCCAACTCCACCTCAGATGCCACCAAGCCAGCCGGGGGCCCTCATCCC
AGGTGATCCGCAGGCCATGAGCCAGCCCAACAGAGGTCCCTCACCTTTCAGTCCTGTCCAGCTGCATCAG
CTTCGAGCTCAGATTTTAGCTTATAAAATGCTGGCCCGAGGCCAGCCCCTCCCCGAAACGCTGCAGCTTG
CAGTCCAGGGGAAAAGGACGTTGCCTGGCTTGCAGCAACAACAGCAGAAGCAACAGCAGCAGCAGCAGCA
GCAGCAGCAGCAGCAGCAGCAGCAACAGCAGCCGCAGCAGCAGCCGCCGCAACCACAGACGCAGCAACAA
ACAAACGGAGCAAGCGCCAGACTCTGAGAGAAGCTCGCATGACCGAGAAGCTGGAGAAGCAGCAGAAGAT
TGAGCAGGAGAGGAAACGCCGTCAGAAACACCAGGAATACCTGAACAGTATTTTGCAACATGCAAAAGAT
TTTAAGGAATATCATCGGTCTGTGGCCGGAAAGATCCAGAAGCTCTCCAAAGCAGTGGCAACTTGGCATG
CCAACACTGAAAGAGAGCAGAAGAAGGAGACAGAGCGGATTGAAAAGGAGAGAATGCGGCGACTGATGGC
TGAAGATGAGGAGAGTTATAGAAAACTGATTGATCAAAAGAAAGACAGGCGTTTAGCTTACCTTTTGCAG
CAGACCGATGAGTATGTAGCCAATCTGACCAATCTGGTTTGGGAGCACAAGCAAGCCCAGGCAGCCAAAG
AGAAGAAGAAGAGGAGGAGGAGGAAGAAGAAGGCTGAGGAGAATGCAGAGGGTGGGGAGTCTGCCOTGGG
ACCGGATGGAGAGCCCATAGATGAGAGCAGCCAGATGAGTGACCTCCCTGTCAAAGTGACTCACACAGAA
ACCGGCAAGGTTCTGTTCGGACCAGAAGCACCCAAAGCAAGTCAGCTGGACGCCTGGCTGGAAATGAATC
CTGGTTATGAAGTTGCCCCTAGATCTGACAGTGAAGAGAGTGATTCTGATTATGAGGAAGAGGATGAGGA
AGAAGAGTCCAGTAGGCAGGAAACCGAAGAGAAAATACTCCTGGATCCAAATAGCGAAGAAGTTTCTGAG
AAGGATGCTAAGCAGATCATTGAGACAGCTAAGCAAGACGTGGATGATGAATACAGCATGCAGTACAGTG
CCAGGGGCTCCCAGTCCTACTACACCGTGGCTCATGCCATCTCGGAGAGGGTGGAGAAACAGTCTGCCCT
CCTAATTAATGGGACCCTAAAGCATTACCAGCTCCAGGGCCTGGAATGGATGGTTTCCCTGTATAATAAC
AACTTGAACGGAATCTTAGCCGATGAAATGGGGCTTGGAAAGACCATACAGACCATTGCACTCATCACTT
ATCTGATGGAGCACAAAAGACTCAATGGCCCCTATCTCATCATTGTTCCCCTTTCGACTCTATCTAACTG
GACATATGAATTTGACAAATGGGCTCCTTCTGTGGTGAAGATTTCTTACAAGGGTACTCCTGCCATGCGT
CGCTCCCTTGTCCCCCAGCTACGGAGTGGCAAATTCAATGTCCTCTTGACTACTTATGAGTATATTATAA
AAGACAAGCACATTCTTGCAAAGATTCGGTGGAAATACATGATAGTGGACGAAGGCCACCGAATGAAGAA
TCACCACTGCAAGCTGACTCAGGTCTTGAACACTCACTATGTGGCCCCCAGAAGGATCCTCTTGACTGGG
ACCCCGCTGCAGAATAAGCTCCCTGAACTCTGGGCCCTCCTCAACTTCCTCCTCCCAACAATTTTTAAGA
GCTGCAGCACATTTGAACAATGGTTCAATGCTCCATTTGCCATGACTGGTGAAAGGGTGGACTTAAATGA
AGAAGAAACTATATTGATCATCAGGCGTCTACATAAGGTGTTAAGACCATTTTTACTAAGGAGACTGAAG
AAAGAAGTTGAATCCCAGCTTCCCGAAAAAGTGGAATATGTGATCAAGTGTGACATGTCAGCTCTGCAGA
AGATTCTGTATCGCCATATGCAAGCCAAGGGGATCCTTCTCACAGATGGTTCTGAGAAAGATAAGAAGGG
CGCATCGGGCAGCAGAACGAGGTCCGGGTACTGAGGCTCTGTACCGTGAACAGCGTGGAGGAAAAGATCC
TCGCGGCCGCAAAATACAAGCTGAACGTGGATCAGAAAGTGATCCAGGCGGGCATGTTTGACCAAAAGTC
TTCAAGCCACGAGCGGAGGGCATTCCTGCAGGCCATCTTGGAGCATGAAGAGGAAAATGAGGAAGAAGAT
GAAGTACCGGACGATGAGACTCTGAACCAAATGATTGCTCGACGAGAAGAAGAATTTGACCTTTTTATGC
GGATGGACATGGACCGGCGGAGGGAAGATGCCCGGAACCCGAAACGGAAGCCCCGTTTAATGGAGGAGGA
TGAGCTGCCCTCCTGGATCATTAAGGATGACGCTGAAGTAGAAAGGCTCACCTGTGAAGAAGAGGAGGAG
AAAATATTTGGGAGGGGGTCCCGCCAGCGCOGTGACGTGGACTACAGTGACGCCCTCACGGAGAAGCAGT
GGCTAAGGGCCATCGAAGACGGCAATTTGGAGGAAATGGAAGAGGAAGTACGGCTTAAGAAGCGAAAAAG
ACGAAGAAATGTGGATAAAGATCCTGCAAAAGAAGATGTGGAAAAAGCTAAGAAGAGAAGAGGCCGCCCT
CCCGCTGAGAAACTGTCACCAAATCCCCCCAAACTGACAAAGCAGATGAACGCTATCATCGATACTGTGA
TAAACTACAAAGATAGTTCAGGGCGACAGCTCAGTGAAGTCTTCATTCAGTTACCTTCAAGGAAAGAATT
ACCAGAATACTATGAATTAATTAGGAAGCCAGTGGATTTCAAAAAAATAAAGGAAAGGATTCGTAATCAT
AAGTACCGGAGCCTAGGCGACCTGGAGAAGGATGTCATGCTTCTCTGTCACAACGCTCAGACGTTCAACC
TGGAGGGATCCCAGATCTATGAAGACTCCATCGTCTTACAGTCAGTGTTTAAGAGTGCCCGGCAGAAAAT
TGCCAAAGAGGAAGAGAGTGAGGATGAAAGCAATGAAGAGGAGGAAGAGGAAGATGAAGAAGAGTCAGAG
TCCGAGGCAAAATCAGTCAAGGTGAAAATTAAGCTCAATAAAAAAGATGACAAAGGCCGGGACAAAGGGA
AAGGCAAGAAAAGGCCAAATCGAGGAAAAGCCAAACCTGTAGTGAGCGATTTTGACAGCGATGAGGAGCA
AGGATGAACGTGAACGTCAGAAGGAAGTGGGACGGATGATGAGTGATCAGTATGGACCTTTTTCCTTGGT
AGAACTGAATTCCTTCCTCCCCTGTCTCATTTCTACCCAGTGAGTTCATTTGTCATATAGGCACTGGGTT
GTTTCTATATCATCATCGTCTATAAACTAGCTTTAGGATAGTGCCAGACAAACATATGATATCATGGTGT
AAAAAACACACACATACACAAATATTTGTGACCAAATGGGCCTCAAAGATTCAGATTGAAACAAACAAAA
AGCTTTT
SMARCA4
SEQ ID NO:132
>gi | 4507072 | ref | NM_003072.1 | Homo sapiens SWI/SNF related, matrix
associated, actin dependent regulator of chromatin, subfamily a,
member 4 (SMARCA4), mRNA
TTCCGGCGGGGGAGGCGCCGGGAAGTCGATGGCGCCGGCGGCTCCTGCAGGAGGCCACTGTCTGCAGCTC
CCGTGAAGATGTCCACTCCAGACCCACCCCTGGGCGGAACTCCTCGGCCAGGTCCTTCCCCGGGCCCTGG
CCCTTCCCCTGGAGCCATGCTGGGCCCTAGCCGGGTCCCTCGCCGGGCTCCGCCCCACAGCATGATGGGG
CCCAGCCCAGGGCCGCCCTCAGCAGGACACCCCATCCCCACCCAGGGGCCTGGAGGGTACCCTCAGGACA
ACATGCACCAGATGCACAAGCCCATGGAGTCCATGCATGAGAAGGGCATGTCGGACGACCCGCGCTACAA
CCAGATGAAAGGAATGGGGATGCGGTCAGGGGGCCATGCTGGGATGGGGCCCCCGCCCAGCCCCATGGAC
CAGCACTCCCAAGGTTACCCCTCGCCCCTGGGTGGCTCGAGCATGCCTCTAGTCCAGTTCCAGCCCAGTG
GCCCGTCTTCGGGGCCCCAGATGTCTTCCGGGCCAGGAGGTGCCCCGCTGGATGGTGCTGACCCCCAGGC
CTTGGGGCAGCAGAACCGGGGCCCAACCCCATTTAACCAGAACCAGCTGCACCAGCTCAGAGCTCAGATC
ATGGCCTACAAGATGCTGGCCAGGGGGCAGCCCCTCCCCGACCACCTGCAGATGGCGGTGCAGGGCAAGC
GGCCGATGCCCGGGATGCAGCAGCAGATGCCAACGCTACCTCCACCCTCGGTGTCCGCAACAGGACCCGG
CCCTGGCCCTGGCCCTGGCCCCGGCCCGGGTCCCGGCCCGGCACCTCCAAATTACAGCAGGCCTCATGGT
ATGGGAGGGCCCAACATGCCTCCCCCAGGACCCTCGGGCGTGCCCCCCGGGATGCCAGGCCAGCCTCCTG
GAGGGCCTCCCAAGCCCTGGCCTGAAGGACCCATGGCGAATGCTGCTGCCCCCACGAGCACCCCTCAGAA
GCGATTCCCCCGCAGCCAACGGGCCGCCCTTCCCCCGCGCCCCCTGCCGTCCCACCCGCCGCCTCGCCCC
GTGATGCCACCGCAGACCCAGTCCCCCGGGCAGCCGGCCCAGCCCGCGCCCATGGTGCCACTGCACCAGA
AGCAGAGCCGCATCACCCCCATCCAGAAGCCGCGGGGCCTCGACCCTGTGGAGATCCTGCAGGAGCGCGA
GTACAGGCTGCAGGOTCGCATCGCACACCGAATTCAGGAACTTGAAAACCTTCCCGGGTCCCTGGCCGGG
GATTTGCGAACCAAAGCGACCATTGAGCTCAAGGCCCTCAGGCTGCTGAACTTCCAGAGGCAGCTGCGCC
AGGAGGTGGTGGTGTGCATGCGGAGGGACACAGCGCTGGAGACAGCCCTCAATGCTAAGGCCTACAAGCG
CAGCAAGCGCCAGTCCCTGCGCGAGGCCCGCATCACTGAGAAGCTGGAGAAGCAGCAGAAGATCGAGCAG
GAGCGCAAGCGCCGGCAGAAGCACCAGGAATACCTCAATAGCATTCTCCAGCATGCCAAGGATTTCAAGG
AATATCACAGATCCGTCACAGGCAAAATCCAGAAGCTGACCAAGGCAGTGGCCACGTACCATGCCAACAC
GGAGCGGGAGCAGAAGAAAGAGAACGAGCGGATCGAGAAGGAGCGCATGCGGAGGCTCATGGCTGAAGAT
GAGGAGGGGTACCGCAAGCTCATCGACCAGAAGAAGGACAAGCGCCTGGCCTACCTCTTGCAGCAGACAG
ACGAGTACGTGGCTAACCTCACGGAGCTGGTGCCGCAGCACAAGGCTGCCCAGGTCGCCAAGGAGAAAAA
GAAGAAAAAGAAAAAGAAGAAGGCAGAAAATGCAGAAGGACAGACGCCTGCCATTGGGCCGGATGGCGAG
CCTCTAGACGAGACCAGCCAGATGAGCGACCTCCCGGTGAAGGTGATCCACGTGGAGAGTGGGAAGATCC
TCACAGGCACAGATGCCCCCAAAGCCGGGCAGCTGGAGGCCTGGCTCGAGATGAACCCGGGGTATGAAGT
AGOTCCGAGGTCTGATAGTGAAGAAAGTGGCTCAGAAGAAGAGGAAGAGGAGGAGGAGGAAGAGCAGCCG
AGGCAGCACAGCCTCCCACCCTGCCCGTGGAGGAGAAGAACGAAGATTCCAGATCCAGACAGCGATGACG
TCTCTGAGGTGGACGCGCGGCACATCATTGAGAATGCCAAGCAAGATGTCGATGATGAATATGGCGTGTC
CCAGGCCCTTGCACGTGGCCTGCAGTCCTACTATGCCGTGGCCCATGCTGTCACTGAGAGAGTGGACAAG
CAGTCAGCGCTTATGGTCAATGGTGTCCTCAAACAGTACCAGATCAAAGGTTTGGAGTGGCTGGTGTCCC
TGTACAACAACAACCTGAACGGCATCCTGGCCGACGAGATGGGCCTGGGGAAGACCATCCAGACCATCGC
GCTCATCACGTACCTCATGGAGCACAAACGCATCAATGGGCCCTTCCTCATCATCGTGCCTCTCTCAACG
CTGTCCAACTGGGCGTACGAGTTTGACAAGTGGGCCCCCTCCGTGGTGAAGGTGTCTTACAAGGGATCCC
CAGCAGCAAGACGGGCCTTTGTCCCCCAGCTCCGGAGTGGGAAGTTCAACGTCTTGCTGACGACGTACGA
GTACATCATCAAAGACAAGCACATCCTCGCCAAGATCCGTTGGAAGTACATGATTGTGGACGAAGGTCAC
CGCATGAAGAACCACCACTGCAAGCTGACGCAGGTGCTCAACACGCACTATGTGGCACCCCGCCGCCTGC
TGCTGACGGGCACACCGCTGCAGAACAAGCTTCCCGAGCTCTGGGCGCTGCTCAACTTCCTGCTGCCCAC
CATCTTCAAGAGCTGCAGCACCTTCGAGCAGTGGTTTAACGCACCCTTTGCCATGACCGGGGAAAAGGTG
GACCTGAATGAGGAGGAAACCATTCTCATCATCCGGCGTCTCCACAAAGTGCTGCGGCCCTTCTTGCTCC
GACGACTCAAGAAGGAAGTCGAGGCCCAGTTGCCCGAAAAGGTGGAGTACGTCATCAAGTGCGACATGTC
TGCGCTGCAGCGAGTGCTCTACCGCCACATGCAGGCCAAGGGCGTGCTGCTGACTGATGGCTCCGAGAAG
GACAAGAAGGGCAAAGGCGGCACCAAGACCCTGATGAACACCATCATGCAGCTGCGGAAGATCTGCAACC
ACCCCTACATGTTCCAGCACATCGAGGAGTCCTTTTCCGAGCACTTGGGGTTCACTGGCGGCATTGTCCA
AGGGCTGGACCTGTACCGAGCCTCGGGTAAATTTGAGCTTCTTGATAGAATTCTTCCCAAACTCCGAGCA
ACCAACCACAAAGTGCTGCTGTTCTGCCAAATGACCTCCCTCATGACCATCATGGAAGATTACTTTGCGT
ATCGCGGCTTTAAATACCTCAGGCTTGATGGAACCACGAAGGCGGAGGACCGGGGCATGCTGCTGAAAAC
CTTCAACGAGCCCGGCTCTGAGTACTTCATCTTCCTGCTCAGCACCCGGGCTGGGGGGCTCGGCCTGAAC
CTCCAGTCGGCAGACACTGTGATCATTTTTGACAGCGACTGGAATCCTCACCAGGACCTGCAAGCGCAGG
ACCGAGCCCACCGCATCGGGCAGCAGAACGAGGTGCGTGTGCTCCGCCTCTGCACCGTCAACAGCGTGGA
GGAGAAGATCCTAGCTGCAGCCAAGTACAAGCTCAACGTGGACCAGAAGGTGATCCAGGCCGGCATGTTC
GACCAGAAGTCCTCCAGCCATGAGCGGCGCGCCTTCCTGCAGGCCATCCTGGAGCACGAGGAGCAGGATG
AGAGCAGACACTGCAGCACGGGCAGCGGCAGTGCCAGCTTCGCCCACACTGCCCCTCCGCCAGCGGGCGT
CAACCCCGACTTGGAGGAGCCACCTCTAAAGGAGGAAGACGAGGTGCCCGACGACGAGACCGTCAACCAG
ATGATCGCCCGGCACGAGGAGGAGTTTGATCTGTTCATGCGCATGGACCTGGACCGCAGGCGCGAGGAGG
CCCGCAACCCCAAGCGGAAGCCGCGCCTCATGGAGGAGGACGAGCTCCCCTCGTGGATCATCAAGGACGA
CGCGGAGGTGGAGCGGCTGACCTGTGAGGAGGAGGAGGAGAAGATGTTCGGCCGTGGCTCCCGCCACCGC
AAGGAGGTGGACTACAGCGACTCACTGACGGAGAAGCAGTGGCTCAAGGCCATCGAGGAGGGCACGCTGG
AGGAGATCGAAGAGGAGGTCCGGCAGAAGAAATCATCACGGAAGCGCAAGCGAGACAGCGACGCCGGCTC
CTCCACCCCGACCACCAGCACCCGCAGCCGCAGACAAGACGACGAGAGCAAGAAGCAGAAGAAGCGCGGG
CGGCCGCCTGCCGAGAAACTCTCCCCTAACCCACCCAACCTCACCAAGAAGATGAAGAAGATTGTGGATG
CCGTGATCAAGTACAAGGACAGCAGCAGTGGACGTCAGCTCAGCGAGGTCTTCATCCAGCTGCCCTCGCG
AAAGGAGCTGCCCGAGTACTACGAGCTCATCCGCAAGCCCGTGGACTTCAAGAAGATAAAGGAGCGCATT
CGCAACCACAAGTACCGCAGCCTCAACGACCTAGAGAAGGACGTCATGCTCCTGTGCCAGAACGCACAGA
CCTTCAACCTGGAGGGCTCCCTGATCTATGAAGACTCCATCGTCTTGCAGTCGGTCTTCACCAGCGTGCG
GCAGAAAATCGAGAAGGAGGATGACAGTGAAGGCGAGGAGAGTGAGGAGGAGGAAGAGGGCGAGGAGGAA
GGCTCCGAATCCGAATCTCGGTCCGTCAAAGTGAAGATCAAGCTTGGCCGGAAGGAGAAGGCACAGGACC
GGCTGAAGGGCGGCCGGCGGCGGCCGAGCCGAGGGTCCCGAGCCAAGCCGGTCGTGAGTGACGATGACAG
TGAGGAGGAACAAGAGGAGGACCGCTCAGGAAGTGGCAGCGAAGAAGACTGAGCCCCGACATTCCAGTCT
CGACCCCGAGCCCCTCGTTCCAGAGCTGAGATGGCATAGGCCTTAGCAGTAACGGGTAGCAGCAGATGTA
GTTTCAGACTTGGAGTAAAACTGTATAAACAAAAGAATCTTCCATATTTATACAGCAGAGAAGCTGTAGG
ACTGTTTGTGACTGGCCCTGTCCTGGCATCAGTAGCATCTGTAACAGCATTAACTGTCTTAAAGAGA
SMARCA3
SEQ ID NO:133
>gi | 4507070 | ref | NM_003071.1 | Homo sapiens SWI/SNF related, matrix
associated, actin dependent regulator of chromatin, subfamily a,
member 3 (SMARCA3), mRNA
ATTCCCGGGGTCTGACTGGACTCGCGGCGACTTACCTTTCAGTCGTGCGCTCCTGATCCGGCGCTCGGAA
TTTGTCCCCGGCTTCAGGGCTGCGGGGCCTGGAAGGAGGCGTATCGAGGCGGCTCGAAAACGATCCAGGG
GAGCCGAGGCGCTCCTCTTGTCATCCCACTCAGCGCCATGTCCTGGATGTTCAAGAGGGATCCAGTTTGG
AAGTACTTGCAGACTGTCCAGTATGGAGTTCATGGAAATTTTCCACGCCTCTCATATCCAACTTTCTTTC
CACGTTTTGAATTCCAAGATGTTATCCCTCCAGATGACTTTCTAACTAGTGATGAAGAAGTAGATTCCGT
TTTATTTGGAAGTTTGAGAGGTCATGTGGTTGGACTACGCTATTACACGGGAGTAGTTAATAATAATGAA
ATGGTTGCATTACAACGAGATCCTAATAACCCTTATGATAAGAATGCAATTAAAGTAAACAATGTGAATG
GAAATCAAGTTGGCCATTTAAAGAAAGAGCTTGCAGGTGCTTTGGCCTATATCATGGACAACAAATTGGC
ACAAATTGAAGGGGTAGTTCCTTTTGGTGCAAACAATGCTTTTACCATGCCTCTGCATATGACTTTTTGG
GGAAAAGAAGAAAATAGAAAAGCGGTTTCAGATCAGTTGAAGAAACATGGATTTAAATTGGGTCCTGCAC
CAAAAACTTTAGGATTCAATTTGGAAAGTGGTTGGGGCTCTGGAAGAGCTGGACCAAGCTATAGTATGCC
AGTGCATGCTGCAGTACAGATGACAACTGAACAGCTTAAAACAGAATTTGACAAATTGTTTGAAGATTTA
AAAGAAGATGATAAAACCCATGAAATGGAACCAGCTGAGGCTATTGAAACACCACTGCTTCCACATCAAA
AACAAGCTCTAGCTTGGATGGTGTCACGGGAAAATAGCAAAGAACTTCCACCATTCTGGGAACAGCGAAA
TGACTTATACTATAACACAATAACAAATTTTTCTGAGAAGGACCGACCAGAAAATGTCCATGGAGGAATT
TTAGCTGATGATATGGGTTTGGGTAAAACTCTTACAGCCATTGCAGTAATCCTTACCAACTTCCATGATG
GCAGACCTCTTCCTATTGAAAGAGTTAAAAAGAATCTACTGAAGAAGGAATATAATGTTAACGATGACTC
TATGAAACTTGGAGGAAACAATACCAGTGAAAAGGCAGATGGACTAAGCAAAGACGCATCTAGATGTAGT
GAACAACCCAGTATTTCAGATATCAAGGAGAAGAGTAAGTTTCGCATGTCAGAATTGTCTACGTCCCGCC
CCAAAAGAAGAAAAACTGCTGTCCAGTACATAGAAAGCAGTGATTCAGAGGAAATTGAAACAAGTGAATT
GCCGCAGAAAATGAAAGGCAAACTGAAAAATGTACAGTCTGAAACTAAAGGCAGGGCGAAAGCAGGATCT
TCTAAGGTTATAGAAGATGTGGCATTTGCATGTGCATTAACTTCATCTGTTCCTACAACAAAAAAGAAAA
TGTTGAAAAAGGGAGCTTGTGCAGTGGAGGGGTCAAAGAAAACTGATGTTGAGGAGAGACCAAGAACAAC
ACTGATCATCTGTCCGCTTTCTGTGTTAAGCAACTGGATTGACCAGTTTGGACAACATATAAAATCAGAT
GTACACTTGAATTTTTATGTTTATTATGGTCCTGATCGTATTAGAGAACCGGCCTTACTTTCAAAACAGG
ATATTGTTTTGACTACGTATAATATTTTAACTCATGACTATGGAACTAAAGGAGATAGTCCATTACATAG
CATAAGGTGGCTAAGAGTGATCCTGGATGAAGGACATGCCATACGAAATCCAAATGCTCAGCAGACAAAA
GCTGTACTTGACTTAGAATCAGAAAGAAGATGGGTTTTGACAGGTACTCCAATCCAGAATTCTTTAAAGG
ACTTGTGGTCTCTTCTTTCCTTTTTAAAACTTAAACCATTTATTGATAGAGAATGGTGGCATAGAACAAT
ACAGCGTCCTGTCACAATGGGAGATGAAGGAGGACTTAGGCGTTTACAGTCCCTAATTAAAAATATTACA
CTTAGAAGAACAAAGACAAGCAAAATTAAAGGAAAACCTGTTTTGGAGTTACCAGAACGTAAAGTATTTA
TTCAGCACATTACACTTTCAGATGAAGAGAGAAAGATTTATCAGTCTGTGAAAAATGAAGGCAGAGCCAC
TATTGGAAGGTATTTTAATGAAGGGACTGTCCTGGCACATTATGCTGATGTCCTGGGTCTTTTGCTTAGA
CTGCGGCAAATTTGTTGCCATACTTACCTTCTTACAAATGCAGTGTCTTCCAATGGCCCCTCAGGAAATG
ATACACCTGAAGAACTGAGAAAGAAGTTAATAAGGAAGATGAAGTTAATTCTGAGCTCAGGTTCAGATGA
GGAATGTGCAATTTGCCTGGATTCTTTAACAGTTCCTGTGATAACACATTGTGCACATGTATTTTGTAAA
CCCTGTATTTGCCAAGTCATTCAGAATGAGCAGCCACATGCTAAATGCCCTTTATGCAGAAATGATATAC
ATGAAGATAATTTATTTAGAATGTCCTCCAGAAGAATTAGCACGTGACAGTGAGAAAAGTCTGATATGGA
ATGGACATCCAGTTCAAAGATTAATGCGCTAATGCACGCATTGACTGACTTAAGAAAGAAGAATCCCAAC
ATAAAAAGTTTGGTTGTTTCTCAGTTTACAACATTCCTGTCTTTAATAGAAATACCACTTAAAGCCTCTG
GATTTGTGTTTACTCGTTTGGATGGTTCCATGGCCCAAAAGAAAAGAGTTGAATCAATTCAGTGTTTTCA
AAACACTGAAGCAGGATCTCCAACTATAATGCTTCTGTCCTTAAAAGCAGGTGGAGTTGGTTTGAATCTG
TCTGCAGCTTCTCGAGTGTTTTTAATGGATCCAGCCTGGAATCCTGCTGCTGAAGATCAGTGCTTTGACA
GATGCCATAGACTTGGTCAGAAGCAAGAAGTTATCATCACAAAATTCATTGTAAAGGACTCTGTTGAAGA
AAATATGCTGAAAATACAAAACAAAAAGAGAGAACTTGCAGCAGGAGCTTTTGGAACTAAAAAACCAAAT
GOTGACGAAATGAAACAAGCCAAAATTAATGAAATCAGAACATTAATTGACTTATAATTTGTGGGATTTT
AGTAAGAAGACTACTATATGTGAGAGGCGTGATATCTGGATGGAAGTTGGGCTGGATGATCTCCAAAGTC
GTTTCAACTCTTAAAGACATCTTAATCCTGAATGTAAACAATTGTTATGTGTTTAGAATCAGAATTTGAT
TTTGAACTTGAGTAATTCATCCTTACAGCTATCTGTAGAATTAGTCATCTTTTTTCTT
SMARCAL1
SEQ ID NO:134
>gi | 7657149 | ref | NM_014140.1 | Homo sapiens SWI/SNF related, matrix
associated, actin dependent regulator of chromatin, subfamily a-like
1 (SMARCAL1), mRNA
GCTGGGTTGGAAAAAGACTATGTTAGCAAGTGTCACGCCATGCTTTTGCCAACTTTCCATTTAAAGGTTG
ACATTCCTGCATAAACATTTCTCTGTGAAAATGTCCTTGCCTCTTACAGAGGAGCAGAGGAAAAAGATTG
AAGAGAATCGACAAAAGGCTCTGGCCCGCAGAGCTGAGAAGTTATTGGCAGAACAGCATCAGAGGACTAG
CTCGGGCACCTCCATTGCTGGCAACCCATTCCAGGCCAAGCAAGGCCCATCCCAAAATTTCCCAAGGGAG
TCTTGTAAGCCAGTGAGCCATGGTGTCATTTTCAAGCAACAGAATOTCAGTAGCTCATCTAATGCTGACC
AAAGACCTCATGATTCCCACAGTTTTCAGGCAAAGGGAATATGGAAAAAGCCAGAAGAAATGCCCACAGC
CTGCCCAGGCCACAGTCCACGTAGTCAAATGACTCTCACTGGAATCTCTCCTCCCTTGGCACAAAGTCCT
CCAGAGGTCCCTAAACAACAGOTCTTGAGTTATGAGTTAGGTCAAGGTCATGCTCAGGCTTCACCTGAGA
TCAGGTTCACACCCTTTGCTAACCCAACTCATAAGCCTCTGGCCAAACCAAAGAGTTCCCAAGAGACACC
AGCTCATTCCTCTGGACAGCCTCCCAGGGATGCTAAGTTAGAGGCCAAGACAGCAAAAGCCTCCCCTTCG
GGGCAGAACATTTCTTACATCCATTCTAGCTCAGAGAGTGTAACGCCCAGGACAGAAGGAAGACTCCAGC
AGAAGTCAGGGTCCTCAGTCCAAAAAGGAGTGAACTCTCAGAAGGGAAAGTGCGTAAGGAACGGCGATCG
TTTCCAGGTGTTGATTGGGTACAATGCGGAACTCATTGCAGTGTTTAAGACCCTGCCCAGCAAGAATTAT
GATCCTGACACCAAGACGTGGAACITCAGCATGAATGACTATAGTGCCCTGATGAAAGCAGCCCAGAGCC
TCCCCACGGTCAACCTGCAGCCTCTGGAATGGGCCTATGGGAGCAGCGAGTCACCCTCCACCAGCAGTGA
GGGACAGGCCGGCCTTCCATCAGCTCCATCCCTTTCATTTGTCAAAGGGCGAGGCATGCTCATCTCCAGG
GCCTACTTCGAGGCAGACATCAGTTATTCACAGGACCTTATTGCGCTTTTTAAACAGATGGATTCCAGAA
GATATGATGTCAAGACCAGGAAGTGGAGCTTTCTCTTGGAAGAGCACAGTAAACTAATTGCAAAGGTGCG
CTGCCTCCCACAAGTTCAGCTGGACCCTCTGCCCACGACTCTCACCCTGGCGTTTGCTTCTCAGCTCAAG
AAGACATCTCTCAGTCTCACGCCAGATGTCCCAGAGGCAGACCTTTCTGAAGTGGACCCCAAGCTCGTGT
CTAATCTGATGCCCTTTCAGAGAGCTGGAGTCAATTTTGCCATAGCCAAAGGAGGCCGCCTGCTGCTCGC
TGACGACATGGGCCTGGGGAAGACCATCCAAGCCATCTGCATCGCAGCCTTTTACCGGAAGGAGTGGCCG
CTCCTGGTGGTGGTGCCATCCTCCGTGCGCTTCACCTGGGAGCAGGCCTTCCTTCGGTGGCTGCCATCTC
TGAGCCCAGATTGCATCAACGTCGTGGTGACTGGGAAGGACCGCCTGACAGCTGGCCTGATCAACATTGT
CAGCTTTGACCTTCTTAGCAAGTTGGAAAAACAGCTAAAAACCCCTTTTAAAGTTGTCATCATTGATGAA
TCTCACTTCCTCAAAAACAGTAGGACTGCCCGCTGTCGAGCAGCTATGCCGGTCCTAAAGGTCGCCAAGA
GGGTGATCCTGTTGTCGGGCACACCAGCCATGTCCCGGCCCGCAGAGCTCTACACGCAGATCATCGCAGT
CAAGCCAACTTTCTTCCCCCAGTTTCATGCCTTTGGACTTCGCTACTGTGATGCCAAACGGATGCCTTGG
GGGTGGGACTACTCAGGTTCCTCCAACCTGGGAGAGCTGAAGCTCCTGCTGGAGGAAGCAGTCATGCTGC
GGCGCCTCAAGTCCGACGTCCTTTCCCAGCTGCCTGCCAAGCAGCGCAAGATAGTGGTGATTGCCCCAGG
ACGGATCAATGCCAGGACCAGAGCTGCCCTGGATGCTGCAGCCAAGGAAATGACCACCAAGGACAAAACT
AAACAGCAGCAGAAAGATGCCCTCATTCTCTTCTTCAACAGAACAGCTGAAGCTAAAATCCCATCTGTCA
TTGAATATATCTTGGACCTACTGGAAAGTGGAAGAGAGAAGTTTTTAGTATTTGCACACCATAAGGTGGT
CCTGGACGCAATTACGCAAGAGCTTGAGAGAAAGCACGTGCAGCACATCCGCATCGATGGCTCCACCTCA
TCAGCTGAGCGGGAGGACCTGTGCCAGCAGTTCCAACTGTCGGAGAGGCATGCTGTGGCCGTGCTGTCCA
TCACCGCTGCCAATATGGGCCTCACCTTCTCCTCGGCTGACCTGGTGGTGTTTGCTGAGCTGTTTTGGAA
CCCAGGGGTGCTGATCCAGGCTGAGGACCGCGTGCACCGCATTGGACAGACCAGCTCCGTGGGCATTCAC
TACCTCGTGGCAAAGGGCACAGCTGATGACTACCTTTGGCCCCTGATTCAAGAGAAGATTAAAGTTCTGG
CAGAAGCCGGGCTTTCTGAGACCAATTTTTCAGAAATGACAGAATCCACTGATTACCTCTACAAGGACCC
AAAGCAGCAGAAGATGTACGACCTATTCCAGAAGTCCTTTGAGAAAGAAGGAAGTGATATGGACCTCGTG
GAAGCAGCAGAGTCCTTTGACCCAGGAAGTGGTTCAGGAACATCTGGAAGTAGTTCCCAGAACATGGGAG
ACACCCTGGATGAAAGCTCATTGACAGCCAGTCCACAGAAGAAAAGGAGATTTGAATTTTTTGATAACTG
GGACAGCTTTACGTCTCCCCTGTAAAAGGGGCAAAAAAAAAAAAAAAAAAAGCATTTTAAAATCAAAAAG
AAAAAAATAAAA
SMARCA1
SEQ ID NO:135
>gi | 4507066 | ref | NM_003069.1 | Homo sapiens SWI/SNF related, matrix
associated, actin dependent regulator of chromatin, subfamily a,
member 1 (SMARCA1), mRNA
CAAGACTGGAAGCAGAGAGAGAGAGCAAGAGTGAGAGAGAGCGAGCGAGCGTAGTCAGGAGATGGTGTTG
TATTCCAGGAGAAAACGTTTCTTCATTTCAACTCAAACTTGCTGCTAAAGCGCCCTAAATCTGAAAAGGA
AATGGACCCAGAATATGAAGAGAAAATGAAAGCCGACCGAGCAAAGAGATTTGAATTTTTACTGAAGCAG
ACAGAACTTTTTACATTTCATTCAGCCTTCAGCACAGAAATCTCCAAAACATCTCCACTGAACATGAAAT
TGGGACGTCCCCGAATAAAGAAAGATGAAAAGCAGAGCTTAATTTCTGCTGGAGACTACCGCCATAGGCG
CACAGAGCAAGAAGAAGATGAAGAGCTACTGTCTGAGAGTCGGAATACATCTAATGTGTGTATTAGATTT
GAGGTGTCACCTTCATATGTGAAAGGGGGGCCACTGAGAGATTATCAGATTCGAGGACTGAATTGGTTGA
TCTCTTTATATGAAAATGGAGTCAATGGCATTTTGGCTGATGAAATGGGCCTTGGGAAAACTTTACACAC
AATTGCTTTGCTTGGTTACCTGAAACACTACCGAAATATTCCTGGACCTCACATGGTTTTAGTTCCAAAG
TCTACTTTACACAACTGGATGAATGAATTTAAACGATGGGTCCCATCTCTCCGTGTCATTTGTTTTGTCG
GAGACAAGGATGCCAGAGCTGCTTTTATTCGTGATGAAATGATGCCAGGAGAGTGGGATGTTTGCGTTAC
TTCTTATGAGATGGTAATTAAAGAAAAATCTGTATTCAAAAAGTTTCACTGGCGATACCTGGTCATTGAT
GAAGCTCACAGAATAAAGAATGAAAAATCTAAGCTTTCAGAGATTGTTCGTGAGTTCAAGTCGACTAACC
GCTTGCTCCTAACTGGAACACCTTTGCAGAATAACCTGCATGAACTGTGGGCCTTACTCAACTTTTTATT
GCCTGATGTCTTTAATTCTGCAGATGACTTTGATTCTTGGTTTGACACTAAAAATTGTCTTGGTGATCAA
AAACTCGTGGAAAGACTTCATGCAGTTTTAAAACCATTTTTGTTACGCCGTATAAAAACTGATGTAGAGA
AGAGTCTGCCACCTAAAAAGGAAATAAAGATTTACTTGGGGCTGAGTAAGATGCAACGAGAATGGTATAC
AAAAATCCTGATGAAAGATATTGATGTTTTAAACTCTTCTGGCAAGATGGACAAGATGCGACTCTTAAAC
ATTCTGATGCAGCTTCGAAAGTGTTGTAATCATCCATATCTGTTTGATGGTGCTGAACCTGGTCCACCTT
ATACCACTGATGAGCATATTGTCAGCAACAGTGGTAAAATGGTAGTTCTGGATAAACTATTGGCCAAACT
CAAAGAACAGGGTTCAAGGGTTCTCATTTTCAGCCAGATGACTCGCTTGCTGGATATTTTGGAAGATTAT
TGCATGTGGCGTGGTTATGAGTATTGTCGACTGGATGGACAAACCCCGCATGAAGAAAGAGAGGATAAAT
TCCTAGAAGTGGATTTCTGGGTCAAAGGGAAGCAATAGAGGCTTTTAATGCTTCCTAATAGTAGCAAATT
CATCTTTATGCTAAGTACCAGGGCTGGAGGTCTCGGATTTAACCTGGCAAGTGCTGATGTGGTTATACTA
TATGATTCAGACTGGAACCCACAGGTTGATCTACAAGCTATGGATCGAGCACATCGTATTGGTCAGAAGA
AACCAGTACGTGTATTCCGTCTCATCACTGACAACACTGTTGARGAGAGGATTGTAGAAAGAGCTGAGAT
AAAACTGAGACTCGATTCAATTGTTATACAACAAGGAAGACTCATTGACCAACGGTCTAACAAGCTGGCA
AAAGAGGAAATGTTACAAATGATACGGCATGGAGCCACCCATGTTTTTGCTTCTAAAGAGAGTGAGTTGA
CAGATGAAGACATTACAACTATTCTGGAAGAGGGGAAAAAGAAGACTGCAGAGATGAATGAACGCCTGCA
AAAAATGGGAGAGTCTTCTCTAAGAAATTTTAGAATGGACATTGAACAAAGTTTATACAAATTTGAGGGA
GAAGATTATAGAGAAAACAGAAGCTTGGCATGGTGGAATGGATTGAACCTCCTAAACGAGAAACGCAAAG
CAAACTACGCAGTGGATGCCTACTTTAGAGAGGCTTTGCGTGTCAGCGAGCCAAAGATTCCAAAGGCTCC
ACGGCCTCCAAAACAGCCAAATGTTCAGGATTTTCAATTTTTCCCACCACGCTTATTTGAGCTCCTGGAA
AAGGAAATTCTTTATTATCGGAAGACAATAGGCTATAAGGTTCCAAGGAATCCTGATATCCCAAATCCAG
CTCTGGCTCAAAGAGAAGAGCAAAAAAAGATTGATGGAGCTGAACCTCTTACACCAGAAGAGACTGAAGA
AAAGGAAAAACTTCTCACACAAGGTTTCACAAACTGGACTAAACGAGATTTTAACCAGTTTATTAAAGCT
AATGAGAAATATGGAAGAGATGACATTGATAACATAGCTCGAGAGGTAGAGGGCAAATCCCCTGAGGAGG
TCATGGAGTATTCAGCTGTATTTTGGGARCGTTGCAATGAATTACAGGACATTGAGAAAATTATGGCTCA
AATTGAACGTGGAGAAGCAAGAATTCAACGAAGGATCAGTATCAAGAAAGCCCTGGATGCCAAAATTGCA
AGATACAGGCTCCATTTCATCAGTTGCGCATTTCAGTATGGAACCAGCAAAGGAAAGAACTATACTGAGG
AAGAAGATAGATTCTTGATTTGTATGTTACACAAAATGGGCTTTGATAGAGAAAATGTATATGAAGAATT
AAGACAGTGTGTACGAAATGCTCCCCAGTTTAGATTTGACTGGTTTATCAAGTCTAGGACTGCCATGGAA
TTCCAGAGACGCTGTAACACTCTGATTTCATTGATTGAGAAAGAAAATATGGAAAATGAGGAAAGAGAGA
GAGCAGAAAAGAAGAAACGGGCAACTAAAACTCCAATGGTAAAATTTTCAGCATTTTCCTAACTTTTAGA
TTTAACATTGTTGGGCCATTTAAAATGTGCATATTGGAGCAGAACATTAAATCTGTTTCCATTTTAGTCA
CAGAAAAGAAAAGCAGAGTCAGCTACTGAGAGCTCTGGAAAGAAGGATGTCAAGAAGGTGAAATCCTAAA
GCCTAGAAATAAAGTTTTAAATGGGAAACTGCTATTTTCTTGTTCCCATCTTCAAATGCTAATTGCCAGT
TCCAGTGTATTCATGGTACTCTAAGAAAAATCTCTTTGGTTTTGATTTCTTGCATATTTTATATATTTTA
CAATGCTTTCTACCTGAAATGTGTAGCTTTATATTTTATGGCATTCTAGTATTTTrGTGTACTGTAITTT
GTGCATTTCATGTCTTCATCAAATTTCCTCTCAGTCCTTGTTCTTTTGAAGCTTGTGCTGAGGTTTAGCT
TTTCTATGTTTTATATGCCGCTGCTTTGAAAGAGAACCTAGATTCTATAGTTGTATTATTGTTGTTTCAT
ACTTTAAATTTATATGGCTGTGGAAAAACGAATTAAAATGTTTTGAGGAGAAAGAAAAAAA
CHRAC1
SEQ ID NO:136
>gi | 8393115 | ref | NM_017444.1 | Homo sapiens chromatin accessibility
complex 1 (CHRAC1), mRNA
ATGGCGGACGTGGTCGTGGGTAAAGACAAGGGCGGGGAGCAGCGGTGCATCTCGCTGCCTCTATCCCGCA
TCCGGGTCATCATGAAGAGCTCCCCCGAGGTGTCCAGCATCAACCAGGAGGCGTTGGTGCTCACGGCCAA
GGCCACGGAGCTCTTTGTTCAATGCCTAGCCACCTATTCCTACAGACACGGCAGTGGAAAGGAAAAGAAA
GTACTGACTTACAGTGATTTAGCAAACACTGCACAGCAATCAGAAACTTTTCAGTTTCTTGCAGATATAT
TACCAAAGAAGATTTTAGCTAGTAAATACCTGAAAATGCTTAAAGAGGAAAAGAGGGAAGAAGATGAGGA
GAATGACAATGATAATGAAAGTGACCATGATGAAGCTGACTCCTAA

1-21. (canceled)

22. An inhibitory RNA (RNAi) molecule derived from a nucleic acid molecule comprising a nucleic acid sequence selected from the group consisting of:
a) nucleic acid sequences represented by any of SEQ ID NOs:7-23 or fragments thereof;
b) nucleic acid sequences which hybridize to any of the nucleic acid sequences of SEQ ID NOs:7-23 and which encode a Notch signaling target gene; and
c) nucleic acid sequences which comprise sequences which are degenerate as a result of the genetic code to the nucleic acid sequences defined in a) and b).

23. An RNAi molecule according to claim 22, wherein said molecule comprises a first part linked to a second part, wherein said first and second parts are complementary over at least part of their length, and further wherein said first and second parts form a double stranded region by complementary base pairing over at least part of their length.

24. An RNAi molecule according to claim 23, wherein said first and second parts are linked by at least one nucleotide base.

25. An RNAi molecule according to claim 24, wherein said first and second parts are linked by 2, 3, 4, 5, 6, 7, 8, 9 or 10 nucleotide bases.

26. An RNAi molecule according to claim 24, wherein the linker is at least 10 nucleotide bases.

27. An RNAi molecule according to claim 22, wherein the length of said RNAi molecule is between 10 and 1000 nucleotide bases (nb).

28. An RNAi molecule according to claim 27, wherein the length of the RNA molecule is selected from 10 nb, 20 nb, 30 nb, 40 nb, 50 nb, 60 nb, 70 nb, 80 nb and 90 nb.

29. An RNAi molecule according to claim 27, wherein the RNA is 21 nb in length.

30. An RNAi molecule according to claim 27, wherein the RNA molecule is 100 nb, 200 nb, 300 nb, 400 nb, 500 nb, 600 nb, 700 nb, 800 nb, 900 nb or 1000 nb in length.

31. An RNAi molecule according to claim 22, wherein the RNA molecule is at least 1000 nb.

32. An RNAi molecule according to claim 22, wherein the RNAi molecule comprises modified nucleotide bases.

33. A nucleic acid molecule encoding at least part of a gene which modulates stem cell differentiation comprising a nucleic acid sequence selected from the group consisting of:
a) nucleic acid sequences represented by SEQ ID NOs:7-23 or fragments thereof;
b) nucleic acid sequences which hybridize to any of the nucleic acid sequences of SEQ ID NOs:7-23 and which are Notch signaling target genes; and
c) nucleic acid sequences which comprise sequences which are degenerate as a result of the genetic code to any of the nucleic acid sequences defined in (a) and (b);
wherein said nucleic acid molecule comprises a first part linked to a second part, said first and second parts being complementary over at least part of their length, said nucleic acid molecule being operably linked to at least one further nucleic acid molecule capable of promoting transcription of said nucleic acid linked thereto, and said first and second parts forming a double stranded region by complementary base pairing over at least part of their length when said nucleic acid molecule is transcribed.

34. An expression vector including an expression cassette comprising at least one nucleic acid molecule according to claim 33.

35. A method of treating an animal in need thereof comprising administering to said animal a therapeutically effective amount of at least one RNAi molecule according to claim 22.

36. A method of treatment according to claim 35, wherein said animal is a human.

37. A method of treating an animal in need thereof comprising administering to said animal an therapeutically effective amount of a vector according to claim 34.

38. A method of treatment according to claim 37, wherein said animal is a human.

39. An in vitro method to modulate the differentiation state of a pluripotential stem cell comprising the steps of:
i) contacting a pluripotential stem cell with at least one inhibitory RNA molecule (RNAi) comprising a sequence of a gene which mediates at least one step in the differentiation of said cell, wherein said gene is selected from the group consisting of:
a) nucleic acid sequences represented by any of SEQ ID NOs:7-23 or fragments thereof;
b) nucleic acid sequences which hybridize to any of the nucleic acid sequences of SEQ ID NOs:7-23 and which are Notch signaling target genes; and
c) nucleic acid sequences which comprise sequences which are degenerate as a result of the genetic code to the nucleic acid sequences defined in a) and b);
(ii) providing conditions conducive to the proliferation of the cell treated in (i) above; and
(iii) optionally maintaining or storing said cell.

40. A lineage restricted stem cell or a differentiated stem cell obtainable by the method according to claim 39.

41. A lineage restricted stem cell according to claim 40, wherein said cell is selected from the group consisting of hemopoietic stem cells, neural stem cells, bone stem cells, muscle stem cells, mesenchymal stem cells, trophoblastic stem cells, epithelial stem cells and endodennal stem cells.

42. A lineage restricted stem cell according to claim 41, wherein said cell is an epithelial stem cell derived from an organ selected from the group consisting of skin, gastrointestinal mucosa, kidney, bladder, mammary glands, uterus, prostate and endocrine glands, or an endodermal stem cell derived from an organ selected from the group consisting of liver, pancreas, lung and blood vessels.

43. A differentiated cell according to claim 40, wherein said cell is selected from the group consisting of nerve cells, mesenchymal cells, muscle cells, liver cells, kidney cells, blood cells, panceatic cells, epithelial cells and endothelial cells.

44. A differentiated cell according to claim 43, wherein said cell is a cardiomyocyte muscle cell; a blood cell selected from the group consisting of erythrocytes, CD4+ lymphocytes and CD8+ lymphocytes; or an epithelial cell selected from the group consisting of lung cells and gastric cells.

45. A cell culture comprising at least one lineage restricted stem cell or differentiated cell according to any of claim 40.

46. An organ comprising a lineage restricted stem cell or a differentiated stem cell according to claim 40.

47. A method of treating an animal in need thereof comprising administering to said animal a therapeutically effective amount of a cell according to claim 40.

48. A method of treatment according to claim 47, wherein said animal is a human.
